(19) United States
(12) Patent Application Publication
(10) Pub. No.: US 2025/0066781 A1
(43) Pub. Date: Feb. 27, 2025

(54) METHODS FOR TREATING CONDITIONS AND DISEASES

(71) Applicant: Stoke Therapeutics, Inc., Bedford, MA (US)

(72) Inventors: Baruch Ticho, Newton, MA (US); Kimberly Parkerson, Phoenix, AZ (US); Meena Meena, Belmont, MA (US); Susovan Mohapatra, Acton, MA (US)

(21) Appl. No.: 18/782,724

(22) Filed: Jul. 24, 2024

Related U.S. Application Data
(60) Provisional application No. 63/515,284, filed on Jul. 24, 2023.

(51) Int. Cl. C12N 15/113; A61P 25/08

(52) U.S. Cl. CPC C12N 15/113; A61P 25/08; C12N 2310/11

(57) ABSTRACT

Provided herein are methods for treating conditions and diseases characterized by SCN1A, SCN8A or SCN5A protein deficiency by targeting the alternative splicing events in SCN1A gene and modulating the expression level of functional proteins in Dravet Syndrome patients and/or inhibit aberrant protein expression.

### CROSS REFERENCE

This application claims benefit of U.S. Provisional Patent Application No. 63/515,284 filed Jul. 24, 2023, which is incorporated herein by reference in its entirety.

### SEQUENCE LISTING

The instant application contains a Sequence Listing which has been submitted electronically in XML format and is hereby incorporated by reference in its entirety. Said XML copy, created on Sep. 24, 2024, is named 47991-747_201_SL.xml and is 1,678,951 bytes in size.

### BACKGROUND

Nervous system disorders are often associated with channelopathy, characterized by the disturbed function of ion channels that mediate neuronal excitability, neuronal interactions, and brain functions at large. Mutations in the SCN1A gene, which is part of the SCN1A-SCN2A-SCN3A gene cluster that encodes alpha-pore forming subunits of the neuronal voltage-gated sodium channel, can result in expression of Na_{V}1.1 protein (also termed as “Na_{V}1.1”) with reduced functions as compared to a wild-type Na_{V}1.1 protein, reduced expression of Na_{V}1.1, or both. Mutations in SCN1A gene are associated with development of disease number of diseases and conditions, such as Dravet Syndrome (DS) (Miller, et al., 1993-2015, GeneReviews, Eds. Pagon R A, et al., Seattle (WA): University of Washington, Seattle, Bookshelf ID: NBK1318, and Mulley, et al., 2005, Hum. Mutat. 25:535-542).

### SUMMARY OF THE DISCLOSURE

Provided herein, in some aspects, is a compound according to the following chemical structure (SEQ ID NO: 1100):

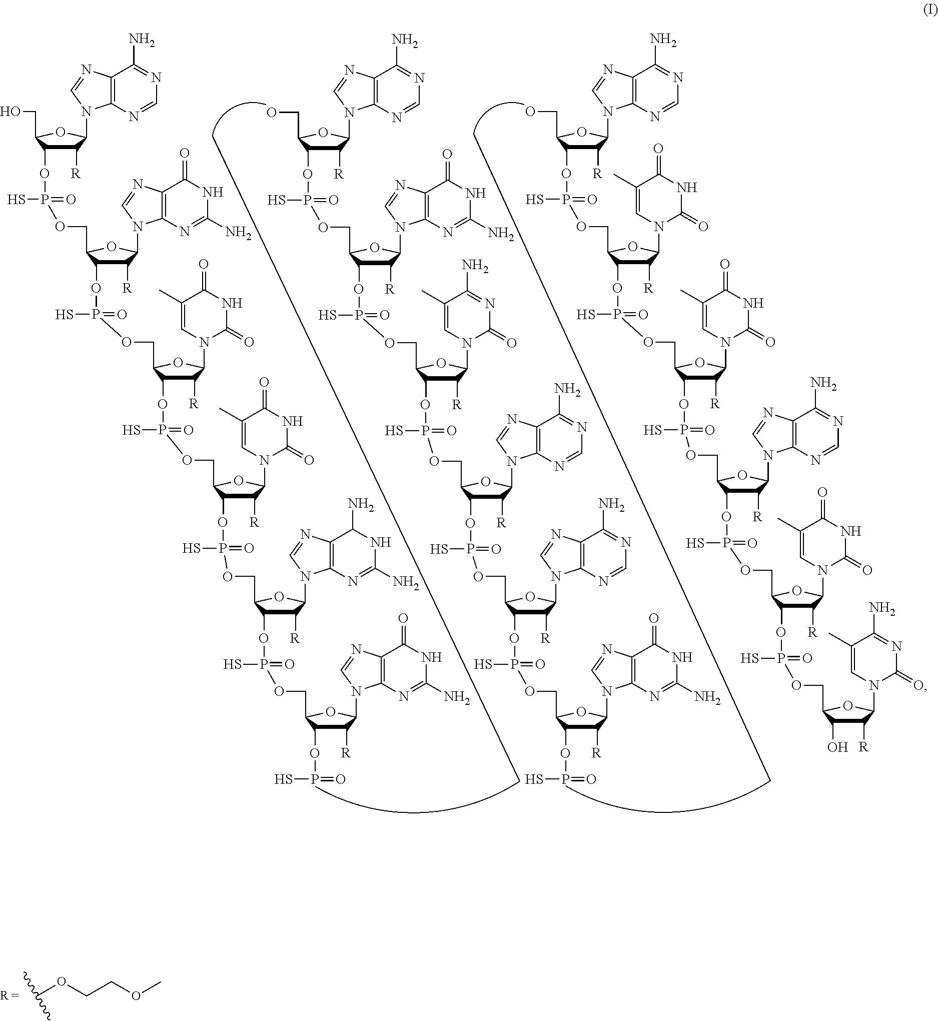

or a salt thereof.

In some cases, the compound has the following chemical structure (SEQ ID NO: 1100):

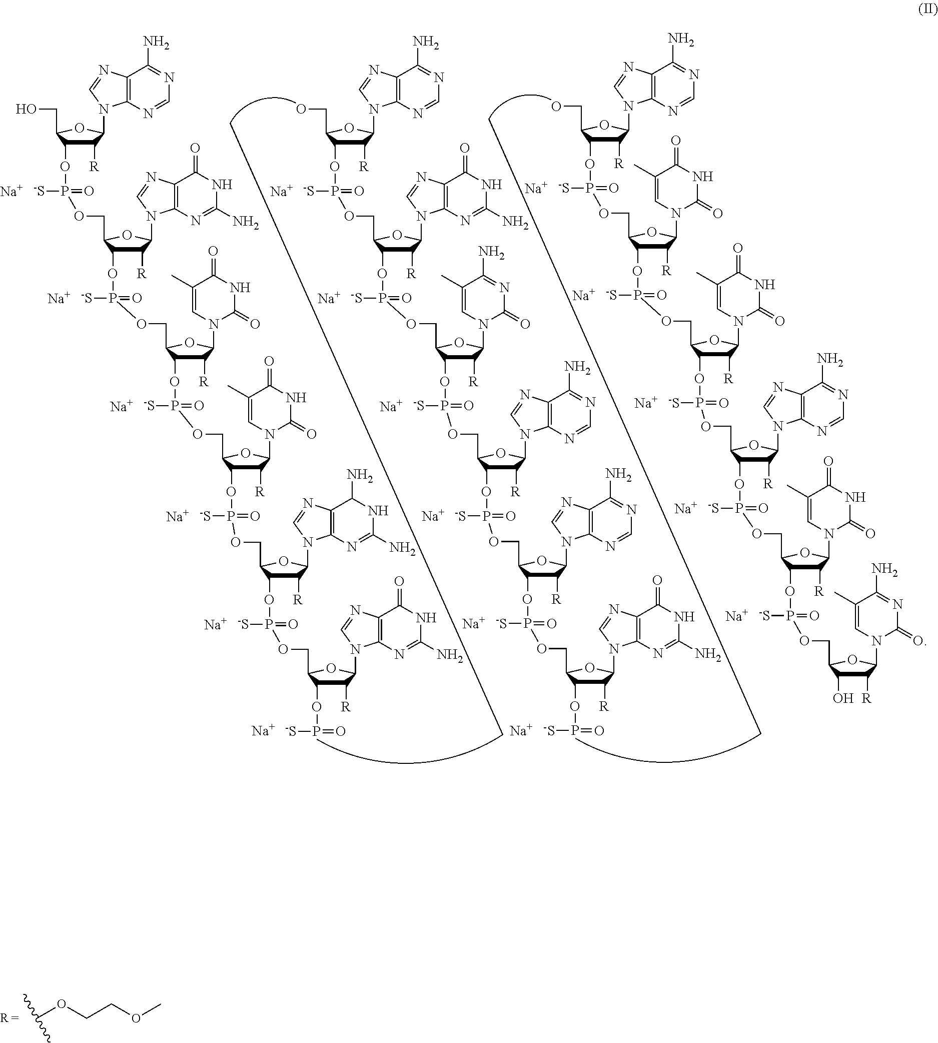

Provided herein, in some aspects, is a method of treating or reducing the likelihood of developing a disease or condition characterized by a reduced expression or function of Na_{V}1.1 protein in a human subject in need thereof, the method comprising administering to the human subject a pharmaceutical composition comprising a compound according to chemical structure (I) as aforementioned, or a salt thereof, thereby treating or reducing the likelihood of developing the disease or condition in the human subject. In some cases, the compound has chemical structure (II) as aforementioned.

Provided herein, in some aspects, is a pharmaceutical formulation comprising: (a) a compound according to chemical structure (I) as aforementioned, or a salt thereof; and (b) a pharmaceutically acceptable diluent, and wherein the compound is dissolved or suspended in a solution. In some cases, the compound has chemical structure (II) as aforementioned.

Provided herein, in some aspects, is a kit comprising: (i) a concentrate comprising a compound according to chemical structure (I) as aforementioned, or a salt thereof; and (ii) a diluent, wherein the concentrate is miscible with the diluent; and (iii) instructions for diluting or solubilizing the compound in the diluent. In some cases, the compound has chemical structure (II) as aforementioned.

Provided herein, in some aspects, is a use of a compound for the manufacture of a medicament for treating or preventing a disease or condition characterized by a reduced expression or function of Na_{V}1.1 protein in a human subject in need thereof, and wherein the compound is according to chemical structure (I) as aforementioned, or a salt thereof. In some cases, the compound has chemical structure (II) as aforementioned.

Provided herein, in some aspects, is a method of treating or reducing the likelihood of developing a disease or condition in a human subject in need thereof, the method comprising administering to the human subject the pharmaceutical composition comprising the compound disclosed herein at a first dose amount of from about 30 to about 70 mg and one or more maintenance doses, wherein the one or more maintenance doses contain a dose amount that is different from the first dose amount. In some cases, the compound is according to the chemical structure (I) as aforementioned, or a salt thereof. In some cases, the compound is according to the chemical structure (II) as aforementioned.

In some embodiments, the method comprises administering to the human subject the pharmaceutical composition comprising the compound at the first dose of about 20, 22.5, 25, 27.5, 30, 32.5, 35, 37.5, 40, 42.5, 45, 47.5, 50, 52.5, 55, 57.5, 60, 62.5, 65, 67.5, 70, 72.5, 75, 77.5, or 80 mg. In some embodiments, the first dose contains the compound at a dose amount of about 30 mg. In some embodiments, the first dose contains the compound at a dose amount of about 70 mg. In some embodiments, the first dose contains the compound at a dose amount of about 45 mg. In some embodiments, the first dose contains the compound at a dose amount of about 50 mg. In some embodiments, the first dose contains the compound at a dose amount of from 25 mg to about 35 mg. In some embodiments, the first dose contains the compound at a dose amount of from 35 to 50 mg. In some embodiments, the first dose contains the compound at a dose amount of from 45 to 70 mg. In some embodiments, the first dose contains the compound at a dose amount of from 50 to 70 mg. In some embodiments, the first dose contains the compound at a dose amount of from 60 to 80 mg. In some embodiments, the first dose contains the compound at a dose amount of from 30 to 45 mg. In some embodiments, the first dose contains the compound at a dose amount of from 25 to 40 mg. In some embodiments, the first dose contains the compound at a dose amount of from 35 to 45 mg. In some embodiments, the first dose contains the compound at a dose amount of from 40 to 55 mg.

In some embodiments, the one or more maintenance doses contain a dose of from about 0.5 milligrams to about 500 milligrams. In some embodiments, the one or more maintenance doses is about 0.1, 0.5, 1, 2.5, 5, 6, 7, 8, 9, 10, 11, 12, 13, 14, 15, 16, 17, 18, 19, 20, 22.5, 25, 27.5, 30, 32.5, 35, 37.5, 40, 42.5, 45, 47.5, 50, 52.5, 55, 57.5, 60, 62.5, 65, 67.5, 70, 72.5, 75, 77.5, 80, 82.5, 85, 87.5, 90, 92.5, 95, 97.5, 100, 102.5, 105, 107.5, 110, 112.5, 115, 117.5, 120, 122.5, 125, 127.5, 130, 132.5, 135, 137.5, 140, 142.5, 145, 147.5, 150, 152.5, 155, 157.5, 160, 162.5, 165, 167.5, 170, 172.5, 175, 177.5, 180, 182.5, 185, 187.5, 190, 192.5, 195, 197.5, 200, 202.5, 205, 207.5, 210, 212.5, 215, 217.5, 220, 222.5, 225, 227.5, 230, 232.5, 235, 237.5, 240, 242.5, 245, 247.5, or 250 mg. In some embodiments, the one or more maintenance doses is about 3 mg, 3.5 mg, 4 mg, 4.5 mg, or 5 mg. In some embodiments, the one or more maintenance doses contain the compound at a dose amount of about 10, 15, 20, 25, 30, 35, 40, 45, 50, 60, 70, or 80 mg. In some embodiments, the one or more maintenance doses contain the compound at a dose amount of about 30 mg. In some embodiments, the one or more maintenance doses contain the compound at a dose amount of about 45 mg. In some embodiments, the one or more maintenance doses contain the compound at a dose amount of about 70 mg. In some embodiments, the one or more maintenance doses contain the compound at a dose amount of from 10 to 45 mg. In some embodiments, the one or more maintenance doses contain the compound at a dose amount of from 15 to 45 mg. In some embodiments, the one or more maintenance doses contain the compound at a dose amount of from 20 to 45 mg. In some embodiments, the one or more maintenance doses contain the compound at a dose amount of from 25 to 45 mg. In some embodiments, the one or more maintenance doses contain the compound at a dose amount of from 30 to 45 mg. In some embodiments, the one or more maintenance doses contain the compound at a dose amount of from 35 to 45 mg. In some embodiments, the one or more maintenance doses contain the compound at a dose amount of from 10 to 40 mg. In some embodiments, the one or more maintenance doses contain the compound at a dose amount of from 15 to 40 mg. In some embodiments, the one or more maintenance doses contain the compound at a dose amount of from 20 to 40 mg. In some embodiments, the one or more maintenance doses contain the compound at a dose amount of from 25 to 40 mg. In some embodiments, the one or more maintenance doses contain the compound at a dose amount of from 30 to 40 mg. In some embodiments, the one or more maintenance doses contain the compound at a dose amount of from 10 to 35 mg. In some embodiments, the one or more maintenance doses contain the compound at a dose amount of from 15 to 35 mg. In some embodiments, the one or more maintenance doses contain the compound at a dose amount of from 20 to 35 mg. In some embodiments, the one or more maintenance doses contain the compound at a dose amount of from 25 to 35 mg. In some embodiments, the one or more maintenance doses contain the compound at a dose amount of from 30 to 35 mg. In some embodiments, the one or more maintenance doses contain the compound at a dose amount of from 10 to 30 mg. In some embodiments, the one or more maintenance doses contain the compound at a dose amount of from 15 to 30 mg. In some embodiments, the one or more maintenance doses contain the compound at a dose amount of from 20 to 30 mg. In some embodiments, the one or more maintenance doses contain the compound at a dose amount of from 25 to 30 mg. In some embodiments, the one or more maintenance doses contain the compound at a dose amount of from 10 to 25 mg. In some embodiments, the one or more maintenance doses contain the compound at a dose amount of from 15 to 25 mg. In some embodiments, the one or more maintenance doses contain the compound at a dose amount of from 20 to 25 mg. In some embodiments, the one or more maintenance doses contain the compound at a dose amount of from 10 to 20 mg. In some embodiments, the one or more maintenance doses contain the compound at a dose amount of from 15 to 20 mg. In some embodiments, the one or more maintenance doses contain the compound at a dose amount of from 55 to 70 mg. In some embodiments, the one or more maintenance doses contain the compound at a dose amount of from 55 to 75 mg. In some embodiments, the one or more maintenance doses contain the compound at a dose amount of from 55 to 80 mg. In some embodiments, the one or more maintenance doses contain the compound at a dose amount of from 40 to 70 mg. In some embodiments, the one or more maintenance doses contain the compound at a dose amount of from 40 to 75 mg. In some embodiments, the one or more maintenance doses contain the compound at a dose amount of from 40 to 80 mg. In some embodiments, the one or more maintenance doses contain the compound at a dose amount of from 65 to 90 mg. In some embodiments, the one or more maintenance doses contain the compound at a dose amount of from 65 to 95 mg. In some embodiments, the one or more maintenance doses contain the compound at a dose amount of from 65 to 100 mg.

In some embodiments, the one or more maintenance doses is higher than the first dose. In some embodiments, the one or more maintenance doses contain the compound at a dose amount that is at least about 10%, 20%, 30%, 40%, 50%, 60%, 70%, or 80% higher than the first dose amount. In some embodiments, the one or more maintenance doses contain the compound at a dose amount that is at least about 5 mg, 10 mg, 15 mg, 20 mg, 30 mg, 50 mg, or 70 mg higher than the first dose amount. In some embodiments, the one or more maintenance doses is higher than the first dose following an indication that administration of the first dose is tolerated. In some embodiments, the one or more maintenance doses is higher than the first dose following an indication that administration of the first dose is not effective. In some embodiments, the one or more maintenance doses is lower than the first dose. In some embodiments, the one or more maintenance doses contain the compound at a dose amount that is at least about 10%, 20%, 30%, 40%, 50%, 60%, 70%, or 80% lower than the first dose amount. In some embodiments, the one or more maintenance doses contain the compound at a dose amount that is at least about 5 mg, 10 mg, 15 mg, 20 mg, 30 mg, 35 mg, 40 mg, or 45 mg lower than the first dose amount.

In some embodiments, the one or more maintenance doses is higher than the first dose following an indication that administration of the first dose is not tolerated. In some embodiments, the one or more maintenance doses is lower than the first dose following an indication that administration of the first dose is not effective. In some embodiments, a subsequent dose of the one or more maintenance doses is higher than a previously administered maintenance dose. In some embodiments, a subsequent dose of the one or more maintenance doses is the same as a previously administered maintenance dose. In some embodiments, a subsequent dose of the one or more maintenance doses is lower than a previously administered maintenance dose.

In some embodiments, the first of the one or more maintenance doses are administered at least 1, 2, 3, 4, 5, 6, 7, 8, 9, 10, 11, or 12 weeks after administration of the first dose. In some embodiments, the first of the one or more maintenance doses is administered at least 1, 2, 3, 4, 5, 6, 7, 8, 9, 10, 11, or 12 months after administration of the first dose. In some embodiments, the one or more maintenance doses is administered 4 weeks after the administration of the first dose. In some embodiments, the one or more maintenance doses is administered 8 weeks after the administration of the first dose. In some embodiments, the first of the one or more maintenance doses are administered at least 1, 2, 3, 4, 5, 6, 7, or 8 weeks after administration of a previous maintenance dose. In some embodiments, a subsequent dose of the one or more maintenance doses is administered at least 1, 2, 3, 4, 5, 6, 7, 8, 9, 10, 11, or 12 months after administration of a previous maintenance dose. In some embodiments, a subsequent dose of the one or more maintenance doses is administered 4 weeks after the administration of a previously administered maintenance dose. In some embodiments, the dose frequency is maintained or reduced following an indication that the previous dose is effective or not tolerated. In some embodiments, the dose frequency is increased following an indication that the previous dose is not effective or tolerated. In some embodiments, the dose frequency is maintained or reduced following an indication that the previous dose is effective. In some embodiments, the dose frequency is increased following an indication that the previous dose is not effective.

In some embodiments, the first dose contains the compound at a dose amount effective to reduce seizure frequency and/or improve cognition and/or behavior for over one year, compared to a subject not treated with the compound. In some embodiments, the one or more maintenance doses contains the compound at a dose amount effective to reduce seizure frequency and/or improve cognition and/or behavior for over one year, compared to observed natural history of the disease. In some embodiments, the first dose contains the compound at a dose amount effective to reduce seizure frequency and/or improve cognition and/or behavior for over six months, compared to a subject not treated with the compound. In some embodiments, the one or more maintenance doses contains the compound at a dose amount effective to reduce seizure frequency and/or improve cognition and/or behavior for over six months, compared to observed natural history of the disease. In some embodiments, the first dose contains the compound at a dose amount effective to reduce seizure frequency and/or improve cognition and/or behavior for over three months, compared to a subject not treated with the compound. In some embodiments, the one or more maintenance doses contain the compound at a dose amount effective to reduce seizure frequency and/or improve cognition and/or behavior for over three months, compared to observed natural history of the disease.

In some embodiments, the pharmaceutical composition comprises a pharmaceutically acceptable excipient, carrier, or diluent. In some embodiments, the pharmaceutical composition is a liquid composition. In some embodiments, the pharmaceutical composition comprises from 0.1 mL to 50 mL of a diluent, wherein the compound is solubilized or diluted in the diluent. In some embodiments, the pharmaceutical composition comprises about 0.1, 0.5, 1, 2, 2.5, 3, 4, 5, 6, 7, 8, 9, 10, 11, 12, 13, 14, 15, 16, 17, 18, 19, 20, 25, 30, 35, 40, 45 or 50 mL of the diluent. In some embodiments, the pharmaceutical composition comprises 1 mL to 20 mL of the diluent, 2 mL to 10 mL of the diluent or 1 mL to 5 mL of the diluent. In some embodiments, the pharmaceutical composition comprises at least 5, 6, 7, 8, 9, or 10 mL of the diluent. In some embodiments, the pharmaceutical composition comprises about 5, 6, 7, 8, 9, or 10 mL of the diluent. In some embodiments, the pharmaceutical composition comprises about 5 mL of the diluent. In some embodiments, the pharmaceutical composition comprises about 7 mL of the diluent. In some embodiments, the pharmaceutical composition comprises about 9 mL of the diluent. In some embodiments, the pharmaceutical composition comprises about 10 mL of the diluent. In some embodiments, the compound is dissolved or suspended in the diluent and the first dose has a volume of 10 mL or higher. In some embodiments, the compound is dissolved or suspended in the diluent and the first dose has a volume of 5 ml or higher. In some embodiments, the compound is dissolved or suspended in the diluent and the first dose has a volume of 10 mL. In some embodiments, the compound is dissolved or suspended in the diluent and the first dose has a volume of 15 mL. In some embodiments, the compound is dissolved or suspended in the diluent and the first dose has a volume of 20 mL. In some embodiments, the compound is dissolved or suspended in the diluent and the first dose has a volume of 25 mL. In some embodiments, the compound is dissolved or suspended in the diluent and the first dose has a volume of 30 mL. In some embodiments, the compound is dissolved or suspended in the diluent and each of the one or more maintenance doses has a volume of 5 mL or higher. In some embodiments, the compound is dissolved or suspended in the diluent and each of the one or more maintenance doses has a volume of 10 mL or higher. In some embodiments, the compound is dissolved or suspended in the diluent and each of the one or more maintenance doses has a volume of 5 mL. In some embodiments, the compound is dissolved or suspended in the diluent and each of the one or more maintenance doses has a volume of 10 mL. In some embodiments, the compound is dissolved or suspended in the diluent and each of the one or more maintenance doses has a volume of 15 mL. In some embodiments, the compound is dissolved or suspended in the diluent and each of the one or more maintenance doses has a volume of 20 mL. In some embodiments, the compound is dissolved or suspended in the diluent and each of the one or more maintenance doses has a volume of 25 mL. In some embodiments, the compound is dissolved or suspended in the diluent and each of the one or more maintenance doses has a volume of 30 mL. In some embodiments, the diluent comprises a cerebral spinal fluid (CSF) sample from the human subject or an artificial cerebral spinal fluid (aCSF) solution. In some embodiments, the diluent comprises cerebral spinal fluid sample from the human subject.

In some embodiments, the pharmaceutical composition is administered into the intrathecal space of the human subject. In some embodiments, the pharmaceutical composition is administered into the cerebrospinal fluid of the human subject. In some embodiments, the pharmaceutical composition is administered into the brain of the human subject. In some embodiments, the pharmaceutical composition is administered into the cerebrospinal fluid in the brain of the human subject. In some embodiments, the pharmaceutical composition is administered as a bolus injection. In some embodiments, the method comprises administering the pharmaceutical composition as a bolus injection over 1 to 60 minutes, 1 to 50 minutes, 1 to 40 minutes, 1 to 30 minutes, 1 to 20 minutes, 1 to 10 minutes, 1 to 5 minutes, or 1 to 3 minutes. In some embodiments, the pharmaceutical composition is administered by infusion with a delivery pump. In some embodiments, the pharmaceutical composition is administered by intracerebroventricular injection. In some embodiments, the pharmaceutical composition is administered by intrathecal injection. In some embodiments, the pharmaceutical composition is administered by lumbar injection.

In some embodiments, the compound is solubilized or diluted in an isotonic solution. In some embodiments, the compound is solubilized or diluted in a phosphate-buffered solution with at least pH 5.8. In some embodiments, the compound is solubilized or diluted in a phosphate-buffered (pH 6.6-7.6) solution. In some embodiments, the compound is solubilized or diluted in a buffer comprising 25-250 mM NaCl. In some embodiments, the compound is solubilized or diluted in a buffer comprising 0.1-20 mM KCl. In some embodiments, the compound is solubilized or diluted in a buffer comprising 0.1-50 mM Na₂HPO₄. In some embodiments, the compound is solubilized or diluted in a buffer comprising 0.1-50 mM NaH₂PO₄. In some embodiments, the compound is solubilized or diluted in a buffer comprising 0.1-50 mM CaCl₂. In some embodiments, the compound is solubilized or diluted in a buffer comprising 0.1-50 mM MgCl₂. In some embodiments, the compound is solubilized or diluted in a buffer comprising 150 mM NaCl, 3.0 mM KCl, 0.7 mM Na₂HPO₄, 0.3 mM NaH₂PO₄, 0.79 mM MgCl₂, and 1.4 mM CaCl₂. In some embodiments, the compound is solubilized or diluted in a buffer further comprising 1-100 mM NaHCO₃, 1-100 mM KHCO₃, or a combination thereof. In some embodiments, the compound is solubilized or diluted in a buffer further comprising carbohydrates. In some embodiments, the carbohydrates comprise D-glucose. In some embodiments, the compound is solubilized or diluted in a buffer further comprising 1-100 mM D-glucose. In some embodiments, the compound is solubilized or diluted in a buffer further comprising an antioxidant. In some embodiments, the antioxidant is t-butylhydroxyquinoline (TBHQ), butylated hydroxytoluene (BHT), butylated hydroxyanisole (BHA), vitamin E, or any combination thereof. In some embodiments, the pharmaceutical formulation does not comprise a preservative.

In some embodiments, the compound is present in the pharmaceutical composition at a concentration of from 0.1 mg/mL to 100 mg/mL. In some embodiments, the compound is present in the pharmaceutical composition at a concentration of about 0.1 mg/mL, 0.5 mg/mL, 1 mg/mL, 2 mg/mL, 2.5 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 22.5 mg/mL, or 25 mg/mL.

In some embodiments, the human subject is at most 18 years old at first dose. In some embodiments, the human subject is from 1 to 18, from 2 to 18, from 3 to 18, from 4 to 18, from 5 to 18, from 6 to 18, from 7 to 18, from 8 to 18, from 9 to 18, from 10 to 18, from 11 to 18, from 12 to 18, from 13 to 18, from 14 to 18, from 15 to 18, from 16 to 18, or from 17 to 18 years old. In some embodiments, the human subject is a human from 1 to 17, from 1 to 16, from 1 to 15, from 1 to 14, from 1 to 13, from 1 to 12, from 1 to 11, from 1 to 10, from 1 to 9, from 1 to 8, from 1 to 7, from 1 to 6, from 1 to 5, from 1 to 4, from 1 to 3, or from 1 to 2 years old. In some embodiments, the method treats the disease or condition.

In some embodiments, the disease or condition is Dravet Syndrome. In some embodiments, the human subject is characterized by having: seizure onset prior to 12 months of age with recurrent focal motor or hemiconvulsive or generalized tonic-clonic seizures, which are often prolonged and triggered by hyperthermia; no past history of causal magnetic resonance imaging lesion; no other known etiology of any diseases or conditions except Dravet Syndrome; normal development at seizure onset; a pathogenic variant, or variant of uncertain significance in an SCN1A gene; at least 2 prior treatments for epilepsy that either had lack of adequate seizure control; 4 or more convulsive seizures during the 28 days prior to administering, wherein the convulsive seizures is any one selected from Hemiclonic, Focal with Motor Signs, Focal to Bilateral Tonic Clonic Convulsion, Generalized Tonic Clonic Convulsion, Tonic, Tonic or Atonic (Drop Attacks), and Clonic; a current intervention for epilepsy or medication with at least one antiepileptic drug at a dose which has been stable for at least 4 weeks, wherein the interventions for epilepsy is a ketogenic diet, a vagal nerve stimulator, or a cannabinoid or marijuana-derived product; or any combination of (i)-(viii). In some embodiments, the human subject is additionally characterized by not having one or more of the following: one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly 1674Arg, and Asp 1866Tyr; a known pathogenic mutation in another gene that causes epilepsy, wherein the pathogenic mutation is homozygous in cases of known recessive disease; currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; clinically, significantly unstable medical conditions other than epilepsy; clinically, relevant symptoms or a clinically significant illness in the 4 weeks prior to administering, other than epilepsy: a history of brain or spinal cord disease other than epilepsy or Dravet Syndrome; or a history of bacterial meningitis or brain malformation; a spinal deformity or other condition that alters the free flow of cerebrospinal fluid (CSF) or having an implanted CSF drainage shunt; clinically significant abnormal laboratory values prior to administering; aspartate aminotransferase or alanine aminotransferase >2.5-fold upper limit of normal, serum creatinine greater than an upper limit of normal or platelet count less than a lower limit of normal; clinically relevant abnormalities in the 12-lead electrocardiogram (ECG) measured at prior to administering; a psychiatric or behavioral disorder; currently or in the past 4 weeks, medication of an anticoagulant, wherein the anticoagulant is not aspirin; or any combination of (a)-(l).

In some embodiments, the human subject comprises a missense or a nonsense mutation in SCN1A gene. In some embodiments, the method reduces or ameliorates at least one symptom of Dravet Syndrome in the human subject. In some embodiments, the symptom of Dravet Syndrome is a seizure. In some embodiments, the administration reduces or ameliorates seizure frequency, seizure intensity, or seizure duration. In some embodiments, the method further comprises assessing tolerability or effectiveness of the pharmaceutical composition.

In some embodiments, the method further comprises administrating at least one additional therapeutic agent or therapy. In some embodiments, the at least one additional therapeutic agent or therapy is administered at the same time as the first dose. In some embodiments, the at least one additional therapeutic agent or therapy is administered at the same time as the one or more maintenance doses. In some embodiments, the at least one additional therapeutic agent or therapy is administered prior to administration of the first dose. In some embodiments, the at least one additional therapeutic agent or therapy is administered after administration of the first dose. In some embodiments, the at least one additional therapeutic agent or therapy comprises fenfluramine.

In some embodiments, the compound reduces expression or function of Na_{V}1.1 protein that is associated with an altered splicing of a nonsense-mediated RNA decay-inducing exon from a pre-mRNA that contains the NMD exon and encodes Na_{V}1.1 protein. In some embodiments, the compound promotes exclusion of the NMD exon from the pre-mRNA that contains the NMD exon and that encodes the Na_{V}1.1 protein. In some embodiments, the compound binds to a targeted portion of a pre-mRNA that contains the NMD exon and that encodes the Na_{V}1.1 protein. In some embodiments, the compound increases a level of processed mRNA encoding the Na_{V}1.1 protein when the ASO is introduced into the cell. In some embodiments, the compound increases a level of the Na_{V}1.1 protein when the compound is introduced into the cell. In some embodiments, the targeted portion is within an intron sequence flanking the NMD exon. In some embodiments, the targeted portion comprises at least one nucleotide of the NMD exon. In some embodiments, the targeted portion is within the NMD exon. In some embodiments, the brain concentration of the compound after administration of the pharmaceutical composition is at least 1, 2, 4, 6, 8, or 10 μg/ml.

### INCORPORATION BY REFERENCE

All publications, patents, and patent applications mentioned in this specification are herein incorporated by reference to the same extent as if each individual publication, patent, or patent application is specifically and individually indicated to be incorporated by reference.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B depict schematic representations of a target pre-mRNA that contains a nonsense-mediated RNA decay-inducing exon (NMD exon mRNA) and therapeutic agent-mediated exclusion of the nonsense-mediated mRNA decay-inducing exon from the pre-mRNA to increase expression of the full-length target protein or functional RNA. FIG. 1A shows a cell divided into nuclear and cytoplasmic compartments. In the nucleus, a pre-mRNA transcript of a target gene undergoes splicing to generate processed mRNA, and this processed mRNA is exported to the cytoplasm and translated into target protein. For this exemplary target gene, some fraction of the processed mRNA contains a nonsense-mediated mRNA decay-inducing exon (NMD exon mRNA) that is degraded in the cytoplasm, thus leading to no target protein production. FIG. 1B shows an example of the same cell divided into nuclear and cytoplasmic compartments. Treatment with a therapeutic agent, such as an antisense oligomer (ASO), promotes the exclusion of the nonsense-mediated mRNA decay-inducing exon from the pre-mRNA and results in an increase in processed mRNA, which is in turn translated into higher levels of target protein.

FIG. 1C is a schematic representation of therapeutic ASO-mediated exclusion of a nonsense-mediated mRNA decay-inducing exon from a pre-mRNA, which decreases non-productive processed mRNA (e.g., with an NMD exon) and increases productive mRNA (e.g., without an NMD exon) and increases expression of the full-length target protein from the productive mRNA.

FIG. 1D shows identification of an exemplary sequence in the SCN1A gene that encodes a nonsense-mediated mRNA decay (NMD)-inducing exon. The identification of the sequence in the SCN1A gene that encodes the NMD-inducing exon using comparative genomics is shown, visualized in the UCSC genome browser. The upper panel shows a graphic representation of the SCN1A gene to scale. The conservation level across 100 vertebrate species is shown as peaks. The highest peaks correspond to exons (black boxes), while no peaks are observed for the majority of the introns (lines with arrow heads). Peaks of conservation were identified in intron 20 (NM_006920), shown in the middle panel. Inspection of the conserved sequences identified an exon-like sequence of 64 bp (bottom panel, sequence highlighted in grey) flanked by 3′ and 5′ splice sites (underlined sequence). Inclusion of this exon leads to a frameshift and the introduction of a premature termination codon in exon 21 rendering the transcript a target of NMD. Figure discloses SEQ ID NO: 1101.

FIG. 2 shows a study design timeline for monitoring wild-type (WT) and Dravet Syndrome (DS) mice as well as a Kaplan-Meier curve showing DS and WT littermate mice monitored to 14 weeks for survival.

FIG. 3 shows an experimental design for the EEG seizure monitoring study in DS mice and their WT littermates.

FIGS. 4A-4E show the results of monitoring seizures in mice administered with ASO-22 or phosphate buffered saline (PBS). FIG. 4A shows exemplary EEG recordings in DS mice. FIG. 4B shows the number of seizures occurring in various regions of the brain in the two mice groups. * Indicates p<0.05. FIG. 4C summarizes the total number of spontaneous seizures (generalized and focal) recorded between P22 and P46 in DS mice dosed with PBS (n=21) or ASO-22 (n=21). * Indicates p<0.05. FIG. 4D shows the number of mice that had a number of seizures in each group. FIG. 4E shows the effect of ASO-22 on the latency to the first recorded seizure between P22 and P46 in DS mice dosed with PBS (n=21) or ASO-22 (n=21).

FIGS. 5A-5G show that a single ICV injection of 20 μg ASO-22 at P2 results in reduced sudden unexpected death in epilepsy (SUDEP) incidence and increased Na_{V}1.1 protein expression in DS mice. FIG. 5A is a schematic for the experimental design. FIGS. 5B, 5C, 5D, 5E, 5F, and 5G illustrate the ASO-22 exposure, Scn1a expression, and Na_{V}1.1 expression in brain tissues at 7 weeks or 14 weeks after a single ICV injection of ASO-22 (20 μg) or PBS on P2, respectively.

FIGS. 6A-6B show the percent survival of DS and WT mice after a single ICV injection of ASO-22 (60 μg) or PBS on P14. FIG. 6A is a schematic showing the study design. FIG. 6B is a line chart showing the percent survival (y-axis) of the mice in various conditions (wild-type mice treated with PBS; wild-type mice treated with ASO-22; Scn1a^{+/-} mice treated with ASO-22; and Scn1a^{+/-} mice treated with PBS) over the course of days (x-axis).

FIGS. 7A-7F show the ASO-22 exposure, Scn1a expression, and Na_{V}1.1 expression in brain tissues at P35 and P90 after a single ICV injection of ASO-22 (60 μg) or PBS on P14, respectively. FIG. 7A illustrates the concentration of ASO-22 in brain tissue (μg/g), FIG. 7B shows the fold change of Scn1a gene expression, and FIG. 7C shows the Na_{V}1.1 expression relative to adult brain tissue at P35 for wild-type and Scn1a^{+/-} mice after ICV injection with either ASO-22 (60 μg) or PBS on P14. FIG. 7D illustrates the concentration of ASO-22 in brain tissue (μg/g), FIG. 7E shows the fold change of Scn1a gene expression, and FIG. 7F shows the Na_{V}1.1 expression relative to adult brain tissue at P90 for wild-type and Scn1a^{+/-} mice after ICV injection with either ASO-22 (60 μg) or PBS on P14.

FIG. 8 shows the experimental conditions and numbers of monkeys used per group.

FIGS. 9A-9B show the levels of ASO-22 in the cynomolgus monkey brain on study day 3 (FIG. 9A) and day 29 (FIG. 9B).

FIGS. 10A-10B show the levels of Na_{V}1.1 protein in cynomolgus monkey brain regions on study day 3 (FIG. 10A) and day 29 (FIG. 10B).

FIGS. 11A-11B show the percentages of productive SCN1A gene to total SCN1A gene as an evaluation of target engagement in cynomolgus monkeys on study day 3 (FIG. 11A) and day 29 (FIG. 11B).

FIG. 12A shows the plasma pharmacokinetics in cynomolgus monkey after Intrathecal Administration of ASO-22. FIG. 12B shows the levels of ASO-22 in the cynomolgus monkey cerebrospinal fluid (CSF) on study day 3 and 29.

FIGS. 13A-13D depict identification of an alternative splicing event in SCN1A that results in NMD. FIG. 13A shows SCN1A splicing isoforms with or without inclusion of the alternative exon in ReNcells as demonstrated by RT-PCR. FIG. 13B shows evaluation of the alternative splice event of the SCN1A gene in the cerebral cortex from 4 species. FIG. 13C shows an image of a TBE PAGE gel of RT-PCR products corresponding to Scn1a productive (lower bands, 498 bp) and non-productive transcript (upper bands, 562 bp) amplified from total RNA extracted from WT C57BL/6J mouse brains from P0 to P20 and at 10 months. Mouse Gapdh was used as a loading control. FIG. 13D summarizes expression of Scn1a productive and non-productive transcript in postnatal mouse brains, calculated with optical densities of PCR products shown in FIG. 13C.

FIGS. 14A-14E depict that selected ASOs suppressed the NMD splicing event and increased the expression of productive SCN1A mRNA in ReNcells. FIG. 14A is an exemplary TBE PAGE gel image of RT-PCR products corresponding to SCN1A productive (lower bands, 549 bp) and non-productive mRNA containing exon 20X (upper bands, 613 bp) in ReNcells after gymnotic (free) uptake of ASO. FIG. 14B is a histogram showing the percentage of Exon 20X inclusion (y-axis) after treatment with various ASOs (x-axis). FIG. 14C is a histogram showing the fold change in Scn1a gene expression (y-axis) after treatment with various ASOs (x-axis). FIG. 14D is a graph illustrating the fold change in Scn1a gene expression (y-axis) after treatment with ASO-22 at various concentrations in nM (x-axis) by free uptake or nucleofection. FIG. 14E is a histogram showing the fold change in Scn1a gene expression (y-axis) after treatment with either a non-targeting ASO (NT) or ASO-22, each at various concentrations (20 μM, 8 μM, 3 μM) (x-axis).

FIGS. 15A-15C show dose-dependent effects of ASO-22 on splicing and expression of SCN1A mRNA in ReNcells. FIG. 15A shows an exemplary TBE PAGE gel image of RT-PCR products corresponding to SCN1A productive mRNA (lower bands, 549 bp) and non-productive mRNA containing exon 20X (upper bands, 613 bp) in ReNcells. RPL32 was used as the loading control. FIG. 15B is a histogram showing the fold change in SCN1A productive mRNA after treatment with various controls (sham, sham with cycloheximide, non-targeting (NT) ASO, or NT ASO with cycloheximide), ASO-22 at various concentrations (20 μM, 8 μM, 3 μM), or ASO-22 with cycloheximide at various concentrations (20 μM, 8 μM, 3 μM) as indicated on the x-axis. FIG. 15C is a histogram showing the fold change in SCN1A non-productive mRNA after treatment with various controls (sham, sham with cycloheximide, non-targeting (NT) ASO, or NT ASO with cycloheximide), ASO-22 at various concentrations (20 μM, 8 μM, 3 μM), or ASO-22 with cycloheximide at various concentrations (20 μM, 8 μM, 3 μM) as indicated on the x-axis.

FIGS. 16A-16H depict ASO-22 ICV injection causes dose-dependent and durable increases in Scn1a mRNA and Na_{V}1.1 protein expression in mouse brain. FIG. 16A is a schematic illustrating the experimental study design for ASO-22. FIG. 16B is a graph showing the percentage of Exon 21X inclusion (amount of non-productive Scn1a transcript) after a single ICV injection of ASO-22 at 0.3, 1, 3, 5, 10, 20 or 30 μg (x-axis). FIG. 16C is a graph showing the fold change in mRNA (amount of productive Scn1a transcript) after a single ICV injection of ASO-22 at 0.3, 1, 3, 5, 10, 20 or 30 μg (x-axis). FIG. 16D is a graph showing the percentage of Na_{V}1.1 protein expression relative to adult brain tissue after a single ICV injection of ASO-22 at 0.3, 1, 3, 5, 10, 20 or 30 μg (x-axis). FIG. 16E is a histogram showing the effect of ASO-22 (fold change in mRNA) on expression of the 9 VGSC α subunit genes in mouse brain. Expression of Scn1a productive transcript and the remaining 8 VGSC α subunit genes plus Nax (Scn7a) in mouse brains following ICV injection of PBS, a non-target ASO control (NT, 20 μg) or different doses of ASO-22 was measured by probe-based qPCR. Expression of each transcript was first normalized to endogenous Gapdh then compared to PBS injection controls. FIG. 16F is a schematic of the experimental study design used to determine the durability of the ASO-22 effect in brain. FIG. 16G shows the relative expression of productive Scn1a transcript (y-axis) after treatment with ASO-22 or PBS over the course of days (x-axis). FIG. 16H shows the percentage of Na_{V}1.1 protein expression relative to adult brain tissue (y-axis) after treatment with ASO-22 or PBS over the course of days (x-axis).

FIG. 17 shows dose-dependent effects of ASO-22 on expression of Scn1a mRNA in ICV-injected neonatal mouse brains (§ =nonproductive; *=productive).

FIG. 18 shows dose-dependent effects of ASO-22 on expression of Na_{V}1.1 in ICV-injected neonatal mouse brains.

FIG. 19 shows expression of Scn1a mRNA in mouse brains at different post-injection days (§ =nonproductive; *=productive).

FIG. 20 shows expression of Na_{V}1.1 in mouse brains at different post-injection days.

FIG. 21 shows validation of the two anti-Na_{V}1.1 antibodies used in the Examples. Specificity of the two anti-Na_{V}1.1 antibodies, Alomone ASC-001 and NeuroMab 75-023, was tested using total protein prepared from a Scn1a^{31/-} mouse brain (middle lane) and brains of two WT littermates (left and right lanes).

FIG. 22 shows a schematic representation of clinical manifestations of Dravet Syndrome and their relative incidences according to age. AA: atypical absences; AE: acute encephalopathy; CG: crouching gait; CPS: complex partial seizures; DD: developmental delay; DS: Dravet syndrome; EEG: electroencephalogram; FSz: complex febrile seizures; GMS: generalized motor seizures; HS: hyperthermia sensitivity; m: month; MSz: myoclonic seizures; OS: obtundation status; SE: status epilepticus; SUDEP: sudden unexpected death in epilepsy; y: years; * Moderate fever for 60%, mostly clonic generalized and unilateral motor seizures; ** Difficult to distinguish between AA and CPS without ictal EEG recording, so their precise incidence is unknown. See, e.g., Gataullina and Dulac, 2017, of which the entire content is incorporated herein by reference.

FIG. 23 shows TANGO (Targeted Augmentation of Nuclear Gene Output) that may be used to treat Dravet syndrome.

FIG. 24 shows the transformative potential of TANGO technology in Dravet syndrome.

FIG. 25 shows the study design. Phase 1/2a open-label, 2-part study conducted at approximately 20 sites in the United States.

FIG. 26 shows a schematic representation of the study design.

FIG. 27 shows patient inclusion and exclusion criteria.

FIG. 28 shows study assessments.

FIG. 29 shows workflow of a Phase 1/2a study of Compound-A.

FIG. 30 shows two plots summarizing cerebrospinal fluid (CSF) exposure of Compound-A in single ascending dose (SAD) cohorts and multiple ascending dose (MAD) cohorts, respectively.

FIGS. 31A-31B show two plots summarizing mean percent change in seizure frequency from baseline level to the period between Day 29 and 84 post administration of Compound-A (in reference to 1^{st} dose, Day 29-84) in 2- to 12-year-old subjects (FIG. 31A) and 13- to 18-year-old (FIG. 31B), all combined by cohort.

FIGS. 32A-32B show two plots summarizing mean percent change in seizure frequency from baseline in patients of all ages as combined by cohort in the period between Day 1 and Day 84 (Day 1-84) post administration (FIG. 32A), and in the period between Day 29-84 (Day 29-84) post administration (FIG. 32B).

FIGS. 33A-33C show the study design for the Phase 1/2a clinical trials for ASO-1. FIG. 33A shows the study design and dosing schedule using the Single Ascending Dose (SAD) regime (MONARCH) before patients can become eligible to enroll in the LONGWING OLE. FIG. 33B shows the study design and dosing schedule using the Multiple Ascending Dose regime (MONARCH) before patients can become eligible to enroll in the LONGWING OLE. FIG. 33C shows the study design and dosing schedule using the Multiple Ascending Dose regime (ADMIRAL) before patients can become eligible to enroll in the LONGWING OLE.

FIG. 34 shows a plot summarizing mean percent change in seizure frequency from baseline in patients of all ages receiving 30 mg, 45 mg of ASO-1 as combined by cohort in the period between Day 1 and Day 225 post administration in the MONARCH study and the mean percent change in seizure frequency from baseline in patients of all ages receiving 30 mg, 45 mg, or 70 mg of ASO-1 as combined by cohort in the period between Day 1 and Day 225 post administration in the ADMIRAL study.

FIG. 35 shows a plot summarizing mean percent change in seizure frequency from baseline in patients of all ages receiving 30 mg or 45 mg of ASO-1 as combined by cohort in the period between Day 29 and Day 85 post administration in the MONARCH study and the mean percent change in seizure frequency from baseline in patients of all ages receiving 30 mg, 45 mg, or 70 mg of ASO-1 as combined by cohort in the period between Day 29 and Day 87 post administration in the ADMIRAL study.

FIGS. 36A-36B show plots summarizing mean percent change in seizure frequency from baseline in patients between ages 2-12 and between ages 13-18 years receiving 30 mg or 45 mg of ASO-1 as combined by cohort in the period between Day 1 and Day 225 post administration in the MONARCH study. FIG. 36A shows a plot summarizing mean percent change in seizure frequency from baseline in patients between ages 2-12 and between ages 13-18 years receiving 30 mg of ASO-1 as combined by cohort in the period between Day 1 and Day 225 post administration in the MONARCH study. FIG. 36B shows a plot summarizing mean percent change in seizure frequency from baseline in patients between ages 2-12 and between ages 13-18 years receiving 45 mg of ASO-1 as combined by cohort in the period between Day 1 and Day 225 post administration in the MONARCH study.

FIGS. 37A-37C show plots summarizing mean percent change in seizure frequency from baseline in patients between ages 2-12 and between ages 13-18 years receiving 30 mg, 45 mg, or 70 mg of ASO-1 as combined by cohort in the period between Day 1 and Day 252 post administration in the ADMIRAL study. FIG. 37A shows a plot summarizing mean percent change in seizure frequency from baseline in patients between ages 2-12 and between ages 13-18 years receiving 30 mg of ASO-1 as combined by cohort in the period between Day 1 and Day 252 post administration in the ADMIRAL study. FIG. 37B shows a plot summarizing mean percent change in seizure frequency from baseline in patients between ages 2-12 and between ages 13-18 years receiving 45 mg of ASO-1 as combined by cohort in the period between Day 1 and Day 252 post administration in the ADMIRAL study. FIG. 37C shows a plot summarizing mean percent change in seizure frequency from baseline in patients between ages 2-12 and between ages 13-18 years receiving 70 mg of ASO-1 as combined by cohort in the period between Day 1 and Day 252 post administration in the ADMIRAL study.

FIG. 38 shows a plot summarizing mean percent change in seizure frequency from baseline in patients between ages 2-12 and between ages 13-18 years receiving 2 or 3 doses of 70 mg of ASO-1, as combined by cohort in the period between Day 1 and Day 252 post administration in the ADMIRAL study.

FIGS. 39A-39B show plots summarizing mean percent change in seizure frequency from baseline in all patients receiving 30 mg, 45 mg, or 70 mg of ASO-1, as analyzed 3 or 6 months post last dose administration. FIG. 39A shows a plot summarizing mean percent change in seizure frequency from baseline in all patients receiving 30 mg, 45 mg, or 70 mg of ASO-1, as analyzed 3 months post last dose administration. FIG. 39B shows a plot summarizing mean percent change in seizure frequency from baseline in all patients receiving 30 mg, 45 mg, or 70 mg of ASO-1, as analyzed 6 months post last dose administration.

FIGS. 40A-40B show plots summarizing mean percent change in seizure frequency from baseline in all patients receiving 2 or 3 doses of 70 mg of ASO-1, as analyzed 3 or 6 months post last dose administration in the ADMIRAL study. FIG. 40A shows a plot summarizing mean percent change in seizure frequency from baseline in all patients receiving 2 or 3 doses of 70 mg of ASO-1, as analyzed 3 months post last dose administration in the ADMIRAL study. FIG. 40B shows a plot summarizing mean percent change in seizure frequency from baseline in all patients receiving 2 or 3 doses of 70 mg of ASO-1, as analyzed 6 months post last dose administration in the ADMIRAL study.

FIG. 41 shows a plot summarizing mean percent change in seizure frequency from baseline in all patients receiving 30 mg or 45 mg of ASO-1 maintenance dose every 4 months in the OLE study.

FIGS. 42A-42B show plots showing improvement in receptive communication and expressive communication in patients receiving ASO-1 for over one year of dosing compared to observed natural history of the disease measured on the Vineland (VABS-III) scale. FIG. 42A shows a plot showing improvement in receptive communication in patients receiving ASO-1 for over one year of dosing compared to observed natural history of the disease measured on the Vineland (VABS-III) scale. FIG. 42B shows a plot showing improvement in receptive communication in patients receiving ASO-1 for over one year of dosing compared to observed natural history of the disease measured on the Vineland (VABS-III) scale.

FIG. 43 shows a plot showing improvement in gross motor skills in patients receiving ASO-1 for over one year of dosing compared to observed natural history of the disease measured on the Vineland (VABS-III) scale.

FIG. 44 shows a plot showing improvement in executive function in patients receiving ASO-1 for over one year of dosing compared to observed natural history of the disease measured on the Vineland (VABS-III) scale.

FIGS. 45A-45B show plots showing improvement in clinical and caregiver impression of change in patients receiving ASO-1 for over one year of dosing compared to observed natural history of the disease measured on the Vineland (VABS-III) scale. FIG. 45A shows a plot showing improvement in clinical impression of change in patients receiving ASO-1 for over one year of dosing compared to observed natural history of the disease measured on the Vineland (VABS-III) scale. FIG. 45B shows a plot showing improvement in caregiver impression of change in patients receiving ASO-1 for over one year of dosing compared to observed natural history of the disease measured on the Vineland (VABS-III) scale.

### DETAILED DESCRIPTION

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below.

### Definitions

As used in this specification and the appended claims, the singular forms “a,” “an,” and “the” include plural referents unless the content clearly dictates otherwise.

It should be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated, the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “embodiments,” “some embodiments,” “an embodiment,” “one embodiment,” “certain embodiments,” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

The terms “oligonucleotide sequence,” “nucleic acid sequence,” “polynucleic acid sequence,” “nucleotide sequence,” and “nucleotide acid sequence” are used herein interchangeably in its broadest sense and have the identical meaning herein and refer to preferably DNA or RNA. A nucleic acid sequence is a polymer comprising or consisting of nucleotide monomers, which are covalently linked to each other by phosphodiester-bonds of a sugar/phosphate-backbone. The term “nucleic acid sequence” also encompasses modified nucleic acid sequences, such as base-modified, sugar-modified or backbone-modified, etc., DNA or RNA.

The term “fragment,” or “fragment of a sequence,” which have the identical meaning herein, is a shorter portion of a full-length sequence of e.g., a nucleic acid molecule like DNA or RNA or a protein. Accordingly, a fragment, typically, consists of a sequence that is identical to the corresponding stretch within the full-length sequence. A preferred fragment of a sequence in the context of the present invention consists of a continuous stretch of entities, such as nucleotides or amino acids corresponding to a continuous stretch of entities in the molecule the fragment is derived from, which represents at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, or even 100% of the total (i.e., full-length) molecule from which the fragment is derived. For example, a “fragment” or “functional fragment” of a polynucleotide or a polypeptide is a fragment of the polynucleotide or the polypeptide that is shorter than the full-length, immature, or mature nucleotide or polypeptide and has at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99%, at least 99.5%, or even 100% or more of the activity of the full-length mature reference polynucleotide or polypeptide. Fragments of interest can be made by recombinant, synthetic, or digestive methods.

The term “recombinant” when used with reference, for example, to a cell, a nucleic acid, a protein, or a vector, indicates that the cell, nucleic acid, protein, or vector has been modified by or is the result of laboratory methods. Thus, for example, the term “recombinant polynucleotide” can refer to a polynucleotide that is not naturally occurring and are synthesized or manipulated in vitro, such as polynucleotides produced by laboratory methods. A recombinant polynucleotide can be synthesized in a laboratory and/or can be prepared by using recombinant DNA technology by using enzymatic modification of DNA, such as enzymatic restriction digestion, ligation, and cloning. A recombinant polypeptide can be prepared by in vitro transcription of a recombinant DNA followed by in vitro translation of the produced messenger RNA (mRNA). Alternatively, under suitable conditions, a recombinant polynucleic acid or RNA can be incorporated into a cell and a recombinant polypeptide can be expressed within the cell. Recombinant proteins can include amino acid residues not found within the native (non-recombinant) form of the protein or can be include amino acid residues that have been modified, e.g., labeled.

The term “isolated” means separated from constituents, cellular and otherwise, in which the polynucleotide, polypeptide, protein, or fragments thereof, are normally associated with in nature. For example, with respect to a polynucleotide, an isolated polynucleotide is one that is separated from the 5′ and 3′ ends with which it is normally associated in the naturally occurring sequence. As is apparent to those of skill in the art, a non-naturally occurring polynucleotide, polypeptide, protein, or fragments thereof, does not require “isolation” to distinguish it from its naturally occurring counterpart. In addition, a “concentrated”, “separated” or “diluted” polynucleotide, polypeptide, protein, or fragments thereof, is distinguishable from its naturally occurring counterpart in that the concentration or number of molecules per volume is greater than “concentrated” or less than “separated” or “diluted” than that of its naturally occurring counterpart.

As used herein, “nucleotide” means a nucleoside further comprising a phosphate linking group. As used herein, “linked nucleosides” may or may not be linked by phosphate linkages and thus includes, but is not limited to, “linked nucleotides.” As used herein, “linked nucleosides” are nucleosides that are connected in a continuous sequence (i.e., no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety.

As used herein, “naturally occurring sugar moiety” means a ribofuranosyl as found in naturally occurring RNA or a deoxyribofuranosyl as found in naturally occurring DNA.

As used herein, “sugar moiety” means a naturally occurring sugar moiety or a modified sugar moiety of a nucleoside.

As used herein, “modified sugar moiety” means a substituted sugar moiety, a bicyclic or tricyclic sugar moiety, or a sugar surrogate.

The terms “identical” or percent “identity,” in the context of two or more nucleic acids or polypeptide sequences, refer to two or more sequences or subsequences that are the same or have a specified percentage of nucleotides or amino acid residues that are the same (i.e., 30%, 35%, 40%, 45%, 50%, 55%, 60%, 65%, 70%, 75%, 80%, 85%, 90%, 95%, 98%, 99%, 99.5%, 99.8%, 99.9%, or 100% identity over a specified region, e.g., of the entire polypeptide sequences of the invention or individual domains of the polypeptides of the invention), when compared and aligned for maximum correspondence over a comparison window, or designated region as measured using a sequence comparison algorithm or by manual alignment and visual inspection. Such sequences that are at least about 80% identical are said to be “substantially identical.” In some embodiments, two sequences are 100% identical. In some embodiments, two sequences are 100% identical over the entire length of one of the sequences (e.g., the shorter of the two sequences where the sequences have different lengths). In various embodiments, identity may refer to the complement of a test sequence.

In some embodiments, the identity exists over a region that is at least about 2 to about 400 amino acids or nucleotides in length. In some embodiments, the identity exists over a region that is at least about 2 to about 390, at least about 2 to about 380, at least about 2 to about 370, at least about 2 to about 360, at least about 2 to about 350, at least about 2 to about 340, at least about 2 to about 330, at least about 2 to about 320, at least about 2 to about 310, at least about 2 to about 300, at least about 2 to about 290, at least about 2 to about 280, at least about 2 to about 270, at least about 2 to about 260, at least about 2 to about 250, at least about 2 to about 200, at least about 2 to about 150, at least about 2 to about 100 amino acids or nucleotides in length. In some embodiments, the identity exists over a region that is at least about 2 to about 90, at least about 2 to about 85, at least about 2 to about 80, at least about 2 to about 75, at least about 2 to about 70, at least about 2 to about 65, at least about 2 to about 60, at least about 2 to about 55, at least about 2 to about 50, at least about 2 to about 45, at least about 2 to about 40, at least about 2 to about 35, at least about 2 to about 30, at least about 2 to about 25, at least about 2 to about 20, at least about 2 to about 10, at least about 2 to about 5 amino acids or nucleotides in length.

In some embodiments, the identity exists over a region that is at least about 3 to about 400, about 4 to about 400, about 5 to about 400, about 6 to about 400, about 7 to about 400, about 8 to about 400, about 9 to about 400, about 10 to about 400, about 11 to about 400, about 12 to about 400, about 13 to about 400, about 14 to about 400, about 15 to about 400, about 16 to about 400, about 17 to about 400, about 18 to about 400, about 19 to about 400, about 20 to about 400, about 21 to about 400, about 22 to about 400, about 23 to about 400, about 24 to about 400, about 25 to about 400, about 26 to about 400, about 27 to about 400, about 28 to about 400, about 29 to about 400, about 30 to about 400, about 31 to about 400, about 32 to about 400, about 33 to about 400, about 34 to about 400, about 35 to about 400 amino acids or nucleotides in length. In some embodiments, the identity exists over a region that is at least about 40 to about 400, about 45 to about 400, about 50 to about 400, about 55 to about 400, about 60 to about 400, about 61 to about 400, about 62 to about 400, about 63 to about 400, about 64 to about 400, about 65 to about 400, about 66 to about 400, about 67 to about 400, about 68 to about 400, about 69 to about 400, about 70, to about 400, about 71 to about 400, about 72 to about 400, about 73 to about 400, about 74 to about 400, about 75 to about 400, about 80 to about 400, about 85 to about 400, about 90 to about 400, about 100 to about 400, about 150 to about 400, about 200 to about 400, about 250 to about 400, about 300 to about 400, about 350 to about 400 amino acids or nucleotides in length.

In some embodiments, the identity exists over a region that is at least about 2 to about 343, about 3 to about 343, about 4 to about 343, about 7 to about 343, about 9 to about 343, about 11 to about 343, about 15 to about 343, about 16 to about 343, about 20 to about 343, about 25 to about 343, about 62 to about 343, about 2 to about 317, about 3 to about 317, about 4 to about 317, about 7 to about 317, about 9 to about 317, about 11 to about 317, about 15 to about 317, about 16 to about 317, about 20 to about 317, about 25 to about 317, about 62 to about 317, about 2 to about 300, about 3 to about 300, about 4 to about 300, about 7 to about 300, about 9 to about 300, about 11 to about 300, about 15 to about 300, about 16 to about 300, about 20 to about 300, about 25 to about 300, about 62 to about 300, about 2 to about 62, about 3 to about 62, about 4 to about 62, about 7 to about 62, about 9 to about 62, about 11 to about 62, about 15 to about 62, about 16 to about 62, about 20 to about 62, about 25 to about 62 amino acids or nucleotides in length.

The term “genetically modified” means containing and/or expressing a foreign gene or nucleic acid sequence which in turn, modifies the genotype or phenotype of the cell or its progeny. In other words, it refers to any addition, deletion, or disruption to a cell's endogenous nucleotides.

The term “operably linked” can refer to a functional relationship between two or more nucleic acid sequences, e.g., a functional relationship of a transcriptional regulatory or signal sequence to a transcribed sequence. For example, a target motif or a nucleic acid encoding a target motif is operably linked to a coding sequence if it is expressed as a preprotein that participates in targeting the polypeptide encoded by the coding sequence to a cell membrane, intracellular, or an extracellular compartment. For example, a signal peptide or a nucleic acid encoding a signal peptide is operably linked to a coding sequence if it is expressed as a preprotein that participates in the secretion of the polypeptide encoded by the coding sequence. For example, a promoter is operably linked if it stimulates or modulates the transcription of the coding sequence.

The term “subject” or “patient” encompasses vertebrates or mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice, guinea pigs, and the like. In one aspect, the mammal is a human. The term “animal” as used herein comprises human beings and non-human animals. In one embodiment, a “non-human animal” is a mammal, for example, a rodent such as rat or a mouse. In one embodiment, a non-human animal is a mouse.

A “control” is an alternative subject or sample used in an experiment for comparison purpose. A control can be “positive” or “negative.”

### Methods of Treatment

In some aspects, provided herein is a method of treating or preventing a disease or condition characterized by a reduced expression or function of Na_{V}1.1 protein in a human subject in need thereof comprising administering to the human subject a pharmaceutical composition comprising a compound at a first dose of about 0.1, 0.5, 1, 2.5, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mg, wherein the compound is an antisense oligomer (ASO) that comprises a sequence with at least 80% sequence identity to any one of SEQ ID NOs: 21-67, 210-256 or 304-1099, thereby treating or preventing the disease or condition in the human subject. In some embodiments, the ASO comprises a sequence with at least 80% sequence identity to any one of the sequences listed in Tables 4A, 4B, 5A, 5B, 6A, 6B, 7, 8A, and 8B, thereby treating or preventing the disease or condition in the human subject.

In some aspects, provided herein is a method of treating or preventing a disease or condition characterized by a reduced expression or function of Na_{V}1.1 protein in a human subject in need thereof comprising administering to the human subject a pharmaceutical composition comprising a first dose of a compound, wherein the compound is an ASO that comprises a sequence with at least 80% sequence identity to any one of SEQ ID NOs: 21-67, 210-256 or 304-1099, thereby treating or preventing the disease or condition in the human subject; wherein the human subject is at most 18 years old. In some embodiments, the ASO comprises a sequence with at least 80% sequence identity to any one of the sequences listed in listed in Tables 4A, 4B, 5A, 5B, 6A, 6B, 7, 8A, and 8B, thereby treating or preventing the disease or condition in the human subject; wherein the human subject is at most 18 years old.

In some aspects, provided herein is a method of treating or preventing a disease or condition characterized by a reduced expression or function of Na_{V}1.1 protein in a human subject in need thereof comprising administering to the human subject a pharmaceutical composition comprising a single dose of an antisense oligomer (ASO), wherein the ASO comprises a sequence with at least 80% sequence identity to any one of SEQ ID NOs: 21-67, 210-256 or 304-1099, thereby treating or preventing the disease or condition in the human subject. In some embodiments, the ASO comprises a sequence with at least 80% sequence identity to any one of any one of the sequences listed in listed in Tables 4A, 4B, 5A, 5B, 6A, 6B, 7, 8A, and 8B, thereby treating or preventing the disease or condition in the human subject.

In some embodiments, the pharmaceutical composition is administered into the intrathecal space of the human subject. In some embodiments, the pharmaceutical composition is administered into the cerebrospinal fluid of the human subject. In some embodiments, the pharmaceutical composition is administered into the brain of the human subject. In some embodiments, the pharmaceutical composition is administered into the cerebrospinal fluid in the brain of the human subject.

In some embodiments, the pharmaceutical composition is administered as a bolus injection. In some embodiments, the pharmaceutical composition is administered by infusion with a delivery pump. In some embodiments, the pharmaceutical composition is administered by intracerebroventricular injection. In some embodiments, the pharmaceutical composition is administered by intrathecal injection.

### Therapeutic Dose

In some embodiments, the first dose is a single dose. In some embodiments, the method further comprises assessing tolerability or effectiveness of the pharmaceutical composition.

In some embodiments, the method as described herein comprises administering to the human subject a pharmaceutical composition comprising a compound as described herein at a first dose of about 0.1, 0.5, 1, 2.5, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mg.

In some embodiments, the method as described herein comprises administering to the human subject a pharmaceutical composition comprising the compound as described herein at a first dose of from about 0.1 to about 1000 mg, from about 0.2 to about 1000 mg, from about 0.3 to about 1000 mg, from about 0.4 to about 1000 mg, from about 0.5 to about 1000 mg, from about 0.6 to about 1000 mg, from about 0.7 to about 1000 mg, from about 0.8 to about 1000 mg, from about 0.9 to about 1000 mg, 1 to about 1000 mg, from about 2 to about 1000 mg, from about 3 to about 1000 mg, from about 4 to about 1000 mg, from about 5 to about 1000 mg, from about 6 to about 1000 mg, from about 7 to about 1000 mg, from about 8 to about 1000 mg, from about 9 to about 1000 mg, from about 10 to about 1000 mg, from about 15 to about 1000 mg, from about 20 to about 1000 mg, from about 25 to about 1000 mg, from about 30 to about 1000 mg, from about 35 to about 1000 mg, from about 40 to about 1000 mg, from about 45 to about 1000 mg, from about 50 to about 1000 mg, from about 55 to about 1000 mg, from about 60 to about 1000 mg, from about 65 to about 1000 mg, from about 70 to about 1000 mg, from about 75 to about 1000 mg, from about 80 to about 1000 mg, from about 85 to about 1000 mg, from about 90 to about 1000 mg, from about 95 to about 1000 mg, from about 100 to about 1000 mg, from about 150 to about 1000 mg, from about 200 to about 1000 mg, from about 250 to about 1000 mg, from about 300 to about 1000 mg, from about 350 to about 1000 mg, from about 400 to about 1000 mg, from about 450 to about 1000 mg, from about 500 to about 1000 mg, from about 550 to about 1000 mg, from about 600 to about 1000 mg, from about 650 to about 1000 mg, from about 700 to about 1000 mg, from about 750 to about 1000 mg, from about 800 to about 1000 mg, from about 850 to about 1000 mg, from about 900 to about 1000 mg, or from about 950 to about 1000 mg.

In some embodiments, the method as described herein comprises administering to the human subject a pharmaceutical composition comprising the compound as described herein at a first dose of from 0.1 to 1000 mg, from 0.2 to 1000 mg, from 0.3 to 1000 mg, from 0.4 to 1000 mg, from 0.5 to 1000 mg, from 0.6 to 1000 mg, from 0.7 to 1000 mg, from 0.8 to 1000 mg, from 0.9 to 1000 mg, 1 to 1000 mg, from 2 to 1000 mg, from 3 to 1000 mg, from 4 to 1000 mg, from 5 to 1000 mg, from 6 to 1000 mg, from 7 to 1000 mg, from 8 to 1000 mg, from 9 to 1000 mg, from 10 to 1000 mg, from 15 to 1000 mg, from 20 to 1000 mg, from 25 to 1000 mg, from 30 to 1000 mg, from 35 to 1000 mg, from 40 to 1000 mg, from 45 to 1000 mg, from 50 to 1000 mg, from 55 to 1000 mg, from 60 to 1000 mg, from 65 to 1000 mg, from 70 to 1000 mg, from 75 to 1000 mg, from 80 to 1000 mg, from 85 to 1000 mg, from 90 to 1000 mg, from 95 to 1000 mg, from 100 to 1000 mg, from 150 to 1000 mg, from 200 to 1000 mg, from 250 to 1000 mg, from 300 to 1000 mg, from 350 to 1000 mg, from 400 to 1000 mg, from 450 to 1000 mg, from 500 to 1000 mg, from 550 to 1000 mg, from 600 to 1000 mg, from 650 to 1000 mg, from 700 to 1000 mg, from 750 to 1000 mg, from 800 to 1000 mg, from 850 to 1000 mg, from 900 to 1000 mg, or from 950 to 1000 mg.

In some embodiments, the method as described herein comprises administering to the human subject a pharmaceutical composition comprising the compound as described herein at a first dose of from about 0.1 to about 950 mg, from about 0.1 to about 900 mg, from about 0.1 to about 850 mg, from about 0.1 to about 800 mg, from about 0.1 to about 750 mg, from about 0.1 to about 700 mg, from about 0.1 to about 650 mg, from about 0.1 to about 600 mg, from about 0.1 to about 550 mg, from about 0.1 to about 500 mg, from about 0.1 to about 450 mg, from about 0.1 to about 400 mg, from about 0.1 to about 350 mg, from about 0.1 to about 300 mg, from about 0.1 to about 250 mg, from about 0.1 to about 200 mg, from about 0.1 to about 150 mg, from about 0.1 to about 100 mg, from about 0.1 to about 95 mg, from about 0.1 to about 90 mg, from about 0.1 to about 85 mg, from about 0.1 to about 80 mg, from about 0.1 to about 75 mg, from about 0.1 to about 70 mg, from about 0.1 to about 65 mg, from about 0.1 to about 60 mg, from about 0.1 to about 55 mg, from about 0.1 to about 50 mg, from about 0.1 to about 45 mg, from about 0.1 to about 40 mg, from about 0.1 to about 35 mg, from about 0.1 to about 30 mg, from about 0.1 to about mg, from about 0.1 to about 25 mg, from about 0.1 to about 20 mg, from about 0.1 to about 10 mg, from about 0.1 to about 9 mg, from about 0.1 to about 8 mg, from about 0.1 to about 7 mg, from about 0.1 to about 6 mg, from about 0.1 to about 5 mg, from about 0.1 to about 4 mg, from about 0.1 to about 3, from about 0.1 to about 2 mg, from about 0.1 to about 1 mg, from about 0.1 to about 0.9 mg, from about 0.1 to about 0.8 mg, from about 0.1 to about 0.7 mg, from about 0.1 to about 0.6 mg, from about 0.1 to about 0.5 mg, from about 0.1 to about 0.4 mg, from about 0.1 to about 0.3, or from about 0.1 to about 0.2 mg.

In some embodiments, the method as described herein comprises administering to the human subject a pharmaceutical composition comprising the compound as described herein at a first dose of from 0.1 to 950 mg, from 0.1 to 900 mg, from 0.1 to 850 mg, from 0.1 to 800 mg, from 0.1 to 750 mg, from 0.1 to 700 mg, from 0.1 to 650 mg, from 0.1 to 600 mg, from 0.1 to 550 mg, from 0.1 to 500 mg, from 0.1 to 450 mg, from 0.1 to 400 mg, from 0.1 to 350 mg, from 0.1 to 300 mg, from 0.1 to 250 mg, from 0.1 to 200 mg, from 0.1 to 150 mg, from 0.1 to 100 mg, from 0.1 to 95 mg, from 0.1 to 90 mg, from 0.1 to 85 mg, from 0.1 to 80 mg, from 0.1 to 75 mg, from 0.1 to 70 mg, from 0.1 to 65 mg, from 0.1 to 60 mg, from 0.1 to 55 mg, from 0.1 to 50 mg, from 0.1 to 45 mg, from 0.1 to 40 mg, from 0.1 to 35 mg, from 0.1 to 30 mg, from 0.1 to mg, from 0.1 to 25 mg, from 0.1 to 20 mg, from 0.1 to 10 mg, from 0.1 to 9 mg, from 0.1 to 8 mg, from 0.1 to 7 mg, from 0.1 to 6 mg, from 0.1 to 5 mg, from 0.1 to 4 mg, from 0.1 to 3, from 0.1 to 2 mg, from 0.1 to 1 mg, from 0.1 to 0.9 mg, from 0.1 to 0.8 mg, from 0.1 to 0.7 mg, from 0.1 to 0.6 mg, from 0.1 to 0.5 mg, from 0.1 to 0.4 mg, from 0.1 to 0.3, or from 0.1 to 0.2 mg.

In some embodiments, the method as described herein comprises administering to the human subject a pharmaceutical composition comprising the compound as described herein at a first dose of from about 1 to about 400 mg, from about 2 to about 400 mg, from about 3 to about 400 mg, from about 4 to about 400 mg, from about 5 to about 400 mg, from about 6 to about 400 mg, from about 7 to about 400 mg, from about 8 to about 400 mg, from about 9 to about 400 mg, from about 10 to about 400 mg, from about 20 to about 400 mg, from about 30 to about 400 mg, from about 40 to about 400 mg, from about 50 to about 400 mg, from about 60 to about 400 mg, from about 70 to about 400 mg, from about 80 to about 400 mg, from about 90 to about 400 mg, from about 100 to about 400 mg, from about 110 to about 400 mg, from about 120 to about 400 mg, from about 130 to about 400 mg, from about 140 to about 400 mg, from about 150 to about 400 mg, from about 160 to about 400 mg, from about 170 to about 400 mg, from about 180 to about 400 mg, from about 190 to about 400 mg, from about 200 to about 400 mg, from about 210 to about 400 mg, from about 220 to about 400 mg, from about 230 to about 400 mg, from about 240 to about 400 mg, from about 250 to about 400 mg, from about 260 to about 400 mg, from about 270 to about 400 mg, from about 280 to about 400 mg, from about 290 to about 400 mg, from about 300 to about 400 mg, from about 310 to about 400 mg, from about 320 to about 400 mg, from about 330 to about 400 mg, from about 340 to about 400 mg, from about 350 to about 400 mg, from about 360 to about 400 mg, from about 370 to about 400 mg, from about 380 to about 400 mg, or from about 390 to about 400 mg.

In some embodiments, the method as described herein comprises administering to the human subject a pharmaceutical composition comprising the compound as described herein at a first dose of from 1 to 400 mg, from 2 to 400 mg, from 3 to 400 mg, from 4 to 400 mg, from 5 to 400 mg, from 6 to 400 mg, from 7 to 400 mg, from 8 to 400 mg, from 9 to 400 mg, from 10 to 400 mg, from 20 to 400 mg, from 30 to 400 mg, from 40 to 400 mg, from 50 to 400 mg, from 60 to 400 mg, from 70 to 400 mg, from 80 to 400 mg, from 90 to 400 mg, from 100 to 400 mg, from 110 to 400 mg, from 120 to 400 mg, from 130 to 400 mg, from about 140 to 400 mg, from 150 to 400 mg, from about 160 to 400 mg, from 170 to 400 mg, from 180 to 400 mg, from 190 to 400 mg, from 200 to 400 mg, from 210 to 400 mg, from 220 to 400 mg, from 230 to 400 mg, from 240 to 400 mg, from 250 to 400 mg, from 260 to 400 mg, from 270 to 400 mg, from 280 to 400 mg, from 290 to 400 mg, from 300 to 400 mg, from 310 to 400 mg, from 320 to 400 mg, from 330 to 400 mg, from 340 to 400 mg, from 350 to 400 mg, from 360 to 400 mg, from 370 to 400 mg, from 380 to 400 mg, or from 390 to 400 mg.

In some embodiments, the method as described herein comprises administering to the human subject a pharmaceutical composition comprising the compound as described herein at a first dose of from about 10 to about 390 mg, from about 10 to about 380 mg, from about 10 to about 370 mg, from about 10 to about 360 mg, from about 10 to about 350 mg, from about 10 to about 340 mg, from about 10 to about 330 mg, from about 10 to about 320 mg, from about 10 to about 310 mg, from about 10 to about 300 mg, from about 10 to about 290 mg, from about 10 to about 280 mg, from about 10 to about 270 mg, from about 10 to about 260 mg, from about 10 to about 250 mg, from about 10 to about 240 mg, from about 10 to about 230 mg, from about 10 to about 220 mg, from about 10 to about 210 mg, from about 10 to about 200 mg, from about 10 to about 190 mg, from about 10 to about 180 mg, from about 10 to about 170 mg, from about 10 to about 160 mg, from about 10 to about 150 mg, from about 10 to about 140 mg, from about 10 to about 130 mg, from about 10 to about 120 mg, from about 10 to about 110 mg, from about 10 to about 90 mg, from about 10 to about 80 mg, from about 10 to about 70 mg, from about 10 to about 60 mg, from about 10 to about 50 mg, from about 10 to about 40 mg, from about 10 to about 30 mg, or from about 10 to about 20 mg.

In some embodiments, the method as described herein comprises administering to the human subject a pharmaceutical composition comprising the compound as described herein at a first dose of from 10 to 390 mg, from 10 to 380 mg, from 10 to 370 mg, from 10 to 360 mg, from 10 to 350 mg, from 10 to 340 mg, from 10 to 330 mg, from 10 to 320 mg, from 10 to 310 mg, from 10 to 300 mg, from 10 to 290 mg, from 10 to 280 mg, from 10 to 270 mg, from 10 to 260 mg, from 10 to 250 mg, from 10 to 240 mg, from 10 to 230 mg, from 10 to 220 mg, from 10 to 210 mg, from 10 to 200 mg, from 10 to 190 mg, from 10 to 180 mg, from 10 to 170 mg, from 10 to 160 mg, from 10 to 150 mg, from 10 to 140 mg, from 10 to 130 mg, from 10 to 120 mg, from 10 to 110 mg, from 10 to 90 mg, from 10 to 80 mg, from 10 to 70 mg, from 10 to 60 mg, from 10 to 50 mg, from 10 to 40 mg, from 10 to 30 mg, or from 10 to 20 mg.

In some embodiments, the method as described herein comprises administering to the human subject a pharmaceutical composition comprising the compound as described herein at a first dose of about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 201 mg, about 202 mg, about 203 mg, about 204 mg, about 205 mg, about 206 mg, about 207 mg, about 208 mg, about 209 mg, about 210 mg, about 211 mg, about 212 mg, about 213 mg, about 214 mg, about 215 mg, about 216 mg, about 217 mg, about 218 mg, about 219 mg, about 220 mg, about 221 mg, about 222 mg, about 223 mg, about 224 mg, about 225 mg, about 226 mg, about 227 mg, about 228 mg, about 229 mg, about 230 mg, about 231 mg, about 232 mg, about 233 mg, about 234 mg, about 235 mg, about 236 mg, about 237 mg, about 238 mg, about 239 mg, about 240 mg, about 241 mg, about 242 mg, about 243 mg, about 244 mg, about 245 mg, about 246 mg, about 247 mg, about 248 mg, about 249 mg, about 250 mg, about 251 mg, about 252 mg, about 253 mg, about 254 mg, about 255 mg, about 256 mg, about 257 mg, about 258 mg, about 259 mg, about 260 mg, about 261 mg, about 262 mg, about 263 mg, about 264 mg, about 265 mg, about 266 mg, about 267 mg, about 268 mg, about 269 mg, about 270 mg, about 271 mg, about 272 mg, about 273 mg, about 274 mg, about 275 mg, about 276 mg, about 277 mg, about 278 mg, about 279 mg, about 280 mg, about 281 mg, about 282 mg, about 283 mg, about 284 mg, about 285 mg, about 286 mg, about 287 mg, about 288 mg, about 289 mg, about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg, about 306 mg, about 307 mg, about 308 mg, about 309 mg, about 310 mg, about 311 mg, about 312 mg, about 313 mg, about 314 mg, about 315 mg, about 316 mg, about 317 mg, about 318 mg, about 319 mg, about 320 mg, about 321 mg, about 322 mg, about 323 mg, about 324 mg, about 325 mg, about 326 mg, about 327 mg, about 328 mg, about 329 mg, about 330 mg, about 331 mg, about 332 mg, about 333 mg, about 334 mg, about 335 mg, about 336 mg, about 337 mg, about 338 mg, about 339 mg, about 340 mg, about 341 mg, about 342 mg, about 343 mg, about 344 mg, about 345 mg, about 346 mg, about 347 mg, about 348 mg, about 349 mg, about 350 mg, about 351 mg, about 352 mg, about 353 mg, about 354 mg, about 355 mg, about 356 mg, about 357 mg, about 358 mg, about 359 mg, about 360 mg, about 361 mg, about 362 mg, about 363 mg, about 364 mg, about 365 mg, about 366 mg, about 367 mg, about 368 mg, about 369 mg, about 370 mg, about 371 mg, about 372 mg, about 373 mg, about 374 mg, about 375 mg, about 376 mg, about 377 mg, about 378 mg, about 379 mg, about 380 mg, about 381 mg, about 382 mg, about 383 mg, about 384 mg, about 385 mg, about 386 mg, about 387 mg, about 388 mg, about 389 mg, about 390 mg, about 391 mg, about 392 mg, about 393 mg, about 394 mg, about 395 mg, about 396 mg, about 397 mg, about 398 mg, about 399 mg, or 400 mg.

In some embodiments mg, the method as described herein comprises administering to the human subject a pharmaceutical composition comprising the compound as described herein at a first dose of 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, or 400 mg.

### Therapeutic Target Population

In some embodiments, the human subject is at most 18 years old. In some embodiments, the human subject is from 1 to 18, from 2 to 18, from 3 to 18, from 4 to 18, from 5 to 18, from 6 to 18, from 7 to 18, from 8 to 18, from 9 to 18, from 10 to 18, from 11 to 18, from 12 to 18, from 13 to 18, from 14 to 18, from 15 to 18, from 16 to 18, or from 17 to 18 years old. In some embodiments, the human subject is a human from 1 to 17, from 1 to 16, from 1 to 15, from 1 to 14, from 1 to 13, from 1 to 12, from 1 to 11, from 1 to 10, from 1 to 9, from 1 to 8, from 1 to 7, from 1 to 6, from 1 to 5, from 1 to 4, from 1 to 3, or from 1 to 2 years old. In some embodiments, the human subject is less than a year old or 1, 2, 3, 4, 5, 6, 7, 8, 9, 10, 11, 12, 13, 14, 15, 16, 17, or 18 years old.

In some embodiments, the human subject is at most 35 years, 30 years, 29 years, 28 years, 27 years, 26 years, 25 years, 24 years, 23 years, 22 years, 21 years, 20 years, 19 years, 18 years, 17 years, 16 years, 15 years, 14 years, 13 years, 12 years, 11 years, 10 years, 9 years, 8 years, 7 years, 6 years, 5 years, 4 years, 3 years, 2 years, or 1 year old.

In some embodiments, the human subject is less than a year old or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 years old.

In some embodiments, the human subject is from 1 to 35, from 2 to 35, from 3 to 35, from 4 to 35, from 5 to 35, from 6 to 35, from 7 to 35, from 8 to 35, from 9 to 35, from 10 to 35, from 11 to 35, from 12 to 35, from 13 to 35, from 14 to 35, from 15 to 35, from 16 to 35, 17 to 35, from 18 to 35, from 19 to 35, from 20 to 35, from 21 to 35, from 22 to 35, from 23 to 35, from 24 to 35, from 25 to 35, from 26 to 35, from 27 to 35, from 28 to 35, from 29 to 35, from 30 to 35, from 31 to 35, from 32 to 35, from 33 to 35, or from 34 to 35 years old.

In some embodiments, the human subject is a human from 1 to 35, from 1 to 34, from 1 to 33, from 1 to 32, from 1 to 31, from 1 to 30, from 1 to 29, from 1 to 28, from 1 to 27, from 1 to 26, from 1 to 25, from 1 to 24, from 1 to 23, from 1 to 22, from 1 to 21, from 1 to 20, from 1 to 19, from 1 to 18, from 1 to 17, from 1 to 16, from 1 to 15, from 1 to 14, from 1 to 13, from 1 to 12, from 1 to 11, from 1 to 10, from 1 to 9, from 1 to 8, from 1 to 7, from 1 to 6, from 1 to 5, from 1 to 4, from 1 to 3, or from 1 to 2 years old.

In some embodiments, the human subject is a human from 2 to 35, from 2 to 34, from 2 to 33, from 2 to 32, from 2 to 31, from 2 to 30, from 2 to 29, from 2 to 28, from 2 to 27, from 2 to 26, from 2 to 25, from 2 to 24, from 2 to 23, from 2 to 22, from 2 to 21, from 2 to 20, from 2 to 19, from 2 to 18, from 2 to 17, from 2 to 16, from 2 to 15, from 2 to 14, from 2 to 13, from 2 to 12, from 2 to 11, from 2 to 10, from 2 to 9, from 2 to 8, from 2 to 7, from 2 to 6, from 2 to 5, from 2 to 4, or from 2 to 3 years old. In some embodiments, the human subject is a human from 3 to 35, from 3 to 34, from 3 to 33, from 3 to 32, from 3 to 31, from 3 to 30, from 3 to 29, from 3 to 28, from 3 to 27, from 3 to 26, from 3 to 25, from 3 to 24, from 3 to 23, from 3 to 22, from 3 to 21, from 3 to 20, from 3 to 19, from 3 to 18, from 3 to 17, from 3 to 16, from 3 to 15, from 3 to 14, from 3 to 13, from 3 to 12, from 3 to 11, from 3 to 10, from 3 to 9, from 3 to 8, from 3 to 7, from 3 to 6, from 3 to 5, or from 3 to 4 years old.

In some embodiments, the human subject is a human from 4 to 35, from 4 to 34, from 4 to 33, from 4 to 32, from 4 to 31, from 4 to 30, from 4 to 29, from 4 to 28, from 4 to 27, from 4 to 26, from 4 to 25, from 4 to 24, from 4 to 23, from 4 to 22, from 4 to 21, from 4 to 20, from 4 to 19, from 4 to 18, from 4 to 17, from 4 to 16, from 4 to 15, from 4 to 14, from 4 to 13, from 4 to 12, from 4 to 11, from 4 to 10, from 4 to 9, from 4 to 8, from 4 to 7, from 4 to 6, or from 4 to 5 years old.

In some embodiments, the human subject is a human from 5 to 35, from 5 to 34, from 5 to 33, from 5 to 32, from 5 to 31, from 5 to 30, from 5 to 29, from 5 to 28, from 5 to 27, from 5 to 26, from 5 to 25, from 5 to 24, from 5 to 23, from 5 to 22, from 5 to 21, from 5 to 20, from 5 to 19, from 5 to 18, from 5 to 17, from 5 to 16, from 5 to 15, from 5 to 14, from 5 to 13, from 5 to 12, from 5 to 11, from 5 to 10, from 5 to 9, from 5 to 8, from 5 to 7, or from 5 to 6 years old.

In some embodiments, the human subject is a human from 6 to 35, from 6 to 34, from 6 to 33, from 6 to 32, from 6 to 31, from 6 to 30, from 6 to 29, from 6 to 28, from 6 to 27, from 6 to 26, from 6 to 25, from 6 to 24, from 6 to 23, from 6 to 22, from 6 to 21, from 6 to 20, from 6 to 19, from 6 to 18, from 6 to 17, from 6 to 16, from 6 to 15, from 6 to 14, from 6 to 13, from 6 to 12, from 6 to 11, from 6 to 10, from 6 to 9, from 6 to 8, or from 6 to 7 years old.

In some embodiments, the human subject is a human from 7 to 35, from 7 to 34, from 7 to 33, from 7 to 32, from 7 to 31, from 7 to 30, from 7 to 29, from 7 to 28, from 7 to 27, from 7 to 26, from 7 to 25, from 7 to 24, from 7 to 23, from 7 to 22, from 7 to 21, from 7 to 20, from 7 to 19, from 7 to 18, from 7 to 17, from 7 to 16, from 7 to 15, from 7 to 14, from 7 to 13, from 7 to 12, from 7 to 11, from 7 to 10, from 7 to 9, from 7 to 8 years old.

In some embodiments, the human subject is a human from 8 to 35, from 8 to 34, from 8 to 33, from 8 to 32, from 8 to 31, from 8 to 30, from 8 to 29, from 8 to 28, from 8 to 27, from 8 to 26, from 8 to 25, from 8 to 24, from 8 to 23, from 8 to 22, from 8 to 21, from 8 to 20, from 8 to 19, from 8 to 18, from 8 to 17, from 8 to 16, from 8 to 15, from 8 to 14, from 8 to 13, from 8 to 12, from 8 to 11, from 8 to 10, or from 8 to 9 years old.

In some embodiments, the human subject is characterized by having: (i) seizure onset prior to 12 months of age with recurrent focal motor or hemiconvulsive or generalized tonic-clonic seizures, which are often prolonged and triggered by hyperthermia; (ii) no past history of causal magnetic resonance imaging lesion; (iii) no other known etiology of any diseases or conditions except Dravet Syndrome; (iv) normal development at seizure onset; (v) a pathogenic variant, or variant of uncertain significance in an SCN1A gene; (vi) at least 2 prior treatments for epilepsy that either had lack of adequate seizure control; (vii) 4 or more convulsive seizures during the 28 days prior to administering, wherein the convulsive seizures is any one selected from Hemiclonic, Focal with Motor Signs, Focal to Bilateral Tonic Clonic Convulsion, Generalized Tonic Clonic Convulsion, Tonic, Tonic or Atonic (Drop Attacks), and Clonic; (viii) a current intervention for epilepsy or medication with at least one antiepileptic drug at a dose which has been stable for at least 4 weeks, wherein the interventions for epilepsy is a ketogenic diet, a vagal nerve stimulator, or a cannabinoid or marijuana-derived product; or (ix) any combination of (i)-(viii).

In some embodiments, the human subject is characterized by having at least one or more of: (i) seizure onset prior to 12 months of age with recurrent focal motor or hemiconvulsive or generalized tonic-clonic seizures, which are often prolonged and triggered by hyperthermia; (ii) no past history of causal magnetic resonance imaging lesion; (iii) no other known etiology of any diseases or conditions except Dravet Syndrome; (iv) normal development at seizure onset; (v) a pathogenic variant, or variant of uncertain significance in an SCN1A gene; (vi) at least 2 prior treatments for epilepsy that either had lack of adequate seizure control; (vii) 4 or more convulsive seizures during the 28 days prior to administering, wherein the convulsive seizures is any one selected from Hemiclonic, Focal with Motor Signs, Focal to Bilateral Tonic Clonic Convulsion, Generalized Tonic Clonic Convulsion, Tonic, Tonic or Atonic (Drop Attacks), and Clonic; and (viii) a current intervention for epilepsy or medication with at least one antiepileptic drug at a dose which has been stable for at least 4 weeks, wherein the interventions for epilepsy is a ketogenic diet, a vagal nerve stimulator, or a cannabinoid or marijuana-derived product. In some embodiments, the human subject is characterized by having at least two or more of: (i) seizure onset prior to 12 months of age with recurrent focal motor or hemiconvulsive or generalized tonic-clonic seizures, which are often prolonged and triggered by hyperthermia; (ii) no past history of causal magnetic resonance imaging lesion; (iii) no other known etiology of any diseases or conditions except Dravet Syndrome; (iv) normal development at seizure onset; (v) a pathogenic variant, or variant of uncertain significance in an SCN1A gene; (vi) at least 2 prior treatments for epilepsy that either had lack of adequate seizure control; (vii) 4 or more convulsive seizures during the 28 days prior to administering, wherein the convulsive seizures is any one selected from Hemiclonic, Focal with Motor Signs, Focal to Bilateral Tonic Clonic Convulsion, Generalized Tonic Clonic Convulsion, Tonic, Tonic or Atonic (Drop Attacks), and Clonic; and (viii) a current intervention for epilepsy or medication with at least one antiepileptic drug at a dose which has been stable for at least 4 weeks, wherein the interventions for epilepsy is a ketogenic diet, a vagal nerve stimulator, or a cannabinoid or marijuana-derived product. In some embodiments, the human subject is characterized by having at least three or more of: (i) seizure onset prior to 12 months of age with recurrent focal motor or hemiconvulsive or generalized tonic-clonic seizures, which are often prolonged and triggered by hyperthermia; (ii) no past history of causal magnetic resonance imaging lesion; (iii) no other known etiology of any diseases or conditions except Dravet Syndrome; (iv) normal development at seizure onset; (v) a pathogenic variant, or variant of uncertain significance in an SCN1A gene; (vi) at least 2 prior treatments for epilepsy that either had lack of adequate seizure control; (vii) 4 or more convulsive seizures during the 28 days prior to administering, wherein the convulsive seizures is any one selected from Hemiclonic, Focal with Motor Signs, Focal to Bilateral Tonic Clonic Convulsion, Generalized Tonic Clonic Convulsion, Tonic, Tonic or Atonic (Drop Attacks), and Clonic; and (viii) a current intervention for epilepsy or medication with at least one antiepileptic drug at a dose which has been stable for at least 4 weeks, wherein the interventions for epilepsy is a ketogenic diet, a vagal nerve stimulator, or a cannabinoid or marijuana-derived product. In some embodiments, the human subject is characterized by having at least four or more of: (i) seizure onset prior to 12 months of age with recurrent focal motor or hemiconvulsive or generalized tonic-clonic seizures, which are often prolonged and triggered by hyperthermia; (ii) no past history of causal magnetic resonance imaging lesion; (iii) no other known etiology of any diseases or conditions except Dravet Syndrome; (iv) normal development at seizure onset; (v) a pathogenic variant, or variant of uncertain significance in an SCN1A gene; (vi) at least 2 prior treatments for epilepsy that either had lack of adequate seizure control; (vii) 4 or more convulsive seizures during the 28 days prior to administering, wherein the convulsive seizures is any one selected from Hemiclonic, Focal with Motor Signs, Focal to Bilateral Tonic Clonic Convulsion, Generalized Tonic Clonic Convulsion, Tonic, Tonic or Atonic (Drop Attacks), and Clonic; and (viii) a current intervention for epilepsy or medication with at least one antiepileptic drug at a dose which has been stable for at least 4 weeks, wherein the interventions for epilepsy is a ketogenic diet, a vagal nerve stimulator, or a cannabinoid or marijuana-derived product. In some embodiments, the human subject is characterized by having at least five or more of: (i) seizure onset prior to 12 months of age with recurrent focal motor or hemiconvulsive or generalized tonic-clonic seizures, which are often prolonged and triggered by hyperthermia; (ii) no past history of causal magnetic resonance imaging lesion; (iii) no other known etiology of any diseases or conditions except Dravet Syndrome; (iv) normal development at seizure onset; (v) a pathogenic variant, or variant of uncertain significance in an SCN1A gene; (vi) at least 2 prior treatments for epilepsy that either had lack of adequate seizure control; (vii) 4 or more convulsive seizures during the 28 days prior to administering, wherein the convulsive seizures is any one selected from Hemiclonic, Focal with Motor Signs, Focal to Bilateral Tonic Clonic Convulsion, Generalized Tonic Clonic Convulsion, Tonic, Tonic or Atonic (Drop Attacks), and Clonic; and (viii) a current intervention for epilepsy or medication with at least one antiepileptic drug at a dose which has been stable for at least 4 weeks, wherein the interventions for epilepsy is a ketogenic diet, a vagal nerve stimulator, or a cannabinoid or marijuana-derived product. In some embodiments, the human subject is characterized by having at least six or more of: (i) seizure onset prior to 12 months of age with recurrent focal motor or hemiconvulsive or generalized tonic-clonic seizures, which are often prolonged and triggered by hyperthermia; (ii) no past history of causal magnetic resonance imaging lesion; (iii) no other known etiology of any diseases or conditions except Dravet Syndrome; (iv) normal development at seizure onset; (v) a pathogenic variant, or variant of uncertain significance in an SCN1A gene; (vi) at least 2 prior treatments for epilepsy that either had lack of adequate seizure control; (vii) 4 or more convulsive seizures during the 28 days prior to administering, wherein the convulsive seizures is any one selected from Hemiclonic, Focal with Motor Signs, Focal to Bilateral Tonic Clonic Convulsion, Generalized Tonic Clonic Convulsion, Tonic, Tonic or Atonic (Drop Attacks), and Clonic; and (viii) a current intervention for epilepsy or medication with at least one antiepileptic drug at a dose which has been stable for at least 4 weeks, wherein the interventions for epilepsy is a ketogenic diet, a vagal nerve stimulator, or a cannabinoid or marijuana-derived product. In some embodiments, the human subject is characterized by having at least seven or more of: (i) seizure onset prior to 12 months of age with recurrent focal motor or hemiconvulsive or generalized tonic-clonic seizures, which are often prolonged and triggered by hyperthermia; (ii) no past history of causal magnetic resonance imaging lesion; (iii) no other known etiology of any diseases or conditions except Dravet Syndrome; (iv) normal development at seizure onset; (v) a pathogenic variant, or variant of uncertain significance in an SCN1A gene; (vi) at least 2 prior treatments for epilepsy that either had lack of adequate seizure control; (vii) 4 or more convulsive seizures during the 28 days prior to administering, wherein the convulsive seizures is any one selected from Hemiclonic, Focal with Motor Signs, Focal to Bilateral Tonic Clonic Convulsion, Generalized Tonic Clonic Convulsion, Tonic, Tonic or Atonic (Drop Attacks), and Clonic; and (viii) a current intervention for epilepsy or medication with at least one antiepileptic drug at a dose which has been stable for at least 4 weeks, wherein the interventions for epilepsy is a ketogenic diet, a vagal nerve stimulator, or a cannabinoid or marijuana-derived product. In some embodiments, the human subject is characterized by having all eight of: (i) seizure onset prior to 12 months of age with recurrent focal motor or hemiconvulsive or generalized tonic-clonic seizures, which are often prolonged and triggered by hyperthermia; (ii) no past history of causal magnetic resonance imaging lesion; (iii) no other known etiology of any diseases or conditions except Dravet Syndrome; (iv) normal development at seizure onset; (v) a pathogenic variant, or variant of uncertain significance in an SCN1A gene; (vi) at least 2 prior treatments for epilepsy that either had lack of adequate seizure control; (vii) 4 or more convulsive seizures during the 28 days prior to administering, wherein the convulsive seizures is any one selected from Hemiclonic, Focal with Motor Signs, Focal to Bilateral Tonic Clonic Convulsion, Generalized Tonic Clonic Convulsion, Tonic, Tonic or Atonic (Drop Attacks), and Clonic; and (viii) a current intervention for epilepsy or medication with at least one antiepileptic drug at a dose which has been stable for at least 4 weeks, wherein the interventions for epilepsy is a ketogenic diet, a vagal nerve stimulator, or a cannabinoid or marijuana-derived product.

In some embodiments, the human subject is additionally characterized by not having one or more of the following: (a) one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr; (b) a known pathogenic mutation in another gene that causes epilepsy, wherein the pathogenic mutation is homozygous in cases of known recessive disease; (c) currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; (d) clinically, significantly unstable medical conditions other than epilepsy; (e) clinically, relevant symptoms or a clinically significant illness in the 4 weeks prior to administering, other than epilepsy; (f) a history of brain or spinal cord disease other than epilepsy or Dravet Syndrome; or a history of bacterial meningitis or brain malformation; (g) a spinal deformity or other condition that alters the free flow of cerebrospinal fluid (CSF) or having an implanted CSF drainage shunt; (h) clinically significant abnormal laboratory values prior to administering; (i) aspartate aminotransferase or alanine aminotransferase >2.5-fold upper limit of normal, serum creatinine >upper limit of normal or platelet count <lower limit of normal; (j) clinically relevant abnormalities in the 12-lead electrocardiogram (ECG) measured at prior to administering; (k) a psychiatric or behavioral disorder; (l) currently or in the past 4 weeks, medication of an anticoagulant, wherein the anticoagulant is not aspirin; or (m) any combination of (a)-(l). In some embodiments, the human subject is characterized by not having one or more of the following: (a) one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr; (b) a known pathogenic mutation in another gene that causes epilepsy, wherein the pathogenic mutation is homozygous in cases of known recessive disease; (c) currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; (d) clinically, significantly unstable medical conditions other than epilepsy; (e) clinically, relevant symptoms or a clinically significant illness in the 4 weeks prior to administering, other than epilepsy; (f) a history of brain or spinal cord disease other than epilepsy or Dravet Syndrome; or a history of bacterial meningitis or brain malformation; (g) a spinal deformity or other condition that alters the free flow of cerebrospinal fluid (CSF) or having an implanted CSF drainage shunt; (h) clinically significant abnormal laboratory values prior to administering; (i) aspartate aminotransferase or alanine aminotransferase >2.5-fold upper limit of normal, serum creatinine >upper limit of normal or platelet count <lower limit of normal; (j) clinically relevant abnormalities in the 12-lead electrocardiogram (ECG) measured at prior to administering; (k) a psychiatric or behavioral disorder; and (l) currently or in the past 4 weeks, medication of an anticoagulant, wherein the anticoagulant is not aspirin. In some embodiments, the human subject is additionally characterized by not having two or more of the following: (a) one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr; (b) a known pathogenic mutation in another gene that causes epilepsy, wherein the pathogenic mutation is homozygous in cases of known recessive disease; (c) currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; (d) clinically, significantly unstable medical conditions other than epilepsy; (e) clinically, relevant symptoms or a clinically significant illness in the 4 weeks prior to administering, other than epilepsy; (f) a history of brain or spinal cord disease other than epilepsy or Dravet Syndrome; or a history of bacterial meningitis or brain malformation; (g) a spinal deformity or other condition that alters the free flow of cerebrospinal fluid (CSF) or having an implanted CSF drainage shunt; (h) clinically significant abnormal laboratory values prior to administering; (i) aspartate aminotransferase or alanine aminotransferase >2.5-fold upper limit of normal, serum creatinine >upper limit of normal or platelet count <lower limit of normal; (j) clinically relevant abnormalities in the 12-lead electrocardiogram (ECG) measured at prior to administering; (k) a psychiatric or behavioral disorder; and (l) currently or in the past 4 weeks, medication of an anticoagulant, wherein the anticoagulant is not aspirin. In some embodiments, the human subject is additionally characterized by not having three or more of the following: (a) one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr; (b) a known pathogenic mutation in another gene that causes epilepsy, wherein the pathogenic mutation is homozygous in cases of known recessive disease; (c) currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; (d) clinically, significantly unstable medical conditions other than epilepsy; (e) clinically, relevant symptoms or a clinically significant illness in the 4 weeks prior to administering, other than epilepsy; (f) a history of brain or spinal cord disease other than epilepsy or Dravet Syndrome; or a history of bacterial meningitis or brain malformation; (g) a spinal deformity or other condition that alters the free flow of cerebrospinal fluid (CSF) or having an implanted CSF drainage shunt; (h) clinically significant abnormal laboratory values prior to administering; (i) aspartate aminotransferase or alanine aminotransferase >2.5-fold upper limit of normal, serum creatinine >upper limit of normal or platelet count <lower limit of normal; (j) clinically relevant abnormalities in the 12-lead electrocardiogram (ECG) measured at prior to administering; (k) a psychiatric or behavioral disorder; and (l) currently or in the past 4 weeks, medication of an anticoagulant, wherein the anticoagulant is not aspirin. In some embodiments, the human subject is additionally characterized by not having four or more of the following: (a) one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr; (b) a known pathogenic mutation in another gene that causes epilepsy, wherein the pathogenic mutation is homozygous in cases of known recessive disease; (c) currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; (d) clinically, significantly unstable medical conditions other than epilepsy; (e) clinically, relevant symptoms or a clinically significant illness in the 4 weeks prior to administering, other than epilepsy; (f) a history of brain or spinal cord disease other than epilepsy or Dravet Syndrome; or a history of bacterial meningitis or brain malformation; (g) a spinal deformity or other condition that alters the free flow of cerebrospinal fluid (CSF) or having an implanted CSF drainage shunt; (h) clinically significant abnormal laboratory values prior to administering; (i) aspartate aminotransferase or alanine aminotransferase >2.5-fold upper limit of normal, serum creatinine >upper limit of normal or platelet count <lower limit of normal; (j) clinically relevant abnormalities in the 12-lead electrocardiogram (ECG) measured at prior to administering; (k) a psychiatric or behavioral disorder; and (l) currently or in the past 4 weeks, medication of an anticoagulant, wherein the anticoagulant is not aspirin. In some embodiments, the human subject is additionally characterized by not having five or more of the following: (a) one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr; (b) a known pathogenic mutation in another gene that causes epilepsy, wherein the pathogenic mutation is homozygous in cases of known recessive disease; (c) currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; (d) clinically, significantly unstable medical conditions other than epilepsy; (e) clinically, relevant symptoms or a clinically significant illness in the 4 weeks prior to administering, other than epilepsy; (f) a history of brain or spinal cord disease other than epilepsy or Dravet Syndrome; or a history of bacterial meningitis or brain malformation; (g) a spinal deformity or other condition that alters the free flow of cerebrospinal fluid (CSF) or having an implanted CSF drainage shunt; (h) clinically significant abnormal laboratory values prior to administering; (i) aspartate aminotransferase or alanine aminotransferase >2.5-fold upper limit of normal, serum creatinine >upper limit of normal or platelet count <lower limit of normal; (j) clinically relevant abnormalities in the 12-lead electrocardiogram (ECG) measured at prior to administering; (k) a psychiatric or behavioral disorder; and (l) currently or in the past 4 weeks, medication of an anticoagulant, wherein the anticoagulant is not aspirin. In some embodiments, the human subject is additionally characterized by not having six or more of the following: (a) one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr; (b) a known pathogenic mutation in another gene that causes epilepsy, wherein the pathogenic mutation is homozygous in cases of known recessive disease; (c) currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; (d) clinically, significantly unstable medical conditions other than epilepsy; (e) clinically, relevant symptoms or a clinically significant illness in the 4 weeks prior to administering, other than epilepsy; (f) a history of brain or spinal cord disease other than epilepsy or Dravet Syndrome; or a history of bacterial meningitis or brain malformation; (g) a spinal deformity or other condition that alters the free flow of cerebrospinal fluid (CSF) or having an implanted CSF drainage shunt; (h) clinically significant abnormal laboratory values prior to administering; (i) aspartate aminotransferase or alanine aminotransferase >2.5-fold upper limit of normal, serum creatinine >upper limit of normal or platelet count <lower limit of normal; (j) clinically relevant abnormalities in the 12-lead electrocardiogram (ECG) measured at prior to administering; (k) a psychiatric or behavioral disorder; and (l) currently or in the past 4 weeks, medication of an anticoagulant, wherein the anticoagulant is not aspirin. In some embodiments, the human subject is additionally characterized by not having seven or more of the following: (a) one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr; (b) a known pathogenic mutation in another gene that causes epilepsy, wherein the pathogenic mutation is homozygous in cases of known recessive disease; (c) currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; (d) clinically, significantly unstable medical conditions other than epilepsy; (e) clinically, relevant symptoms or a clinically significant illness in the 4 weeks prior to administering, other than epilepsy; (f) a history of brain or spinal cord disease other than epilepsy or Dravet Syndrome; or a history of bacterial meningitis or brain malformation; (g) a spinal deformity or other condition that alters the free flow of cerebrospinal fluid (CSF) or having an implanted CSF drainage shunt; (h) clinically significant abnormal laboratory values prior to administering; (i) aspartate aminotransferase or alanine aminotransferase >2.5-fold upper limit of normal, serum creatinine >upper limit of normal or platelet count <lower limit of normal; (j) clinically relevant abnormalities in the 12-lead electrocardiogram (ECG) measured at prior to administering; (k) a psychiatric or behavioral disorder; and (l) currently or in the past 4 weeks, medication of an anticoagulant, wherein the anticoagulant is not aspirin. In some embodiments, the human subject is additionally characterized by not having eight or more of the following: (a) one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr; (b) a known pathogenic mutation in another gene that causes epilepsy, wherein the pathogenic mutation is homozygous in cases of known recessive disease; (c) currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; (d) clinically, significantly unstable medical conditions other than epilepsy; (e) clinically, relevant symptoms or a clinically significant illness in the 4 weeks prior to administering, other than epilepsy; (f) a history of brain or spinal cord disease other than epilepsy or Dravet Syndrome; or a history of bacterial meningitis or brain malformation; (g) a spinal deformity or other condition that alters the free flow of cerebrospinal fluid (CSF) or having an implanted CSF drainage shunt; (h) clinically significant abnormal laboratory values prior to administering; (i) aspartate aminotransferase or alanine aminotransferase >2.5-fold upper limit of normal, serum creatinine >upper limit of normal or platelet count <lower limit of normal; (j) clinically relevant abnormalities in the 12-lead electrocardiogram (ECG) measured at prior to administering; (k) a psychiatric or behavioral disorder; and (l) currently or in the past 4 weeks, medication of an anticoagulant, wherein the anticoagulant is not aspirin. In some embodiments, the human subject is additionally characterized by not having nine or more of the following: (a) one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr; (b) a known pathogenic mutation in another gene that causes epilepsy, wherein the pathogenic mutation is homozygous in cases of known recessive disease; (c) currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; (d) clinically, significantly unstable medical conditions other than epilepsy; (e) clinically, relevant symptoms or a clinically significant illness in the 4 weeks prior to administering, other than epilepsy; (f) a history of brain or spinal cord disease other than epilepsy or Dravet Syndrome; or a history of bacterial meningitis or brain malformation; (g) a spinal deformity or other condition that alters the free flow of cerebrospinal fluid (CSF) or having an implanted CSF drainage shunt; (h) clinically significant abnormal laboratory values prior to administering; (i) aspartate aminotransferase or alanine aminotransferase >2.5-fold upper limit of normal, serum creatinine >upper limit of normal or platelet count <lower limit of normal; (j) clinically relevant abnormalities in the 12-lead electrocardiogram (ECG) measured at prior to administering; (k) a psychiatric or behavioral disorder; and (l) currently or in the past 4 weeks, medication of an anticoagulant, wherein the anticoagulant is not aspirin. In some embodiments, the human subject is additionally characterized by not having ten or more of the following: (a) one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr; (b) a known pathogenic mutation in another gene that causes epilepsy, wherein the pathogenic mutation is homozygous in cases of known recessive disease; (c) currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; (d) clinically, significantly unstable medical conditions other than epilepsy; (e) clinically, relevant symptoms or a clinically significant illness in the 4 weeks prior to administering, other than epilepsy; (f) a history of brain or spinal cord disease other than epilepsy or Dravet Syndrome; or a history of bacterial meningitis or brain malformation; (g) a spinal deformity or other condition that alters the free flow of cerebrospinal fluid (CSF) or having an implanted CSF drainage shunt; (h) clinically significant abnormal laboratory values prior to administering; (i) aspartate aminotransferase or alanine aminotransferase >2.5-fold upper limit of normal, serum creatinine >upper limit of normal or platelet count <lower limit of normal; (j) clinically relevant abnormalities in the 12-lead electrocardiogram (ECG) measured at prior to administering; (k) a psychiatric or behavioral disorder; and (l) currently or in the past 4 weeks, medication of an anticoagulant, wherein the anticoagulant is not aspirin. In some embodiments, the human subject is additionally characterized by not having eleven or more of the following: (a) one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr; (b) a known pathogenic mutation in another gene that causes epilepsy, wherein the pathogenic mutation is homozygous in cases of known recessive disease; (c) currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; (d) clinically, significantly unstable medical conditions other than epilepsy; (e) clinically, relevant symptoms or a clinically significant illness in the 4 weeks prior to administering, other than epilepsy; (f) a history of brain or spinal cord disease other than epilepsy or Dravet Syndrome; or a history of bacterial meningitis or brain malformation; (g) a spinal deformity or other condition that alters the free flow of cerebrospinal fluid (CSF) or having an implanted CSF drainage shunt; (h) clinically significant abnormal laboratory values prior to administering; (i) aspartate aminotransferase or alanine aminotransferase >2.5-fold upper limit of normal, serum creatinine >upper limit of normal or platelet count <lower limit of normal; (j) clinically relevant abnormalities in the 12-lead electrocardiogram (ECG) measured at prior to administering; (k) a psychiatric or behavioral disorder; and (l) currently or in the past 4 weeks, medication of an anticoagulant, wherein the anticoagulant is not aspirin. In some embodiments, the human subject is additionally characterized by not having all of the following: (a) one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, and Asp1866Tyr; (b) a known pathogenic mutation in another gene that causes epilepsy, wherein the pathogenic mutation is homozygous in cases of known recessive disease; (c) currently treated with a sodium channel blocker as maintenance treatment and an anticoagulant, wherein the sodium channel blocker is phenytoin, carbamazepine, oxcarbazepine, lamotrigine, lacosamide, or rufinamide, and wherein the anticoagulant is not an aspirin; (d) clinically, significantly unstable medical conditions other than epilepsy; (e) clinically, relevant symptoms or a clinically significant illness in the 4 weeks prior to administering, other than epilepsy; (f) a history of brain or spinal cord disease other than epilepsy or Dravet Syndrome; or a history of bacterial meningitis or brain malformation; (g) a spinal deformity or other condition that alters the free flow of cerebrospinal fluid (CSF) or having an implanted CSF drainage shunt; (h) clinically significant abnormal laboratory values prior to administering; (i) aspartate aminotransferase or alanine aminotransferase >2.5-fold upper limit of normal, serum creatinine >upper limit of normal or platelet count <lower limit of normal; (j) clinically relevant abnormalities in the 12-lead electrocardiogram (ECG) measured at prior to administering; (k) a psychiatric or behavioral disorder; and (l) currently or in the past 4 weeks, medication of an anticoagulant, wherein the anticoagulant is not aspirin.

In some embodiments, the human subject is additionally characterized by not having known pathogenic mutation in another gene that causes epilepsy. In some embodiments, the human subject is additionally characterized by not having had clinically relevant symptoms or a clinically significant illness in the past 4 weeks other than epilepsy. In some embodiments, the human subject is additionally characterized by not having specific mutations of SCN1A gene demonstrated to cause gain-of-function. In some embodiments, the human subject is additionally characterized by currently not being treated with an anti-epileptic drug acting predominantly as a sodium channel blocker. In some embodiments, the human subject is additionally characterized by not having clinically significant unstable medical condition(s) other than epilepsy.

In some embodiments, the human subject has pediatric epilepsy, epileptic encephalopathies, refractory myoclonic epilepsy, or severe myoclonic epilepsy in infancy. In some embodiments, the human subject has myoclonic epilepsies, generalized epilepsy, epilepsy, brain diseases, central nervous system diseases, nervous system diseases, or epileptic syndromes. In some embodiments, the methods of treatment as described herein comprise methods of treating or reducing the likelihood of developing a disease or condition, wherein the disease or condition is pediatric epilepsy, epileptic encephalopathies, refractory myoclonic epilepsy, or severe myoclonic epilepsy in infancy. In some embodiments, the methods of treatment as described herein comprise methods of treating or reducing the likelihood of developing a disease or condition, wherein the disease or condition is myoclonic epilepsies, generalized epilepsy, epilepsy, brain diseases, central nervous system diseases, nervous system diseases, or epileptic syndromes. In some embodiments, the human subject has seizures that are not controlled by current antiepileptic drug (AED) regimen. In some embodiments, the AED regimen comprises clobazam, cannabidiol, levetiracetam, stiripentol, or valproic acid/valproate.

The term “magnetic resonance imaging lesion,” as used herein, refers to any damage or abnormal change in the tissue of an organism, caused by the magnetic resonance imaging. The term “magnetic resonance imaging,” as used herein, refers to a form of medical imaging that measures the response of the atomic nuclei of body tissues to high-frequency radio waves when placed in a strong magnetic field, and that produces images of the internal organs.

The term “ketogenic diet,” as used herein, refers to a high-fat, adequate-protein, low-carbohydrate diet that in medicine is used, for example, to treat refractory epilepsy in children. The diet forces the body to burn fats rather than carbohydrates.

The term “a vagal nerve stimulator” or “vagus nerve stimulation (VNS)” as used herein, refers to a medical treatment that involves delivering electrical impulses to the vagus nerve. It is, for example, used as an add-on treatment for certain types of intractable epilepsy and treatment-resistant depression.

The term “cannabinoid,” as used herein, refers to a chemical found in cannabis. Exemplary cannabinoids include, but are not limited to, the phytocannabinoid tetrahydrocannabinol (THC) (Delta9-THC or Delta8-THC), and cannabidiol (CBD). Cannabinoids, as used herein, may be natural or synthetic chemicals.

The term “marijuana” or “cannabis,” as used herein, refers to a psychoactive drug from the Cannabis plant used primarily for medical or recreational purposes. An exemplary main psychoactive component of cannabis is tetrahydrocannabinol (THC).

The term “sodium channel blocker,” as used herein, refers to a drug which impair the conduction of sodium ions (Na+) through sodium channels. Examples of sodium channel blockers include, but are not limited to, alkaloids (e.g., saxitoxin, neosaxitoxin, tetrodotoxin), local anesthetics (e.g., lidocaine), anticonvulsants (e.g., phenytoin, oxcarbazepine (derivative of carbamazepine)), and class Ia (e.g., quinidine, procainamide and disopyramide), class Ib (e.g., lidocaine, mexiletine, tocainide, and phenytoin) and class Ic (e.g., encainide, flecainide, moricizine, and propafenone) antiarrhythmic agents.

The term “cerebrospinal fluid (CSF),” as used herein, refers to a clear, colorless body fluid found in the brain and spinal cord. CSF, for example, acts as a cushion or buffer, providing basic mechanical and immunological protection to the brain inside the skull, and plays a vital function in the cerebral autoregulation of cerebral blood flow. The term “artificial cerebrospinal fluid (aCSF),” as used herein, refers to a biological buffer solution that is commonly used as a vehicle solution for administration of agents to the central nervous system (CNS). aCSF, for instance, closely matches the electrolyte concentrations and physiological compatibility of endogenous CSF to enable a vital environment for neuronal tissue by maintaining the homeostasis, osmolarity, and pH at physiological levels.

The term “CSF drainage shunt,” as used herein, refers to a system that drains excess fluid from the brain to another part of the body where the fluid is absorbed as part of the circulatory process. CSF shunts are, for example, used to treat hydrocephalus.

The term “electrocardiogram (EKG or ECG),” as used herein, refers to a test that measures the electrical activity of the heartbeat, e.g., producing a graph of voltage versus time of the electrical activity of the heart. With each beat, an electrical impulse (or “wave”) travels through the heart.

“Aspartate transaminase (AST),” also known as aspartate aminotransferase, AspAT/ASAT/AAT, or (serum) glutamic oxaloacetic transaminase (GOT, SGOT), as used herein, refers to a pyridoxal phosphate (PLP)-dependent transaminase enzyme (EC 2.6.1.1). AST includes any of the recombinant or naturally occurring forms of AST protein or variants or homologs thereof that maintain AST activity, (e.g., within at least 50%, 80%, 90%, 95%, 96%, 97%, 98%, 99% or 100% activity compared to AST). Exemplary AST activity includes, but are not limited to, playing a role in amino acid metabolism, for example, by catalyzing the reversible transfer of an α-amino group between aspartate and glutamate and, as such. In some aspects, the variants or homologs have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring AST protein. In some embodiments, the AST protein is substantially identical to the protein identified by the UniProt reference number P17174 or a variant or homolog having substantial identity thereto. In some embodiments, the AST protein is substantially identical to the protein identified by the UniProt reference number P00505 or a variant or homolog having substantial identity thereto.

“Alanine transaminase (ALT),” also known as alanine aminotransferase (ALAT), serum glutamate-pyruvate transaminase (SGPT), or serum glutamic-pyruvic transaminase (SGPT), as used herein, refers to a transaminase enzyme (EC 2.6.1.2). ALT includes any of the recombinant or naturally occurring forms of ALT protein or variants or homologs thereof that maintain ALT activity, (e.g., within at least 50%, 80%, 90%, 95%, 96%, 97%, 98%, 99% or 100% activity compared to ALT). Exemplary ALT activity includes, but are not limited to, catalyzing the two parts of the alanine cycle. In some aspects, the variants or homologs have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring ALT protein. In some embodiments, the ALT protein is substantially identical to the protein identified by the UniProt reference number P24298 or a variant or homolog having substantial identity thereto.

In some embodiments, serum AST level, serum ALT level, and their ratio (AST/ALT ratio) are measured clinically as biomarkers for liver health.

The term “laboratory vale,” as used herein refers to the value obtained by laboratory tests or measurements. Exemplary, non-limiting laboratory tests or measurements may be related to hematology, coagulation, clinical chemistry, plasma, urinalysis, serum, serum or urine pregnancy, urine, or cerebrospinal fluid.

### Therapeutic Schedule

In some embodiments, the first dose is a single dose. In some embodiments, the first dose is the first of multiple doses. In some embodiments, the method further comprises assessing tolerability or effectiveness of the pharmaceutical composition.

In some embodiments, the method as described herein further comprises administering to the human subject a pharmaceutical composition comprising the compound as described herein at a subsequent dose of 0.1, 0.5, 1, 2.5, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mg.

In some embodiments, the method as described herein further comprises administering to the human subject a pharmaceutical composition comprising the compound as described herein at a subsequent dose of from about 0.1 to about 1000 mg, from about 0.2 to about 1000 mg, from about 0.3 to about 1000 mg, from about 0.4 to about 1000 mg, from about 0.5 to about 1000 mg, from about 0.6 to about 1000 mg, from about 0.7 to about 1000 mg, from about 0.8 to about 1000 mg, from about 0.9 to about 1000 mg, 1 to about 1000 mg, from about 2 to about 1000 mg, from about 3 to about 1000 mg, from about 4 to about 1000 mg, from about 5 to about 1000 mg, from about 6 to about 1000 mg, from about 7 to about 1000 mg, from about 8 to about 1000 mg, from about 9 to about 1000 mg, from about 10 to about 1000 mg, from about 15 to about 1000 mg, from about 20 to about 1000 mg, from about 25 to about 1000 mg, from about 30 to about 1000 mg, from about 35 to about 1000 mg, from about 40 to about 1000 mg, from about 45 to about 1000 mg, from about 50 to about 1000 mg, from about 55 to about 1000 mg, from about 60 to about 1000 mg, from about 65 to about 1000 mg, from about 70 to about 1000 mg, from about 75 to about 1000 mg, from about 80 to about 1000 mg, from about 85 to about 1000 mg, from about 90 to about 1000 mg, from about 95 to about 1000 mg, from about 100 to about 1000 mg, from about 150 to about 1000 mg, from about 200 to about 1000 mg, from about 250 to about 1000 mg, from about 300 to about 1000 mg, from about 350 to about 1000 mg, from about 400 to about 1000 mg, from about 450 to about 1000 mg, from about 500 to about 1000 mg, from about 550 to about 1000 mg, from about 600 to about 1000 mg, from about 650 to about 1000 mg, from about 700 to about 1000 mg, from about 750 to about 1000 mg, from about 800 to about 1000 mg, from about 850 to about 1000 mg, from about 900 to about 1000 mg, or from about 950 to about 1000 mg.

In some embodiments, the method as described herein further comprises administering to the human subject a pharmaceutical composition comprising the compound as described herein at a subsequent dose of from 0.1 to 1000 mg, from 0.2 to 1000 mg, from 0.3 to 1000 mg, from 0.4 to 1000 mg, from 0.5 to 1000 mg, from 0.6 to 1000 mg, from 0.7 to 1000 mg, from 0.8 to 1000 mg, from 0.9 to 1000 mg, 1 to 1000 mg, from 2 to 1000 mg, from 3 to 1000 mg, from 4 to 1000 mg, from 5 to 1000 mg, from 6 to 1000 mg, from 7 to 1000 mg, from 8 to 1000 mg, from 9 to 1000 mg, from 10 to 1000 mg, from 15 to 1000 mg, from 20 to 1000 mg, from 25 to 1000 mg, from 30 to 1000 mg, from 35 to 1000 mg, from 40 to 1000 mg, from 45 to 1000 mg, from 50 to 1000 mg, from 55 to 1000 mg, from 60 to 1000 mg, from 65 to 1000 mg, from 70 to 1000 mg, from 75 to 1000 mg, from 80 to 1000 mg, from 85 to 1000 mg, from 90 to 1000 mg, from 95 to 1000 mg, from 100 to 1000 mg, from 150 to 1000 mg, from 200 to 1000 mg, from 250 to 1000 mg, from 300 to 1000 mg, from 350 to 1000 mg, from 400 to 1000 mg, from 450 to 1000 mg, from 500 to 1000 mg, from 550 to 1000 mg, from 600 to 1000 mg, from 650 to 1000 mg, from 700 to 1000 mg, from 750 to 1000 mg, from 800 to 1000 mg, from 850 to 1000 mg, from 900 to 1000 mg, or from 950 to 1000 mg.

In some embodiments, the method as described herein further comprises administering to the human subject a pharmaceutical composition comprising the compound as described herein at a subsequent dose of from about 0.1 to about 950 mg, from about 0.1 to about 900 mg, from about 0.1 to about 850 mg, from about 0.1 to about 800 mg, from about 0.1 to about 750 mg, from about 0.1 to about 700 mg, from about 0.1 to about 650 mg, from about 0.1 to about 600 mg, from about 0.1 to about 550 mg, from about 0.1 to about 500 mg, from about 0.1 to about 450 mg, from about 0.1 to about 400 mg, from about 0.1 to about 350 mg, from about 0.1 to about 300 mg, from about 0.1 to about 250 mg, from about 0.1 to about 200 mg, from about 0.1 to about 150 mg, from about 0.1 to about 100 mg, from about 0.1 to about 95 mg, from about 0.1 to about 90 mg, from about 0.1 to about 85 mg, from about 0.1 to about 80 mg, from about 0.1 to about 75 mg, from about 0.1 to about 70 mg, from about 0.1 to about 65 mg, from about 0.1 to about 60 mg, from about 0.1 to about 55 mg, from about 0.1 to about 50 mg, from about 0.1 to about 45 mg, from about 0.1 to about 40 mg, from about 0.1 to about 35 mg, from about 0.1 to about 30 mg, from about 0.1 to about mg, from about 0.1 to about 25 mg, from about 0.1 to about 20 mg, from about 0.1 to about 10 mg, from about 0.1 to about 9 mg, from about 0.1 to about 8 mg, from about 0.1 to about 7 mg, from about 0.1 to about 6 mg, from about 0.1 to about 5 mg, from about 0.1 to about 4 mg, from about 0.1 to about 3, from about 0.1 to about 2 mg, from about 0.1 to about 1 mg, from about 0.1 to about 0.9 mg, from about 0.1 to about 0.8 mg, from about 0.1 to about 0.7 mg, from about 0.1 to about 0.6 mg, from about 0.1 to about 0.5 mg, from about 0.1 to about 0.4 mg, from about 0.1 to about 0.3, or from about 0.1 to about 0.2 mg.

In some embodiments, the method as described herein further comprises administering to the human subject a pharmaceutical composition comprising the compound as described herein at a subsequent dose of from 0.1 to 950 mg, from 0.1 to 900 mg, from 0.1 to 850 mg, from 0.1 to 800 mg, from 0.1 to 750 mg, from 0.1 to 700 mg, from 0.1 to 650 mg, from 0.1 to 600 mg, from 0.1 to 550 mg, from 0.1 to 500 mg, from 0.1 to 450 mg, from 0.1 to 400 mg, from 0.1 to 350 mg, from 0.1 to 300 mg, from 0.1 to 250 mg, from 0.1 to 200 mg, from 0.1 to 150 mg, from 0.1 to 100 mg, from 0.1 to 95 mg, from 0.1 to 90 mg, from 0.1 to 85 mg, from 0.1 to 80 mg, from 0.1 to 75 mg, from 0.1 to 70 mg, from 0.1 to 65 mg, from 0.1 to 60 mg, from 0.1 to 55 mg, from 0.1 to 50 mg, from 0.1 to 45 mg, from 0.1 to 40 mg, from 0.1 to 35 mg, from 0.1 to 30 mg, from 0.1 to mg, from 0.1 to 25 mg, from 0.1 to 20 mg, from 0.1 to 10 mg, from 0.1 to 9 mg, from 0.1 to 8 mg, from 0.1 to 7 mg, from 0.1 to 6 mg, from 0.1 to 5 mg, from 0.1 to 4 mg, from 0.1 to 3, from 0.1 to 2 mg, from 0.1 to 1 mg, from 0.1 to 0.9 mg, from 0.1 to 0.8 mg, from 0.1 to 0.7 mg, from 0.1 to 0.6 mg, from 0.1 to 0.5 mg, from 0.1 to 0.4 mg, from 0.1 to 0.3, or from 0.1 to 0.2 mg.

In some embodiments, the method as described herein further comprises administering to the human subject a pharmaceutical composition comprising the compound as described herein at a subsequent dose of from about 1 to about 400 mg, from about 2 to about 400 mg, from about 3 to about 400 mg, from about 4 to about 400 mg, from about 5 to about 400 mg, from about 6 to about 400 mg, from about 7 to about 400 mg, from about 8 to about 400 mg, from about 9 to about 400 mg, from about 10 to about 400 mg, from about 20 to about 400 mg, from about 30 to about 400 mg, from about 40 to about 400 mg, from about 50 to about 400 mg, from about 60 to about 400 mg, from about 70 to about 400 mg, from about 80 to about 400 mg, from about 90 to about 400 mg, from about 100 to about 400 mg, from about 110 to about 400 mg, from about 120 to about 400 mg, from about 130 to about 400 mg, from about 140 to about 400 mg, from about 150 to about 400 mg, from about 160 to about 400 mg, from about 170 to about 400 mg, from about 180 to about 400 mg, from about 190 to about 400 mg, from about 200 to about 400 mg, from about 210 to about 400 mg, from about 220 to about 400 mg, from about 230 to about 400 mg, from about 240 to about 400 mg, from about 250 to about 400 mg, from about 260 to about 400 mg, from about 270 to about 400 mg, from about 280 to about 400 mg, from about 290 to about 400 mg, from about 300 to about 400 mg, from about 310 to about 400 mg, from about 320 to about 400 mg, from about 330 to about 400 mg, from about 340 to about 400 mg, from about 350 to about 400 mg, from about 360 to about 400 mg, from about 370 to about 400 mg, from about 380 to about 400 mg, or from about 390 to about 400 mg.

In some embodiments, the method as described herein further comprises administering to the human subject a pharmaceutical composition comprising the compound as described herein at a subsequent dose of from 1 to 400 mg, from 2 to 400 mg, from 3 to 400 mg, from 4 to 400 mg, from 5 to 400 mg, from 6 to 400 mg, from 7 to 400 mg, from 8 to 400 mg, from 9 to 400 mg, from 10 to 400 mg, from 20 to 400 mg, from 30 to 400 mg, from 40 to 400 mg, from 50 to 400 mg, from 60 to 400 mg, from 70 to 400 mg, from 80 to 400 mg, from 90 to 400 mg, from 100 to 400 mg, from 110 to 400 mg, from 120 to 400 mg, from 130 to 400 mg, from about 140 to 400 mg, from 150 to 400 mg, from about 160 to 400 mg, from 170 to 400 mg, from 180 to 400 mg, from 190 to 400 mg, from 200 to 400 mg, from 210 to 400 mg, from 220 to 400 mg, from 230 to 400 mg, from 240 to 400 mg, from 250 to 400 mg, from 260 to 400 mg, from 270 to 400 mg, from 280 to 400 mg, from 290 to 400 mg, from 300 to 400 mg, from 310 to 400 mg, from 320 to 400 mg, from 330 to 400 mg, from 340 to 400 mg, from 350 to 400 mg, from 360 to 400 mg, from 370 to 400 mg, from 380 to 400 mg, or from 390 to 400 mg.

In some embodiments, the method as described herein further comprises administering to the human subject a pharmaceutical composition comprising the compound as described herein at a subsequent dose of from about 10 to about 390 mg, from about 10 to about 380 mg, from about 10 to about 370 mg, from about 10 to about 360 mg, from about 10 to about 350 mg, from about 10 to about 340 mg, from about 10 to about 330 mg, from about 10 to about 320 mg, from about 10 to about 310 mg, from about 10 to about 300 mg, from about 10 to about 290 mg, from about 10 to about 280 mg, from about 10 to about 270 mg, from about 10 to about 260 mg, from about 10 to about 250 mg, from about 10 to about 240 mg, from about 10 to about 230 mg, from about 10 to about 220 mg, from about 10 to about 210 mg, from about 10 to about 200 mg, from about 10 to about 190 mg, from about 10 to about 180 mg, from about 10 to about 170 mg, from about 10 to about 160 mg, from about 10 to about 150 mg, from about 10 to about 140 mg, from about 10 to about 130 mg, from about 10 to about 120 mg, from about 10 to about 110 mg, from about 10 to about 90 mg, from about 10 to about 80 mg, from about 10 to about 70 mg, from about 10 to about 60 mg, from about 10 to about 50 mg, from about 10 to about 40 mg, from about 10 to about 30 mg, or from about 10 to about 20 mg.

In some embodiments, the method as described herein further comprises administering to the human subject a pharmaceutical composition comprising the compound as described herein at a subsequent dose of from 10 to 390 mg, from 10 to 380 mg, from 10 to 370 mg, from 10 to 360 mg, from 10 to 350 mg, from 10 to 340 mg, from 10 to 330 mg, from 10 to 320 mg, from 10 to 310 mg, from 10 to 300 mg, from 10 to 290 mg, from 10 to 280 mg, from 10 to 270 mg, from 10 to 260 mg, from 10 to 250 mg, from 10 to 240 mg, from 10 to 230 mg, from 10 to 220 mg, from 10 to 210 mg, from 10 to 200 mg, from 10 to 190 mg, from 10 to 180 mg, from 10 to 170 mg, from 10 to 160 mg, from 10 to 150 mg, from 10 to 140 mg, from 10 to 130 mg, from 10 to 120 mg, from 10 to 110 mg, from 10 to 90 mg, from 10 to 80 mg, from 10 to 70 mg, from 10 to 60 mg, from 10 to 50 mg, from 10 to 40 mg, from 10 to 30 mg, or from 10 to 20 mg.

In some embodiments mg, the method as described herein further comprises administering to the human subject a pharmaceutical composition comprising the compound as described herein at a subsequent dose of about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 151 mg, about 152 mg, about 153 mg, about 154 mg, about 155 mg, about 156 mg, about 157 mg, about 158 mg, about 159 mg, about 160 mg, about 161 mg, about 162 mg, about 163 mg, about 164 mg, about 165 mg, about 166 mg, about 167 mg, about 168 mg, about 169 mg, about 170 mg, about 171 mg, about 172 mg, about 173 mg, about 174 mg, about 175 mg, about 176 mg, about 177 mg, about 178 mg, about 179 mg, about 180 mg, about 181 mg, about 182 mg, about 183 mg, about 184 mg, about 185 mg, about 186 mg, about 187 mg, about 188 mg, about 189 mg, about 190 mg, about 191 mg, about 192 mg, about 193 mg, about 194 mg, about 195 mg, about 196 mg, about 197 mg, about 198 mg, about 199 mg, about 200 mg, about 201 mg, about 202 mg, about 203 mg, about 204 mg, about 205 mg, about 206 mg, about 207 mg, about 208 mg, about 209 mg, about 210 mg, about 211 mg, about 212 mg, about 213 mg, about 214 mg, about 215 mg, about 216 mg, about 217 mg, about 218 mg, about 219 mg, about 220 mg, about 221 mg, about 222 mg, about 223 mg, about 224 mg, about 225 mg, about 226 mg, about 227 mg, about 228 mg, about 229 mg, about 230 mg, about 231 mg, about 232 mg, about 233 mg, about 234 mg, about 235 mg, about 236 mg, about 237 mg, about 238 mg, about 239 mg, about 240 mg, about 241 mg, about 242 mg, about 243 mg, about 244 mg, about 245 mg, about 246 mg, about 247 mg, about 248 mg, about 249 mg, about 250 mg, about 251 mg, about 252 mg, about 253 mg, about 254 mg, about 255 mg, about 256 mg, about 257 mg, about 258 mg, about 259 mg, about 260 mg, about 261 mg, about 262 mg, about 263 mg, about 264 mg, about 265 mg, about 266 mg, about 267 mg, about 268 mg, about 269 mg, about 270 mg, about 271 mg, about 272 mg, about 273 mg, about 274 mg, about 275 mg, about 276 mg, about 277 mg, about 278 mg, about 279 mg, about 280 mg, about 281 mg, about 282 mg, about 283 mg, about 284 mg, about 285 mg, about 286 mg, about 287 mg, about 288 mg, about 289 mg, about 290 mg, about 291 mg, about 292 mg, about 293 mg, about 294 mg, about 295 mg, about 296 mg, about 297 mg, about 298 mg, about 299 mg, about 300 mg, about 301 mg, about 302 mg, about 303 mg, about 304 mg, about 305 mg, about 306 mg, about 307 mg, about 308 mg, about 309 mg, about 310 mg, about 311 mg, about 312 mg, about 313 mg, about 314 mg, about 315 mg, about 316 mg, about 317 mg, about 318 mg, about 319 mg, about 320 mg, about 321 mg, about 322 mg, about 323 mg, about 324 mg, about 325 mg, about 326 mg, about 327 mg, about 328 mg, about 329 mg, about 330 mg, about 331 mg, about 332 mg, about 333 mg, about 334 mg, about 335 mg, about 336 mg, about 337 mg, about 338 mg, about 339 mg, about 340 mg, about 341 mg, about 342 mg, about 343 mg, about 344 mg, about 345 mg, about 346 mg, about 347 mg, about 348 mg, about 349 mg, about 350 mg, about 351 mg, about 352 mg, about 353 mg, about 354 mg, about 355 mg, about 356 mg, about 357 mg, about 358 mg, about 359 mg, about 360 mg, about 361 mg, about 362 mg, about 363 mg, about 364 mg, about 365 mg, about 366 mg, about 367 mg, about 368 mg, about 369 mg, about 370 mg, about 371 mg, about 372 mg, about 373 mg, about 374 mg, about 375 mg, about 376 mg, about 377 mg, about 378 mg, about 379 mg, about 380 mg, about 381 mg, about 382 mg, about 383 mg, about 384 mg, about 385 mg, about 386 mg, about 387 mg, about 388 mg, about 389 mg, about 390 mg, about 391 mg, about 392 mg, about 393 mg, about 394 mg, about 395 mg, about 396 mg, about 397 mg, about 398 mg, about 399 mg, or 400 mg.

In some embodiments mg, the method as described herein further comprises administering to the human subject a pharmaceutical composition comprising the compound as described herein at a subsequent dose of 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, or 400 mg

In some embodiments, the method as described herein further comprises assessing tolerability or effectiveness of the pharmaceutical composition. In some embodiments, the subsequent dose is lower than the previous dose following an indication that administration of the previous dose is not tolerated. In some embodiments, the subsequent dose is the same as the previous dose following an indication that administration of the previous dose is effective. In some embodiments, the subsequent dose is lower than the previous dose following an indication that administration of the previous dose is effective. In some embodiments, the subsequent dose is higher than the previous dose following an indication that administration of the previous dose is not effective.

In some embodiments, the subsequent doses are administered at least 1, 2, 3, 4, 5, 6, 7, 8, 9, 10, 11, or 12 months after administration of the previous dose.

In some embodiments, the subsequent doses are administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hours after administration of the previous dose. In some embodiments, the subsequent doses are administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administration of the previous dose. In some embodiments, the subsequent doses are administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 weeks after administration of the previous dose. In some embodiments, the subsequent doses are administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after administration of the previous dose. In some embodiments, the subsequent doses are administered at least 1, 2, 3, 4, 5, 6, 7, 8, 9 or 10 years after administration of the previous dose.

In some embodiments, the subsequent doses are administered at the same interval. For example, every subsequent dose is administered at the interval of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hours after administration of the previous dose. In some embodiments, every subsequent dose is administered at the interval of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administration of the previous dose. In some embodiments, every subsequent dose is administered at the interval of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 weeks after administration of the previous dose. In some embodiments, every subsequent dose is administered at the interval of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after administration of the previous dose.

In some embodiments, the subsequent doses are administered at the different intervals.

In some embodiments, the dose frequency is maintained or reduced following an indication that the previous dose is effective. In some embodiments, the dose frequency is increased following an indication that the previous dose is not effective. In some embodiments, the method further comprises administrating at least one additional therapeutic agent or therapy. In some embodiments, the at least one additional therapeutic agent or therapy is administered at the same time as the dose. In some embodiments, the at least one additional therapeutic agent or therapy is administered prior to administration of the dose. In some embodiments, the at least one additional therapeutic agent or therapy is administered after administration of the dose.

### Loading and Maintenance Dose

In some embodiments, the method as described herein comprises administering to the human subject a pharmaceutical composition comprising the compound according to chemical structure (I) at least a first loading dose amount of from about 20 to about 80 mg and one or more maintenance doses, wherein the one or more maintenance doses contain a dose amount that is different from the first loading dose amount. In some embodiments, the method as described herein comprises administering to the human subject a pharmaceutical composition comprising the compound according to chemical structure (II) at a first dose amount of from about 30 to about 70 mg and one or more maintenance doses, wherein the one or more maintenance doses contain a dose amount that is different from the first loading dose amount.

In some embodiments, the first dose (also called first loading dose) is the first of one of more loading doses. In some embodiments, the first loading dose contains the compound at a dose amount of about 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, or 80 mg. In some embodiments, the first dose of the one or more loading doses contains the compound at a dose amount of from 30 to 40 mg. In some embodiments, the first dose of the one or more loading doses contains the compound at a dose amount of from 35 to 40 mg. In some embodiments, the first dose of the one or more loading doses contains the compound at a dose amount of from 30 to 45 mg. In some embodiments, the first dose of the one or more loading doses contains the compound at a dose amount of from 35 to 45 mg. In some embodiments, the first dose of the one or more loading doses contains the compound at a dose amount of from 30 to 50 mg. In some embodiments, the first dose of the one or more loading doses contains the compound at a dose amount of from 35 to 50 mg. In some embodiments, the first dose of the one or more loading doses contains the compound at a dose amount of from 30 to 35 mg. In some embodiments, the first dose of the one or more loading doses contains the compound at a dose amount of from 30 to 80 mg. In some embodiments, the first dose of the one or more loading doses contains the compound at a dose amount of from 30 to 70 mg. In some embodiments, the first dose of the one or more loading doses contains the compound at a dose amount of from 30 to 60 mg. In some embodiments, the first dose of the one or more loading doses contains the compound at a dose amount from 50 to 70 mg. In some embodiments, the first dose of the one or more loading doses contains the compound at a dose amount from 50 to 80 mg. In some embodiments, the first dose of the one or more loading doses contains the compound at a dose amount is from about 45 to 80 mg. In some embodiments, the first dose of the one or more loading doses contains the compound at a dose amount is from about 45 to 70 mg. In some embodiments, the first dose of the one or more loading doses contains the compound at a dose amount is from about 45 to 60 mg. In some embodiments, the first dose of the one or more loading doses contains the compound at a dose amount of about 30 mg. In some embodiments, the first dose of the one or more loading doses contains the compound at a dose amount of about 35 mg. In some embodiments, the first dose of the one or more loading doses contains the compound at a dose amount of about 40 mg. In some embodiments, the first dose of the one or more loading doses contains the compound at a dose amount of about 45 mg. In some embodiments, the first dose of the one or more loading doses contains the compound at a dose amount of about 50 mg. In some embodiments, the first dose of the one or more loading doses contains the compound at a dose amount of about 60 mg. In some embodiments, the first dose of the one or more loading doses contains the compound at a dose amount of about 70 mg. In some embodiments, the first dose of the one or more loading doses contains the compound at a dose amount of about 80 mg.

In some embodiments, the method comprises administering only one first dose of the one or more loading doses. In some embodiments, the method comprises administering two doses of the loading dose. In some embodiments, the method comprises administering three doses of the loading dose. In some embodiments, the method comprises administering four doses of the loading dose. In some embodiments, the method comprises administering five doses of the loading dose. In some embodiments, the method comprises administering at least two doses of the loading dose. In some embodiments, the method comprises administering at least three doses of the loading dose. In some embodiments, the method comprises administering at least four doses of the loading dose. In some embodiments, the method comprises administering from 2 to 5 doses of the loading dose. In some embodiments, the method comprises administering from 2 to 4 doses of the loading dose. In some embodiments, the method comprises administering from 2 to 3 doses of the loading dose. In some embodiments, the method comprises administering two doses of the loading dose, wherein the loading dose contains the compound at a dose amount of about 30 mg. In some embodiments, the method comprises administering three doses of the loading dose, wherein the loading dose contains the compound at a dose amount of about 30 mg. In some embodiments, the method comprises administering four doses of the loading dose, wherein the loading dose contains the compound at a dose amount of about 30 mg. In some embodiments, the method comprises administering two doses of the loading dose, wherein the loading dose contains the compound at a dose amount of about 45 mg. In some embodiments, the method comprises administering three doses of the loading dose, wherein the loading dose contains the compound at a dose amount of about 45 mg. In some embodiments, the method comprises administering two doses of the loading dose, wherein the loading dose contains the compound at a dose amount of about 60 mg. In some embodiments, the method comprises administering three doses of the loading dose, wherein the loading dose contains the compound at a dose amount of about 60 mg. In some embodiments, the method comprises administering two doses of the loading dose, wherein the loading dose contains the compound at a dose amount of about 70 mg. In some embodiments, the method comprises administering three doses of the loading dose, wherein the loading dose contains the compound at a dose amount of about 70 mg. In some embodiments, the method comprises administering two doses of the loading dose, wherein the loading dose contains the compound at a dose amount of about 80 mg. In some embodiments, the method comprises administering three doses of the loading dose, wherein the loading dose contains the compound at a dose amount of about 80 mg.

In some embodiments, a first loading dose of the one or more loading dose doses is administered at least 1, 2, 3, 4, 5, 6, 7, 8, 9, 10, 11, or 12 months after administration of the first dose. In some embodiments, a first loading dose of the one or more loading dose doses is administered at least 1, 2, 3, 4, 5, 6, 7, or 8 weeks after administration of the first dose. In some embodiments, a first loading dose of the one or more loading dose doses is administered about 4 months after the first dose is administered. In some embodiments, the one or more loading dose doses is administered at least 1, 2, 3, 4, 5, 6, 7, 8, 9, 10, 11, or 12 months after administration of a dose immediately prior. In some embodiments, the one or more loading dose doses is administered about 2 months after administration of a dose immediately prior. In some embodiments, the one or more loading dose doses is administered about 3 months after administration of a dose immediately prior. In some embodiments, the one or more loading dose doses is administered about 4 months after administration of a dose immediately prior. In some embodiments, the one or more loading dose doses is administered about 5 months after administration of a dose immediately prior. In some embodiments, the one or more loading dose doses is administered at least 1, 2, 3, 4, 5, 6, 7, or 8 weeks after administration of a dose immediately prior. In some embodiments, each loading dose of the one or more loading dose doses is administered about 4 months after the human subject has been administered a dose immediately prior. In some embodiments, dose frequency is maintained or reduced following an indication that the previous dose is effective. In some embodiments, dose frequency is increased following an indication that the previous dose is not effective.

In some embodiments, the one or more maintenance doses are higher than the first dose. In some embodiments, the one or more maintenance doses are lower than the first dose. In some embodiments, the one or more maintenance doses contain the compound at a dose amount of about 10, 15, 20, 25, 30, 35, 40, 45, 50, 55, 60, 65, 70, 75, or 80 mg. In some embodiments, the one or more maintenance doses contain the compound at a dose amount of 10, 11, 12, 13, 14, 15, 16, 17, 18, 19, 20, 22.5, 25, 27.5, 30, 32.5, 35, 37.5, 40, 42.5, 45, 47.5, 50, 52.5, 55, 57.5, 60, 62.5, 65, 67.5, 70, 72.5, 75, 77.5, 80, 82.5, 85, 87.5, 90, 92.5, 95, 97.5, or 100 mg. In some embodiments, the one or more maintenance doses contain the compound at a dose amount of from 10 to 45 mg. In some embodiments, the one or more maintenance doses contain the compound at a dose amount of from 15 to 45 mg. In some embodiments, the one or more maintenance doses contain the compound at a dose amount of from 20 to 45 mg. In some embodiments, the one or more maintenance doses contain the compound at a dose amount of from 25 to 45 mg. In some embodiments, the one or more maintenance doses contain the compound at a dose amount of from 30 to 45 mg. In some embodiments, the one or more maintenance doses contain the compound at a dose amount of from 35 to 45 mg. In some embodiments, the one or more maintenance doses contain the compound at a dose amount of from 10 to 40 mg. In some embodiments, the one or more maintenance doses contain the compound at a dose amount of from 15 to 40 mg. In some embodiments, the one or more maintenance doses contain the compound at a dose amount of from 20 to 40 mg. In some embodiments, the one or more maintenance doses contain the compound at a dose amount of from 25 to 40 mg. In some embodiments, the one or more maintenance doses contain the compound at a dose amount of from 30 to 40 mg. In some embodiments, the one or more maintenance doses contain the compound at a dose amount of from 35 to 40 mg. In some embodiments, the one or more maintenance doses contain the compound at a dose amount of from 10 to 35 mg. In some embodiments, the one or more maintenance doses contain the compound at a dose amount of from 15 to 35 mg. In some embodiments, the one or more maintenance doses contain the compound at a dose amount of from 20 to 35 mg. In some embodiments, the one or more maintenance doses contain the compound at a dose amount of from 25 to 35 mg. In some embodiments, the one or more maintenance doses contain the compound at a dose amount of from 30 to 35 mg. In some embodiments, the one or more maintenance doses contain the compound at a dose amount of from 10 to 30 mg. In some embodiments, the one or more maintenance doses contain the compound at a dose amount of from 15 to 30 mg. In some embodiments, the one or more maintenance doses contain the compound at a dose amount of from 20 to 30 mg. In some embodiments, the one or more maintenance doses contain the compound at a dose amount of from 25 to 30 mg. In some embodiments, the one or more maintenance doses contain the compound at a dose amount of from 10 to 25 mg. In some embodiments, the one or more maintenance doses contain the compound at a dose amount of from 15 to 25 mg. In some embodiments, the one or more maintenance doses contain the compound at a dose amount of from 20 to 25 mg. In some embodiments, the one or more maintenance doses contain the compound at a dose amount of from 10 to 20 mg. In some embodiments, the one or more maintenance doses contain the compound at a dose amount of from 15 to 20 mg.

In some embodiments, the one or more maintenance doses contain the compound at a dose amount of about 20 mg. In some embodiments, the one or more maintenance doses contain the compound at a dose amount of about 25 mg. In some embodiments, the one or more maintenance doses contain the compound at a dose amount of about 30 mg. In some embodiments, the one or more maintenance doses contain the compound at a dose amount of about 35 mg. In some embodiments, the one or more maintenance doses contain the compound at a dose amount of about 40 mg. In some embodiments, the one or more maintenance doses contain the compound at a dose amount of about 45 mg. In some embodiments, the one or more maintenance doses contain the compound at a dose amount of about 50 mg. In some embodiments, the one or more maintenance doses contain the compound at a dose amount of about 55 mg. In some embodiments, the one or more maintenance doses contain the compound at a dose amount of about 60 mg. In some embodiments, the one or more maintenance doses contain the compound at a dose amount of about 65 mg. In some embodiments, the one or more maintenance doses contain the compound at a dose amount effective to reduce seizure frequency and/or improve cognition and/or behavior for over one year of dosing compared to a subject not treated with the compound. In some embodiments, the one or more maintenance doses contain the compound at a dose amount effective to reduce seizure frequency and/or improve cognition and/or behavior for over one year of dosing compared to a subject administered the first dose but not administered the one or more maintenance doses.

In some embodiments, the method comprises administering at least two maintenance doses. In some embodiments, the method comprises administering at least 3, 4, 5, 6, 7, 8, 9 or 10 maintenance doses. In some embodiments, the method comprises administering 2 maintenance doses. In some embodiments, the method comprises administering 3 maintenance doses. In some embodiments, the method comprises administering 4, 5, 6, 7, 8, 9, or 10 maintenance doses. In some embodiments, the maintenance doses are administered every two, Three, or four months. In some embodiments, the maintenance doses are administered every four months.

In some embodiments, the one or more maintenance doses contain the compound at a dose amount that is lower than the first dose amount. In some embodiments, the one or more maintenance doses is lower than the first dose following an indication that administration of the first dose is not tolerated. In some embodiments, the one or more maintenance doses is lower than the first dose following an indication that administration of the first dose is effective. In some embodiments, the one or more maintenance doses contain the compound at a dose amount that is at least about 10%, 20%, 30%, 40%, 50%, 60%, 70%, or 80% lower than the first dose amount. In some embodiments, the one or more maintenance doses contain the compound at a dose amount that is at least about 5 mg, 10 mg, 15 mg, 20 mg, 30 mg, 35 mg, 40 mg, or 45 mg lower than the first dose amount. In some embodiments, the one or more maintenance doses contain the compound at a dose amount that is higher than the first dose amount. In some embodiments, the one or more maintenance doses is higher than the first dose following an indication that administration of the first dose is tolerated. In some embodiments, the one or more maintenance doses is higher than the first dose following an indication that administration of the first dose is not effective. In some embodiments, the one or more maintenance doses contain the compound at a dose amount that is at least about 10%, 20%, 30%, 40%, 50%, 60%, 70%, or 80% higher than the first dose amount. In some embodiments, the one or more maintenance doses contain the compound at a dose amount that is at least about 5 mg, 10 mg, 15 mg, 20 mg, 30 mg, 50 mg, or 70 mg higher than the first dose amount.

In some embodiments, a first maintenance dose of the one or more maintenance doses is administered at least 1, 2, 3, 4, 5, 6, 7, 8, 9, 10, 11, or 12 months after administration of the first dose. In some embodiments, a first maintenance dose of the one or more maintenance doses is administered at least 1, 2, 3, 4, 5, 6, 7, or 8 weeks after administration of the first dose. In some embodiments, a first maintenance dose of the one or more maintenance doses is administered about 4 months after the first dose is administered. In some embodiments, the one or more maintenance doses is administered at least 1, 2, 3, 4, 5, 6, 7, 8, 9, 10, 11, or 12 months after administration of a dose immediately prior. In some embodiments, the one or more maintenance doses is administered about 2 months after administration of a dose immediately prior. In some embodiments, the one or more maintenance doses is administered about 3 months after administration of a dose immediately prior. In some embodiments, the one or more maintenance doses is administered about 4 months after administration of a dose immediately prior. In some embodiments, the one or more maintenance doses is administered about 5 months after administration of a dose immediately prior. In some embodiments, the one or more maintenance doses is administered at least 1, 2, 3, 4, 5, 6, 7, or 8 weeks after administration of a dose immediately prior. In some embodiments, each maintenance dose of the one or more maintenance doses is administered about 4 months after the human subject has been administered a dose immediately prior. In some embodiments, dose frequency is maintained or reduced following an indication that the previous dose is effective. In some embodiments, dose frequency is increased following an indication that the previous dose is not effective.

In some embodiments, the method comprises administering to the human subject a pharmaceutical composition comprising the compound according to chemical structure (I) or (II), wherein the method comprises administering to the human subject the pharmaceutical composition at a first dose amount of from about 30 to 80 mg and one or more maintenance doses at a dose amount of from about 30 to 50 mg, wherein the one or more maintenance doses contain a dose amount that is different from the first dose amount. In some embodiments, the method comprises administering to the human subject the pharmaceutical composition at a first dose amount of from about 30 to 70 mg and one or more maintenance doses at a dose amount of from about 30 to 50 mg, wherein the one or more maintenance doses contain a dose amount that is different from the first dose amount. In some embodiments, the method comprises administering to the human subject the pharmaceutical composition at a first dose amount of from about 40 to 70 mg and one or more maintenance doses at a dose amount of from about 30 to 45 mg, wherein the one or more maintenance doses contain a dose amount that is different from the first dose amount. In some embodiments, the method comprises administering to the human subject the pharmaceutical composition at a first dose amount of from about 45 to 70 mg and one or more maintenance doses at a dose amount of from about 30 to 45 mg, wherein the one or more maintenance doses contain a dose amount that is different from the first dose amount. In some embodiments, the method comprises administering to the human subject the pharmaceutical composition at a first dose amount of at least 45 mg and one or more maintenance doses at a dose amount of at least 30 mg. In some embodiments, the method comprises administering to the human subject the pharmaceutical composition at a first dose amount of about 45 mg and one or more maintenance doses at a dose amount of about 30 mg. In some embodiments, the method comprises administering to the human subject the pharmaceutical composition at a first dose amount of about 50 mg and one or more maintenance doses at a dose amount of about 35 mg. In some embodiments, the method comprises administering to the human subject the pharmaceutical composition at a first dose amount of about 55 mg and one or more maintenance doses at a dose amount of about 40 mg. In some embodiments, the method comprises administering to the human subject the pharmaceutical composition at a first dose amount of about 60 mg and one or more maintenance doses at a dose amount of about 45 mg. In some embodiments, the method comprises administering to the human subject the pharmaceutical composition at a first dose amount of about 70 mg and one or more maintenance doses at a dose amount of about 30 mg. In some embodiments, the method comprises administering to the human subject the pharmaceutical composition at a first dose amount of about 70 mg and one or more maintenance doses at a dose amount of about 45 mg. In some embodiments, the method comprises administering to the human subject the pharmaceutical composition at a first dose amount of about 70 mg and one or more maintenance doses at a dose amount of from about 30 mg to about 60 mg. In some embodiments, the method comprises administering to the human subject the pharmaceutical composition at a first dose amount of about 70 mg and one or more maintenance doses at a dose amount of from about 30 mg to about 50 mg. In some embodiments, the method comprises administering to the human subject the pharmaceutical composition at a first dose amount of about 70 mg and one or more maintenance doses at a dose amount of from about 30 mg to about 45 mg. In some embodiments, the method comprises administering to the human subject the pharmaceutical composition at a first dose amount of about 70 mg and one or more maintenance doses at a dose amount of from about 30 mg to about 40 mg. In some embodiments, the method comprises administering to the human subject the pharmaceutical composition at a first dose amount of about 70 mg and one or more maintenance doses at a dose amount of from about 30 mg to about 35 mg. The methods disclosed herein can (i) reduce seizure frequency and/or (ii) improve cognition and/or behavior after at least 6 months, 9 months, or 1 year dosing of the maintenance dose as compared to a control, such as observed natural history of the disease.

In some embodiments, the method as described herein comprises administering to the human subject a pharmaceutical composition comprising the compound according to chemical structure (I), or in some cases, the chemical structure (II), wherein the method comprises administering to the human subject the pharmaceutical composition comprising the compound at a first dose amount of from 30 to 70 mg and one or more maintenance doses, wherein the one or more maintenance doses contain a dose amount that is different from the first dose amount. In some embodiments, the pharmaceutical composition comprising the compound is administered into the intrathecal space of the human subject. In some embodiments, the pharmaceutical composition is administered into the cerebrospinal fluid of the human subject. In some embodiments, the pharmaceutical composition is administered into the brain of the human subject. In some embodiments, the pharmaceutical composition is administered into the cerebrospinal fluid in the brain of the human subject. In some embodiments, the pharmaceutical composition is administered as a bolus injection. In some embodiments, the method comprises administering the pharmaceutical composition as a bolus injection over 1 to 60 minutes, 1 to 50 minutes, 1 to 40 minutes, 1 to 30 minutes, 1 to 20 minutes, 1 to 10 minutes, 1 to 5 minutes, or 1 to 3 minutes. In some embodiments, the method comprises administering the pharmaceutical composition as a bolus injection using a spinal anesthesia needle. In some embodiments, the pharmaceutical composition is administered by infusion with a delivery pump. In some embodiments, the pharmaceutical composition is administered by intracerebroventricular injection. In some embodiments, the pharmaceutical composition is administered by intrathecal injection. In some embodiments, the pharmaceutical composition is administered by lumbar injection.

In some embodiments, the methods herein provide administering to the human subject a pharmaceutical composition comprising the compound according to chemical structure (I), or in some cases, the chemical structure (II), wherein the pharmaceutical composition comprises the compound at a first dose amount of from 30 to 50 mg and one or more maintenance doses, wherein the one or more maintenance doses contain a dose amount that is different from the first dose amount. In some embodiments, the pharmaceutical composition comprises a pharmaceutically acceptable excipient, carrier or diluent. In some embodiments, the pharmaceutical composition is a solution. In some embodiments, the compound is present in the pharmaceutical composition at a concentration of from 0.1 mg/mL to 250 mg/mL. In some embodiments, the compound is present in the first dose at a concentration of about 0.1 mg/mL, 0.5 mg/mL, 1 mg/mL, 2 mg/mL, 2.5 mg/mL, 3 mg/mL, 4 mg/mL, 5 mg/mL, 6 mg/mL, 7 mg/mL, 8 mg/mL, 9 mg/mL, or 10 mg/mL. In some embodiments, the compound is present in the maintenance dose at a concentration of about 0.1 mg/mL, 0.5 mg/mL, 1 mg/mL, 2 mg/mL, 2.5 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, or 20 mg/mL.

In some embodiments, the pharmaceutical composition comprising the first dose is a solution. In some embodiments, the compound is dissolved or suspended in the solution and the first dose has a volume of 5 mL or higher. In some embodiments, the compound is dissolved or suspended in a solution and the first dose has a volume of from 5 to 50 mL. In some embodiments, the compound is dissolved or suspended in a solution and the first dose has a volume of from 5 to 25 mL. In some embodiments, the compound is dissolved or suspended in a solution and the first dose has a volume of from 5 to 10 mL. In some embodiments, the compound is dissolved or suspended in a solution and the first dose has a volume of 5 mL. In some embodiments, the compound is dissolved or suspended in a solution and the first dose has a volume of 6 mL. In some embodiments, the compound is dissolved or suspended in a solution and the first dose has a volume of 7 mL. In some embodiments, the compound is dissolved or suspended in a solution and the first dose has a volume of 8 mL. In some embodiments, the compound is dissolved or suspended in a solution and the first dose has a volume of 9 mL. In some embodiments, the compound is dissolved or suspended in a solution and the first dose has a volume of 10 mL. In some embodiments, the compound is dissolved or suspended in a solution and the first dose has a volume of 11 mL. In some embodiments, the compound is dissolved or suspended in a solution and the first dose has a volume of 12 mL. In some embodiments, the compound is dissolved or suspended in a solution and the first dose has a volume of 13 mL. In some embodiments, the compound is dissolved or suspended in a solution and the first dose has a volume of 14 mL. In some embodiments, the compound is dissolved or suspended in a solution and the first dose has a volume of 15 mL. In some embodiments, the compound is dissolved or suspended in a solution and the first dose has a volume of 17 mL. In some embodiments, the compound is dissolved or suspended in a solution and the first dose has a volume of 20 mL. In some embodiments, the compound is dissolved or suspended in a solution and the first dose has a volume of 25 mL. In some embodiments, the compound is dissolved or suspended in a solution and the first dose has a volume of 30 mL.

In some embodiments, the pharmaceutical composition comprising the one or more maintenance doses is a solution. In some embodiments, the compound is dissolved or suspended in a solution and each of the one or more maintenance doses has a volume of 10 mL or higher. In some embodiments, the compound is dissolved or suspended in a solution and each of the one or more maintenance doses has a volume of from 10 to 50 mL. In some embodiments, the compound is dissolved or suspended in a solution and the first dose has a volume of from 5 to 25 mL. In some embodiments, the compound is dissolved or suspended in a solution and the first dose has a volume of from 5 to 10 mL. In some embodiments, the compound is dissolved or suspended in a solution and the first dose has a volume of 5 mL. In some embodiments, the compound is dissolved or suspended in a solution and the first dose has a volume of 6 mL. In some embodiments, the compound is dissolved or suspended in a solution and the first dose has a volume of 7 mL. In some embodiments, the compound is dissolved or suspended in a solution and the first dose has a volume of 8 mL. In some embodiments, the compound is dissolved or suspended in a solution and the first dose has a volume of 9 mL. In some embodiments, the compound is dissolved or suspended in a solution and each of the one or more maintenance doses has a volume of 10 mL. In some embodiments, the compound is dissolved or suspended in a solution and the first dose has a volume of 11 mL. In some embodiments, the compound is dissolved or suspended in a solution and the first dose has a volume of 12 mL. In some embodiments, the compound is dissolved or suspended in a solution and the first dose has a volume of 13 mL. In some embodiments, the compound is dissolved or suspended in a solution and the first dose has a volume of 14 mL. In some embodiments, the compound is dissolved or suspended in a solution and each of the one or more maintenance doses has a volume of 15 mL. In some embodiments, the compound is dissolved or suspended in a solution and the first dose has a volume of 17 mL. In some embodiments, the compound is dissolved or suspended in a solution and each of the one or more maintenance doses has a volume of 20 mL. In some embodiments, the compound is dissolved or suspended in a solution and each of the one or more maintenance doses has a volume of 25 mL. In some embodiments, the compound is dissolved or suspended in a solution and each of the one or more maintenance doses has a volume of 30 mL.

In some embodiments, the pharmaceutical composition is a solution. In some cases, the solution comprises a pharmaceutically acceptable excipient, carrier or diluent. In some embodiments, the pharmaceutical composition comprises from 0.1 mL to 50 mL of a diluent, wherein the compound is solubilized or diluted in the diluent. In some embodiments, the pharmaceutical composition comprises about 0.1, 0.5, 1, 2, 2.5, 3, 4, 5, 6, 7, 8, 9, 10, 11, 12, 13, 14, 15, 16, 17, 18, 19, 20, 25, 30, 35, 40, 45 or 50 mL of the diluent. In some embodiments, the pharmaceutical composition comprises 1 mL to 20 mL of the diluent, 2 mL to 10 mL of the diluent or 1 mL to 5 mL of the diluent. In some embodiments, the diluent comprises a cerebral spinal fluid (CSF) sample from the human subject or an artificial cerebral spinal fluid (aCSF) solution. In some embodiments, the compound is solubilized or diluted in an isotonic solution. In some embodiments, the isotonic solution is a CSF sample from the human subject. In some embodiments, the compound is solubilized or diluted in the pharmaceutical composition in a phosphate-buffered solution with at least pH 5.8. In some embodiments, the compound is solubilized or diluted in the pharmaceutical composition in a phosphate-buffered (pH 6.6-7.6) solution. In some embodiments, the buffer comprises 25-250 mM NaCl, 0.1-20 mM KCl, 0.1-50 mM NaH₂PO₄, 0.1-50 mM NaH₂PO₄, 0.1-50 mM CaCl₂, 0.1-50 mM MgCl₂ or a combination thereof. In some embodiments, the buffer comprises 150 mM NaCl, 3.0 mM KCl, 0.7 mM NaH₂PO₄, 0.3 mM NaH₂PO₄, 0.79 mM MgCl₂, and 1.4 mM CaCl₂. In some embodiments, the buffer comprises 1-100 mM NaHCO₃, 1-100 mM KHCO₃, or a combination thereof. In some embodiments, the buffer comprises carbohydrates. In some embodiments, the buffer comprises D-glucose. In some embodiments, the buffer comprises 1-100 mM D-glucose. In some embodiments, the buffer comprises an antioxidant. In some embodiments, the antioxidant is t-butylhydroxyquinoline (TBHQ), butylated hydroxytoluene (BHT), butylated hydroxyanisole (BHA), vitamin E, or any combination thereof. In some embodiments, the pharmaceutical formulation does not comprise a preservative.

### Dravet Syndrome and Other Related Diseases

The terms “condition,” “diseases,” and “disorders” are used herein interchangeably in its broadest sense and include susceptibilities. In some embodiments, the disease or condition is Dravet Syndrome. In some embodiments, the method reduces or ameliorates at least one symptom of Dravet Syndrome in the human subject. In some embodiments, the symptom of Dravet Syndrome is a seizure. In some embodiments, the administration reduces or ameliorates seizure frequency, seizure intensity, or seizure duration.

Dravet syndrome (DS), otherwise known as severe myoclonic epilepsy of infancy (SMEI), is an epileptic encephalopathy presenting in the first year of life. Dravet syndrome is an increasingly recognized epileptic encephalopathy in which the clinical diagnosis is supported by the finding of sodium channel gene mutations in approximately 70-80% of patients. DS is a severe and progressive developmental and epileptic encephalopathy that is characterized by high seizure frequency and severity, intellectual disability, and a high risk of sudden unexpected death in epilepsy. Mutations of ion channel genes play a major role in the pathogenesis of a range of epilepsy syndromes, resulting in some epilepsies being regarded as channelopathies. Voltage-gated sodium channels (VGSCs) play an essential role in neuronal excitability; therefore, it is not surprising that many mutations associated with DS have been identified in the gene encoding a VGSC subunit. The disease is described by, e.g., Mulley, et al., 2005, and the disease description at OMIM #607208 (Online Mendelian Inheritance in Man, Johns Hopkins University, 1966-2015), both incorporated by reference herein.

Between 70% and 80% of patients carry sodium channel al subunit gene (SCN1A) abnormalities, and truncating mutations account for about 40%, and have a significant correlation with an earlier age of seizures onset. Sequencing mutations are found in about 70% of cases and comprise truncating (40%) and missense mutations (40%) with the remaining being splice-site changes. Most mutations are de novo, but familial mutations occur in 5-10% of cases and are usually missense in nature. The remaining SCN1A mutations comprise splice-site and missense mutations, most of which fall into the pore-forming region of the sodium channel. At present, over 500 mutations have been associated with DS and are randomly distributed along the gene (Mulley, et al., Neurol. 2006, 67, 1094-1095).

The SCN1A gene is located in the cluster of sodium channel genes on human chromosome 2q24 and encodes the α-pore forming subunits known as Na_{V}1.1 of the neuronal voltage-gated sodium channel. The SCN1A gene spans approximately 100 kb of genomic DNA and comprises 26 exons. The Na_{V}1.1 protein consists of four domains, each with six-transmembrane segments. Two splice variants have been identified that result in a long and short isoform that differ in the presence or absence of 11 amino acids in the cytoplasmic loop between domains 1 and 2, in exon 11 (Miller, et al., 1993-2015, and Mulley, et al., 2005, 25, 535-542, of which entire content is incorporated herein by reference). In some aspects, the variants or homologs have at least 80%, 85%, 90%, 95%, 96%, 97%, 98%, 99% or 100% nucleotide sequence identity across the whole sequence or a portion of the sequence (e.g., a 50, 100, 150 or 200 continuous nucleotide portion) compared to a naturally occurring SCN1A gene. In some embodiments, the SCN1A gene is substantially identical to the gene identified by the Ensembl reference number ENSG00000144285 or a variant or homolog having substantial identity thereto.

Alternative splicing events in SCN1A gene can lead to non-productive mRNA transcripts which in turn can lead to aberrant protein expression, and therapeutic agents which can target the alternative splicing events in SCN1A gene can modulate the expression level of functional proteins in DS patients and/or inhibit aberrant protein expression. Such therapeutic agents can be used to treat a condition characterized by Na_{V}1.1 protein deficiency.

One of the alternative splicing events that can lead to non-productive mRNA transcripts is the inclusion of an extra exon in the mRNA transcript that can induce nonsense-mediated mRNA decay. The present disclosure provides compositions and methods for modulating alternative splicing of SCN1A to increase the production of protein-coding mature mRNA, and thus, translated functional Na_{V}1.1 protein. These compositions and methods include compounds that can cause exon skipping and promote constitutive splicing of SCN1A pre-mRNA. In various embodiments, functional Na_{V}1.1 protein can be increased using the methods of the disclosure to treat a condition characterized by Na_{V}1.1 protein deficiency.

In some cases, the disease or condition is SMEB.

In some cases, the disease or condition is GEFS+.

In some cases, the disease or condition is a Febrile seizure (e.g., Febrile seizures, familial, 3A).

In some cases, the disease or condition is autism (also known as autism spectrum disorder or ASD).

In some cases, the disease or condition is migraine (e.g., migraine, familial hemiplegic, 3).

In some cases, the disease or condition is Alzheimer's disease.

In some embodiments, the disease or condition is SMEB. In some embodiments, the disease or condition is GEFS+. In some embodiments, the disease or condition is a Febrile seizure (e.g., Febrile seizures, familial, 3A). In some embodiments, the disease or condition is autism (also known as autism spectrum disorder or ASD). In some embodiments, the disease or condition is migraine (e.g., migraine, familial hemiplegic, 3). In some embodiments, the disease or condition is Alzheimer's disease. In some embodiments, the disease or condition is SCN2A encephalopathy. In some embodiments, the disease or condition is SCN8A encephalopathy. In some embodiments, the disease or condition is SCN5A arrhythmia.

In some embodiments, the disease or condition is characterized by a mutation in Na_{V}1.1 (a protein encoded by the SCN1A gene). “Na_{V}1.1,” also known as the sodium channel, voltage-gated, type I, alpha subunit (SCN1A), as used herein, refers to a protein which in humans is encoded by the SCN1A gene. Na_{V}1.1 includes any of the recombinant or naturally occurring forms of Na_{V}1.1 protein or variants or homologs thereof that maintain Na_{V}1.1 activity, (e.g., within at least 50%, 80%, 90%, 95%, 96%, 97%, 98%, 99% or 100% activity compared to Na_{V}1.1). In some aspects, the variants or homologs have at least 80%, 85%, 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring Na_{V}1.1 protein. In some embodiments, the Na_{V}1.1 protein is substantially identical to the protein identified by the UniProt reference number P35498 or a variant or homolog having substantial identity thereto.

In some instances, the mutation is a loss-of-function mutation in Naᵥ1.1. In some cases, the loss-of-function mutation in Na_{V}1.1 comprises one or more mutations that decreases or impairs the function of Na_{V}1.1 (e.g., by 10%, 20%, 30%, 40%, 50%, 60%, 70%, 80%, 90%, 95%, or more) relative to the function of a wild-type Naᵥ1.1. In some cases, the loss-of-function mutation in Naᵥ1.1 comprises one or more mutations that result in a disease phenotype. Exemplary loss-of-function mutations include, but are not limited to, R859C, T875M, V1353L, I1656M, R1657C, A1685V, M1841T, and R1916G.

In other instances, the mutation is a gain-of-function mutation in Na_{V}1.1. In such cases, the gain-of-function mutation comprises one or more mutations that prolongs activation of Naᵥ1.1 (e.g., by 10%, 20%, 30%, 40%, 50%, 60%, 70%, 80%, 90%, 95%, or more) relative to the function of a wild-type Naᵥ1.1. In such cases, the gain-of-function mutation in Naᵥ1.1 comprises one or more mutations that result in a disease phenotype. Exemplary gain-of-function mutations include, but are not limited to, D188V, W1204R, R1648H, and D1866Y.

In some embodiments, the disease or condition is an encephalopathy. In some cases, the encephalopathy is characterized by a loss-of-function mutation in Na_{V}1.1.

In some embodiments, the encephalopathy is epileptic encephalopathy. Exemplary epileptic encephalopathies include, but are not limited to, Dravet Syndrome (DS) (also known as severe myoclonic epilepsy of infancy or SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); early infantile SCN1A encephalopathy; early infantile epileptic encephalopathy (EIEE); autism; malignant migrating partial seizures of infancy; or sick sinus syndrome 1. In some embodiments, the disease or condition is epileptic encephalopathy, optionally selected from Dravet Syndrome (DS) (also known as severe myoclonic epilepsy of infancy or SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); and sick sinus syndrome 1.

In some instances, GEFS+ is epilepsy, generalized, with febrile seizures plus, type 2.

In some instances, the Febrile seizure is Febrile seizures, familial, 3A.

In some instances, SMEB is SMEB without generalized spike wave (SMEB-SW), SMEB with-out myoclonic seizures (SMEB-M), SMEB lacking more than one feature of SMEI (SMEB-O), or intractable childhood epilepsy with generalized tonic-clonic seizures (ICEGTC).

In some embodiments, GEFS+ is epilepsy, generalized, with febrile seizures plus, type 2. In some embodiments, the Febrile seizure is Febrile seizures, familial, 3A. In some embodiments, SMEB is SMEB without generalized spike wave (SMEB-SW), SMEB without myoclonic seizures (SMEB-M), SMEB lacking more than one feature of SMEI (SMEB-O), or intractable childhood epilepsy with generalized tonic-clonic seizures (ICEGTC).

In some embodiments, the diseases or conditions characterized by a loss-of-function mutation in Na_{V}1.1 include, but are not limited to, Dravet Syndrome (DS) (also known as SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); autism; or malignant migrating partial seizures of infancy.

In related embodiments, the method is a method of using a compound to decrease the expression of a protein or functional RNA. In some embodiments, a compound is used to decrease the expression of Na_{V}1.1 protein in cells of a subject having an NMD-inducing exon (NIE) containing pre-mRNA encoding Na_{V}1.1 protein. In some embodiments, the human subject has a gain-of-function mutation in Na_{V}1.1, e.g., migraine. In some embodiments, a compound is used to decrease the expression of Na_{V}1.1 protein in cells of a subject, the human subject has a gain-of-function mutation in Na_{V}1.1, e.g., migraine, familial hemiplegic, 3.

In some embodiments, the level of mRNA encoding Na_{V}1.1 protein is decreased 1.1 to 10-fold, when compared to the amount of mRNA encoding Na_{V}1.1 protein that is produced in a control cell, e.g., one that is not treated with the antisense oligomer or one that is treated with an antisense oligomer that does not bind to the targeted portion of the SCN1A NIE containing pre-mRNA.

In some embodiments, the disease or condition is a Na_{V}1.1 genetic epilepsy. The Na_{V}1.1 genetic epilepsy can include a loss-of-function mutation in Naᵥ1.1 or a gain-of-function mutation in Naᵥ1.1. In some cases, the Na_{V}1.1 genetic epilepsy includes one or more hereditary mutations. In other cases, the Na_{V}1.1 genetic epilepsy includes one or more de novo mutations. In some cases, the Na_{V}1.1 genetic epilepsy includes Dravet Syndrome (DS) (also known as severe myoclonic epilepsy of infancy or SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; early infantile SCN1A encephalopathy; early infantile epileptic encephalopathy (EIEE); sudden unexpected death in epilepsy (SUDEP); or malignant migrating partial seizures of infancy. In some cases, the Naᵥ1.1 genetic epilepsy associated with a loss-of-function mutation in Naᵥ1.1 includes Dravet Syndrome (DS) (also known as severe myoclonic epilepsy of infancy or SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; early infantile SCN1A encephalopathy; early infantile epileptic encephalopathy (EIEE); sudden unexpected death in epilepsy (SUDEP); malignant migrating partial seizures of infancy.

In some embodiments, the disease or condition is associated with a haploinsufficiency of the SCN1A gene. Exemplary diseases or conditions associated with a haploinsufficiency of the SCN1A gene include, but are not limited to, Dravet Syndrome (DS) (also known as SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); or malignant migrating partial seizures of infancy. In some cases, the disease or condition is Dravet Syndrome (DS) (also known as SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); or malignant migrating partial seizures of infancy. In some cases, the disease or condition is Dravet Syndrome (DS).

The term “epilepsy,” as used herein, refers to a group of neurological disorders characterized by recurrent epileptic seizures. “Epileptic seizures,” as used herein, refer to episodes that may vary from brief and nearly undetectable periods to long periods of vigorous shaking. Exemplary types of seizure include, but are not limited to, convulsive, non-convulsive, focal, and generalized seizures. Exemplary types of generalized seizures include, but are not limited to, tonic-clonic, tonic, clonic, myoclonic, absence, and atonic seizures.

In some embodiments, the disease or condition is characterized by a gain-of-function mutation in Naᵥ1.1. Exemplary diseases or conditions associated with a gain-of-function mutation in Na_{V}1.1 include, but are not limited to, migraine. In some instances, the disease or condition characterized by a gain-of-function mutation in Naᵥ1.1 is migraine. In some embodiments, the migraine is migraine, familial hemiplegic, 3.

In some embodiments, the method is a method of decreasing the expression of the Na_{V}1.1 protein by cells of a subject having a NIE containing pre-mRNA encoding the Na_{V}1.1 protein, and wherein the human subject has a gain-of-function mutation in Naᵥ1.1. In such an embodiment, the human subject has an allele from which the Na_{V}1.1 protein is produced in an elevated amount or an allele encoding a mutant SCN1A that induces increased activity of Na_{V}1.1 in the cell. In some embodiments, the increased activity of Na_{V}1.1 is characterized by a prolonged or near persistent sodium current mediated by the mutant Naᵥ1.1 channel, a slowing of fast inactivation, a positive shift in steady-state inactivation, higher channel availability during repetitive stimulation, increased non-inactivated depolarization-induced persistent sodium currents, delayed entry into inactivation, accelerated recovery from fast inactivation, and/or rescue of folding defects by incubation at lower temperature or co-expression of interacting proteins. In any of these embodiments, the antisense oligomer binds to a targeted portion of the NIE containing pre-mRNA transcribed from the second allele, thereby inhibiting or blocking exon skipping of the pseudo-exon from the pre-mRNA, and causing a decrease in the level of mature mRNA encoding functional Na_{V}1.1 protein, and a decrease in the expression of the Na_{V}1.1 protein in the cells of the human subject.

### Compositions

In some embodiments, the ASO provided herein comprises a sequence with at least 83%, 88%, 94% or 100% sequence identity to any one of SEQ ID NOs: 21-67, 210-256 or 304-1099. In some embodiments, the ASO consists of a sequence with at least 83%, 88%, 94% or 100% sequence identity to any one of SEQ ID NOs: 21-67, 210-256 or 304-1099. In some embodiments, the ASO comprises a sequence with at least 83%, 88%, 94% or 100% sequence identity to any one of the sequences listed in listed in Tables 4A, 4B, 5A, 5B, 6A, 6B, 7, 8A, and 8B. In some embodiments, the ASO consists of a sequence with at least 83%, 88%, 94% or 100% sequence identity to any one of the sequences listed in listed in Tables 4A, 4B, 5A, 5B, 6A, 6B, 7, 8A, and 8B.

In some embodiments, the ASO comprises a sequence with at least 60%, 65%, 66%, 67%, 68%, 69%, 70%, 71%, 72%, 73%, 74%, 75%, 76%, 77%, 78%, 79%, 80%, 81%, 82%, 83%, 884%, 85%, 86%, 87%, 88%, 89%, 90%, 91%, 92%, 93%, 94%, 95%, 96%, 97%, 98%, 99%, 99.5%, 99.8%, 99.9%, or 100% sequence identity to any one of SEQ ID NOs: 21-67, 210-256 or 304-1099. In some embodiments, the ASO consists of a sequence with at least 60%, 65%, 66%, 67%, 68%, 69%, 70%, 71%, 72%, 73%, 74%, 75%, 76%, 77%, 78%, 79%, 80%, 81%, 82%, 83%, 884%, 85%, 86%, 87%, 88%, 89%, 90%, 91%, 92%, 93%, 94%, 95%, 96%, 97%, 98%, 99%, 99.5%, 99.8%, 99.9%, or 100% sequence identity to any one of SEQ ID NOs: 21-67, 210-256 or 304-1099. In some embodiments, the ASO comprises a sequence with at least 60%, 65%, 66%, 67%, 68%, 69%, 70%, 71%, 72%, 73%, 74%, 75%, 76%, 77%, 78%, 79%, 80%, 81%, 82%, 83%, 884%, 85%, 86%, 87%, 88%, 89%, 90%, 91%, 92%, 93%, 94%, 95%, 96%, 97%, 98%, 99%, 99.5%, 99.8%, 99.9%, or 100% sequence identity to any one of the sequences listed in listed in Tables 4A, 4B, 5A, 5B, 6A, 6B, 7, 8A, and 8B. In some embodiments, the ASO consists of a sequence with at least 60%, 65%, 66%, 67%, 68%, 69%, 70%, 71%, 72%, 73%, 74%, 75%, 76%, 77%, 78%, 79%, 80%, 81%, 82%, 83%, 884%, 85%, 86%, 87%, 88%, 89%, 90%, 91%, 92%, 93%, 94%, 95%, 96%, 97%, 98%, 99%, 99.5%, 99.8%, 99.9%, or 100% sequence identity to any one of the sequences listed in listed in Tables 4A, 4B, 5A, 5B, 6A, 6B, 7, 8A, and 8B.

In some embodiments, the ASO as described herein comprises at least one modified sugar moiety.

In some embodiments, the ASO as described herein comprises T-methoxyethyl sugar moiety. In some embodiments, the T-methoxyethyl sugar moiety is a T-2′-methoxyethyl sugar moiety. In some embodiments, the ASO as described herein comprises a 2′-O-methoxyethyl moiety. In some embodiments, the ASO as described herein comprises a thymidine comprising a 2′-O-methoxyethyl moiety. In some embodiments, each nucleobase of the ASO as described herein comprises a 2′-O-methoxyethyl moiety.

In some embodiments, the ASO as described herein consists of from 8 to 50 nucleobases. In some embodiments, the ASO as described herein consists of from 16 to 20 nucleobases. In some embodiments, the ASO as described herein consists of from 12 to 20 nucleobases. In some embodiments, the ASO as described herein consists of from 8 to 20 nucleobases.

In some embodiments, the ASO as described herein consists of from 5 to 100, from 6 to 100, from 7 to 100, from 8 to 100, from 9 to 100, from 10 to 100, from 11 to 100, from 12 to 100, from 13 to 100, from 14 to 100, from 15 to 100, from 16 to 100, from 17 to 100, from 18 to 100, from 19 to 100, from 20 to 100, from 21 to 100, from 22 to 100, from 23 to 100, from 24 to 100, from 25 to 100, from 30 to 100, from 35 to 100, from 40 to 100, from 45 to 100, from 50 to 100, from 55 to 100, from 60 to 100, from 65 to 100, from 70 to 100, from 75 to 100, from 80 to 100, from 85 to 100, or from 90 to 100 nucleobases. In some embodiments, the ASO as described herein consists of from 5 to 100, 5 to 95, 5 to 90, 5 to 85, 5 to 80, 5 to 75, 5 to 70, 5 to 65, 5 to 60, 5 to 55, 5 to 50, 5 to 45, 5 to 40, 5 to 35, 5 to 30, 5 to 25, 5 to 20, 5 to 15, or 5 to 10 nucleobases. In some embodiments, the ASO as described herein consists of from 8 to 50, from 8 to 45, from 8 to 40, from 8 to 35, from 8 to 30, from 8 to 29, from 8 to 28, from 8 to 27, from 8 to 26, from 8 to 25, from 8 to 24, from 8 to 23, from 8 to 22, from 8 to 21, from 8 to 20, from 8 to 19, from 8 to 18, from 8 to 17, or from 8 to 16 nucleobases. In some embodiments, the ASO as described herein consists of from 9 to 20, from 10 to 20, from 11 to 20, from 12 to 20, from 13 to 20, from 14 to 20, from 15 to 20, from 16 to 20, from 17 to 20, or from 18 to 20 nucleobases. In some embodiments, the ASO as described herein consists of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleobases.

In some embodiments, the ASO as described herein comprises a 5′-methylcytosine (5′-MeC). In some embodiments, each cytosine of the ASO as described herein is a 5′-methylcytosine (5′-MeC).

In some embodiments, the ASO as described herein comprises a phosphorothioate linkage. In some embodiments, each internucleoside linkage of the ASO as described herein is a phosphorothioate linkage.

In some embodiments, the ASO as described herein comprises a locked nucleic acid (LNA).

In some embodiments, the ASO as described herein comprises least 1, 2, 3, 4, 5, 6, 7, 8, 9, 10, 11, 12, 13, 14, 15, 16, 17, 18, 19, or 20 LNAs. In some embodiments, the ASO as described herein comprises 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2 LNAs. In some embodiments, the ASO as described herein comprises 1, 2, 3, 4, 5, 6, 7, 8, 9, 10, 11, 12, 13, 14, 15, 16, 17, 18, 19, or 20 LNAs.

In some embodiments, 5′ end nucleotide of the ASO as described herein is an LNA. In some embodiments, 3′ end nucleotide of the ASO as described herein is an LNA. In some embodiments, 5′ and 3′ end nucleotides of the ASO as described herein are LNAs.

### ASO (Antisense Oligomer)

Provided herein is a composition comprising a compound that is an antisense oligomer that induces exon skipping by binding to a targeted portion of a SCN1A NIE containing pre-mRNA. As used herein, the terms “ASO” and “antisense oligomer” are used interchangeably and refer to an oligomer such as a polynucleotide, comprising nucleobases that hybridizes to a target nucleic acid (e.g., a SCN1A NIE containing pre-mRNA) sequence by Watson-Crick base pairing or wobble base pairing (G-U). The ASO may have exact sequence complementary to the target sequence or near complementarity (e.g., sufficient complementarity to bind the target sequence and enhancing splicing at a splice site). ASOs are designed so that they bind (hybridize) to a target nucleic acid (e.g., a targeted portion of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the pre-mRNA transcript or a sufficiently similar nucleic acid sequence in other locations in the genome or cellular pre-mRNA or transcriptome, such that the likelihood the ASO will bind other sites and cause “off-target” effects is limited. Any antisense oligomers known in the art, for example in PCT Application No. PCT/US2014/054151, published as WO 2015/035091, titled “Reducing Nonsense-Mediated mRNA Decay,” incorporated by reference herein, can be used to practice the methods described herein.

In some embodiments, ASOs “specifically hybridize” to or are “specific” to a target nucleic acid or a targeted portion of a NIE containing pre-mRNA. Typically, such hybridization occurs with a Tₘ substantially greater than 37° C., preferably at least 50° C., and typically between 60° C., to approximately 90° C. Such hybridization preferably corresponds to stringent hybridization conditions. At a given ionic strength and pH, the Tₘ is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an antisense oligomer (ASO) need not be 100% complementary to that of its target nucleic acid to hybridize. In some embodiments, ASOs can comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining non-complementary nucleobases may be clustered together or interspersed with complementary nucleobases and need not be contiguous to each other or to complementary nucleobases. Percent complementarity of an ASO with a region of a target nucleic acid can be determined routinely using BLAST programs (basic local alignment search tools) and PowerBLAST programs known in the art (Altschul, et al., J. Mol. Biol., 1990, 215, 403-410; Zhang and Madden, Genome Res., 1997, 7, 649-656, of which entire content is incorporated herein by reference).

An ASO need not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In some embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

In some cases, an ASO described herein is all-P-ambo-2′-O-(2-methoxyethyl)-P-thioadenylyl-(3′→5′)-2′-O-(2-methoxyethyl)-P-thioguanylyl-(3′→5′)-2′-O-(2-methoxyethyl)-5-methyl-P-thiouridylyl-(3′→5′)-2′-O-(2-methoxyethyl)-5-methyl-P-thiouridylyl-(3′→5′)-2′-O-(2-methoxyethyl)-P-thioguanylyl-(3′→5′)-2′-O-(2-methoxyethyl)-P-thioguanylyl-(3′→5′)-2′-O-(2-methoxyethyl)-P-thioadenylyl-(3′→5′)-2′-O-(2-methoxyethyl)-P-thioguanylyl-(3′→5′)-2′-O-(2-methoxyethyl)-5-methyl-P-thiocytidylyl-(3′→5′)-2′-O-(2-methoxyethyl)-P-thioadenylyl-(3′→5′)-2′-O-(2-methoxyethyl)-P-thioadenylyl-(3′→5′)-2′-O-(2-methoxyethyl)-P-thioguanylyl-(3′→5′)-2′-O-(2-methoxyethyl)-P-thioadenylyl-(3′→5′)-2′-O-(2-methoxyethyl)-5-methyl-P-thiouridylyl-(3′→5′)-2′-O-(2-methoxyethyl)-5-methyl-P-thiouridylyl-(3′→5′)-2′-O-(2-methoxyethyl)-P-thioadenylyl-(3′→5′)-2′-O-(2-methoxyethyl)-5-methyl-P-thiouridylyl-(3′→5′)-2′-O-(2-methoxyethyl)-5-methylcytidine (SEQ ID NO: 1100), or a salt thereof.

In some cases, an ASO described herein is a sodium salt of all-P-ambo-2′-O-(2-methoxyethyl)-P-thioadenylyl-(3′→5′)-2′-O-(2-methoxyethyl)-P-thioguanylyl-(3′→5′)-2′-O-(2-methoxyethyl)-5-methyl-P-thiouridylyl-(3′→5′)-2′-O-(2-methoxyethyl)-5-methyl-P-thiouridylyl-(3′→5′)-2′-O-(2-methoxyethyl)-P-thioguanylyl-(3′→5′)-2′-O-(2-methoxyethyl)-P-thioguanylyl-(3′→5′)-2′-O-(2-methoxyethyl)-P-thioadenylyl-(3′→5′)-2′-O-(2-methoxyethyl)-P-thioguanylyl-(3′→5′)-2′-O-(2-methoxyethyl)-5-methyl-P-thiocytidylyl-(3′→5′)-2′-O-(2-methoxyethyl)-P-thioadenylyl-(3′→5′)-2′-O-(2-methoxyethyl)-P-thioadenylyl-(3′→5′)-2′-O-(2-methoxyethyl)-P-thioguanylyl-(3′→5′)-2′-O-(2-methoxyethyl)-P-thioadenylyl-(3′→5′)-2′-O-(2-methoxyethyl)-5-methyl-P-thiouridylyl-(3′→5′)-2′-O-(2-methoxyethyl)-5-methyl-P-thiouridylyl-(3′→5′)-2′-O-(2-methoxyethyl)-P-thioadenylyl-(3′→5′)-2′-O-(2-methoxyethyl)-5-methyl-P-thiouridylyl-(3′→5′)-2′-O-(2-methoxyethyl)-5-methylcytidine (SEQ ID NO: 1100).

In some cases, a compound (e.g., an ASO) described herein is compound (I) having the structure depicted in formula (I) (free acid) (SEQ ID NO: 1100):

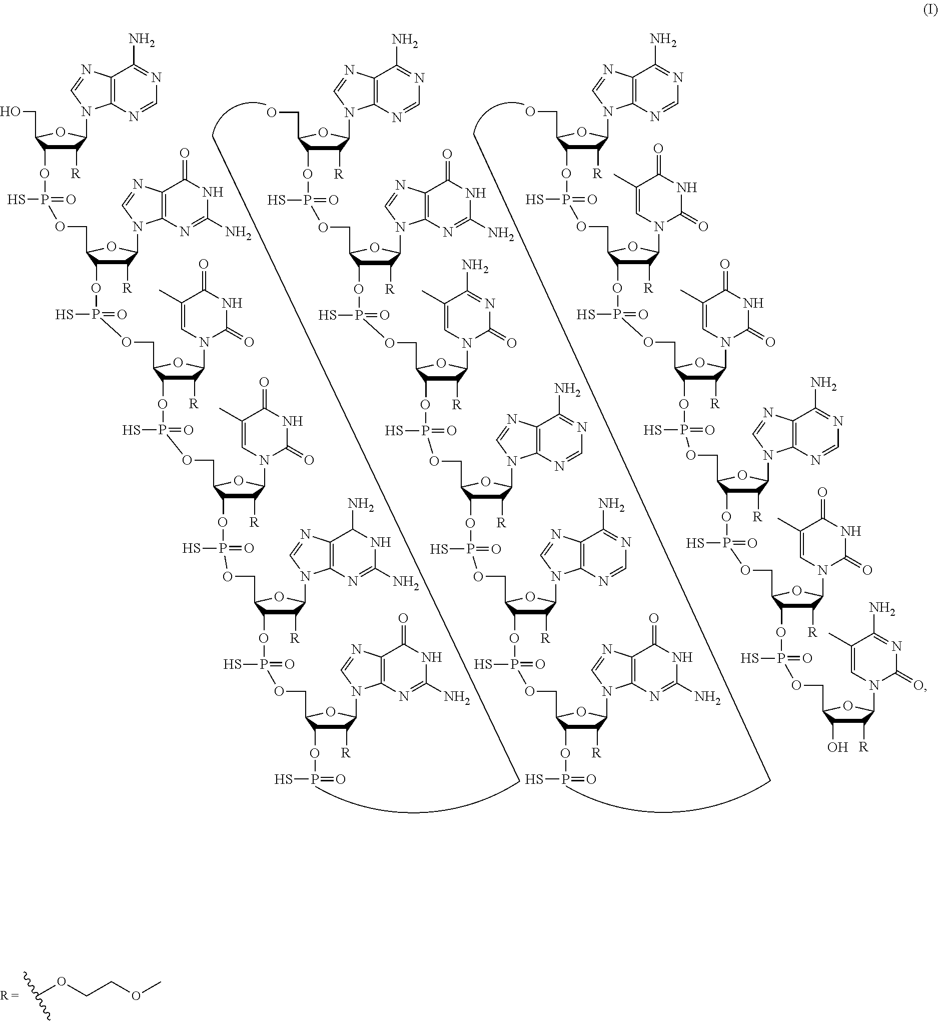

or a salt thereof.

In some cases, a compound (e.g., an ASO) described herein is compound (II) having the structure depicted in formula (II) (sodium salt) (SEQ ID NO: 1100):

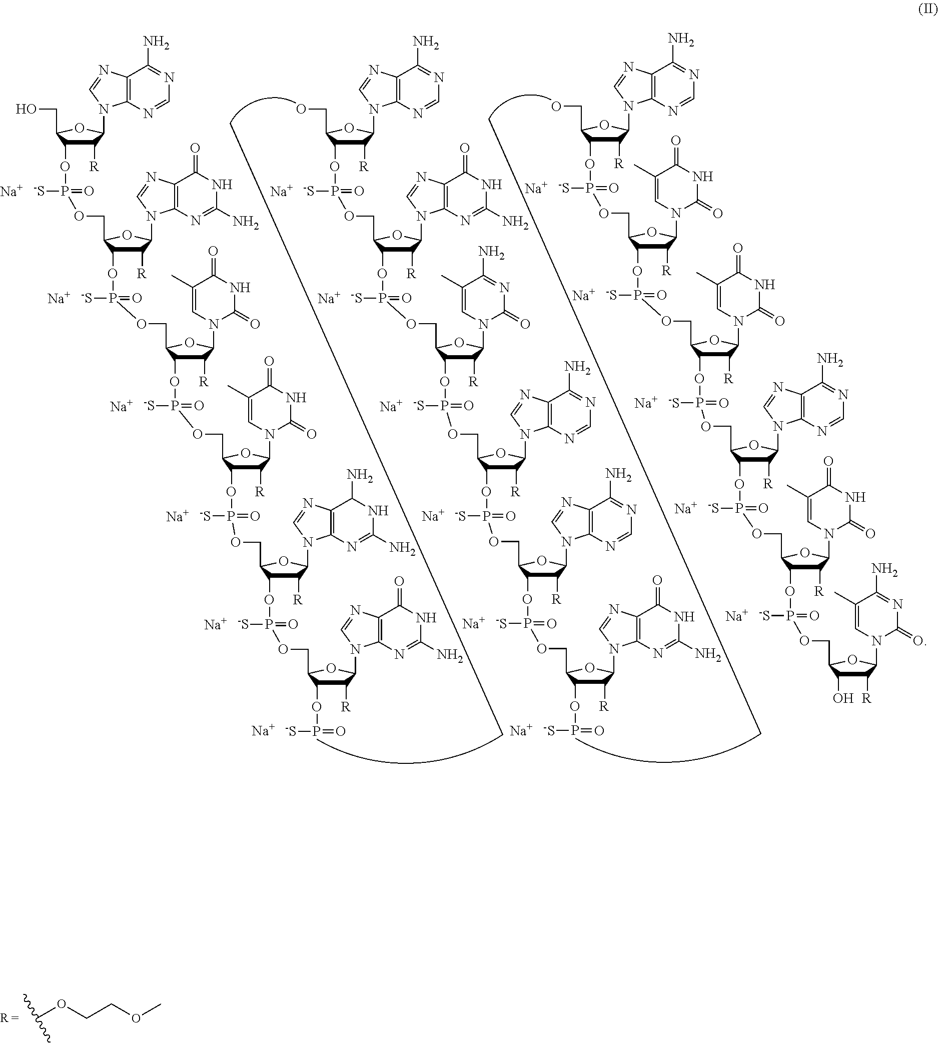

In any of the structural formulae (graphic representation of a chemical compound) presented herein, where two curved lines and a straight line in between them are used to connect a phosphorus atom (“P”) and an oxygen atom (“O”), the two curved line and the straight line therebetween should be seen as a single integral segment, representing the covalent bond between the phosphorus atom and the oxygen atom, which is part of a backbone linkage (e.g., phosphodiester linkage or phosphorothioate linkage) between two neighboring nucleotides. Any of the vertices (corners) in any of those structural formulae where a curved line and a straight line join does not represent carbon atom or presence of —CH2- at the relevant location in the compound the structural formula represents.

The compounds described herein (e.g., compound (I) or a salt thereof, or compound (II)) comprise nucleobases that are complementary to nucleobases present in a targeted portion of a NIE containing pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art and can be found, for example, in U.S. Pat. No. 8,258,109 B2, U.S. Pat. No. 5,656,612, U.S. Patent Publication No. 2012/0190728, and Dias and Stein, Mol. Cancer Ther. 2002, 347-355, herein incorporated by reference in their entirety.

One or more nucleobases of an ASO (e.g., compound (I) or a salt thereof, or compound (II)) may be any naturally occurring, unmodified nucleobase such as adenine, guanine, cytosine, thymine and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5,6-dihydrouracil, 5-methylcytosine, and 5-hydroxymethoylcytosine.

The compounds described herein (e.g., compound (I) or a salt thereof, or compound (II)) also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligomer linkages of the ASOs described herein may include (but are not limited to) phosphorothioate, phosphorodithioate, phosphoroselenoate, phosphorodiselenoate, phosphoroanilothioate, phosphoraniladate, phosphoramidate, and the like. See, e.g., LaPlanche, et al., Nucleic Acids Res. 14:9081 (1986); Stec, et al., J. Am. Chem. Soc. 106:6077 (1984), Stein, et al., Nucleic Acids Res. 16:3209 (1988), Zon, et al., Anti-Cancer Drug Design 6:539 (1991); Zon, et al., Oligonucleotides and Analogues: A Practical Approach, pp. 87-108 (F. Eckstein, Ed., Oxford University Press, Oxford England (1991)); Stec, et al., U.S. Pat. No. 5,151,510; Uhlmann and Peyman, Chemical Reviews 90:543 (1990), of which entire content is incorporated herein by reference. In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphorothioate linkage. In some embodiments, the backbone modification is a phosphoramidate linkage.

In some embodiments, the stereochemistry at each of the phosphorus internucleotide linkages of the ASO backbone is random. In some embodiments, the stereochemistry at each of the phosphorus internucleotide linkages of the ASO backbone is controlled and is not random. For example, U.S. Pat. App. Pub. No. 2014/0194610, “Methods for the Synthesis of Functionalized Nucleic Acids,” incorporated herein by reference, describes methods for independently selecting the handedness of chirality at each phosphorous atom in a nucleic acid oligomer. In some embodiments, an ASO used in the methods of the invention, including, but not limited to, any of the ASOs set forth herein in Tables 5 and 6, comprises an ASO having phosphorus internucleotide linkages that are not random. In some embodiments, a composition used in the methods of the invention comprises a pure diastereomeric ASO. In some embodiments, a composition used in the methods of the invention comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some embodiments, the ASO (e.g., compound (I) or a salt thereof, or compound (II)) has a nonrandom mixture of Rp and Sp configurations at its phosphorus internucleotide linkages. For example, it has been suggested that a mix of Rp and Sp is required in antisense oligonucleotides or antisense oligomers to achieve a balance between good activity and nuclease stability (Wan, et al., 2014, “Synthesis, biophysical properties and biological activity of second-generation antisense oligonucleotides containing chiral phosphorothioate linkages,” Nucleic Acids Research 42 (22): 13456-13468, incorporated herein by reference). In some embodiments, an ASO used in the methods of the invention, including, but not limited to, any of the ASOs set forth herein in SEQ ID NOs: 21-114, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In some embodiments, an ASO used in the methods of the invention, including, but not limited to, any of the ASOs set forth herein in SEQ ID NOs: 21-114, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In some embodiments, an ASO used in the methods of the invention, including, but not limited to, any of the ASOs set forth herein in SEQ ID NOs: 21-114, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In some embodiments, an ASO used in the methods of the invention, including, but not limited to, any of the ASOs set forth herein in SEQ ID NOs: 21-114, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

In some embodiments, an ASO used in the methods of the invention, including, but not limited to, any of the ASOs set forth herein in SEQ ID NOs: 21-67, 210-256 or 304-1099, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In some embodiments, an ASO used in the methods of the invention, including, but not limited to, any of the ASOs set forth herein in SEQ ID NOs: 21-67, 210-256 or 304-1099, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In some embodiments, an ASO used in the methods of the invention, including, but not limited to, any of the ASOs set forth herein in SEQ ID NOs: 21-67, 210-256 or 304-1099, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In some embodiments, an ASO used in the methods of the invention, including, but not limited to, any of the ASOs set forth herein in SEQ ID NOs: 21-67, 210-256 or 304-1099, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

In some embodiments, an ASO used in the methods of the invention, including, but not limited to, any of the ASOs set forth herein in any one of the sequences listed in listed in Tables 4A, 4B, 5A, 5B, 6A, 6B, 7, 8A, and 8B, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In some embodiments, an ASO used in the methods of the invention, including, but not limited to, any of the ASOs set forth herein in any one of the sequences listed in listed in Tables 4A, 4B, 5A, 5B, 6A, 6B, 7, 8A, and 8B, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In some embodiments, an ASO used in the methods of the invention, including, but not limited to, any of the ASOs set forth herein in any one of the sequences listed in listed in Tables 4A, 4B, 5A, 5B, 6A, 6B, 7, 8A, and 8B, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In some embodiments, an ASO used in the methods of the invention, including, but not limited to, any of the ASOs set forth herein in any one of the sequences listed in listed in Tables 4A, 4B, 5A, 5B, 6A, 6B, 7, 8A, and 8B, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

In some embodiments, an ASO used in the methods of the invention, having the structure of formula (I) or (II), comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In some embodiments, an ASO used in the methods of the invention, having the structure of formula (I) or (II), comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

Any of the compounds (e.g., ASOs) described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl (2′MOE), 2′-O-aminoethyl, 2′F, 2′-N-methyl-acetamide (2′-NMA); N3′→P5′ phosphoramidate, 2′ dimethylaminooxyethoxy, 2′ dimethylaminoethoxyethoxy, 2′-guanidinidium, 2′-O-guanidinium ethyl, carbamate modified sugars, and bicyclic modified sugars. In some embodiments, the sugar moiety modification is selected from 2′-O-Me, 2′F, 2′MOE, and 2′-NMA. As used herein, “2′-NMA” means a —O—CH2-C(═O)—NH—CH3 group in place of the 2′-OH group of a ribosyl sugar moiety. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuransyl or 2′ deoxyribofuransyl modification. In some embodiments, the sugar moiety comprises 2′4′-constrained 2′O-methyloxyethyl (cMOE) modifications. In some embodiments, the sugar moiety comprises cEt 2′,4′ constrained 2′-O ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art and described in the literature, e.g., by Jarver, et al., 2014, Nucleic Acid Therapeutics 24 (1): 37-47, incorporated by reference for this purpose herein. “A Chemical View of Oligonucleotides for Exon Skipping and Related Drug Applications,” Nucleic Acid Therapeutics 24 (1): 37-47, incorporated by reference for this purpose herein.

In some embodiments, each monomer of the ASO is modified in the same way, for example each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some examples, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modifications. In some embodiments, the ASO comprises one or more sugar moiety modification. In some embodiments, the ASO comprises one or more backbone modifications and one or more sugar moiety modifications. In some embodiments, the ASO comprises a 2′MOE modification and a phosphorothioate backbone. In some embodiments, the ASO comprises a phosphorodiamidate morpholino (PMO). In some embodiments, the ASO comprises a peptide nucleic acid (PNA). Any of the ASOs or any component of an ASO (e.g., a nucleobase, sugar moiety, backbone) described herein may be modified in order to achieve desired properties or activities of the ASO or reduce undesired properties or activities of the ASO. For example, an ASO or one or more components of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and/or modulate the half-life of the ASO.

In some embodiments, the ASOs are comprised of 2′-O-(2-methoxyethyl) (MOE) phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary, et al., J Pharmacol Exp Ther. 2001; 296 (3): 890-7; Geary, et al., J Pharmacol Exp Ther. 2001; 296 (3): 898-904, of which entire content is incorporated herein by reference.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is 5′ end and the left-hand direction of single or double-stranded nucleic acid sequences is referred to as 5′ direction. Similarly, the right-hand end or direction of a nucleic acid sequence (single or double stranded) is 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, 5′ direction or end of an mRNA is where the initiation or start codon is located, while 3′ end or direction is where the termination codon is located. In some aspects, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

In some embodiments, the ASOs are complementary to (and bind to) a targeted portion of a SCN1A NIE containing pre-mRNA that is downstream (in 3′ direction) of 5′ splice site (or 3′ end of the NIE) of the included exon in a SCN1A NIE containing pre-mRNA (e.g., the direction designated by positive numbers relative to 5′ splice site). In some embodiments, the ASOs are complementary to a targeted portion of the SCN1A NIE containing pre-mRNA that is within the region about +1 to about +500 relative to 5′ splice site (or 3′ end) of the included exon. In some embodiments, the ASOs may be complementary to a targeted portion of a SCN1A NIE containing pre-mRNA that is within the region between nucleotides +6 and +496 relative to the 5′ splice site (or 3′ end) of the included exon. In some aspects, the ASOs are complementary to a targeted portion that is within the region about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20 relative to 5′ splice site (or 3′ end) of the included exon. In some aspects, the ASOs are complementary to a targeted portion that is within the region from about +1 to about +100, from about +100 to about +200, from about +200 to about +300, from about +300 to about +400, or from about +400 to about +500 relative to 5′ splice site (or 3′ end) of the included exon.

In some embodiments, the ASOs are complementary to (and bind to) a targeted portion of a SCN1A NIE containing pre-mRNA that is upstream (in 5′ direction) of 5′ splice site (or 3′ end) of the included exon in a SCN1A NIE containing pre-mRNA (e.g., the direction designated by negative numbers relative to 5′ splice site). In some embodiments, the ASOs are complementary to a targeted portion of the SCN1A NIE containing pre-mRNA that is within the region about −4 to about −270 relative to 5′ splice site (or 3′ end) of the included exon. In some embodiments, the ASOs may be complementary to a targeted portion of a SCN1A NIE containing pre-mRNA that is within the region between nucleotides −1 and −264 relative to 5′ splice site (or 3′ end) of the included exon. In some aspects, the ASOs are complementary to a targeted portion that is within the region about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 5′ splice site (or 3′ end) of the included exon. In some aspects, the ASOs are complementary to a targeted portion that is within the region from about −1 to about −50, from about −50 to about −100, from about −100 to about −150, from about −150 to about −200, or from about −200 to about −250 relative to 5′ splice site (or 3′ end) of the included exon.

In some embodiments, the ASOs are complementary to a targeted region of a SCN1A NIE containing pre-mRNA that is upstream (in 5′ direction) of 3′ splice site (or 5′ end) of the included exon in a SCN1A NIE containing pre-mRNA (e.g., in the direction designated by negative numbers). In some embodiments, the ASOs are complementary to a targeted portion of the SCN1A NIE containing pre-mRNA that is within the region about −1 to about −500 relative to the 3′ splice site (or 5′ end) of the included exon. In some embodiments, the ASOs are complementary to a targeted portion of the SCN1A NIE containing pre-mRNA that is within the region −1 to −496 relative to 3′ splice site of the included exon. In some aspects, the ASOs are complementary to a targeted portion that is within the region about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, or about −1 to about −30 relative to 3′ splice site of the included exon. In some aspects, the ASOs are complementary to a targeted portion that is within the region from about −1 to about −100, from about −100 to about −200, from about −200 to about −300, from about −300 to about −400, or from about −400 to about −500 relative to 3′ splice site of the included exon.

In some embodiments, the ASOs are complementary to a targeted region of a SCN1A NIE containing pre-mRNA that is downstream (in 3′ direction) of 3′ splice site (5′ end) of the included exon in a SCN1A NIE containing pre-mRNA (e.g., in the direction designated by positive numbers). In some embodiments, the ASOs are complementary to a targeted portion of the SCN1A NIE containing pre-mRNA that is within the region of about +1 to about +100 relative to 3′ splice site of the included exon. In some aspects, the ASOs are complementary to a targeted portion that is within the region about +1 to about +90, about +1 to about +80, about +1 to about +70, about +1 to about +60, about +1 to about +50, about +1 to about +40, about +1 to about +30, about +1 to about +20, or about +1 to about +10 relative to 3′ splice site of the included exon.

In some embodiments, the targeted portion of the SCN1A NIE containing pre-mRNA is within the region +100 relative to 5′ splice site (3′ end) of the included exon to −100 relative to 3′ splice site (5′ end) of the included exon. In some embodiments, the targeted portion of the SCN1A NIE containing pre-mRNA is within the NIE. In some embodiments, the targeted portion of the SCN1A NIE containing pre-mRNA comprises a pseudo-exon and intron boundary.

The ASOs may be of any length suitable for specific binding and effective enhancement of splicing. In some embodiments, the ASOs consist of 8 to 50 nucleobases. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleobases in length. In some embodiments, the ASOs consist of more than 50 nucleobases. In some embodiments, the ASO is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 13 to 50 nucleobases, 13 to 40 nucleobases, 13 to 35 nucleobases, 13 to 30 nucleobases, 13 to 25 nucleobases, 13 to 20 nucleobases, 14 to 50 nucleobases, 14 to 40 nucleobases, 14 to 35 nucleobases, 14 to 30 nucleobases, 14 to 25 nucleobases, 14 to 20 nucleobases, 15 to 50 nucleobases, 15 to 40 nucleobases, 15 to 35 nucleobases, 15 to 30 nucleobases, 15 to 25 nucleobases, 15 to 20 nucleobases, 20 to 50 nucleobases, 20 to 40 nucleobases, 20 to 35 nucleobases, 20 to 30 nucleobases, 20 to 25 nucleobases, 25 to 50 nucleobases, 25 to 40 nucleobases, 25 to 35 nucleobases, or 25 to 30 nucleobases in length. In some embodiments, the ASOs are 18 nucleotides in length. In some embodiments, the ASOs are 15 nucleotides in length. In some embodiments, the ASOs are 25 nucleotides in length.

In some embodiments, two or more ASOs with different chemistries but complementary to the same targeted portion of the NIE containing pre-mRNA are used. In some embodiments, two or more ASOs that are complementary to different targeted portions of the NIE containing pre-mRNA are used.

In some embodiments, the antisense oligomers of the present disclosure (e.g., compound (I) or a salt thereof, or compound (II)) are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine, or a polyethylene glycol chain, or adamantane acetic acid. Oligonucleotides comprising lipophilic moieties and preparation methods have been described in the published literature. In some embodiments, the antisense oligomer is conjugated with a moiety including, but not limited to, an abasic nucleotide, a polyether, a polyamine, a polyamide, a peptide, a carbohydrate, e.g., N-acetylgalactosamine (GalNAc), N-Ac-Glucosamine (GluNAc), or mannose (e.g., mannose-6-phosphate), a lipid, or a polyhydrocarbon compound. Conjugates can be linked to one or more of any nucleotides comprising the antisense oligomer at any of several positions on the sugar, base or phosphate group, as understood in the art and described in the literature, e.g., using a linker. Linkers can include a bivalent or trivalent branched linker. In some embodiments, the conjugate is attached to the 3′ end of the antisense oligomer. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, the nucleic acid to be targeted by an ASO is a SCN1A NIE containing pre-mRNA expressed in a cell, such as a eukaryotic cell. In some embodiments, the term “cell” may refer to a population of cells. In some embodiments, the cell is in a subject. In some embodiments, the cell is isolated from a subject. In some embodiments, the cell is ex vivo. In some embodiments, the cell is a condition or disease-relevant cell or a cell line. In some embodiments, the cell is in vitro (e.g., in cell culture).

In some embodiments, the compound is the salt of a nucleotide. In some embodiments, the compound is the salt of a nucleotide, fully phosphorothioate-linked oligonucleotide. In some embodiments, the compound is the salt of a nucleotide in which the salt binds to the phosphate-link. In some embodiments, the compound is the salt of a nucleotide, fully phosphorothioate-linked oligonucleotide in which the salt binds to the phosphate-link. In some embodiments, the compound is the sodium salt of a nucleotide. In some embodiments, the compound is the sodium salt of a nucleotide, fully phosphorothioate-linked oligonucleotide. In some embodiments, the compound is the sodium salt of a nucleotide in which the sodium salt binds to the phosphate-link. In some embodiments, the compound is the sodium salt of a nucleotide, fully phosphorothioate-linked oligonucleotide in which the sodium salt binds to the phosphate-link. In some embodiments, the compound is the potassium salt of a nucleotide. In some embodiments, the compound is the potassium salt of a nucleotide, fully phosphorothioate-linked oligonucleotide. In some embodiments, the compound is the potassium salt of a nucleotide in which the potassium salt binds to the phosphate-link. In some embodiments, the compound is the potassium salt of a nucleotide, fully phosphorothioate-linked oligonucleotide in which the potassium salt binds to the phosphate-link. In some cases, the compound is compound (I) or a salt thereof. In some cases, the compound is compound (II).

In some embodiments, the compound is the monosodium salt of a 2-nucleotide (2-mer). In some embodiments, the compound is the disodium salt of a 3-nucleotide (3-mer). In some embodiments, the compound is the trisodium salt of a 4-nucleotide (4-mer). In some embodiments, the compound is the tetrasodium salt of a 5-nucleotide (5-mer). In some embodiments, the compound is the pentasodium salt of a 6-nucleotide (6-mer). In some embodiments, the compound is the hexasodium salt of a 7-nucleotide (7-mer). In some embodiments, the compound is the heptasodium salt of an 8-nucleotide (8-mer). In some embodiments, the compound is the octasodium salt of a 9-nucleotide (9-mer). In some embodiments, the compound is the nonasodium salt of a 10-nucleotide (10-mer). In some embodiments, the compound is the decasodium salt of a 11-nucleotide (11-mer). In some embodiments, the compound is the undecasodium salt of a 12-nucleotide (12-mer). In some embodiments, the compound is the dodecasodium salt of a 13-nucleotide (13-mer). In some embodiments, the compound is the tridecasodium salt of a 14-nucleotide (14-mer). In some embodiments, the compound is the tetradecasodium salt of a 15-nucleotide (15-mer). In some embodiments, the compound is the pentadecasodium salt of a 16-nucleotide (16-mer). In some embodiments, the compound is the hexadecasodium salt of a 17-nucleotide (17-mer). In some embodiments, the compound is the heptadecasodium salt of an 18-nucleotide (18-mer). In some embodiments, the compound is the octadecasodium salt of a 19-nucleotide (19-mer). In some embodiments, the compound is the nonadecasodium salt of a 20-nucleotide (20-mer). In some embodiments, the compound is the icosasodium salt of a 21-nucleotide (21-mer). In some embodiments, the compound is the henicosasodium salt of a 22-nucleotide (22-mer). In some embodiments, the compound is the docosasodium salt of a 23-nucleotide (23-mer). In some embodiments, the compound is the tricosasodium salt of a 24-nucleotide (24-mer). In some embodiments, the compound is the tetracosasodium salt of a 25-nucleotide (25-mer). In some embodiments, the compound is the pentacosasodium salt of a 26-nucleotide (26-mer). In some embodiments, the compound is the hexacosasodium salt of a 27-nucleotide (27-mer). In some embodiments, the compound is the heptacosasodium salt of a 28-nucleotide (28-mer). In some embodiments, the compound is the octacosasodium salt of a 29-nucleotide (29-mer). In some embodiments, the compound is the nonacosasodium salt of a 30-nucleotide (30-mer). In some embodiments, the compound is the triacontasodium salt of a 31-nucleotide (31-mer). In some embodiments, the compound is the hentriacontasodium salt of a 32-nucleotide (32-mer). In some embodiments, the compound is the dotriacontasodium salt of a 33-nucleotide (33-mer). In some embodiments, the compound is the tritriacontasodium salt of a 34-nucleotide (34-mer). In some embodiments, the compound is the tetratriacontasodium salt of a 35-nucleotide (35-mer). In some embodiments, the compound is the pentatriacontasodium salt of a 36-nucleotide (36-mer). In some embodiments, the compound is the hexatriacontasodium salt of a 37-nucleotide (37-mer). In some embodiments, the compound is the heptatriacontasodium salt of a 38-nucleotide (38-mer). In some embodiments, the compound is the octatriacontasodium salt of a 39-nucleotide (39-mer). In some embodiments, the compound is the nonatriacontasodium salt of a 40-nucleotide (40-mer). In some embodiments, the compound is the tetracontasodium salt of a 41-nucleotide (41-mer). In some embodiments, the compound is the hentetracontasodium salt of a 42-nucleotide (42-mer). In some embodiments, the compound is the dotetracontasodium salt of a 43-nucleotide (43-mer). In some embodiments, the compound is the tritetracontasodium salt of a 44-nucleotide (44-mer). In some embodiments, the compound is the tetratetracontasodium salt of a 45-nucleotide (45-mer). In some embodiments, the compound is the pentatetracontasodium salt of a 46-nucleotide (46-mer). In some embodiments, the compound is the hexatetracontasodium salt of a 47-nucleotide (47-mer). In some embodiments, the compound is the heptatetracontasodium salt of a 48-nucleotide (48-mer). In some embodiments, the compound is the octatetracontasodium salt of a 49-nucleotide (49-mer). In some embodiments, the compound is the nonatetracontasodium salt of a 50-nucleotide (50-mer). In some embodiments, the compound is the pentacontasodium salt of a 51-nucleotide (51-mer).

In some embodiments, the compound is the monosodium salt of a 2-nucleotide (2-mer), fully phosphorothioate-linked oligonucleotide. In some embodiments, the compound is the disodium salt of a 3-nucleotide (3-mer), fully phosphorothioate-linked oligonucleotide. In some embodiments, the compound is the trisodium salt of a 4-nucleotide (4-mer), fully phosphorothioate-linked oligonucleotide. In some embodiments, the compound is the tetrasodium salt of a 5-nucleotide (5-mer), fully phosphorothioate-linked oligonucleotide. In some embodiments, the compound is the pentasodium salt of a 6-nucleotide (6-mer), fully phosphorothioate-linked oligonucleotide. In some embodiments, the compound is the hexasodium salt of a 7-nucleotide (7-mer), fully phosphorothioate-linked oligonucleotide. In some embodiments, the compound is the heptasodium salt of an 8-nucleotide (8-mer), fully phosphorothioate-linked oligonucleotide. In some embodiments, the compound is the octasodium salt of a 9-nucleotide (9-mer), fully phosphorothioate-linked oligonucleotide. In some embodiments, the compound is the nonasodium salt of a 10-nucleotide (10-mer), fully phosphorothioate-linked oligonucleotide. In some embodiments, the compound is the decasodium salt of a 11-nucleotide (11-mer), fully phosphorothioate-linked oligonucleotide. In some embodiments, the compound is the undecasodium salt of a 12-nucleotide (12-mer), fully phosphorothioate-linked oligonucleotide. In some embodiments, the compound is the dodecasodium salt of a 13-nucleotide (13-mer), fully phosphorothioate-linked oligonucleotide. In some embodiments, the compound is the tridecasodium salt of a 14-nucleotide (14-mer), fully phosphorothioate-linked oligonucleotide. In some embodiments, the compound is the tetradecasodium salt of a 15-nucleotide (15-mer), fully phosphorothioate-linked oligonucleotide. In some embodiments, the compound is the pentadecasodium salt of a 16-nucleotide (16-mer), fully phosphorothioate-linked oligonucleotide. In some embodiments, the compound is the hexadecasodium salt of a 17-nucleotide (17-mer), fully phosphorothioate-linked oligonucleotide. In some embodiments, the compound is the heptadecasodium salt of an 18-nucleotide (18-mer), fully phosphorothioate-linked oligonucleotide. In some embodiments, the compound is the octadecasodium salt of a 19-nucleotide (19-mer), fully phosphorothioate-linked oligonucleotide. In some embodiments, the compound is the nonadecasodium salt of a 20-nucleotide (20-mer), fully phosphorothioate-linked oligonucleotide. In some embodiments, the compound is the icosasodium salt of a 21-nucleotide (21-mer), fully phosphorothioate-linked oligonucleotide. In some embodiments, the compound is the henicosasodium salt of a 22-nucleotide (22-mer), fully phosphorothioate-linked oligonucleotide. In some embodiments, the compound is the docosasodium salt of a 23-nucleotide (23-mer), fully phosphorothioate-linked oligonucleotide. In some embodiments, the compound is the tricosasodium salt of a 24-nucleotide (24-mer), fully phosphorothioate-linked oligonucleotide. In some embodiments, the compound is the tetracosasodium salt of a 25-nucleotide (25-mer), fully phosphorothioate-linked oligonucleotide. In some embodiments, the compound is the pentacosasodium salt of a 26-nucleotide (26-mer), fully phosphorothioate-linked oligonucleotide. In some embodiments, the compound is the hexacosasodium salt of a 27-nucleotide (27-mer), fully phosphorothioate-linked oligonucleotide. In some embodiments, the compound is the heptacosasodium salt of a 28-nucleotide (28-mer), fully phosphorothioate-linked oligonucleotide. In some embodiments, the compound is the octacosasodium salt of a 29-nucleotide (29-mer), fully phosphorothioate-linked oligonucleotide. In some embodiments, the compound is the nonacosasodium salt of a 30-nucleotide (30-mer), fully phosphorothioate-linked oligonucleotide. In some embodiments, the compound is the triacontasodium salt of a 31-nucleotide (31-mer), fully phosphorothioate-linked oligonucleotide. In some embodiments, the compound is the hentriacontasodium salt of a 32-nucleotide (32-mer), fully phosphorothioate-linked oligonucleotide. In some embodiments, the compound is the dotriacontasodium salt of a 33-nucleotide (33-mer), fully phosphorothioate-linked oligonucleotide. In some embodiments, the compound is the tritriacontasodium salt of a 34-nucleotide (34-mer), fully phosphorothioate-linked oligonucleotide. In some embodiments, the compound is the tetratriacontasodium salt of a 35-nucleotide (35-mer), fully phosphorothioate-linked oligonucleotide. In some embodiments, the compound is the pentatriacontasodium salt of a 36-nucleotide (36-mer), fully phosphorothioate-linked oligonucleotide. In some embodiments, the compound is the hexatriacontasodium salt of a 37-nucleotide (37-mer), fully phosphorothioate-linked oligonucleotide. In some embodiments, the compound is the heptatriacontasodium salt of a 38-nucleotide (38-mer), fully phosphorothioate-linked oligonucleotide. In some embodiments, the compound is the octatriacontasodium salt of a 39-nucleotide (39-mer), fully phosphorothioate-linked oligonucleotide. In some embodiments, the compound is the nonatriacontasodium salt of a 40-nucleotide (40-mer), fully phosphorothioate-linked oligonucleotide. In some embodiments, the compound is the tetracontasodium salt of a 41-nucleotide (41-mer), fully phosphorothioate-linked oligonucleotide. In some embodiments, the compound is the hentetracontasodium salt of a 42-nucleotide (42-mer), fully phosphorothioate-linked oligonucleotide. In some embodiments, the compound is the dotetracontasodium salt of a 43-nucleotide (43-mer), fully phosphorothioate-linked oligonucleotide. In some embodiments, the compound is the tritetracontasodium salt of a 44-nucleotide (44-mer), fully phosphorothioate-linked oligonucleotide. In some embodiments, the compound is the tetratetracontasodium salt of a 45-nucleotide (45-mer), fully phosphorothioate-linked oligonucleotide. In some embodiments, the compound is the pentatetracontasodium salt of a 46-nucleotide (46-mer), fully phosphorothioate-linked oligonucleotide. In some embodiments, the compound is the hexatetracontasodium salt of a 47-nucleotide (47-mer), fully phosphorothioate-linked oligonucleotide. In some embodiments, the compound is the heptatetracontasodium salt of a 48-nucleotide (48-mer), fully phosphorothioate-linked oligonucleotide. In some embodiments, the compound is the octatetracontasodium salt of a 49-nucleotide (49-mer), fully phosphorothioate-linked oligonucleotide. In some embodiments, the compound is the nonatetracontasodium salt of a 50-nucleotide (50-mer), fully phosphorothioate-linked oligonucleotide. In some embodiments, the compound is the pentacontasodium salt of a 51-nucleotide (51-mer), fully phosphorothioate-linked oligonucleotide.

In some embodiments, the compound is the monopotassium salt of a 2-nucleotide (2-mer). In some embodiments, the compound is the dipotassium salt of a 3-nucleotide (3-mer). In some embodiments, the compound is the tripotassium salt of a 4-nucleotide (4-mer). In some embodiments, the compound is the tetrapotassium salt of a 5-nucleotide (5-mer). In some embodiments, the compound is the pentapotassium salt of a 6-nucleotide (6-mer). In some embodiments, the compound is the hexapotassium salt of a 7-nucleotide (7-mer). In some embodiments, the compound is the heptapotassium salt of an 8-nucleotide (8-mer). In some embodiments, the compound is the octapotassium salt of a 9-nucleotide (9-mer). In some embodiments, the compound is the nonapotassium salt of a 10-nucleotide (10-mer). In some embodiments, the compound is the decapotassium salt of a 11-nucleotide (11-mer). In some embodiments, the compound is the undecapotassium salt of a 12-nucleotide (12-mer). In some embodiments, the compound is the dodecapotassium salt of a 13-nucleotide (13-mer). In some embodiments, the compound is the tridecapotassium salt of a 14-nucleotide (14-mer). In some embodiments, the compound is the tetradecapotassium salt of a 15-nucleotide (15-mer). In some embodiments, the compound is the pentadecapotassium salt of a 16-nucleotide (16-mer). In some embodiments, the compound is the hexadecapotassium salt of a 17-nucleotide (17-mer). In some embodiments, the compound is the heptadecapotassium salt of an 18-nucleotide (18-mer). In some embodiments, the compound is the octadecapotassium salt of a 19-nucleotide (19-mer). In some embodiments, the compound is the nonadecapotassium salt of a 20-nucleotide (20-mer). In some embodiments, the compound is the icosapotassium salt of a 21-nucleotide (21-mer). In some embodiments, the compound is the henicosapotassium salt of a 22-nucleotide (22-mer). In some embodiments, the compound is the docosapotassium salt of a 23-nucleotide (23-mer). In some embodiments, the compound is the tricosapotassium salt of a 24-nucleotide (24-mer). In some embodiments, the compound is the tetracosapotassium salt of a 25-nucleotide (25-mer). In some embodiments, the compound is the pentacosapotassium salt of a 26-nucleotide (26-mer). In some embodiments, the compound is the hexacosapotassium salt of a 27-nucleotide (27-mer). In some embodiments, the compound is the heptacosapotassium salt of a 28-nucleotide (28-mer). In some embodiments, the compound is the octacosapotassium salt of a 29-nucleotide (29-mer). In some embodiments, the compound is the nonacosapotassium salt of a 30-nucleotide (30-mer). In some embodiments, the compound is the triacontapotassium salt of a 31-nucleotide (31-mer). In some embodiments, the compound is the hentriacontapotassium salt of a 32-nucleotide (32-mer). In some embodiments, the compound is the dotriacontapotassium salt of a 33-nucleotide (33-mer). In some embodiments, the compound is the tritriacontapotassium salt of a 34-nucleotide (34-mer). In some embodiments, the compound is the tetratriacontapotassium salt of a 35-nucleotide (35-mer). In some embodiments, the compound is the pentatriacontapotassium salt of a 36-nucleotide (36-mer). In some embodiments, the compound is the hexatriacontapotassium salt of a 37-nucleotide (37-mer). In some embodiments, the compound is the heptatriacontapotassium salt of a 38-nucleotide (38-mer). In some embodiments, the compound is the octatriacontapotassium salt of a 39-nucleotide (39-mer). In some embodiments, the compound is the nonatriacontapotassium salt of a 40-nucleotide (40-mer). In some embodiments, the compound is the tetracontapotassium salt of a 41-nucleotide (41-mer). In some embodiments, the compound is the hentetracontapotassium salt of a 42-nucleotide (42-mer). In some embodiments, the compound is the dotetracontapotassium salt of a 43-nucleotide (43-mer). In some embodiments, the compound is the tritetracontapotassium salt of a 44-nucleotide (44-mer). In some embodiments, the compound is the tetratetracontapotassium salt of a 45-nucleotide (45-mer). In some embodiments, the compound is the pentatetracontapotassium salt of a 46-nucleotide (46-mer). In some embodiments, the compound is the hexatetracontapotassium salt of a 47-nucleotide (47-mer). In some embodiments, the compound is the heptatetracontapotassium salt of a 48-nucleotide (48-mer). In some embodiments, the compound is the octatetracontapotassium salt of a 49-nucleotide (49-mer). In some embodiments, the compound is the nonatetracontapotassium salt of a 50-nucleotide (50-mer). In some embodiments, the compound is the pentacontapotassium salt of a 51-nucleotide (51-mer).

In some embodiments, the compound is the monopotassium salt of a 2-nucleotide (2-mer), fully phosphorothioate-linked oligonucleotide. In some embodiments, the compound is the dipotassium salt of a 3-nucleotide (3-mer), fully phosphorothioate-linked oligonucleotide. In some embodiments, the compound is the tripotassium salt of a 4-nucleotide (4-mer), fully phosphorothioate-linked oligonucleotide. In some embodiments, the compound is the tetrapotassium salt of a 5-nucleotide (5-mer), fully phosphorothioate-linked oligonucleotide. In some embodiments, the compound is the pentapotassium salt of a 6-nucleotide (6-mer), fully phosphorothioate-linked oligonucleotide. In some embodiments, the compound is the hexapotassium salt of a 7-nucleotide (7-mer), fully phosphorothioate-linked oligonucleotide. In some embodiments, the compound is the heptapotassium salt of an 8-nucleotide (8-mer), fully phosphorothioate-linked oligonucleotide. In some embodiments, the compound is the octapotassium salt of a 9-nucleotide (9-mer), fully phosphorothioate-linked oligonucleotide. In some embodiments, the compound is the nonapotassium salt of a 10-nucleotide (10-mer), fully phosphorothioate-linked oligonucleotide. In some embodiments, the compound is the decapotassium salt of a 11-nucleotide (11-mer), fully phosphorothioate-linked oligonucleotide. In some embodiments, the compound is the undecapotassium salt of a 12-nucleotide (12-mer), fully phosphorothioate-linked oligonucleotide. In some embodiments, the compound is the dodecapotassium salt of a 13-nucleotide (13-mer), fully phosphorothioate-linked oligonucleotide. In some embodiments, the compound is the tridecapotassium salt of a 14-nucleotide (14-mer), fully phosphorothioate-linked oligonucleotide. In some embodiments, the compound is the tetradecapotassium salt of a 15-nucleotide (15-mer), fully phosphorothioate-linked oligonucleotide. In some embodiments, the compound is the pentadecapotassium salt of a 16-nucleotide (16-mer), fully phosphorothioate-linked oligonucleotide. In some embodiments, the compound is the hexadecapotassium salt of a 17-nucleotide (17-mer), fully phosphorothioate-linked oligonucleotide. In some embodiments, the compound is the heptadecapotassium salt of an 18-nucleotide (18-mer), fully phosphorothioate-linked oligonucleotide. In some embodiments, the compound is the octadecapotassium salt of a 19-nucleotide (19-mer), fully phosphorothioate-linked oligonucleotide. In some embodiments, the compound is the nonadecapotassium salt of a 20-nucleotide (20-mer), fully phosphorothioate-linked oligonucleotide. In some embodiments, the compound is the icosapotassium salt of a 21-nucleotide (21-mer), fully phosphorothioate-linked oligonucleotide. In some embodiments, the compound is the henicosapotassium salt of a 22-nucleotide (22-mer), fully phosphorothioate-linked oligonucleotide. In some embodiments, the compound is the docosapotassium salt of a 23-nucleotide (23-mer), fully phosphorothioate-linked oligonucleotide. In some embodiments, the compound is the tricosapotassium salt of a 24-nucleotide (24-mer), fully phosphorothioate-linked oligonucleotide. In some embodiments, the compound is the tetracosapotassium salt of a 25-nucleotide (25-mer), fully phosphorothioate-linked oligonucleotide. In some embodiments, the compound is the pentacosapotassium salt of a 26-nucleotide (26-mer), fully phosphorothioate-linked oligonucleotide. In some embodiments, the compound is the hexacosapotassium salt of a 27-nucleotide (27-mer), fully phosphorothioate-linked oligonucleotide. In some embodiments, the compound is the heptacosapotassium salt of a 28-nucleotide (28-mer), fully phosphorothioate-linked oligonucleotide. In some embodiments, the compound is the octacosapotassium salt of a 29-nucleotide (29-mer), fully phosphorothioate-linked oligonucleotide. In some embodiments, the compound is the nonacosapotassium salt of a 30-nucleotide (30-mer), fully phosphorothioate-linked oligonucleotide. In some embodiments, the compound is the triacontapotassium salt of a 31-nucleotide (31-mer), fully phosphorothioate-linked oligonucleotide. In some embodiments, the compound is the hentriacontapotassium salt of a 32-nucleotide (32-mer), fully phosphorothioate-linked oligonucleotide. In some embodiments, the compound is the dotriacontapotassium salt of a 33-nucleotide (33-mer), fully phosphorothioate-linked oligonucleotide. In some embodiments, the compound is the tritriacontapotassium salt of a 34-nucleotide (34-mer), fully phosphorothioate-linked oligonucleotide. In some embodiments, the compound is the tetratriacontapotassium salt of a 35-nucleotide (35-mer), fully phosphorothioate-linked oligonucleotide. In some embodiments, the compound is the pentatriacontapotassium salt of a 36-nucleotide (36-mer), fully phosphorothioate-linked oligonucleotide. In some embodiments, the compound is the hexatriacontapotassium salt of a 37-nucleotide (37-mer), fully phosphorothioate-linked oligonucleotide. In some embodiments, the compound is the heptatriacontapotassium salt of a 38-nucleotide (38-mer), fully phosphorothioate-linked oligonucleotide. In some embodiments, the compound is the octatriacontapotassium salt of a 39-nucleotide (39-mer), fully phosphorothioate-linked oligonucleotide. In some embodiments, the compound is the nonatriacontapotassium salt of a 40-nucleotide (40-mer), fully phosphorothioate-linked oligonucleotide. In some embodiments, the compound is the tetracontapotassium salt of a 41-nucleotide (41-mer), fully phosphorothioate-linked oligonucleotide. In some embodiments, the compound is the hentetracontapotassium salt of a 42-nucleotide (42-mer), fully phosphorothioate-linked oligonucleotide. In some embodiments, the compound is the dotetracontapotassium salt of a 43-nucleotide (43-mer), fully phosphorothioate-linked oligonucleotide. In some embodiments, the compound is the tritetracontapotassium salt of a 44-nucleotide (44-mer), fully phosphorothioate-linked oligonucleotide. In some embodiments, the compound is the tetratetracontapotassium salt of a 45-nucleotide (45-mer), fully phosphorothioate-linked oligonucleotide. In some embodiments, the compound is the pentatetracontapotassium salt of a 46-nucleotide (46-mer), fully phosphorothioate-linked oligonucleotide. In some embodiments, the compound is the hexatetracontapotassium salt of a 47-nucleotide (47-mer), fully phosphorothioate-linked oligonucleotide. In some embodiments, the compound is the heptatetracontapotassium salt of a 48-nucleotide (48-mer), fully phosphorothioate-linked oligonucleotide. In some embodiments, the compound is the octatetracontapotassium salt of a 49-nucleotide (49-mer), fully phosphorothioate-linked oligonucleotide. In some embodiments, the compound is the nonatetracontapotassium salt of a 50-nucleotide (50-mer), fully phosphorothioate-linked oligonucleotide. In some embodiments, the compound is the pentacontapotassium salt of a 51-nucleotide (51-mer), fully phosphorothioate-linked oligonucleotide.

### SCN1A

The SCN1A gene can encode SCN1A (sodium channel, voltage-gated, type I, alpha subunit) protein, which can also be referred to as alpha-subunit of voltage-gated sodium channel Na_{V}1.1. Also described above, SCN1A mutations in DS are spread across the entire protein. More than 100 novel mutations have been identified throughout the gene with the more debilitating arising de novo. These comprise of truncations (47%), missense (43%), deletions (3%), and splice site mutations (7%). The percentage of subjects carrying SCN1A mutations varies between 33 and 100%. The majority of mutations are novel changes (88%).

In some embodiments, the methods described herein are used to modulate, e.g., increase or decrease, the production of a functional Na_{V}1.1 protein. As used herein, the term “functional” refers to the amount of activity or function of a Na_{V}1.1 protein that is necessary to eliminate any one or more symptoms of a treated condition, e.g., Dravet syndrome; Epilepsy, generalized, with febrile seizures plus, type 2; Febrile seizures, familial, 3A; Autism; Epileptic encephalopathy, early infantile, 13; Sick sinus syndrome 1; Alzheimer's disease; or SUDEP. In some embodiments, the methods are used to increase the production of a partially functional Na_{V}1.1 protein. As used herein, the term “partially functional” refers to any amount of activity or function of the Na_{V}1.1 protein that is less than the amount of activity or function that is necessary to eliminate or prevent any one or more symptoms of a disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

In some embodiments, the method is a method of increasing the expression of the Na_{V}1.1 protein by cells of a subject having a NIE containing pre-mRNA encoding the Na_{V}1.1 protein, wherein the human subject has Dravet syndrome characterized by a deficient amount of activity of Na_{V}1.1 protein, and wherein the deficient amount of the Na_{V}1.1 protein is characterized by haploinsufficiency of the Na_{V}1.1 protein. In such an embodiment, the human subject has a first allele encoding a functional Na_{V}1.1 protein, and a second allele from which the Na_{V}1.1 protein is not produced. In another such embodiment, the human subject has a first allele encoding a functional Na_{V}1.1 protein, and a second allele encoding a nonfunctional Na_{V}1.1 protein. In another such embodiment, the human subject has a first allele encoding a functional Na_{V}1.1 protein, and a second allele encoding a partially functional Na_{V}1.1 protein. In some embodiments, the human subject expresses a partially functional Na_{V}1.1 protein from one allele, wherein the partially functional Na_{V}1.1 protein is characterized by a frameshift mutation, a nonsense mutation, a missense mutation, or a partial gene deletion. In some embodiments, the human subject expresses a nonfunctional Na_{V}1.1 protein from one allele, wherein the nonfunctional Na_{V}1.1 protein is characterized by a frameshift mutation, a nonsense mutation, a missense mutation, a partial gene deletion, in one allele. In some embodiments, the human subject has a SCN1A whole gene deletion, in one allele. In any of these embodiments, the antisense oligomer binds to a targeted portion of the NIE containing pre-mRNA transcribed from the second allele, thereby inducing exon skipping of the pseudo-exon from the pre-mRNA, and causing an increase in the level of mature mRNA encoding functional Na_{V}1.1 protein, and an increase in the expression of the Na_{V}1.1 protein in the cells of the human subject.

In embodiments of the present invention, a subject can have a mutation in SCN1A. Mutations in SCN1A can be spread throughout said gene. Na_{V}1.1 protein can consist of four domains. Said SCN1A domains can have transmembrane segments. Mutations in said Na_{V}1.1 protein may arise throughout said protein. Said Na_{V}1.1 protein may consist of at least two isoforms. Mutations in SCN1A may comprise of R931C, R946C, M934I, R1648C, or R1648H. In some cases, mutations may be observed in a C-terminus of a Na_{V}1.1 protein. Mutations in a Na_{V}1.1 protein may also be found in loops between segments 5 and 6 of the first three domains of said Na_{V}1.1 protein. In some cases, mutations may be observed in an N-terminus of a Na_{V}1.1 protein. Exemplary mutations within SCN1A include, but are not limited to, R222X, R712X, I227S, R1892X, W952X, R1245X, R1407X, W1434R, c.4338+1G>A, 51516X, L1670fsX1678, or K1846fsX1856. Mutations that can be targeted with the present invention may also encode a pore of an ion channel.

In some embodiments, the methods and compositions described herein can be used to treat DS. In other embodiments, the methods and compositions described herein can be used to treat severe myclonic epilepsy of infancy (SMEI). In other embodiments, the methods and compositions described herein can be used to treat borderline Dravet syndrome; Epilepsy, generalized, with febrile seizures plus, type 2; Febrile seizures, familial, 3A; Migraine, familial hemiplegic, 3; Autism; Epileptic encephalopathy, early infantile, 13; Sick sinus syndrome 1; Alzheimer's disease or SUDEP.

In related embodiments, the method is a method of using a compound (e.g., compound (I) or a salt thereof, or compound (II)) to increase the expression of a protein or functional RNA. In some embodiments, a compound (e.g., compound (I) or a salt thereof, or compound (II)) is used to increase the expression of Na_{V}1.1 protein in cells of a subject having a NIE containing pre-mRNA encoding Na_{V}1.1 protein, wherein the human subject has a deficiency, e.g., Dravet Syndrome (DS) (also known as SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); or autism, in the amount or function of a Na_{V}1.1 protein. In some embodiments, a compound (e.g., compound (I) or a salt thereof, or compound (II)) is used to increase the expression of Na_{V}1.1 protein in cells of a subject, wherein the human subject has a deficiency, e.g., Epileptic encephalopathy, early infantile, 13; in the amount or function of a SCN8A protein. In some embodiments, a compound (e.g., compound (I) or a salt thereof, or compound (II)) is used to increase the expression of Na_{V}1.1 protein in cells of a subject, wherein the human subject has a deficiency, e.g., Sick sinus syndrome 1; in the amount or function of a SCN5A protein.

In some embodiments, the methods and compositions described herein can also be used to treat borderline SMEI. Additionally, the methods and compositions described herein can be used to treat generalized epilepsy with febrile seizures plus (GEFS+). GEFS+ may be associated with mutations in epilepsy-associated ion channel subunits such as SCN1B or GABRG2. The methods and compositions described herein can also be used to treat sodium channelopathies. Sodium channelopathies may be associated with mutations in SCN1A. Sodium channelopathies may also be associated with subunits of SCN1A, such as the beta subunit, SCN1B. In some cases, additional diseases associated with SCN1A mutations may also be treated with the present disclosure. Related SCN1A diseases associated with SCN1A mutations include, but are not limited to, atypical myotonia congenita, hyperkalemic periodic paralysis, and paramyotonia congenita.

In some embodiments, a subject having any SCN1A mutation known in the art and described in the literature (e.g., by Hamdan, et al., 2009, N. Engl. Med. 360 (6) pp. 599, Mulley, et al., 2005, Hum. Muta. 25, 535-542, of which entire content is incorporated herein by reference) can be treated using the methods and compositions described herein. In some embodiments, the mutation is within any SCN1A intron or exon.

In some embodiments, the NIE containing pre-mRNA transcript that encodes the protein that is causative of the disease or condition is targeted by the ASOs described herein (e.g., compound (I) or a salt thereof, or compound (II)). In some embodiments, a NIE containing pre-mRNA transcript that encodes a protein that is not causative of the disease is targeted by the ASOs. For example, a disease that is the result of a mutation or deficiency of a first protein in a particular pathway may be ameliorated by targeting a NIE containing pre-mRNA that encodes a second protein, thereby increasing production of the second protein. In some embodiments, the function of the second protein is able to compensate for the mutation or deficiency of the first protein (which is causative of the disease or condition).

In some embodiments, the human subject has:
(a) a first mutant allele from which (i) the Na_{V}1.1 protein is produced at a reduced level compared to production from a wild-type allele, (ii) the Na_{V}1.1 protein is produced in a form having reduced function compared to an equivalent wild-type protein, or (iii) the Na_{V}1.1 protein or functional RNA is not produced; and
(b) a second mutant allele from which (i) the Na_{V}1.1 protein is produced at a reduced level compared to production from a wild-type allele, (ii) the Na_{V}1.1 protein is produced in a form having reduced function compared to an equivalent wild-type protein, or (iii) the Na_{V}1.1 protein is not produced, and wherein the NIE containing pre-mRNA is transcribed from the first allele and/or the second allele. In these embodiments, the compound (e.g., compound (I) or a salt thereof, or compound (II)) binds to a targeted portion of the NIE containing pre-mRNA transcribed from the first allele or the second allele, thereby inducing exon skipping of the pseudo-exon from the NIE containing pre-mRNA, and causing an increase in the level of mRNA encoding NaV1.1 protein and an increase in the expression of the target protein or functional RNA in the cells of the human subject. In these embodiments, the target protein or functional RNA having an increase in expression level resulting from the exon skipping of the pseudo-exon from the NIE containing pre-mRNA is either in a form having reduced function compared to the equivalent wild-type protein (partially functional), or having full function compared to the equivalent wild-type protein (fully functional).

In some embodiments, the level of mRNA encoding Na_{V}1.1 protein is increased 1.1 to 10-fold, when compared to the amount of mRNA encoding Na_{V}1.1 protein that is produced in a control cell, e.g., one that is not treated with the antisense oligomer or one that is treated with an antisense oligomer that does not bind to the targeted portion of the SCN1A NIE containing pre-mRNA.

In some embodiments, a subject treated using the methods of the present disclosure expresses a mutant Na_{V}1.1 protein from one allele, wherein the mutant Na_{V}1.1 protein is characterized by a frameshift mutation, a nonsense mutation, a missense mutation, or a partial gene deletion, and wherein the mutant Na_{V}1.1 protein causes an elevated activity level of Na_{V}1.1. In some embodiments, a subject treated using the methods of the present disclosure expresses an elevated amount of Na_{V}1.1 protein from one allele due to a frameshift mutation, a nonsense mutation, a missense mutation, or a partial gene deletion.

In some embodiments, a subject treated using the methods of the present disclosure expresses a partially functional Na_{V}1.1 protein from one allele, wherein the partially functional Na_{V}1.1 protein is characterized by a frameshift mutation, a nonsense mutation, a missense mutation, or a partial gene deletion. In some embodiments, a subject treated using the methods of the disclosure expresses a nonfunctional Na_{V}1.1 protein from one allele, wherein the nonfunctional Na_{V}1.1 protein is characterized by a frameshift mutation, a nonsense mutation, a missense mutation, a partial gene deletion, in one allele. In some embodiments, a subject treated using the methods of the disclosure has a SCN1A whole gene deletion, in one allele.

In some embodiments, the method is a method of decreasing the expression of the Na_{V}1.1 protein by cells of a subject having a NIE containing pre-mRNA encoding the Na_{V}1.1 protein, and wherein the human subject has a gain-of-function mutation in Na_{V}1.1. In such an embodiment, the human subject has an allele from which the Na_{V}1.1 protein is produced in an elevated amount or an allele encoding a mutant SCN1A that induces increased activity of Na_{V}1.1 in the cell. In some embodiments, the increased activity of Na_{V}1.1 is characterized by a prolonged or near persistent sodium current mediated by the mutant Na_{V}1.1 channel, a slowing of fast inactivation, a positive shift in steady-state inactivation, higher channel availability during repetitive stimulation, increased non-inactivated depolarization-induced persistent sodium currents, delayed entry into inactivation, accelerated recovery from fast inactivation, and/or rescue of folding defects by incubation at lower temperature or co-expression of interacting proteins.

### Target Transcripts

Splicing of the identified SCN1A NIE pre-mRNA species to produce functional mature Scn1a mRNA can be induced using a therapeutic agent such as a compound (e.g., an ASO) that stimulates exon skipping of an NIE. Induction of exon skipping can result in inhibition of an NMD pathway. The resulting mature Scn1a mRNA can be translated normally without activating NMD pathway, thereby increasing the amount of Na_{V}1.1 protein in the patient's cells and alleviating symptoms of a condition associated with SCN1A deficiency, such as Dravet Syndrome (DS); Epilepsy, generalized, with febrile seizures plus, type 2; Febrile seizures, familial, 3A; Autism; Epileptic encephalopathy, early infantile, 13; Sick sinus syndrome 1; Alzheimer's disease; or SUDEP.

In various embodiments, the present disclosure provides a therapeutic agent which can target SCN1A pre-mRNA transcripts to modulate, e.g., enhance or inhibit, splicing or protein expression level. The therapeutic agent can be a small molecule, polynucleotide, or polypeptide. In some embodiments, the therapeutic agent is a compound (e.g., compound (I) or a salt thereof, or compound (II)). Various regions or sequences on the SCN1A pre-mRNA can be targeted by a therapeutic agent, such as an ASO. In some embodiments, the compound (e.g., compound (I) or a salt thereof, or compound (II)) targets a SCN1A pre-mRNA transcript containing an NIE. In some embodiments, the compound (e.g., compound (I) or a salt thereof, or compound (II)) targets a sequence within an NIE of a SCN1A pre-mRNA transcript. In some embodiments, the compound targets a sequence upstream (or 5′) from the 5′ end of an NIE (3′ss) of a SCN1A pre-mRNA transcript. In some embodiments, the compound targets a sequence downstream (or 3′) from 3′ end of an NIE (5′ss) of a SCN1A pre-mRNA transcript. In some embodiments, the compound targets a sequence that is within an intron flanking on 5′ end of the NIE of a SCN1A pre-mRNA transcript. In some embodiments, the compound targets a sequence that is within an intron flanking 3′ end of the NIE of a SCN1A pre-mRNA transcript. In some embodiments, the compound targets a sequence comprising an NIE-intron boundary of a SCN1A pre-mRNA transcript. An NIE-intron boundary can refer to the junction of an intron sequence and an NIE region. The intron sequence can flank 5′ end of the NIE, or 3′ end of the NIE. In some embodiments, the compound targets a sequence within an exon of a SCN1A pre-mRNA transcript. In some embodiments, the compound targets a sequence within an intron of a SCN1A pre-mRNA transcript. In some embodiments, the compound targets a sequence comprising both a portion of an intron and a portion of an exon.

In some embodiments, a therapeutic agent described herein modulates binding of a factor involved in splicing of the pre-mRNA containing an NMD exon. In some embodiments, a therapeutic agent described herein interferes with binding of a factor involved in splicing of the pre-mRNA containing an NMD exon. In some embodiments, a therapeutic agent described herein prevents binding of a factor involved in splicing of the pre-mRNA containing an NMD exon. In some embodiments, a therapeutic agent targets a targeted portion located in an intronic region between two canonical exonic regions of the pre-mRNA containing an NMD exon and encoding Na_{V}1.1, and wherein the intronic region contains the NMD exon. In some embodiments, a therapeutic agent targets a targeted portion at least partially overlaps with the NMD exon. In some embodiments, a therapeutic agent targets a targeted portion that is at least partially overlaps with an intron upstream of the NMD exon. In some embodiments, a therapeutic agent targets a targeted portion within the NMD exon.

In some embodiments, a therapeutic agent targets a targeted portion comprising at least about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon. In some embodiments, a therapeutic agent targets a targeted portion comprising at most about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon. In some embodiments, a therapeutic agent targets a targeted portion comprising about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

In some embodiments, a therapeutic agent targets a targeted portion proximal to the NMD exon.

In some embodiments, the compound targets a sequence from about 4 to about 300 nucleotides upstream (or 5′) from the 5′ end of the NIE. In some embodiments, the compound targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, or about 1450 to about 1500 nucleotides upstream (or 5′) from the 5′ end of the NIE region. In some embodiments, the compound may target a sequence more than 300 nucleotides upstream from the 5′ end of the NIE. In some embodiments, the compound targets a sequence from about 4 to about 300 nucleotides downstream (or 3′) from 3′ end of the NIE. In some embodiments, the compound targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, or about 1450 to about 1500 nucleotides downstream from 3′ end of the NIE. In some embodiments, the compound targets a sequence more than 300 nucleotides downstream from 3′ end of the NIE.

In some embodiments, the compound targets a sequence from about 4 to about 300 nucleotides upstream (or 5′) from the 5′ end of the NIE. In some embodiments, the compound targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides upstream (or 5′) from the 5′ end of the NIE region. In some embodiments, the compound targets a sequence about 4 to about 300 nucleotides downstream (or 3′) from 3′ end of the NIE. In some embodiments, the compound targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides downstream from the 3′ end of the NIE. In some embodiments, the compound targets a sequence more than 300 nucleotides downstream from 3′ end of the NIE.

In some embodiments, the compound targets a sequence from about 4 to about 300 nucleotides upstream (or 5′) from the 5′ end of the NIE. In some embodiments, the compound targets a sequence at most about 10 nucleotides, at most about 20 nucleotides, at most about 50 nucleotides, at most about 80 nucleotides, at most about 85 nucleotides, at most about 90 nucleotides, at most about 95 nucleotides, at most about 96 nucleotides, at most about 97 nucleotides, at most about 98 nucleotides, at most about 99 nucleotides, at most about 100 nucleotides, at most about 101 nucleotides, at most about 102 nucleotides, at most about 103 nucleotides, at most about 104 nucleotides, at most about 105 nucleotides, at most about 110 nucleotides, at most about 120 nucleotides, at most about 150 nucleotides, at most about 200 nucleotides, at most about 300 nucleotides, at most about 400 nucleotides, at most about 500 nucleotides, at most about 600 nucleotides, at most about 700 nucleotides, at most about 800 nucleotides, at most about 900 nucleotides, at most about 1000 nucleotides, at most about 1100 nucleotides, at most about 1200 nucleotides, at most about 1300 nucleotides, at most about 1400 nucleotides, or at most about 1500 nucleotides upstream (or 5′) from the 5′ end of the NIE region. In some embodiments, the compound targets a sequence about 4 to about 300 nucleotides downstream (or 3′) from 3′ end of the NIE. In some embodiments, the compound targets a sequence at most about 10 nucleotides, at most about 20 nucleotides, at most about 50 nucleotides, at most about 80 nucleotides, at most about 85 nucleotides, at most about 90 nucleotides, at most about 95 nucleotides, at most about 96 nucleotides, at most about 97 nucleotides, at most about 98 nucleotides, at most about 99 nucleotides, at most about 100 nucleotides, at most about 101 nucleotides, at most about 102 nucleotides, at most about 103 nucleotides, at most about 104 nucleotides, at most about 105 nucleotides, at most about 110 nucleotides, at most about 120 nucleotides, at most about 150 nucleotides, at most about 200 nucleotides, at most about 300 nucleotides, at most about 400 nucleotides, at most about 500 nucleotides, at most about 600 nucleotides, at most about 700 nucleotides, at most about 800 nucleotides, at most about 900 nucleotides, or at most about 1000 nucleotides, at most about 1100 nucleotides, at most about 1200 nucleotides, at most about 1300 nucleotides, at most about 1400 nucleotides, or at most about 1500 nucleotides downstream from 3′ end of the NIE. In some embodiments, the compound targets a sequence more than 300 nucleotides downstream from 3′ end of the NIE.

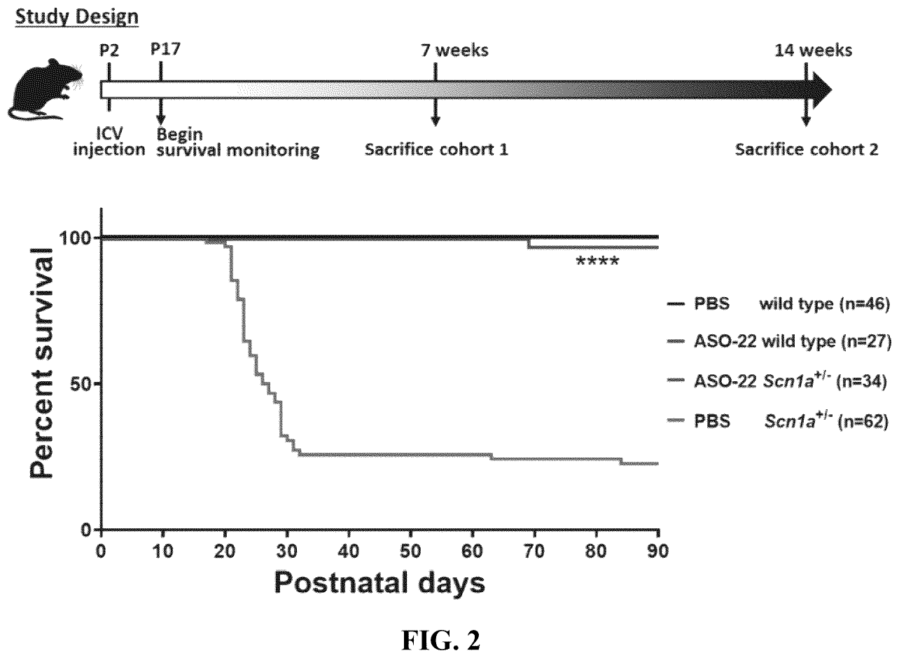

In some embodiments, the NIE as described herein is located between GRCh37/hg19: Chr2:166,863,740 and GRCh37/hg19: Chr2:166,863,803, as depicted in FIG. 2. In some embodiments, 5′ end of the NIE is located at GRCh37/hg19: Chr2:166,863,803. In some embodiments, 3′ end of the NIE is located at GRCh37/hg19: Chr2:166, 863, 740.

In some embodiments, the compound targets a sequence from about 4 to about 300 nucleotides upstream (or 5′) from genomic site GRCh37/hg19: Chr2:166,863,803. In some embodiments, the compound targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, or about 1450 to about 1500 nucleotides upstream (or 5′) from genomic site GRCh37/hg19: Chr2:166,863,803. In some embodiments, the compound may target a sequence more than 300 nucleotides upstream from genomic site GRCh37/hg19: Chr2:166,863,803. In some embodiments, the compound targets a sequence from about 4 to about 300 nucleotides downstream (or 3′) from GRCh37/hg19: Chr2:166,863,740. In some embodiments, the compound targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, or about 1450 to about 1500 nucleotides downstream from GRCh37/hg19: Chr2:166,863,740. In some embodiments, the compound targets a sequence more than 300 nucleotides downstream from GRCh37/hg19: Chr2:166,863,740.

In some embodiments, the compound targets a sequence from about 4 to about 300 nucleotides upstream (or 5′) from genomic site GRCh37/hg19: Chr2:166,863,803. In some embodiments, the compound targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides upstream (or 5′) from genomic site GRCh37/hg19: Chr2:166,863,803. In some embodiments, the compound targets a sequence from about 4 to about 300 nucleotides downstream (or 3′) from GRCh37/hg19: Chr2:166,863,740. In some embodiments, the compound targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides downstream from GRCh37/hg19: Chr2:166,863,740. In some embodiments, the compound targets a sequence more than 300 nucleotides downstream from GRCh37/hg19: Chr2:166,863,740.

In some embodiments, the compound targets a sequence from about 4 to about 300 nucleotides upstream (or 5′) from genomic site GRCh37/hg19: Chr2:166,863,803. In some embodiments, the compound targets a sequence at most about 10 nucleotides, at most about 20 nucleotides, at most about 50 nucleotides, at most about 80 nucleotides, at most about 85 nucleotides, at most about 90 nucleotides, at most about 95 nucleotides, at most about 96 nucleotides, at most about 97 nucleotides, at most about 98 nucleotides, at most about 99 nucleotides, at most about 100 nucleotides, at most about 101 nucleotides, at most about 102 nucleotides, at most about 103 nucleotides, at most about 104 nucleotides, at most about 105 nucleotides, at most about 110 nucleotides, at most about 120 nucleotides, at most about 150 nucleotides, at most about 200 nucleotides, at most about 300 nucleotides, at most about 400 nucleotides, at most about 500 nucleotides, at most about 600 nucleotides, at most about 700 nucleotides, at most about 800 nucleotides, at most about 900 nucleotides, at most about 1000 nucleotides, at most about 1100 nucleotides, at most about 1200 nucleotides, at most about 1300 nucleotides, at most about 1400 nucleotides, or at most about 1500 nucleotides upstream (or 5′) from genomic site GRCh37/hg19: Chr2:166,863,803. In some embodiments, the compound targets a sequence from about 4 to about 300 nucleotides downstream (or 3′) from GRCh37/hg19: Chr2:166,863,740. In some embodiments, the compound targets a sequence at most about 10 nucleotides, at most about 20 nucleotides, at most about 50 nucleotides, at most about 80 nucleotides, at most about 85 nucleotides, at most about 90 nucleotides, at most about 95 nucleotides, at most about 96 nucleotides, at most about 97 nucleotides, at most about 98 nucleotides, at most about 99 nucleotides, at most about 100 nucleotides, at most about 101 nucleotides, at most about 102 nucleotides, at most about 103 nucleotides, at most about 104 nucleotides, at most about 105 nucleotides, at most about 110 nucleotides, at most about 120 nucleotides, at most about 150 nucleotides, at most about 200 nucleotides, at most about 300 nucleotides, at most about 400 nucleotides, at most about 500 nucleotides, at most about 600 nucleotides, at most about 700 nucleotides, at most about 800 nucleotides, at most about 900 nucleotides, or at most about 1000 nucleotides, at most about 1100 nucleotides, at most about 1200 nucleotides, at most about 1300 nucleotides, at most about 1400 nucleotides, or at most about 1500 nucleotides downstream from GRCh37/hg19: Chr2:166,863,740. In some embodiments, the compound targets a sequence more than 300 nucleotides downstream from GRCh37/hg19: Chr2:166,863, 740.

The SCN1A gene (SEQ ID NO: 1) was analyzed for NIE and inclusion of a portion of intron 20 (SEQ ID NO: 4) (this portion is referred as exon 20x throughout the present disclosure) was observed. In some embodiments, the compounds (e.g., ASOs) disclosed herein target a NIE containing pre-mRNA (SEQ ID NO: 2) transcribed from a SCN1A genomic sequence. In some embodiments, the compound targets a NIE containing pre-mRNA transcript from a SCN1A genomic sequence comprising a portion of intron 20. In some embodiments, the compound targets a NIE containing pre-mRNA transcript from a SCN1A genomic sequence comprising exon 20x (SEQ ID NO: 6). In some embodiments, the compound targets a NIE containing pre-mRNA transcript of SEQ ID NO: 2 or 12. In some embodiments, the compound targets a NIE containing pre-mRNA transcript of SEQ ID NO: 2 or 12 comprising an NIE. In some embodiments, the compound targets a NIE containing pre-mRNA transcript of SEQ ID NO: 2 comprising exon 20x (SEQ ID NO: 10). In some embodiments, the compounds disclosed herein target a SCN1A pre-mRNA sequence (SEQ ID NO: 2 or 12). In some embodiments, the compound targets a SCN1A pre-mRNA sequence comprising an NIE (SEQ ID NO: 10 or 20). In some embodiments, the compound targets a SCN1A pre-mRNA sequence according to any one of SEQ ID NOs: 7-10 or 17-20. In some embodiments, the ASO has a sequence according to any one of SEQ ID NOs: 21-67. In some embodiments, the ASO has a sequence according to any one of SEQ ID NOs: 68-114. In some embodiments, the ASO has a sequence according to any one of SEQ ID NOs: 115-209. In some embodiments, the ASO has a sequence according to any one of SEQ ID NOs: 210-256. In some embodiments, the ASO has a sequence according to any one of SEQ ID NOs: 257-303. In some embodiments, the ASO has a sequence according to any one of SEQ ID NOs: 304-341. In some embodiments, the ASO has a sequence according to any one of SEQ ID NOs: 342-379. In some embodiments, the ASO has a sequence according to any one of SEQ ID NOs: 380-1099. In some embodiments, the ASO has a sequence according to any one of SEQ ID NOs: 304-1099. In some embodiments, the ASO has a sequence according to any one of the sequences listed in listed in Tables 4A, 4B, 5A, 5B, 6A, 6B, 7, 8A, and 8B.

In some embodiments, the SCN1A NIE containing pre-mRNA transcript is encoded by a genetic sequence with at least about 80%, 85%, 90%, 95%, 96%, 97%, 98%, 99% or 100% sequence identity to SEQ ID NO: 1 or 11. In some embodiments, the SCN1A NIE pre-mRNA transcript comprises a sequence with at least about 80%, 85%, 90%, 95%, 96%, 97%, 98%, 99% or 100% sequence identity to any one of SEQ ID NOs: 2-10 and 12-20.

In some embodiments, the compound targets exon 20 of a SCN1A NIE containing pre-mRNA comprising NIE exon 20x. In some embodiments, the compound targets an exon 21 sequence downstream (or 3′) of NIE exon 20x. In some embodiments, the compound targets a sequence about 4 to about 300 nucleotides upstream (or 5′) from the 5′ end of exon 20x. In some embodiments, the compound targets a sequence about 4 to about 300 nucleotides downstream (or 3′) from 3′ end of exon 20x. In some embodiments, the ASO has a sequence according to any one of SEQ ID NOs: 21-67. In some embodiments, the ASO has a sequence according to any one of SEQ ID NOs: 210-256. In some embodiments, the ASO has a sequence according to any one of SEQ ID NOs: 380-1099. In some embodiments, the ASO has a sequence according to any one of SEQ ID NOs: 304-1099. In some embodiments, the ASO has a sequence according to any one of the sequences listed in listed in Tables 4A, 4B, 5A, 5B, 6A, 6B, 7, 8A, and 8B.

In some embodiments, the compound targets a sequence upstream from the 5′ end of an NIE. For example, compounds (e.g., ASOs) targeting a sequence upstream from the 5′ end of an NIE (e.g., exon 20x in human SCN1A, or exon 21x in mouse SCN1A) can comprise a sequence with at least 80%, 85%, 90%, 95%, 97%, or 100% sequence identity to any one of SEQ ID NOs: 21-38. For another example, compounds (e.g., ASOs) targeting a sequence upstream from the 5′ end of an NIE (e.g., exon 20x in human SCN1A, or exon 21x in mouse SCN1A) can comprise a sequence with at least 80%, 85%, 90%, 95%, 97%, or 100% sequence identity to any one of SEQ ID NOs: 68-85. In some embodiments, the compounds target a sequence containing an exon-intron boundary (or junction). For example, compounds targeting a sequence containing an exon-intron boundary can comprise a sequence with at least 80%, 85%, 90%, 95%, 97%, or 100% sequence identity to any one of SEQ ID NOs: 39-41, 51, 52, 228-230, 240, or 241. For another example, compounds targeting a sequence containing an exon-intron boundary can comprise a sequence with at least 80%, 85%, 90%, 95%, 97%, or 100% sequence identity to any one of SEQ ID NOs: 86-88 and 98-99. In some embodiments, the compounds target a sequence downstream from 3′ end of an NIE. For example, compounds targeting a sequence down-stream from the 3′ end of an NIE (e.g., exon 20x in human SCN1A, or exon 21x in mouse SCN1A) can comprise a sequence with at least 80%, 85%, 90%, 95%, 97%, or 100% sequence identity to any one of SEQ ID NOs: 53-67. For another example, compounds targeting a sequence downstream from the 3′ end of an NIE (e.g., exon 20x in human SCN1A, or exon 21x in mouse SCN1A) can comprise a sequence with at least 80%, 85%, 90%, 95%, 97%, or 100% sequence identity to any one of SEQ ID NOs: 100-114. In some embodiments, compounds target a sequence within an NIE. For example, compounds targeting a sequence within an NIE (e.g., exon 20x in human SCN1A, or exon 21x in mouse SCN1A) can comprise a sequence with at least 80%, 85%, 90%, 95%, 97%, or 100% sequence identity to any one of SEQ ID NOs: 42-50, or 231-239. For another example, compounds targeting a sequence within an NIE (e.g., exon 20x in human SCN1A, or exon 21x in mouse SCN1A) can comprise a sequence with at least 80%, 85%, 90%, 95%, 97%, or 100% sequence identity to any one of SEQ ID NOs: 89-97.

In some embodiments, the compound (e.g., compound (I) or a salt thereof, or compound (II)) targets exon 20x in a SCN1A NIE containing pre-mRNA comprising exon 20x. In some embodiments, the compound targets an exon 20x sequence downstream (or 3′) from the 5′ end of the exon 20x of a SCN1A pre-mRNA. In some embodiments, the compound targets an exon 20x sequence upstream (or 5′) from 3′ end of the exon 20x of a SCN1A pre-mRNA.

In some embodiments, the SCN1A NIE containing pre-mRNA transcript comprises a sequence with at least about 80%, 85%, 90%, 95%, 97%, or 100% sequence identity to any one of SEQ ID NOs: 2, 7-10, 12, and 17-20. In some embodiments, SCN1A NIE containing pre-mRNA transcript is encoded by a sequence with at least about 80%, 85%, 90%, 95%, 97%, or 100% sequence identity to SEQ ID NOs: 1, 3-6, 11, and 13-16. In some embodiments, the targeted portion of the pre-mRNA containing an NMD exon and encoding Na_{V}1.1 comprises a sequence with at least 80%, 85%, 90%, 95%, 97%, or 100% sequence identity to a region comprising at least 8 contiguous nucleic acids of SEQ ID NOs: 2, 7-10, 12, and 17-20.

In some embodiments, the ASO targets a NIE containing pre-mRNA transcript. In some embodiments, the ASO targets a NIE containing pre-mRNA transcript comprising an NIE. In some embodiments, the ASO targets a NIE containing pre-mRNA transcript of comprising exon 20x. In some embodiments, the ASOs disclosed herein target a SCN1A pre-mRNA sequence. In some embodiments, the ASO targets a SCN1A pre-mRNA sequence comprising an NIE. In some embodiments, the ASO targets a SCN1A pre-mRNA sequence. In some embodiments, the ASO has a sequence according to any one of SEQ ID NOs: 21-67, 210-256 or 304-1099. For another example, the ASOs comprise a sequence with at least 80%, 85%, 90%, 95%, 97%, or 100% sequence identity to any one of SEQ ID NOs: 21-67, 210-256 or 304-1099. For another example, the ASOs comprise a sequence with at least 80%, 85%, 90%, 95%, 97%, or 100% sequence identity to any one of the sequences listed in listed in Tables 4A, 4B, 5A, 5B, 6A, 6B, 7, 8A, and 8B.

In some embodiments, the ASO targets exon 20 of a SCN1A NIE containing pre-mRNA comprising NIE exon 20x. In some embodiments, the ASO targets an exon 21 sequence downstream (or 3′) of NIE exon 20x. In some embodiments, the ASO targets a sequence about 4 to about 300 nucleotides upstream (or 5′) from the 5′ end of exon 20x. In some embodiments, the ASO targets a sequence about 4 to about 300 nucleotides downstream (or 3′) from 3′ end of exon 20x.

In some embodiments, the ASO targets a sequence upstream from the 5′ end of an NIE. In some embodiments, the ASOs target a sequence containing an exon-intron boundary (or junction). In some embodiments, the ASOs target a sequence downstream from 3′ end of an NIE (e.g., exon 20x in human SCN1A, or exon 21x in mouse SCN1A). In some embodiments, ASOs target a sequence within an NIE.

In some embodiments, the ASO targets exon 20x in a SCN1A NIE containing pre-mRNA comprising exon 20x. In some embodiments, the ASO targets an exon 20x sequence downstream (or 3′) from the 5′ end of the exon 20x of a SCN1A pre-mRNA. In some embodiments, the ASO targets an exon 20x sequence upstream (or 5′) from 3′ end of the exon 20x of a SCN1A pre-mRNA.

In some embodiments, the targeted portion of the SCN1A NIE containing pre-mRNA is in intron 1, 2, 3, 4, 5, 6, 7, 8, 9, 10, 11, 12, 13, 14, 15, 16, 17, 18, 19, 20, 21, 22, 23, 24, or 25 (intron numbering corresponding to the mRNA sequence at NM_006920). In some embodiments, hybridization of an ASO to the targeted portion of the NIE pre-mRNA results in exon skipping of at least one of NIE within intron 1, 2, 3, 4, 5, 6, 7, 8, 9, 10, 11, 12, 13, 14, 15, 16, 17, 18, 19, 20, 21, 22, 23, 24, or 25, and subsequently increases Na_{V}1.1 protein production. In some embodiments, hybridization of an ASO to the targeted portion of the NIE pre-mRNA inhibits or blocks exon skipping of at least one of NIE within intron 1, 2, 3, 4, 5, 6, 7, 8, 9, 10, 11, 12, 13, 14, 15, 16, 17, 18, 19, 20, 21, 22, 23, 24, or 25, and subsequently decreases Na_{V}1.1 protein production. In some embodiments, the targeted portion of the SCN1A NIE containing pre-mRNA is in intron 20. One of skill in the art can determine the corresponding intron number in any isoform based on an intron sequence provided herein or using the number provided in reference to the mRNA sequence at NM_006920, NM_001202435, NM_001165964, or NM_001165963. One of skill in the art also can determine the sequences of flanking exons in any SCN1A isoform for targeting using the methods of the invention, based on an intron sequence provided herein or using the intron number provided in reference to the mRNA sequence at NM_006920, NM_001202435, NM_001165964, or NM_001165963.

### Therapeutic Agents

In various embodiments of the present disclosure, compositions and methods comprising a therapeutic agent are provided to modulate protein expression level of SCN1A. In some embodiments, provided herein are compositions and methods to modulate alternative splicing of SCNA1 pre-mRNA. In some embodiments, provided herein are compositions and methods to induce exon skipping in the splicing of SCN1A pre-mRNA, e.g., to induce skipping of a pseudo-exon during splicing of SCN1A pre-mRNA. In other embodiments, therapeutic agents may be used to induce the inclusion of an exon in order to decrease the protein expression level.

In some embodiments, a therapeutic agent disclosed herein is a small molecule, a polypeptide, or a polynucleic acid polymer. In some instances, the therapeutic agent is a small molecule. In some instances, the therapeutic agent is a polypeptide. In some instances, the therapeutic agent is a polynucleic acid polymer. In some cases, the therapeutic agent is a repressor agent. In additional cases, the therapeutic agent is an enhancer agent.

A therapeutic agent disclosed herein can be a NIE repressor agent. A therapeutic agent may comprise a polynucleic acid polymer.

According to one aspect of the present disclosure, provided herein is a method of treatment or prevention of a condition associated with a functional-Na_{V}1.1 protein deficiency, comprising administering a NIE repressor agent to a subject to increase levels of functional Na_{V}1.1 protein, wherein the agent binds to a region of the pre-mRNA transcript to decrease inclusion of the NIE in the mature transcript. For example, provided herein is a method of treatment or prevention of a condition associated with a functional-Na_{V}1.1 protein deficiency, comprising administering a NIE repressor agent to a subject to increase levels of functional Na_{V}1.1 protein, wherein the agent binds to a region of an intron containing an NIE (e.g., intron 20 in human SCN1A gene) of the pre-mRNA transcript or to a NIE-activating regulatory sequence in the same intron.

The sequence of the polynucleic acid polymer may be at least 50%, 55%, 60%, 65%, 70%, 75%, 80%, 85%, 90%, 91%, 92%, 93%, 94%, 95%, 96%, 97%, 98%, 99%, or 99.5% complementary to a target sequence of an mRNA transcript, e.g., a partially processed mRNA transcript. The sequence of the polynucleic acid polymer may be 100% complementary to a target sequence of a pre-mRNA transcript.

The sequence of the polynucleic acid polymer may have 4 or fewer mismatches to a target sequence of the pre-mRNA transcript. The sequence of the polynucleic acid polymer may have 3 or fewer mismatches to a target sequence of the pre-mRNA transcript. The sequence of the polynucleic acid polymer may have 2 or fewer mismatches to a target sequence of the pre-mRNA transcript. The sequence of the polynucleic acid polymer may have 1 or fewer mismatches to a tar-get sequence of the pre-mRNA transcript. The sequence of the polynucleic acid polymer may have no mismatches to a target sequence of the pre-mRNA transcript.

The polynucleic acid polymer may specifically hybridize to a target sequence of the pre-mRNA transcript. For example, the polynucleic acid polymer may have 91%, 92%, 93%, 94%, 95%, 96%, 97%, 98%, 99%, 99.5% or 100% sequence complementarity to a target sequence of the pre-mRNA transcript. The hybridization may be under high stringent hybridization conditions.

The polynucleic acid polymer may have a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of SEQ ID NOs: 21-67. The polynucleic acid polymer may have a sequence with 100% sequence identity to a sequence selected from the group consisting of SEQ ID NOs: 21-67. In some instances, the polynucleic acid polymer may have a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of SEQ ID NOs: 68-114. In some cases, the polynucleic acid polymer may have a sequence with 100% sequence identity to a sequence selected from the group consisting of SEQ ID NOs: 68-114.

In some instances, the polynucleic acid polymer may have a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of SEQ ID NOs: 21-67, 210-256 or 304-1099. In some cases, the polynucleic acid polymer may have a sequence with 100% sequence identity to a sequence selected from the group consisting of SEQ ID NOs: 21-67, 210-256 or 304-1099. In some cases, the polynucleic acid polymer may have a sequence with 100% sequence identity to a sequence selected from the sequences listed in listed in Tables 4A, 4B, 5A, 5B, 6A, 6B, 7, 8A, and 8B.

In embodiments wherein the NIE repressor agent comprises a polynucleic acid polymer, the polynucleic acid polymer may be about 50 nucleotides in length. The polynucleic acid polymer may be about 45 nucleotides in length. The polynucleic acid polymer may be about 40 nucleotides in length. The polynucleic acid polymer may be about 35 nucleotides in length. The polynucleic acid polymer may be about 30 nucleotides in length. The polynucleic acid polymer may be about 24 nucleotides in length. The polynucleic acid polymer may be about 25 nucleotides in length. The polynucleic acid polymer may be about 20 nucleotides in length. The polynucleic acid polymer may be about 19 nucleotides in length. The polynucleic acid polymer may be about 18 nucleotides in length. The polynucleic acid polymer may be about 17 nucleotides in length. The polynucleic acid polymer may be about 16 nucleotides in length. The polynucleic acid polymer may be about 15 nucleotides in length. The polynucleic acid polymer may be about 14 nucleotides in length. The polynucleic acid polymer may be about 13 nucleotides in length. The polynucleic acid polymer may be about 12 nucleotides in length. The polynucleic acid polymer may be about 11 nucleotides in length. The polynucleic acid polymer may be about 10 nucleotides in length. The polynucleic acid polymer may be between about 10 and about 50 nucleotides in length. The polynucleic acid polymer may be between about 10 and about 45 nucleotides in length. The polynucleic acid polymer may be between about 10 and about 40 nucleotides in length. The polynucleic acid polymer may be between about 10 and about 35 nucleotides in length. The polynucleic acid polymer may be between about 10 and about 30 nucleotides in length. The polynucleic acid polymer may be between about 10 and about 25 nucleotides in length. The polynucleic acid polymer may be between about 10 and about 20 nucleotides in length. The polynucleic acid polymer may be between about 15 and about 25 nucleotides in length. The polynucleic acid polymer may be between about 15 and about 30 nucleotides in length. The polynucleic acid polymer may be between about 12 and about 30 nucleotides in length.

The sequence of the polynucleic acid polymer may be at least 50%, 55%, 60%, 65%, 70%, 75%, 80%, 85%, 90%, 91%, 92%, 93%, 94%, 95%, 96%, 97%, 98%, 99%, or 99.5% complementary to a target sequence of an mRNA transcript, e.g., a partially processed mRNA transcript. The sequence of the polynucleic acid polymer may be 100% complementary to a target sequence of a pre-mRNA transcript.

As described herein in various examples, exon 20x in human SCN1A gene is equivalent to exon 21x in mouse SCN1A gene.

Also within the scope of the present disclosure is a method to identify or validate an NMD-inducing exon in the presence of an NMD inhibitor, for example, cycloheximide.

Where reference is made to a polynucleic acid polymer sequence, the skilled person will understand that one or more substitutions may be tolerated, optionally two substitutions may be tolerated in the sequence, such that it maintains the ability to hybridize to the target sequence; or where the substitution is in a target sequence, the ability to be recognized as the target sequence. References to sequence identity may be determined by BLAST sequence alignment using standard/default parameters. For example, the sequence may have 99% identity and still function according to the present disclosure. In other embodiments, the sequence may have 98% identity and still function according to the present disclosure. In another embodiment, the sequence may have 95% identity and still function according to the present disclosure. In another embodiment, the sequence may have 90% identity and still function according to the present disclosure.

### Pharmaceutical Compositions

In some embodiments, the compound as described herein (e.g., compound (I) or a salt thereof, or compound (II)) is solubilized or diluted in an artificial cerebral spinal fluid (aCSF) solution. In some embodiments, the compound as described herein (e.g., compound (I) or a salt thereof, or compound (II)) is solubilized or diluted in an isotonic solution.

The term “artificial cerebrospinal fluid (aCSF),” as used herein, refers to a biological buffer solution that is commonly used as a vehicle solution for administration of agents to the central nervous system (CNS). aCSF, for instance, closely matches the electrolyte concentrations and physiological compatibility of endogenous CSF to enable a vital environment for neuronal tissue by maintaining the homeostasis, osmolarity, and pH at physiological levels.

The term “isotonic solution,” as used herein, refers to a solution that contains an electrolyte balance similar to plasma in the bloodstream. Administration of an isotonic solution to a subject or patient may increase the fluid volume of the human subject or patient without a fluid shift. Exemplary isotonic solutions include, but are not limited to, 0.9% normal saline, lactated Ringer's solution, Ringer's solution, plasmalyte, and 5% Dextrose in water (D₅W).

The term “hypotonic solution,” as used herein, refers to a solution that has a lower concentration of electrolytes than plasma. Administration of a hypotonic solution, for example, via an intravenous route, may lead to shifting fluid out of the bloodstream to the area of higher concentration in the interstitial and intracellular spaces. Exemplary hypotonic solutions include, but are not limited to, 0.45% normal saline (half normal saline), 0.33% NaCl solution, 0.225% NaCl solution, and 2.5% Dextrose in water (D_{2.5}W).

The term “hypertonic solution,” as used herein, refers to a solution that has a higher concentration of electrolytes than plasma. Administration of a hypertonic solution, for example, via an intravenous route, may shift fluid from the interstitial and intracellular spaces into the bloodstream to dilute the electrolytes. Exemplary hypertonic solutions include, but are not limited to, 3% NaCl solution, 5% Dextrose in 0.45% NaCl (D_{5 1/2}NS), 5% Dextrose in 0.9% normal saline (D₅NS), 5% Dextrose in lactated Ringer's solution (D₅LR), 10% Dextrose in water (D₁₀W), 20% Dextrose in water (D₂₀W), and 50% Dextrose in water (D₅₀W).

In some embodiments, the compound as described herein (e.g., compound (I) or a salt thereof, or compound (II)) is solubilized or diluted in a phosphate-buffered (pH 6.6-7.6) solution. In some embodiments, the compound as described herein (e.g., compound (I) or a salt thereof, or compound (II)) is solubilized or diluted in a phosphate-buffered (pH 6.0-8.0) solution. In some embodiments, the compound as described herein (e.g., compound (I) or a salt thereof, or compound (II)) is solubilized or diluted in a phosphate-buffered (pH 5.0-8.0) solution. In some embodiments, the compound as described herein (e.g., compound (I) or a salt thereof, or compound (II)) is solubilized or diluted in a phosphate-buffered solution with pH 4.5-8.5, pH 4.6-8.5, pH 4.7-8.5, pH 4.8-8.5, pH 4.9-8.5, pH5.0-8.5, pH 5.1-8.5, pH5.2-8.5, pH 5.3-8.5, pH5.4-8.5, pH 5.5-8.5, pH5.6-8.5, pH 5.7-8.5, pH 5.8-8.5, H 5.9-8.5, pH 6.0-8.5, pH 6.1-8.5, pH 6.2-8.5, pH 6.3-8.5, pH 6.4-8.5, pH 6.5-8.5, pH 6.6-8.5, pH 6.7-8.5, pH 6.8-8.5, pH 6.9-8.5, pH 7.0-8.5, pH 7.1-8.5, pH 7.2-8.5, pH 7.3-8.5, pH 7.4-8.5, pH 7.5-8.5, pH 7.6-8.5, pH 7.7-8.5, pH 7.8-8.5, pH 7.9-8.5, pH 8.0-8.5, pH 8.1-8.5, pH 8.2-8.5, pH 8.3-8.5, or pH 8.4-8.5. In some embodiments, the compound as described herein (e.g., compound (I) or a salt thereof, or compound (II)) is solubilized or diluted in a phosphate-buffered solution with pH 4.5-8.3, pH 4.5-8.2, pH 4.5-8.1, pH 4.5-8.0, pH 4.5-7.9, pH 4.5-7.8, pH 4.5-7.7, pH 4.5-7.6, pH 4.5-7.5, pH 4.5-7.4, pH 4.5-7.3, pH 4.5-7.2, pH 4.5-7.1, pH 4.5-7.0, pH 4.5-6.9, pH 4.5-6.8, pH 4.5-6.7, pH 4.5-6.6, pH 4.5-6.5, pH 4.5-6.4, pH 4.5-6.3, pH 4.5-6.2, pH 4.5-6.1, pH 4.5-6.0, pH 4.5-5.9, pH 4.5-5.8, pH 4.5-5.7, pH 4.5-5.6, pH 4.5-5.5, pH 4.5-5.4, pH 4.5-5.3, pH 4.5-5.2, pH 4.5-5.1, pH 4.5-5.0, pH 4.5-4.9, pH 4.5-4.8, pH 4.5-4.7, or pH 4.5-4.6. In some embodiments, the compound as described herein (e.g., compound (I) or a salt thereof, or compound (II)) is solubilized or diluted in a phosphate-buffered solution with pH 6.0-7.6, pH 6.1-7.6, pH 6.2-7.6, pH 6.3-7.6, pH 6.4-7.6, pH 6.5-7.6, pH 6.6-7.6, pH 6.7-7.6, pH 6.8-7.6, pH 6.9-7.6, pH 7.0-7.6, pH 7.1-7.6, pH 7.2-7.6, pH 7.3-7.6, pH 7.4-7.6, or pH 7.5-7.6. In some embodiments, the compound as described herein (e.g., compound (I) or a salt thereof, or compound (II)) is solubilized or diluted in a phosphate-buffered solution with pH 6.6-8.0, pH 6.6-7.9, pH 6.6-7.8, pH 6.6-7.7, pH 6.6-7.6, pH 6.6-7.5, pH 6.6-7.4, pH 6.6-7.3, pH 6.6-7.2, pH 6.6-7.1, pH 6.6-7.0, pH 6.6-6.9, pH 6.6-6.8, or pH 6.6-6.7. In some embodiments, the compound as described herein (e.g., compound (I) or a salt thereof, or compound (II)) is solubilized or diluted in a phosphate-buffered solution with pH 6.0-8.0, pH 6.1-8.0, pH 6.2-8.0, pH 6.3-8.0, pH 6.4-8.0, pH 6.5-8.0, pH 6.6-8.0, pH 6.7-8.0, pH 6.8-8.0, pH 6.9-8.0, pH 7.0-8.0, pH 7.1-8.0, pH 7.2-8.0, pH 7.3-8.0, pH 7.4-8.0, pH 7.5-8.0, pH 7.6-8.0, pH 7.7-8.0, pH 7.8-8.0, or pH 7.9-8.0. In some embodiments, the compound as described herein (e.g., compound (I) or a salt thereof, or compound (II)) is solubilized or diluted in a phosphate-buffered solution with pH 6.0-7.9, pH 6.0-7.8, pH 6.0-7.7, pH 6.0-7.6, pH 6.0-7.5, pH 6.0-7.4, pH 6.0-7.3, pH 6.0-7.2, pH 6.0-7.1, pH 6.0-7.0, pH 6.0-6.9, pH 6.0-6.8, pH 6.0-6.7, pH 6.0-6.6, pH 6.0-6.5, pH 6.0-6.4, pH 6.0-6.3, pH 6.0-6.2, or pH 6.0-6.1. In some embodiments, the compound as described herein (e.g., compound (I) or a salt thereof, or compound (II)) is solubilized or diluted in a phosphate-buffered solution with pH 5.7-8.5, 5.8-8.4, 5.9-8.3, 6.0-8.2, 6.1-8.1, 6.2-8.0, 6.3-7.9, 6.4-7.8, 6.5-7.7, or 6.6-7.6. In some embodiments, the compound as described herein (e.g., compound (I) or a salt thereof, or compound (II)) is solubilized or diluted in a phosphate-buffered solution with pH about 5.0, 5.1, 5.2, 5.3, 5.4, 5.5, 5.6, 5.7, 5.8, 5.9, 6.0. 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In some embodiments, the compound as described herein (e.g., compound (I) or a salt thereof, or compound (II)) is solubilized or diluted in a phosphate-buffered solution with pH 5.0, 5.1, 5.2, 5.3, 5.4, 5.5, 5.6, 5.7, 5.8, 5.9, 6.0. 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

In some embodiments, the compound as described herein (e.g., compound (I) or a salt thereof, or compound (II)) is solubilized or diluted in a buffer comprising 25-250 mM NaCl.

In some embodiments, the compound as described herein (e.g., compound (I) or a salt thereof, or compound (II)) is solubilized or diluted in a buffer comprising 25-250, 30-250, 35-250, 40-250, 45-250, 50-250, 55-250, 60-250, 65-250, 70-250, 75-250, 80-250, 85-250, 90-250, 95-250, 100-250, 105-250, 110-250, 115-250, 120-250, 125-250, 130-250, 135-250, 140-250, 145-250, 150-250, 155-250, 160-250, 165-250, 170-250, 175-250, 180-250, 185-250, 190-250, 195-250, 200-250, 205-250, 210-250, 215-250, 220-250, 225-250, 230-250, 235-250, 240-250, or 245-250 mM NaCl. In some embodiments, the compound as described herein (e.g., compound (I) or a salt thereof, or compound (II)) is solubilized or diluted in a buffer comprising 25-245, 25-240, 25-235, 25-230, 25-225, 25-220, 25-215, 25-210, 25-205, 25-200, 25-195, 25-190, 25-185, 25-180, 25-175, 25-170, 25-165, 25-160, 25-155, 25-150, 25-145, 25-140, 25-135, 25-130, 25-125, 25-120, 25-115, 25-110, 25-105, 25-110, 25-105, 25-100, 25-95, 25-90, 25-85, 25-80, 25-75, 25-70, 25-65, 25-60, 25-55, 25-50, 25-45, 25-40, 25-35, or 25-30 mM NaCl. In some embodiments, the compound as described herein (e.g., compound (I) or a salt thereof, or compound (II)) is solubilized or diluted in a buffer comprising 30-245, 35-240, 40-235, 45-230, 50-225, 55-220, 60-215, 65-210, 70-205, 75-200, 80-195, 85-190, 90-185, 95-180, 100-175, 105-170, 110-165, 115-160, 120-155, 125-150, 130-145 or 135-140 mM NaCl. In some embodiments, the compound as described herein (e.g., compound (I) or a salt thereof, or compound (II)) is solubilized or diluted in a buffer comprising 100-140, 101-140, 102-140, 103-140, 104-140, 105-140, 106-140, 107-140, 108-140, 109-140, 110-140, 111-140, 112-140, 113-140, 114-140, 115-140, 116-140, 117-140, 118-140, 119-140, 120-140, 121-140, 122-140, 123-140, 124-140, 125-140, 126-140, 127-140, 128-140, 129-140, 130-140, 131-140, 132-140, 133-140, 134-140, 135-140, 136-140, 137-140, 138-140, or 139-140 mM NaCl. In some embodiments, the compound as described herein (e.g., compound (I) or a salt thereof, or compound (II)) is solubilized or diluted in a buffer comprising 100-139, 100-138, 100-137, 100-136, 100-135, 100-134, 100-133, 100-132, 100-131, 100-130, 100-129, 100-128, 100-127, 100-126, 100-125, 100-124, 100-123, 100-122, 100-121, 100-120, 100-119, 100-118, 100-117, 100-116, 100-115, 100-114, 100-113, 100-112, 100-111, 100-110, 100-109, 100-108, 100-107, 100-106, 100-105, 100-104, 100-103, 100-102, or 100-101 mM NaCl. In some embodiments, the compound as described herein (e.g., compound (I) or a salt thereof, or compound (II)) is solubilized or diluted in a buffer comprising at least 90, 91, 92, 93, 94, 95, 96, 97, 98, 99, 100, 101, 102, 103, 104, 105, 106, 107, 108, 109, 110, 111, 112, 113, 114, 115, 116, 118, 119, 120, 121, 122, 123, 124, 125, 126, 127, 128, 129, 130, 131, 132, 133, 134, 135, 136, 137, 138, 139, or 140 mM NaCl. In some embodiments, the compound as described herein (e.g., compound (I) or a salt thereof, or compound (II)) is solubilized or diluted in a buffer comprising at most 90, 91, 92, 93, 94, 95, 96, 97, 98, 99, 100, 101, 102, 103, 104, 105, 106, 107, 108, 109, 110, 111, 112, 113, 114, 115, 116, 118, 119, 120, 121, 122, 123, 124, 125, 126, 127, 128, 129, 130, 131, 132, 133, 134, 135, 136, 137, 138, 139, or 140 mM NaCl. In some embodiments, the compound as described herein (e.g., compound (I) or a salt thereof, or compound (II)) is solubilized or diluted in a buffer comprising 90, 91, 92, 93, 94, 95, 96, 97, 98, 99, 100, 101, 102, 103, 104, 105, 106, 107, 108, 109, 110, 111, 112, 113, 114, 115, 116, 118, 119, 120, 121, 122, 123, 124, 125, 126, 127, 128, 129, 130, 131, 132, 133, 134, 135, 136, 137, 138, 139, or 140 mM NaCl.

In some embodiments, the compound as described herein (e.g., compound (I) or a salt thereof, or compound (II)) is solubilized or diluted in a buffer comprising 0.1-20 mM KCl.

In some embodiments, the compound as described herein (e.g., compound (I) or a salt thereof, or compound (II)) is solubilized or diluted in a buffer comprising 0.1-40, 0.1-39, 0.1-38, 0.1-37, 0.1-36, 0.1-35, 0.1-34, 0.1-33, 0.1-32, 0.1-31, 0.1-30, 0.1-29, 0.1-28, 0.1-27, 0.1-26, 0.1-25, 0.1-24, 0.1-23, 0.1-22, 0.1-21, 0.1-20, 0.1-19, 0.1-18, 0.1-17, 0.1-16, 0.1-15, 0.1-14, 0.1-13, 0.1-12, 0.1-10, 0.1-9, 0.1-8, 0.1-7, 0.1-6, 0.1-5, 0.1-4, 0.1-3, 0.1-2, or 0.1-1 mM KCl. In some embodiments, the compound as described herein (e.g., compound (I) or a salt thereof, or compound (II)) is solubilized or diluted in a buffer comprising 0.2-40, 0.3-40, 0.4-40, 0.5-40, 0.6-40, 0.7-40, 0.8-40, 0.9-40, 1-40, 2-40, 3-40, 4-40, 5-40, 6-40, 7-40, 8-40, 9-40, 10-40, 11-40, 12-40, 13-40, 14-40, 15-40, 16-40, 17-40, 18-40, 19-40, 20-40, 21-40, 22-40, 23-40, 24-40, 25-40, 26-40, 27-40, 28-40, 29-40, 30-40, 31-40, 32-40, 33-40, 34-40, 35-40, 36-40, 37-40, 38-40, or 39-40 mM KCl. In some embodiments, the compound as described herein (e.g., compound (I) or a salt thereof, or compound (II)) is solubilized or diluted in a buffer comprising 0.1-3.5, 0.2-3.5, 0.3-3.5, 0.4-3.5, 0.5-3.5, 0.6-3.5, 0.7-3.5, 0.8-3.5, 0.9-3.5, 1.0-3.5, 1.1-3.5, 1.2-3.5, 1.3-3.5, 1.4-3.5, 1.5-3.5, 1.6-3.5, 1.7-3.5, 1.8-3.5, 1.9-3.5, 2.0-3.5, 2.1-3.5, 2.2-3.5, 2.3-3.5, 2.4-3.5, 2.5-3.5, 2.6-3.5, 2.7-3.5, 2.8-3.5, 2.9-3.5, 3.0-3.5, 3.1-3.5, 3.2-3.5, 3.3-3.5, or 3.4-3.5 mM KCl. In some embodiments, the compound as described herein (e.g., compound (I) or a salt thereof, or compound (II)) is solubilized or diluted in a buffer comprising 0.1-3.4, 0.1-3.3, 0.1-3.2, 0.1-3.1, 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM KCl. In some embodiments, the compound as described herein (e.g., compound (I) or a salt thereof, or compound (II)) is solubilized or diluted in a buffer comprising at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 mM KCl. In some embodiments, the compound as described herein (e.g., compound (I) or a salt thereof, or compound (II)) is solubilized or diluted in a buffer comprising at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 mM KCl. In some embodiments, the compound as described herein (e.g., compound (I) or a salt thereof, or compound (II)) is solubilized or diluted in a buffer comprising 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 mM KCl.

In some embodiments, the compound as described herein (e.g., compound (I) or a salt thereof, or compound (II)) is solubilized or diluted in a buffer comprising 0.1-50 mM Na₂HPO₄.

In some embodiments, the compound as described herein (e.g., compound (I) or a salt thereof, or compound (II)) is solubilized or diluted in a buffer comprising 0.01-100, 0.02-100, 0.03-100, 0.04-100, 0.05-100, 0.06-100, 0.07-100, 0.08-100, 0.09-100, 0.1-100, 0.2-100, 0.3-100, 0.4-100, 0.5-100, 0.6-100, 0.7-100, 0.8-100, 0.9-100, 1-100, 2-100, 3-100, 4-100, 5-100, 6-100, 7-100, 8-100, 9-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100 mM Na₂HPO₄. In some embodiments, the compound as described herein (e.g., compound (I) or a salt thereof, or compound (II)) is solubilized or diluted in a buffer comprising 0.01-95, 0.01-90, 0.01-85, 0.01-80, 0.01-75, 0.01-70, 0.01-65, 0.01-60, 0.01-55, 0.01-50, 0.01-45, 0.01-40, 0.01-35, 0.01-30, 0.01-25, 0.01-20, 0.01-15, 0.01-10, 0.01-9, 0.01-8, 0.01-7, 0.01-6, 0.01-5, 0.01-4, 0.01-3, 0.01-2, 0.01-1, 0.01-0.9, 0.01-0.8, 0.01-0.7, 0.01-0.6, 0.01-0.5, 0.01-0.4, 0.01-0.3, 0.01-0.2, 0.01-0.1, 0.01-0.09, 0.01-0.08, 0.01-0.07, 0.01-0.06, 0.01-0.05, 0.01-0.04, 0.01-0.03, or 0.01-0.02 mM Na₂HPO₄. In some embodiments, the compound as described herein (e.g., compound (I) or a salt thereof, or compound (II)) is solubilized or diluted in a buffer comprising 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM Na₂HPO₄. In some embodiments, the compound as described herein (e.g., compound (I) or a salt thereof, or compound (II)) is solubilized or diluted in a buffer comprising 0.1-3.0, 0.2-3.0, 0.3-3.0, 0.4-3.0, 0.5-3.0, 0.6-3.0, 0.7-3.0, 0.8-3.0, 0.9-3.0, 1.0-3.0, 1.2-3.0, 1.3-3.0, 1.4-3.0, 1.5-3.0, 1.6-3.0, 1.7-3.0, 1.8-3.0, 1.9-3.0, 2.0-3.0, 2.1-3.0, 2.2-3.0, 2.3-3.0, 2.4-3.0, 2.5-3.0, 2.6-3.0, 2.7-3.0, 2.8-3.0, or 2.9-3.0 mM Na₂HPO₄. In some embodiments, the compound as described herein (e.g., compound (I) or a salt thereof, or compound (II)) is solubilized or diluted in a buffer comprising at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM Na₂HPO₄. In some embodiments, the compound as described herein (e.g., compound (I) or a salt thereof, or compound (II)) is solubilized or diluted in a buffer comprising at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM Na₂HPO₄. In some embodiments, the compound as described herein (e.g., compound (I) or a salt thereof, or compound (II)) is solubilized or diluted in a buffer comprising 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM Na₂HPO₄.

In some embodiments, the compound as described herein (e.g., compound (I) or a salt thereof, or compound (II)) is solubilized or diluted in a buffer comprising 0.1-50 mM NaH₂PO₄.

In some embodiments, the compound as described herein (e.g., compound (I) or a salt thereof, or compound (II)) is solubilized or diluted in a buffer comprising 0.01-100, 0.02-100, 0.03-100, 0.04-100, 0.05-100, 0.06-100, 0.07-100, 0.08-100, 0.09-100, 0.1-100, 0.2-100, 0.3-100, 0.4-100, 0.5-100, 0.6-100, 0.7-100, 0.8-100, 0.9-100, 1-100, 2-100, 3-100, 4-100, 5-100, 6-100, 7-100, 8-100, 9-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100 mM NaH₂PO₄. In some embodiments, the compound as described herein (e.g., compound (I) or a salt thereof, or compound (II)) is solubilized or diluted in a buffer comprising 0.01-95, 0.01-90, 0.01-85, 0.01-80, 0.01-75, 0.01-70, 0.01-65, 0.01-60, 0.01-55, 0.01-50, 0.01-45, 0.01-40, 0.01-35, 0.01-30, 0.01-25, 0.01-20, 0.01-15, 0.01-10, 0.01-9, 0.01-8, 0.01-7, 0.01-6, 0.01-5, 0.01-4, 0.01-3, 0.01-2, 0.01-1, 0.01-0.9, 0.01-0.8, 0.01-0.7, 0.01-0.6, 0.01-0.5, 0.01-0.4, 0.01-0.3, 0.01-0.2, 0.01-0.1, 0.01-0.09, 0.01-0.08, 0.01-0.07, 0.01-0.06, 0.01-0.05, 0.01-0.04, 0.01-0.03, or 0.01-0.02 mM NaH₂PO₄. In some embodiments, the compound as described herein (e.g., compound (I) or a salt thereof, or compound (II)) is solubilized or diluted in a buffer comprising 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM NaH₂PO₄. In some embodiments, the compound as described herein (e.g., compound (I) or a salt thereof, or compound (II)) is solubilized or diluted in a buffer comprising 0.1-3.0, 0.2-3.0, 0.3-3.0, 0.4-3.0, 0.5-3.0, 0.6-3.0, 0.7-3.0, 0.8-3.0, 0.9-3.0, 1.0-3.0, 1.2-3.0, 1.3-3.0, 1.4-3.0, 1.5-3.0, 1.6-3.0, 1.7-3.0, 1.8-3.0, 1.9-3.0, 2.0-3.0, 2.1-3.0, 2.2-3.0, 2.3-3.0, 2.4-3.0, 2.5-3.0, 2.6-3.0, 2.7-3.0, 2.8-3.0, or 2.9-3.0 mM NaH₂PO₄. In some embodiments, the compound as described herein (e.g., compound (I) or a salt thereof, or compound (II)) is solubilized or diluted in a buffer comprising at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM NaH₂PO₄. In some embodiments, the compound as described herein (e.g., compound (I) or a salt thereof, or compound (II)) is solubilized or diluted in a buffer comprising at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM NaH₂PO₄. In some embodiments, the compound as described herein (e.g., compound (I) or a salt thereof, or compound (II)) is solubilized or diluted in a buffer comprising 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM NaH₂PO₄.

In some embodiments, the compound as described herein (e.g., compound (I) or a salt thereof, or compound (II)) is solubilized or diluted in a buffer comprising 0.1-50 mM CaCl₂.

In some embodiments, the compound as described herein (e.g., compound (I) or a salt thereof, or compound (II)) is solubilized or diluted in a buffer comprising 0.1-50, 0.2-50, 0.3-50, 0.4-50, 0.5-50, 0.6-50, 0.7-50, 0.8-50, 0.9-50, 1.0-50, −50, 1.1-50, 1.2-50, 1.3-50, 1.4-50, 1.5-50, 1.6-50, 1.7-50, 1.8-50, 1.9-50, 2.0-50, 2.1-50, 2.2-50, 2.3-50, 2.4-50, 2.5-50, 2.6-50, 2.7-50, 2.8-50, 2.9-50, 3.0-50, 3.1-50, 3.2-50, 3.3-50, 3.4-50, 3.5-50, 3.6-50, 3.7-50, 3.8-50, 3.9-50, 4-50, 5-50, 6-50, 7-50, 8-50, 9-50, 10-50, 15-50, 20-50, 25-50, 1 30-50, 35-50, 40-50, or 45-50 mM CaCl₂. In some embodiments, the compound as described herein (e.g., compound (I) or a salt thereof, or compound (II)) is solubilized or diluted in a buffer comprising 0.1-45, 0.1-40, 0.1-35, 0.1-30, 0.1-25, 0.1-20, 0.1-15, 0.1-10, 0.1-5, 0.1-4, 0.1-4.9, 0.1-4.8, 0.1-4.7, 0.1-4.6, 0.1-4.5, 0.1-4.4, 0.1-4.3, 0.1-4.2, 0.1-4.1, 0.1-4.0, 0.1-3.9, 0.1-3.8, 0.1-3.7, 0.1-3.6, 0.1-3.5, 0.1-3.4, 0.1-3.3, 0.1-3.2, 0.1-3.1, 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM CaCl₂. In some embodiments, the compound as described herein (e.g., compound (I) or a salt thereof, or compound (II)) is solubilized or diluted in a buffer comprising at least 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM CaCl₂. In some embodiments, the compound as described herein (e.g., compound (I) or a salt thereof, or compound (II)) is solubilized or diluted in a buffer comprising at most 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM CaCl₂. In some embodiments, the compound as described herein (e.g., compound (I) or a salt thereof, or compound (II)) is solubilized or diluted in a buffer comprising 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM CaCl₂.

In some embodiments, the compound as described herein (e.g., compound (I) or a salt thereof, or compound (II)) is solubilized or diluted in a buffer comprising 0.1-50 mM MgCl₂.

In some embodiments, the compound as described herein (e.g., compound (I) or a salt thereof, or compound (II)) is solubilized or diluted in a buffer comprising 0.1-50, 0.2-50, 0.3-50, 0.4-50, 0.5-50, 0.6-50, 0.7-50, 0.8-50, 0.9-50, 1.0-50, −50, 1.1-50, 1.2-50, 1.3-50, 1.4-50, 1.5-50, 1.6-50, 1.7-50, 1.8-50, 1.9-50, 2.0-50, 2.1-50, 2.2-50, 2.3-50, 2.4-50, 2.5-50, 2.6-50, 2.7-50, 2.8-50, 2.9-50, 3.0-50, 3.1-50, 3.2-50, 3.3-50, 3.4-50, 3.5-50, 3.6-50, 3.7-50, 3.8-50, 3.9-50, 4-50, 5-50, 6-50, 7-50, 8-50, 9-50, 10-50, 15-50, 20-50, 25-50, 1 30-50, 35-50, 40-50, or 45-50 mM MgCl₂. In some embodiments, the compound as described herein (e.g., compound (I) or a salt thereof, or compound (II)) is solubilized or diluted in a buffer comprising 0.1-45, 0.1-40, 0.1-35, 0.1-30, 0.1-25, 0.1-20, 0.1-15, 0.1-10, 0.1-5, 0.1-4, 0.1-4.9, 0.1-4.8, 0.1-4.7, 0.1-4.6, 0.1-4.5, 0.1-4.4, 0.1-4.3, 0.1-4.2, 0.1-4.1, 0.1-4.0, 0.1-3.9, 0.1-3.8, 0.1-3.7, 0.1-3.6, 0.1-3.5, 0.1-3.4, 0.1-3.3, 0.1-3.2, 0.1-3.1, 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM MgCl₂. In some embodiments, the compound as described herein (e.g., compound (I) or a salt thereof, or compound (II)) is solubilized or diluted in a buffer comprising at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM MgCl₂. In some embodiments, the compound as described herein (e.g., compound (I) or a salt thereof, or compound (II)) is solubilized or diluted in a buffer comprising at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM MgCl₂. In some embodiments, the compound as described herein (e.g., compound (I) or a salt thereof, or compound (II)) is solubilized or diluted in a buffer comprising 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM MgCl₂.

In some embodiments, the compound as described herein (e.g., compound (I) or a salt thereof, or compound (II)) is solubilized or diluted in a buffer further comprising 1-100 mM NaHCO₃, 1-100 mM KHCO₃, or a combination thereof.

In some embodiments, the compound as described herein (e.g., compound (I) or a salt thereof, or compound (II)) is solubilized or diluted in a buffer comprising 1-100 mM NaHCO₃.

In some embodiments, the compound as described herein (e.g., compound (I) or a salt thereof, or compound (II)) is solubilized or diluted in a buffer comprising 1-99, 1-95, 1-90, 1-85, 1-80, 1-75, 1-70, 1-65, 1-60, 1-55, 1-50, 1-45, 1-40, 1-35, 1-30, 1-25, 1-20, 1-15, 1-10, 1-9, 1-8, 1-7, 1-6, 1-5, 1-4, 1-3, or 1-2 mM NaHCO₃. In some embodiments, the compound as described herein (e.g., compound (I) or a salt thereof, or compound (II)) is solubilized or diluted in a buffer comprising 2-100, 3-100, 4-100, 5-100, 6-100, 7-100, 8-100, 9-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100 mM NaHCO₃. In some embodiments, the compound as described herein (e.g., compound (I) or a salt thereof, or compound (II)) is solubilized or diluted in a buffer comprising 24.0-28.0, 24.0-27.9, 24.0-27.8, 24.0-27.7, 24.0-27.6, 24.0-27.5, 24.0-27.4, 24.0-27.3, 24.0-27.2, 24.0-27.1, 24.0-27.0, 24.0-26.9, 24.0-26.8, 24.0-26.7, 24.0-26.6, 24.0-26.5, 24.0-26.4, 24.0-26.3, 24.0-26.2, 24.0-26.1, 24.0-26.0, 24.0-25.9, 4.0-25.8, 24.0-25.7, 24.0-25.6, 24.0-25.5, 24.0-25.4, 24.0-25.3, 24.0-25.2, 24.0-25.1, 24.0-25.0, 24.0-24.9, 24.0-24.8, 24.0-24.7, 24.0-24.6, 24.0-24.5, 24.0-24.4, 24.0-24.3, 24.0-24.2, or 24.0-24.1 mM NaHCO₃. In some embodiments, the compound as described herein (e.g., compound (I) or a salt thereof, or compound (II)) is solubilized or diluted in a buffer comprising 24.1-28.0, 24.2-28.0, 24.3-28.0, 24.4-28.0, 24.5-28.0, 24.6-28.0, 24.7-28.0, 24.8-28.0, 24.9-28.0, 25.0-28.0, 25.1-28.0, 25.2-28.0, 25.3-28.0, 25.4-28.0, 25.5-28.0, 25.6-28.0, 25.7-28.0, 25.8-28.0, 25.9-28.0, 26.0-28.0, 26.1-28.0, 26.2-28.0, 26.3-28.0, 26.4-28.0, 26.5-28.0, 26.6-28.0, 26.7-28.0, 26.8-28.0, 26.9-28.0, 27.0-28.0, 27.1-28.0, 27.2-28.0, 27.3-28.0, 27.4-28.0, 27.5-28.0, 27.6-28.0, 27.7-28.0, 27.8-28.0, or 27.9-28.0 mM NaHCO₃. In some embodiments, the compound as described herein (e.g., compound (I) or a salt thereof, or compound (II)) is solubilized or diluted in a buffer comprising at least 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM NaHCO₃. In some embodiments, the compound as described herein (e.g., compound (I) or a salt thereof, or compound (II)) is solubilized or diluted in a buffer comprising at most 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM NaHCO₃. In some embodiments, the compound as described herein (e.g., compound (I) or a salt thereof, or compound (II)) is solubilized or diluted in a buffer comprising 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM NaHCO₃.

In some embodiments, the compound as described herein (e.g., compound (I) or a salt thereof, or compound (II)) is solubilized or diluted in a buffer comprising 1-100 mM KHCO₃.

In some embodiments, the compound as described herein (e.g., compound (I) or a salt thereof, or compound (II)) is solubilized or diluted in a buffer comprising 1-99, 1-95, 1-90, 1-85, 1-80, 1-75, 1-70, 1-65, 1-60, 1-55, 1-50, 1-45, 1-40, 1-35, 1-30, 1-25, 1-20, 1-15, 1-10, 1-9, 1-8, 1-7, 1-6, 1-5, 1-4, 1-3, or 1-2 mM KHCO₃. In some embodiments, the compound as described herein (e.g., compound (I) or a salt thereof, or compound (II)) is solubilized or diluted in a buffer comprising 2-100, 3-100, 4-100, 5-100, 6-100, 7-100, 8-100, 9-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100 mM KHCO₃. In some embodiments, the compound as described herein (e.g., compound (I) or a salt thereof, or compound (II)) is solubilized or diluted in a buffer comprising 24.0-28.0, 24.0-27.9, 24.0-27.8, 24.0-27.7, 24.0-27.6, 24.0-27.5, 24.0-27.4, 24.0-27.3, 24.0-27.2, 24.0-27.1, 24.0-27.0, 24.0-26.9, 24.0-26.8, 24.0-26.7, 24.0-26.6, 24.0-26.5, 24.0-26.4, 24.0-26.3, 24.0-26.2, 24.0-26.1, 24.0-26.0, 24.0-25.9, 4.0-25.8, 24.0-25.7, 24.0-25.6, 24.0-25.5, 24.0-25.4, 24.0-25.3, 24.0-25.2, 24.0-25.1, 24.0-25.0, 24.0-24.9, 24.0-24.8, 24.0-24.7, 24.0-24.6, 24.0-24.5, 24.0-24.4, 24.0-24.3, 24.0-24.2, or 24.0-24.1 mM KHCO₃. In some embodiments, the compound as described herein (e.g., compound (I) or a salt thereof, or compound (II)) is solubilized or diluted in a buffer comprising 24.1-28.0, 24.2-28.0, 24.3-28.0, 24.4-28.0, 24.5-28.0, 24.6-28.0, 24.7-28.0, 24.8-28.0, 24.9-28.0, 25.0-28.0, 25.1-28.0, 25.2-28.0, 25.3-28.0, 25.4-28.0, 25.5-28.0, 25.6-28.0, 25.7-28.0, 25.8-28.0, 25.9-28.0, 26.0-28.0, 26.1-28.0, 26.2-28.0, 26.3-28.0, 26.4-28.0, 26.5-28.0, 26.6-28.0, 26.7-28.0, 26.8-28.0, 26.9-28.0, 27.0-28.0, 27.1-28.0, 27.2-28.0, 27.3-28.0, 27.4-28.0, 27.5-28.0, 27.6-28.0, 27.7-28.0, 27.8-28.0, or 27.9-28.0 mM KHCO₃. In some embodiments, the compound as described herein (e.g., compound (I) or a salt thereof, or compound (II)) is solubilized or diluted in a buffer comprising at least 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM KHCO₃. In some embodiments, the compound as described herein (e.g., compound (I) or a salt thereof, or compound (II)) is solubilized or diluted in a buffer comprising at most 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM KHCO₃. In some embodiments, the compound as described herein (e.g., compound (I) or a salt thereof, or compound (II)) is solubilized or diluted in a buffer comprising 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM KHCO₃.

In some embodiments, the compound as described herein (e.g., compound (I) or a salt thereof, or compound (II)) is solubilized or diluted in a buffer comprising 0-50 mM KH₂PO₄.

In some embodiments, the compound as described herein (e.g., compound (I) or a salt thereof, or compound (II)) is solubilized or diluted in a buffer comprising 0-100, 0.01-100, 0.02-100, 0.03-100, 0.04-100, 0.05-100, 0.06-100, 0.07-100, 0.08-100, 0.09-100, 0.1-100, 0.2-100, 0.3-100, 0.4-100, 0.5-100, 0.6-100, 0.7-100, 0.8-100, 0.9-100, 1-100, 2-100, 3-100, 4-100, 5-100, 6-100, 7-100, 8-100, 9-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100 mM KH₂PO₄. In some embodiments, the compound as described herein (e.g., compound (I) or a salt thereof, or compound (II)) is solubilized or diluted in a buffer comprising 0-95, 0-90, 0-85, 0-80, 0-75, 0-70, 0-65, 0-60, 0-55, 0-50, 0-45, 0-40, 0-35, 0-30, 0-25, 0-20, 0-15, 0-10, 0-9, 0-8, 0-7, 0-6, 0-5, 0-4, 0-3, 0-2, 0-1, 0-0.9, 0-0.8, 0-0.7, 0-0.6, 0-0.5, 0-0.4, 0-0.3, 0-0.2, 0-0.1, 0-0.09, 0-0.08, 0-0.07, 0-0.06, 0-0.05, 0-0.04, 0-0.03, or 0-0.02 mM KH₂PO₄. In some embodiments, the compound as described herein (e.g., compound (I) or a salt thereof, or compound (II)) is solubilized or diluted in a buffer comprising 0.01-95, 0.01-90, 0.01-85, 0.01-80, 0.01-75, 0.01-70, 0.01-65, 0.01-60, 0.01-55, 0.01-50, 0.01-45, 0.01-40, 0.01-35, 0.01-30, 0.01-25, 0.01-20, 0.01-15, 0.01-10, 0.01-9, 0.01-8, 0.01-7, 0.01-6, 0.01-5, 0.01-4, 0.01-3, 0.01-2, 0.01-1, 0.01-0.9, 0.01-0.8, 0.01-0.7, 0.01-0.6, 0.01-0.5, 0.01-0.4, 0.01-0.3, 0.01-0.2, 0.01-0.1, 0.01-0.09, 0.01-0.08, 0.01-0.07, 0.01-0.06, 0.01-0.05, 0.01-0.04, 0.01-0.03, or 0.01-0.02 mM KH₂PO₄. In some embodiments, the compound as described herein (e.g., compound (I) or a salt thereof, or compound (II)) is solubilized or diluted in a buffer comprising 0-3.0, 0-2.9, 0-2.8, 0-2.7, 0-2.6, 0-2.5, 0-2.4, 0-2.3, 0-2.2, 0-2.1, 0-2.0, 0-1.9, 0-1.8, 0-1.7, 0-1.6, 0-1.5, 0-1.4, 0-1.3, 0-1.2, 0-1.1, 0-1.0, 0-0.9, 0-0.8, 0-0.7, 0-0.6, 0-0.5, 0-0.4, 0-0.3, or 0-0.2 mM KH₂PO₄. In some embodiments, the compound as described herein (e.g., compound (I) or a salt thereof, or compound (II)) is solubilized or diluted in a buffer comprising 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM KH₂PO₄. In some embodiments, the compound as described herein (e.g., compound (I) or a salt thereof, or compound (II)) is solubilized or diluted in a buffer comprising 0-3.0, 0.1-3.0, 0.2-3.0, 0.3-3.0, 0.4-3.0, 0.5-3.0, 0.6-3.0, 0.7-3.0, 0.8-3.0, 0.9-3.0, 1.0-3.0, 1.2-3.0, 1.3-3.0, 1.4-3.0, 1.5-3.0, 1.6-3.0, 1.7-3.0, 1.8-3.0, 1.9-3.0, 2.0-3.0, 2.1-3.0, 2.2-3.0, 2.3-3.0, 2.4-3.0, 2.5-3.0, 2.6-3.0, 2.7-3.0, 2.8-3.0, or 2.9-3.0 mM KH₂PO₄. In some embodiments, the compound as described herein (e.g., compound (I) or a salt thereof, or compound (II)) is solubilized or diluted in a buffer comprising at least 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM KH₂PO₄. In some embodiments, the compound as described herein (e.g., compound (I) or a salt thereof, or compound (II)) is solubilized or diluted in a buffer comprising at most 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM KH₂PO₄. In some embodiments, the compound as described herein (e.g., compound (I) or a salt thereof, or compound (II)) is solubilized or diluted in a buffer comprising 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM KH₂PO₄.

In some embodiments, the compound as described herein (e.g., compound (I) or a salt thereof, or compound (II)) is solubilized or diluted in a buffer comprising 0-50 mM NaH₂PO₄.

In some embodiments, the compound as described herein (e.g., compound (I) or a salt thereof, or compound (II)) is solubilized or diluted in a buffer comprising 0-50, 0-45, 0-40, 0-35, 0-30, 0-25, 0-20, 0-19, 0-18, 0-17, 0-16, 0-15, 0-14, 0-13, 0-12, 0-11, 0-10, 0-9, 0-8, 0-7, 0-6, 0-5, 0-4, 0-3, 0-2, 0-1, 0-0.9, 0-0.8, 0-0.7, 0-0.6, 0-0.5, 0-0.4, 0-0.3, or 0-0.2 mM NaH₂PO₄. In some embodiments, the compound as described herein (e.g., compound (I) or a salt thereof, or compound (II)) is solubilized or diluted in a buffer comprising 0.1-50, 0.1-45, 0.1-40, 0.1-35, 0.1-30, 0.1-25, 0.1-20, 0.1-19, 0.1-18, 0.1-17, 0.1-16, 0.1-15, 0.1-14, 0.1-13, 0.1-12, 0.1-11, 0.1-10, 0.1-9, 0.1-8, 0.1-7, 0.1-6, 0.1-5, 0.1-4, 0.1-3, 0.1-2, 0.1-1, 0.1-0.9, 0.1-0.8, 0.1-0.7, 0.1-0.6, 0.1-0.5, 0.1-0.4, 0.1-0.3, or 0.1-0.2 mM NaH₂PO₄. In some embodiments, the compound as described herein (e.g., compound (I) or a salt thereof, or compound (II)) is solubilized or diluted in a buffer comprising 0-50, 0.1-50, 0.2-50, 0.3-50, 0.4-50, 0.5-50, 0.6-50, 0.7-50, 0.8-50, 0.9-50, 1-50, 2-50, 3-50, 4-50, 5-50, 6-50, 7-50, 8-50, 9-50, 1-50, 11-50, 12-50, 13-50, 14-50, 15-50, 16-50, 17-50, 18-50, 19-50, 20-50, 25-50, 30-50, 35-50, 40-50, or 45-50 mM NaH₂PO₄. In some embodiments, the compound as described herein (e.g., compound (I) or a salt thereof, or compound (II)) is solubilized or diluted in a buffer comprising 0-20, 0.1-20, 0.2-20, 0.3-20, 0.4-20, 0.5-20, 0.6-20, 0.7-20, 0.8-20, 0.9-20, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, or 19-20 mM NaH₂PO₄. In some embodiments, the compound as described herein (e.g., compound (I) or a salt thereof, or compound (II)) is solubilized or diluted in a buffer comprising at least 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mM NaH₂PO₄. In some embodiments, the compound as described herein (e.g., compound (I) or a salt thereof, or compound (II)) is solubilized or diluted in a buffer comprising at most 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mM NaH₂PO₄. In some embodiments, the compound as described herein (e.g., compound (I) or a salt thereof, or compound (II)) is solubilized or diluted in a buffer comprising 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mM NaH₂PO₄.

In some embodiments, the compound as described herein (e.g., compound (I) or a salt thereof, or compound (II)) is solubilized or diluted in a buffer further comprising carbohydrates. In some embodiments, the carbohydrates comprise D-glucose. In some embodiments, the compound as described herein (e.g., compound (I) or a salt thereof, or compound (II)) is solubilized or diluted in a buffer further comprising 1-100 mM D-glucose.

In some embodiments, the compound as described herein (e.g., compound (I) or a salt thereof, or compound (II)) is solubilized or diluted in a buffer comprising 1-100 mM D-glucose.

In some embodiments, the compound as described herein (e.g., compound (I) or a salt thereof, or compound (II)) is solubilized or diluted in a buffer comprising 1-100, 1-95, 1-90, 1-85, 1-80, 1-75, 1-70, 1-65, 1-60, 1-55, 1-50, 1-45, 1-40, 1-35, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 mM D-glucose. In some embodiments, the compound as described herein (e.g., compound (I) or a salt thereof, or compound (II)) is solubilized or diluted in a buffer comprising 2-100, 3-100, 4-100, 5-100, 6-100, 7-100, 8-100, 9-100, 10-100, 11-100, 12-100, 13-100, 14-100, 15-100, 16-100, 17-100, 18-100, 19-100, 20-100, 21-100, 22-100, 23-100, 24-100, 25-100, 26-100, 29-100, 28-100, 29-100, 30-100, 35-100, 40-100, 45-100, 50-100, 55-100, 60-100, 65-100, 70-100, 75-100, 80-100, 85-100, 90-100, or 95-100 mM D-glucose. In some embodiments, the compound as described herein (e.g., compound (I) or a salt thereof, or compound (II)) is solubilized or diluted in a buffer comprising 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 mM D-glucose. In some embodiments, the compound as described herein (e.g., compound (I) or a salt thereof, or compound (II)) is solubilized or diluted in a buffer comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM D-glucose. In some embodiments, the compound as described herein (e.g., compound (I) or a salt thereof, or compound (II)) is solubilized or diluted in a buffer comprising at most 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM D-glucose. In some embodiments, the compound as described herein (e.g., compound (I) or a salt thereof, or compound (II)) is solubilized or diluted in a buffer comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM D-glucose.

In some embodiments, the compound as described herein (e.g., compound (I) or a salt thereof, or compound (II)) is solubilized or diluted in a buffer comprising 1-100, 1-95, 1-90, 1-85, 1-80, 1-75, 1-70, 1-65, 1-60, 1-55, 1-50, 1-45, 1-40, 1-35, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 mM glucose. In some embodiments, the compound as described herein (e.g., compound (I) or a salt thereof, or compound (II)) is solubilized or diluted in a buffer comprising 2-100, 3-100, 4-100, 5-100, 6-100, 7-100, 8-100, 9-100, 10-100, 11-100, 12-100, 13-100, 14-100, 15-100, 16-100, 17-100, 18-100, 19-100, 20-100, 21-100, 22-100, 23-100, 24-100, 25-100, 26-100, 29-100, 28-100, 29-100, 30-100, 35-100, 40-100, 45-100, 50-100, 55-100, 60-100, 65-100, 70-100, 75-100, 80-100, 85-100, 90-100, or 95-100 mM glucose. In some embodiments, the compound as described herein (e.g., compound (I) or a salt thereof, or compound (II)) is solubilized or diluted in a buffer comprising 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 mM glucose. In some embodiments, the compound as described herein (e.g., compound (I) or a salt thereof, or compound (II)) is solubilized or diluted in a buffer comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM glucose. In some embodiments, the compound as described herein (e.g., compound (I) or a salt thereof, or compound (II)) is solubilized or diluted in a buffer comprising at most 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM glucose. In some embodiments, the compound as described herein (e.g., compound (I) or a salt thereof, or compound (II)) is solubilized or diluted in a buffer comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM glucose.

In some embodiments, the compound as described herein (e.g., compound (I) or a salt thereof, or compound (II)) is solubilized or diluted in a buffer comprising 25-250 mM NaCl, 0.1-20 mM KCl, 0.1-50 mM Na₂HPO₄, 0.1-50 mM NaH₂PO₄, 0.1-50 mM CaCl₂, and 0.1-50 mM MgCl₂.

In some embodiments, the compound as described herein (e.g., compound (I) or a salt thereof, or compound (II)) is solubilized or diluted in a buffer comprising 150 mM NaCl, 3.0 mM KCl, 0.7 mM Na₂HPO₄, 0.3 mM NaH₂PO₄, 0.79 mM MgCl₂, and 1.4 mM CaCl₂.

In some embodiments, the compound as described herein (e.g., compound (I) or a salt thereof, or compound (II)) is solubilized or diluted in a buffer further comprising an antioxidant. In some embodiments, the antioxidant is t-butylhydroxyquinoline (TBHQ), butylated hydroxytoluene (BHT), butylated hydroxyanisole (BHA), vitamin E, or any combination thereof. In some embodiments, the compound as described herein (e.g., compound (I) or a salt thereof, or compound (II)) is solubilized or diluted in a buffer further comprising an antioxidant, wherein the antioxidant is ascorbic acid (vitamin C), glutathione, lipoic acid, uric acid, carotenes, α-tocopherol (vitamin E), ubiquinol (coenzyme Q), or any combination thereof.

In some embodiments, the compound as described herein (e.g., compound (I) or a salt thereof, or compound (II)) is solubilized or diluted in a buffer comprising 25-250 mM NaCl, 0.1-20 mM KCl, 0-50 mM KH₂PO₄, 1-100 mM NaHCO₃, 0-50 mM NaH₂PO₄, 1-100 mM D-glucose, 0.1-50 mM CaCl₂, 0.1-50 mM MgCl₂, or any combination thereof.

In some embodiments, the compound as described herein (e.g., compound (I) or a salt thereof, or compound (II)) is solubilized or diluted in a buffer comprising 25-250 mM NaCl, 0.1-20 mM KCl, 0-50 mM KH₂PO₄, 1-100 mM NaHCO₃, 0-50 mM NaH₂PO₄, 1-100 mM D-glucose, 0.1-50 mM CaCl₂, and 0.1-50 mM MgCl₂.

In some embodiments, the compound as described herein (e.g., compound (I) or a salt thereof, or compound (II)) is solubilized or diluted in a buffer comprising 127 mM NaCl, 1.0 mM KCl, 1.2 mM KH₂PO₄, 26 mM NaHCO₃, 10 mM D-glucose, 2.4 mM CaCl₂, and 1.3 mM MgCl₂.

In some embodiments, the compound as described herein (e.g., compound (I) or a salt thereof, or compound (II)) is solubilized or diluted in a buffer comprising 119 mM NaCl, 26.2 mM NaHCO₃, 2.5 mM KCl, 1 mM NaH₂PO₄, 1.3 mM MgCl₂, 10 mM glucose, and 2.5 mM CaCl₂.

In some embodiments, the pharmaceutical composition does not comprise a preservative. In some embodiments, the pharmaceutical composition comprises a preservative.

In some embodiments, the compound as described herein (e.g., compound (I) or a salt thereof, or compound (II)) is present in the pharmaceutical composition at a concentration of from 5-250 mg/mL.

In some embodiments, the compound as described herein (e.g., compound (I) or a salt thereof, or compound (II)) is present in the pharmaceutical composition at a concentration of from 5-250, 5-247.5, 5-245, 5-242.5, 5-240, 5-237.5, 5-235, 5-232.5, 5-230, 5-227.5, 5-225, 5-225.5, 5-220, 5-217.5, 5-215, 5-212.5, 5-210, 5-205.5, 5-205, 5-202.5, 5-200, 5-197.5, 5-195, 5-192.5, 5-190, 5-187.5, 5-185, 5-182.5, 5-180, 5-177.5, 5-175, 5-172.5, 5-170, 5-167.5, 5-165, 5-162.5, 5-160, 5-157.5, 5-155, 5-152.5, 5-150, 5-147.5, 5-145, 5-142.5, 5-140, 5-137.5, 5-135, 5-132.5, 5-130, 5-127.5, 5-125, 5-122.5, 5-120, 5-117.5, 5-115, 5-112.5, 5-110, 5-107.5, 5-105, 5-102.5, 5-100, 5-97.5, 5-95, 5-92.5, 5-90, 5-87.5, 5-85, 5-82.5, 5-80, 5-77.5, 5-75, 5-72.5, 5-70, 5-67.5, 5-65, 5-62.5, 5-60, 5-57.5, 5-55, 5-52.5, 5-50, 5-47.5, 5-45, 5-42.5, 5-40, 5-37.5, 5-35, 5-32.5, 5-30, 5-27.5, 5-25, 5-22.5, 5-20, 5-17.5, 5-15, 5-12.5, or 5-10 mg/mL. In some embodiments, the compound as described herein (e.g., compound (I) or a salt thereof, or compound (II)) is present in the pharmaceutical composition at a concentration of from 10-250, 15-250, 20-250, 25-250, 30-250, 35-250, 40-250, 45-250, 50-250, 55-250, 60-250, 65-250, 70-250, 75-250, 80-250, 85-250, 90-250, 95-250, 100-250, 105-250, 110-250, 115-250, 120-250, 125-250, 130-250, 135-250, 140-250, 145-250, 150-250, 155-250, 160-250, 165-250, 170-250, 175-250, 180-250, 185-250, 190-250, or 195-250, 200-250, 205-250, 210-250, 215-250, 220-250, 225-250, 230-250, 235-250, 240-250, or 245-250 mg/mL. In some embodiments, the compound as described herein (e.g., compound (I) or a salt thereof, or compound (II)) is present in the pharmaceutical composition at a concentration of from at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL. In some embodiments, the compound as described herein (e.g., compound (I) or a salt thereof, or compound (II)) is present in the pharmaceutical composition at a concentration of from at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL. In some embodiments, the compound as described herein (e.g., compound (I) or a salt thereof, or compound (II)) is present in the pharmaceutical composition at a concentration of from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL.

I In some embodiments, the method comprises administering the pharmaceutical composition as a bolus injection. In some embodiments, the method comprises administering the pharmaceutical composition as a bolus injection over 1 to 180 minutes, 175 minutes, 1 to 170 minutes, 1 to 165 minutes, 1 to 160 minutes, 1 to 155 minutes, 1 to 150 minutes, 1 to 145 minutes, 1 to 140 minutes, 1 to 135 minutes, 1 to 130 minutes, 1 to 125 minutes, 1 to 120 minutes, 1 to 115 minutes, 1 to 110 minutes, 1 to 105 minutes, 1 to 100 minutes, 1 to 95 minutes, 1 to 90 minutes, 1 to 85 minutes, 1 to 80 minutes, 1 to 75 minutes, 1 to 70 minutes, 1 to 65 minutes, 1 to 60 minutes, 1 to 55 minutes, 1 to 50 minutes, 1 to 45 minutes, 1 to 40 minutes, 1 to 35 minutes, 1 to 30 minutes, 1 to 25 minutes, 1 to 20 minutes, 1 to 15 minutes, 1 to 10 minutes, 1 to 5 minutes, or 1 to 3 minutes. In some embodiments, the method comprises administering the pharmaceutical composition as a bolus injection using a spinal anesthesia needle.

In some embodiments, the compound as described herein (e.g., compound (I) or a salt thereof, or compound (II)) is present in the pharmaceutical composition at a concentration of from 0.1 mg/mL to 250 mg/mL. In some embodiments, the compound as described herein (e.g., compound (I) or a salt thereof, or compound (II)) is present in the pharmaceutical composition at a concentration of about 0.1 mg/mL, 0.5 mg/mL, 1 mg/mL, 2 mg/mL, 2.5 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, or 20 mg/mL. In some embodiments, the compound as described herein (e.g., compound (I) or a salt thereof, or compound (II)) is present in the pharmaceutical composition at a concentration of about 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL. In some embodiments, the compound as described herein (e.g., compound (I) or a salt thereof, or compound (II)) is present in the pharmaceutical composition at a concentration of about 11 mg/mL, 22 mg/mL, 33 mg/mL, 44 mg/mL, 55 mg/mL, 66 mg/mL, 77 mg/mL, 88 mg/mL, 99 mg/mL, or 100 mg/mL in the diluent.

In some embodiments, the compound as described herein (e.g., compound (I) or a salt thereof, or compound (II)) is present in the pharmaceutical composition at a concentration of from 0.1 mg/mL to 250 mg/mL, from 0.2 mg/mL to 250 mg/mL, from 0.3 mg/mL to 250 mg/mL, from 0.4 mg/mL to 250 mg/mL, from 0.5 mg/mL to 250 mg/mL, from 0.6 mg/mL to 250 mg/mL, from 0.7 mg/mL to 250 mg/mL, from 0.8 mg/mL to 250 mg/mL, from 0.9 mg/ml to 250 mg/mL, from 1.0 mg/mL to 250 mg/mL, from 1.1 mg/mL to 250 mg/mL, from 1.2 mg/mL to 250 mg/mL, from 1.3 mg/mL to 250 mg/mL, from 1.4 mg/mL to 250 mg/mL, from 1.5 mg/mL to 250 mg/mL, from 1.6 mg/mL to 250 mg/mL, from 1.7 mg/mL to 250 mg/mL, from 1.8 mg/mL to 250 mg/mL, from 1.9 mg/mL to 250 mg/mL, from 2.0 mg/mL to 250 mg/mL, from 2.1 mg/mL to 250 mg/mL, from 2.2 mg/mL to 250 mg/mL, from 2.3 mg/mL to 250 mg/mL, from 2.4 mg/mL to 250 mg/mL, from 2.5 mg/mL to 250 mg/mL, from 2.6 mg/mL to 250 mg/mL, from 2.7 mg/mL to 250 mg/mL, from 2.8 mg/mL to 250 mg/mL, from 2.9 mg/mL to 250 mg/mL, from 3.0 mg/mL to 250 mg/mL, from 3.1 mg/mL to 250 mg/mL, from 3.2 mg/mL to 250 mg/mL, from 3.3 mg/mL to 250 mg/mL, from 3.4 mg/mL to 250 mg/mL, from 3.5 mg/mL to 250 mg/mL, from 3.6 mg/mL to 250 mg/mL, from 3.7 mg/mL to 250 mg/mL, from 3.8 mg/mL to 250 mg/mL, from 3.9 mg/mL to 250 mg/mL, from 4.0 mg/mL to 250 mg/mL, from 5.0 mg/mL to 250 mg/mL, from 6.0 mg/mL to 250 mg/mL, from 7.0 mg/mL to 250 mg/mL, from 8.0 mg/mL to 250 mg/mL, from 9.0 mg/mL to 250 mg/mL, from 10 mg/mL to 250 mg/mL, from 15 mg/mL to 250 mg/mL, from 20 mg/mL to 250 mg/mL, from 25 mg/mL to 250 mg/mL, from 30 mg/mL to 250 mg/mL, from 35 mg/mL to 250 mg/mL, from 40 mg/mL to 250 mg/mL, from 45 mg/mL to 250 mg/mL, from 50 mg/mL to 250 mg/mL, from 55 mg/mL to 250 mg/mL, from 60 mg/mL to 250 mg/mL, from 65 mg/mL to 250 mg/mL, from 70 mg/mL to 250 mg/mL, from 75 mg/mL to 250 mg/mL, from 80 mg/mL to 250 mg/mL, from 85 mg/mL to 250 mg/mL, from 90 mg/mL to 250 mg/mL, from 95 mg/mL to 250 mg/mL, from 100 mg/mL to 250 mg/mL, from 105 mg/mL to 250 mg/mL, from 110 mg/mL to 250 mg/mL, from 115 mg/mL to 250 mg/mL, from 120 mg/mL to 250 mg/mL, from 125 mg/mL to 250 mg/mL, from 130 mg/mL to 250 mg/mL, from 135 mg/mL to 250 mg/mL, from 140 mg/mL to 250 mg/mL, from 145 mg/mL to 250 mg/mL, from 150 mg/mL to 250 mg/mL, from 155 mg/mL to 250 mg/mL, from 160 mg/mL to 250 mg/mL, from 165 mg/mL to 250 mg/mL, from 170 mg/mL to 250 mg/mL, from 175 mg/mL to 250 mg/mL, from 180 mg/mL to 250 mg/mL, from 185 mg/mL to 250 mg/mL, from 190 mg/mL to 250 mg/mL, from 195 mg/mL to 250 mg/mL, from 200 mg/mL to 250 mg/mL, from 205 mg/mL to 250 mg/mL, from 210 mg/mL to 250 mg/mL, from 215 mg/mL to 250 mg/mL, from 220 mg/mL to 250 mg/mL, from 225 mg/mL to 250 mg/mL, from 230 mg/mL to 250 mg/mL, from 235 mg/mL to 250 mg/mL, from 240 mg/mL to 250 mg/mL, or from 245 mg/mL to 250 mg/mL.

In some embodiments, the compound as described herein (e.g., compound (I) or a salt thereof, or compound (II)) is present in the pharmaceutical composition at a concentration of from 0.1 mg/mL to 250 mg/mL, from 0.1 mg/mL to 245 mg/mL, from 0.1 mg/mL to 240 mg/mL, from 0.1 mg/mL to 235 mg/mL, from 0.1 mg/mL to 230 mg/mL, from 0.1 mg/mL to 225 mg/mL, from 0.1 mg/mL to 220 mg/mL, from 0.1 mg/mL to 215 mg/mL, from 0.1 mg/mL to 210 mg/mL, from 0.1 mg/mL to 205 mg/mL, from 0.1 mg/mL to 200 mg/mL, from 0.1 mg/mL to 195 mg/mL, from 0.1 mg/mL to 190 mg/mL, from 0.1 mg/mL to 185 mg/mL, from 0.1 mg/mL to 180 mg/mL, from 0.1 mg/mL to 175 mg/mL, from 0.1 mg/mL to 170 mg/mL, from 0.1 mg/mL to 165 mg/mL, from 0.1 mg/mL to 160 mg/mL, from 0.1 mg/mL to 155 mg/mL, from 0.1 mg/mL to 150 mg/mL, from 0.1 mg/mL to 145 mg/mL, from 0.1 mg/mL to 140 mg/mL, from 0.1 mg/mL to 135 mg/mL, from 0.1 mg/mL to 130 mg/mL, from 0.1 mg/mL to 125 mg/mL, from 0.1 mg/mL to 120 mg/mL, from 0.1 mg/mL to 115 mg/mL, from 0.1 mg/mL to 110 mg/mL, from 0.1 mg/mL to 100 mg/mL, from 0.1 mg/mL to 95 mg/mL, from 0.1 mg/mL to 90 mg/mL, from 0.1 mg/mL to 85 mg/mL, from 0.1 mg/mL to 80 mg/mL, from 0.1 mg/mL to 75 mg/mL, from 0.1 mg/mL to 70 mg/mL, from 0.1 mg/mL to 65 mg/mL, from 0.1 mg/mL to 60 mg/mL, from 0.1 mg/mL to 55 mg/mL, from 0.1 mg/mL to 50 mg/mL, from 0.1 mg/mL to 45 mg/mL, from 0.1 mg/mL to 40 mg/mL, from 0.1 mg/mL to 35 mg/mL, from 0.1 mg/mL to 30 mg/mL, from 0.1 mg/mL to 25 mg/mL, from 0.1 mg/mL to 20 mg/mL, from 0.1 mg/mL to 15 mg/mL, from 0.1 mg/mL to 10 mg/mL, from 0.1 mg/mL to 9 mg/mL, from 0.1 mg/mL to 8 mg/mL, from 0.1 mg/mL to 7 mg/mL, from 0.1 mg/mL to 6 mg/mL, from 0.1 mg/mL to 5 mg/mL, from 0.1 mg/mL to 4 mg/mL, from 0.1 mg/mL to 3.9 mg/mL, from 0.1 mg/mL to 3.8 mg/mL, from 0.1 mg/mL to 3.7 mg/mL, from 0.1 mg/mL to 3.6 mg/mL, from 0.1 mg/mL to 3.5 mg/mL, from 0.1 mg/mL to 3.4 mg/mL, from 0.1 mg/mL to 3.3 mg/mL, from 0.1 mg/mL to 3.2 mg/mL, from 0.1 mg/mL to 3.1 mg/mL, from 0.1 mg/mL to 3.0 mg/mL, from 0.1 mg/mL to 2.9 mg/mL, from 0.1 mg/mL to 2.8 mg/mL, from 0.1 mg/mL to 2.7 mg/mL, from 0.1 mg/mL to 2.6 mg/mL, from 0.1 mg/mL to 2.5 mg/mL, from 0.1 mg/mL to 2.4 mg/mL, from 0.1 mg/mL to 2.3 mg/mL, from 0.1 mg/mL to 2.2 mg/mL, from 0.1 mg/mL to 2.1 mg/mL, from 0.1 mg/mL to 2.0 mg/mL, from 0.1 mg/mL to 1.9 mg/mL, from 0.1 mg/mL to 1.8 mg/mL, from 0.1 mg/mL to 1.7 mg/mL, from 0.1 mg/mL to 1.6 mg/mL, from 0.1 mg/mL to 1.5 mg/mL, from 0.1 mg/mL to 1.4 mg/mL, from 0.1 mg/mL to 1.3 mg/mL, from 0.1 mg/mL to 1.2 mg/mL, from 0.1 mg/mL to 1.1 mg/mL, from 0.1 mg/mL to 1.0 mg/mL, from 0.1 mg/mL to 0.9 mg/mL, from 0.1 mg/mL to 0.8 mg/mL, from 0.1 mg/mL to 0.7 mg/mL, from 0.1 mg/mL to 0.6 mg/mL, from 0.1 mg/mL to 0.5 mg/mL, from 0.1 mg/mL to 0.4 mg/mL, from 0.1 mg/mL to 0.3 mg/mL, or from 0.1 mg/mL to 0.2 mg/mL.

In some embodiments, the compound as described herein (e.g., compound (I) or a salt thereof, or compound (II)) is present in the pharmaceutical composition at a concentration of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent. In some embodiments, the compound as described herein (e.g., compound (I) or a salt thereof, or compound (II)) is present in the pharmaceutical composition at a concentration of at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent. In some embodiments, the compound as described herein (e.g., compound (I) or a salt thereof, or compound (II)) is present in the pharmaceutical composition at a concentration of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent.

Pharmaceutical compositions comprising the compound, e.g., antisense oligomer, of the described compositions and for use in any of the described methods can be prepared according to conventional techniques well known in the pharmaceutical industry and described in the published literature. In some embodiments, a pharmaceutical composition for treating a subject comprises an effective amount of any compound as described herein, or a pharmaceutically acceptable salt, solvate, hydrate or ester thereof. In some embodiments, the pharmaceutical composition described herein further comprises a pharmaceutically acceptable excipient, carrier or diluent.

Pharmaceutical compositions can be formulated in a conventional manner using one or more pharmaceutically acceptable inactive ingredients that facilitate processing of the active compounds into preparations that can be used pharmaceutically. A proper formulation is dependent upon the route of administration chosen and a summary of pharmaceutical compositions can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference. In some embodiments, the pharmaceutical composition facilitates administration of the compound to an organism.

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The terms “pharmaceutical composition” and “pharmaceutical formulation” (or “formulation”) are used interchangeably and denote a mixture or solution comprising a therapeutically effective amount of an active pharmaceutical ingredient together with one or more pharmaceutically acceptable excipients to be administered to a subject, e.g., a human in need thereof.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the human subject administered, such as disintegrators, binders, fillers, solvents, buffers, tonicity agents, stabilizers, antioxidants, surfactants, carriers, diluents, excipients, preservatives or lubricants used in formulating pharmaceutical products.

In some embodiments, the compositions are prepared with carriers that will protect the components of the composition against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral anti-gens) can also be used as pharmaceutically acceptable carriers. These can be prepared according to methods known to those skilled in the art, for example, as described in U.S. Pat. No. 4,522,811, of which entire content is incorporated herein by reference.

Pharmaceutical compositions or formulations comprising the compound, e.g., antisense oligomer, of the described compositions and for use in any of the described methods can be prepared according to conventional techniques well known in the pharmaceutical industry and described in the published literature. In some embodiments, a pharmaceutical composition or formulation for treating a subject comprises an effective amount of any compound as described herein, or a pharmaceutically acceptable salt, solvate, hydrate or ester thereof. The pharmaceutical formulation comprising the compound may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66:1-19 (1977), incorporated herein by reference for this purpose. The salts can be prepared in situ during the final isolation and purification of the compounds, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

In some embodiments, provided herein is a method of producing the pharmaceutical composition as described herein.

### Pharmaceutical Formulation

In some aspects, provided herein is a pharmaceutical formulation comprising: a compound (e.g., an ASO) as described herein, wherein the compound as described herein (e.g., compound (I) or a salt thereof, or compound (II)) comprises a sequence with at least 80% sequence identity to any one of SEQ ID NOs: 21-67, 210-256 or 304-1099; and a pharmaceutically acceptable diluent; wherein about 0.1, 0.5, 1, 2.5, 5, 6, 7, 8, 9, 10, 11, 12, 13, 14, 15, 16, 17, 18, 19, 20, 22.5, 25, 27.5, 30, 32.5, 35, 37.5, 40, 42.5, 45, 47.5, 50, 52.5, 55, 57.5, 60, 62.5, 65, 67.5, 70, 72.5, 75, 77.5, 80, 82.5, 85, 87.5, 90, 92.5, 95, 97.5, 100, 102.5, 105, 107.5, 110, 112.5, 115, 117.5, 120, 122.5, 125, 127.5, 130, 132.5, 135, 137.5, 140, 142.5, 145, 147.5, 150, 152.5, 155, 157.5, 160, 162.5, 165, 167.5, 170, 172.5, 175, 177.5, 180, 182.5, 185, 187.5, 190, 192.5, 195, 197.5, 200, 202.5, 205, 207.5, 210, 212.5, 215, 217.5, 220, 222.5, 225, 227.5, 230, 232.5, 235, 237.5, 240, 242.5, 245, 247.5, or 250 mg of the compound as described herein (e.g., compound (I) or a salt thereof, or compound (II)) is dissolved or suspended in a solution at a concentration of from 0.1-250 mg/mL. In some embodiments, the ASO as described herein comprises a sequence with at least 80% sequence identity to any one of the sequences listed in listed in Tables 4A, 4B, 5A, 5B, 6A, 6B, 7, 8A, and 8B.

In some embodiments, the ASO as described herein comprises a sequence with at least 60%, 65%, 66%, 67%, 68%, 69%, 70%, 71%, 72%, 73%, 74%, 75%, 76%, 77%, 78%, 79%, 80%, 81%, 82%, 83%, 884%, 85%, 86%, 87%, 88%, 89%, 90%, 91%, 92%, 93%, 94%, 95%, 96%, 97%, 98%, 99%, 99.5%, 99.8%, 99.9%, or 100% sequence identity to any one of SEQ ID NOs: 21-67, 210-256 or 304-1099. In some embodiments, the ASO as described herein consists of a sequence with at least 60%, 65%, 66%, 67%, 68%, 69%, 70%, 71%, 72%, 73%, 74%, 75%, 76%, 77%, 78%, 79%, 80%, 81%, 82%, 83%, 884%, 85%, 86%, 87%, 88%, 89%, 90%, 91%, 92%, 93%, 94%, 95%, 96%, 97%, 98%, 99%, 99.5%, 99.8%, 99.9%, or 100% sequence identity to any one of SEQ ID NOs: 21-67, 210-256 or 304-1099. In some embodiments, the ASO as described herein consists of a sequence with at least 60%, 65%, 66%, 67%, 68%, 69%, 70%, 71%, 72%, 73%, 74%, 75%, 76%, 77%, 78%, 79%, 80%, 81%, 82%, 83%, 884%, 85%, 86%, 87%, 88%, 89%, 90%, 91%, 92%, 93%, 94%, 95%, 96%, 97%, 98%, 99%, 99.5%, 99.8%, 99.9%, or 100% sequence identity to any one of the sequences listed in listed in Tables 4A, 4B, 5A, 5B, 6A, 6B, 7, 8A, and 8B.

In some embodiments, about 1-500, 2-500, 3-500, 4-500, 5-500, 6-500, 7-500, 8-500, 9-500, 10-500, 15-500, 20-500, 25-500, 30-500, 35-500, 40-500, 45-500, 50-500, 55-500, 60-500, 65-500, 70-500, 75-500, 80-500, 85-500, 90-500, 95-500, 100-500, 105-500, 110-500, 115-500, 120-500, 125-500, 130-500, 135-500, 140-500, 145-500, 150-500, 155-500, 160-500, 165-500, 170-500, 175-500, 180-500, 185-500, 190-500, 195-500, 205-500, 210-500, 215-500, 220-500, 225-500, 230-500, 235-500, 240-500, 245-500, 250-500, 255-500, 260-500, 265-500, 270-500, 275-500, 280-500, 285-500, 290-500, 295-500, 300-500, 305-500, 310-500, 315-500, 320-500, 325-500, 330-500, 335-500, 340-500, 345-500, 350-500, 355-500, 360-500, 365-500, 370-500, 375-500, 380-500, 385-500, 390-500, 395-500, 400-500, 405-500, 410-500, 415-500, 420-500, 425-500, 430-500, 435-500, 440-500, 445-500, 450-500, 455-500, 460-500, 465-500, 470-500, 475-500, 480-500, 485-500, 490-500, or 495-500 mg of the compound as described herein (e.g., compound (I) or a salt thereof, or compound (II)) is dissolved or suspended in a solution at a concentration of from 5-200 mg/mL. In some embodiments, about 1-495, 1-490, 1-485, 1-480, 1-475, 1-470, 1-465, 1-460, 1-455, 1-450, 1-445, 1-440, 1-435, 1-430, 1-425, 1-420, 1-415, 1-410, 1-405, 1-400, 1-395, 1-390, 1-385, 1-380, 1-375, 1-370, 1-365, 1-360, 1-355, 1-350, 1-345, 1-340, 1-335, 1-330, 1-325, 1-320, 1-315, 1-310, 1-305, 1-300, 1-295, 1-290, 1-285, 1-280, 1-275, 1-270, 1-265, 1-260, 1-255, 1-250, 1-245, 1-240, 1-235, 1-230, 1-225, 1-220, 1-215, 1-210, 1-205, 1-200, 1-195, 1-190, 1-185, 1-180, 1-175, 1-170, 1-165, 1-160, 1-155, 1-150, 1-145, 1-140, 1-135, 1-130, 1-125, 1-120, 1-115, 1-110, 1-105, 1-100, 1-95, 1-90, 1-85, 1-80, 1-75, 1-70, 1-65, 1-60, 1-55, 1-0, 1-45, 1-40, 1-35, 1-30, 1-25, 1-20, 1-15, 1-10, 1-9, 1-8, 1-7, 1-6, 1-5, 1-4, 1-3, or 1-2 mg of the compound as described herein (e.g., compound (I) or a salt thereof, or compound (II)) is dissolved or suspended in a solution at a concentration of from 5-200 mg/mL.

In some embodiments, at least about 0.1, 0.5, 1, 2.5, 5, 6, 7, 8, 9, 10, 11, 12, 13, 14, 15, 16, 17, 18, 19, 20, 22.5, 25, 27.5, 30, 32.5, 35, 37.5, 40, 42.5, 45, 47.5, 50, 52.5, 55, 57.5, 60, 62.5, 65, 67.5, 70, 72.5, 75, 77.5, 80, 82.5, 85, 87.5, 90, 92.5, 95, 97.5, 100, 102.5, 105, 107.5, 110, 112.5, 115, 117.5, 120, 122.5, 125, 127.5, 130, 132.5, 135, 137.5, 140, 142.5, 145, 147.5, 150, 152.5, 155, 157.5, 160, 162.5, 165, 167.5, 170, 172.5, 175, 177.5, 180, 182.5, 185, 187.5, 190, 192.5, 195, 197.5, 200, 202.5, 205, 207.5, 210, 212.5, 215, 217.5, 220, 222.5, 225, 227.5, 230, 232.5, 235, 237.5, 240, 242.5, 245, 247.5, or 250 mg of the compound as described herein (e.g., compound (I) or a salt thereof, or compound (II)) is dissolved or suspended in a solution at a concentration of from 0.1-250 mg/mL. In some embodiments, at most about 0.1, 0.5, 1, 2.5, 5, 6, 7, 8, 9, 10, 11, 12, 13, 14, 15, 16, 17, 18, 19, 20, 22.5, 25, 27.5, 30, 32.5, 35, 37.5, 40, 42.5, 45, 47.5, 50, 52.5, 55, 57.5, 60, 62.5, 65, 67.5, 70, 72.5, 75, 77.5, 80, 82.5, 85, 87.5, 90, 92.5, 95, 97.5, 100, 102.5, 105, 107.5, 110, 112.5, 115, 117.5, 120, 122.5, 125, 127.5, 130, 132.5, 135, 137.5, 140, 142.5, 145, 147.5, 150, 152.5, 155, 157.5, 160, 162.5, 165, 167.5, 170, 172.5, 175, 177.5, 180, 182.5, 185, 187.5, 190, 192.5, 195, 197.5, 200, 202.5, 205, 207.5, 210, 212.5, 215, 217.5, 220, 222.5, 225, 227.5, 230, 232.5, 235, 237.5, 240, 242.5, 245, 247.5, or 250 mg of the compound as described herein (e.g., compound (I) or a salt thereof, or compound (II)) is dissolved or suspended in a solution at a concentration of from 0.1-250 mg/mL. In some embodiments, about 0.1, 0.5, 1, 2.5, 5, 6, 7, 8, 9, 10, 11, 12, 13, 14, 15, 16, 17, 18, 19, 20, 22.5, 25, 27.5, 30, 32.5, 35, 37.5, 40, 42.5, 45, 47.5, 50, 52.5, 55, 57.5, 60, 62.5, 65, 67.5, 70, 72.5, 75, 77.5, 80, 82.5, 85, 87.5, 90, 92.5, 95, 97.5, 100, 102.5, 105, 107.5, 110, 112.5, 115, 117.5, 120, 122.5, 125, 127.5, 130, 132.5, 135, 137.5, 140, 142.5, 145, 147.5, 150, 152.5, 155, 157.5, 160, 162.5, 165, 167.5, 170, 172.5, 175, 177.5, 180, 182.5, 185, 187.5, 190, 192.5, 195, 197.5, 200, 202.5, 205, 207.5, 210, 212.5, 215, 217.5, 220, 222.5, 225, 227.5, 230, 232.5, 235, 237.5, 240, 242.5, 245, 247.5, or 250 mg of the compound as described herein (e.g., compound (I) or a salt thereof, or compound (II)) is dissolved or suspended in a solution at a concentration of from 0.1-250 mg/mL.

In some embodiments, the compound as described herein (e.g., compound (I) or a salt thereof, or compound (II)) is dissolved or suspended in a solution at a concentration of from 5-250, 5-247.5, 5-245, 5-242.5, 5-240, 5-237.5, 5-235, 5-232.5, 5-230, 5-227.5, 5-225, 5-225.5, 5-220, 5-217.5, 5-215, 5-212.5, 5-210, 5-205.5, 5-205, 5-202.5, 5-200, 5-197.5, 5-195, 5-192.5, 5-190, 5-187.5, 5-185, 5-182.5, 5-180, 5-177.5, 5-175, 5-172.5, 5-170, 5-167.5, 5-165, 5-162.5, 5-160, 5-157.5, 5-155, 5-152.5, 5-150, 5-147.5, 5-145, 5-142.5, 5-140, 5-137.5, 5-135, 5-132.5, 5-130, 5-127.5, 5-125, 5-122.5, 5-120, 5-117.5, 5-115, 5-112.5, 5-110, 5-107.5, 5-105, 5-102.5, 5-100, 5-97.5, 5-95, 5-92.5, 5-90, 5-87.5, 5-85, 5-82.5, 5-80, 5-77.5, 5-75, 5-72.5, 5-70, 5-67.5, 5-65, 5-62.5, 5-60, 5-57.5, 5-55, 5-52.5, 5-50, 5-47.5, 5-45, 5-42.5, 5-40, 5-37.5, 5-35, 5-32.5, 5-30, 5-27.5, 5-25, 5-22.5, 5-20, 5-17.5, 5-15, 5-12.5, or 5-10 mg/mL. In some embodiments, the compound as described herein (e.g., compound (I) or a salt thereof, or compound (II)) is dissolved or suspended in a solution at a concentration of from 10-250, 15-250, 20-250, 25-250, 30-250, 35-250, 40-250, 45-250, 50-250, 55-250, 60-250, 65-250, 70-250, 75-250, 80-250, 85-250, 90-250, 95-250, 100-250, 105-250, 110-250, 115-250, 120-250, 125-250, 130-250, 135-250, 140-250, 145-250, 150-250, 155-250, 160-250, 165-250, 170-250, 175-250, 180-250, 185-250, 190-250, or 195-250, 200-250, 205-250, 210-250, 215-250, 220-250, 225-250, 230-250, 235-250, 240-250, or 245-250 mg/mL. In some embodiments, the compound as described herein (e.g., compound (I) or a salt thereof, or compound (II)) is dissolved or suspended in a solution at a concentration of from at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL. In some embodiments, the compound as described herein (e.g., compound (I) or a salt thereof, or compound (II)) is dissolved or suspended in a solution at a concentration of from at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL. In some embodiments, the compound as described herein (e.g., compound (I) or a salt thereof, or compound (II)) is dissolved or suspended in a solution at a concentration of from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL.

In some embodiments, the pharmaceutically acceptable diluent comprises an artificial cerebral spinal fluid (aCSF) solution. In some embodiments, the solution comprises a cerebral spinal fluid (CSF) sample from the human subject. In some embodiments, the compound as described herein (e.g., compound (I) or a salt thereof, or compound (II)) is solubilized or diluted in an iso-tonic solution.

In some embodiments, the compound as described herein (e.g., compound (I) or a salt thereof, or compound (II)) is solubilized or diluted in a phosphate-buffered (pH 6.6-7.6) solution. In some embodiments, the compound as described herein (e.g., compound (I) or a salt thereof, or compound (II)) is solubilized or diluted in a phosphate-buffered (pH 6.0-8.0) solution. In some embodiments, the compound as described herein (e.g., compound (I) or a salt thereof, or compound (II)) is solubilized or diluted in a phosphate-buffered (pH 5.0-8.0) solution. In some embodiments, the compound as described herein (e.g., compound (I) or a salt thereof, or compound (II)) is solubilized or diluted in a phosphate-buffered solution with pH 4.5-8.5, pH 4.6-8.5, pH 4.7-8.5, pH 4.8-8.5, pH 4.9-8.5, pH5.0-8.5, pH 5.1-8.5, pH5.2-8.5, pH 5.3-8.5, pH5.4-8.5, pH 5.5-8.5, pH5.6-8.5, pH 5.7-8.5, pH 5.8-8.5, H 5.9-8.5, pH 6.0-8.5, pH 6.1-8.5, pH 6.2-8.5, pH 6.3-8.5, pH 6.4-8.5, pH 6.5-8.5, pH 6.6-8.5, pH 6.7-8.5, pH 6.8-8.5, pH 6.9-8.5, pH 7.0-8.5, pH 7.1-8.5, pH 7.2-8.5, pH 7.3-8.5, pH 7.4-8.5, pH 7.5-8.5, pH 7.6-8.5, pH 7.7-8.5, pH 7.8-8.5, pH 7.9-8.5, pH 8.0-8.5, pH 8.1-8.5, pH 8.2-8.5, pH 8.3-8.5, or pH 8.4-8.5. In some embodiments, the compound as described herein (e.g., compound (I) or a salt thereof, or compound (II)) is solubilized or diluted in a phosphate-buffered solution with pH 4.5-8.3, pH 4.5-8.2, pH 4.5-8.1, pH 4.5-8.0, pH 4.5-7.9, pH 4.5-7.8, pH 4.5-7.7, pH 4.5-7.6, pH 4.5-7.5, pH 4.5-7.4, pH 4.5-7.3, pH 4.5-7.2, pH 4.5-7.1, pH 4.5-7.0, pH 4.5-6.9, pH 4.5-6.8, pH 4.5-6.7, pH 4.5-6.6, pH 4.5-6.5, pH 4.5-6.4, pH 4.5-6.3, pH 4.5-6.2, pH 4.5-6.1, pH 4.5-6.0, pH 4.5-5.9, pH 4.5-5.8, pH 4.5-5.7, pH 4.5-5.6, pH 4.5-5.5, pH 4.5-5.4, pH 4.5-5.3, pH 4.5-5.2, pH 4.5-5.1, pH 4.5-5.0, pH 4.5-4.9, pH 4.5-4.8, pH 4.5-4.7, or pH 4.5-4.6. In some embodiments, the compound as described herein (e.g., compound (I) or a salt thereof, or compound (II)) is solubilized or diluted in a phosphate-buffered solution with pH 6.0-7.6, pH 6.1-7.6, pH 6.2-7.6, pH 6.3-7.6, pH 6.4-7.6, pH 6.5-7.6, pH 6.6-7.6, pH 6.7-7.6, pH 6.8-7.6, pH 6.9-7.6, pH 7.0-7.6, pH 7.1-7.6, pH 7.2-7.6, pH 7.3-7.6, pH 7.4-7.6, or pH 7.5-7.6. In some embodiments, the compound as described herein (e.g., compound (I) or a salt thereof, or compound (II)) is solubilized or diluted in a phosphate-buffered solution with pH 6.6-8.0, pH 6.6-7.9, pH 6.6-7.8, pH 6.6-7.7, pH 6.6-7.6, pH 6.6-7.5, pH 6.6-7.4, pH 6.6-7.3, pH 6.6-7.2, pH 6.6-7.1, pH 6.6-7.0, pH 6.6-6.9, pH 6.6-6.8, or pH 6.6-6.7. In some embodiments, the compound as described herein (e.g., compound (I) or a salt thereof, or compound (II)) is solubilized or diluted in a phosphate-buffered solution with pH 6.0-8.0, pH 6.1-8.0, pH 6.2-8.0, pH 6.3-8.0, pH 6.4-8.0, pH 6.5-8.0, pH 6.6-8.0, pH 6.7-8.0, pH 6.8-8.0, pH 6.9-8.0, pH 7.0-8.0, pH 7.1-8.0, pH 7.2-8.0, pH 7.3-8.0, pH 7.4-8.0, pH 7.5-8.0, pH 7.6-8.0, pH 7.7-8.0, pH 7.8-8.0, or pH 7.9-8.0. In some embodiments, the compound as described herein (e.g., compound (I) or a salt thereof, or compound (II)) is solubilized or diluted in a phosphate-buffered solution with pH 6.0-7.9, pH 6.0-7.8, pH 6.0-7.7, pH 6.0-7.6, pH 6.0-7.5, pH 6.0-7.4, pH 6.0-7.3, pH 6.0-7.2, pH 6.0-7.1, pH 6.0-7.0, pH 6.0-6.9, pH 6.0-6.8, pH 6.0-6.7, pH 6.0-6.6, pH 6.0-6.5, pH 6.0-6.4, pH 6.0-6.3, pH 6.0-6.2, or pH 6.0-6.1. In some embodiments, the compound as described herein (e.g., compound (I) or a salt thereof, or compound (II)) is solubilized or diluted in a phosphate-buffered solution with pH 5.7-8.5, 5.8-8.4, 5.9-8.3, 6.0-8.2, 6.1-8.1, 6.2-8.0, 6.3-7.9, 6.4-7.8, 6.5-7.7, or 6.6-7.6. In some embodiments, the compound as described herein (e.g., compound (I) or a salt thereof, or compound (II)) is solubilized or diluted in a phosphate-buffered solution with pH about 5.0, 5.1, 5.2, 5.3, 5.4, 5.5, 5.6, 5.7, 5.8, 5.9, 6.0. 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In some embodiments, the compound as described herein (e.g., compound (I) or a salt thereof, or compound (II)) is solubilized or diluted in a phosphate-buffered solution with pH 5.0, 5.1, 5.2, 5.3, 5.4, 5.5, 5.6, 5.7, 5.8, 5.9, 6.0. 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

In some embodiments, the compound as described herein (e.g., compound (I) or a salt thereof, or compound (II)) is solubilized or diluted in a buffer comprising 25-250 mM NaCl.

In some embodiments, the compound as described herein (e.g., compound (I) or a salt thereof, or compound (II)) is solubilized or diluted in a buffer comprising 25-250, 30-250, 35-250, 40-250, 45-250, 50-250, 55-250, 60-250, 65-250, 70-250, 75-250, 80-250, 85-250, 90-250, 95-250, 100-250, 105-250, 110-250, 115-250, 120-250, 125-250, 130-250, 135-250, 140-250, 145-250, 150-250, 155-250, 160-250, 165-250, 170-250, 175-250, 180-250, 185-250, 190-250, 195-250, 200-250, 205-250, 210-250, 215-250, 220-250, 225-250, 230-250, 235-250, 240-250, or 245-250 mM NaCl. In some embodiments, the compound as described herein (e.g., compound (I) or a salt thereof, or compound (II)) is solubilized or diluted in a buffer comprising 25-245, 25-240, 25-235, 25-230, 25-225, 25-220, 25-215, 25-210, 25-205, 25-200, 25-195, 25-190, 25-185, 25-180, 25-175, 25-170, 25-165, 25-160, 25-155, 25-150, 25-145, 25-140, 25-135, 25-130, 25-125, 25-120, 25-115, 25-110, 25-105, 25-110, 25-105, 25-100, 25-95, 25-90, 25-85, 25-80, 25-75, 25-70, 25-65, 25-60, 25-55, 25-50, 25-45, 25-40, 25-35, or 25-30 mM NaCl. In some embodiments, the compound as described herein (e.g., compound (I) or a salt thereof, or compound (II)) is solubilized or diluted in a buffer comprising 30-245, 35-240, 40-235, 45-230, 50-225, 55-220, 60-215, 65-210, 70-205, 75-200, 80-195, 85-190, 90-185, 95-180, 100-175, 105-170, 110-165, 115-160, 120-155, 125-150, 130-145 or 135-140 mM NaCl. In some embodiments, the compound as described herein (e.g., compound (I) or a salt thereof, or compound (II)) is solubilized or diluted in a buffer comprising 100-140, 101-140, 102-140, 103-140, 104-140, 105-140, 106-140, 107-140, 108-140, 109-140, 110-140, 111-140, 112-140, 113-140, 114-140, 115-140, 116-140, 117-140, 118-140, 119-140, 120-140, 121-140, 122-140, 123-140, 124-140, 125-140, 126-140, 127-140, 128-140, 129-140, 130-140, 131-140, 132-140, 133-140, 134-140, 135-140, 136-140, 137-140, 138-140, or 139-140 mM NaCl. In some embodiments, the compound as described herein (e.g., compound (I) or a salt thereof, or compound (II)) is solubilized or diluted in a buffer comprising 100-139, 100-138, 100-137, 100-136, 100-135, 100-134, 100-133, 100-132, 100-131, 100-130, 100-129, 100-128, 100-127, 100-126, 100-125, 100-124, 100-123, 100-122, 100-121, 100-120, 100-119, 100-118, 100-117, 100-116, 100-115, 100-114, 100-113, 100-112, 100-111, 100-110, 100-109, 100-108, 100-107, 100-106, 100-105, 100-104, 100-103, 100-102, or 100-101 mM NaCl. In some embodiments, the compound as described herein (e.g., compound (I) or a salt thereof, or compound (II)) is solubilized or diluted in a buffer comprising at least 90, 91, 92, 93, 94, 95, 96, 97, 98, 99, 100, 101, 102, 103, 104, 105, 106, 107, 108, 109, 110, 111, 112, 113, 114, 115, 116, 118, 119, 120, 121, 122, 123, 124, 125, 126, 127, 128, 129, 130, 131, 132, 133, 134, 135, 136, 137, 138, 139, or 140 mM NaCl. In some embodiments, the compound as described herein (e.g., compound (I) or a salt thereof, or compound (II)) is solubilized or diluted in a buffer comprising at most 90, 91, 92, 93, 94, 95, 96, 97, 98, 99, 100, 101, 102, 103, 104, 105, 106, 107, 108, 109, 110, 111, 112, 113, 114, 115, 116, 118, 119, 120, 121, 122, 123, 124, 125, 126, 127, 128, 129, 130, 131, 132, 133, 134, 135, 136, 137, 138, 139, or 140 mM NaCl. In some embodiments, the compound as described herein (e.g., compound (I) or a salt thereof, or compound (II)) is solubilized or diluted in a buffer comprising 90, 91, 92, 93, 94, 95, 96, 97, 98, 99, 100, 101, 102, 103, 104, 105, 106, 107, 108, 109, 110, 111, 112, 113, 114, 115, 116, 118, 119, 120, 121, 122, 123, 124, 125, 126, 127, 128, 129, 130, 131, 132, 133, 134, 135, 136, 137, 138, 139, or 140 mM NaCl.

In some embodiments, the compound as described herein (e.g., compound (I) or a salt thereof, or compound (II)) is solubilized or diluted in a buffer comprising 0.1-20 mM KCl.

In some embodiments, the compound as described herein (e.g., compound (I) or a salt thereof, or compound (II)) is solubilized or diluted in a buffer comprising 0.1-40, 0.1-39, 0.1-38, 0.1-37, 0.1-36, 0.1-35, 0.1-34, 0.1-33, 0.1-32, 0.1-31, 0.1-30, 0.1-29, 0.1-28, 0.1-27, 0.1-26, 0.1-25, 0.1-24, 0.1-23, 0.1-22, 0.1-21, 0.1-20, 0.1-19, 0.1-18, 0.1-17, 0.1-16, 0.1-15, 0.1-14, 0.1-13, 0.1-12, 0.1-10, 0.1-9, 0.1-8, 0.1-7, 0.1-6, 0.1-5, 0.1-4, 0.1-3, 0.1-2, or 0.1-1 mM KCl. In some embodiments, the compound as described herein (e.g., compound (I) or a salt thereof, or compound (II)) is solubilized or diluted in a buffer comprising 0.2-40, 0.3-40, 0.4-40, 0.5-40, 0.6-40, 0.7-40, 0.8-40, 0.9-40, 1-40, 2-40, 3-40, 4-40, 5-40, 6-40, 7-40, 8-40, 9-40, 10-40, 11-40, 12-40, 13-40, 14-40, 15-40, 16-40, 17-40, 18-40, 19-40, 20-40, 21-40, 22-40, 23-40, 24-40, 25-40, 26-40, 27-40, 28-40, 29-40, 30-40, 31-40, 32-40, 33-40, 34-40, 35-40, 36-40, 37-40, 38-40, or 39-40 mM KCl. In some embodiments, the compound as described herein (e.g., compound (I) or a salt thereof, or compound (II)) is solubilized or diluted in a buffer comprising 0.1-3.5, 0.2-3.5, 0.3-3.5, 0.4-3.5, 0.5-3.5, 0.6-3.5, 0.7-3.5, 0.8-3.5, 0.9-3.5, 1.0-3.5, 1.1-3.5, 1.2-3.5, 1.3-3.5, 1.4-3.5, 1.5-3.5, 1.6-3.5, 1.7-3.5, 1.8-3.5, 1.9-3.5, 2.0-3.5, 2.1-3.5, 2.2-3.5, 2.3-3.5, 2.4-3.5, 2.5-3.5, 2.6-3.5, 2.7-3.5, 2.8-3.5, 2.9-3.5, 3.0-3.5, 3.1-3.5, 3.2-3.5, 3.3-3.5, or 3.4-3.5 mM KCl. In some embodiments, the compound as described herein (e.g., compound (I) or a salt thereof, or compound (II)) is solubilized or diluted in a buffer comprising 0.1-3.4, 0.1-3.3, 0.1-3.2, 0.1-3.1, 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM KCl. In some embodiments, the compound as described herein (e.g., compound (I) or a salt thereof, or compound (II)) is solubilized or diluted in a buffer comprising at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 mM KCl. In some embodiments, the compound as described herein (e.g., compound (I) or a salt thereof, or compound (II)) is solubilized or diluted in a buffer comprising at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 mM KCl. In some embodiments, the compound as described herein (e.g., compound (I) or a salt thereof, or compound (II)) is solubilized or diluted in a buffer comprising 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 mM KCl.

In some embodiments, the compound as described herein (e.g., compound (I) or a salt thereof, or compound (II)) is solubilized or diluted in a buffer comprising 0.1-50 mM Na₂HPO₄.

In some embodiments, the compound as described herein (e.g., compound (I) or a salt thereof, or compound (II)) is solubilized or diluted in a buffer comprising 0.01-100, 0.02-100, 0.03-100, 0.04-100, 0.05-100, 0.06-100, 0.07-100, 0.08-100, 0.09-100, 0.1-100, 0.2-100, 0.3-100, 0.4-100, 0.5-100, 0.6-100, 0.7-100, 0.8-100, 0.9-100, 1-100, 2-100, 3-100, 4-100, 5-100, 6-100, 7-100, 8-100, 9-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100 mM Na₂HPO₄. In some embodiments, the compound as described herein (e.g., compound (I) or a salt thereof, or compound (II)) is solubilized or diluted in a buffer comprising 0.01-95, 0.01-90, 0.01-85, 0.01-80, 0.01-75, 0.01-70, 0.01-65, 0.01-60, 0.01-55, 0.01-50, 0.01-45, 0.01-40, 0.01-35, 0.01-30, 0.01-25, 0.01-20, 0.01-15, 0.01-10, 0.01-9, 0.01-8, 0.01-7, 0.01-6, 0.01-5, 0.01-4, 0.01-3, 0.01-2, 0.01-1, 0.01-0.9, 0.01-0.8, 0.01-0.7, 0.01-0.6, 0.01-0.5, 0.01-0.4, 0.01-0.3, 0.01-0.2, 0.01-0.1, 0.01-0.09, 0.01-0.08, 0.01-0.07, 0.01-0.06, 0.01-0.05, 0.01-0.04, 0.01-0.03, or 0.01-0.02 mM Na₂HPO₄. In some embodiments, the compound as described herein (e.g., compound (I) or a salt thereof, or compound (II)) is solubilized or diluted in a buffer comprising 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM Na₂HPO₄. In some embodiments, the compound as described herein (e.g., compound (I) or a salt thereof, or compound (II)) is solubilized or diluted in a buffer comprising 0.1-3.0, 0.2-3.0, 0.3-3.0, 0.4-3.0, 0.5-3.0, 0.6-3.0, 0.7-3.0, 0.8-3.0, 0.9-3.0, 1.0-3.0, 1.2-3.0, 1.3-3.0, 1.4-3.0, 1.5-3.0, 1.6-3.0, 1.7-3.0, 1.8-3.0, 1.9-3.0, 2.0-3.0, 2.1-3.0, 2.2-3.0, 2.3-3.0, 2.4-3.0, 2.5-3.0, 2.6-3.0, 2.7-3.0, 2.8-3.0, or 2.9-3.0 mM Na₂HPO₄. In some embodiments, the compound as described herein (e.g., compound (I) or a salt thereof, or compound (II)) is solubilized or diluted in a buffer comprising at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM Na₂HPO₄. In some embodiments, the compound as described herein (e.g., compound (I) or a salt thereof, or compound (II)) is solubilized or diluted in a buffer comprising at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM Na₂HPO₄. In some embodiments, the compound as described herein (e.g., compound (I) or a salt thereof, or compound (II)) is solubilized or diluted in a buffer comprising 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM Na₂HPO₄.

In some embodiments, the compound as described herein (e.g., compound (I) or a salt thereof, or compound (II)) is solubilized or diluted in a buffer comprising 0.1-50 mM NaH₂PO₄.

In some embodiments, the compound as described herein (e.g., compound (I) or a salt thereof, or compound (II)) is solubilized or diluted in a buffer comprising 0.01-100, 0.02-100, 0.03-100, 0.04-100, 0.05-100, 0.06-100, 0.07-100, 0.08-100, 0.09-100, 0.1-100, 0.2-100, 0.3-100, 0.4-100, 0.5-100, 0.6-100, 0.7-100, 0.8-100, 0.9-100, 1-100, 2-100, 3-100, 4-100, 5-100, 6-100, 7-100, 8-100, 9-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100 mM NaH₂PO₄. In some embodiments, the compound as described herein (e.g., compound (I) or a salt thereof, or compound (II)) is solubilized or diluted in a buffer comprising 0.01-95, 0.01-90, 0.01-85, 0.01-80, 0.01-75, 0.01-70, 0.01-65, 0.01-60, 0.01-55, 0.01-50, 0.01-45, 0.01-40, 0.01-35, 0.01-30, 0.01-25, 0.01-20, 0.01-15, 0.01-10, 0.01-9, 0.01-8, 0.01-7, 0.01-6, 0.01-5, 0.01-4, 0.01-3, 0.01-2, 0.01-1, 0.01-0.9, 0.01-0.8, 0.01-0.7, 0.01-0.6, 0.01-0.5, 0.01-0.4, 0.01-0.3, 0.01-0.2, 0.01-0.1, 0.01-0.09, 0.01-0.08, 0.01-0.07, 0.01-0.06, 0.01-0.05, 0.01-0.04, 0.01-0.03, or 0.01-0.02 mM NaH₂PO₄. In some embodiments, the compound as described herein (e.g., compound (I) or a salt thereof, or compound (II)) is solubilized or diluted in a buffer comprising 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM NaH₂PO₄. In some embodiments, the compound as described herein (e.g., compound (I) or a salt thereof, or compound (II)) is solubilized or diluted in a buffer comprising 0.1-3.0, 0.2-3.0, 0.3-3.0, 0.4-3.0, 0.5-3.0, 0.6-3.0, 0.7-3.0, 0.8-3.0, 0.9-3.0, 1.0-3.0, 1.2-3.0, 1.3-3.0, 1.4-3.0, 1.5-3.0, 1.6-3.0, 1.7-3.0, 1.8-3.0, 1.9-3.0, 2.0-3.0, 2.1-3.0, 2.2-3.0, 2.3-3.0, 2.4-3.0, 2.5-3.0, 2.6-3.0, 2.7-3.0, 2.8-3.0, or 2.9-3.0 mM NaH₂PO₄. In some embodiments, the compound as described herein (e.g., compound (I) or a salt thereof, or compound (II)) is solubilized or diluted in a buffer comprising at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM NaH₂PO₄. In some embodiments, the compound as described herein (e.g., compound (I) or a salt thereof, or compound (II)) is solubilized or diluted in a buffer comprising at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM NaH₂PO₄. In some embodiments, the compound as described herein (e.g., compound (I) or a salt thereof, or compound (II)) is solubilized or diluted in a buffer comprising 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM NaH₂PO₄.

In some embodiments, the compound as described herein (e.g., compound (I) or a salt thereof, or compound (II)) is solubilized or diluted in a buffer comprising 0.1-50 mM CaCl₂.

In some embodiments, the compound as described herein (e.g., compound (I) or a salt thereof, or compound (II)) is solubilized or diluted in a buffer comprising 0.1-50, 0.2-50, 0.3-50, 0.4-50, 0.5-50, 0.6-50, 0.7-50, 0.8-50, 0.9-50, 1.0-50, −50, 1.1-50, 1.2-50, 1.3-50, 1.4-50, 1.5-50, 1.6-50, 1.7-50, 1.8-50, 1.9-50, 2.0-50, 2.1-50, 2.2-50, 2.3-50, 2.4-50, 2.5-50, 2.6-50, 2.7-50, 2.8-50, 2.9-50, 3.0-50, 3.1-50, 3.2-50, 3.3-50, 3.4-50, 3.5-50, 3.6-50, 3.7-50, 3.8-50, 3.9-50, 4-50, 5-50, 6-50, 7-50, 8-50, 9-50, 10-50, 15-50, 20-50, 25-50, 1 30-50, 35-50, 40-50, or 45-50 mM CaCl₂. In some embodiments, the compound as described herein (e.g., compound (I) or a salt thereof, or compound (II)) is solubilized or diluted in a buffer comprising 0.1-45, 0.1-40, 0.1-35, 0.1-30, 0.1-25, 0.1-20, 0.1-15, 0.1-10, 0.1-5, 0.1-4, 0.1-4.9, 0.1-4.8, 0.1-4.7, 0.1-4.6, 0.1-4.5, 0.1-4.4, 0.1-4.3, 0.1-4.2, 0.1-4.1, 0.1-4.0, 0.1-3.9, 0.1-3.8, 0.1-3.7, 0.1-3.6, 0.1-3.5, 0.1-3.4, 0.1-3.3, 0.1-3.2, 0.1-3.1, 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM CaCl₂. In some embodiments, the compound as described herein (e.g., compound (I) or a salt thereof, or compound (II)) is solubilized or diluted in a buffer comprising at least 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM CaCl₂. In some embodiments, the compound as described herein (e.g., compound (I) or a salt thereof, or compound (II)) is solubilized or diluted in a buffer comprising at most 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM CaCl₂. In some embodiments, the compound as described herein (e.g., compound (I) or a salt thereof, or compound (II)) is solubilized or diluted in a buffer comprising 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM CaCl₂.

In some embodiments, the compound as described herein (e.g., compound (I) or a salt thereof, or compound (II)) is solubilized or diluted in a buffer comprising 0.1-50 mM MgCl₂.

In some embodiments, the compound as described herein (e.g., compound (I) or a salt thereof, or compound (II)) is solubilized or diluted in a buffer comprising 0.1-50, 0.2-50, 0.3-50, 0.4-50, 0.5-50, 0.6-50, 0.7-50, 0.8-50, 0.9-50, 1.0-50, −50, 1.1-50, 1.2-50, 1.3-50, 1.4-50, 1.5-50, 1.6-50, 1.7-50, 1.8-50, 1.9-50, 2.0-50, 2.1-50, 2.2-50, 2.3-50, 2.4-50, 2.5-50, 2.6-50, 2.7-50, 2.8-50, 2.9-50, 3.0-50, 3.1-50, 3.2-50, 3.3-50, 3.4-50, 3.5-50, 3.6-50, 3.7-50, 3.8-50, 3.9-50, 4-50, 5-50, 6-50, 7-50, 8-50, 9-50, 10-50, 15-50, 20-50, 25-50, 1 30-50, 35-50, 40-50, or 45-50 mM MgCl₂. In some embodiments, the compound as described herein (e.g., compound (I) or a salt thereof, or compound (II)) is solubilized or diluted in a buffer comprising 0.1-45, 0.1-40, 0.1-35, 0.1-30, 0.1-25, 0.1-20, 0.1-15, 0.1-10, 0.1-5, 0.1-4, 0.1-4.9, 0.1-4.8, 0.1-4.7, 0.1-4.6, 0.1-4.5, 0.1-4.4, 0.1-4.3, 0.1-4.2, 0.1-4.1, 0.1-4.0, 0.1-3.9, 0.1-3.8, 0.1-3.7, 0.1-3.6, 0.1-3.5, 0.1-3.4, 0.1-3.3, 0.1-3.2, 0.1-3.1, 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM MgCl₂. In some embodiments, the compound as described herein (e.g., compound (I) or a salt thereof, or compound (II)) is solubilized or diluted in a buffer comprising at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM MgCl₂. In some embodiments, the compound as described herein (e.g., compound (I) or a salt thereof, or compound (II)) is solubilized or diluted in a buffer comprising at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM MgCl₂. In some embodiments, the compound as described herein (e.g., compound (I) or a salt thereof, or compound (II)) is solubilized or diluted in a buffer comprising 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM MgCl₂.

In some embodiments, the compound as described herein (e.g., compound (I) or a salt thereof, or compound (II)) is solubilized or diluted in a buffer further comprising 1-100 mM NaHCO₃, 1-100 mM KHCO₃, or a combination thereof.

In some embodiments, the compound as described herein (e.g., compound (I) or a salt thereof, or compound (II)) is solubilized or diluted in a buffer comprising 1-100 mM NaHCO₃.

In some embodiments, the compound as described herein (e.g., compound (I) or a salt thereof, or compound (II)) is solubilized or diluted in a buffer comprising 1-99, 1-95, 1-90, 1-85, 1-80, 1-75, 1-70, 1-65, 1-60, 1-55, 1-50, 1-45, 1-40, 1-35, 1-30, 1-25, 1-20, 1-15, 1-10, 1-9, 1-8, 1-7, 1-6, 1-5, 1-4, 1-3, or 1-2 mM NaHCO₃. In some embodiments, the compound as described herein (e.g., compound (I) or a salt thereof, or compound (II)) is solubilized or diluted in a buffer comprising 2-100, 3-100, 4-100, 5-100, 6-100, 7-100, 8-100, 9-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100 mM NaHCO₃. In some embodiments, the compound as described herein (e.g., compound (I) or a salt thereof, or compound (II)) is solubilized or diluted in a buffer comprising 24.0-28.0, 24.0-27.9, 24.0-27.8, 24.0-27.7, 24.0-27.6, 24.0-27.5, 24.0-27.4, 24.0-27.3, 24.0-27.2, 24.0-27.1, 24.0-27.0, 24.0-26.9, 24.0-26.8, 24.0-26.7, 24.0-26.6, 24.0-26.5, 24.0-26.4, 24.0-26.3, 24.0-26.2, 24.0-26.1, 24.0-26.0, 24.0-25.9, 4.0-25.8, 24.0-25.7, 24.0-25.6, 24.0-25.5, 24.0-25.4, 24.0-25.3, 24.0-25.2, 24.0-25.1, 24.0-25.0, 24.0-24.9, 24.0-24.8, 24.0-24.7, 24.0-24.6, 24.0-24.5, 24.0-24.4, 24.0-24.3, 24.0-24.2, or 24.0-24.1 mM NaHCO₃. In some embodiments, the compound as described herein (e.g., compound (I) or a salt thereof, or compound (II)) is solubilized or diluted in a buffer comprising 24.1-28.0, 24.2-28.0, 24.3-28.0, 24.4-28.0, 24.5-28.0, 24.6-28.0, 24.7-28.0, 24.8-28.0, 24.9-28.0, 25.0-28.0, 25.1-28.0, 25.2-28.0, 25.3-28.0, 25.4-28.0, 25.5-28.0, 25.6-28.0, 25.7-28.0, 25.8-28.0, 25.9-28.0, 26.0-28.0, 26.1-28.0, 26.2-28.0, 26.3-28.0, 26.4-28.0, 26.5-28.0, 26.6-28.0, 26.7-28.0, 26.8-28.0, 26.9-28.0, 27.0-28.0, 27.1-28.0, 27.2-28.0, 27.3-28.0, 27.4-28.0, 27.5-28.0, 27.6-28.0, 27.7-28.0, 27.8-28.0, or 27.9-28.0 mM NaHCO₃. In some embodiments, the compound as described herein (e.g., compound (I) or a salt thereof, or compound (II)) is solubilized or diluted in a buffer comprising at least 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM NaHCO₃. In some embodiments, the compound as described herein (e.g., compound (I) or a salt thereof, or compound (II)) is solubilized or diluted in a buffer comprising at most 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM NaHCO₃. In some embodiments, the compound as described herein (e.g., compound (I) or a salt thereof, or compound (II)) is solubilized or diluted in a buffer comprising 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM NaHCO₃.

In some embodiments, the compound as described herein (e.g., compound (I) or a salt thereof, or compound (II)) is solubilized or diluted in a buffer comprising 1-100 mM KHCO₃.

In some embodiments, the compound as described herein (e.g., compound (I) or a salt thereof, or compound (II)) is solubilized or diluted in a buffer comprising 1-99, 1-95, 1-90, 1-85, 1-80, 1-75, 1-70, 1-65, 1-60, 1-55, 1-50, 1-45, 1-40, 1-35, 1-30, 1-25, 1-20, 1-15, 1-10, 1-9, 1-8, 1-7, 1-6, 1-5, 1-4, 1-3, or 1-2 mM KHCO₃. In some embodiments, the compound as described herein (e.g., compound (I) or a salt thereof, or compound (II)) is solubilized or diluted in a buffer comprising 2-100, 3-100, 4-100, 5-100, 6-100, 7-100, 8-100, 9-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100 mM KHCO₃. In some embodiments, the compound as described herein (e.g., compound (I) or a salt thereof, or compound (II)) is solubilized or diluted in a buffer comprising 24.0-28.0, 24.0-27.9, 24.0-27.8, 24.0-27.7, 24.0-27.6, 24.0-27.5, 24.0-27.4, 24.0-27.3, 24.0-27.2, 24.0-27.1, 24.0-27.0, 24.0-26.9, 24.0-26.8, 24.0-26.7, 24.0-26.6, 24.0-26.5, 24.0-26.4, 24.0-26.3, 24.0-26.2, 24.0-26.1, 24.0-26.0, 24.0-25.9, 4.0-25.8, 24.0-25.7, 24.0-25.6, 24.0-25.5, 24.0-25.4, 24.0-25.3, 24.0-25.2, 24.0-25.1, 24.0-25.0, 24.0-24.9, 24.0-24.8, 24.0-24.7, 24.0-24.6, 24.0-24.5, 24.0-24.4, 24.0-24.3, 24.0-24.2, or 24.0-24.1 mM KHCO₃. In some embodiments, the compound as described herein (e.g., compound (I) or a salt thereof, or compound (II)) is solubilized or diluted in a buffer comprising 24.1-28.0, 24.2-28.0, 24.3-28.0, 24.4-28.0, 24.5-28.0, 24.6-28.0, 24.7-28.0, 24.8-28.0, 24.9-28.0, 25.0-28.0, 25.1-28.0, 25.2-28.0, 25.3-28.0, 25.4-28.0, 25.5-28.0, 25.6-28.0, 25.7-28.0, 25.8-28.0, 25.9-28.0, 26.0-28.0, 26.1-28.0, 26.2-28.0, 26.3-28.0, 26.4-28.0, 26.5-28.0, 26.6-28.0, 26.7-28.0, 26.8-28.0, 26.9-28.0, 27.0-28.0, 27.1-28.0, 27.2-28.0, 27.3-28.0, 27.4-28.0, 27.5-28.0, 27.6-28.0, 27.7-28.0, 27.8-28.0, or 27.9-28.0 mM KHCO₃. In some embodiments, the compound as described herein (e.g., compound (I) or a salt thereof, or compound (II)) is solubilized or diluted in a buffer comprising at least 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM KHCO₃. In some embodiments, the compound as described herein (e.g., compound (I) or a salt thereof, or compound (II)) is solubilized or diluted in a buffer comprising at most 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM KHCO₃. In some embodiments, the compound as described herein (e.g., compound (I) or a salt thereof, or compound (II)) is solubilized or diluted in a buffer comprising 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM KHCO₃.

In some embodiments, the compound as described herein (e.g., compound (I) or a salt thereof, or compound (II)) is solubilized or diluted in a buffer comprising 0-50 mM KH₂PO₄.

In some embodiments, the compound as described herein (e.g., compound (I) or a salt thereof, or compound (II)) is solubilized or diluted in a buffer comprising 0-100, 0.01-100, 0.02-100, 0.03-100, 0.04-100, 0.05-100, 0.06-100, 0.07-100, 0.08-100, 0.09-100, 0.1-100, 0.2-100, 0.3-100, 0.4-100, 0.5-100, 0.6-100, 0.7-100, 0.8-100, 0.9-100, 1-100, 2-100, 3-100, 4-100, 5-100, 6-100, 7-100, 8-100, 9-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100 mM KH₂PO₄. In some embodiments, the compound as described herein (e.g., compound (I) or a salt thereof, or compound (II)) is solubilized or diluted in a buffer comprising 0-95, 0-90, 0-85, 0-80, 0-75, 0-70, 0-65, 0-60, 0-55, 0-50, 0-45, 0-40, 0-35, 0-30, 0-25, 0-20, 0-15, 0-10, 0-9, 0-8, 0-7, 0-6, 0-5, 0-4, 0-3, 0-2, 0-1, 0-0.9, 0-0.8, 0-0.7, 0-0.6, 0-0.5, 0-0.4, 0-0.3, 0-0.2, 0-0.1, 0-0.09, 0-0.08, 0-0.07, 0-0.06, 0-0.05, 0-0.04, 0-0.03, or 0-0.02 mM KH₂PO₄. In some embodiments, the compound as described herein (e.g., compound (I) or a salt thereof, or compound (II)) is solubilized or diluted in a buffer comprising 0.01-95, 0.01-90, 0.01-85, 0.01-80, 0.01-75, 0.01-70, 0.01-65, 0.01-60, 0.01-55, 0.01-50, 0.01-45, 0.01-40, 0.01-35, 0.01-30, 0.01-25, 0.01-20, 0.01-15, 0.01-10, 0.01-9, 0.01-8, 0.01-7, 0.01-6, 0.01-5, 0.01-4, 0.01-3, 0.01-2, 0.01-1, 0.01-0.9, 0.01-0.8, 0.01-0.7, 0.01-0.6, 0.01-0.5, 0.01-0.4, 0.01-0.3, 0.01-0.2, 0.01-0.1, 0.01-0.09, 0.01-0.08, 0.01-0.07, 0.01-0.06, 0.01-0.05, 0.01-0.04, 0.01-0.03, or 0.01-0.02 mM KH₂PO₄. In some embodiments, the compound as described herein (e.g., compound (I) or a salt thereof, or compound (II)) is solubilized or diluted in a buffer comprising 0-3.0, 0-2.9, 0-2.8, 0-2.7, 0-2.6, 0-2.5, 0-2.4, 0-2.3, 0-2.2, 0-2.1, 0-2.0, 0-1.9, 0-1.8, 0-1.7, 0-1.6, 0-1.5, 0-1.4, 0-1.3, 0-1.2, 0-1.1, 0-1.0, 0-0.9, 0-0.8, 0-0.7, 0-0.6, 0-0.5, 0-0.4, 0-0.3, or 0-0.2 mM KH₂PO₄. In some embodiments, the compound as described herein (e.g., compound (I) or a salt thereof, or compound (II)) is solubilized or diluted in a buffer comprising 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM KH₂PO₄. In some embodiments, the compound as described herein (e.g., compound (I) or a salt thereof, or compound (II)) is solubilized or diluted in a buffer comprising 0-3.0, 0.1-3.0, 0.2-3.0, 0.3-3.0, 0.4-3.0, 0.5-3.0, 0.6-3.0, 0.7-3.0, 0.8-3.0, 0.9-3.0, 1.0-3.0, 1.2-3.0, 1.3-3.0, 1.4-3.0, 1.5-3.0, 1.6-3.0, 1.7-3.0, 1.8-3.0, 1.9-3.0, 2.0-3.0, 2.1-3.0, 2.2-3.0, 2.3-3.0, 2.4-3.0, 2.5-3.0, 2.6-3.0, 2.7-3.0, 2.8-3.0, or 2.9-3.0 mM KH₂PO₄. In some embodiments, the compound as described herein (e.g., compound (I) or a salt thereof, or compound (II)) is solubilized or diluted in a buffer comprising at least 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM KH₂PO₄. In some embodiments, the compound as described herein (e.g., compound (I) or a salt thereof, or compound (II)) is solubilized or diluted in a buffer comprising at most 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM KH₂PO₄. In some embodiments, the compound as described herein (e.g., compound (I) or a salt thereof, or compound (II)) is solubilized or diluted in a buffer comprising 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mM KH₂PO₄.

In some embodiments, the compound as described herein (e.g., compound (I) or a salt thereof, or compound (II)) is solubilized or diluted in a buffer comprising 0-50 mM NaH₂PO₄.

In some embodiments, the compound as described herein (e.g., compound (I) or a salt thereof, or compound (II)) is solubilized or diluted in a buffer comprising 0-50, 0-45, 0-40, 0-35, 0-30, 0-25, 0-20, 0-19, 0-18, 0-17, 0-16, 0-15, 0-14, 0-13, 0-12, 0-11, 0-10, 0-9, 0-8, 0-7, 0-6, 0-5, 0-4, 0-3, 0-2, 0-1, 0-0.9, 0-0.8, 0-0.7, 0-0.6, 0-0.5, 0-0.4, 0-0.3, or 0-0.2 mM NaH₂PO₄. In some embodiments, the compound as described herein (e.g., compound (I) or a salt thereof, or compound (II)) is solubilized or diluted in a buffer comprising 0.1-50, 0.1-45, 0.1-40, 0.1-35, 0.1-30, 0.1-25, 0.1-20, 0.1-19, 0.1-18, 0.1-17, 0.1-16, 0.1-15, 0.1-14, 0.1-13, 0.1-12, 0.1-11, 0.1-10, 0.1-9, 0.1-8, 0.1-7, 0.1-6, 0.1-5, 0.1-4, 0.1-3, 0.1-2, 0.1-1, 0.1-0.9, 0.1-0.8, 0.1-0.7, 0.1-0.6, 0.1-0.5, 0.1-0.4, 0.1-0.3, or 0.1-0.2 mM NaH₂PO₄. In some embodiments, the compound as described herein (e.g., compound (I) or a salt thereof, or compound (II)) is solubilized or diluted in a buffer comprising 0-50, 0.1-50, 0.2-50, 0.3-50, 0.4-50, 0.5-50, 0.6-50, 0.7-50, 0.8-50, 0.9-50, 1-50, 2-50, 3-50, 4-50, 5-50, 6-50, 7-50, 8-50, 9-50, 1-50, 11-50, 12-50, 13-50, 14-50, 15-50, 16-50, 17-50, 18-50, 19-50, 20-50, 25-50, 30-50, 35-50, 40-50, or 45-50 mM NaH₂PO₄. In some embodiments, the compound as described herein (e.g., compound (I) or a salt thereof, or compound (II)) is solubilized or diluted in a buffer comprising 0-20, 0.1-20, 0.2-20, 0.3-20, 0.4-20, 0.5-20, 0.6-20, 0.7-20, 0.8-20, 0.9-20, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, or 19-20 mM NaH₂PO₄. In some embodiments, the compound as described herein (e.g., (I) or a salt thereof, or compound (II)) is solubilized or diluted in a buffer comprising at least 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mM NaH₂PO₄. In some embodiments, the compound as described herein (e.g., compound (I) or a salt thereof, or compound (II)) is solubilized or diluted in a buffer comprising at most 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mM NaH₂PO₄. In some embodiments, the compound as described herein (e.g., compound (I) or a salt thereof, or compound (II)) is solubilized or diluted in a buffer comprising 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mM NaH₂PO₄.

In some embodiments, the compound as described herein (e.g., compound (I) or a salt thereof, or compound (II)) is solubilized or diluted in a buffer further comprising carbohydrates. In some embodiments, the carbohydrates comprise D-glucose. In some embodiments, the compound as described herein (e.g., compound (I) or a salt thereof, or compound (II)) is solubilized or diluted in a buffer further comprising 1-100 mM D-glucose.

In some embodiments, the compound as described herein (e.g., compound (I) or a salt thereof, or compound (II)) is solubilized or diluted in a buffer comprising 1-100 mM D-glucose.

In some embodiments, the compound as described herein (e.g., compound (I) or a salt thereof, or compound (II)) is solubilized or diluted in a buffer comprising 1-100, 1-95, 1-90, 1-85, 1-80, 1-75, 1-70, 1-65, 1-60, 1-55, 1-50, 1-45, 1-40, 1-35, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 mM D-glucose. In some embodiments, the compound as described herein (e.g., compound (I) or a salt thereof, or compound (II)) is solubilized or diluted in a buffer comprising 2-100, 3-100, 4-100, 5-100, 6-100, 7-100, 8-100, 9-100, 10-100, 11-100, 12-100, 13-100, 14-100, 15-100, 16-100, 17-100, 18-100, 19-100, 20-100, 21-100, 22-100, 23-100, 24-100, 25-100, 26-100, 29-100, 28-100, 29-100, 30-100, 35-100, 40-100, 45-100, 50-100, 55-100, 60-100, 65-100, 70-100, 75-100, 80-100, 85-100, 90-100, or 95-100 mM D-glucose. In some embodiments, the compound as described herein (e.g., compound (I) or a salt thereof, or compound (II)) is solubilized or diluted in a buffer comprising 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 mM D-glucose. In some embodiments, the compound as described herein (e.g., compound (I) or a salt thereof, or compound (II)) is solubilized or diluted in a buffer comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM D-glucose. In some embodiments, the compound as described herein (e.g., compound (I) or a salt thereof, or compound (II)) is solubilized or diluted in a buffer comprising at most 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM D-glucose. In some embodiments, the compound as described herein (e.g., compound (I) or a salt thereof, or compound (II)) is solubilized or diluted in a buffer comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM D-glucose.

In some embodiments, the compound as described herein (e.g., compound (I) or a salt thereof, or compound (II)) is solubilized or diluted in a buffer comprising 25-250 mM NaCl, 0.1-20 mM KCl, 0.1-50 mM Na₂HPO₄, 0.1-50 mM NaH₂PO₄, 0.1-50 mM CaCl₂, and 0.1-50 mM MgCl₂.

In some embodiments, the compound as described herein (e.g., compound (I) or a salt thereof, or compound (II)) is solubilized or diluted in a buffer comprising 150 mM NaCl, 3.0 mM KCl, 0.7 mM Na₂HPO₄, 0.3 mM NaH₂PO₄, 0.79 mM MgCl₂, and 1.4 mM CaCl₂.

In some embodiments, the compound as described herein (e.g., compound (I) or a salt thereof, or compound (II)) is solubilized or diluted in a buffer further comprising an antioxidant. In some embodiments, the antioxidant is t-butylhydroxyquinoline (TBHQ), butylated hydroxytoluene (BHT), butylated hydroxyanisole (BHA), vitamin E, or any combination thereof. In some embodiments, the compound as described herein (e.g., compound (I) or a salt thereof, or compound (II)) is solubilized or diluted in a buffer further comprising an antioxidant, wherein the antioxidant is ascorbic acid (vitamin C), glutathione, lipoic acid, uric acid, carotenes, α-tocopherol (vitamin E), ubiquinol (coenzyme Q), or any combination thereof.

In some embodiments, the compound as described herein (e.g., compound (I) or a salt thereof, or compound (II)) is solubilized or diluted in a buffer comprising 25-250 mM NaCl, 0.1-20 mM KCl, 0-50 mM KH₂PO₄, 1-100 mM NaHCO₃, 0-50 mM NaH₂PO₄, 1-100 mM D-glucose, 0.1-50 mM CaCl₂, 0.1-50 mM MgCl₂, or any combination thereof.

In some embodiments, the compound as described herein (e.g., compound (I) or a salt thereof, or compound (II)) is solubilized or diluted in a buffer comprising 25-250 mM NaCl, 0.1-20 mM KCl, 0-50 mM KH₂PO₄, 1-100 mM NaHCO₃, 0-50 mM NaH₂PO₄, 1-100 mM D-glucose, 0.1-50 mM CaCl₂, and 0.1-50 mM MgCl₂.

In some embodiments, the compound as described herein (e.g., compound (I) or a salt thereof, or compound (II)) is solubilized or diluted in a buffer comprising 127 mM NaCl, 1.0 mM KCl, 1.2 mM KH₂PO₄, 26 mM NaHCO₃, 10 mM D-glucose, 2.4 mM CaCl₂, and 1.3 mM MgCl₂.

In some embodiments, the compound as described herein (e.g., compound (I) or a salt thereof, or compound (II)) is solubilized or diluted in a buffer comprising 119 mM NaCl, 26.2 mM NaHCO₃, 2.5 mM KCl, 1 mM NaH₂PO₄, 1.3 mM MgCl₂, 10 mM glucose, and 2.5 mM CaCl₂.

In some embodiments, the pharmaceutical formulation does not comprise a preservative. In some embodiments, the pharmaceutical formulation comprises a preservative.

In some embodiments, the compound as described herein (e.g., compound (I) or a salt thereof, or compound (II)) is solubilized or diluted to a concentration of from 5-250 mg/mL in the diluent.

In some embodiments, the compound as described herein (e.g., compound (I) or a salt thereof, or compound (II)) is solubilized or diluted to a concentration of from 5-250, 5-247.5, 5-245, 5-242.5, 5-240, 5-237.5, 5-235, 5-232.5, 5-230, 5-227.5, 5-225, 5-225.5, 5-220, 5-217.5, 5-215, 5-212.5, 5-210, 5-205.5, 5-205, 5-202.5, 5-200, 5-197.5, 5-195, 5-192.5, 5-190, 5-187.5, 5-185, 5-182.5, 5-180, 5-177.5, 5-175, 5-172.5, 5-170, 5-167.5, 5-165, 5-162.5, 5-160, 5-157.5, 5-155, 5-152.5, 5-150, 5-147.5, 5-145, 5-142.5, 5-140, 5-137.5, 5-135, 5-132.5, 5-130, 5-127.5, 5-125, 5-122.5, 5-120, 5-117.5, 5-115, 5-112.5, 5-110, 5-107.5, 5-105, 5-102.5, 5-100, 5-97.5, 5-95, 5-92.5, 5-90, 5-87.5, 5-85, 5-82.5, 5-80, 5-77.5, 5-75, 5-72.5, 5-70, 5-67.5, 5-65, 5-62.5, 5-60, 5-57.5, 5-55, 5-52.5, 5-50, 5-47.5, 5-45, 5-42.5, 5-40, 5-37.5, 5-35, 5-32.5, 5-30, 5-27.5, 5-25, 5-22.5, 5-20, 5-17.5, 5-15, 5-12.5, or 5-10 mg/mL in the diluent. In some embodiments, the compound as described herein (e.g., compound (I) or a salt thereof, or compound (II)) is solubilized or diluted to a concentration of from 10-250, 15-250, 20-250, 25-250, 30-250, 35-250, 40-250, 45-250, 50-250, 55-250, 60-250, 65-250, 70-250, 75-250, 80-250, 85-250, 90-250, 95-250, 100-250, 105-250, 110-250, 115-250, 120-250, 125-250, 130-250, 135-250, 140-250, 145-250, 150-250, 155-250, 160-250, 165-250, 170-250, 175-250, 180-250, 185-250, 190-250, or 195-250, 200-250, 205-250, 210-250, 215-250, 220-250, 225-250, 230-250, 235-250, 240-250, or 245-250 mg/mL in the diluent. In some embodiments, the compound as described herein (e.g., compound (I) or a salt thereof, or compound (II)) is solubilized or diluted to a concentration of from at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent. In some embodiments, the compound as described herein (e.g., compound (I) or a salt thereof, or compound (II)) is solubilized or diluted to a concentration of from at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent. In some embodiments, the compound as described herein (e.g., compound (I) or a salt thereof, or compound (II)) is solubilized or diluted to a concentration of from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent.

In some embodiments, the compound as described herein (e.g., compound (I) or a salt thereof, or compound (II)) is present in the pharmaceutical composition at a concentration of the compound as described herein (e.g., compound (I) or a salt thereof, or compound (II)) is present in the pharmaceutical composition at a concentration of from 0.1 mg/mL to 250 mg/mL. In some embodiments, the compound as described herein (e.g., compound (I) or a salt thereof, or compound (II)) is present in the pharmaceutical composition at a concentration of about 0.1 mg/mL, 0.5 mg/mL, 1 mg/mL, 2 mg/mL, 2.5 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, or 20 mg/mL. In some embodiments, the compound as described herein (e.g., compound (I) or a salt thereof, or compound (II)) is present in the pharmaceutical composition at a concentration of about 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL. In some embodiments, the compound as described herein (e.g., compound (I) or a salt thereof, or compound (II)) is present in the pharmaceutical composition at a concentration of about 22.5 mg/mL, 25 mg/mL, 27.5 mg/mL, 30 mg/mL, 32.5 mg/mL, 35 mg/mL, 37.5 mg/mL, 40 mg/mL, 42.5 mg/mL, 45 mg/mL, 47.5 mg/mL, 50 mg/mL, 52.5 mg/mL, 55 mg/mL, 57.5 mg/mL, 60 mg/mL, 62.5 mg/mL, 65 mg/mL, 67.5 mg/mL, 70 mg/mL, 72.5 mg/mL, 75 mg/mL, 77.5 mg/mL, 80 mg/mL, 82.5 mg/mL, 85 mg/mL, 87.5 mg/mL, 90 mg/mL, 92.5 mg/mL, 95 mg/mL, 97.5 mg/mL, 100 mg/mL, 102.5 mg/mL, 105 mg/mL, 107.5 mg/mL, 110 mg/mL, 112.5 mg/mL, 115 mg/mL, 117.5 mg/mL, 120 mg/mL, 122.5 mg/mL, 125 mg/mL, 127.5 mg/mL, 130 mg/mL, 132.5 mg/mL, 135 mg/mL, 137.5 mg/mL, 140 mg/mL, 142.5 mg/mL, 145 mg/mL, 147.5 mg/mL, 150 mg/mL, 152.5 mg/mL, 155 mg/mL, 157.5 mg/mL, 160 mg/mL, 162.5 mg/mL, 165 mg/mL, 167.5 mg/mL, 170 mg/mL, 172.5 mg/mL, 175 mg/mL, 177.5 mg/mL, 180 mg/mL, 182.5 mg/mL, 185 mg/mL, 187.5 mg/mL, 190 mg/mL, 192.5 mg/mL, 195 mg/mL, 197.5 mg/mL, 200 mg/mL, 202.5 mg/mL, 205 mg/mL, 207.5 mg/mL, 210 mg/mL, 212.5 mg/mL, 215 mg/mL, 217.5 mg/mL, 220 mg/mL, 222.5 mg/mL, 225 mg/mL, 227.5 mg/mL, 230 mg/mL, 232.5 mg/mL, 235 mg/mL, 237.5 mg/mL, 240 mg/mL, 242.5 mg/mL, 245 mg/mL, 247.5 mg/mL, or 250 mg/mL. In some embodiments, the compound as described herein (e.g., compound (I) or a salt thereof, or compound (II)) is solubilized or diluted to a concentration of 11 mg/mL, 22 mg/mL, 33 mg/mL, 44 mg/mL, 55 mg/mL, 66 mg/mL, 77 mg/mL, 88 mg/mL, 99 mg/mL, or 100 mg/mL in the diluent.

In some embodiments, the compound as described herein (e.g., compound (I) or a salt thereof, or compound (II)) is present in the pharmaceutical composition at a concentration of from 0.1 mg/mL to 250 mg/mL, from 0.2 mg/mL to 250 mg/mL, from 0.3 mg/mL to 250 mg/mL, from 0.4 mg/mL to 250 mg/mL, from 0.5 mg/mL to 250 mg/mL, from 0.6 mg/mL to 250 mg/mL, from 0.7 mg/mL to 250 mg/mL, from 0.8 mg/mL to 250 mg/mL, from 0.9 mg/ml to 250 mg/mL, from 1.0 mg/mL to 250 mg/mL, from 1.1 mg/mL to 250 mg/mL, from 1.2 mg/mL to 250 mg/mL, from 1.3 mg/mL to 250 mg/mL, from 1.4 mg/mL to 250 mg/mL, from 1.5 mg/mL to 250 mg/mL, from 1.6 mg/mL to 250 mg/mL, from 1.7 mg/mL to 250 mg/mL, from 1.8 mg/mL to 250 mg/mL, from 1.9 mg/mL to 250 mg/mL, from 2.0 mg/mL to 250 mg/mL, from 2.1 mg/mL to 250 mg/mL, from 2.2 mg/mL to 250 mg/mL, from 2.3 mg/mL to 250 mg/mL, from 2.4 mg/mL to 250 mg/mL, from 2.5 mg/mL to 250 mg/mL, from 2.6 mg/mL to 250 mg/mL, from 2.7 mg/mL to 250 mg/mL, from 2.8 mg/mL to 250 mg/mL, from 2.9 mg/mL to 250 mg/mL, from 3.0 mg/mL to 250 mg/mL, from 3.1 mg/mL to 250 mg/mL, from 3.2 mg/mL to 250 mg/mL, from 3.3 mg/mL to 250 mg/mL, from 3.4 mg/mL to 250 mg/mL, from 3.5 mg/mL to 250 mg/mL, from 3.6 mg/mL to 250 mg/mL, from 3.7 mg/mL to 250 mg/mL, from 3.8 mg/mL to 250 mg/mL, from 3.9 mg/mL to 250 mg/mL, from 4.0 mg/mL to 250 mg/mL, from 5.0 mg/mL to 250 mg/mL, from 6.0 mg/mL to 250 mg/mL, from 7.0 mg/mL to 250 mg/mL, from 8.0 mg/mL to 250 mg/mL, from 9.0 mg/mL to 250 mg/mL, from 10 mg/mL to 250 mg/mL, from 15 mg/mL to 250 mg/mL, from 20 mg/mL to 250 mg/mL, from 25 mg/mL to 250 mg/mL, from 30 mg/mL to 250 mg/mL, from 35 mg/mL to 250 mg/mL, from 40 mg/mL to 250 mg/mL, from 45 mg/mL to 250 mg/mL, from 50 mg/mL to 250 mg/mL, from 55 mg/mL to 250 mg/mL, from 60 mg/mL to 250 mg/mL, from 65 mg/mL to 250 mg/mL, from 70 mg/mL to 250 mg/mL, from 75 mg/mL to 250 mg/mL, from 80 mg/mL to 250 mg/mL, from 85 mg/mL to 250 mg/mL, from 90 mg/mL to 250 mg/mL, from 95 mg/mL to 250 mg/mL, from 100 mg/mL to 250 mg/mL, from 105 mg/mL to 250 mg/mL, from 110 mg/mL to 250 mg/mL, from 115 mg/mL to 250 mg/mL, from 120 mg/mL to 250 mg/mL, from 125 mg/mL to 250 mg/mL, from 130 mg/mL to 250 mg/mL, from 135 mg/mL to 250 mg/mL, from 140 mg/mL to 250 mg/mL, from 145 mg/mL to 250 mg/mL, from 150 mg/mL to 250 mg/mL, from 155 mg/mL to 250 mg/mL, from 160 mg/mL to 250 mg/mL, from 165 mg/mL to 250 mg/mL, from 170 mg/mL to 250 mg/mL, from 175 mg/mL to 250 mg/mL, from 180 mg/mL to 250 mg/mL, from 185 mg/mL to 250 mg/mL, from 190 mg/mL to 250 mg/mL, from 195 mg/mL to 250 mg/mL, 200 mg/mL to 250 mg/mL, from 205 mg/mL to 250 mg/mL, from 210 mg/mL to 250 mg/mL, from 215 mg/mL to 250 mg/mL, from 220 mg/mL to 250 mg/mL, from 225 mg/mL to 250 mg/mL, from 230 mg/mL to 250 mg/mL, from 235 mg/mL to 250 mg/mL, from 240 mg/mL to 250 mg/mL, or from 245 mg/mL to 250 mg/mL.

In some embodiments, the compound as described herein (e.g., compound (I) or a salt thereof, or compound (II)) is present in the pharmaceutical composition at a concentration of from 0.1 mg/mL to 250 mg/mL, from 0.1 mg/mL to 245 mg/mL, from 0.1 mg/mL to 240 mg/mL, from 0.1 mg/mL to 235 mg/mL, from 0.1 mg/mL to 230 mg/mL, from 0.1 mg/mL to 225 mg/mL, from 0.1 mg/mL to 220 mg/mL, from 0.1 mg/mL to 215 mg/mL, from 0.1 mg/mL to 210 mg/mL, from 0.1 mg/mL to 205 mg/mL, from 0.1 mg/mL to 200 mg/mL, from 0.1 mg/mL to 195 mg/mL, from 0.1 mg/mL to 190 mg/mL, from 0.1 mg/mL to 185 mg/mL, from 0.1 mg/mL to 180 mg/mL, from 0.1 mg/mL to 175 mg/mL, from 0.1 mg/mL to 170 mg/mL, from 0.1 mg/mL to 165 mg/mL, from 0.1 mg/mL to 160 mg/mL, from 0.1 mg/mL to 155 mg/mL, from 0.1 mg/mL to 150 mg/mL, from 0.1 mg/mL to 145 mg/mL, from 0.1 mg/mL to 140 mg/mL, from 0.1 mg/mL to 135 mg/mL, from 0.1 mg/mL to 130 mg/mL, from 0.1 mg/mL to 125 mg/mL, from 0.1 mg/mL to 120 mg/mL, from 0.1 mg/mL to 115 mg/mL, from 0.1 mg/mL to 110 mg/mL, from 0.1 mg/mL to 100 mg/mL, from 0.1 mg/mL to 95 mg/mL, from 0.1 mg/mL to 90 mg/mL, from 0.1 mg/mL to 85 mg/mL, from 0.1 mg/mL to 80 mg/mL, from 0.1 mg/mL to 75 mg/mL, from 0.1 mg/mL to 70 mg/mL, from 0.1 mg/mL to 65 mg/mL, from 0.1 mg/mL to 60 mg/mL, from 0.1 mg/mL to 55 mg/mL, from 0.1 mg/mL to 50 mg/mL, from 0.1 mg/mL to 45 mg/mL, from 0.1 mg/mL to 40 mg/mL, from 0.1 mg/mL to 35 mg/mL, from 0.1 mg/mL to 30 mg/mL, from 0.1 mg/mL to 25 mg/mL, from 0.1 mg/mL to 20 mg/mL, from 0.1 mg/mL to 15 mg/mL, from 0.1 mg/mL to 10 mg/mL, from 0.1 mg/mL to 9 mg/mL, from 0.1 mg/mL to 8 mg/mL, from 0.1 mg/mL to 7 mg/mL, from 0.1 mg/mL to 6 mg/mL, from 0.1 mg/mL to 5 mg/mL, from 0.1 mg/mL to 4 mg/mL, from 0.1 mg/mL to 3.9 mg/mL, from 0.1 mg/mL to 3.8 mg/mL, from 0.1 mg/mL to 3.7 mg/mL, from 0.1 mg/mL to 3.6 mg/mL, from 0.1 mg/mL to 3.5 mg/mL, from 0.1 mg/mL to 3.4 mg/mL, from 0.1 mg/mL to 3.3 mg/mL, from 0.1 mg/mL to 3.2 mg/mL, from 0.1 mg/mL to 3.1 mg/mL, from 0.1 mg/mL to 3.0 mg/mL, from 0.1 mg/mL to 2.9 mg/mL, from 0.1 mg/mL to 2.8 mg/mL, from 0.1 mg/mL to 2.7 mg/mL, from 0.1 mg/mL to 2.6 mg/mL, from 0.1 mg/mL to 2.5 mg/mL, from 0.1 mg/mL to 2.4 mg/mL, from 0.1 mg/mL to 2.3 mg/mL, from 0.1 mg/mL to 2.2 mg/mL, from 0.1 mg/mL to 2.1 mg/mL, from 0.1 mg/mL to 2.0 mg/mL, from 0.1 mg/mL to 1.9 mg/mL, from 0.1 mg/mL to 1.8 mg/mL, from 0.1 mg/mL to 1.7 mg/mL, from 0.1 mg/mL to 1.6 mg/mL, from 0.1 mg/mL to 1.5 mg/mL, from 0.1 mg/mL to 1.4 mg/mL, from 0.1 mg/mL to 1.3 mg/mL, from 0.1 mg/mL to 1.2 mg/mL, from 0.1 mg/mL to 1.1 mg/mL, from 0.1 mg/mL to 1.0 mg/mL, from 0.1 mg/mL to 0.9 mg/mL, from 0.1 mg/mL to 0.8 mg/mL, from 0.1 mg/mL to 0.7 mg/mL, from 0.1 mg/mL to 0.6 mg/mL, from 0.1 mg/mL to 0.5 mg/mL, from 0.1 mg/mL to 0.4 mg/mL, from 0.1 mg/mL to 0.3 mg/mL, or from 0.1 mg/mL to 0.2 mg/mL.

In some embodiments, the compound as described herein (e.g., compound (I) or a salt thereof, or compound (II)) is solubilized or diluted to a concentration of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent. In some embodiments, the compound as described herein (e.g., compound (I) or a salt thereof, or compound (II)) is solubilized or diluted to a concentration of at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent. In some embodiments, the compound as described herein (e.g., compound (I) or a salt thereof, or compound (II)) is solubilized or diluted to a concentration of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent.

In some embodiments, the pharmaceutical formulation does not comprise a preservative. In some embodiments, the pharmaceutical formulation comprises a preservative.

A pharmaceutical composition used in the therapeutic methods of the invention is formulated to be compatible with its intended route of administration.

A pharmaceutical composition used in the therapeutic methods of the invention is formulated to be delivered to the brain/CNS through the intended route of administration. Routes of delivery to the CNS/brain include, but are not limited to, intrathecal administration (e.g., via the cisterna magna or via lumbar puncture), intracranial administration, e.g., intracerebroventricular administration, or lateral cerebroventricular administration, endovascular administration, and intraparenchymal administration.

In some embodiments, the pharmaceutical formulation is suitable for an intracerebroventricular or intrathecal (IT) injection. Intrathecal administration: As used herein, the term “intrathecal administration” or “intrathecal injection” refers to an injection into the spinal canal (intrathecal space surrounding the spinal cord). Various techniques can be used, including, without limitation, lateral cerebroventricular injection through a lumen or lumen puncture or puncture hole or the like. In some embodiments, “intrathecal administration” or “intrathecal administration” in accordance with the present invention refers to administration or administration of IT through the lumbar area or region, i.e., administration or administration of lumbar IT. As used herein, the term “lumbar region” or “lumbar area” refers to the area between the third and fourth lumbar vertebrae (lower back) and, more inclusive, the L2-S1 region of the spine vertebral.

In some embodiments, the pharmaceutical formulation is suitable for oral, rectal, intranasal, intradermal, subcutaneous, intrathecal, intracerebroventricular, intraperitoneal, intramuscular, intravitreal, intravenous, intracranial, intrabuccal, or sublingual administration. In some embodiments, the pharmaceutical formulation is suitable for intradermal, subcutaneous, intrathecal, intranasal, intracranial, intracerebroventricular, intraperitoneal, intramuscular, intravitreal, or intravenous injection.

In some embodiments, the pharmaceutical formulation is packaged in a single use vial. In some embodiments, the pharmaceutical formulation is packaged in a multiple use vial.

Pharmaceutical formulations comprising the compound, e.g., antisense oligomer, of the described compositions and for use in any of the described methods can be prepared according to conventional techniques well known in the pharmaceutical industry and described in the published literature. In some embodiments, a pharmaceutical formulation for treating a subject comprises an effective amount of any compound as described herein, or a pharmaceutically acceptable salt, solvate, hydrate or ester thereof. In some embodiments, the pharmaceutical composition described herein further comprises a pharmaceutically acceptable excipient, carrier or diluent.

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

A pharmaceutical composition used in the therapeutic methods of the invention is formulated to be compatible with its intended route of administration.

The terms “pharmaceutical composition” and “pharmaceutical formulation” (or “formulation”) are used interchangeably and denote a mixture or solution comprising a therapeutically effective amount of an active pharmaceutical ingredient together with one or more pharmaceutically acceptable excipients to be administered to a subject, e.g., a human in need thereof.

In some embodiments, the compositions are formulated into any of many possible dosage forms such as, but not limited to, solutions, liquids, tablets, capsules, gel capsules, liquid syrups, soft gels, suppositories, and enemas. In some embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some embodiments, a pharmaceutical formulation or composition of the present invention includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In some embodiments, liposomes also include sterically stabilized liposomes, e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the present invention employs a penetration enhancer to effect the efficient delivery of the compound, e.g., oligomer, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple compounds, e.g., multiple antisense oligomers. In some embodiments, the compound (e.g., antisense oligomer) is administered in combination with another drug or therapeutic agent.

Pharmaceutical compositions suitable for injectable use include sterile aqueous solutions (where water soluble) or dispersions and sterile powders for the extemporaneous preparation of sterile injectable solutions or dispersion. Solutions or suspensions used for parenteral, intranasal, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. For example, depending on the injection site, the vehicle may contain water, synthetic or vegetable oil, and/or organic co-solvents. In certain instances, such as with lyophilized product or a concentrate, the parenteral formulation would be reconstituted or diluted prior to administration. pH can be adjusted with acids or bases, such as hydrochloric acid or sodium hydroxide. Depot formulations, providing controlled or sustained release of an invention composition, may include injectable suspensions of nano/micro particles or nano/micro or non-micronized crystals.

For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, poly(ol) (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Sterile injectable solutions can be prepared by incorporating the composition in the required amount in an appropriate solvent with one or a com-bination of ingredients enumerated above, as required, followed by filtered sterilization. Prevention of the action of micro-organisms can be achieved by various antibacterial and antifungal agents, for example, parabens, chlorobutanol, phenol, ascorbic acid, thimerosal, and the like.

Generally, dispersions are prepared by incorporating the active composition into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin. The parenteral preparation can be enclosed in ampoules, disposable syringes or multiple dose vials made of glass or plastic.

For oral administration, the compositions can be formulated in liquid or solid dosage forms and as instant or controlled/sustained release formulations. Suitable dosage forms for oral ingestion by a subject include powders, tablets, pills, granules, dragees, hard- and soft-shell capsules, liquids, gels, syrups, slurries, suspensions, emulsions and the like. Oral compositions generally include an inert diluent or an edible carrier. They can be enclosed in gelatin capsules or compressed into tablets. For the purpose of oral therapeutic administration, the active agent can be incorporated with excipients and used in the form of tablets, troches, or capsules. Oral compositions can also be prepared using a fluid carrier for use as a mouthwash, wherein the agent in the fluid carrier is applied orally and swished and expectorated or swallowed. Pharmaceutically compatible binding agents, and/or adjuvant materials can be included as part of the composition. The tablets, granules, pills, capsules, troches and the like can contain any of the following ingredients, or compounds of a similar nature: a binder such as micro-crystalline cellulose, gum tragacanth or gelatin; an excipient such as starch or lactose; dissolution retardant; anti-adherents; cationic exchange resin; wetting agents; antioxidants; preservatives; a disintegrating agent such as alginic acid, Primogel, or corn starch; a lubricant such as magnesium stearate or Sterotes; a glidant such as colloidal silicon dioxide; a preservative; a colorant; a sweetening agent such as sugars such as dextrose, sucrose or saccharin; or a flavoring agent such as peppermint, methyl salicylate, or orange flavoring, each of these being synthetic and/or natural.

For administration by inhalation, e.g., intranasal administration, the compositions are delivered in the form of an aerosol spray from pressured container or dispenser which contains a suitable propellant, e.g., a gas such as carbon dioxide, or a nebulizer. Systemic administration can also be by transmucosal or trans-dermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents are formulated into ointments, salves, gels, or creams, emulsion, a solution, a suspension, or a foam, as generally known in the art. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustments; use of complexing agents and other techniques, such as iontophoresis, may be used to regulate skin penetration of the active ingredient.

The compositions may also be formulated in rectal compositions, such as suppositories (e.g., with conventional suppository bases such as cocoa butter and other glycerides) or retention enemas.

Examples of pharmaceutically or physiologically acceptable carriers, diluents or excipients include, but are not limited to, antifoaming agents, antioxidants, binders, carriers or carrier materials, dispersing agents, viscosity modulating agents, diluents, filling agents, lubricants, glidants, plasticizers, solubilizers, stabilizers, suspending agents, surfactants, viscosity enhancing agents, and wetting agents.

The separate components of the compositions of the invention may be preblended or each component may be added separately to the same environment according to a predetermined dosage for the purpose of achieving the desired concentration level of the treatment components and so long as the components eventually come into intimate admixture with each other. Further, the invention may be administered or delivered on a continuous or intermittent basis.

It is especially advantageous to formulate oral or parenteral compositions in dosage unit form for ease of administration and uniformity of dosage. Dosage unit form as used herein refers to physically discrete units suited as unitary dosages for the human subject to be treated; each unit containing a predetermined quantity of active compound calculated to produce the desired therapeutic effect in association with the required pharmaceutical carrier. The specification for the dosage unit forms of the invention is dictated by and directly dependent on the unique characteristics of the compositions and the particular therapeutic effect to be achieved, and the limitations inherent in the art of compounding such an agent for the treatment of subjects.

In some embodiments, provided herein is a method of producing the pharmaceutical formulation as described herein.

### Combination Therapies

In some embodiments, the compounds disclosed in the present disclosure can be used in combination with one or more additional therapeutic agents. In some embodiments, the one or more additional therapeutic agents can comprise a small molecule. For example, the one or more additional therapeutic agents can comprise a small molecule described in WO2016128343A1, WO2017053982A1, WO2016196386A1, WO201428459A1, WO201524876A2, WO2013119916A2, and WO2014209841A2, which are incorporated by reference herein in their entirety. In some embodiments, the one or more additional therapeutic agents comprise an ASO that can be used to correct intron retention. In some embodiments, the one or more other agents are selected from the ASOs listed in Table 8A or Table 8B.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder characterized by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the composition to the fetus directly or indirectly (e.g., via the mother).

Suitable routes for administration of compounds of the present disclosure may vary depending on cell type to which delivery of the compounds is desired. Multiple tissues and organs are affected by Dravet syndrome; Epilepsy, generalized, with febrile seizures plus, type 2; Febrile seizures, familial, 3A; Migraine, familial hemiplegic, 3; Autism; Epileptic encephalopathy, early infantile, 13; Sick sinus syndrome 1; Alzheimer's disease or SUDEP, with the brain being the most significantly affected tissue. The compounds of the present disclosure may be administered to patients parenterally, for example, by intrathecal injection, intracerebroventricular injection, intraperitoneal injection, intramuscular injection, subcutaneous injection, intravitreal injection, or intravenous injection.

### Mode of Action

In some embodiments, the reduced expression or function of Na_{V}1.1 protein is associated with an altered splicing of a nonsense-mediated RNA decay-inducing exon (NMD exon) from a pre-mRNA that contains the NMD exon and that encodes Na_{V}1.1 protein. For example, the reduced expression or function of Na_{V}1.1 protein can be associated with a reduced splicing of a nonsense-mediated RNA decay-inducing exon (NMD exon) from a pre-mRNA that contains the NMD exon and that encodes Na_{V}1.1 protein.

In some embodiments, the compound as described herein promotes exclusion of the NMD exon from a pre-mRNA that contains the NMD exon and that encodes Na_{V}1.1 protein.

In some embodiments, the compound as described herein promotes exclusion of the NMD exon from a pre-mRNA that contains the NMD exon and that encodes Na_{V}1.1 protein by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to an untreated control cell, tissue or subject, or compared to the corresponding activity in the same type of cell, tissue or subject before treatment with the compound as described herein as measured by any standard technique. In some embodiments, the compound as described herein promotes exclusion of the NMD exon a pre-mRNA that contains the NMD exon and that encodes Na_{V}1.1 protein by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an untreated control cell, tissue or subject, or compared to the corresponding activity in the same type of cell, tissue or subject before treatment with the compound as described herein as measured by any standard technique.

In some embodiments, the compound as described herein binds to a targeted portion of a pre-mRNA that contains a nonsense-mediated RNA decay-inducing exon and that encodes Na_{V}1.1 protein.

In some embodiments, the compound as described herein promotes exclusion of a nonsense-mediated mRNA decay-inducing exon (NMD exon) from a pre-mRNA that contains the NMD exon and that encodes Na_{V}1.1 protein.

In some embodiments, the compound as described herein promotes exclusion of an NMD exon from a pre-mRNA that contains the NMD exon and that encodes Na_{V}1.1 protein by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to an untreated control cell, tissue or subject, or compared to the corresponding activity in the same type of cell, tissue or subject before treatment with the compound as described herein as measured by any standard technique. In some embodiments, the compound as described herein promotes exclusion of an NMD exon from a pre-mRNA that contains the NMD exon and that encodes Na_{V}1.1 protein by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an untreated control cell, tissue or subject, or compared to the corresponding activity in the same type of cell, tissue or subject before treatment with the compound as described herein as measured by any standard technique.

In some embodiments, the compound as described herein increases a level of processed mRNA encoding the Na_{V}1.1 protein in a cell having a pre-mRNA that contains an NMD exon and that encodes Na_{V}1.1 protein when the compound as described herein (e.g., compound (I) or a salt thereof, or compound (II)) is introduced into the cell.

In some embodiments, when the compound as described herein (e.g., compound (I) or a salt thereof, or compound (II)) is introduced into the cell, the compound as described herein increases a level of processed mRNA encoding the Na_{V}1.1 protein in a cell having a pre-mRNA that contains an NMD exon and that encodes Na_{V}1.1 protein by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to an untreated control cell, tissue or subject, or compared to the corresponding activity in the same type of cell, tissue or subject before treatment with the compound as described herein as measured by any standard technique. In some embodiments, when the compound as described herein (e.g., compound (I) or a salt thereof, or compound (II)) is introduced into the cell, the compound as described herein increases a level of processed mRNA encoding the Na_{V}1.1 protein in a cell having a pre-mRNA that contains an NMD exon and that encodes Na_{V}1.1 protein by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an untreated control cell, tissue or subject, or compared to the corresponding activity in the same type of cell, tissue or subject before treatment with the compound as described herein as measured by any standard technique.

In some embodiments, the compound as described herein increases a level of the Na_{V}1.1 protein in a cell having a pre-mRNA that contains an NMD exon and that encodes Na_{V}1.1 protein when the compound as described herein (e.g., compound (I) or a salt thereof, or compound (II)) is introduced into the cell.

In some embodiments, when the compound as described herein (e.g., compound (I) or a salt thereof, or compound (II)) is introduced into the cell, the compound as described herein increases a level of the Na_{V}1.1 protein in a cell having a pre-mRNA that contains an NMD exon and that encodes Na_{V}1.1 protein by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to an untreated control cell, tissue or subject, or compared to the corresponding activity in the same type of cell, tissue or subject before treatment with the compound as described herein as measured by any standard technique. In some embodiments, when the compound as described herein (e.g., compound (I) or a salt thereof, or compound (II)) is introduced into the cell, the compound as described herein increases a level of the Na_{V}1.1 protein in a cell having a pre-mRNA that contains an NMD exon and that encodes Na_{V}1.1 protein by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an untreated control cell, tissue or subject, or compared to the corresponding activity in the same type of cell, tissue or subject before treatment with the compound as described herein as measured by any standard technique.

In some embodiments, the targeted portion is within an intron sequence flanking the NMD exon.

In some embodiments, the targeted portion comprises at least one nucleotide of the NMD exon.

In some embodiments, the targeted portion comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotide of the NMD exon. In some embodiments, the targeted portion comprises at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotide of the NMD exon.

In some embodiments, the targeted portion is within the NMD exon.

In some embodiments, the NMD exon comprises a sequence with at least 80%, at least 90%, or 100% sequence identity to a sequence selected from the group consisting of the sequences listed in Table 1 or Table 2. In some embodiments, the NMD exon comprises a sequence selected from the group consisting of the sequences listed in Table 1 or Table 2. In some embodiments, the pre-mRNA comprises a sequence with at least about 80%, 85%, 90%, 95%, 97%, or 100% sequence identity to a sequence selected from the group consisting of the sequences listed in Table 1 or Table 2. In some embodiments, the pre-mRNA is encoded by a genetic sequence with at least about 80%, 85%, 90%, 95%, 97%, or 100% sequence identity to a sequence selected from the group consisting of the sequences listed in Table 1 or Table 2. Splicing and Nonsense-Mediated mRNA Decay

Intervening sequences or introns are removed by a large and highly dynamic RNA-protein complex termed the spliceosome, which orchestrates complex interactions between primary transcripts, small nuclear RNAs (snRNAs) and a large number of proteins. Spliceosomes assemble ad hoc on each intron in an ordered manner, starting with recognition of 5′ splice site (5′ss) by U1 snRNA or 3′ splice site (3′ss) by the U2 pathway, which involves binding of the U2 auxiliary factor (U2AF) to 3′ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude. Although they often have a regulatory function, the exact mechanisms of their activation or repression are poorly understood.

The decision of whether to splice or not to splice can be typically modeled as a stochastic rather than deterministic process, such that even the most defined splicing signals can sometimes splice incorrectly. However, under normal conditions, pre-mRNA splicing proceeds at surprisingly high fidelity. This is attributed in part to the activity of adjacent cis-acting auxiliary exonic and intronic splicing regulatory elements (ESRs or ISRs). Typically, these functional elements are classified as either exonic or intronic splicing enhancers (ESEs or ISEs) or silencers (ESSs or ISSs) based on their ability to stimulate or inhibit splicing, respectively. Although there is now evidence that some auxiliary cis-acting elements may act by influencing the kinetics of spliceosome assembly, such as the arrangement of the complex between U1 snRNP and 5′ss, it seems very likely that many elements function in concert with trans-acting RNA-binding proteins (RBPs). For example, the serine- and arginine-rich family of RBPs (SR proteins) is a conserved family of proteins that have a key role in defining exons. SR proteins promote exon recognition by recruiting components of the pre-spliceosome to adjacent splice sites or by antagonizing the effects of ESSs in the vicinity. The repressive effects of ESSs can be mediated by members of the heterogeneous nuclear ribonucleoprotein (hnRNP) family and can alter recruitment of core splicing factors to adjacent splice sites. In addition to their roles in splicing regulation, silencer elements are suggested to have a role in repression of pseudo-exons, sets of decoy intronic splice sites with the typical spacing of an exon but without a functional open reading frame. ESEs and ESSs, in cooperation with their cognate trans-acting RBPs, represent important components in a set of splicing controls that specify how, where and when mRNAs are assembled from their precursors.

The sequences marking the exon-intron boundaries are degenerate signals of varying strengths that can occur at high frequency within human genes. In multi-exon genes, different pairs of splice sites can be linked together in many different combinations, creating a diverse array of transcripts from a single gene. This is commonly referred to as alternative pre-mRNA splicing. Although most mRNA isoforms produced by alternative splicing can be exported from the nucleus and translated into functional polypeptides, different mRNA isoforms from a single gene can vary greatly in their translation efficiency. Those mRNA isoforms with premature termination codons (PTCs) at least 50 bp upstream of an exon junction complex are likely to be targeted for degradation by the nonsense-mediated mRNA decay (NMD) pathway. Mutations in traditional (BPS/PPT/3′ss/5′ss) and auxiliary splicing motifs can cause aberrant splicing, such as exon skipping or cryptic (or pseudo-) exon inclusion or splice-site activation, and contribute significantly to human morbidity and mortality. Both aberrant and alternative splicing patterns can be influenced by natural DNA variants in exons and introns.

Given that exon-intron boundaries can occur at any of the three positions of a codon, it is clear that only a subset of alternative splicing events can maintain the canonical open reading frame. For example, only exons that are evenly divisible by 3 can be skipped or included in the mRNA without any alteration of reading frame. Splicing events that do not have compatible phases will induce a frame-shift. Unless reversed by downstream events, frame-shifts can certainly lead to one or more PTCs, probably resulting in subsequent degradation by NMD. NMD is a translation-coupled mechanism that eliminates mRNAs containing PTCs. NMD can function as a surveillance pathway that exists in all eukaryotes. NMD can reduce errors in gene expression by eliminating mRNA transcripts that contain premature stop codons. Translation of these aberrant mRNAs could, in some cases, lead to deleterious gain-of-function or dominant-negative activity of the resulting proteins. NMD targets not only transcripts with PTCs but also a broad array of mRNA isoforms expressed from many endogenous genes, suggesting that NMD is a master regulator that drives both fine and coarse adjustments in steady-state RNA levels in the cell.

An NMD-inducing exon (NIE) is an exon or a pseudo-exon that is a region within an intron and can activate the NMD pathway if included in a mature RNA transcript. In the constitutive splicing events, the intron containing an NIE is usually spliced out, but the intron or a portion thereof (e.g., NIE) can be retained during alternative or aberrant splicing events. Mature mRNA transcripts containing such an NIE can be non-productive due to frame shift which induce NMD pathway. Inclusion of a NIE in mature RNA transcripts can downregulate gene expression. mRNA transcripts, such as a pre-mRNA transcript, containing an NIE can be referred as “NIE containing mRNA” or “NMD exon mRNA” in the current disclosure.

Cryptic (or pseudo-splice sites) have the same splicing recognition sequences as genuine splice sites but are not used in the splicing reactions. They outnumber genuine splice sites in the human genome by an order of a magnitude and are normally repressed by thus far poorly understood molecular mechanisms. Cryptic 5′ splice sites have the consensus NNN/GUNNNN or NNN/GCNNNN where N is any nucleotide and/is the exon-intron boundary. Cryptic 3′ splice sites have the consensus NAG/N. Their activation is positively influenced by surrounding nucleotides that make them more similar to the optimal consensus of authentic splice sites, namely MAG/GURAGU and YAG/G, respectively, where M is C or A, R is G or A, and Y is C or U.

Splice sites and their regulatory sequences can be readily identified by a skilled person using suitable algorithms publicly available, listed for example in Kralovicova, J. and Vorechovsky, I. (2007) Global control of aberrant splice site activation by auxiliary splicing sequences: evidence for a gradient in exon and intron definition. Nucleic Acids Res., 35, 6399-6413, (http://www.ncbi.nlm.nih.gov/pmc/articles/PMC2095810/pdf/gkm680.pdf)

The cryptic splice sites or splicing regulatory sequences may compete for RNA-binding proteins such as U2AF with a splice site of the NIE. In one embodiment, an agent may bind to the cryptic splice site or splicing regulatory sequences to prevent the binding of RNA-binding proteins and thereby favoring utilization of the NIE splice sites.

In one embodiment, the cryptic splice site may not comprise 5′ or 3′ splice site of the NIE. The cryptic splice site may be at least 10 nucleotides upstream of the NIE 5′ splice site. The cryptic splice site may be at least 20 nucleotides upstream of the NIE 5′ splice site. The cryptic splice site may be at least 50 nucleotides upstream of the NIE 5′ splice site. The cryptic splice site may be at least 100 nucleotides upstream of the NIE 5′ splice site. The cryptic splice site may be at least 200 nucleotides upstream of the NIE 5′ splice site.

The cryptic splice site may be at least 10 nucleotides downstream of the NIE 3′ splice site. The cryptic splice site may be at least 20 nucleotides downstream of the NIE 3′ splice site. The cryptic splice site may be at least 50 nucleotides downstream of the NIE 3′ splice site. The cryptic splice site may be at least 100 nucleotides downstream of the NIE 3′ splice site. The cryptic splice site may be at least 200 nucleotides downstream of the NIE 3′ splice site.

In some embodiments, the methods of the present disclosure exploit the presence of NIE in the pre-mRNA transcribed from the SCN1A gene. Splicing of the identified SCN1A NIE pre-mRNA species to produce functional mature Scn1a mRNA can be induced using a therapeutic agent such as an ASO that stimulates exon skipping of an NIE. Induction of exon skipping can result in inhibition of an NMD pathway. The resulting mature Scn1a mRNA can be translated normally without activating NMD pathway, thereby increasing the amount of Na_{V}1.1 (also termed “Na_{V}1.1 protein” herein) in the patient's cells and alleviating symptoms of a condition associated with SCN1A deficiency, such as Dravet Syndrome (DS); Epilepsy, generalized, with febrile seizures plus, type 2; Febrile seizures, familial, 3A; Autism; Epileptic encephalopathy, early infantile, 13; Sick sinus syndrome 1; Alzheimer's disease; or SUDEP.

Where reference is made to reducing NIE inclusion in the mature mRNA, the reduction may be complete, e.g., 100%, or may be partial. The reduction may be clinically significant. The reduction/correction may be relative to the level of NIE inclusion in the human subject without treatment, or relative to the amount of NIE inclusion in a population of similar subjects. The reduction/correction may be at least 10% less NIE inclusion relative to the average subject, or the human subject prior to treatment. The reduction may be at least 20% less NIE inclusion relative to an average subject, or the human subject prior to treatment. The reduction may be at least 40% less NIE inclusion relative to an average subject, or the human subject prior to treatment. The reduction may be at least 50% less NIE inclusion relative to an average subject, or the human subject prior to treatment. The reduction may be at least 60% less NIE inclusion relative to an average subject, or the human subject prior to treatment. The reduction may be at least 80% less NIE inclusion relative to an average subject, or the human subject prior to treatment. The reduction may be at least 90% less NIE inclusion relative to an average subject, or the human subject prior to treatment.

Where reference is made to increasing active-Na_{V}1.1 protein levels, the increase may be clinically significant. The increase may be relative to the level of active-Na_{V}1.1 protein in the human subject without treatment, or relative to the amount of active-Na_{V}1.1 protein in a population of similar subjects. The increase may be at least 10% more active-Na_{V}1.1 protein relative to the average subject, or the human subject prior to treatment. The increase may be at least 20% more active-Na_{V}1.1 protein relative to the average subject, or the human subject prior to treatment. The increase may be at least 40% more active-Na_{V}1.1 protein relative to the average subject, or the human subject prior to treatment. The increase may be at least 50% more active-Na_{V}1.1 protein relative to the average subject, or the human subject prior to treatment. The increase may be at least 80% more active-Na_{V}1.1 protein relative to the average subject, or the human subject prior to treatment. The increase may be at least 100% more active-Na_{V}1.1 protein relative to the average subject, or the human subject prior to treatment. The increase may be at least 200% more active-Na_{V}1.1 protein relative to the average subject, or the human subject prior to treatment. The increase may be at least 500% more active-Na_{V}1.1 protein relative to the average subject, or the human subject prior to treatment.

### Exon Inclusion

As used herein, a “NIE containing pre-mRNA” is a pre-mRNA transcript that contains at least one pseudo-exon. Alternative or aberrant splicing can result in inclusion of the at least one pseudo-exon in the mature mRNA transcripts. The terms “mature mRNA,” and “fully-spliced mRNA,” are used interchangeably here-in to describe a fully processed mRNA. Inclusion of the at least one pseudo-exon can be non-productive mRNA and lead to NMD of the mature mRNA. NIE containing mature mRNA may sometimes lead to aberrant protein expression.

One of skill in the art also can determine the sequences of flanking exons in any SCN1A isoform for targeting using the methods of the disclosure, based on an intron sequence provided herein or using the intron number provided in reference to the mRNA sequence at NM_006920, NM_001202435, NM_001165964, or NM_001165963.

In some embodiments, the methods and compositions of the present disclosure are used to modulate, e.g., increase or decrease, the expression of SCN1A by inducing or inhibiting exon skipping of a pseudo-exon of an SCN1A NIE containing pre-mRNA. In some embodiments, the pseudo-exon is a sequence within any of introns 1-25. In some embodiments, the pseudo-exon is a sequence within any of introns 2, 4, 6, 13, 14, 15, 16, 17, 18, 20, 21, 22, 23, 24, and 25. In some embodiments, the pseudo-exon is a sequence within any of introns 15, 18, and 19. In some embodiments, the pseudo-exon can be any SCN1A intron or a portion thereof. In some embodiments, the pseudo-exon is within intron 20. The SCN1A intron numbering used herein corresponds to the mRNA sequence at NM_006920. It is understood that the intron numbering may change in reference to a different SCN1A isoform sequence.

In some embodiments, the included pseudo-exon is the most abundant pseudo-exon in a population of NIE containing pre-mRNAs transcribed from the gene encoding the target protein in a cell. In some embodiments, the included pseudo-exon is the most abundant pseudo-exon in a population of NIE containing pre-mRNAs transcribed from the gene encoding the target protein in a cell, where-in the population of NIE containing pre-mRNAs comprises two or more included pseudo-exons. In some embodiments, an antisense oligomer targeted to the most abundant pseudo-exon in the population of NIE containing pre-mRNAs encoding the target protein induces exon skipping of one or two or more pseudo-exons in the population, including the pseudo-exon to which the antisense oligomer is targeted or binds. In some embodiments, the targeted region is in a pseudo-exon that is the most abundant pseudo-exon in a NIE containing pre-mRNA encoding the Na_{V}1.1 protein.

The degree of exon inclusion can be expressed as percent exon inclusion, e.g., the percentage of transcripts in which a given pseudo-exon is included. In brief, percent exon inclusion can be calculated as the percentage of the amount of RNA transcripts with the exon inclusion, over the sum of the average of the amount of RNA transcripts with exon inclusion plus the average of the amount of RNA transcripts with exon exclusion.

In some embodiments, an included pseudo-exon is an exon that is identified as an included pseudo-exon based on a determination of at least about 5%, at least about 10%, at least about 15%, at least about 20%, at least about 25%, at least about 30%, at least about 35%, at least about 40%, at least about 45%, or at least about 50%, inclusion. In some embodiments, a included pseudo-exon is an exon that is identified as a included pseudo-exon based on a determination of about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 15% to about 100%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 20% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 25% to about 100%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, or about 25% to about 35%, inclusion. ENCODE data (described by, e.g., Tilgner, et al., 2012, “Deep sequencing of subcellular RNA fractions shows splicing to be predominantly co-transcriptional in the human genome but inefficient for lncRNAs,” Genome Research 22 (9): 1616-25, of which entire content is incorporated herein by reference) can be used to aid in identifying exon inclusion.

In some embodiments, contacting cells with an ASO that is complementary to a targeted portion of a SCN1A pre-mRNA transcript results in an increase in the amount of Na_{V}1.1 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of Na_{V}1.1 protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a SCN1A pre-mRNA transcript results in a decrease in the amount of Na_{V}1.1 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of Na_{V}1.1 protein produced by the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a SCN1A pre-mRNA transcript results in an increase in the amount of mRNA encoding SCN1A, including the mature mRNA encoding the target protein. In some embodiments, the amount of mRNA encoding Na_{V}1.1 protein, or the mature mRNA encoding the Na_{V}1.1 protein, is increased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding Na_{V}1.1 protein, or the mature mRNA encoding Na_{V}1.1 protein produced in the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the SCN1A NIE containing pre-mRNA.

In some embodiments, contacting cells with an ASO that is complementary to a targeted portion of a SCN1A pre-mRNA transcript results in a decrease in the amount of mRNA encoding SCN1A, including the mature mRNA encoding the target protein. In some embodiments, the amount of mRNA encoding Na_{V}1.1 protein, or the mature mRNA encoding the Na_{V}1.1 protein, is decreased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding Na_{V}1.1 protein, or the mature mRNA encoding Na_{V}1.1 protein produced in the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the SCN1A NIE containing pre-mRNA.

The NIE can be in any length. In some embodiments, the NIE comprises a full sequence of an intron, in which case, it can be referred to as intron retention. In some embodiments, the NIE can be a portion of the intron. In some embodiments, the NIE can be a 5′ end portion of an intron including a 5′ss sequence. In some embodiments, the NIE can be a 3′ end portion of an intron including a 3′ss sequence. In some embodiments, the NIE can be a portion within an intron without inclusion of a 5′ss sequence. In some embodiments, the NIE can be a portion within an intron without inclusion of a 3′ss sequence. In some embodiments, the NIE can be a portion within an intron without inclusion of either a 5′ss or a 3′ss sequence. In some embodiments, the NIE can be from 5 nucleotides to 10 nucleotides in length, from 10 nucleotides to 15 nucleotides in length, from 15 nucleotides to 20 nucleotides in length, from 20 nucleotides to 25 nucleotides in length, from 25 nucleotides to 30 nucleotides in length, from 30 nucleotides to 35 nucleotides in length, from 35 nucleotides to 40 nucleotides in length, from 40 nucleotides to 45 nucleotides in length, from 45 nucleotides to 50 nucleotides in length, from 50 nucleotides to 55 nucleotides in length, from 55 nucleotides to 60 nucleotides in length, from 60 nucleotides to 65 nucleotides in length, from 65 nucleotides to 70 nucleotides in length, from 70 nucleotides to 75 nucleotides in length, from 75 nucleotides to 80 nucleotides in length, from 80 nucleotides to 85 nucleotides in length, from 85 nucleotides to 90 nucleotides in length, from 90 nucleotides to 95 nucleotides in length, or from 95 nucleotides to 100 nucleotides in length. In some embodiments, the NIE can be at least 10 nucleotides, at least 20 nucleotides, at least 30 nucleotides, at least 40 nucleotides, at least 50 nucleotides, at least 60 nucleoids, at least 70 nucleotides, at least 80 nucleotides in length, at least 90 nucleotides, or at least 100 nucleotides in length. In some embodiments, the NIE can be from 100 to 200 nucleotides in length, from 200 to 300 nucleotides in length, from 300 to 400 nucleotides in length, from 400 to 500 nucleotides in length, from 500 to 600 nucleotides in length, from 600 to 700 nucleotides in length, from 700 to 800 nucleotides in length, from 800 to 900 nucleotides in length, from 900 to 1,000 nucleotides in length. In some embodiments, the NIE may be longer than 1,000 nucleotides in length.

Inclusion of a pseudo-exon can lead to a frameshift and the introduction of a premature termination codon (PIC) in the mature mRNA transcript rendering the transcript a target of NMD. Mature mRNA transcript containing NIE can be non-productive mRNA transcript which does not lead to protein expression. The PIC can be present in any position downstream of an NIE. In some embodiments, the PIC can be present in any exon downstream of an NIE. In some embodiments, the PIC can be present within the NIE. For example, inclusion of exon 20x in an mRNA transcript encoded by the SCN1A gene can induce a PIC in the mRNA transcript, e.g., a PIC in exon 21 of the mRNA transcript.

ASOs that when hybridized to a region of a pre-mRNA result in exon skipping (or enhanced splicing of the intron containing a NIE) and increased protein production may be tested in vivo using animal models, for example transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intrathecal injection, intracerebroventricular injection, intraperitoneal injection, intramuscular injection, subcutaneous injection, intravitreal injection, or intravenous injection. Following administration, the cells, tissues, and/or organs of the model animals may be assessed to determine the effect of the treatment by for example evaluating splicing (efficiency, rate, extent) and protein production by methods known in the art and described herein. The animal models may also be any phenotypic or behavioral indication of the disease or disease severity.

In some embodiments, the compound (e.g., ASO) is administered with one or more agents capable of promoting penetration of the human subject compound across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In some embodiments, the compounds (e.g., ASOs) are linked or conjugated with agents that provide desirable pharmaceutical or pharmacodynamic properties. In some embodiments, the compound is coupled to a substance, known in the art to promote penetration or transport across the blood-brain barrier, e.g., an antibody to the transferrin receptor. In some embodiments, the compound is linked with a viral vector, e.g., to render the antisense compound more effective or increase transport across the blood-brain barrier. In some embodiments, osmotic blood brain barrier disruption is assisted by infusion of sugars, e.g., meso erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. Methods and materials for enhancing blood brain barrier penetration are described, e.g., in U.S. Pat. No. 9,193,969, “Compositions and methods for selective delivery of oligonucleotide molecules to specific neuron types,” U.S. Pat. No. 4,866,042, “Method for the delivery of genetic material across the blood brain barrier,” U.S. Pat. No. 6,294,520, “Material for passage through the blood-brain barrier,” and U.S. Pat. No. 6,936,589, “Parenteral delivery systems,” each incorporated herein by reference.

In some embodiments, a compound of the disclosure is coupled to a dopamine reuptake inhibitor (DRI), a selective serotonin reuptake inhibitor (SSRI), a noradrenaline reuptake inhibitor (NRI), a norepinephrine-dopamine reuptake inhibitor (NDRI), and a serotonin-norepinephrine-dopamine reuptake inhibitor (SNDRI), using methods described in, e.g., U.S. Pat. No. 9,193,969, incorporated herein by reference.

In some embodiments, subjects treated using the methods and compositions are evaluated for improvement in condition using any methods known and described in the art.

In some embodiments, the SCN1A NIE containing pre-mRNA transcript is encoded by a genetic sequence with at least about 80%, 85%, 90%, 95%, 96%, 97%, 98%, 99% or 100% sequence identity to any one of the sequences listed in Table 1 or Table 2. In some embodiments, the SCN1A NIE pre-mRNA transcript comprises a sequence with at least about 80%, 85%, 90%, 95%, 96%, 97%, 98%, 99% or 100% sequence identity to any one of the sequences listed in Table 1 or Table 2.

In some embodiments, the SCN1A NIE containing pre-mRNA transcript comprises a sequence with at least about 80%, 85%, 90%, 95%, 97%, or 100% sequence identity to any one of the sequences listed in Table 1 or Table 2. In some embodiments, SCN1A NIE containing pre-mRNA transcript is encoded by a sequence with at least about 80%, 85%, 90%, 95%, 97%, or 100% sequence identity to any one of the sequences listed in Table 1 or Table 2. In some embodiments, the targeted portion of the pre-mRNA containing an NMD exon and encoding Na_{V}1.1 comprises a sequence with at least 80%, 85%, 90%, 95%, 97%, or 100% sequence identity to a region comprising at least 8 contiguous nucleic acids of any one of the sequences listed in Table 1 or Table 2.

In some embodiments, the pre-mRNA transcript comprises a sequence with at least about 80%, 85%, 90%, 95%, 96%, 97%, 98%, 99% or 100% sequence identity to a pre-mRNA transcript of SCN1A pre-mRNA transcripts or a complement thereof described herein. In some embodiments, the targeted portion of the pre-mRNA selected from the group consisting of SCN1A pre-mRNAs comprises a sequence with at least 80%, 85%, 90%, 95%, 97%, or 100% sequence identity to a region comprising at least 8 contiguous nucleic acids of a sequence of the pre-mRNA transcripts of Table 1 or Table 2 or complements thereof. In some embodiments, the targeted portion of the pre-mRNA of SCN1A pre-mRNA comprises a sequence that is complementary to at least 8, 9, 10, 11, 12, 13, 14, 15, 16, 17, 18, 19 or 20 contiguous nucleic acids of a sequence of Table 1 or Table 2 or a complement thereof.

In some embodiments, the pre-mRNA transcript comprises a sequence with at least about 80%, 85%, 90%, 95%, 96%, 97%, 98%, 99% or 100% sequence identity to a pre-mRNA transcript of SCN1A pre-mRNA transcripts or a complement thereof described herein. In some embodiments, the targeted portion of the pre-mRNA selected from the group consisting of SCN1A pre-mRNAs comprises a sequence with at least 80%, 85%, 90%, 95%, 97%, or 100% sequence identity to a region comprising at least 8 contiguous nucleic acids of a sequence of the pre-mRNA transcripts of Table 1 or Table 2 or complements thereof. In some embodiments, the targeted portion of the pre-mRNA of SCN1A pre-mRNA comprises a sequence that is complementary to at least 8, 9, 10, 11, 12, 13, 14, 15, 16, 17, 18, 19 or 20 contiguous nucleic acids of a sequence of Table 1 or Table 2 or a complement thereof.

In some embodiments, the targeted portion of the SCN1A pre-mRNA comprises a sequence with at least 80%, 85%, 90%, 95%, 97%, or 100% sequence identity to a region comprising at least 8 contiguous nucleic acids of a sequence of sequence of Table 3 or complements thereof. In some embodiments, the targeted portion of the SCN1A pre-mRNA comprises a sequence with at least 80%, 85%, 90%, 95%, 97%, or 100% sequence identity to a region comprising at least 8 contiguous nucleic acids of a sequence selected from the group consisting of sequences listed in Table 2 or Table 3 or complements thereof.

In some embodiments, the ASO has a sequence complementary to the targeted portion of the pre-mRNA containing an NMD exon according to any one of the sequences listed in Table 1 or Table 2. In some embodiments, the ASO targets a sequence upstream from the 5′ end of an NIE. For example, ASOs targeting a sequence upstream from the 5′ end of an NIE comprises a sequence that is at least about 80%, 85%, 90%, 95%, 97%, or 100% complementary to at least 8 contiguous nucleic acids of any one of the sequences listed in Table 1 or Table 2. For example, ASOs targeting a sequence upstream from the 5′ end of an NIE can comprise a sequence with at least 80%, 85%, 90%, 95%, 97%, or 100% sequence identity to any one of the sequences listed in Table 1 or Table 2.

In some embodiments, the ASOs target a sequence containing an exon-intron boundary (or junction). For example, ASOs targeting a sequence containing an exon-intron boundary can comprise a sequence that is at least about 80%, 85%, 90%, 95%, 97%, or 100% complementary to at least 8 contiguous nucleic acids of any one of the sequences listed in Table 1 or Table 2. In some embodiments, the ASOs target a sequence downstream from 3′ end of an NIE. For example, ASOs targeting a sequence downstream from 3′ end of an NIE can comprise a sequence with at least 80%, 85%, 90%, 95%, 97%, or 100% sequence identity to any one of the sequences listed in Table 1 or Table 2. For example, ASOs targeting a sequence downstream from 3′ end of an NIE can comprise a sequence with at least 80%, 85%, 90%, 95%, 97%, or 100% sequence identity to any one of the sequences listed in Table 1 or Table 2. In some embodiments, ASOs target a sequence within an NIE.

Methods of Identifying Additional ASOs that Induce Exon Skipping

Also within the scope of the present disclosure are methods for identifying or determining ASOs that induce exon skipping of a SCN1A NIE containing pre-mRNA. For example, a method can comprise identifying or determining ASOs that induce pseudo-exon skipping of a SCN1A NIE containing pre-mRNA. ASOs that specifically hybridize to different nucleotides within the target region of the pre-mRNA may be screened to identify or determine ASOs that improve the rate and/or extent of splicing of the target intron. In some embodiments, the ASO may block or interfere with the binding site(s) of a splicing repressor(s)/silencer. Any method known in the art may be used to identify (determine) an ASO that when hybridized to the target region of the exon results in the desired effect (e.g., pseudo-exon skipping, protein or functional RNA production). These methods also can be used for identifying ASOs that induce exon skipping of the included exon by binding to a targeted region in an intron flanking the included exon, or in a non-included exon. An example of a method that may be used is provided below.

A round of screening, referred to as an ASO “walk” may be performed using ASOs that have been designed to hybridize to a target region of a pre-mRNA. For example, the ASOs used in the ASO walk can be tiled every 5 nucleotides from approximately 100 nucleotides upstream of 3′ splice site of the included exon (e.g., a portion of sequence of the exon located upstream of the target/included exon) to approximately 100 nucleotides downstream of 3′ splice site of the target/included exon and/or from approximately 100 nucleotides upstream of 5′ splice site of the included exon to approximately 100 nucleotides downstream of 5′ splice site of the target/included exon (e.g., a portion of sequence of the exon located downstream of the target/included exon). For example, a first ASO of 15 nucleotides in length may be designed to specifically hybridize to nucleotides +6 to +20 relative to 3′ splice site of the target/included exon. A second ASO may be designed to specifically hybridize to nucleotides +11 to +25 relative to 3′ splice site of the target/included exon. ASOs are designed as such spanning the target region of the pre-mRNA. In some embodiments, the ASOs can be tiled more closely, e.g., every 1, 2, 3, or 4 nucleotides. Further, the ASOs can be tiled from 100 nucleotides downstream of 5′ splice site, to 100 nucleotides upstream of 3′ splice site. In some embodiments, the ASOs can be tiled from about 1,160 nucleotides upstream of 3′ splice site, to about 500 nucleotides downstream of 5′ splice site. In some embodiments, the ASOs can be tiled from about 500 nucleotides upstream of 3′ splice site, to about 1,920 nucleotides downstream of the 3′ splice site.

One or more ASOs, or a control ASO (an ASO with a scrambled sequence, sequence that is not expected to hybridize to the target region) are delivered, for example by transfection, into a disease-relevant cell line that expresses the target pre-mRNA (e.g., a NIE containing pre-mRNA described herein). The exon skipping effects of each of the ASOs may be assessed by any method known in the art, for example by reverse transcriptase (RT)-PCR using primers that span the splice junction. A reduction or absence of a longer RT-PCR product produced using the primers spanning the region containing the included exon (e.g., including the flanking exons of the NIE) in ASO-treated cells as compared to in control ASO-treated cells indicates that splicing of the target NIE has been enhanced. In some embodiments, the exon skipping efficiency (or the splicing efficiency to splice the intron containing the NIE), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in exon skipping (or enhanced splicing of NIE).

Regions defined by ASOs that promote splicing of the target intron are explored in greater detail by means of an ASO “micro-walk”, involving ASOs spaced in 1-nt steps, as well as longer ASOs, typically 18-25 nt.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target pre-mRNA. The splicing-inducing effects of each of the ASOs may be assessed by any method known in the art, for example by reverse transcriptase (RT)-PCR using primers that span the NIE, as described herein. A reduction or absence of a longer RT-PCR product produced using the primers spanning the NIE in ASO-treated cells as compared to in control ASO-treated cells indicates that exon skipping (or splicing of the target intron containing an NIE) has been enhanced. In some embodiments, the exon skipping efficiency (or the splicing efficiency to splice the intron containing the NIE), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

### Kits and Compositions

In some aspects, provided herein is a kit comprising: a concentrate comprising a compound disclosed herein and a diluent, wherein the concentrate is miscible with the diluent; and instructions for diluting or solubilizing the compound in the diluent. In some aspects, provided herein is a kit comprising: a concentrate comprising an antisense oligomer (ASO), wherein the ASO comprises a sequence with at least 80% sequence identity to any one of SEQ ID NOs: 21-67, 210-256 or 304-1099; and a diluent, wherein the concentrate is miscible with the diluent; and instructions for diluting or solubilizing the ASO in the diluent. In some embodiments, the ASO comprises a sequence with at least 80% sequence identity to any one of the sequences listed in listed in Tables 4A, 4B, 5A, 5B, 6A, 6B, 7, 8A, and 8B.

In some embodiments, the ASO comprises a sequence with at least 60%, 65%, 66%, 67%, 68%, 69%, 70%, 71%, 72%, 73%, 74%, 75%, 76%, 77%, 78%, 79%, 80%, 81%, 82%, 83%, 884%, 85%, 86%, 87%, 88%, 89%, 90%, 91%, 92%, 93%, 94%, 95%, 96%, 97%, 98%, 99%, 99.5%, 99.8%, 99.9%, or 100% sequence identity to any one of SEQ ID NOs: 21-67, 210-256 or 304-1099. In some embodiments, the ASO consists of a sequence with at least 60%, 65%, 66%, 67%, 68%, 69%, 70%, 71%, 72%, 73%, 74%, 75%, 76%, 77%, 78%, 79%, 80%, 81%, 82%, 83%, 884%, 85%, 86%, 87%, 88%, 89%, 90%, 91%, 92%, 93%, 94%, 95%, 96%, 97%, 98%, 99%, 99.5%, 99.8%, 99.9%, or 100% sequence identity to any one of SEQ ID NOs: 21-67, 210-256 or 304-1099. In some embodiments, the ASO comprises a sequence with at least 60%, 65%, 66%, 67%, 68%, 69%, 70%, 71%, 72%, 73%, 74%, 75%, 76%, 77%, 78%, 79%, 80%, 81%, 82%, 83%, 884%, 85%, 86%, 87%, 88%, 89%, 90%, 91%, 92%, 93%, 94%, 95%, 96%, 97%, 98%, 99%, 99.5%, 99.8%, 99.9%, or 100% sequence identity to any one of the sequences listed in listed in Tables 4A, 4B, 5A, 5B, 6A, 6B, 7, 8A, and 8B. In some embodiments, the ASO consists of a sequence with at least 60%, 65%, 66%, 67%, 68%, 69%, 70%, 71%, 72%, 73%, 74%, 75%, 76%, 77%, 78%, 79%, 80%, 81%, 82%, 83%, 884%, 85%, 86%, 87%, 88%, 89%, 90%, 91%, 92%, 93%, 94%, 95%, 96%, 97%, 98%, 99%, 99.5%, 99.8%, 99.9%, or 100% sequence identity to any one of the sequences listed in listed in Tables 4A, 4B, 5A, 5B, 6A, 6B, 7, 8A, and 8B.

In some embodiments, the diluent is an artificial cerebral spinal fluid (aCSF) solution. In some embodiments, the solution comprises a cerebral spinal fluid (CSF) sample from the human subject. In some embodiments, the diluent comprises an isotonic solution. In some embodiments, the diluent comprises a phosphate-buffered (pH 6.6-7.6) solution.

In some embodiments, the compound as described herein (e.g., compound (I) or a salt thereof, or compound (II)) is solubilized or diluted in a phosphate-buffered (pH 6.6-7.6) solution. In some embodiments, the compound as described herein (e.g., compound (I) or a salt thereof, or compound (II)) is solubilized or diluted in a phosphate-buffered (pH 6.0-8.0) solution. In some embodiments, the compound as described herein (e.g., compound (I) or a salt thereof, or compound (II)) is solubilized or diluted in a phosphate-buffered (pH 5.0-8.0) solution. In some embodiments, the compound as described herein (e.g., compound (I) or a salt thereof, or compound (II)) is solubilized or diluted in a phosphate-buffered solution with pH 4.5-8.5, pH 4.6-8.5, pH 4.7-8.5, pH 4.8-8.5, pH 4.9-8.5, pH5.0-8.5, pH 5.1-8.5, pH5.2-8.5, pH 5.3-8.5, pH5.4-8.5, pH 5.5-8.5, pH5.6-8.5, pH 5.7-8.5, pH 5.8-8.5, H 5.9-8.5, pH 6.0-8.5, pH 6.1-8.5, pH 6.2-8.5, pH 6.3-8.5, pH 6.4-8.5, pH 6.5-8.5, pH 6.6-8.5, pH 6.7-8.5, pH 6.8-8.5, pH 6.9-8.5, pH 7.0-8.5, pH 7.1-8.5, pH 7.2-8.5, pH 7.3-8.5, pH 7.4-8.5, pH 7.5-8.5, pH 7.6-8.5, pH 7.7-8.5, pH 7.8-8.5, pH 7.9-8.5, pH 8.0-8.5, pH 8.1-8.5, pH 8.2-8.5, pH 8.3-8.5, or pH 8.4-8.5. In some embodiments, the compound as described herein (e.g., compound (I) or a salt thereof, or compound (II)) is solubilized or diluted in a phosphate-buffered solution with pH 4.5-8.3, pH 4.5-8.2, pH 4.5-8.1, pH 4.5-8.0, pH 4.5-7.9, pH 4.5-7.8, pH 4.5-7.7, pH 4.5-7.6, pH 4.5-7.5, pH 4.5-7.4, pH 4.5-7.3, pH 4.5-7.2, pH 4.5-7.1, pH 4.5-7.0, pH 4.5-6.9, pH 4.5-6.8, pH 4.5-6.7, pH 4.5-6.6, pH 4.5-6.5, pH 4.5-6.4, pH 4.5-6.3, pH 4.5-6.2, pH 4.5-6.1, pH 4.5-6.0, pH 4.5-5.9, pH 4.5-5.8, pH 4.5-5.7, pH 4.5-5.6, pH 4.5-5.5, pH 4.5-5.4, pH 4.5-5.3, pH 4.5-5.2, pH 4.5-5.1, pH 4.5-5.0, pH 4.5-4.9, pH 4.5-4.8, pH 4.5-4.7, or pH 4.5-4.6. In some embodiments, the compound as described herein (e.g., compound (I) or a salt thereof, or compound (II)) is solubilized or diluted in a phosphate-buffered solution with pH 6.0-7.6, pH 6.1-7.6, pH 6.2-7.6, pH 6.3-7.6, pH 6.4-7.6, pH 6.5-7.6, pH 6.6-7.6, pH 6.7-7.6, pH 6.8-7.6, pH 6.9-7.6, pH 7.0-7.6, pH 7.1-7.6, pH 7.2-7.6, pH 7.3-7.6, pH 7.4-7.6, or pH 7.5-7.6. In some embodiments, the compound as described herein (e.g., compound (I) or a salt thereof, or compound (II)) is solubilized or diluted in a phosphate-buffered solution with pH 6.6-8.0, pH 6.6-7.9, pH 6.6-7.8, pH 6.6-7.7, pH 6.6-7.6, pH 6.6-7.5, pH 6.6-7.4, pH 6.6-7.3, pH 6.6-7.2, pH 6.6-7.1, pH 6.6-7.0, pH 6.6-6.9, pH 6.6-6.8, or pH 6.6-6.7. In some embodiments, the compound as described herein (e.g., compound (I) or a salt thereof, or compound (II)) is solubilized or diluted in a phosphate-buffered solution with pH 6.0-8.0, pH 6.1-8.0, pH 6.2-8.0, pH 6.3-8.0, pH 6.4-8.0, pH 6.5-8.0, pH 6.6-8.0, pH 6.7-8.0, pH 6.8-8.0, pH 6.9-8.0, pH 7.0-8.0, pH 7.1-8.0, pH 7.2-8.0, pH 7.3-8.0, pH 7.4-8.0, pH 7.5-8.0, pH 7.6-8.0, pH 7.7-8.0, pH 7.8-8.0, or pH 7.9-8.0. In some embodiments, the compound as described herein (e.g., compound (I) or a salt thereof, or compound (II)) is solubilized or diluted in a phosphate-buffered solution with pH 6.0-7.9, pH 6.0-7.8, pH 6.0-7.7, pH 6.0-7.6, pH 6.0-7.5, pH 6.0-7.4, pH 6.0-7.3, pH 6.0-7.2, pH 6.0-7.1, pH 6.0-7.0, pH 6.0-6.9, pH 6.0-6.8, pH 6.0-6.7, pH 6.0-6.6, pH 6.0-6.5, pH 6.0-6.4, pH 6.0-6.3, pH 6.0-6.2, or pH 6.0-6.1. In some embodiments, the compound as described herein (e.g., compound (I) or a salt thereof, or compound (II)) is solubilized or diluted in a phosphate-buffered solution with pH 5.7-8.5, 5.8-8.4, 5.9-8.3, 6.0-8.2, 6.1-8.1, 6.2-8.0, 6.3-7.9, 6.4-7.8, 6.5-7.7, or 6.6-7.6. In some embodiments, the compound as described herein (e.g., compound (I) or a salt thereof, or compound (II)) is solubilized or diluted in a phosphate-buffered solution with pH about 5.0, 5.1, 5.2, 5.3, 5.4, 5.5, 5.6, 5.7, 5.8, 5.9, 6.0. 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0. In some embodiments, the compound as described herein (e.g., compound (I) or a salt thereof, or compound (II)) is solubilized or diluted in a phosphate-buffered solution with pH 5.0, 5.1, 5.2, 5.3, 5.4, 5.5, 5.6, 5.7, 5.8, 5.9, 6.0. 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

In some embodiments, the diluent comprises 25-250 mM NaCl.

In some embodiments, the diluent comprises 25-250, 30-250, 35-250, 40-250, 45-250, 50-250, 55-250, 60-250, 65-250, 70-250, 75-250, 80-250, 85-250, 90-250, 95-250, 100-250, 105-250, 110-250, 115-250, 120-250, 125-250, 130-250, 135-250, 140-250, 145-250, 150-250, 155-250, 160-250, 165-250, 170-250, 175-250, 180-250, 185-250, 190-250, 195-250, 200-250, 205-250, 210-250, 215-250, 220-250, 225-250, 230-250, 235-250, 240-250, or 245-250 mM NaCl. In some embodiments, the diluent comprises 25-245, 25-240, 25-235, 25-230, 25-225, 25-220, 25-215, 25-210, 25-205, 25-200, 25-195, 25-190, 25-185, 25-180, 25-175, 25-170, 25-165, 25-160, 25-155, 25-150, 25-145, 25-140, 25-135, 25-130, 25-125, 25-120, 25-115, 25-110, 25-105, 25-110, 25-105, 25-100, 25-95, 25-90, 25-85, 25-80, 25-75, 25-70, 25-65, 25-60, 25-55, 25-50, 25-45, 25-40, 25-35, or 25-30 mM NaCl. In some embodiments, the diluent comprises 30-245, 35-240, 40-235, 45-230, 50-225, 55-220, 60-215, 65-210, 70-205, 75-200, 80-195, 85-190, 90-185, 95-180, 100-175, 105-170, 110-165, 115-160, 120-155, 125-150, 130-145 or 135-140 mM NaCl. In some embodiments, the diluent comprises 100-140, 101-140, 102-140, 103-140, 104-140, 105-140, 106-140, 107-140, 108-140, 109-140, 110-140, 111-140, 112-140, 113-140, 114-140, 115-140, 116-140, 117-140, 118-140, 119-140, 120-140, 121-140, 122-140, 123-140, 124-140, 125-140, 126-140, 127-140, 128-140, 129-140, 130-140, 131-140, 132-140, 133-140, 134-140, 135-140, 136-140, 137-140, 138-140, or 139-140 mM NaCl. In some embodiments, the diluent comprises 100-139, 100-138, 100-137, 100-136, 100-135, 100-134, 100-133, 100-132, 100-131, 100-130, 100-129, 100-128, 100-127, 100-126, 100-125, 100-124, 100-123, 100-122, 100-121, 100-120, 100-119, 100-118, 100-117, 100-116, 100-115, 100-114, 100-113, 100-112, 100-111, 100-110, 100-109, 100-108, 100-107, 100-106, 100-105, 100-104, 100-103, 100-102, or 100-101 mM NaCl. In some embodiments, the diluent comprises at least 90, 91, 92, 93, 94, 95, 96, 97, 98, 99, 100, 101, 102, 103, 104, 105, 106, 107, 108, 109, 110, 111, 112, 113, 114, 115, 116, 118, 119, 120, 121, 122, 123, 124, 125, 126, 127, 128, 129, 130, 131, 132, 133, 134, 135, 136, 137, 138, 139, or 140 mM NaCl. In some embodiments, the diluent comprises at most 90, 91, 92, 93, 94, 95, 96, 97, 98, 99, 100, 101, 102, 103, 104, 105, 106, 107, 108, 109, 110, 111, 112, 113, 114, 115, 116, 118, 119, 120, 121, 122, 123, 124, 125, 126, 127, 128, 129, 130, 131, 132, 133, 134, 135, 136, 137, 138, 139, or 140 mM NaCl. In some embodiments, the diluent comprises 90, 91, 92, 93, 94, 95, 96, 97, 98, 99, 100, 101, 102, 103, 104, 105, 106, 107, 108, 109, 110, 111, 112, 113, 114, 115, 116, 118, 119, 120, 121, 122, 123, 124, 125, 126, 127, 128, 129, 130, 131, 132, 133, 134, 135, 136, 137, 138, 139, or 140 mM NaCl.

In some embodiments, the diluent comprises 0.1-20 mM KCl.

In some embodiments, the diluent comprises 0.1-40, 0.1-39, 0.1-38, 0.1-37, 0.1-36, 0.1-35, 0.1-34, 0.1-33, 0.1-32, 0.1-31, 0.1-30, 0.1-29, 0.1-28, 0.1-27, 0.1-26, 0.1-25, 0.1-24, 0.1-23, 0.1-22, 0.1-21, 0.1-20, 0.1-19, 0.1-18, 0.1-17, 0.1-16, 0.1-15, 0.1-14, 0.1-13, 0.1-12, 0.1-10, 0.1-9, 0.1-8, 0.1-7, 0.1-6, 0.1-5, 0.1-4, 0.1-3, 0.1-2, or 0.1-1 mM KCl. In some embodiments, the diluent comprises 0.2-40, 0.3-40, 0.4-40, 0.5-40, 0.6-40, 0.7-40, 0.8-40, 0.9-40, 1-40, 2-40, 3-40, 4-40, 5-40, 6-40, 7-40, 8-40, 9-40, 10-40, 11-40, 12-40, 13-40, 14-40, 15-40, 16-40, 17-40, 18-40, 19-40, 20-40, 21-40, 22-40, 23-40, 24-40, 25-40, 26-40, 27-40, 28-40, 29-40, 30-40, 31-40, 32-40, 33-40, 34-40, 35-40, 36-40, 37-40, 38-40, or 39-40 mM KCl. In some embodiments, the diluent comprises 0.1-3.5, 0.2-3.5, 0.3-3.5, 0.4-3.5, 0.5-3.5, 0.6-3.5, 0.7-3.5, 0.8-3.5, 0.9-3.5, 1.0-3.5, 1.1-3.5, 1.2-3.5, 1.3-3.5, 1.4-3.5, 1.5-3.5, 1.6-3.5, 1.7-3.5, 1.8-3.5, 1.9-3.5, 2.0-3.5, 2.1-3.5, 2.2-3.5, 2.3-3.5, 2.4-3.5, 2.5-3.5, 2.6-3.5, 2.7-3.5, 2.8-3.5, 2.9-3.5, 3.0-3.5, 3.1-3.5, 3.2-3.5, 3.3-3.5, or 3.4-3.5 mM KCl. In some embodiments, the diluent comprises 0.1-3.4, 0.1-3.3, 0.1-3.2, 0.1-3.1, 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM KCl. In some embodiments, the diluent comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 mM KCl. In some embodiments, the diluent comprises at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 mM KCl. In some embodiments, the diluent comprises 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, or 3.5 mM KCl.

In some embodiments, the diluent comprises 0-50 mM Na₂HPO₄.

In some embodiments, the diluent comprises 0-50, 0-45, 0-40, 0-35, 0-30, 0-25, 0-20, 0-19, 0-18, 0-17, 0-16, 0-15, 0-14, 0-13, 0-12, 0-11, 0-10, 0-9, 0-8, 0-7, 0-6, 0-5, 0-4, 0-3, 0-2, 0-1, 0-0.9, 0-0.8, 0-0.7, 0-0.6, 0-0.5, 0-0.4, 0-0.3, or 0-0.2 mM Na₂HPO₄. In some embodiments, the diluent comprises 0.1-50, 0.1-45, 0.1-40, 0.1-35, 0.1-30, 0.1-25, 0.1-20, 0.1-19, 0.1-18, 0.1-17, 0.1-16, 0.1-15, 0.1-14, 0.1-13, 0.1-12, 0.1-11, 0.1-10, 0.1-9, 0.1-8, 0.1-7, 0.1-6, 0.1-5, 0.1-4, 0.1-3, 0.1-2, 0.1-1, 0.1-0.9, 0.1-0.8, 0.1-0.7, 0.1-0.6, 0.1-0.5, 0.1-0.4, 0.1-0.3, or 0.1-0.2 mM Na₂HPO₄. In some embodiments, the diluent comprises 0-50, 0.1-50, 0.2-50, 0.3-50, 0.4-50, 0.5-50, 0.6-50, 0.7-50, 0.8-50, 0.9-50, 1-50, 2-50, 3-50, 4-50, 5-50, 6-50, 7-50, 8-50, 9-50, 1-50, 11-50, 12-50, 13-50, 14-50, 15-50, 16-50, 17-50, 18-50, 19-50, 20-50, 25-50, 30-50, 35-50, 40-50, or 45-50 mM Na₂HPO₄. In some embodiments, the diluent comprises 0-20, 0.1-20, 0.2-20, 0.3-20, 0.4-20, 0.5-20, 0.6-20, 0.7-20, 0.8-20, 0.9-20, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, or 19-20 mM Na₂HPO₄. In some embodiments, the diluent comprises at least 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mM Na₂HPO₄. In some embodiments, the diluent comprises at most 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mM Na₂HPO₄. In some embodiments, the diluent comprises 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mM Na₂HPO₄.

In some embodiments, the diluent comprises 0-50 mM NaH₂PO₄.

In some embodiments, the diluent comprises 0-50, 0-45, 0-40, 0-35, 0-30, 0-25, 0-20, 0-19, 0-18, 0-17, 0-16, 0-15, 0-14, 0-13, 0-12, 0-11, 0-10, 0-9, 0-8, 0-7, 0-6, 0-5, 0-4, 0-3, 0-2, 0-1, 0-0.9, 0-0.8, 0-0.7, 0-0.6, 0-0.5, 0-0.4, 0-0.3, or 0-0.2 mM NaH₂PO₄. In some embodiments, the diluent comprises 0.1-50, 0.1-45, 0.1-40, 0.1-35, 0.1-30, 0.1-25, 0.1-20, 0.1-19, 0.1-18, 0.1-17, 0.1-16, 0.1-15, 0.1-14, 0.1-13, 0.1-12, 0.1-11, 0.1-10, 0.1-9, 0.1-8, 0.1-7, 0.1-6, 0.1-5, 0.1-4, 0.1-3, 0.1-2, 0.1-1, 0.1-0.9, 0.1-0.8, 0.1-0.7, 0.1-0.6, 0.1-0.5, 0.1-0.4, 0.1-0.3, or 0.1-0.2 mM NaH₂PO₄. In some embodiments, the diluent comprises 0-50, 0.1-50, 0.2-50, 0.3-50, 0.4-50, 0.5-50, 0.6-50, 0.7-50, 0.8-50, 0.9-50, 1-50, 2-50, 3-50, 4-50, 5-50, 6-50, 7-50, 8-50, 9-50, 1-50, 11-50, 12-50, 13-50, 14-50, 15-50, 16-50, 17-50, 18-50, 19-50, 20-50, 25-50, 30-50, 35-50, 40-50, or 45-50 mM NaH₂PO₄. In some embodiments, the diluent comprises 0-20, 0.1-20, 0.2-20, 0.3-20, 0.4-20, 0.5-20, 0.6-20, 0.7-20, 0.8-20, 0.9-20, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, or 19-20 mM NaH₂PO₄. In some embodiments, the diluent comprises at least 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mM NaH₂PO₄. In some embodiments, the diluent comprises at most 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mM NaH₂PO₄. In some embodiments, the diluent comprises 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 mM NaH₂PO₄.

In some embodiments, the diluent comprises 0.1-50 mM CaCl₂.

In some embodiments, the diluent comprises 0.1-50, 0.2-50, 0.3-50, 0.4-50, 0.5-50, 0.6-50, 0.7-50, 0.8-50, 0.9-50, 1.0-50, −50, 1.1-50, 1.2-50, 1.3-50, 1.4-50, 1.5-50, 1.6-50, 1.7-50, 1.8-50, 1.9-50, 2.0-50, 2.1-50, 2.2-50, 2.3-50, 2.4-50, 2.5-50, 2.6-50, 2.7-50, 2.8-50, 2.9-50, 3.0-50, 3.1-50, 3.2-50, 3.3-50, 3.4-50, 3.5-50, 3.6-50, 3.7-50, 3.8-50, 3.9-50, 4-50, 5-50, 6-50, 7-50, 8-50, 9-50, 10-50, 15-50, 20-50, 25-50, 1 30-50, 35-50, 40-50, or 45-50 mM CaCl₂. In some embodiments, the diluent comprises 0.1-45, 0.1-40, 0.1-35, 0.1-30, 0.1-25, 0.1-20, 0.1-15, 0.1-10, 0.1-5, 0.1-4, 0.1-4.9, 0.1-4.8, 0.1-4.7, 0.1-4.6, 0.1-4.5, 0.1-4.4, 0.1-4.3, 0.1-4.2, 0.1-4.1, 0.1-4.0, 0.1-3.9, 0.1-3.8, 0.1-3.7, 0.1-3.6, 0.1-3.5, 0.1-3.4, 0.1-3.3, 0.1-3.2, 0.1-3.1, 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM CaCl₂. In some embodiments, the diluent comprises at least 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM CaCl₂. In some embodiments, the diluent comprises at most 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM CaCl₂. In some embodiments, the diluent comprises 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM CaCl₂.

In some embodiments, the diluent comprises 0.1-50 mM MgCl₂.

In some embodiments, the diluent comprises 0.1-50, 0.2-50, 0.3-50, 0.4-50, 0.5-50, 0.6-50, 0.7-50, 0.8-50, 0.9-50, 1.0-50, −50, 1.1-50, 1.2-50, 1.3-50, 1.4-50, 1.5-50, 1.6-50, 1.7-50, 1.8-50, 1.9-50, 2.0-50, 2.1-50, 2.2-50, 2.3-50, 2.4-50, 2.5-50, 2.6-50, 2.7-50, 2.8-50, 2.9-50, 3.0-50, 3.1-50, 3.2-50, 3.3-50, 3.4-50, 3.5-50, 3.6-50, 3.7-50, 3.8-50, 3.9-50, 4-50, 5-50, 6-50, 7-50, 8-50, 9-50, 10-50, 15-50, 20-50, 25-50, 1 30-50, 35-50, 40-50, or 45-50 mM MgCl₂. In some embodiments, the diluent comprises 0.1-45, 0.1-40, 0.1-35, 0.1-30, 0.1-25, 0.1-20, 0.1-15, 0.1-10, 0.1-5, 0.1-4, 0.1-4.9, 0.1-4.8, 0.1-4.7, 0.1-4.6, 0.1-4.5, 0.1-4.4, 0.1-4.3, 0.1-4.2, 0.1-4.1, 0.1-4.0, 0.1-3.9, 0.1-3.8, 0.1-3.7, 0.1-3.6, 0.1-3.5, 0.1-3.4, 0.1-3.3, 0.1-3.2, 0.1-3.1, 0.1-3.0, 0.1-2.9, 0.1-2.8, 0.1-2.7, 0.1-2.6, 0.1-2.5, 0.1-2.4, 0.1-2.3, 0.1-2.2, 0.1-2.1, 0.1-2.0, 0.1-1.9, 0.1-1.8, 0.1-1.7, 0.1-1.6, 0.1-1.5, 0.1-1.4, 0.1-1.3, 0.1-1.2, 0.1-1.1, 0.1-1.0, 0.1-0.9, 0.1-0.8, 0.1-0.7, 0.1-0.6, 0.1-0.5, 0.1-0.4, 0.1-0.3, or 0.1-0.2 mM MgCl₂. In some embodiments the diluent comprises at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM MgCl₂. In some embodiments, the diluent comprises at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM MgCl₂. In some embodiments, the diluent comprises 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 mM MgCl₂.

In some embodiments, the diluent comprises 1-100 mM NaHCO₃, 1-100 mM KHCO₃, or a combination thereof.

In some embodiments, the diluent comprises 1-100 mM NaHCO₃.

In some embodiments, the diluent comprises 1-99, 1-95, 1-90, 1-85, 1-80, 1-75, 1-70, 1-65, 1-60, 1-55, 1-50, 1-45, 1-40, 1-35, 1-30, 1-25, 1-20, 1-15, 1-10, 1-9, 1-8, 1-7, 1-6, 1-5, 1-4, 1-3, or 1-2 mM NaHCO₃. In some embodiments, the diluent comprises 2-100, 3-100, 4-100, 5-100, 6-100, 7-100, 8-100, 9-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100 mM NaHCO₃. In some embodiments, the diluent comprises 24.0-28.0, 24.0-27.9, 24.0-27.8, 24.0-27.7, 24.0-27.6, 24.0-27.5, 24.0-27.4, 24.0-27.3, 24.0-27.2, 24.0-27.1, 24.0-27.0, 24.0-26.9, 24.0-26.8, 24.0-26.7, 24.0-26.6, 24.0-26.5, 24.0-26.4, 24.0-26.3, 24.0-26.2, 24.0-26.1, 24.0-26.0, 24.0-25.9, 4.0-25.8, 24.0-25.7, 24.0-25.6, 24.0-25.5, 24.0-25.4, 24.0-25.3, 24.0-25.2, 24.0-25.1, 24.0-25.0, 24.0-24.9, 24.0-24.8, 24.0-24.7, 24.0-24.6, 24.0-24.5, 24.0-24.4, 24.0-24.3, 24.0-24.2, or 24.0-24.1 mM NaHCO₃. In some embodiments, the diluent comprises 24.1-28.0, 24.2-28.0, 24.3-28.0, 24.4-28.0, 24.5-28.0, 24.6-28.0, 24.7-28.0, 24.8-28.0, 24.9-28.0, 25.0-28.0, 25.1-28.0, 25.2-28.0, 25.3-28.0, 25.4-28.0, 25.5-28.0, 25.6-28.0, 25.7-28.0, 25.8-28.0, 25.9-28.0, 26.0-28.0, 26.1-28.0, 26.2-28.0, 26.3-28.0, 26.4-28.0, 26.5-28.0, 26.6-28.0, 26.7-28.0, 26.8-28.0, 26.9-28.0, 27.0-28.0, 27.1-28.0, 27.2-28.0, 27.3-28.0, 27.4-28.0, 27.5-28.0, 27.6-28.0, 27.7-28.0, 27.8-28.0, or 27.9-28.0 mM NaHCO₃. In some embodiments, the diluent comprises at least 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM NaHCO₃. In some embodiments, the diluent comprises at most 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM NaHCO₃. In some embodiments, the diluent comprises 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM NaHCO₃.

In some embodiments, the diluent comprises 1-100 mM KHCO₃.

In some embodiments, the diluent comprises 1-99, 1-95, 1-90, 1-85, 1-80, 1-75, 1-70, 1-65, 1-60, 1-55, 1-50, 1-45, 1-40, 1-35, 1-30, 1-25, 1-20, 1-15, 1-10, 1-9, 1-8, 1-7, 1-6, 1-5, 1-4, 1-3, or 1-2 mM KHCO₃. In some embodiments, the diluent comprises 2-100, 3-100, 4-100, 5-100, 6-100, 7-100, 8-100, 9-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100 mM KHCO₃. In some embodiments, the diluent comprises 24.0-28.0, 24.0-27.9, 24.0-27.8, 24.0-27.7, 24.0-27.6, 24.0-27.5, 24.0-27.4, 24.0-27.3, 24.0-27.2, 24.0-27.1, 24.0-27.0, 24.0-26.9, 24.0-26.8, 24.0-26.7, 24.0-26.6, 24.0-26.5, 24.0-26.4, 24.0-26.3, 24.0-26.2, 24.0-26.1, 24.0-26.0, 24.0-25.9, 4.0-25.8, 24.0-25.7, 24.0-25.6, 24.0-25.5, 24.0-25.4, 24.0-25.3, 24.0-25.2, 24.0-25.1, 24.0-25.0, 24.0-24.9, 24.0-24.8, 24.0-24.7, 24.0-24.6, 24.0-24.5, 24.0-24.4, 24.0-24.3, 24.0-24.2, or 24.0-24.1 mM KHCO₃. In some embodiments, the diluent comprises 24.1-28.0, 24.2-28.0, 24.3-28.0, 24.4-28.0, 24.5-28.0, 24.6-28.0, 24.7-28.0, 24.8-28.0, 24.9-28.0, 25.0-28.0, 25.1-28.0, 25.2-28.0, 25.3-28.0, 25.4-28.0, 25.5-28.0, 25.6-28.0, 25.7-28.0, 25.8-28.0, 25.9-28.0, 26.0-28.0, 26.1-28.0, 26.2-28.0, 26.3-28.0, 26.4-28.0, 26.5-28.0, 26.6-28.0, 26.7-28.0, 26.8-28.0, 26.9-28.0, 27.0-28.0, 27.1-28.0, 27.2-28.0, 27.3-28.0, 27.4-28.0, 27.5-28.0, 27.6-28.0, 27.7-28.0, 27.8-28.0, or 27.9-28.0 mM KHCO₃. In some embodiments, the diluent comprises at least 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.0, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM KHCO₃. In some embodiments, the diluent comprises at most 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM KHCO₃. In some embodiments, the diluent comprises 24.0, 24.1, 24.2, 24.3, 24.4, 24.5, 24.6, 24.7, 24.8, 24.9, 25.0, 25.1, 25.2, 25.3, 25.4, 25.5, 25.6, 25.7, 25.8, 25.9, 26.1, 26.2, 26.3, 26.4, 26.5, 26.6, 26.7, 26.8, 26.9, 27.0, 27.1, 27.2, 27.3, 27.4, 27.5, 27.6, 27.7, 27.8, 27.9, or 28.0 mM KHCO₃.

In some embodiments, the diluent comprises 1-100 mM D-glucose.

In some embodiments, the diluent comprises 1-100, 1-95, 1-90, 1-85, 1-80, 1-75, 1-70, 1-65, 1-60, 1-55, 1-50, 1-45, 1-40, 1-35, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 mM D-glucose. In some embodiments, the diluent comprises 2-100, 3-100, 4-100, 5-100, 6-100, 7-100, 8-100, 9-100, 10-100, 11-100, 12-100, 13-100, 14-100, 15-100, 16-100, 17-100, 18-100, 19-100, 20-100, 21-100, 22-100, 23-100, 24-100, 25-100, 26-100, 29-100, 28-100, 29-100, 30-100, 35-100, 40-100, 45-100, 50-100, 55-100, 60-100, 65-100, 70-100, 75-100, 80-100, 85-100, 90-100, or 95-100 mM D-glucose. In some embodiments, the diluent comprises 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 mM D-glucose. In some embodiments, the diluent comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM D-glucose. In some embodiments, the diluent comprises at most 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM D-glucose. In some embodiments, the diluent comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM D-glucose.

In some embodiments, the diluent comprises 1-100, 1-95, 1-90, 1-85, 1-80, 1-75, 1-70, 1-65, 1-60, 1-55, 1-50, 1-45, 1-40, 1-35, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, or 1-2 mM glucose. In some embodiments, the diluent comprises 2-100, 3-100, 4-100, 5-100, 6-100, 7-100, 8-100, 9-100, 10-100, 11-100, 12-100, 13-100, 14-100, 15-100, 16-100, 17-100, 18-100, 19-100, 20-100, 21-100, 22-100, 23-100, 24-100, 25-100, 26-100, 29-100, 28-100, 29-100, 30-100, 35-100, 40-100, 45-100, 50-100, 55-100, 60-100, 65-100, 70-100, 75-100, 80-100, 85-100, 90-100, or 95-100 mM glucose. In some embodiments, the diluent comprises 2-30, 3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, or 29-30 mM glucose. In some embodiments, the diluent comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM glucose. In some embodiments, the diluent comprises at most 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM glucose. In some embodiments, the diluent comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mM glucose.

In some embodiments, the diluent comprises 25-250 mM NaCl, 0.1-20 mM KCl, 0.1-50 mM Na₂HPO₄, 0.1-50 mM NaH₂PO₄, 0.1-50 mM CaCl₂, and 0.1-50 mM MgCl₂.

In some embodiments, the diluent comprises 150 mM NaCl, 3.0 mM KCl, 0.7 mM Na₂HPO₄, 0.3 mM NaH₂PO₄, 0.79 mM MgCl₂, and 1.4 mM CaCl₂.

In some embodiments, the diluent further comprises an antioxidant. In some embodiments, the antioxidant is t-butylhydroxyquinoline (TBHQ), butylated hydroxytoluene (BHT), butylated hydroxyanisole (BHA), vitamin E, or any combination thereof. In some embodiments, the compound as described herein (e.g., compound (I) or a salt thereof, or compound (II)) is solubilized or diluted in a buffer further comprising an antioxidant, wherein the antioxidant is ascorbic acid (vitamin C), glutathione, lipoic acid, uric acid, carotenes, α-tocopherol (vitamin E), ubiquinol (coenzyme Q), or any combination thereof.

In some embodiments, the diluent comprises 25-250 mM NaCl, 0.1-20 mM KCl, 0-50 mM KH₂PO₄, 1-100 mM NaHCO₃, 0-50 mM NaH₂PO₄, 1-100 mM D-glucose, 0.1-50 mM CaCl₂, 0.1-50 mM MgCl₂, or any combinations thereof.

In some embodiments, the diluent comprises 25-250 mM NaCl, 0.1-20 mM KCl, 0-50 mM KH₂PO₄, 1-100 mM NaHCO₃, 0-50 mM NaH₂PO₄, 1-100 mM D-glucose, 0.1-50 mM CaCl₂, and 0.1-50 mM MgCl₂.

In some embodiments, the diluent comprises 127 mM NaCl, 1.0 mM KCl, 1.2 mM KH₂PO₄, 26 mM NaHCO₃, 10 mM D-glucose, 2.4 mM CaCl₂, and 1.3 mM MgCl₂.

In some embodiments, the diluent comprises 119 mM NaCl, 26.2 mM NaHCO₃, 2.5 mM KCl, 1 mM NaH₂PO₄, 1.3 mM MgCl₂, 10 mM glucose, and 2.5 mM CaCl₂.

In some embodiments, the diluent does not comprise a preservative. In some embodiments, the diluent comprises a preservative.

In some embodiments, the instructions for diluting or solubilizing the compound as described herein in the diluent comprise instructions for diluting or solubilizing the compound as described herein to a concentration of from 5-200 mg/mL in the diluent.

In some embodiments, the instructions for diluting or solubilizing the compound as described herein in the diluent comprise instructions for diluting or solubilizing the compound as described herein to a concentration of from 5-250, 5-247.5, 5-245, 5-242.5, 5-240, 5-237.5, 5-235, 5-232.5, 5-230, 5-227.5, 5-225, 5-225.5, 5-220, 5-217.5, 5-215, 5-212.5, 5-210, 5-205.5, 5-205, 5-202.5, 5-200, 5-197.5, 5-195, 5-192.5, 5-190, 5-187.5, 5-185, 5-182.5, 5-180, 5-177.5, 5-175, 5-172.5, 5-170, 5-167.5, 5-165, 5-162.5, 5-160, 5-157.5, 5-155, 5-152.5, 5-150, 5-147.5, 5-145, 5-142.5, 5-140, 5-137.5, 5-135, 5-132.5, 5-130, 5-127.5, 5-125, 5-122.5, 5-120, 5-117.5, 5-115, 5-112.5, 5-110, 5-107.5, 5-105, 5-102.5, 5-100, 5-97.5, 5-95, 5-92.5, 5-90, 5-87.5, 5-85, 5-82.5, 5-80, 5-77.5, 5-75, 5-72.5, 5-70, 5-67.5, 5-65, 5-62.5, 5-60, 5-57.5, 5-55, 5-52.5, 5-50, 5-47.5, 5-45, 5-42.5, 5-40, 5-37.5, 5-35, 5-32.5, 5-30, 5-27.5, 5-25, 5-22.5, 5-20, 5-17.5, 5-15, 5-12.5, or 5-10 mg/mL in the diluent. In some embodiments, the instructions for diluting or solubilizing the compound as described herein in the diluent comprise instructions for diluting or solubilizing the compound as described herein to a concentration of from 10-250, 15-250, 20-250, 25-250, 30-250, 35-250, 40-250, 45-250, 50-250, 55-250, 60-250, 65-250, 70-250, 75-250, 80-250, 85-250, 90-250, 95-250, 100-250, 105-250, 110-250, 115-250, 120-250, 125-250, 130-250, 135-250, 140-250, 145-250, 150-250, 155-250, 160-250, 165-250, 170-250, 175-250, 180-250, 185-250, 190-250, or 195-250, 200-250, 205-250, 210-250, 215-250, 220-250, 225-250, 230-250, 235-250, 240-250, or 245-250 mg/mL in the diluent. In some embodiments, the instructions for diluting or solubilizing the compound as described herein in the diluent comprise instructions for diluting or solubilizing the compound as described herein to a concentration of from at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent. In some embodiments, the instructions for diluting or solubilizing the compound as described herein in the diluent comprise instructions for diluting or solubilizing the compound as described herein to a concentration of from at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent. In some embodiments, the instructions for diluting or solubilizing the compound as described herein in the diluent comprise instructions for diluting or solubilizing the compound as described herein to a concentration of from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent.

In some embodiments, the compound as described herein (e.g., compound (I) or a salt thereof, or compound (II)) is present in the pharmaceutical composition at a concentration of the compound as described herein (e.g., compound (I) or a salt thereof, or compound (II)) is present in the pharmaceutical composition at a concentration of from 0.1 mg/mL to 250 mg/mL. In some embodiments, the compound as described herein (e.g., compound (I) or a salt thereof, or compound (II)) is present in the pharmaceutical composition at a concentration of about 0.1 mg/mL, 0.5 mg/mL, 1 mg/mL, 2 mg/mL, 2.5 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, or 20 mg/mL. In some embodiments, the compound as described herein (e.g., compound (I) or a salt thereof, or compound (II)) is present in the pharmaceutical composition at a concentration of about 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL. In some embodiments, the compound as described herein (e.g., compound (I) or a salt thereof, or compound (II)) is present in the pharmaceutical composition at a concentration of about 22.5 mg/mL, 25 mg/mL, 27.5 mg/mL, 30 mg/mL, 32.5 mg/mL, 35 mg/mL, 37.5 mg/mL, 40 mg/mL, 42.5 mg/mL, 45 mg/mL, 47.5 mg/mL, 50 mg/mL, 52.5 mg/mL, 55 mg/mL, 57.5 mg/mL, 60 mg/mL, 62.5 mg/mL, 65 mg/mL, 67.5 mg/mL, 70 mg/mL, 72.5 mg/mL, 75 mg/mL, 77.5 mg/mL, 80 mg/mL, 82.5 mg/mL, 85 mg/mL, 87.5 mg/mL, 90 mg/mL, 92.5 mg/mL, 95 mg/mL, 97.5 mg/mL, 100 mg/mL, 102.5 mg/mL, 105 mg/mL, 107.5 mg/mL, 110 mg/mL, 112.5 mg/mL, 115 mg/mL, 117.5 mg/mL, 120 mg/mL, 122.5 mg/mL, 125 mg/mL, 127.5 mg/mL, 130 mg/mL, 132.5 mg/mL, 135 mg/mL, 137.5 mg/mL, 140 mg/mL, 142.5 mg/mL, 145 mg/mL, 147.5 mg/mL, 150 mg/mL, 152.5 mg/mL, 155 mg/mL, 157.5 mg/mL, 160 mg/mL, 162.5 mg/mL, 165 mg/mL, 167.5 mg/mL, 170 mg/mL, 172.5 mg/mL, 175 mg/mL, 177.5 mg/mL, 180 mg/mL, 182.5 mg/mL, 185 mg/mL, 187.5 mg/mL, 190 mg/mL, 192.5 mg/mL, 195 mg/mL, 197.5 mg/mL, 200 mg/mL, 202.5 mg/mL, 205 mg/mL, 207.5 mg/mL, 210 mg/mL, 212.5 mg/mL, 215 mg/mL, 217.5 mg/mL, 220 mg/mL, 222.5 mg/mL, 225 mg/mL, 227.5 mg/mL, 230 mg/mL, 232.5 mg/mL, 235 mg/mL, 237.5 mg/mL, 240 mg/mL, 242.5 mg/mL, 245 mg/mL, 247.5 mg/mL, or 250 mg/mL. In some embodiments, the compound as described herein (e.g., compound (I) or a salt thereof, or compound (II)) is solubilized or diluted to a concentration of 11 mg/mL, 22 mg/mL, 33 mg/mL, 44 mg/mL, 55 mg/mL, 66 mg/mL, 77 mg/mL, 88 mg/mL, 99 mg/mL, or 100 mg/mL in the diluent.

In some embodiments, the compound as described herein (e.g., compound (I) or a salt thereof, or compound (II)) is present in the pharmaceutical composition at a concentration of from 0.1 mg/mL to 250 mg/mL, from 0.2 mg/mL to 250 mg/mL, from 0.3 mg/mL to 250 mg/mL, from 0.4 mg/mL to 250 mg/mL, from 0.5 mg/mL to 250 mg/mL, from 0.6 mg/mL to 250 mg/mL, from 0.7 mg/mL to 250 mg/mL, from 0.8 mg/mL to 250 mg/mL, from 0.9 mg/mL to 250 mg/mL, from 1.0 mg/mL to 250 mg/mL, from 1.1 mg/mL to 250 mg/mL, from 1.2 mg/mL to 250 mg/mL, from 1.3 mg/mL to 250 mg/mL, from 1.4 mg/mL to 250 mg/mL, from 1.5 mg/mL to 250 mg/mL, from 1.6 mg/mL to 250 mg/mL, from 1.7 mg/mL to 250 mg/mL, from 1.8 mg/mL to 250 mg/mL, from 1.9 mg/mL to 250 mg/mL, from 2.0 mg/mL to 250 mg/mL, from 2.1 mg/mL to 250 mg/mL, from 2.2 mg/mL to 250 mg/mL, from 2.3 mg/mL to 250 mg/mL, from 2.4 mg/mL to 250 mg/mL, from 2.5 mg/mL to 250 mg/mL, from 2.6 mg/mL to 250 mg/mL, from 2.7 mg/mL to 250 mg/mL, from 2.8 mg/mL to 250 mg/mL, from 2.9 mg/mL to 250 mg/mL, from 3.0 mg/mL to 250 mg/mL, from 3.1 mg/mL to 250 mg/mL, from 3.2 mg/mL to 250 mg/mL, from 3.3 mg/mL to 250 mg/mL, from 3.4 mg/mL to 250 mg/mL, from 3.5 mg/mL to 250 mg/mL, from 3.6 mg/mL to 250 mg/mL, from 3.7 mg/mL to 250 mg/mL, from 3.8 mg/mL to 250 mg/mL, from 3.9 mg/mL to 250 mg/mL, from 4.0 mg/mL to 250 mg/mL, from 5.0 mg/mL to 250 mg/mL, from 6.0 mg/mL to 250 mg/mL, from 7.0 mg/mL to 250 mg/mL, from 8.0 mg/mL to 250 mg/mL, from 9.0 mg/mL to 250 mg/mL, from 10 mg/mL to 250 mg/mL, from 15 mg/mL to 250 mg/mL, from 20 mg/mL to 250 mg/mL, from 25 mg/mL to 250 mg/mL, from 30 mg/mL to 250 mg/mL, from 35 mg/mL to 250 mg/mL, from 40 mg/mL to 250 mg/mL, from 45 mg/mL to 250 mg/mL, from 50 mg/mL to 250 mg/mL, from 55 mg/mL to 250 mg/mL, from 60 mg/mL to 250 mg/mL, from 65 mg/mL to 250 mg/mL, from 70 mg/mL to 250 mg/mL, from 75 mg/mL to 250 mg/mL, from 80 mg/mL to 250 mg/mL, from 85 mg/mL to 250 mg/mL, from 90 mg/mL to 250 mg/mL, from 95 mg/mL to 250 mg/mL, from 100 mg/mL to 250 mg/mL, from 105 mg/mL to 250 mg/mL, from 110 mg/mL to 250 mg/mL, from 115 mg/mL to 250 mg/mL, from 120 mg/mL to 250 mg/mL, from 125 mg/mL to 250 mg/mL, from 130 mg/mL to 250 mg/mL, from 135 mg/mL to 250 mg/mL, from 140 mg/mL to 250 mg/mL, from 145 mg/mL to 250 mg/mL, from 150 mg/mL to 250 mg/mL, from 155 mg/mL to 250 mg/mL, from 160 mg/mL to 250 mg/mL, from 165 mg/mL to 250 mg/mL, from 170 mg/mL to 250 mg/mL, from 175 mg/mL to 250 mg/mL, from 180 mg/mL to 250 mg/mL, from 185 mg/mL to 250 mg/mL, from 190 mg/mL to 250 mg/mL, from 195 mg/mL to 250 mg/mL, 200 mg/mL to 250 mg/mL, from 205 mg/mL to 250 mg/mL, from 210 mg/mL to 250 mg/mL, from 215 mg/mL to 250 mg/mL, from 220 mg/mL to 250 mg/mL, from 225 mg/mL to 250 mg/mL, from 230 mg/mL to 250 mg/mL, from 235 mg/mL to 250 mg/mL, from 240 mg/mL to 250 mg/mL, or from 245 mg/mL to 250 mg/mL.

In some embodiments, the compound as described herein (e.g., compound (I) or a salt thereof, or compound (II)) is present in the pharmaceutical composition at a concentration of from 0.1 mg/mL to 250 mg/mL, from 0.1 mg/mL to 245 mg/mL, from 0.1 mg/mL to 240 mg/mL, from 0.1 mg/mL to 235 mg/mL, from 0.1 mg/mL to 230 mg/mL, from 0.1 mg/mL to 225 mg/mL, from 0.1 mg/mL to 220 mg/mL, from 0.1 mg/mL to 215 mg/mL, from 0.1 mg/ml to 210 mg/mL, from 0.1 mg/mL to 205 mg/mL, from 0.1 mg/mL to 200 mg/mL, from 0.1 mg/mL to 195 mg/mL, from 0.1 mg/mL to 190 mg/mL, from 0.1 mg/mL to 185 mg/mL, from 0.1 mg/mL to 180 mg/mL, from 0.1 mg/mL to 175 mg/mL, from 0.1 mg/mL to 170 mg/mL, from 0.1 mg/mL to 165 mg/mL, from 0.1 mg/mL to 160 mg/mL, from 0.1 mg/mL to 155 mg/mL, from 0.1 mg/mL to 150 mg/mL, from 0.1 mg/mL to 145 mg/mL, from 0.1 mg/mL to 140 mg/mL, from 0.1 mg/mL to 135 mg/mL, from 0.1 mg/mL to 130 mg/mL, from 0.1 mg/mL to 125 mg/mL, from 0.1 mg/mL to 120 mg/mL, from 0.1 mg/mL to 115 mg/mL, from 0.1 mg/mL to 110 mg/mL, from 0.1 mg/mL to 100 mg/mL, from 0.1 mg/mL to 95 mg/mL, from 0.1 mg/mL to 90 mg/mL, from 0.1 mg/mL to 85 mg/mL, from 0.1 mg/mL to 80 mg/mL, from 0.1 mg/mL to 75 mg/mL, from 0.1 mg/mL to 70 mg/mL, from 0.1 mg/mL to 65 mg/mL, from 0.1 mg/mL to 60 mg/mL, from 0.1 mg/mL to 55 mg/mL, from 0.1 mg/mL to 50 mg/mL, from 0.1 mg/mL to 45 mg/mL, from 0.1 mg/mL to 40 mg/mL, from 0.1 mg/mL to 35 mg/mL, from 0.1 mg/mL to 30 mg/mL, from 0.1 mg/mL to 25 mg/mL, from 0.1 mg/mL to 20 mg/mL, from 0.1 mg/mL to 15 mg/mL, from 0.1 mg/mL to 10 mg/mL, from 0.1 mg/mL to 9 mg/mL, from 0.1 mg/mL to 8 mg/mL, from 0.1 mg/mL to 7 mg/mL, from 0.1 mg/mL to 6 mg/mL, from 0.1 mg/mL to 5 mg/mL, from 0.1 mg/mL to 4 mg/mL, from 0.1 mg/mL to 3.9 mg/mL, from 0.1 mg/mL to 3.8 mg/mL, from 0.1 mg/mL to 3.7 mg/mL, from 0.1 mg/mL to 3.6 mg/mL, from 0.1 mg/mL to 3.5 mg/mL, from 0.1 mg/mL to 3.4 mg/mL, from 0.1 mg/mL to 3.3 mg/mL, from 0.1 mg/mL to 3.2 mg/mL, from 0.1 mg/mL to 3.1 mg/mL, from 0.1 mg/mL to 3.0 mg/mL, from 0.1 mg/mL to 2.9 mg/mL, from 0.1 mg/mL to 2.8 mg/mL, from 0.1 mg/mL to 2.7 mg/mL, from 0.1 mg/mL to 2.6 mg/mL, from 0.1 mg/mL to 2.5 mg/mL, from 0.1 mg/mL to 2.4 mg/mL, from 0.1 mg/mL to 2.3 mg/mL, from 0.1 mg/mL to 2.2 mg/mL, from 0.1 mg/mL to 2.1 mg/mL, from 0.1 mg/mL to 2.0 mg/mL, from 0.1 mg/mL to 1.9 mg/mL, from 0.1 mg/mL to 1.8 mg/mL, from 0.1 mg/mL to 1.7 mg/mL, from 0.1 mg/mL to 1.6 mg/mL, from 0.1 mg/mL to 1.5 mg/mL, from 0.1 mg/mL to 1.4 mg/mL, from 0.1 mg/mL to 1.3 mg/mL, from 0.1 mg/mL to 1.2 mg/mL, from 0.1 mg/mL to 1.1 mg/mL, from 0.1 mg/mL to 1.0 mg/mL, from 0.1 mg/mL to 0.9 mg/mL, from 0.1 mg/mL to 0.8 mg/mL, from 0.1 mg/mL to 0.7 mg/mL, from 0.1 mg/mL to 0.6 mg/mL, from 0.1 mg/mL to 0.5 mg/mL, from 0.1 mg/mL to 0.4 mg/mL, from 0.1 mg/mL to 0.3 mg/mL, or from 0.1 mg/mL to 0.2 mg/mL.

In some embodiments, the compound as described herein (e.g., compound (I) or a salt thereof, or compound (II)) is solubilized or diluted to a concentration of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent. In some embodiments, the compound as described herein (e.g., compound (I) or a salt thereof, or compound (II)) is solubilized or diluted to a concentration of at most 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent. In some embodiments, the compound as described herein (e.g., compound (I) or a salt thereof, or compound (II)) is solubilized or diluted to a concentration of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 mg/mL in the diluent.

In some embodiments, the instructions for diluting or solubilizing the compound as described herein in the diluent comprise instructions for diluting or solubilizing the compound as described herein to a concentration of from 0.1 mg/mL to 200 mg/mL. In some embodiments, the instructions for diluting or solubilizing the compound as described herein in the diluent comprise instructions for diluting or solubilizing the compound as described herein to a concentration of about 0.1 mg/mL, 0.5 mg/mL, 1 mg/mL, 2 mg/mL, 2.5 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, or 20 mg/mL. In some embodiments, the instructions for diluting or solubilizing the compound as described herein in the diluent comprise instructions for diluting or solubilizing the compound as described herein to a concentration of about 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL. In some embodiments, the instructions for diluting or solubilizing the compound as described herein in the diluent comprise instructions for diluting or solubilizing the compound as described herein to a concentration of 11 mg/mL, 22 mg/mL, 33 mg/mL, 44 mg/mL, 55 mg/mL, 66 mg/mL, 77 mg/mL, 88 mg/mL, 99 mg/mL, or 100 mg/mL in the diluent.

In some embodiments, the instructions for diluting or solubilizing the compound as described herein in the diluent comprise instructions for diluting or solubilizing the compound as described herein to a concentration of from 0.1 mg/mL to 200 mg/mL, from 0.2 mg/mL to 200 mg/mL, from 0.3 mg/mL to 200 mg/mL, from 0.4 mg/mL to 200 mg/mL, from 0.5 mg/mL to 200 mg/mL, from 0.6 mg/mL to 200 mg/mL, from 0.7 mg/mL to 200 mg/mL, from 0.8 mg/mL to 200 mg/mL, from 0.9 mg/mL to 200 mg/mL, from 1.0 mg/mL to 200 mg/mL, from 1.1 mg/mL to 200 mg/mL, from 1.2 mg/mL to 200 mg/mL, from 1.3 mg/mL to 200 mg/mL, from 1.4 mg/mL to 200 mg/mL, from 1.5 mg/mL to 200 mg/mL, from 1.6 mg/mL to 200 mg/mL, from 1.7 mg/mL to 200 mg/mL, from 1.8 mg/mL to 200 mg/mL, from 1.9 mg/mL to 200 mg/mL, from 2.0 mg/mL to 200 mg/mL, from 2.1 mg/mL to 200 mg/mL, from 2.2 mg/mL to 200 mg/mL, from 2.3 mg/mL to 200 mg/mL, from 2.4 mg/mL to 200 mg/mL, from 2.5 mg/mL to 200 mg/mL, from 2.6 mg/mL to 200 mg/mL, from 2.7 mg/mL to 200 mg/mL, from 2.8 mg/mL to 200 mg/mL, from 2.9 mg/mL to 200 mg/mL, from 3.0 mg/mL to 200 mg/mL, from 3.1 mg/mL to 200 mg/mL, from 3.2 mg/mL to 200 mg/mL, from 3.3 mg/mL to 200 mg/mL, from 3.4 mg/mL to 200 mg/mL, from 3.5 mg/mL to 200 mg/mL, from 3.6 mg/mL to 200 mg/mL, from 3.7 mg/mL to 200 mg/mL, from 3.8 mg/mL to 200 mg/mL, from 3.9 mg/mL to 200 mg/mL, from 4.0 mg/mL to 200 mg/mL, from 5.0 mg/mL to 200 mg/mL, from 6.0 mg/ml to 200 mg/mL, from 7.0 mg/mL to 200 mg/mL, from 8.0 mg/mL to 200 mg/mL, from 9.0 mg/mL to 200 mg/mL, from 10 mg/mL to 200 mg/mL, from 15 mg/mL to 200 mg/mL, from 20 mg/mL to 200 mg/mL, from 25 mg/mL to 200 mg/mL, from 30 mg/mL to 200 mg/mL, from 35 mg/mL to 200 mg/mL, from 40 mg/mL to 200 mg/mL, from 45 mg/mL to 200 mg/mL, from 50 mg/mL to 200 mg/mL, from 55 mg/mL to 200 mg/mL, from 60 mg/mL to 200 mg/mL, from 65 mg/mL to 200 mg/mL, from 70 mg/mL to 200 mg/mL, from 75 mg/mL to 200 mg/mL, from 80 mg/mL to 200 mg/mL, from 85 mg/mL to 200 mg/mL, from 90 mg/mL to 200 mg/mL, from 95 mg/mL to 200 mg/mL, from 100 mg/mL to 200 mg/mL, from 105 mg/mL to 200 mg/mL, from 110 mg/mL to 200 mg/mL, from 115 mg/mL to 200 mg/mL, from 120 mg/mL to 200 mg/mL, from 125 mg/mL to 200 mg/mL, from 130 mg/mL to 200 mg/mL, from 135 mg/mL to 200 mg/mL, from 140 mg/mL to 200 mg/mL, from 145 mg/mL to 200 mg/mL, from 150 mg/mL to 200 mg/mL, from 155 mg/mL to 200 mg/mL, from 160 mg/mL to 200 mg/mL, from 165 mg/mL to 200 mg/mL, from 170 mg/mL to 200 mg/mL, from 175 mg/mL to 200 mg/mL, from 180 mg/mL to 200 mg/mL, from 185 mg/mL to 200 mg/mL, from 190 mg/mL to 200 mg/mL, or from 195 mg/mL to 200 mg/mL.

In some embodiments, the instructions for diluting or solubilizing the compound as described herein in the diluent comprise instructions for diluting or solubilizing the compound as described herein to a concentration of from 0.1 mg/mL to 200 mg/mL, from 0.1 mg/mL to 195 mg/mL, from 0.1 mg/mL to 190 mg/mL, from 0.1 mg/mL to 185 mg/mL, from 0.1 mg/mL to 180 mg/mL, from 0.1 mg/mL to 175 mg/mL, from 0.1 mg/mL to 170 mg/mL, from 0.1 mg/mL to 165 mg/mL, from 0.1 mg/mL to 160 mg/mL, from 0.1 mg/mL to 155 mg/mL, from 0.1 mg/mL to 150 mg/mL, from 0.1 mg/mL to 145 mg/mL, from 0.1 mg/mL to 140 mg/mL, from 0.1 mg/mL to 135 mg/mL, from 0.1 mg/mL to 130 mg/mL, from 0.1 mg/mL to 125 mg/mL, from 0.1 mg/mL to 120 mg/mL, from 0.1 mg/mL to 115 mg/mL, from 0.1 mg/mL to 110 mg/mL, from 0.1 mg/mL to 100 mg/mL, from 0.1 mg/mL to 95 mg/mL, from 0.1 mg/mL to 90 mg/mL, from 0.1 mg/mL to 85 mg/mL, from 0.1 mg/mL to 80 mg/mL, from 0.1 mg/mL to 75 mg/mL, from 0.1 mg/mL to 70 mg/mL, from 0.1 mg/mL to 65 mg/mL, from 0.1 mg/mL to 60 mg/mL, from 0.1 mg/mL to 55 mg/mL, from 0.1 mg/mL to 50 mg/mL, from 0.1 mg/mL to 45 mg/mL, from 0.1 mg/mL to 40 mg/mL, from 0.1 mg/mL to 35 mg/mL, from 0.1 mg/mL to 30 mg/mL, from 0.1 mg/mL to 25 mg/mL, from 0.1 mg/mL to 20 mg/mL, from 0.1 mg/mL to 15 mg/mL, from 0.1 mg/mL to 10 mg/mL, from 0.1 mg/mL to 9 mg/mL, from 0.1 mg/mL to 8 mg/mL, from 0.1 mg/mL to 7 mg/mL, from 0.1 mg/mL to 6 mg/mL, from 0.1 mg/mL to 5 mg/mL, from 0.1 mg/mL to 4 mg/mL, from 0.1 mg/mL to 3.9 mg/mL, from 0.1 mg/mL to 3.8 mg/mL, from 0.1 mg/mL to 3.7 mg/mL, from 0.1 mg/mL to 3.6 mg/mL, from 0.1 mg/mL to 3.5 mg/mL, from 0.1 mg/mL to 3.4 mg/mL, from 0.1 mg/mL to 3.3 mg/mL, from 0.1 mg/mL to 3.2 mg/mL, from 0.1 mg/mL to 3.1 mg/mL, from 0.1 mg/mL to 3.0 mg/mL, from 0.1 mg/mL to 2.9 mg/mL, from 0.1 mg/mL to 2.8 mg/mL, from 0.1 mg/mL to 2.7 mg/mL, from 0.1 mg/mL to 2.6 mg/mL, from 0.1 mg/mL to 2.5 mg/mL, from 0.1 mg/mL to 2.4 mg/mL, from 0.1 mg/mL to 2.3 mg/mL, from 0.1 mg/mL to 2.2 mg/mL, from 0.1 mg/mL to 2.1 mg/mL, from 0.1 mg/mL to 2.0 mg/mL, from 0.1 mg/mL to 1.9 mg/mL, from 0.1 mg/mL to 1.8 mg/mL, from 0.1 mg/mL to 1.7 mg/mL, from 0.1 mg/mL to 1.6 mg/mL, from 0.1 mg/mL to 1.5 mg/mL, from 0.1 mg/mL to 1.4 mg/mL, from 0.1 mg/mL to 1.3 mg/mL, from 0.1 mg/mL to 1.2 mg/mL, from 0.1 mg/mL to 1.1 mg/mL, from 0.1 mg/mL to 1.0 mg/mL, from 0.1 mg/mL to 0.9 mg/mL, from 0.1 mg/mL to 0.8 mg/mL, from 0.1 mg/mL to 0.7 mg/mL, from 0.1 mg/mL to 0.6 mg/mL, from 0.1 mg/mL to 0.5 mg/mL, from 0.1 mg/mL to 0.4 mg/mL, from 0.1 mg/mL to 0.3 mg/mL, or from 0.1 mg/mL to 0.2 mg/mL.

One aspect of the disclosure relates to kits including the compound as described herein. The kits can further include one or more additional therapeutic regimens or agents.

Also disclosed herein, in some embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include the composition of the invention, and optionally in addition with therapeutic regimens or agents disclosed herein. Such kits optionally include an identifying description or label or instructions relating to its use in the methods described herein.

A kit typically includes labels listing contents and/or instructions for use, and package inserts with instructions for use. A set of instructions will also typically be included.

In some embodiments, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### EXAMPLES

The present disclosure will be more specifically illustrated by the following Examples. However, it should be understood that the present disclosure is not limited by these examples in any manner.

### Example 1: Mechanism of TANGO (Targeted Augmentation of Nuclear Gene Output)

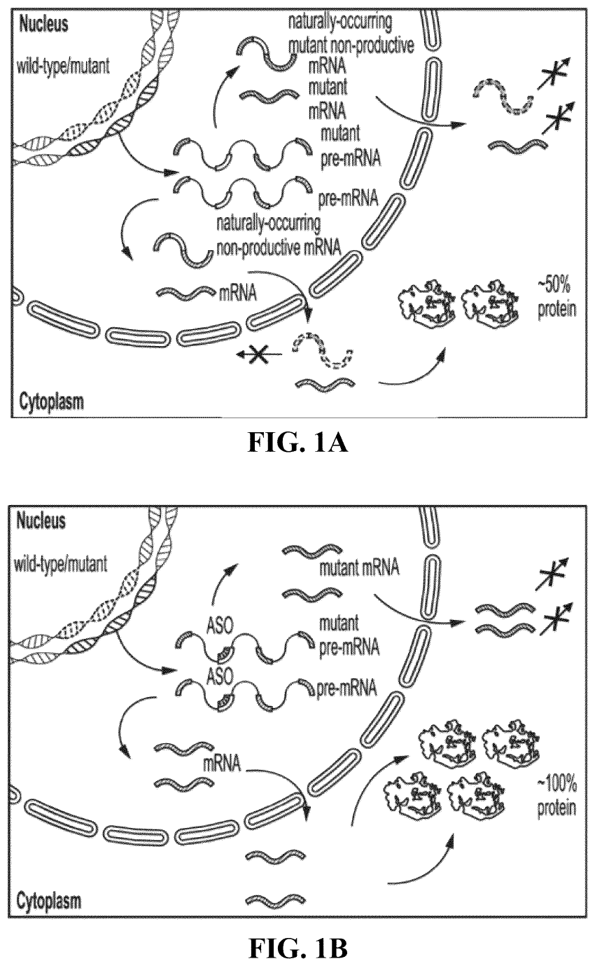

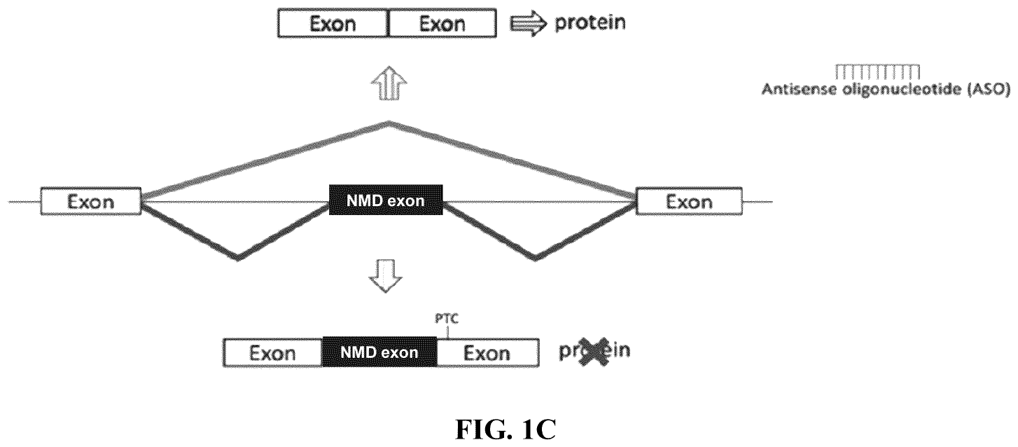

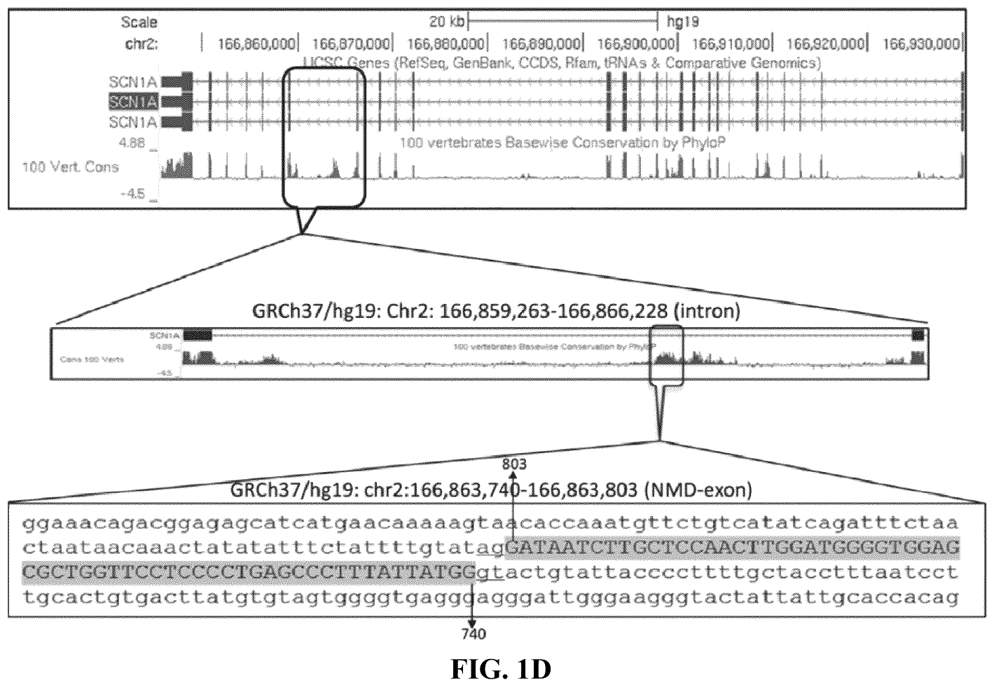

TANGO uses ASOs to specifically increase protein expression by targeting naturally occurring non-productive alternative splicing events. TANGO can reduce non-productive messenger RNAs (mRNA), which are normally targeted for degradation by nonsense-mediated mRNA decay (NMD) as shown in FIGS. 1A and 1B. In turn, TANGO can increase productive mRNA and protein. TANGO can specifically increase expression of canonical target mRNA and full-length protein in tissues with endogenous gene expression. As these events are naturally occurring, TANGO can upregulate the wild-type alleles in the context of autosomal dominant haploinsufficiency, thus providing a potentially unique opportunity to treat diseases.

### Example 2: Experimental Administration of Antisense Oligomer ASO-22 to Mice

Mice were administered a single intracerebroventricular (ICV) injection of ASO-22 or vehicle (PBS) at postnatal day P2 or P14. Mice were monitored for survival out to 14 weeks. To monitor for seizures, mice are implanted with an 8201-EEG Headmount (Pinnacle Technology, Inc., Lawrence, KS) and seizure activity was continuously monitored from P22-P46. ASO-22 Measurement: ASO-22 levels were measured by Liquid Chromatography Mass Spectrometry (LCMS) SCN1A gene expression measurement Productive Scn1a mRNA transcripts were measured using TaqMan® qPCR assays. Na_{V}1.1 Protein measurement: Na_{V}1.1 protein was measured using a Mesoscale Discovery electrochemiluminescence (MSD-ECL) assay which utilizes Na_{V}1.1 expression in wild-type mouse brain tissue lysates as a standard.

### Example 3: Monitoring Survival of Mice after ASO-22 Administration at Post-Natal Day 2

Mice groups were monitored for survival after ASO-22 was administered. FIG. 2 shows an exemplary survival curve demonstrating 100% long term survival benefit provided by a SCN1A-targeting ASO (termed as “ASO-22”) in Dravet mouse model. WT and heterozygous Dravet mice (+/−), F1 offspring from 129S-Scn1a^{tm1Kea}×C57BL/6J crosses, received a single dose ICV injection of 20 μg PBS or ASO-22 blindly on postnatal day 2, and their survival was monitored until 14 weeks. As depicted, Dravet mice receiving PBS treatment started to die from about postnatal day 20, whereas all mice of other three groups, including Dravet mice receiving ASO-22 treatment group, survived at least 70 days. Kaplan-Meier curve showing DS and WT littermate mice monitored to 14 weeks for survival. Administration of ASO-22 significantly (p<0.0001; ****=p<0.0001 on FIG. 2) improved survival in the Dravet mouse model. 33/34 survived up to 14 weeks, compared with 14/62 animals in PBS-treated group. ASO-22 administration at P2 was able to prevent SUDEP (Sudden Unexpected Death in Epilepsy) in the DS mouse model.

### Example 4: Monitoring of Seizures in Mice

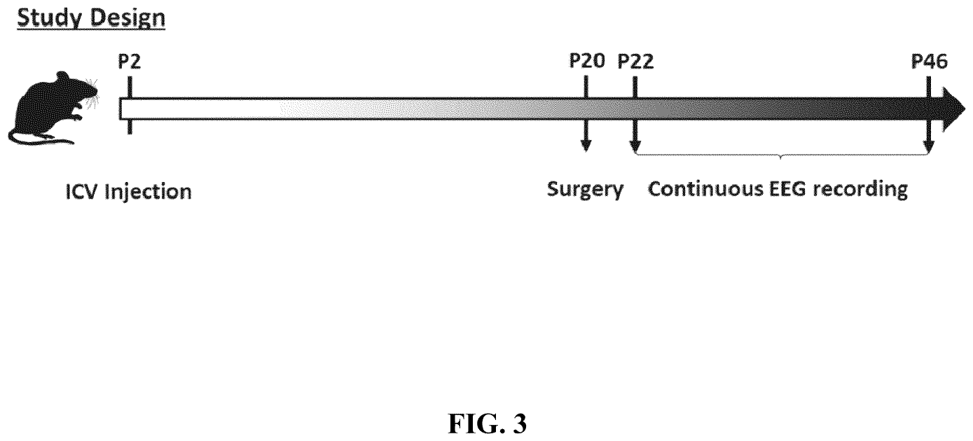

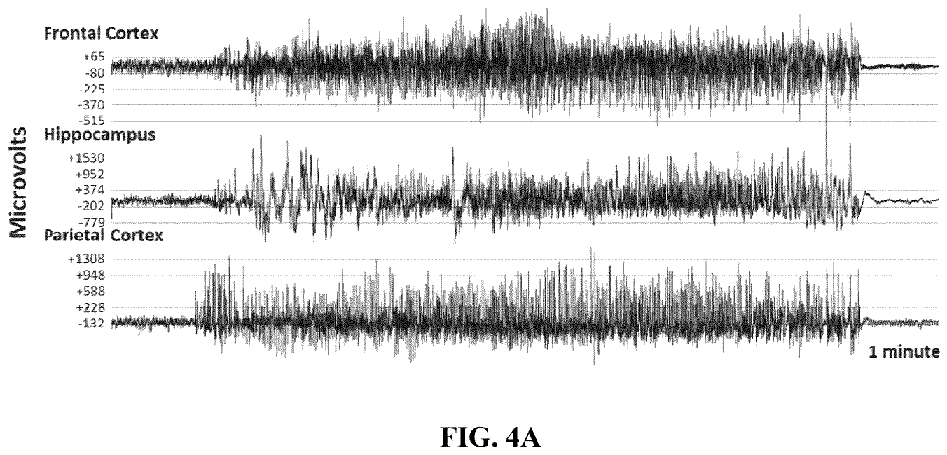

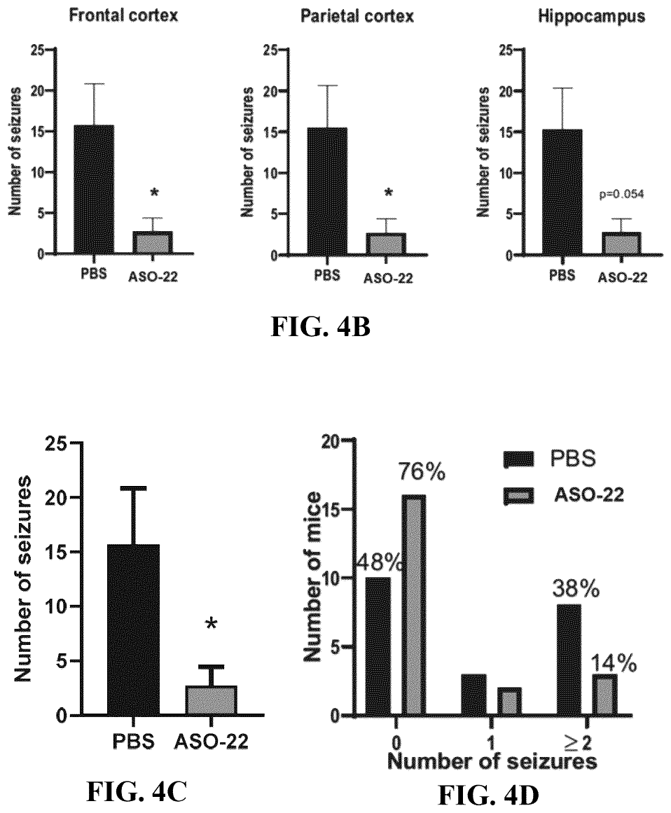

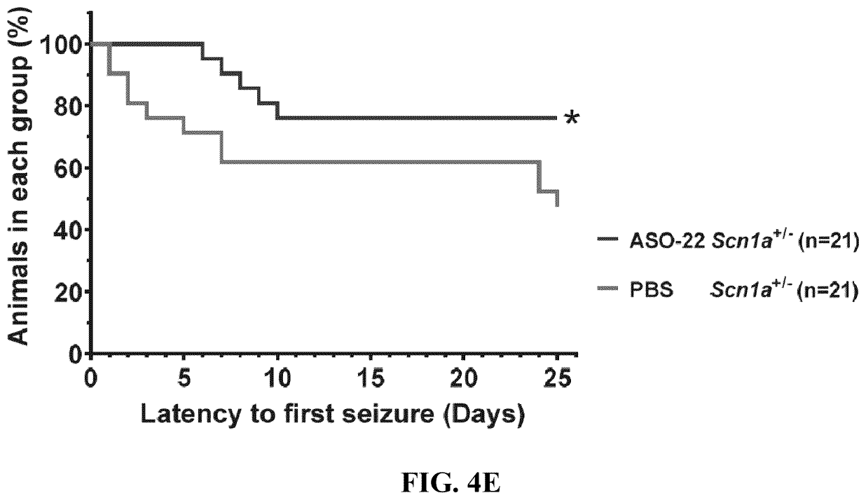

EEG recording was performed on the mice to monitor seizures. FIG. 3 shows experimental design for the EEG seizure monitoring study in DS mice (n=21/group) and their WT littermates (n=11-12/group). Mice received a 20 μg ICV injection of ASO-22 or PBS vehicle at P2, underwent surgery for EEG head mounts at P20, and were continuously monitored by EEG from P22 to P46. WT mice injected with ASO-22 had normal EEGs throughout the monitoring period (data not shown). FIG. 4A shows representative traces from three channels during a seizure event recorded in different parts of the brain of a PBS-injected DS mouse, including the frontal cortex, the hippocampus, and the Parietal cortex. As shown in FIG. 4B, the number of recorded spontaneous seizures in the mice were shown to be reduced significantly in DS mice demonstrating a robust seizure freedom. In all mice, seizures were predominantly generalized as observed by the similar data obtained in the different areas of the brain. FIG. 4C is a summary of effect of ASO-22 on total number of spontaneous seizures (generalized and focal) recorded between P22 and P46 in DS mice dosed with PBS (n=21) or ASO-22 (n=21). *Indicates p<0.05. Additionally, as shown in FIG. 4D, the number of seizure-free mice increased after ASO-22 administration at P2, while the number of mice having 2 or more seizures decreased compared to control. Wild-type mice did not have any seizure regardless of treatment. A 50% increase (76% vs 48%) in the number of mice that experienced no seizures following administration of ASO-22 at postnatal day 2 and monitored by EEG between postnatal day 22 and day 46. Reductions in the number of mice that experienced two or more seizures following administration of ASO-22 at postnatal day 2 compared to placebo (14% vs 38%). An 80% reduction (3 vs 15) in the average number of spontaneous seizures detected between postnatal day 22 and day 46 after treatment with ASO-22 compared to placebo (p<0.05). ASO-22 administration at P14 was able to significantly reduces SUDEP in the DS mouse model. Furthermore, administration of ASO-22 significantly prolonged latency to first seizure in DS mice during the recording period (p<0.05, FIG. 4E). There were non-statistically significant trends suggesting increased numbers of seizure-free DS mice (48% PBS vs 76% ASO-22) and decreased numbers of DS mice having 2 or more seizures (38% PBS vs 14% ASO-22) over the assessment period following ASO-22 administration.

### Example 5: Detection of ASO-22 Exposure and Protein Expression Levels in Mice Brains

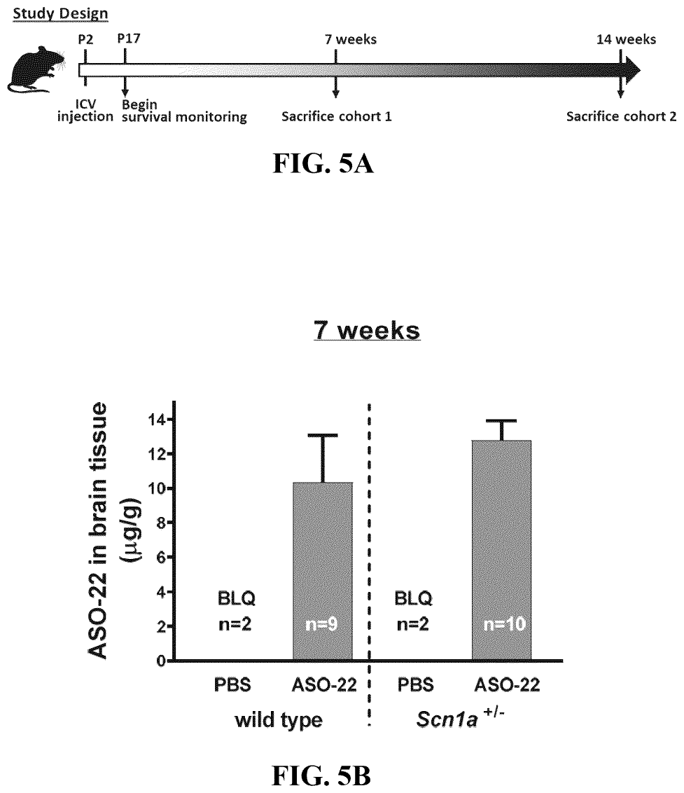

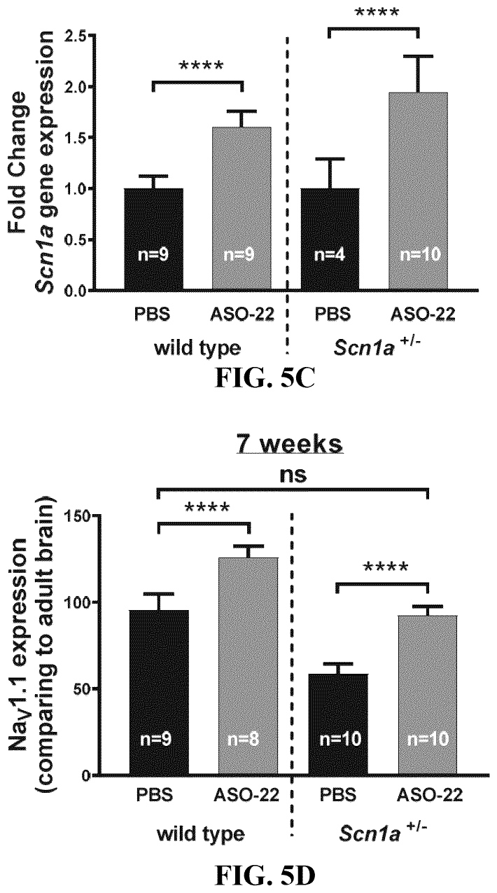

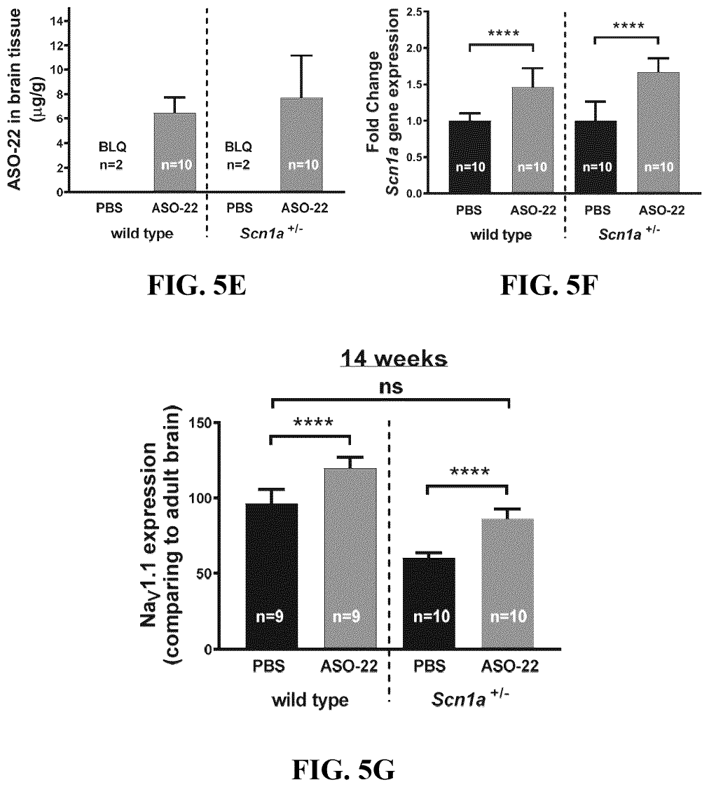

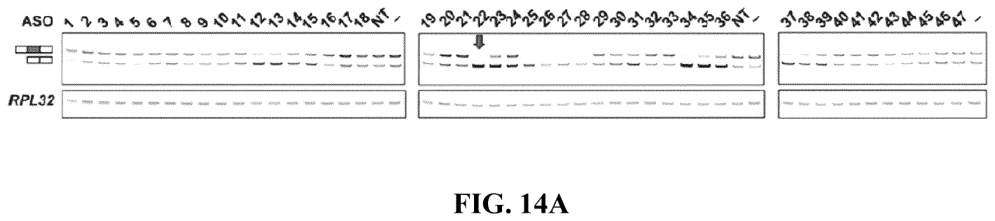

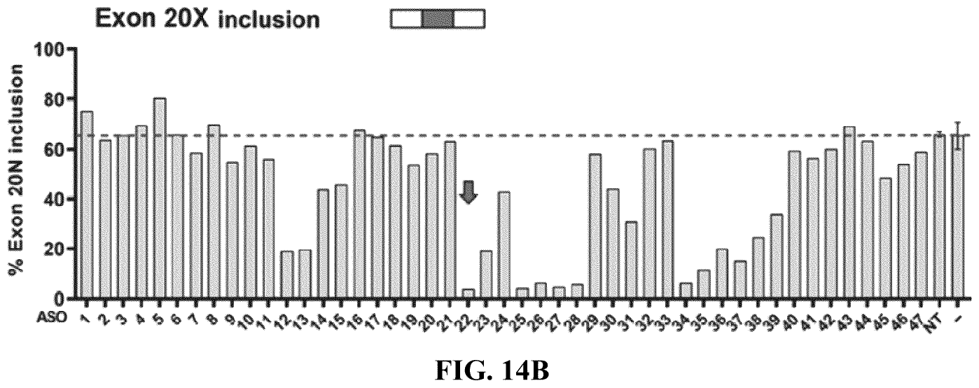

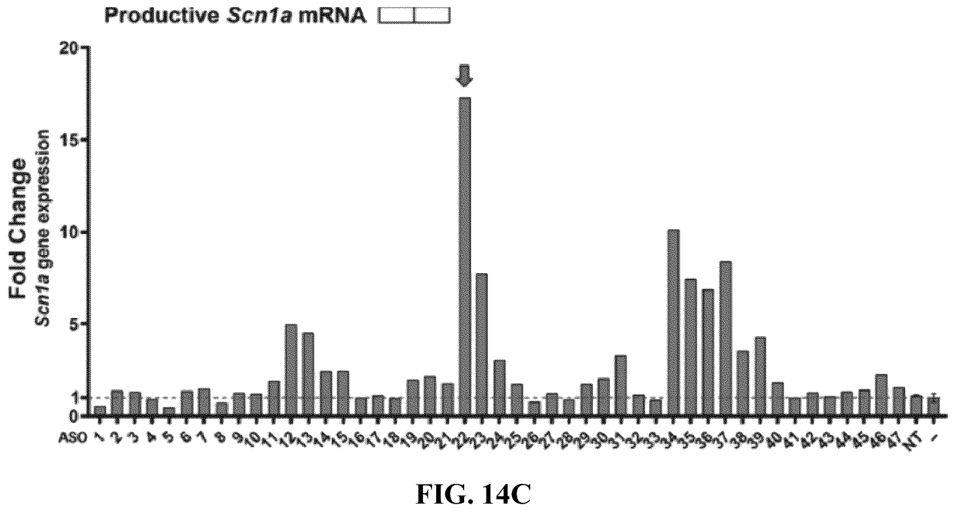

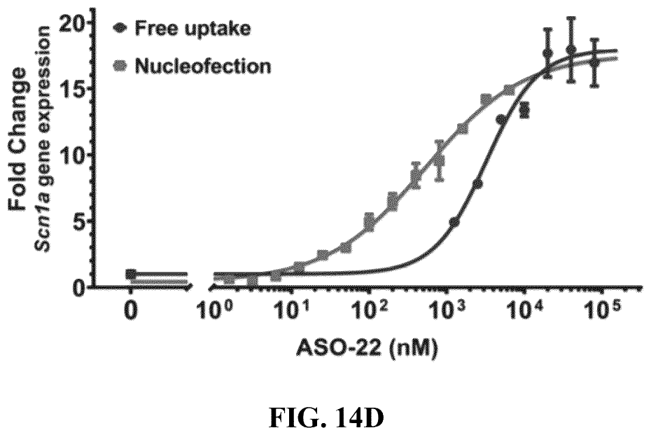

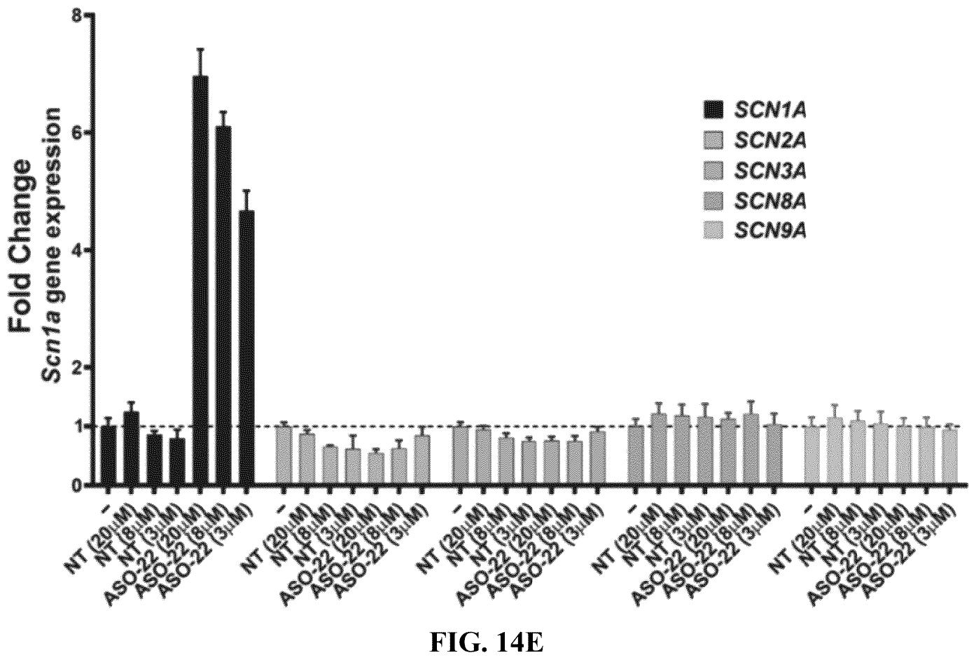

Mice brains were collected in order to detect exposure of the ASO to the brain tissue as well as detect the protein expression levels in the brain tissue. FIG. 5A shows the experiment timeline. Wild-type (WT) or heterozygous Dravet mice (HET) F1 mice from 129S-Scn1a^{tm1Kea}×C57BL/6J crosses were subjected to an intracerebroventricular (ICV) injection at postnatal day 2 of 20 μg of ASO-22 or PBS. The brains of the mice were collected at 7 weeks from a subset of mice in all mice groups. A second collection of brains was performed at 14 weeks from another subset of mice in all mice groups. The brain tissues were shown to have a robust and long-lasting exposure to ASO-22 (˜13 μg/g at 7 weeks; FIG. 5B and ˜8 μg/g at 14 weeks; FIG. 5E) as measured using Liquid Chromatography-Mass Spectrometry (LC-MS). The expression levels of Scn1a productive transcript were increased after ASO-22 administration by ˜1.5-2 fold in both WT and DS mice at 7 weeks (FIG. 5C) and 14 weeks (FIG. 5F). FIG. 5D (7 weeks) and FIG. 5G (14 weeks) shows an increase in Na_{V}1.1 protein in ASO-22 treated DS mice. Na_{V}1.1 protein increased in ASO-22 treated DS mouse brains to levels indistinguishable from PBS-treated WT brains at 7 weeks and 14 weeks. Na_{V}1.1 protein in the brains of ASO-22 injected WT animals also increased over PBS treated WT mice with no obvious adverse effects.

### Example 6: Monitoring Survival of Mice after ASO-22 Administration at Post-Natal Day 2

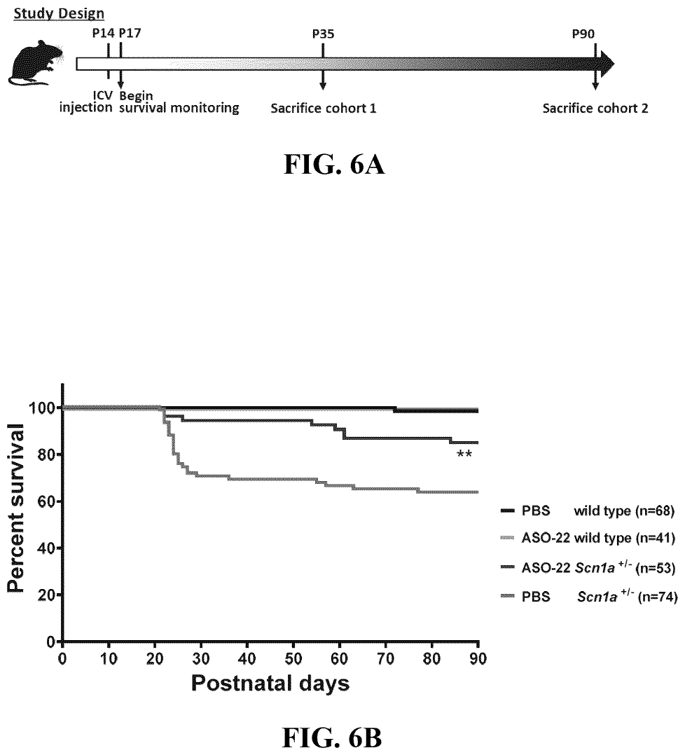

Mice groups were monitored for survival after ASO-22 was administered on P14. FIG. 6A shows a study design timeline with the mice injected on P14 and monitored until P90. FIG. 6B shows an exemplary survival curve demonstrating long term survival benefit provided by a SCN1A targeting ASO in Dravet mouse model. WT and heterozygous Dravet mice (+/−), F1 offspring from 129S-Scn1a^{tm1Kea}×C57BL/6J crosses, received a single dose ICV injection of 60 μg PBS or ASO blindly on postnatal day 14 (which was around the time of disease onset), and their survival was monitored until P90. As depicted, Dravet mice receiving PBS treatment started to die from about postnatal day 20 resulting in a lower survival rate at P90 than the DS mouse treated with ASO-22. 47/74 of the PBS treated DS mice survived up to P90 whereas 45/53 (p<0.005) of the DS mice treated with ASO-22 survived up to P90.

### Example 7: Detection of ASO-22 Exposure and Protein Expression Levels in Mice Brains

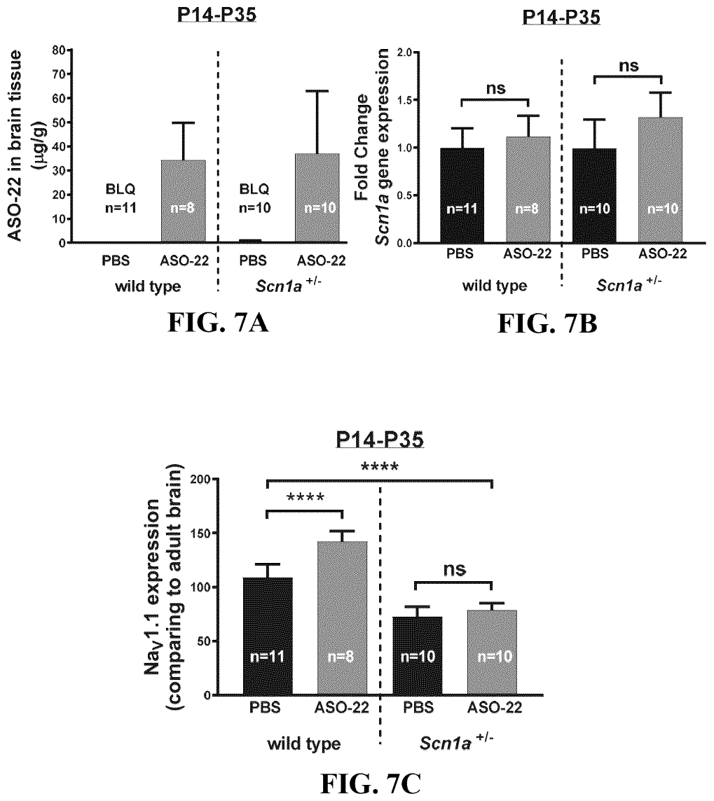

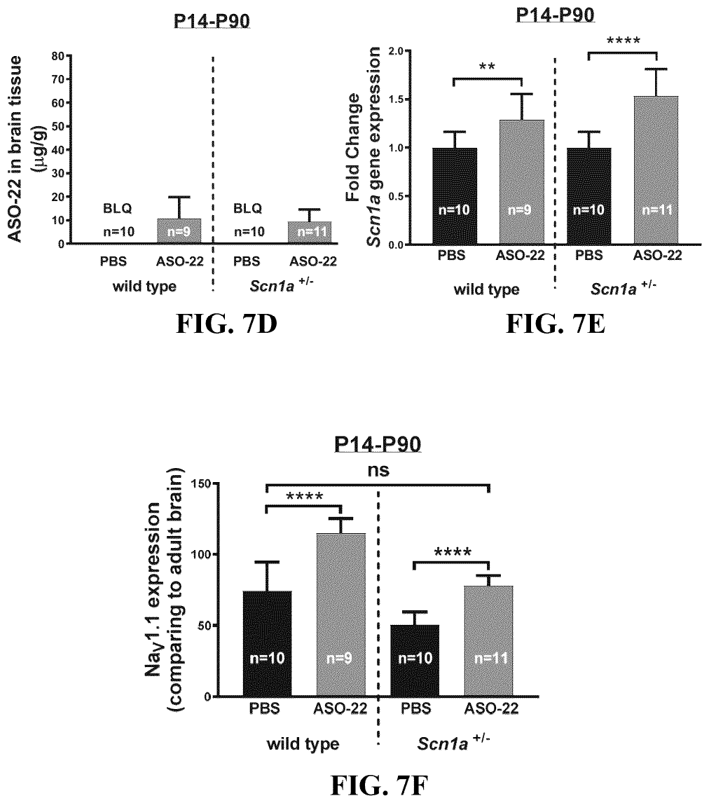

Mice brains were collected in order to detect exposure of the ASO to the brain tissue as well as detect the protein expression levels in the brain tissue. FIG. 7A shows the experiment timeline. Wild-type (WT) or heterozygous Dravet mice (HET) F1 mice from 129S-Scn1a^{tm1Kea}×C57BL/6J crosses were subjected to an intracerebroventricular (ICV) injection at postnatal day 14 of 60 μg of ASO-22 or PBS. The brains of the mice were collected at P35 from a subset of mice in all mice groups. A second collection of brains was performed at P90 from another subset of mice in all mice groups. The brain tissues were shown to have a robust and long-lasting exposure to ASO-22: ˜ 36 μg/g at P35 (FIG. 7A) and ˜10 μg/g at P90 (FIG. 7D) as measured using LCMS. The expression levels of Scn1a productive transcript were increased after ASO-22 administration by ˜1.5-fold in both WT and DS mice at P90 (FIG. 7E) with no significant change at P35 (FIG. 7B). FIG. 7F shows an increase in Na_{V}1.1 proteins in ASO-22 treated DS mice at P90 with no significant change at P35 (FIG. 7C). Na_{V}1.1 protein increased in ASO-22 treated DS mouse brains to levels indistinguishable from PBS-treated WT brains at P90. Na_{V}1.1 protein in the brains of ASO-22 injected WT animals also increased over PBS treated WT mice with no obvious adverse effects.

### Example 8: Administration of ASOs to Cynomolgus Monkeys

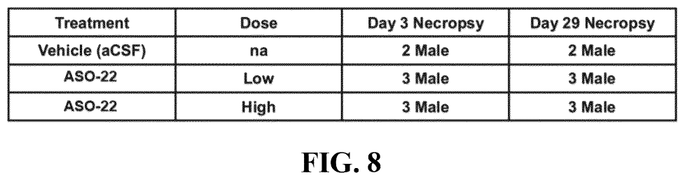

2- to 3-year-old naïve cynomolgus monkeys were administered a single bolus intrathecal lumbar (IT-L) injection of artificial cerebrospinal fluid (aCSF) or one or two dose levels of ASO-22 antisense oligomer (ASO) on the first study day. Two dose levels of an optimized ASO that was active in targeting a nonproductive alternatively splicing event in Scn1a in rodent were evaluated in cynomolgus monkeys for safety, brain biodistribution, target engagement and pharmacodynamics. After a single intrathecal lumbar bolus injection (IT-L) of the ASO, animals were sacrificed and ASO, Scn1a mRNA and Na_{V}1.1 protein levels were measured on Day 3 (48 hours post dose) and on Day 29 (28 days post dose). FIG. 8 shows the treatment, dosing and number of animals used for an exemplary study.

After dosing, the animals underwent a standard clinical and neurological observation and blood samples were collected. CSF and brains were collected at necropsy. Separate punches of various regions were collected for ASO-22 concentration levels, gene expression and protein expression measurements. ASO-22 levels were measured by liquid chromatography mass spectrometry (LCMS) in tissues and by hybridization enzyme-linked immunosorbent assay (HELISA) in plasma and CSF. Productive and non-productive (NMD-inducing) Scn1a mRNA transcripts were measured using TaqMan® qPCR in order perform SCN1A gene expression measurements. Na_{V}1.1 protein was measured using a Mesoscale Discovery Electrochemiluminescence (MSD-ECL) assay which utilizes a recombinant fragment of Na_{V}1.1 as a standard. The ASO was well tolerated at both dose levels with no changes on physical and neurological exams, no changes in food intake, body weight, hepatic function or platelet counts, no complement system activation, and no abnormal histopathology was observed in all tissues examined.

### Example 9: Detection of ASO-22 ASO in the Brain Tissues of Cynomolgus Monkeys

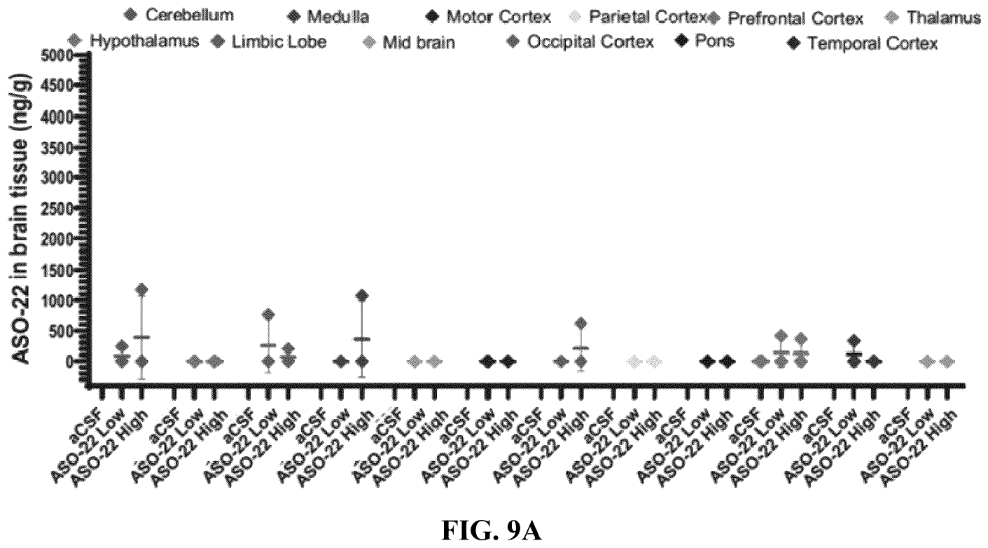

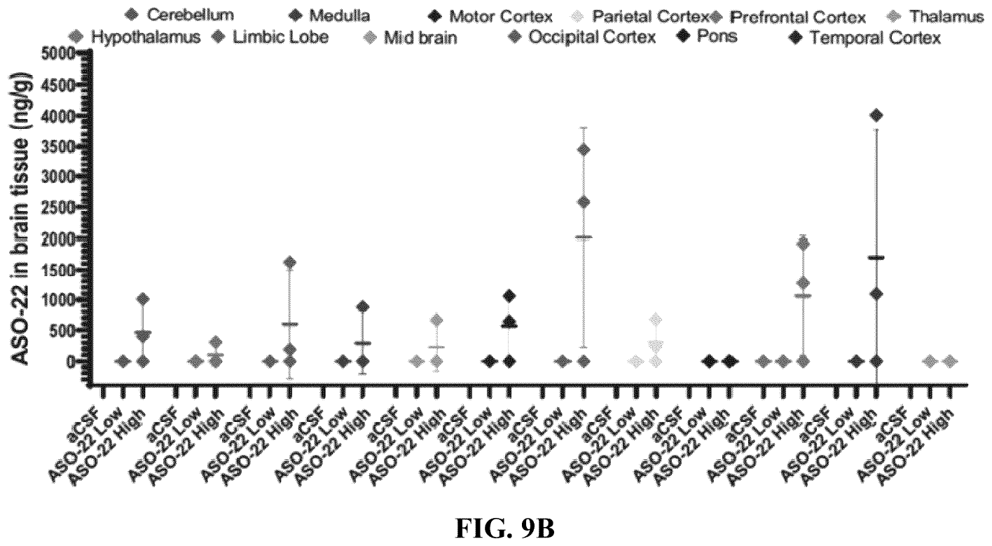

The brains of 2- to 3-year-old naïve cynomolgus monkeys were analyzed for levels of the ASO-22 ASO using LCMS. Cynomolgus monkeys were sacrificed at Study Day 3 and Study Day 29 to observe the levels of ASO-22 in various brain tissues for the various doses. FIG. 9A shows the levels of ASO-22 in various area of the brains of the monkeys on Study Day 3. At the low dose, ASO-22 exposure was below the limit of quantitation (BLQ) in most of the brain regions. At the high dose, the ASO-22 was detectable, primarily detected in the cortex and the cerebellum. FIG. 9B shows the levels of the ASO-22 at Study Day 29. At the low dose, the brain tissue exposure was BLQ in most regions of the brain. For the high dose level, exposure in the cortical brain regions was generally higher than in the deeper structures and the level of exposure was generally increased from Day 3. ASO-22 is observed to distribute broadly in the non-human primate brain at the higher dose level.

### Example 10: Detection of Na_{V}1.1 Expression in the Brain Tissues of Cynomolgus Monkeys

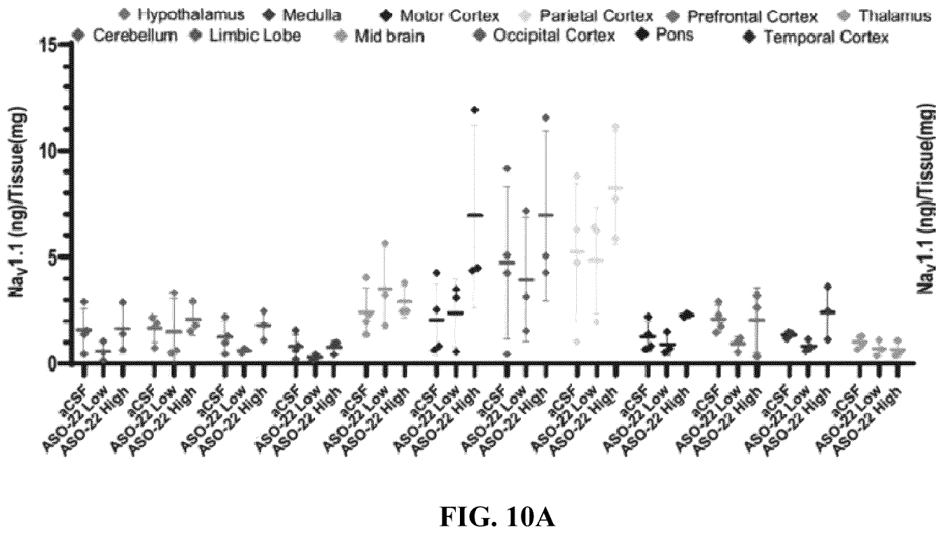

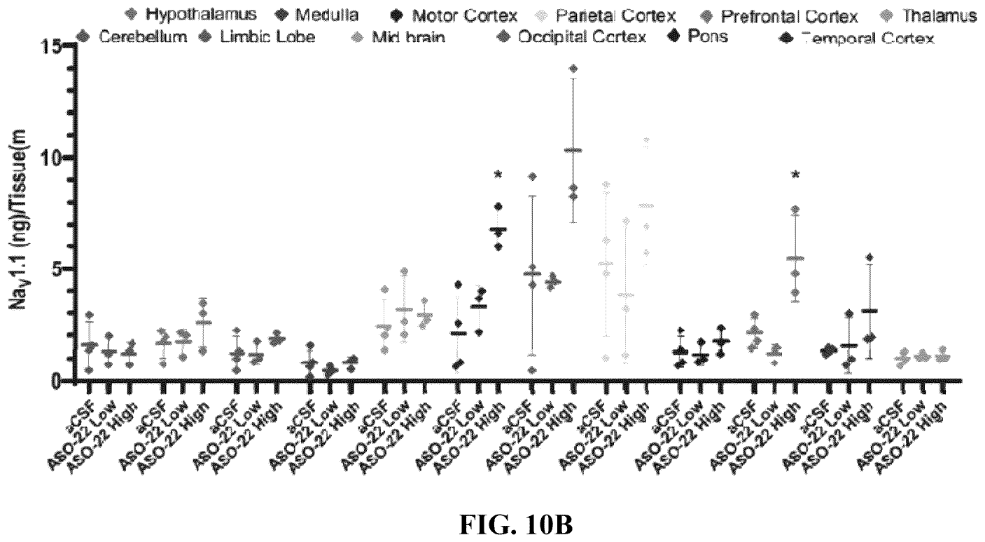

The brains of 2-3-year-old naïve cynomolgus monkeys were analyzed for levels of the Na_{V}1.1 expression. Cynomolgus monkeys were sacrificed at Study Day 3 and Study Day 29 to observe the expression levels of Na_{V}1.1 in various brain tissues for the various doses. FIG. 10A shows the levels of ASO-22 in various area of the brains of the monkeys on Study Day 3. All brain regions assessed had measurable levels of Na_{V}1.1, with midbrain, motor cortex, occipital cortex and motor cortex being the highest. No or marginal change in Na_{V}1.1 levels in brain tissues were observed at the low or high dose levels of ASO-22. FIG. 10B shows the levels of the ASO-22 at Study Day 29. No or marginal change in Na_{V}1.1 level in brain tissues were observed at the low dose of ASO-22. At the high dose of ASO-22, Na_{V}1.1 protein level increased by 1.2- to 3-fold in motor cortex, occipital cortex, parietal cortex, and prefrontal cortex compared to aCSF treated animals.

### Example 11: Detection of SCN1A Expression in the Brain Tissues of Cynomolgus Monkeys

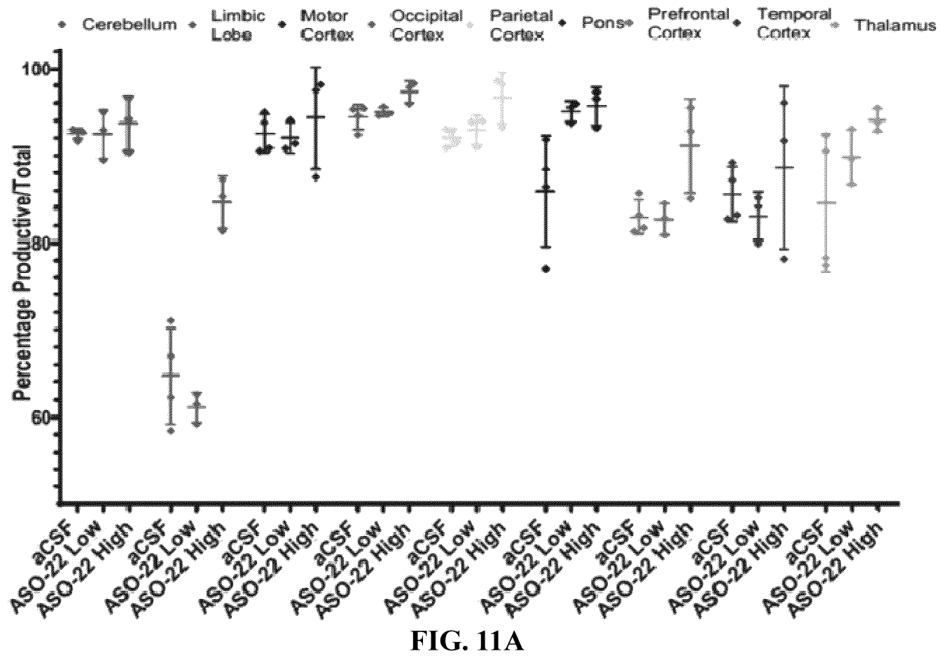

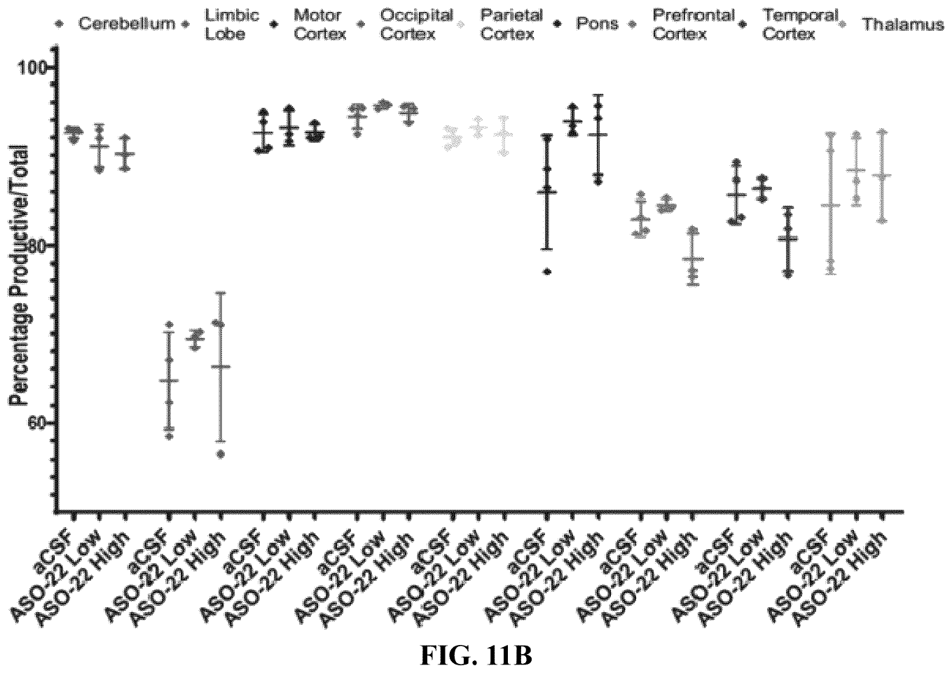

The brains of 2- to 3-year-old naïve cynomolgus monkeys were analyzed for levels of the SCN1A expression. Levels of productive SCN1A gene and levels of total SCN1A gene expression were determined in order to evaluate the target engagement of the ASO-22 ASO. Cynomolgus monkeys were sacrificed at Study Day 3 and Study Day 29 to observe the expression levels of SCN1A in various brain tissues for the various doses. FIG. 11A shows the percentage of productive SCN1A gene expression/total SCN1A gene expression in various area of the brains of the monkeys on Study Day 3. No significant changes in percentage of productive gene expression were observed for either dosage compared to the control aCSF expression at Day 3 for any of the brain regions, indicating that limited target engagement had occurred. FIG. 11B shows the percentage of productive SCN1A gene expression/total SCN1A gene expression in various area of the brains of the monkeys on Study Day 29. Significant target engagement as determined by measurement of productive SCN1A gene expression/total SCN1A gene expression was observed in prefrontal cortex, parietal cortex, occipital cortex and limbic lobe on Day 29 at the high dose of ASO-22.

### Example 12: Detection of ASO-22 ASO in the Plasma and CSF of Cynomolgus Monkeys

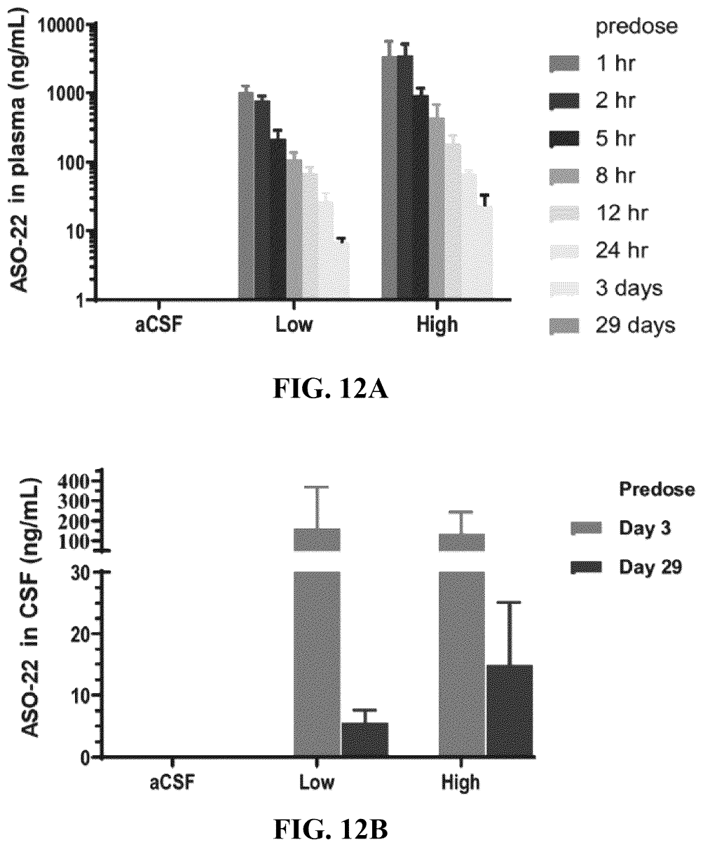

The plasma and CSF of 2- to 3-year-old naïve cynomolgus monkeys were analyzed for levels of ASO-22 after the intrathecal administration of ASO-22. FIG. 12A shows the plasma pharmacokinetics of the ASO-22 in the monkeys for low and high doses. Plasma levels were observed at timepoints from 1 hour from the administration up until 29 days from administration. ASO-22 reached mean peak plasma levels approximately 1 hour (first time of collection) for the low dose and 2 hours for the high dose. The concentrations declined in a biphasic manner. Peak and total exposures of each compound increased with increasing dose. Measured levels of ASO-22 in pre-dose and aCSF-dosed samples were below the limit of quantification of each assay.

FIG. 12B shows the levels of ASO-22 in the cynomolgus monkey CSF on day 3 and day 29. At Day 3, the observed CSF exposure in the low and high dose groups were similar. CSF ASO-22 levels decreased markedly from Day 3 to Day 29 for both doses suggesting a transition from distribution to clearance phase during this period. Mean exposure levels were variable, but slightly higher in the high compared to the low dose group at Day 29.

### Example 13: Identification of NMD-Inducing Exon Inclusion Events in SCN1A Transcripts by RNAseq Using Next-Generation Sequencing (NGS)

Whole transcriptome shotgun sequencing was carried out using next-generation sequencing to reveal a snapshot of transcripts produced by the SCN1A gene to identify NIE inclusion events. For this purpose, polyA+ RNA from nuclear and cytoplasmic fractions of HCN (human cortical neurons) was isolated and cDNA libraries constructed using Illumina's TruSeq Stranded mRNA library Prep Kit. The libraries were pair-end sequenced resulting in 100-nucleotide reads that were mapped to the human genome (February 2009, GRCh37/hg19 assembly). Briefly, mapped reads visualized using the UCSC genome browser (operated by the UCSC Genome Informatics Group (Center for Biomolecular Science & Engineering, University of California, Santa Cruz, 1156 High Street, Santa Cruz, CA 95064) and described by, e.g., Rosenbloom, et al., 2015, “The UCSC Genome Browser database: 2015 update,” Nucleic Acids Research 43, Database Issue, doi: 10.1093/nar/gku1177) and the coverage and number of reads can be inferred by the peak signals. The height of the peaks indicates the level of expression given by the density of the reads in a particular region. The upper panel shows a graphic representation of the SCN1A gene to scale. The conservation level across 100 vertebrate species is shown as peaks. The highest peaks correspond to exons (black boxes), while no peaks are observed for the majority of the introns (lines with arrow heads). Peaks of conservation were identified in intron 20 (NM_006920), shown in the middle panel. Inspection of the conserved sequences identified an exon-like sequence of 64 bp (bottom panel, sequence highlighted in grey) flanked by 3′ and 5′ splice sites (underlined sequence). Inclusion of this exon leads to a frameshift and the introduction of a premature termination codon in exon 21 rendering the transcript a target of NMD.

Exemplary SCN1A gene, pre-mRNA, exon, and intron sequences are summarized in Table 1. The sequence for each exon or intron is summarized in Table 2.

TABLE 1
List of target SCN1A gene and pre-mRNA sequences.

| Species | SEQ ID NO: | Sequence Type |
|---|---|---|
| Human | SEQ ID NO: 1 | SCN1A gene (NC_000002.12) |
| | SEQ ID NO: 2 | SCN1A pre-mRNA (encoding e.g., |
| | | Scn1a mRNA NM_006920.5) |
| | SEQ ID NO: 3 | Exon 20 gene |
| | SEQ ID NO: 4 | Intron 20 gene |
| | SEQ ID NO: 5 | Exon 21 gene |
| | SEQ ID NO: 6 | Exon 20x gene |
| | SEQ ID NO: 7 | Exon 20 pre-mRNA |
| | SEQ ID NO: 8 | Intron 20 pre-mRNA |
| | SEQ ID NO: 9 | Exon 21 pre-mRNA |
| | SEQ ID NO: 10 | Exon 20x pre-mRNA |
| Mouse | SEQ ID NO: 11 | SCN1A gene (NC_000068.7) |
| | SEQ ID NO: 12 | SCN1A pre-mRNA (encoding e.g., |
| | | Scn1a mRNA NM_001313997.1) |
| | SEQ ID NO: 13 | Exon 21 gene |
| | SEQ ID NO: 14 | Intron 21 gene |
| | SEQ ID NO: 15 | Exon 22 gene |
| | SEQ ID NO: 16 | Exon 21x gene |
| | SEQ ID NO: 17 | Exon 21 pre-mRNA |
| | SEQ ID NO: 18 | Intron 21 pre-mRNA |
| | SEQ ID NO: 19 | Exon 22 pre-mRNA |
| | SEQ ID NO: 20 | Exon 21x pre-mRNA |

TABLE 2
Sequences of target exon or intron in SCN1A pre-mRNA transcripts.

| SEQ ID | | |
|---|---|---|
| NO: | Sequence Type | Sequence |
| SEQ ID | Exon 20 pre- | GUUUCAUUGGUCAGUUUAACAGCAAAUGCCUUGGGUUACUCAGAACUUGGAGCCAUCA |
| NO: 7 | mRNA | AAUCUCUCAGGACACUAAGAGCUCUGAGACCUCUAAGAGCCUUAUCUCGAUUUGAAGG |
| | | GAUGAGG |
| SEQ ID | Intron 20 pre- | guaagaaaaaugaaagaaccugaaguauuguauauagccaaaauuaaacuaaauuaaa |
| NO: 8 | mRNA | uuuagaaaaaggaaaaucuaugcaugcaaaaggaauggcaaauucuugcaaaauugcu |
| | | acuuuauuguuuuaucuguugcauauuuacuucuaggugauaugcaagagaaauaggc |
| | | cucucuugaaaugauauaauaucauuuaucugcugugcuuauuuaaaugacuuuauuu |
| | | uccuaauccaucuugggaguuuccuuacaaaucuauauacaaaaaaaagcugaugcau |
| | | uauuaaaguacuauguguaaugauauaaugguaaucuaaaguaaauucuauaucacgg |
| | | uacuuauucuuugugaugauauacuguacuuaacgaguuuuccugaaaauaaugugaa |
| | | ucacacaugugccuaaguaugaguguuaagaaaaaaaugaaaggaguuguuaaaacuu |
| | | uugucuguauaaugccaaaguuugcauuauuugaauauauucaagauuagaugguuag |
| | | auauuaaguguugacugaauuuauaaaacuaguaauacuaacuuaaagauuacauaca |
| | | aauccacaucauuuuuauaacaauaaaguaaaacacuuauaaugaacagaaaauauag |
| | | uuuugacucauuacuauagguaauuuauacauuaaccuuaacuugcaucuuauugguc |
| | | agagucacacaaaauguuauuuuauccuuuucaaagaugcaauaaucauuuuccauca |
| | | ugcauaacagauuagaaauuuugccauuauugacuuauuuuccaugccuuuuuuuacg |
| | | gcaugaagcauuaguuuauagauauauaauauaaaaaauuaguucugcuuuuuuuuaa |
| | | aaaaaaauauuaucaaaacaaaacacugaauugugugauuccaauagaaaaacacugc |
| | | ucuuucaccuccuaagguguaguuacuuuuauggaaacuaagcuguauuuguagacuu |
| | | ccauuugcacuuuguagauuguuuauagccuuauguucucuucucaagucuuauuaua |
| | | aaugucacuuuguaagaacguaggacuugucuucgauuucccuaacauauaugaaaac |
| | | uuuguccucauuaucgacaacucagaacaauauaauacaaguaguccucuuuuauuuc |
| | | ucacagagagccucaaauuuucaccaaaauguuaacagaaauuaucucugggguguau |
| | | aagaauuaagucuguuuuccaauuaaaugucacuuuguuuuguuucagacuggcaguu |
| | | ucaguucuggagaaaaaaaaugucauuuguguacauucuacuugaaaacauguugccu |
| | | gaaucaaaauaauauauuuuauauggcuugugaaaucugaacaaugcuaaacauuuga |
| | | aaauauuauaaaccuuuuacauuugaccauuugaaaguuuauuaaguucauuggucaa |
| | | gugcucagauauuuccauacauuacacuucauuucuauaaaaaagcugaucuuaucgg |
| | | uauacuuuuaauuuucucagaaauaaccauaucuauaauuauuaaucaauaaugccuu |
| | | uuauauuaaaagagguuaguuuuugaaacuuggaguuuuagacauaaaauccuuauaa |
| | | augcugauagugauauaacuaauaguuuaaauggucagauuuaugaauauggcucuau |
| | | uccucauaaugacaacauacacacagcacuaaaaugacuaaucucuucaauacguguu |
| | | uggcauuguagagucaaaauaacguuauaauugauucuauuuuuuauacuucuagugu |
| | | uuggauauuuuauuuuguaaaaauauaaucaugaaugauggugagguuggauauaaga |
| | | augaugauuaugauugggaagugagauuugaacaugcucagaaacucucauuuaauuc |
| | | uuugcccuagcagcauaaaaucacaauagcugcgucaaagcguaacucaggcacucau |
| | | uuuauuuuuguuguucuguuauuuuuucaaagcaugugcuuuuaugcaacauuacuga |
| | | auaaagcauguuguacagugcuugauaagaaguuagaaaguaacaaauaaauuaucau |
| | | cacguugcacuuuguguuuugcauguuuuaugcacauuucuggcugacagcuuuuaaa |
| | | cauuuauuguauuucaaauuuccaguccaagauuuuucaacuuguaaaauuaaacuga |
| | | gugaauugaugucgugaauaucuaggguaaaauaaaauuuguguuuaaauuuguauuu |
| | | uuaauuuccuaaccuaggaaaucuuaaauaccuucuuuuucaaaagaacucaagucuu |
| | | aauggauagggaaacagacggagagcaucaugaacaaaaaguaacaccaaauguucug |
| | | ucauaucagauuucuaacuaauaacaaacuauauauuucuauuuuguauaggauaauc |
| | | uugcuccaacuuggaugggguggagcgcugguuccuccccugagcccuuuauuauggg |
| | | uacuguauuaccccuuuugcuaccuuuaauccuugcacugugacuuauguguagugcg |
| | | gugagcgagcggauugggaagcguacuauuauugcaccacaguagggaaaauacauua |
| | | uuuacauccuaauccccucuuuucaauugucuuaaauuucauuugaaaaaaaaaaaaa |
| | | ccuuuaugaauuuacccucuguggauuuuaaccccaaugguugauaucuuuauuaagu |
| | | uucauugaauaugauuuaguuauguguauauggaguuauccaucuuuggpggagauua |
| | | cuggauuggugagggcgggggacccugguguagaaugauuaugugaaaaaacaauuua |
| | | acuuguuaagcucaugauacuguuugaggcauacagccccugcuguuuaguacauugg |
| | | ucuggguccugaaaauuaccaguuagauaccaucaguugauuauugauauguaugagc |
| | | agauacuagggugcaauauuucagguuucauaagacugguauugauugugaccacucu |
| | | cauuuuuuauuguguaaguucauaugggguuauuuucaaaauguuaacaaggcaaaaa |
| | | uauauuaagaaauaguugaauaagcacaugugaauuguguuguaaacaaaaaguuaga |
| | | auaaaaaaauccacuuauuugaauuaugcagaauagaauacauaccuagaaauaaaac |
| | | aaaaacgucuuaucaugaguauuaagauaaaauuuaaggcauaaacucacuucuuaga |
| | | auaaguaacucccaacuaacuuucuaggauuuuaaaacauaacacagugaaaacauac |
| | | auaaacauaacucuacauuuuauuuauucuuaaaguuuaaguguauuauacaagaaga |
| | | agaguuuauauucgagagacagaaaaagucagaauuuuuguuuggaucaccaauauau |
| | | cauagcuuacaaaaaaacugucuuaauuaaaacccacaacauaauuuuuuuagauuuu |
| | | uaagaaagauucuauuuauucuucuuuauacuuaaaaauggaugauuccuacuuugcc |
| | | cacuuuuauuuuuauuucacauagauuuucuuuauuucuauuagagaagcacuagaau |
| | | ucaugaauaguguugauuugaaguucaaaguaauuaauucagauaaaaagacauuucu |
| | | gcauguaugaaaauuuucuaaugugaauuugcauauuuaauuaucaauccuucauuua |
| | | guguagacuuauuuuuaaaaaugcagguaaugaaccagaaauagaauugguugugcua |
| | | gaguagagaaacuuuauuugaugauuguuuugaaaaaaaagcuucugagaagaaacaa |
| | | ccucuaguacaguauuaauucauuaagauagcuccuuucucagacauuuccuuucaug |
| | | uagccugaaaguucaauuugaaauuuguucuuucccaauuuauucagacuaauucugc |
| | | cuacuuucuucccccccauaagaaccaauuacugcagcuuuauugagacugaaaaaag |
| | | uuaauacaccuccuucuuugcugaaccaaggaauggcuuggaacucuugcgaaaagac |
| | | aaucuuuucuaugaucuuucauugucuaauuuaauacaucauaaauaugacuauagcu |
| | | uuguauaauaaacuccccaauacugugcccagauguuuucuaagauaaaguuauuuua |
| | | uguucacaaaaaaaauaaaacuuuucucugggccaaauguaugccaacuuugcaaauc |
| | | auauccugaagugcacugcugcagaguacaugcuugcgucauaaauuccauagaguuc |
| | | gcuuuaacucuaaaucaauccccaguuucaaaguaaaccucucaaacauauuaccuaa |
| | | gcacaaacuucuccccugugcucaguuccuuaauuauucucaucccauauucagaaau |
| | | aacauuuaaaaauuaugcuuugaucaauaaauacuaaucuaaacuuugcuucauuaac |
| | | ccauucauuuuuugucaaccauuauuuuauuccuauauucaaagcucucugguauguu |
| | | cuuauauucaagacacucaaggccccuggaagauucacgaacauauguuugcaucuua |
| | | aauuuuuagaaaaucuuacaaucugucaggauuacacugaacucuaguacagaguaau |
| | | auggguaccagauaagugggagcaacucuuccacguagacuggaaacagcacuaaaug |
| | | cuauuuauaggcuacuuucugaacuuaacuuguuuuaaccucauuuuucucauaugcc |
| | | aaaugagaacgcaauacugaauuaucuguacaguucuguucaguacuagaauucugau |
| | | ucuugaauucaaaggggaaaacauuccucuuuauuuuggaggcuaaacugggggacaa |
| | | aguuaggcuccaugaaagaagugcuauuugaacuaaagccuuuaagaggggagaguau |
| | | uucagaagaggagcuauuagacaaggaauuucaauguaaauggcaucucaaucaccug |
| | | gcaauuauauuagcacacgguuauuauauuaauugaaguggcaugaaguauagaugac |
| | | cagggaaguuaaaacuggaaauauagauuguggagugaugugaauaccaagguaagaa |
| | | aaauauuuguuaguuaccagagagccaauaaauaacuuucaagugggacuuggggaag |
| | | auuaguucaucuuacauagauuaaaugaaggagaagguuaggagacagaugacagugc |
| | | aaguaugaaauaacagagcgcaguucuagguggugacugugagaauggaaaagaggug |
| | | gcaaagcugagaaacguuucaaagaaaaaaugugagacagguaaugugaaaagaaaau |
| | | cgagaaauagguauagauaaucaguguucugcucauacucuaaauuggguguugaagg |
| | | caaaauacguauuuuaauuaguacucuguguauacacacuagaaacagcauuguaauc |
| | | uggauaguggacaaaauauucagaaaagaagggaaauaguaacuugauuucaauuucc |
| | | aaaucucuaaucugaaagaaaucuaauucuauucauccauuuaaaauaaauuauauaa |
| | | cgagaauuuaugaaguccauuguauuaaugcagacagucagaugagauaaggcaaagu |
| | | gucacgugucagcuugguaguugcaucggccacaucauuugguucugccuggauaacu |
| | | caaccaaauuaauuuuucauacucauccccuccaccuuugucauuacugguauucuua |
| | | uuuucuuuggcccacuuaucacacuguuuuauguuccccagaaggccuagaguucuuu |
| | | acaggcuuuaaacagggaucagaaguauaagaaauuggcucauguauuuuuuuuucag |
| | | acaggcaguuaaaaaaaauuguucuaaaaauacacuggcaucaaauggcaaauagaag |
| | | auguuuugacgacuacuuccauuggaucagacugacaagaauaauacaagcacauagg |
| | | uggaauuaaacuuagcuauuaauguccaaguuugaggcagcugccccuuauaagcauu |
| | | uuagggucuguuuuuagcuucccucuuagccacuccugugcagcuccagugggaggua |
| | | uggaggaaaaagcaaggaagccaucccuauguuguuuccaaacaugaacacucaagau |
| | | uuuuaacuagugguccagaaguaaagagggggaaaacauccuucuauagaaaaaaaaa |
| | | aaaguagauaauaauugaacacagaacuucaugugaucacaucagauuugagaacuau |
| | | guauggcaucccucuuuuucuuauuuuccuaagaaaugauuucuauuauguuucauuu |
| | | gaaauaaguuuuuugaauuaaacucaguaaaugaaacaacugacaugacuggagcuug |
| | | aaauaaacgaugugaugaucuaaugaaauacauaaugcaaauugucuugcuucuuaug |
| | | caaaaauuauuagucauagcaaugcaugaauaauuaaagacaauuuauauuagguauu |
| | | uaauaauauuuuuuauauuuaucaucugaauuuuuaaguuauuuuaaaaauauauugg |
| | | ucaaaucaacucagguccaaauguuuuaguuuuguucuuuaauauauugccuuuuuaa |
| | | aaugaguuaaacuucuguauaggcuuuuuaacuuuucuuuauucugauaacacaauuc |
| | | ugacuucaucuggcagcaaguuccucugauuuuccuuuuccuuuaaccuuuuaaugcu |
| | | ucucccucccuuuuuuuuaaaaacauuuuuguuucauuuucuugguuauauugccuau |
| | | aguuguuuuccuaaguguauugcuuaagaaaaaaaaauugaauuuuaagauuuuuuug |
| | | aaccuugcuuuuacauauccuagaauaaauagcauugauagaaaaaaagaauggaaag |
| | | accagagauuacuaggggaauuuuuuuucuuuauuaacagauaagaauucugacuuuu |
| | | cuuuuuuuccauuuguguauuag |
| SEQ ID | Exon 21 pre- | GUGGUUGUGAAUGCCCUUUUAGGAGCAAUUCCAUCCAUCAUGAAUGUGCUUCUGGUUU |
| NO: 9 | mRNA | GUCUUAUAUUCUGGCUAAUUUUCAGCAUCAUGGGCGUAAAUUUGUUUGCUGGCAAAUU |
| | | CUACCACUGUAUUAACACCACAACUGGUGACAGGUUUGACAUCGAAGACGUGAAUAAU |
| | | CAUACUGAUUGCCUAAAACUAAUAGAAAGAAAUGAGACUGCUCGAUGGAAAAAUGUGA |
| | | AAGUAAACUUUGAUAAUGUAGGAUUUGGGUAUCUCUCUUUGCUUCAAGUU |
| SEQ ID | Exon 20x pre- | gauaaucuugcuccaacuuggaugggguggaggugguuccccccugagcccuuuauua |
| NO: 10 | mRNA | ugg |
| SEQ ID | Exon 21 pre- | GUUUCAUUGGUCAGUUUAACAGCAAAUGCCUUGGGUUACUCUGAACUCGGGGCCAUCA |
| NO: 17 | mRNA | AAUCCCUAAGGACACUAAGAGCUCUGAGACCCCUAAGAGCCUUAUCACGAUUUGAAGG |
| | | GAUGAGG |
| SEQ ID | Intron 21 pre- | guaagaaaaaggaaaacucugcagcguuguauauugucaaagcuaggcugaguucaac |
| NO: 18 | mRNA | uuaacuaacgaaaaacacgugcaugcaaaaggaauggcaacccuuugcaaacuugcua |
| | | cuuuacccuuuucucuguugcauauuuacuucuuggugauaugcaagagaaaaucggc |
| | | cucuuugaaaaugauuuaauaucauuuaucugcuuugcuaauuaaaaugaccuuaguu |
| | | cauaaucgaucuugggaguuuccuuauaauuccuaauacaaagggggaggggcagaua |
| | | cucucuuaaagaacuaaguugagucauguaauaauuaccuagagauaauuuuguuuca |
| | | uaucguucuccucuaugacagcccaucaguacuuaagggauccuauggaaaguaaugu |
| | | gaaucacaaauguguaugaauacaaaggaaaaaaugaagaauuguuaaauguuuuugu |
| | | cuuuacaaugccaaaauucucauuauuugaauauauccaagggcagauauuaaccauu |
| | | gacuggaguauaauaauacugccucaacuguaacuaaauuaaugacauugaauaagua |
| | | agacacuaauuuaauuacuauaaauacauacacaucuuaugacaauacagcugauaag |
| | | gaaaaagaacauguauuuuuauucauugccauacauggcucgucaaccuuaacuuaaa |
| | | ccucgguucucaguuacacagaguuuuauguugcucuuuugagcaaagcauuauuccc |
| | | cucuccauaauucaacacguaucagauuuuugcauuuauuggcucauuguuaugauga |
| | | uuauuucaaagcauuauacaaucauuuauagaagaugugccgugugaaaguuauuuuu |
| | | uuauuaagauccaaaauuuacgcucuuuaaaccaaucagaugaaauguauaaggcaaa |
| | | gagugcucauuguccugacacuuacaaaccaaggcuccaacaaacaggcuccucucug |
| | | caccacauagagcgcuuucagccuguguccccccauaaaaaccuauuauaaauuuuau |
| | | uauacuauacuguaagaaaccuguccaauuuuuaauuucucuagcacauaugaaaacu |
| | | ucucuucaguagauccaaguaagcacaaaggagcuuugauucucacacaagaaaucac |
| | | auuuguauuaaaaauguaucauaaauuuucucccaauuaugcaaaacuuaaaugcuuu |
| | | uccaauuaaaagagcacuuucguuucagaauagcaauuucaguugucaaggaaaacau |
| | | uguuuuuauacauuuuauauaaaaauacaugagcuaaauuuaaauucacauuuuucaa |
| | | cuuuuuaugguuuuuuuauguuucuuuuucuuucgcauuuuuuaacaauccagccauu |
| | | accucuccucccugucccccuccuauaguuucucaucucauuccuccucccccuugcc |
| | | ucugagaggaugcucccucccucacuaggccucccucuuccccagpggcuucaaguuc |
| | | cucaaggauuauacacaucuucucccacugagaccaggccaggcaguccucugcuucu |
| | | gcccagccuguguauguuccugcuugguagcucagucucuggaagcucccugpcgguc |
| | | ugcguuaguugggacugcuggucuuccuauggaguuacccuccccuucaacuucuuca |
| | | auccuuccccuaauucaaccacagguauccagacuucaguccaaugguugaguguaaa |
| | | uauuggcaucugucucugucagcuguuguuaggggcucagaggacagccaugcuaggc |
| | | uccugccuacaagcagcacaccauaacaucaguaauagugucaggccuuuaaugaaua |
| | | augcuacguauauaagguuguuagauuauacuucaacuuuugaucuuuuagaauauua |
| | | uuaaauccagucguuuauuuuuuauauguaauauugacuuucccauaacaaaugaguc |
| | | uauuuuccuuuuguguagaaauaacuuuaucaauuauuuguuaauaaugcuuuuguca |
| | | auuauuguugauaugcuucucuuuuuuaaaacuggagucaucaaaacaaaaauucugg |
| | | guagauauuacgaagcugacuccuuggucagcuuggcacauagugagaccacaaauau |
| | | cucaaggucacggcaauuccucaccaccaguuuggcauugugaagucaaaaccaaccu |
| | | ucuguugauucugguuuuuguauuucuaguaugagacauuuucuacuuuguaagagua |
| | | uauaacuguggauggcggcgagguugggcaugaugaugauuguaagccpggaagugag |
| | | cuagaguaucuucagaaacucucacuuuauccuccuuggcagcauagaaccgcaaucg |
| | | cugugucccgagugucaaccaggcagucauuuuuguuuuggguuuuuugucacucuuu |
| | | caaagcaugugcuucuacgcaacacuaccaaacacagcaugcugcauagugcuugaga |
| | | aggaguuagaaaguaacaaacgaguuaucaucacguugcccuuuguguuuugcauguc |
| | | uuaugcacacuuuuggcugacagcuuuugaacauuuauuguauuucaaauuuccaguc |
| | | caaauuuuuuuuucaacuugugaaauugaacggaaugaaccgaugucgugaauaccua |
| | | gggucaaauaaaacuuguauuuaaauucguaguuuuaauuucccaagcugggaaaauc |
| | | guaaaaaccuuuuccaaagaacucaagucuuaguugcuagggaaacaggcagugagca |
| | | ucauauacaaaaaguaacaccaaauguuucugucauaucagcuuucuaacuaauaaua |
| | | aacuauauauuucuauuuuauauaggauaaucuugcuccaacuuggaugcgguggagc |
| | | ggugguuccuccccucagccccuuuauuauggguacuguauuaccccuuuugcuaccu |
| | | uuaauccuugcacugugacuuauguguagugggauugagcgagggagugcgaagcgua |
| | | caauugcaccacaguagggacaauacaggauuuauuuccaaauccacuacuuuuaaug |
| | | agcuuaaacuucuuuugggaaaaaaaaaguuaucucugacuuaccaucuguggauuau |
| | | aaccccagaaguacauaucuuuauuuacguuucacugaauaugauuuagcuauuuaua |
| | | cuucauuguccauuuauggggaaauuacucaauuggugagggugggcgaccccuggug |
| | | uaggaugcugaugaaaacguuuucauuugucaagcucaugguagugacagagcauaua |
| | | guccuuauuuuuucaacacacugcucuggucccucaaugggccagucacauuccauca |
| | | guugaucguugaugugugccgagcagugpcuaaagguacaacaggccagguaucucag |
| | | gguugccaaugguuaugaucauucucaucuuuauugcauaaaaauguguuauuugcag |
| | | aaaguagcaaggcaagaucccugugaaacaagggaauacaaaaaaaaaaaaagaugug |
| | | cuuuaaguuauaaaaccaaaacaugugaaaagucaacuucauugaaguauaaagaaua |
| | | ggauaugcaugaaaaacaaaaaaaucaugagcacuaagaaauugguguauaagcccaa |
| | | cuccuuguaagcuaccccaauuaacuucccagaaucuuagaaggcaucacagugcacc |
| | | ccaaaauaaaaagccaaacugacacuucugcuuccucuuaaaauguaggagucuugga |
| | | uaagaaagauaauuuuuauuuguuuggaagaaaaaaaaauuguuuggauaauugaggc |
| | | auuuaucuaucaaaaauauuuuaucuuaauaaaauuucacaacacugauuuaguuguu |
| | | ggcuuuucuaaaaauuuuuuauauuucauauuaagaacucaugauuuuuacuuuccau |
| | | uuuuuaaauucuuauucacauaugguuuuucucuauuucuuagaaaagcuauagaacc |
| | | caugguuuccggugacuuaaaaaacuaaucuaaaugucuucacuuagaugauacuuuc |
| | | aaaugcacugaaauuucuaauaucaacaagaauauuugccugguccuaauuuuucacu |
| | | gauuuaauaaaaaguaugaacccuaaagaagaaauagacuugaagaacugguugugug |
| | | acaauacagaaauucugccugggagauguccuuuuaaaacauuguuagaagagacaac |
| | | cucuacaauccacccauuaagcauacuucucucuuagacaucuccuuuuauguaccuu |
| | | auaaccucaauguguucuuuccaauugacuuagaccaacacuucccagcgcaucccac |
| | | augggagccaauuacugacucucccuagagacugcaaagaauuaauauuguagaacca |
| | | agggaugguugggcucuugpgagaggcaauccguuugugaucuuuugcucuggauguu |
| | | aaugaaaucgcaacuuuaaguggauuuucaguggcaaauccucugauccuaugccaaa |
| | | uguucucuaagacaaacauccuuguaaaauaaaugucucacugcgccaaaucuauggc |
| | | aaauuugcacguuuuuccugaacugcauuccuauauaguauuugccauccugaauuca |
| | | cuaaugggcauuacuuuuaguucaaaaccagucccuucuucaaaggaaaucucuccca |
| | | uuuauuacauuaugcaaacugcuuucuuaugcagugguuaaauccuuagccaggcaag |
| | | uaugaggacuggaauuuggauauccagaacccucagaaaugucggaugcgcauaguag |
| | | cuuacauguaauuccagagcuagaaagaugaaacuagcccuguccucaagcucugaau |
| | | ucaguuggugcaauaaauaagaaagaauccccccccccgaccccuacaucucuuuccu |
| | | cuacaucuauacauguauuucccauacagcucuaugcuuccacauauaugcucacaua |
| | | ugugcaugcacacuugcacacauauguacacuugacacauugaagcaucauaaucuaa |
| | | caauuggaauauaagaaaauauuuaacuuucacacagagcagucagagaaaaccauaa |
| | | gagguugaaacucuugaaaaaacuugcaggauaaacagaaaagaguaugagaugccaa |
| | | uucuggucuacuuuucugaaccuaacuuguuuuaccuucauuagucuaauuuuccaaa |
| | | ugaaccccaagcaccaaauuguccuuauugcucuuuccaguacuaaauuauaauuccu |
| | | caauucgaaggcaaacacucucaucuuuguuaaggauguguagaguuagacucaauaa |
| | | acgacauguauauuugagcuaaagccuggaggagggagauuauuucaagggcaaguac |
| | | uuggcagggaguuucaaagaaagaaggcucucaauucucagacuaacaccuuagcgug |
| | | gagugacugucacagaggagcgugaaguguaugucaccaguuagcgaagcugaaaggg |
| | | gaaauguaucaggcuugugagaauccuucagcagccaagucuagcaccugagcacaau |
| | | ccccagaacugaccccacaugguggaaaggagaggaauaauuccugcaaauuuuccug |
| | | ugaccuccacccaagugcuauagaaaaugcaugcaugccccacaugcacacacaaaua |
| | | aacauaguugcaaacuguuuugaggaaaauaaccucacaaacugucgagugauguaaa |
| | | ugccaaagaaagagaaauguuuucuaauggcuagagaaccauuaaggaauuuuucaaa |
| | | augggacaugpgauagauaaauuuaguauccauacugaaagaaggcaagcaaauaaaa |
| | | ucugauaagaauguaauucuuaguaaccgaggacagagcgaagauagagaacagggcc |
| | | aauggccaagguggaaagguuugaaggaagcagcaugaaaccuacagacuuguaccaa |
| | | aauguucagugucaugaguguuaaaagugaaaagcuugcauguuaguauggauuucau |
| | | auccucggcagacagagcaccucacugugagugcgagaugaaguauucagaaaugaga |
| | | aacaacuacucaauuucaguguucauaucucaaauccaauaaacaacuuuagggguac |
| | | aauuuuuuuaaaaaauuacauuaaaauguuuuuaaaucucuucuuaauaauuuaaaaa |
| | | uuaaauugaaaauaacuuuaaaaaguaauauauacaggaaagccugugugcuaauuuu |
| | | uuagcgaggccauaaagcgagauaguugcucauuaauuucuacacaucagccuaucuu |
| | | uggcuucugccuugauagcgcacucugaauuaucuucuucauguucaucccucaucuu |
| | | uauuguuacugguuucauuucccuuggccacauagccccacuauuuuguauuccccaa |
| | | uggauauuguuccuuacaaaguucagccagcgcucagaaguacaaggaauuggcucuu |
| | | auacuucugucagacaggcaaaaacuucuaaaauuauacuauaauaaaaaucaaagag |
| | | augauauucauaauuaaacuaacaaaaguggcaggcccccccucccccaacaugagua |
| | | gaauuaaucugacguccauguucaagucugaaacacacuugccaauuaagagcacauu |
| | | agggccagccuuuaucucccucuuaguuacuaaugugcaguucaauggugagcuauag |
| | | agaaggaagccaagacuaccauaugucaaauauaaaaaaaaaaaaucccauuuuaaau |
| | | cuguagucccgaauuaaggacaagagagagggaaauaucuuugacauuagaaaaugga |
| | | gaaaauauuuuagcacaggacuuuacucagucacaucagaguugauaaguacguauga |
| | | caucccucuuuuuccuguuuuccugagaaaaugaucucucuaguguuucauuuaagau |
| | | aaguuuauugaauuaaacucaguaaaugaaacaacugacaugacuggagcuugaaaua |
| | | aacgaugugaugaucuacugaaauacaugaugcuaaauugucuugcuucuuaugcaaa |
| | | aacuacuauuaguuauagcaaugcauggauaauuaaggccaaaaauauauuagauguu |
| | | aaaaauaguuuuauauuuauacaucugaauuuuaauuuauauuuaaaguauauugguc |
| | | caaucaauucaugcccaaauguuuuaguucuauucuuugagauacuguuuuuguuuug |
| | | ggauuuuuuuuuaugagcuaaucucuugccuaggaguuccuacuucucucuccuccuu |
| | | uuauuuuuucuaauaaacuacacaugugucuucauccaggagcuaacuuucucccauu |
| | | uugcuuuuccuuuagcaccuuuuuuauauuagauuucuuucuuuucuccaucucuuug |
| | | cauauugccuauauuucuuuuccuaagcauaauauuuaaaaaagacuugaguuuuaug |
| | | uuaagauuauuucugcuuugcucuuacacagauaggauaaguagucuugauagaaaau |
| | | aaaucaaugauuccuagcgggaugucuuuuugcuuuuaaucaauaaggauucugacuu |
| | | cucuuucucuccauuuguguauuag |
| SEQ ID | Exon 22 pre- | GUGGUUGUGAAUGCCCUGUUAGGAGCAAUUCCAUCCAUCAUGAAUGUGCUUCUGGUUU |
| NO: 19 | mRNA | GCCUUAUAUUCUGGCUAAUUUUCAGCAUCAUGGGCGUAAAUUUGUUUGCUGGCAAAUU |
| | | CUACCACUGUGUUAACACCACAACUGGUGACAUAUUUGAGAUCAGCGAAGUCAAUAAU |
| | | CAUUCUGAUUGCCUAAAACUAAUAGAAAGAAAUGAGACCGCCCGGUGGAAAAAUGUGA |
| | | AAGUAAACUUUGAUAAUGUAGGAUUUGGGUAUCUUUCUUUGCUUCAAGUU |
| SEQ ID | Exon 21x pre- | gauaaucuugcuccaacuuggaugggguggagggugguuccccccucagcccuuuauu |
| NO: 20 | mRNA | augg |

### Example 14: SCN1A Exon 20x Region ASO Walk

An ASO walk was performed for SCN1A exon 20x region targeting sequences immediately upstream of 3′ splice site, across 3′splice site, exon 20x, across 5′ splice site, and downstream of 5′ splice site using 2′-MOE ASOs, PS backbone. ASOs were designed to cover these regions by shifting 5 nucleotides at a time. A list of ASOs targeting SCN1A is summarized in Table 3. Sequences of ASOs are summarized in Table 4A and Table 4B and Table 5A and Table 5B.

TABLE 3
List of ASOs targeting SCNIA.

| Gene | Pre-mRNA | ASOs | |
|---|---|---|---|
| SEQ ID NO: | SEQ ID NO: | SEQ ID NO: | NIE |
| SEQ ID NO: 1 | SEQ ID NO: 2 | SEQ ID NOS: | Exon 20x |
| | | 21-67, 210-256, 304-1099 | |
| SEQ ID NO: 11 | SEQ ID NO: 12 | SEQ ID NOs: | Exon 21x |
| | | 68-114, 257-303 | |

TABLE 4A
Sequences of ASOs targeting human SCN1A.

| SEQ ID NO: | Sequence name | ASO sequence |
|---|---|---|
| 21 | SCN1A-IVS19X−81 | GATGCTCTCCGTCTGTTT |
| 22 | SCN1A-IVS19X−76 | TTCATGATGCTCTCCGTC |
| 23 | SCN1A-IVS19X−71 | TTTTGTTCATGATGCTCT |
| 24 | SCN1A-IVS19X−66 | TTACTTTTTGTTCATGAT |
| 25 | SCN1A-IVS19X−61 | TGGTGTTACTTTTTGTTC |
| 26 | SCN1A-IVS19X−56 | ACATTTGGTGTTACTTTT |
| 27 | SCN1A-IVS19X−51 | ACAGAACATTTGGTGTTA |
| 28 | SCN1A-IVS19X−46 | ATATGACAGAACATTTGG |
| 29 | SCN1A-IVS19X−41 | ATCTGATATGACAGAACA |
| 30 | SCN1A-IVS19X−36 | TAGAAATCTGATATGACA |
| 31 | SCN1A-IVS19X−31 | TTAGTTAGAAATCTGATA |
| 32 | SCN1A-IVS19X−26 | TGTTATTAGTTAGAAATC |
| 33 | SCN1A-IVS19X−21 | TAGTTTGTTATTAGTTAG |
| 34 | SCN1A-IVS19X−16 | ATATATAGTTTGTTATTA |
| 35 | SCN1A-IVS19X−11 | TAGAAATATATAGTTTGT |
| 36 | SCN1A-IVS19X−6 | CAAAATAGAAATATATAG |
| 37 | SCN1A-IVS19X−3 | ATACAAAATAGAAATATA |
| 38 | SCN1A-IVS19X−1 | CTATACAAAATAGAAATA |
| 39 | SCN1A-I19X/E20X+2 | TCCTATACAAAATAGAAA |
| 40 | SCN1A-I19X/E20X+4 | TATCCTATACAAAATAGA |
| 41 | SCN1A-I19X/E20X+6 | ATTATCCTATACAAAATA |
| 42 | SCN1A-Ex20X+1 | AGTTGGAGCAAGATTATC |
| 43 | SCN1A-Ex20X+6 | ATCCAAGTTGGAGCAAGA |
| 44 | SCN1A-Ex20X+11 | ACCCCATCCAAGTTGGAG |
| 45 | SCN1A-Ex20X+16 | GCTCCACCCCATCCAAGT |
| 46 | SCN1A-Ex20X+21 | CCAGCGCTCCACCCCATC |
| 47 | SCN1A-Ex20X−24 | GAACCAGCGCTCCACCCC |
| 48 | SCN1A-Ex20X−19 | GGGAGGAACCAGCGCTCC |
| 49 | SCN1A-Ex20X−3 | ATAATAAAGGGCTCAGGG |
| 50 | SCN1A-Ex20X−1 | CCATAATAAAGGGCTCAG |
| 51 | SCN1A-E20X/I20X−6 | GTAATACAGTACCCATAA |
| 52 | SCN1A-E20X/I20X−4 | GGGTAATACAGTACCCAT |
| 53 | SCN1A-IVS20X+13 | TTAAAGGTAGCAAAAGGG |
| 54 | SCN1A-IVS20X+18 | AAGGATTAAAGGTAGCAA |
| 55 | SCN1A-IVS20X+23 | AGTGCAAGGATTAAAGGT |
| 56 | SCN1A-IVS20X+28 | GTCACAGTGCAAGGATTA |
| 57 | SCN1A-IVS20X+33 | CATAAGTCACAGTGCAAG |
| 58 | SCN1A-IVS20X+38 | CTACACATAAGTCACAGT |
| 59 | SCN1A-IVS20X+43 | CCCCACTACACATAAGTC |
| 60 | SCN1A-IVS20X+48 | CCTCACCCCACTACACAT |
| 61 | SCN1A-IVS20X+53 | CCCTCCCTCACCCCACTA |
| 62 | SCN1A-IVS20X+58 | CCAATCCCTCCCTCACCC |
| 63 | SCN1A-IVS20X+63 | CCTTCCCAATCCCTCCCT |
| 64 | SCN1A-IVS20X+68 | AGTACCCTTCCCAATCCC |
| 65 | SCN1A-IVS20X+73 | ATAATAGTACCCTTCCCA |
| 66 | SCN1A-IVS20X+78 | GTGCAATAATAGTACCCT |
| 67 | SCN1A-IVS20X+83 | CTGTGGTGCAATAATAGT |

TABLE 4B
Sequences of ASOs targeting human SCN1A.

| SEQ ID NO: | Sequence name | ASO sequence |
|---|---|---|
| 210 | SCN1A-IVS19X−81 | GAUGCUCUCCGUCUGUUU |
| 211 | SCN1A-IVS19X−76 | UUCAUGAUGCUCUCCGUC |
| 212 | SCN1A-IVS19X−71 | UUUUGUUCAUGAUGCUCU |
| 213 | SCN1A-IVS19X−66 | UUACUUUUUGUUCAUGAU |
| 214 | SCN1A-IVS19X−61 | UGGUGUUACUUUUUGUUC |
| 215 | SCN1A-IVS19X−56 | ACAUUUGGUGUUACUUUU |
| 216 | SCN1A-IVS19X−51 | ACAGAACAUUUGGUGUUA |
| 217 | SCN1A-IVS19X−46 | AUAUGACAGAACAUUUGG |
| 218 | SCN1A-IVS19X−41 | AUCUGAUAUGACAGAACA |
| 219 | SCN1A-IVS19X−36 | UAGAAAUCUGAUAUGACA |
| 220 | SCN1A-IVS19X−31 | UUAGUUAGAAAUCUGAUA |
| 221 | SCN1A-IVS19X−26 | UGUUAUUAGUUAGAAAUC |
| 222 | SCN1A-IVS19X−21 | UAGUUUGUUAUUAGUUAG |
| 223 | SCN1A-IVS19X−16 | AUAUAUAGUUUGUUAUUA |
| 224 | SCN1A-IVS19X−11 | UAGAAAUAUAUAGUUUGU |
| 225 | SCN1A-IVS19X−6 | CAAAAUAGAAAUAUAUAG |
| 226 | SCN1A-IVS19X−3 | AUACAAAAUAGAAAUAUA |
| 227 | SCN1A-IVS19X−1 | CUAUACAAAAUAGAAAUA |
| 228 | SCN1A-I19X/E20X+2 | UCCUAUACAAAAUAGAAA |
| 229 | SCN1A-I19X/E20X+4 | UAUCCUAUACAAAAUAGA |
| 230 | SCN1A-I19X/E20X+6 | AUUAUCCUAUACAAAAUA |
| 231 | SCN1A-Ex20X+1 | AGUUGGAGCAAGAUUAUC |
| 232 | SCN1A-Ex20X+6 | AUCCAAGUUGGAGCAAGA |
| 233 | SCN1A-Ex20X+11 | ACCCCAUCCAAGUUGGAG |
| 234 | SCN1A-Ex20X+16 | GCUCCACCCCAUCCAAGU |
| 235 | SCN1A-Ex20X+21 | CCAGCGCUCCACCCCAUC |
| 236 | SCN1A-Ex20X−24 | GAACCAGCGCUCCACCCC |
| 237 | SCN1A-Ex20X−19 | GGGAGGAACCAGCGCUCC |
| 238 | SCN1A-Ex20X−3 | AUAAUAAAGGGCUCAGGG |
| 239 | SCN1A-Ex20X−1 | CCAUAAUAAAGGGCUCAG |
| 240 | SCN1A-E20X/120X−6 | GUAAUACAGUACCCAUAA |
| 241 | SCN1A-E20X/120X−4 | GGGUAAUACAGUACCCAU |
| 242 | SCN1A-IVS20X+13 | UUAAAGGUAGCAAAAGGG |
| 243 | SCN1A-IVS20X+18 | AAGGAUUAAAGGUAGCAA |
| 244 | SCN1A-IVS20X+23 | AGUGCAAGGAUUAAAGGU |
| 245 | SCN1A-IVS20X+28 | GUCACAGUGCAAGGAUUA |
| 246 | SCN1A-IVS20X+33 | CAUAAGUCACAGUGCAAG |
| 247 | SCN1A-IVS20X+38 | CUACACAUAAGUCACAGU |
| 248 | SCN1A-IVS20X+43 | CCCCACUACACAUAAGUC |
| 249 | SCN1A-IVS20X+48 | CCUCACCCCACUACACAU |
| 250 | SCN1A-IVS20X+53 | CCCUCCCUCACCCCACUA |
| 251 | SCN1A-IVS20X+58 | CCAAUCCCUCCCUCACCC |
| 252 | SCN1A-IVS20X+63 | CCUUCCCAAUCCCUCCCU |
| 253 | SCN1A-IVS20X+68 | AGUACCCUUCCCAAUCCC |
| 254 | SCN1A-IVS20X+73 | AUAAUAGUACCCUUCCCA |
| 255 | SCN1A-IVS20X+78 | GUGCAAUAAUAGUACCCU |
| 256 | SCN1A-IVS20X+83 | CUGUGGUGCAAUAAUAGU |

TABLE 5A
Sequences of ASOs targeting mouse SCN1A.

| SEQ | | |
|---|---|---|
| ID NO: | Sequence name | ASO sequence |
| 68 | mScn1a-IVS20X − 81 | GATGCTCACTGCCTGTTT |
| 69 | mScn1a-IVS20X − 76 | TATATGATGCTCACTGCC |
| 70 | mScn1a-IVS20X − 71 | TTTTGTATATGATGCTCA |
| 71 | mScn1a-IVS20X − 66 | TTACTTTTTGTATATGAT |
| 72 | mScn1a-IVS20X − 61 | TGGTGTTACTTTTTGTAT |
| 73 | mScn1a-IVS20X − 56 | ACATTTGGTGTTACTTTT |
| 74 | mScn1a-IVS20X − 51 | ACAGAACATTTGGTGTTA |
| 75 | mScn1a-IVS20X − 46 | ATATGACAGAACATTTGG |
| 76 | mScn1a-IVS20X − 41 | AGCTGATATGACAGAACA |
| 77 | mScn1a-IVS20X − 36 | TAGAAAGCTGATATGACA |
| 78 | mScn1a-IVS20X − 31 | TTAGTTAGAAAGCTGATA |
| 79 | mScn1a-IVS20X − 26 | TATTATTAGTTAGAAAGC |
| 80 | mScn1a-IVS20X − 21 | TAGTTTATTATTAGTTAG |
| 81 | mScn1a-IVS20X − 16 | ATATATAGTTTATTATTA |
| 82 | mScn1a-IVS20X − 11 | TAGAAATATATAGTTTAT |
| 83 | mScn1a-IVS20X − 6 | TAAAATAGAAATATATAG |
| 84 | mScn1a-IVS20X − 3 | ATATAAAATAGAAATATA |
| 85 | mScn1a-IVS20X − 1 | CTATATAAAATAGAAATA |
| 86 | mScn1a-I20X/E21X + 2 | TCCTATATAAAATAGAAA |
| 87 | mScn1a-I20X/E21X + 4 | TATCCTATATAAAATAGA |
| 88 | mScn1a-I20X/E21X + 6 | ATTATCCTATATAAAATA |
| 89 | mScn1a-Ex21X + 1 | AGTTGGAGCAAGATTATC |
| 90 | mScn1a-Ex21X + 6 | ATCCAAGTTGGAGCAAGA |
| 91 | mScn1a-Ex21X + 11 | ACCCCATCCAAGTTGGAG |
| 92 | mScn1a-Ex21X + 16 | GCTCCACCCCATCCAAGT |
| 93 | mScn1a-Ex21X + 21 | CCACCGCTCCACCCCATC |
| 94 | mScn1a-Ex21X − 24 | GAACCACCGCTCCACCCC |
| 95 | mScn1a-Ex21X − 19 | GGGAGGAACCACCGCTCC |
| 96 | mScn1a-Ex21X − 3 | ATAATAAAGGGCTGAGGG |
| 97 | mScn1a-Ex21X − 1 | CCATAATAAAGGGCTGAG |
| 98 | mScn1a-E21X/I21X − 6 | GTAATACAGTACCCATAA |
| 99 | mScn1a-E21X/I21X − 4 | GGGTAATACAGTACCCAT |
| 100 | mScn1a-IVS21X + 13 | TTAAAGGTAGCAAAAGGG |
| 101 | mScn1a-IVS21X + 18 | AAGGATTAAAGGTAGCAA |
| 102 | mScn1a-IVS21X + 23 | AGTGCAAGGATTAAAGGT |
| 103 | mScn1a-IVS21X + 28 | GTCACAGTGCAAGGATTA |
| 104 | mScn1a-IVS21X + 33 | CATAAGTCACAGTGCAAG |
| 105 | mScn1a-IVS21X + 38 | CTACACATAAGTCACAGT |
| 106 | mScn1a-IVS21X + 43 | TCCCACTACACATAAGTC |
| 107 | mScn1a-IVS21X + 48 | CTCAATCCCACTACACAT |
| 108 | mScn1a-IVS21X + 53 | CCTCCCTCAATCCCACTA |
| 109 | mScn1a-IVS21X + 58 | CACTCCCTCCCTCAATCC |
| 110 | mScn1a-IVS21X + 63 | CTTCCCACTCCCTCCCTC |
| 111 | mScn1a-IVS21X + 68 | GTACCCTTCCCACTCCCT |
| 112 | mScn1a-IVS21X + 73 | CAATTGTACCCTTCCCAC |
| 113 | mScn1a-IVS21X + 78 | TGGTGCAATTGTACCCTT |
| 114 | mScn1a-IVS21X + 83 | TACTGTGGTGCAATTGTA |

TABLE 5B
Sequences of ASOs targeting mouse SCN1A.

| SEQ | | |
|---|---|---|
| ID NO: | Sequence name | ASO sequence |
| 257 | mScn1a-IVS20X − 81 | GAUGCUCACUGCCUGUUU |
| 258 | mScn1a-IVS20X − 76 | UAUAUGAUGCUCACUGCC |
| 259 | mScn1a-IVS20X − 71 | UUUUGUAUAUGAUGCUCA |
| 260 | mScn1a-IVS20X − 66 | UUACUUUUUGUAUAUGAU |
| 261 | mScn1a-IVS20X − 61 | UGGUGUUACUUUUUGUAU |
| 262 | mScn1a-IVS20X − 56 | ACAUUUGGUGUUACUUUU |
| 263 | mScn1a-IVS20X − 51 | ACAGAACAUUUGGUGUUA |
| 264 | mScn1a-IVS20X − 46 | AUAUGACAGAACAUUUGG |
| 265 | mScn1a-IVS20X − 41 | AGCUGAUAUGACAGAACA |
| 266 | mScn1a-IVS20X − 36 | UAGAAAGCUGAUAUGACA |
| 267 | mScn1a-IVS20X − 31 | UUAGUUAGAAAGCUGAUA |
| 268 | mScn1a-IVS20X − 26 | UAUUAUUAGUUAGAAAGC |
| 269 | mScn1a-IVS20X − 21 | UAGUUUAUUAUUAGUUAG |
| 270 | mScn1a-IVS20X − 16 | AUAUAUAGUUUAUUAUUA |
| 271 | mScn1a-IVS20X − 11 | UAGAAAUAUAUAGUUUAU |
| 272 | mScn1a-IVS20X − 6 | UAAAAUAGAAAUAUAUAG |
| 273 | mScn1a-IVS20X − 3 | AUAUAAAAUAGAAAUAUA |
| 274 | mScn1a-IVS20X − 1 | CUAUAUAAAAUAGAAAUA |
| 275 | mScn1a-I20X/E21X + 2 | UCCUAUAUAAAAUAGAAA |
| 276 | mScn1a-I20X/E21X + 4 | UAUCCUAUAUAAAAUAGA |
| 277 | mScn1a-I20X/E21X + 6 | AUUAUCCUAUAUAAAAUA |
| 278 | mScn1a-Ex21X + 1 | AGUUGGAGCAAGAUUAUC |
| 279 | mScn1a-Ex21X + 6 | AUCCAAGUUGGAGCAAGA |
| 280 | mScn1a-Ex21X + 11 | ACCCCAUCCAAGUUGGAG |
| 281 | mScn1a-Ex21X + 16 | GCUCCACCCCAUCCAAGU |
| 282 | mScn1a-Ex21X + 21 | CCACCGCUCCACCCCAUC |
| 283 | mScn1a-Ex21X − 24 | GAACCACCGCUCCACCCC |
| 284 | mScn1a-Ex21X − 19 | GGGAGGAACCACCGCUCC |
| 285 | mScn1a-Ex21X − 3 | AUAAUAAAGGGCUGAGGG |
| 286 | mScn1a-Ex21X − 1 | CCAUAAUAAAGGGCUGAG |
| 287 | mScn1a-E21X/121X − 6 | GUAAUACAGUACCCAUAA |
| 288 | mScn1a-E21X/121X − 4 | GGGUAAUACAGUACCCAU |
| 289 | mScn1a-IVS21X + 13 | UUAAAGGUAGCAAAAGGG |
| 290 | mScn1a-IVS21X + 18 | AAGGAUUAAAGGUAGCAA |
| 291 | mScn1a-IVS21X + 23 | AGUGCAAGGAUUAAAGGU |
| 292 | mScn1a-IVS21X + 28 | GUCACAGUGCAAGGAUUA |
| 293 | mScn1a-IVS21X + 33 | CAUAAGUCACAGUGCAAG |
| 294 | mScn1a-IVS21X + 38 | CUACACAUAAGUCACAGU |
| 295 | mScn1a-IVS21X + 43 | UCCCACUACACAUAAGUC |
| 296 | mScn1a-IVS21X + 48 | CUCAAUCCCACUACACAU |
| 297 | mScn1a-IVS21X + 53 | CCUCCCUCAAUCCCACUA |
| 298 | mScn1a-IVS21X + 58 | CACUCCCUCCCUCAAUCC |
| 299 | mScn1a-IVS21X + 63 | CUUCCCACUCCCUCCCUC |
| 300 | mScn1a-IVS21X + 68 | GUACCCUUCCCACUCCCU |
| 301 | mScn1a-IVS21X + 73 | CAAUUGUACCCUUCCCAC |
| 302 | mScn1a-IVS21X + 78 | UGGUGCAAUUGUACCCUU |
| 303 | mScn1a-IVS21X + 83 | UACUGUGGUGCAAUUGUA |

Sequences of ASOs are summarized in Table 6A and Table 6B.

TABLE 6A
Sequences of ASOs targeting human SCN1A.

| ASO ID | Sequence 5′-3′ | SEQ ID NO: |
|---|---|---|
| 1 | TTGGAGCAAGATTATC | 304 |
| 2 | GTTGGAGCAAGATTATC | 305 |
| 3 | GTTGGAGCAAGATTAT | 306 |
| 4 | AGTTGGAGCAAGATTAT | 307 |
| 5 | AGTTGGAGCAAGATTA | 308 |
| 6 | GATTATCCTATACAAAAT | 309 |
| 7 | AGATTATCCTATACAAAA | 310 |
| 8 | AAGATTATCCTATACAAA | 311 |
| 9 | CAAGATTATCCTATACAA | 312 |
| 10 | GCAAGATTATCCTATACA | 313 |
| 11 | AGCAAGATTATCCTATAC | 314 |
| 12 | GAGCAAGATTATCCTATA | 315 |
| 13 | GGAGCAAGATTATCCTAT | 316 |
| 14 | TGGAGCAAGATTATCCTA | 317 |
| 15 | GTTGGAGCAAGATTATCC | 318 |
| 16 | TTGGAGCAAGATTATCCT | 319 |
| 18 | AAGTTGGAGCAAGATTAT | 320 |
| 19 | CAAGTTGGAGCAAGATTA | 321 |
| 20 | CCAAGTTGGAGCAAGATT | 322 |
| 21 | TCCAAGTTGGAGCAAGAT | 323 |
| 22 | AGTACCCATAATAAAGGG | 324 |
| 23 | AATACAGTACCCATAATA | 325 |
| 24 | ATTAAAGGTAGCAAAAGG | 326 |
| 25 | GATTAAAGGTAGCAAAAG | 327 |
| 26 | GGATTAAAGGTAGCAAAA | 328 |
| 27 | AGGATTAAAGGTAGCAAA | 329 |
| 29 | CAAGGATTAAAGGTAGCA | 330 |
| 30 | GCAAGGATTAAAGGTAGC | 331 |
| 31 | TGCAAGGATTAAAGGTAG | 332 |
| 32 | GTGCAAGGATTAAAGGTA | 333 |
| 33 | AGTCACAGTGCAAGGATT | 334 |
| 34 | AAGTCACAGTGCAAGGAT | 335 |
| 35 | TAAGTCACAGTGCAAGGA | 336 |
| 36 | ATAAGTCACAGTGCAAGG | 337 |
| 38 | ACATAAGTCACAGTGCAA | 338 |
| 39 | CACATAAGTCACAGTGCA | 339 |
| 40 | ACACATAAGTCACAGTGC | 340 |
| 41 | TACACATAAGTCACAGTG | 341 |

TABLE 6B
Sequences of ASOs targeting human SCN1A.

| ASO ID | Sequence 5′-3′ | SEQ ID NO: |
|---|---|---|
| 1_U | UUGGAGCAAGAUUAUC | 342 |
| 2_U | GUUGGAGCAAGAUUAUC | 343 |
| 3_U | GUUGGAGCAAGAUUAU | 344 |
| 4_U | AGUUGGAGCAAGAUUAU | 345 |
| 5_U | AGUUGGAGCAAGAUUA | 346 |
| 6_U | GAUUAUCCUAUACAAAAU | 347 |
| 7_U | AGAUUAUCCUAUACAAAA | 348 |
| 8_U | AAGAUUAUCCUAUACAAA | 349 |
| 9_U | CAAGAUUAUCCUAUACAA | 350 |
| 10_U | GCAAGAUUAUCCUAUACA | 351 |
| 11_U | AGCAAGAUUAUCCUAUAC | 352 |
| 12_U | GAGCAAGAUUAUCCUAUA | 353 |
| 13_U | GGAGCAAGAUUAUCCUAU | 354 |
| 14_U | UGGAGCAAGAUUAUCCUA | 355 |
| 15_U | GUUGGAGCAAGAUUAUCC | 356 |
| 16_U | UUGGAGCAAGAUUAUCCU | 357 |
| 18_U | AAGUUGGAGCAAGAUUAU | 358 |
| 19_U | CAAGUUGGAGCAAGAUUA | 359 |
| 20_U | CCAAGUUGGAGCAAGAUU | 360 |
| 21_U | UCCAAGUUGGAGCAAGAU | 361 |
| 22_U | AGUACCCAUAAUAAAGGG | 362 |
| 23_U | AAUACAGUACCCAUAAUA | 363 |
| 24_U | AUUAAAGGUAGCAAAAGG | 364 |
| 25_U | GAUUAAAGGUAGCAAAAG | 365 |
| 26_U | GGAUUAAAGGUAGCAAAA | 366 |
| 27_U | AGGAUUAAAGGUAGCAAA | 367 |
| 29_U | CAAGGAUUAAAGGUAGCA | 368 |
| 30_U | GCAAGGAUUAAAGGUAGC | 369 |
| 31_U | UGCAAGGAUUAAAGGUAG | 370 |
| 32_U | GUGCAAGGAUUAAAGGUA | 371 |
| 33_U | AGUCACAGUGCAAGGAUU | 372 |
| 34_U | AAGUCACAGUGCAAGGAU | 373 |
| 35_U | UAAGUCACAGUGCAAGGA | 374 |
| 36_U | AUAAGUCACAGUGCAAGG | 375 |
| 38_U | ACAUAAGUCACAGUGCAA | 376 |
| 39_U | CACAUAAGUCACAGUGCA | 377 |
| 40_U | ACACAUAAGUCACAGUGC | 378 |
| 41_U | UACACAUAAGUCACAGUG | 379 |

Sequences of ASOs are summarized in Table 7.

TABLE 7
Sequences of ASOs targeting human SCN1A.

| Sequence | Chr2 | Chr2 | SEQ | | SEQ | |
|---|---|---|---|---|---|---|
| Name | Start | End | ID NO: | ASO sequence | ID NO: | ASO sequence |
| SCN1A- | 16686 | 16686 | 380 | ATGAATTTAATA | 740 | AUGAAUUUAA |
| IVS22-986 | 4788 | 4806 | | AACTTT | | UAAACUUU |
| SCN1A- | 16686 | 16686 | 381 | GACCAATGAAT | 741 | GACCAAUGAAU |
| IVS22-981 | 4783 | 4801 | | TTAATAA | | UUAAUAA |
| SCN1A- | 16686 | 16686 | 382 | CACTTGACCAA | 742 | CACUUGACCAA |
| IVS22-976 | 4778 | 4796 | | TGAATTT | | UGAAUUU |
| SCN1A- | 16686 | 16686 | 383 | CTGAGCACTTG | 743 | CUGAGCACUUG |
| IVS22-971 | 4773 | 4791 | | ACCAATG | | ACCAAUG |
| SCN1A- | 16686 | 16686 | 384 | AATATCTGAGC | 744 | AAUAUCUGAGC |
| IVS22-966 | 4768 | 4786 | | ACTTGAC | | ACUUGAC |
| SCN1A- | 16686 | 16686 | 385 | ATGGAAATATC | 745 | AUGGAAAUAU |
| IVS22-961 | 4763 | 4781 | | TGAGCAC | | CUGAGCAC |
| SCN1A- | 16686 | 16686 | 386 | AATGTATGGAA | 746 | AAUGUAUGGA |
| IVS22-956 | 4758 | 4776 | | ATATCTG | | AAUAUCUG |
| SCN1A- | 16686 | 16686 | 387 | AGTGTAATGTA | 747 | AGUGUAAUGU |
| IVS22-951 | 4753 | 4771 | | TGGAAAT | | AUGGAAAU |
| SCN1A- | 16686 | 16686 | 388 | AATGAAGTGTA | 748 | AAUGAAGUGU |
| IVS22-946 | 4748 | 4766 | | ATGTATG | | AAUGUAUG |
| SCN1A- | 16686 | 16686 | 389 | ATAGAAATGAA | 749 | AUAGAAAUGA |
| IVS22-941 | 4743 | 4761 | | GTGTAAT | | AGUGUAAU |
| SCN1A- | 16686 | 16686 | 390 | TTTTTATAGAA | 750 | UUUUUAUAGA |
| IVS22-936 | 4738 | 4756 | | ATGAAGT | | AAUGAAGU |
| SCN1A- | 16686 | 16686 | 391 | CAGCTTTTTTAT | 751 | CAGCUUUUUUA |
| IVS22-931 | 4733 | 4751 | | AGAAAT | | UAGAAAU |
| SCN1A- | 16686 | 16686 | 392 | AAGATCAGCTT | 752 | AAGAUCAGCUU |
| IVS22-926 | 4728 | 4746 | | TTTTATA | | UUUUAUA |
| SCN1A- | 16686 | 16686 | 393 | CCGATAAGATC | 753 | CCGAUAAGAUC |
| IVS22-921 | 4723 | 4741 | | AGCTTTT | | AGCUUUU |
| SCN1A- | 16686 | 16686 | 394 | GTATACCGATA | 754 | GUAUACCGAUA |
| IVS22-916 | 4718 | 4736 | | AGATCAG | | AGAUCAG |
| SCN1A- | 16686 | 16686 | 395 | TAAAAGTATAC | 755 | UAAAAGUAUA |
| IVS22-911 | 4713 | 4731 | | CGATAAG | | CCGAUAAG |
| SCN1A- | 16686 | 16686 | 396 | AAAATTAAAAG | 756 | AAAAUUAAAA |
| IVS22-906 | 4708 | 4726 | | TATACCG | | GUAUACCG |
| SCN1A- | 16686 | 16686 | 397 | CTGAGAAAATT | 757 | CUGAGAAAAU |
| IVS22-901 | 4703 | 4721 | | AAAAGTA | | UAAAAGUA |
| SCN1A- | 16686 | 16686 | 398 | TATTTCTGAGA | 758 | UAUUUCUGAG |
| IVS22-896 | 4698 | 4716 | | AAATTAA | | AAAAUUAA |
| SCN1A- | 16686 | 16686 | 399 | ATGGTTATTTCT | 759 | AUGGUUAUUU |
| IVS22-891 | 4693 | 4711 | | GAGAAA | | CUGAGAAA |
| SCN1A- | 16686 | 16686 | 400 | TAGATATGGTTA | 760 | UAGAUAUGGU |
| IVS22-886 | 4688 | 4706 | | TTTCTG | | UAUUUCUG |
| SCN1A- | 16686 | 16686 | 401 | AATTATAGATAT | 761 | AAUUAUAGAU |
| IVS22-881 | 4683 | 4701 | | GGTTAT | | AUGGUUAU |
| SCN1A- | 16686 | 16686 | 402 | TTAATAATTATA | 762 | UUAAUAAUUA |
| IVS22-876 | 4678 | 4696 | | GATATG | | UAGAUAUG |
| SCN1A- | 16686 | 16686 | 403 | ATTGATTAATAA | 763 | AUUGAUUAAU |
| IVS22-871 | 4673 | 4691 | | TTATAG | | AAUUAUAG |
| SCN1A- | 16686 | 16686 | 404 | GCATTATTGATT | 764 | GCAUUAUUGA |
| IVS22-866 | 4668 | 4686 | | AATAAT | | UUAAUAAU |
| SCN1A- | 16686 | 16686 | 405 | AAAAGGCATTA | 765 | AAAAGGCAUU |
| IVS22-861 | 4663 | 4681 | | TTGATTA | | AUUGAUUA |
| SCN1A- | 16686 | 16686 | 406 | AATATAAAAGG | 766 | AAUAUAAAAG |
| IVS22-856 | 4658 | 4676 | | CATTATT | | GCAUUAUU |
| SCN1A- | 16686 | 16686 | 407 | CTTTTAATATAA | 767 | CUUUUAAUAU |
| IVS22-851 | 4653 | 4671 | | AAGGCA | | AAAAGGCA |
| SCN1A- | 16686 | 16686 | 408 | AACCTCTTTTA | 768 | AACCUCUUUUA |
| IVS22-846 | 4648 | 4666 | | ATATAAA | | AUAUAAA |
| SCN1A- | 16686 | 16686 | 409 | AAACTAACCTC | 769 | AAACUAACCUC |
| IVS22-841 | 4643 | 4661 | | TTTTAAT | | UUUUAAU |
| SCN1A- | 16686 | 16686 | 410 | TTCAAAAACTA | 770 | UUCAAAAACUA |
| IVS22-836 | 4638 | 4656 | | ACCTCTT | | ACCUCUU |
| SCN1A- | 16686 | 16686 | 411 | CAAGTTTCAAA | 771 | CAAGUUUCAAA |
| IVS22-831 | 4633 | 4651 | | AACTAAC | | AACUAAC |
| SCN1A- | 16686 | 16686 | 412 | AACTCCAAGTT | 772 | AACUCCAAGUU |
| IVS22-826 | 4628 | 4646 | | TCAAAAA | | UCAAAAA |
| SCN1A- | 16686 | 16686 | 413 | TCTAAAACTCC | 773 | UCUAAAACUCC |
| IVS22-821 | 4623 | 4641 | | AAGTTTC | | AAGUUUC |
| SCN1A- | 16686 | 16686 | 414 | TTATGTCTAAA | 774 | UUAUGUCUAA |
| IVS22-816 | 4618 | 4636 | | ACTCCAA | | AACUCCAA |
| SCN1A- | 16686 | 16686 | 415 | GGATTTTATGTC | 775 | GGAUUUUAUG |
| IVS22-811 | 4613 | 4631 | | TAAAAC | | UCUAAAAC |
| SCN1A- | 16686 | 16686 | 416 | TATAAGGATTTT | 776 | UAUAAGGAUU |
| IVS22-806 | 4608 | 4626 | | ATGTCT | | UUAUGUCU |
| SCN1A- | 16686 | 16686 | 417 | GCATTTATAAG | 777 | GCAUUUAUAA |
| IVS22-801 | 4603 | 4621 | | GATTTTA | | GGAUUUUA |
| SCN1A- | 16686 | 16686 | 418 | TATCAGCATTTA | 778 | UAUCAGCAUUU |
| IVS22-796 | 4598 | 4616 | | TAAGGA | | AUAAGGA |
| SCN1A- | 16686 | 16686 | 419 | ATCACTATCAG | 779 | AUCACUAUCAG |
| IVS22-791 | 4593 | 4611 | | CATTTAT | | CAUUUAU |
| SCN1A- | 16686 | 16686 | 420 | GTTATATCACTA | 780 | GUUAUAUCACU |
| IVS22-786 | 4588 | 4606 | | TCAGCA | | AUCAGCA |
| SCN1A- | 16686 | 16686 | 421 | TATTAGTTATAT | 781 | UAUUAGUUAU |
| IVS22-781 | 4583 | 4601 | | CACTAT | | AUCACUAU |
| SCN1A- | 16686 | 16686 | 422 | TAAACTATTAG | 782 | UAAACUAUUA |
| IVS22-776 | 4578 | 4596 | | TTATATC | | GUUAUAUC |
| SCN1A- | 16686 | 16686 | 423 | CCATTTAAACT | 783 | CCAUUUAAACU |
| IVS22-771 | 4573 | 4591 | | ATTAGTT | | AUUAGUU |
| SCN1A- | 16686 | 16686 | 424 | TCTGACCATTT | 784 | UCUGACCAUUU |
| IVS22-766 | 4568 | 4586 | | AAACTAT | | AAACUAU |
| SCN1A- | 16686 | 16686 | 425 | ATAAATCTGAC | 785 | AUAAAUCUGAC |
| IVS22-761 | 4563 | 4581 | | CATTTAA | | CAUUUAA |
| SCN1A- | 16686 | 16686 | 426 | TATTCATAAATC | 786 | UAUUCAUAAA |
| IVS22-756 | 4558 | 4576 | | TGACCA | | UCUGACCA |
| SCN1A- | 16686 | 16686 | 427 | AGCCATATTCAT | 787 | AGCCAUAUUCA |
| IVS22-751 | 4553 | 4571 | | AAATCT | | UAAAUCU |
| SCN1A- | 16686 | 16686 | 428 | AATAGAGCCAT | 788 | AAUAGAGCCAU |
| IVS22-746 | 4548 | 4566 | | ATTCATA | | AUUCAUA |
| SCN1A- | 16686 | 16686 | 429 | TGAGGAATAGA | 789 | UGAGGAAUAG |
| IVS22-741 | 4543 | 4561 | | GCCATAT | | AGCCAUAU |
| SCN1A- | 16686 | 16686 | 430 | CATTATGAGGA | 790 | CAUUAUGAGG |
| IVS22-736 | 4538 | 4556 | | ATAGAGC | | AAUAGAGC |
| SCN1A- | 16686 | 16686 | 431 | GTTGTCATTATG | 791 | GUUGUCAUUA |
| IVS22-731 | 4533 | 4551 | | AGGAAT | | UGAGGAAU |
| SCN1A- | 16686 | 16686 | 432 | TGTATGTTGTC | 792 | UGUAUGUUGU |
| IVS22-726 | 4528 | 4546 | | ATTATGA | | CAUUAUGA |
| SCN1A- | 16686 | 16686 | 433 | CTGTGTGTATG | 793 | CUGUGUGUAU |
| IVS22-721 | 4523 | 4541 | | TTGTCAT | | GUUGUCAU |
| SCN1A- | 16686 | 16686 | 434 | TAGTGCTGTGT | 794 | UAGUGCUGUG |
| IVS22-716 | 4518 | 4536 | | GTATGTT | | UGUAUGUU |
| SCN1A- | 16686 | 16686 | 435 | CATTTTAGTGC | 795 | CAUUUUAGUGC |
| IVS22-711 | 4513 | 4531 | | TGTGTGT | | UGUGUGU |
| SCN1A- | 16686 | 16686 | 436 | TTAGTCATTTTA | 796 | UUAGUCAUUU |
| IVS22-706 | 4508 | 4526 | | GTGCTG | | UAGUGCUG |
| SCN1A- | 16686 | 16686 | 437 | AGAGATTAGTC | 797 | AGAGAUUAGU |
| IVS22-701 | 4503 | 4521 | | ATTTTAG | | CAUUUUAG |
| SCN1A- | 16686 | 16686 | 438 | ATTGAAGAGAT | 798 | AUUGAAGAGA |
| IVS22-696 | 4498 | 4516 | | TAGTCAT | | UUAGUCAU |
| SCN1A- | 16686 | 16686 | 439 | CACGTATTGAA | 799 | CACGUAUUGAA |
| IVS22-691 | 4493 | 4511 | | GAGATTA | | GAGAUUA |
| SCN1A- | 16686 | 16686 | 440 | CCAAACACGTA | 800 | CCAAACACGUA |
| IVS22-686 | 4488 | 4506 | | TTGAAGA | | UUGAAGA |
| SCN1A- | 16686 | 16686 | 441 | CAATGCCAAAC | 801 | CAAUGCCAAAC |
| IVS22-681 | 4483 | 4501 | | ACGTATT | | ACGUAUU |
| SCN1A- | 16686 | 16686 | 442 | CTCTACAATGC | 802 | CUCUACAAUGC |
| IVS22-676 | 4478 | 4496 | | CAAACAC | | CAAACAC |
| SCN1A- | 16686 | 16686 | 443 | TTTGACTCTAC | 803 | UUUGACUCUAC |
| IVS22-671 | 4473 | 4491 | | AATGCCA | | AAUGCCA |
| SCN1A- | 16686 | 16686 | 444 | GTTATTTTGACT | 804 | GUUAUUUUGA |
| IVS22-666 | 4468 | 4486 | | CTACAA | | CUCUACAA |
| SCN1A- | 16686 | 16686 | 445 | ATAACGTTATTT | 805 | AUAACGUUAU |
| IVS22-661 | 4463 | 4481 | | TGACTC | | UUUGACUC |
| SCN1A- | 16686 | 16686 | 446 | CAATTATAACG | 806 | CAAUUAUAACG |
| IVS22-656 | 4458 | 4476 | | TTATTTT | | UUAUUUU |
| SCN1A- | 16686 | 16686 | 447 | AGAATCAATTA | 807 | AGAAUCAAUU |
| IVS22-651 | 4453 | 4471 | | TAACGTT | | AUAACGUU |
| SCN1A- | 16686 | 16686 | 448 | AAAATAGAATC | 808 | AAAAUAGAAU |
| IVS22-646 | 4448 | 4466 | | AATTATA | | CAAUUAUA |
| SCN1A- | 16686 | 16686 | 449 | TATAAAAAATA | 809 | UAUAAAAAAU |
| IVS22-641 | 4443 | 4461 | | GAATCAA | | AGAAUCAA |
| SCN1A- | 16686 | 16686 | 450 | AGAAGTATAAA | 810 | AGAAGUAUAA |
| IVS22-636 | 4438 | 4456 | | AAATAGA | | AAAAUAGA |
| SCN1A- | 16686 | 16686 | 451 | ACACTAGAAGT | 811 | ACACUAGAAGU |
| IVS22-631 | 4433 | 4451 | | ATAAAAA | | AUAAAAA |
| SCN1A- | 16686 | 16686 | 452 | TCCAAACACTA | 812 | UCCAAACACUA |
| IVS22-626 | 4428 | 4446 | | GAAGTAT | | GAAGUAU |
| SCN1A- | 16686 | 16686 | 453 | AAATATCCAAA | 813 | AAAUAUCCAAA |
| IVS22-621 | 4423 | 4441 | | CACTAGA | | CACUAGA |
| SCN1A- | 16686 | 16686 | 454 | AAATAAAATAT | 814 | AAAUAAAAUA |
| IVS22-616 | 4418 | 4436 | | CCAAACA | | UCCAAACA |
| SCN1A- | 16686 | 16686 | 455 | TTACAAAATAA | 815 | UUACAAAAUA |
| IVS22-611 | 4413 | 4431 | | AATATCC | | AAAUAUCC |
| SCN1A- | 16686 | 16686 | 456 | TATTTTTACAAA | 816 | UAUUUUUACA |
| IVS22-606 | 4408 | 4426 | | ATAAAA | | AAAUAAAA |
| SCN1A- | 16686 | 16686 | 457 | GATTATATTTTT | 817 | GAUUAUAUUU |
| IVS22-601 | 4403 | 4421 | | ACAAAA | | UUACAAAA |
| SCN1A- | 16686 | 16686 | 458 | TTCATGATTATA | 818 | UUCAUGAUUA |
| IVS22-596 | 4398 | 4416 | | TTTTTA | | UAUUUUUA |
| SCN1A- | 16686 | 16686 | 459 | CATCATTCATG | 819 | CAUCAUUCAUG |
| IVS22-591 | 4393 | 4411 | | ATTATAT | | AUUAUAU |
| SCN1A- | 16686 | 16686 | 460 | CTCACCATCAT | 820 | CUCACCAUCAU |
| IVS22-586 | 4388 | 4406 | | TCATGAT | | UCAUGAU |
| SCN1A- | 16686 | 16686 | 461 | CCAACCTCACC | 821 | CCAACCUCACC |
| IVS22-581 | 4383 | 4401 | | ATCATTC | | AUCAUUC |
| SCN1A- | 16686 | 16686 | 462 | TATATCCAACCT | 822 | UAUAUCCAACC |
| IVS22-576 | 4378 | 4396 | | CACCAT | | UCACCAU |
| SCN1A- | 16686 | 16686 | 463 | ATTCTTATATCC | 823 | AUUCUUAUAUC |
| IVS22-571 | 4373 | 4391 | | AACCTC | | CAACCUC |
| SCN1A- | 16686 | 16686 | 464 | TCATCATTCTTA | 824 | UCAUCAUUCUU |
| IVS22-566 | 4368 | 4386 | | TATCCA | | AUAUCCA |
| SCN1A- | 16686 | 16686 | 465 | CATAATCATCAT | 825 | CAUAAUCAUCA |
| IVS22-561 | 4363 | 4381 | | TCTTAT | | UUCUUAU |
| SCN1A- | 16686 | 16686 | 466 | CCAATCATAAT | 826 | CCAAUCAUAAU |
| IVS22-556 | 4358 | 4376 | | CATCATT | | CAUCAUU |
| SCN1A- | 16686 | 16686 | 467 | ACTTCCCAATC | 827 | ACUUCCCAAUC |
| IVS22-551 | 4353 | 4371 | | ATAATCA | | AUAAUCA |
| SCN1A- | 16686 | 16686 | 468 | ATCTCACTTCC | 828 | AUCUCACUUCC |
| IVS22-546 | 4348 | 4366 | | CAATCAT | | CAAUCAU |
| SCN1A- | 16686 | 16686 | 469 | TTCAAATCTCA | 829 | UUCAAAUCUCA |
| IVS22-541 | 4343 | 4361 | | CTTCCCA | | CUUCCCA |
| SCN1A- | 16686 | 16686 | 470 | GCATGTTCAAA | 830 | GCAUGUUCAAA |
| IVS22-536 | 4338 | 4356 | | TCTCACT | | UCUCACU |
| SCN1A- | 16686 | 16686 | 471 | TCTGAGCATGT | 831 | UCUGAGCAUGU |
| IVS22-531 | 4333 | 4351 | | TCAAATC | | UCAAAUC |
| SCN1A- | 16686 | 16686 | 472 | GAGTTTCTGAG | 832 | GAGUUUCUGA |
| IVS22-526 | 4328 | 4346 | | CATGTTC | | GCAUGUUC |
| SCN1A- | 16686 | 16686 | 473 | AATGAGAGTTT | 833 | AAUGAGAGUU |
| IVS22-521 | 4323 | 4341 | | CTGAGCA | | UCUGAGCA |
| SCN1A- | 16686 | 16686 | 474 | AATTAAATGAG | 834 | AAUUAAAUGA |
| IVS22-516 | 4318 | 4336 | | AGTTTCT | | GAGUUUCU |
| SCN1A- | 16686 | 16686 | 475 | CAAAGAATTAA | 835 | CAAAGAAUUA |
| IVS22-511 | 4313 | 4331 | | ATGAGAG | | AAUGAGAG |
| SCN1A- | 16686 | 16686 | 476 | TAGGGCAAAG | 836 | UAGGGCAAAG |
| IVS22-506 | 4308 | 4326 | | AATTAAAT | | AAUUAAAU |
| SCN1A- | 16686 | 16686 | 477 | GCTGCTAGGGC | 837 | GCUGCUAGGGC |
| IVS22-501 | 4303 | 4321 | | AAAGAAT | | AAAGAAU |
| SCN1A- | 16686 | 16686 | 478 | TTTATGCTGCTA | 838 | UUUAUGCUGCU |
| IVS22-496 | 4298 | 4316 | | GGGCAA | | AGGGCAA |
| SCN1A- | 16686 | 16686 | 479 | GTGATTTTATGC | 839 | GUGAUUUUAU |
| IVS22-491 | 4293 | 4311 | | TGCTAG | | GCUGCUAG |
| SCN1A- | 16686 | 16686 | 480 | CTATTGTGATTT | 840 | CUAUUGUGAU |
| IVS22-486 | 4288 | 4306 | | TATGCT | | UUUAUGCU |
| SCN1A- | 16686 | 16686 | 481 | CGCAGCTATTG | 841 | CGCAGCUAUUG |
| IVS22-481 | 4283 | 4301 | | TGATTTT | | UGAUUUU |
| SCN1A- | 16686 | 16686 | 482 | TTTGACGCAGC | 842 | UUUGACGCAGC |
| IVS22-476 | 4278 | 4296 | | TATTGTG | | UAUUGUG |
| SCN1A- | 16686 | 16686 | 483 | TACGCTTTGAC | 843 | UACGCUUUGAC |
| IVS22-471 | 4273 | 4291 | | GCAGCTA | | GCAGCUA |
| SCN1A- | 16686 | 16686 | 484 | TGAGTTACGCT | 844 | UGAGUUACGCU |
| IVS22-466 | 4268 | 4286 | | TTGACGC | | UUGACGC |
| SCN1A- | 16686 | 16686 | 485 | GTGCCTGAGTT | 845 | GUGCCUGAGUU |
| IVS22-461 | 4263 | 4281 | | ACGCTTT | | ACGCUUU |
| SCN1A- | 16686 | 16686 | 486 | AATGAGTGCCT | 846 | AAUGAGUGCCU |
| IVS22-456 | 4258 | 4276 | | GAGTTAC | | GAGUUAC |
| SCN1A- | 16686 | 16686 | 487 | AATAAAATGAG | 847 | AAUAAAAUGA |
| IVS22-451 | 4253 | 4271 | | TGCCTGA | | GUGCCUGA |
| SCN1A- | 16686 | 16686 | 488 | ACAAAAATAAA | 848 | ACAAAAAUAA |
| IVS22-446 | 4248 | 4266 | | ATGAGTG | | AAUGAGUG |
| SCN1A- | 16686 | 16686 | 489 | GAACAACAAA | 849 | GAACAACAAAA |
| IVS22-441 | 4243 | 4261 | | AATAAAAT | | AUAAAAU |
| SCN1A- | 16686 | 16686 | 490 | TAACAGAACAA | 850 | UAACAGAACAA |
| IVS22-436 | 4238 | 4256 | | CAAAAAT | | CAAAAAU |
| SCN1A- | 16686 | 16686 | 491 | AAAAATAACAG | 851 | AAAAAUAACA |
| IVS22-431 | 4233 | 4251 | | AACAACA | | GAACAACA |
| SCN1A- | 16686 | 16686 | 492 | TTTGAAAAAAT | 852 | UUUGAAAAAA |
| IVS22-426 | 4228 | 4246 | | AACAGAA | | UAACAGAA |
| SCN1A- | 16686 | 16686 | 493 | CATGCTTTGAA | 853 | CAUGCUUUGAA |
| IVS22-421 | 4223 | 4241 | | AAAATAA | | AAAAUAA |
| SCN1A- | 16686 | 16686 | 494 | AAGCACATGCT | 854 | AAGCACAUGCU |
| IVS22-416 | 4218 | 4236 | | TTGAAAA | | UUGAAAA |
| SCN1A- | 16686 | 16686 | 495 | CATAAAAGCAC | 855 | CAUAAAAGCAC |
| IVS22-411 | 4213 | 4231 | | ATGCTTT | | AUGCUUU |
| SCN1A- | 16686 | 16686 | 496 | TGTTGCATAAA | 856 | UGUUGCAUAA |
| IVS22-406 | 4208 | 4226 | | AGCACAT | | AAGCACAU |
| SCN1A- | 16686 | 16686 | 497 | AGTAATGTTGC | 857 | AGUAAUGUUG |
| IVS22-401 | 4203 | 4221 | | ATAAAAG | | CAUAAAAG |
| SCN1A- | 16686 | 16686 | 498 | TATTCAGTAATG | 858 | UAUUCAGUAA |
| IVS22-396 | 4198 | 4216 | | TTGCAT | | UGUUGCAU |
| SCN1A- | 16686 | 16686 | 499 | TGCTTTATTCA | 859 | UGCUUUAUUCA |
| IVS22-391 | 4193 | 4211 | | GTAATGT | | GUAAUGU |
| SCN1A- | 16686 | 16686 | 500 | CAACATGCTTT | 860 | CAACAUGCUUU |
| IVS22-386 | 4188 | 4206 | | ATTCAGT | | AUUCAGU |
| SCN1A- | 16686 | 16686 | 501 | CTGTACAACAT | 861 | CUGUACAACAU |
| IVS22-381 | 4183 | 4201 | | GCTTTAT | | GCUUUAU |
| SCN1A- | 16686 | 16686 | 502 | AAGCACTGTAC | 862 | AAGCACUGUAC |
| IVS22-376 | 4178 | 4196 | | AACATGC | | AACAUGC |
| SCN1A- | 16686 | 16686 | 503 | TTATCAAGCAC | 863 | UUAUCAAGCAC |
| IVS22-371 | 4173 | 4191 | | TGTACAA | | UGUACAA |
| SCN1A- | 16686 | 16686 | 504 | ACTTCTTATCA | 864 | ACUUCUUAUCA |
| IVS22-366 | 4168 | 4186 | | AGCACTG | | AGCACUG |
| SCN1A- | 16686 | 16686 | 505 | TTCTAACTTCTT | 865 | UUCUAACUUCU |
| IVS22-361 | 4163 | 4181 | | ATCAAG | | UAUCAAG |
| SCN1A- | 16686 | 16686 | 506 | TTACTTTCTAAC | 866 | UUACUUUCUAA |
| IVS22-356 | 4158 | 4176 | | TTCTTA | | CUUCUUA |
| SCN1A- | 16686 | 16686 | 507 | ATTTGTTACTTT | 867 | AUUUGUUACU |
| IVS22-351 | 4153 | 4171 | | CTAACT | | UUCUAACU |
| SCN1A- | 16686 | 16686 | 508 | AATTTATTTGTT | 868 | AAUUUAUUUG |
| IVS22-346 | 4148 | 4166 | | ACTTTC | | UUACUUUC |
| SCN1A- | 16686 | 16686 | 509 | ATGATAATTTAT | 869 | AUGAUAAUUU |
| IVS22-341 | 4143 | 4161 | | TTGTTA | | AUUUGUUA |
| SCN1A- | 16686 | 16686 | 510 | ACGTGATGATA | 870 | ACGUGAUGAU |
| IVS22-336 | 4138 | 4156 | | ATTTATT | | AAUUUAUU |
| SCN1A- | 16686 | 16686 | 511 | GTGCAACGTGA | 871 | GUGCAACGUGA |
| IVS22-331 | 4133 | 4151 | | TGATAAT | | UGAUAAU |
| SCN1A- | 16686 | 16686 | 512 | ACAAAGTGCA | 872 | ACAAAGUGCAA |
| IVS22-326 | 4128 | 4146 | | ACGTGATG | | CGUGAUG |
| SCN1A- | 16686 | 16686 | 513 | AAAACACAAA | 873 | AAAACACAAAG |
| IVS22-321 | 4123 | 4141 | | GTGCAACG | | UGCAACG |
| SCN1A- | 16686 | 16686 | 514 | CATGCAAAACA | 874 | CAUGCAAAACA |
| IVS22-316 | 4118 | 4136 | | CAAAGTG | | CAAAGUG |
| SCN1A- | 16686 | 16686 | 515 | TAAAACATGCA | 875 | UAAAACAUGCA |
| IVS22-311 | 4113 | 4131 | | AAACACA | | AAACACA |
| SCN1A- | 16686 | 16686 | 516 | GTGCATAAAAC | 876 | GUGCAUAAAAC |
| IVS22-306 | 4108 | 4126 | | ATGCAAA | | AUGCAAA |
| SCN1A- | 16686 | 16686 | 517 | GAAATGTGCAT | 877 | GAAAUGUGCA |
| IVS22-301 | 4103 | 4121 | | AAAACAT | | UAAAACAU |
| SCN1A- | 16686 | 16686 | 518 | AGCCAGAAATG | 878 | AGCCAGAAAUG |
| IVS22-296 | 4098 | 4116 | | TGCATAA | | UGCAUAA |
| SCN1A- | 16686 | 16686 | 519 | CTGTCAGCCAG | 879 | CUGUCAGCCAG |
| IVS22-291 | 4093 | 4111 | | AAATGTG | | AAAUGUG |
| SCN1A- | 16686 | 16686 | 520 | AAAAGCTGTCA | 880 | AAAAGCUGUCA |
| IVS22-286 | 4088 | 4106 | | GCCAGAA | | GCCAGAA |
| SCN1A- | 16686 | 16686 | 521 | TGTTTAAAAGC | 881 | UGUUUAAAAG |
| IVS22-281 | 4083 | 4101 | | TGTCAGC | | CUGUCAGC |
| SCN1A- | 16686 | 16686 | 522 | ATAAATGTTTA | 882 | AUAAAUGUUU |
| IVS22-276 | 4078 | 4096 | | AAAGCTG | | AAAAGCUG |
| SCN1A- | 16686 | 16686 | 523 | ATACAATAAAT | 883 | AUACAAUAAA |
| IVS22-271 | 4073 | 4091 | | GTTTAAA | | UGUUUAAA |
| SCN1A- | 16686 | 16686 | 524 | TTGAAATACAA | 884 | UUGAAAUACA |
| IVS22-266 | 4068 | 4086 | | TAAATGT | | AUAAAUGU |
| SCN1A- | 16686 | 16686 | 525 | GAAATTTGAAA | 885 | GAAAUUUGAA |
| IVS22-261 | 4063 | 4081 | | TACAATA | | AUACAAUA |
| SCN1A- | 16686 | 16686 | 526 | GACTGGAAATT | 886 | GACUGGAAAU |
| IVS22-256 | 4058 | 4076 | | TGAAATA | | UUGAAAUA |
| SCN1A- | 16686 | 16686 | 527 | ATTTGGACTGG | 887 | AUUUGGACUG |
| IVS22-251 | 4053 | 4071 | | AAATTTG | | GAAAUUUG |
| SCN1A- | 16686 | 16686 | 528 | GAAAAATTTGG | 888 | GAAAAAUUUG |
| IVS22-246 | 4048 | 4066 | | ACTGGAA | | GACUGGAA |
| SCN1A- | 16686 | 16686 | 529 | AAGTTGAAAA | 889 | AAGUUGAAAA |
| IVS22-241 | 4043 | 4061 | | ATTTGGAC | | AUUUGGAC |
| SCN1A- | 16686 | 16686 | 530 | TTTACAAGTTG | 890 | UUUACAAGUU |
| IVS22-236 | 4038 | 4056 | | AAAAATT | | GAAAAAUU |
| SCN1A- | 16686 | 16686 | 531 | TTAATTTTACAA | 891 | UUAAUUUUAC |
| IVS22-231 | 4033 | 4051 | | GTTGAA | | AAGUUGAA |
| SCN1A- | 16686 | 16686 | 532 | TCAGTTTAATTT | 892 | UCAGUUUAAU |
| IVS22-226 | 4028 | 4046 | | TACAAG | | UUUACAAG |
| SCN1A- | 16686 | 16686 | 533 | TTCACTCAGTT | 893 | UUCACUCAGUU |
| IVS22-221 | 4023 | 4041 | | TAATTTT | | UAAUUUU |
| SCN1A- | 16686 | 16686 | 534 | ATCAATTCACT | 894 | AUCAAUUCACU |
| IVS22-216 | 4018 | 4036 | | CAGTTTA | | CAGUUUA |
| SCN1A- | 16686 | 16686 | 535 | ACGACATCAAT | 895 | ACGACAUCAAU |
| IVS22-211 | 4013 | 4031 | | TCACTCA | | UCACUCA |
| SCN1A- | 16686 | 16686 | 536 | TATTCACGACA | 896 | UAUUCACGACA |
| IVS22-206 | 4008 | 4026 | | TCAATTC | | UCAAUUC |
| SCN1A- | 16686 | 16686 | 537 | CTAGATATTCAC | 897 | CUAGAUAUUCA |
| IVS22-201 | 4003 | 4021 | | GACATC | | CGACAUC |
| SCN1A- | 16686 | 16686 | 538 | TTACCCTAGAT | 898 | UUACCCUAGAU |
| IVS22-196 | 3998 | 4016 | | ATTCACG | | AUUCACG |
| SCN1A- | 16686 | 16686 | 539 | TTATTTTACCCT | 899 | UUAUUUUACCC |
| IVS22-191 | 3993 | 4011 | | AGATAT | | UAGAUAU |
| SCN1A- | 16686 | 16686 | 540 | AAATTTTATTTT | 900 | AAAUUUUAUU |
| IVS22-186 | 3988 | 4006 | | ACCCTA | | UUACCCUA |
| SCN1A- | 16686 | 16686 | 541 | AACACAAATTT | 901 | AACACAAAUUU |
| IVS22-181 | 3983 | 4001 | | TATTTTA | | UAUUUUA |
| SCN1A- | 16686 | 16686 | 542 | ATTTAAACACA | 902 | AUUUAAACACA |
| IVS22-176 | 3978 | 3996 | | AATTTTA | | AAUUUUA |
| SCN1A- | 16686 | 16686 | 543 | TACAAATTTAA | 903 | UACAAAUUUA |
| IVS22-171 | 3973 | 3991 | | ACACAAA | | AACACAAA |
| SCN1A- | 16686 | 16686 | 544 | AAAAATACAAA | 904 | AAAAAUACAA |
| IVS22-166 | 3968 | 3986 | | TTTAAAC | | AUUUAAAC |
| SCN1A- | 16686 | 16686 | 545 | AAATTAAAAAT | 905 | AAAUUAAAAA |
| IVS22-161 | 3963 | 3981 | | ACAAATT | | UACAAAUU |
| SCN1A- | 16686 | 16686 | 546 | TTAGGAAATTA | 906 | UUAGGAAAUU |
| IVS22-156 | 3958 | 3976 | | AAAATAC | | AAAAAUAC |
| SCN1A- | 16686 | 16686 | 547 | CTAGGTTAGGA | 907 | CUAGGUUAGG |
| IVS22-151 | 3953 | 3971 | | AATTAAA | | AAAUUAAA |
| SCN1A- | 16686 | 16686 | 548 | ATTTCCTAGGT | 908 | AUUUCCUAGGU |
| IVS22-146 | 3948 | 3966 | | TAGGAAA | | UAGGAAA |
| SCN1A- | 16686 | 16686 | 549 | TTAAGATTTCC | 909 | UUAAGAUUUCC |
| IVS22-141 | 3943 | 3961 | | TAGGTTA | | UAGGUUA |
| SCN1A- | 16686 | 16686 | 550 | GGTATTTAAGA | 910 | GGUAUUUAAG |
| IVS22-136 | 3938 | 3956 | | TTTCCTA | | AUUUCCUA |
| SCN1A- | 16686 | 16686 | 551 | AAGAAGGTATT | 911 | AAGAAGGUAU |
| IVS22-131 | 3933 | 3951 | | TAAGATT | | UUAAGAUU |
| SCN1A- | 16686 | 16686 | 552 | TGAAAAAGAA | 912 | UGAAAAAGAA |
| IVS22-126 | 3928 | 3946 | | GGTATTTA | | GGUAUUUA |
| SCN1A- | 16686 | 16686 | 553 | TCTTTTGAAAA | 913 | UCUUUUGAAA |
| IVS22-121 | 3923 | 3941 | | AGAAGGT | | AAGAAGGU |
| SCN1A- | 16686 | 16686 | 554 | TGAGTTCTTTT | 914 | UGAGUUCUUU |
| IVS22-116 | 3918 | 3936 | | GAAAAAG | | UGAAAAAG |
| SCN1A- | 16686 | 16686 | 555 | AGACTTGAGTT | 915 | AGACUUGAGU |
| IVS22-111 | 3913 | 3931 | | CTTTTGA | | UCUUUUGA |
| SCN1A- | 16686 | 16686 | 556 | CATTAAGACTT | 916 | CAUUAAGACUU |
| IVS22-106 | 3908 | 3926 | | GAGTTCT | | GAGUUCU |
| SCN1A- | 16686 | 16686 | 557 | CTATCCATTAAG | 917 | CUAUCCAUUAA |
| IVS22-101 | 3903 | 3921 | | ACTTGA | | GACUUGA |
| SCN1A- | 16686 | 16686 | 558 | TTTCCCTATCCA | 918 | UUUCCCUAUCC |
| IVS22-096 | 3898 | 3916 | | TTAAGA | | AUUAAGA |
| SCN1A- | 16686 | 16686 | 559 | GTCTGTTTCCC | 919 | GUCUGUUUCCC |
| IVS22-091 | 3893 | 3911 | | TATCCAT | | UAUCCAU |
| SCN1A- | 16686 | 16686 | 560 | TTTCCCTACTG | 920 | UUUCCCUACUG |
| IVS23 + 091 | 3631 | 3649 | | TGGTGCA | | UGGUGCA |
| SCN1A- | 16686 | 16686 | 561 | TGTATTTTCCCT | 921 | UGUAUUUUCCC |
| IVS23 + 096 | 3626 | 3644 | | ACTGTG | | UACUGUG |
| SCN1A- | 16686 | 16686 | 562 | AATAATGTATTT | 922 | AAUAAUGUAU |
| IVS23 + 101 | 3621 | 3639 | | TCCCTA | | UUUCCCUA |
| SCN1A- | 16686 | 16686 | 563 | ATGTAAATAAT | 923 | AUGUAAAUAA |
| IVS23 + 106 | 3616 | 3634 | | GTATTTT | | UGUAUUUU |
| SCN1A- | 16686 | 16686 | 564 | TTAGGATGTAA | 924 | UUAGGAUGUA |
| IVS23 + 111 | 3611 | 3629 | | ATAATGT | | AAUAAUGU |
| SCN1A- | 16686 | 16686 | 565 | AGGGATTAGGA | 925 | AGGGAUUAGG |
| IVS23 + 116 | 3606 | 3624 | | TGTAAAT | | AUGUAAAU |
| SCN1A- | 16686 | 16686 | 566 | AAAGAGGGAA | 926 | AAAGAGGGAA |
| IVS23 + 121 | 3601 | 3619 | | TTAGGATG | | UUAGGAUG |
| SCN1A- | 16686 | 16686 | 567 | ATTGAAAAGAA | 927 | AUUGAAAAGA |
| IVS23 + 126 | 3596 | 3614 | | GGGATTA | | AGGGAUUA |
| SCN1A- | 16686 | 16686 | 568 | AGACAATTGAA | 928 | AGACAAUUGA |
| IVS23 + 131 | 3591 | 3609 | | AAGAGGG | | AAAGAGGG |
| SCN1A- | 16686 | 16686 | 569 | ATTTAAGACAA | 929 | AUUUAAGACA |
| IVS23 + 136 | 3586 | 3604 | | TTGAAAA | | AUUGAAAA |
| SCN1A- | 16686 | 16686 | 570 | ATGAAATTTAA | 930 | AUGAAAUUUA |
| IVS23 + 141 | 3581 | 3599 | | GACAATT | | AGACAAUU |
| SCN1A- | 16686 | 16686 | 571 | TTCAAATGAAA | 931 | UUCAAAUGAA |
| IVS23 + 146 | 3576 | 3594 | | TTTAAGA | | AUUUAAGA |
| SCN1A- | 16686 | 16686 | 572 | TTTTTTTCAAAT | 932 | UUUUUUUCAA |
| IVS23 + 151 | 3571 | 3589 | | GAAATT | | AUGAAAUU |
| SCN1A- | 16686 | 16686 | 573 | TTTTTTTTTTTT | 933 | UUUUUUUUUU |
| IVS23 + 156 | 3566 | 3584 | | CAAATG | | UUCAAAUG |
| SCN1A- | 16686 | 16686 | 574 | AAGGTTTTTTT | 934 | AAGGUUUUUU |
| IVS23 + 161 | 3561 | 3579 | | TTTTTTC | | UUUUUUUC |
| SCN1A- | 16686 | 16686 | 575 | TCATAAAGGTT | 935 | UCAUAAAGGU |
| IVS23 + 166 | 3556 | 3574 | | TTTTTTT | | UUUUUUUU |
| SCN1A- | 16686 | 16686 | 576 | TAAATTCATAA | 936 | UAAAUUCAUA |
| IVS23 + 171 | 3551 | 3569 | | AGGTTTT | | AAGGUUUU |
| SCN1A- | 16686 | 16686 | 577 | GAGGGTAAATT | 937 | GAGGGUAAAU |
| IVS23 + 176 | 3546 | 3564 | | CATAAAG | | UCAUAAAG |
| SCN1A- | 16686 | 16686 | 578 | CCACAGAGGGT | 938 | CCACAGAGGGU |
| IVS23 + 181 | 3541 | 3559 | | AAATTCA | | AAAUUCA |
| SCN1A- | 16686 | 16686 | 579 | AAAATCCACAG | 939 | AAAAUCCACAG |
| IVS23 + 186 | 3536 | 3554 | | AGGGTAA | | AGGGUAA |
| SCN1A- | 16686 | 16686 | 580 | GGGTTAAAATC | 940 | GGGUUAAAAU |
| IVS23 + 191 | 3531 | 3549 | | CACAGAG | | CCACAGAG |
| SCN1A- | 16686 | 16686 | 581 | CATTGGGATTA | 941 | CAUUGGGAUU |
| IVS23 + 196 | 3526 | 3544 | | AAATCCA | | AAAAUCCA |
| SCN1A- | 16686 | 16686 | 582 | TCAACCATTAG | 942 | UCAACCAUUAG |
| IVS23 + 201 | 3521 | 3539 | | GGTTAAA | | GGUUAAA |
| SCN1A- | 16686 | 16686 | 583 | AGATATCAACC | 943 | AGAUAUCAACC |
| IVS23 + 206 | 3516 | 3534 | | ATTGGGA | | AUUGGGA |
| SCN1A- | 16686 | 16686 | 584 | AATAAAGATAT | 944 | AAUAAAGAUA |
| IVS23 + 211 | 3511 | 3529 | | CAACCAT | | UCAACCAU |
| SCN1A- | 16686 | 16686 | 585 | AACTTAATAAA | 945 | AACUUAAUAA |
| IVS23 + 216 | 3506 | 3524 | | GATATCA | | AGAUAUCA |
| SCN1A- | 16686 | 16686 | 586 | AATGAAACTTA | 946 | AAUGAAACUU |
| IVS23 + 221 | 3501 | 3519 | | ATAAAGA | | AAUAAAGA |
| SCN1A- | 16686 | 16686 | 587 | TATTCAATGAA | 947 | UAUUCAAUGA |
| IVS23 + 226 | 3496 | 3514 | | ACTTAAT | | AACUUAAU |
| SCN1A- | 16686 | 16686 | 588 | AATCATATTCA | 948 | AAUCAUAUUCA |
| IVS23 + 231 | 3491 | 3509 | | ATGAAAC | | AUGAAAC |
| SCN1A- | 16686 | 16686 | 589 | AACTAAATCAT | 949 | AACUAAAUCAU |
| IVS23 + 236 | 3486 | 3504 | | ATTCAAT | | AUUCAAU |
| SCN1A- | 16686 | 16686 | 590 | CACATAACTAA | 950 | CACAUAACUAA |
| IVS23 + 241 | 3481 | 3499 | | ATCATAT | | AUCAUAU |
| SCN1A- | 16686 | 16686 | 591 | ATATACACATAA | 951 | AUAUACACAUA |
| IVS23 + 246 | 3476 | 3494 | | CTAAAT | | ACUAAAU |
| SCN1A- | 16686 | 16686 | 592 | ACTCCATATAC | 952 | ACUCCAUAUAC |
| IVS23 + 251 | 3471 | 3489 | | ACATAAC | | ACAUAAC |
| SCN1A- | 16686 | 16686 | 593 | GGATAACTCCA | 953 | GGAUAACUCCA |
| IVS23 + 256 | 3466 | 3484 | | TATACAC | | UAUACAC |
| SCN1A- | 16686 | 16686 | 594 | AAGATGGATAA | 954 | AAGAUGGAUA |
| IVS23 + 261 | 3461 | 3479 | | CTCCATA | | ACUCCAUA |
| SCN1A- | 16686 | 16686 | 595 | CCCCAAAGATG | 955 | CCCCAAAGAUG |
| IVS23 + 266 | 3456 | 3474 | | GATAACT | | GAUAACU |
| SCN1A- | 16686 | 16686 | 596 | AATCTCCCCAA | 956 | AAUCUCCCCAA |
| IVS23 + 271 | 3451 | 3469 | | AGATGGA | | AGAUGGA |
| SCN1A- | 16686 | 16686 | 597 | CCAGTAATCTC | 957 | CCAGUAAUCUC |
| IVS23 + 276 | 3446 | 3464 | | CCCAAAG | | CCCAAAG |
| SCN1A- | 16686 | 16686 | 598 | CCAATCCAGTA | 958 | CCAAUCCAGUA |
| IVS23 + 281 | 3441 | 3459 | | ATCTCCC | | AUCUCCC |
| SCN1A- | 16686 | 16686 | 599 | CCTCACCAATC | 959 | CCUCACCAAUC |
| IVS23 + 286 | 3436 | 3454 | | CAGTAAT | | CAGUAAU |
| SCN1A- | 16686 | 16686 | 600 | CCCGCCCTCAC | 960 | CCCGCCCUCAC |
| IVS23 + 291 | 3431 | 3449 | | CAATCCA | | CAAUCCA |
| SCN1A- | 16686 | 16686 | 601 | GGTCCCCCGCC | 961 | GGUCCCCCGCC |
| IVS23 + 296 | 3426 | 3444 | | CTCACCA | | CUCACCA |
| SCN1A- | 16686 | 16686 | 602 | ACCAGGGTCCC | 962 | ACCAGGGUCCC |
| IVS23 + 301 | 3421 | 3439 | | CCGCCCT | | CCGCCCU |
| SCN1A- | 16686 | 16686 | 603 | TCTACACCAGG | 963 | UCUACACCAGG |
| IVS23 + 306 | 3416 | 3434 | | GTCCCCC | | GUCCCCC |
| SCN1A- | 16686 | 16686 | 604 | ATCATTCTACA | 964 | AUCAUUCUACA |
| IVS23 + 311 | 3411 | 3429 | | CCAGGGT | | CCAGGGU |
| SCN1A- | 16686 | 16686 | 605 | ACATAATCATTC | 965 | ACAUAAUCAUU |
| IVS23 + 316 | 3406 | 3424 | | TACACC | | CUACACC |
| SCN1A- | 16686 | 16686 | 606 | TTTTCACATAAT | 966 | UUUUCACAUAA |
| IVS23 + 321 | 3401 | 3419 | | CATTCT | | UCAUUCU |
| SCN1A- | 16686 | 16686 | 607 | TTGTTTTTTCAC | 967 | UUGUUUUUUC |
| IVS23 + 326 | 3396 | 3414 | | ATAATC | | ACAUAAUC |
| SCN1A- | 16686 | 16686 | 608 | TTAAATTGTTTT | 968 | UUAAAUUGUU |
| IVS23 + 331 | 3391 | 3409 | | TTCACA | | UUUUCACA |
| SCN1A- | 16686 | 16686 | 609 | ACAAGTTAAAT | 969 | ACAAGUUAAA |
| IVS23 + 336 | 3386 | 3404 | | TGTTTTT | | UUGUUUUU |
| SCN1A- | 16686 | 16686 | 610 | GCTTAACAAGT | 970 | GCUUAACAAGU |
| IVS23 + 341 | 3381 | 3399 | | TAAATTG | | UAAAUUG |
| SCN1A- | 16686 | 16686 | 611 | CATGAGCTTAA | 971 | CAUGAGCUUAA |
| IVS23 + 346 | 3376 | 3394 | | CAAGTTA | | CAAGUUA |
| SCN1A- | 16686 | 16686 | 612 | AGTATCATGAG | 972 | AGUAUCAUGA |
| IVS23 + 351 | 3371 | 3389 | | CTTAACA | | GCUUAACA |
| SCN1A- | 16686 | 16686 | 613 | CAAACAGTATC | 973 | CAAACAGUAUC |
| IVS23 + 356 | 3366 | 3384 | | ATGAGCT | | AUGAGCU |
| SCN1A- | 16686 | 16686 | 614 | TGCCTCAAACA | 974 | UGCCUCAAACA |
| IVS23 + 361 | 3361 | 3379 | | GTATCAT | | GUAUCAU |
| SCN1A- | 16686 | 16686 | 615 | CTGTATGCCTC | 975 | CUGUAUGCCUC |
| IVS23 + 366 | 3356 | 3374 | | AAACAGT | | AAACAGU |
| SCN1A- | 16686 | 16686 | 616 | AGGGACTGTAT | 976 | AGGGACUGUA |
| IVS23 + 371 | 3351 | 3369 | | GCCTCAA | | UGCCUCAA |
| SCN1A- | 16686 | 16686 | 617 | ACAGCAGGGA | 977 | ACAGCAGGGAC |
| IVS23 + 376 | 3346 | 3364 | | CTGTATGC | | UGUAUGC |
| SCN1A- | 16686 | 16686 | 618 | ACTAAACAGCA | 978 | ACUAAACAGCA |
| IVS23 + 381 | 3341 | 3359 | | AGGGCTG | | AGGGCUG |
| SCN1A- | 16686 | 16686 | 619 | AATGTACTAAA | 979 | AAUGUACUAA |
| IVS23 + 386 | 3336 | 3354 | | CAGCAGG | | ACAGCAGG |
| SCN1A- | 16686 | 16686 | 620 | AGACCAATGTA | 980 | AGACCAAUGUA |
| IVS23 + 391 | 3331 | 3349 | | CTAAACA | | CUAAACA |
| SCN1A- | 16686 | 16686 | 621 | GACCCAGACCA | 981 | GACCCAGACCA |
| IVS23 + 396 | 3326 | 3344 | | ATGTACT | | AUGUACU |
| SCN1A- | 16686 | 16686 | 622 | TTCAGGACCCA | 982 | UUCAGGACCCA |
| IVS23 + 401 | 3321 | 3339 | | GACCAAT | | GACCAAU |
| SCN1A- | 16686 | 16686 | 623 | TAATTTTCAGG | 983 | UAAUUUUCAG |
| IVS23 + 406 | 3316 | 3334 | | ACCCAGA | | GACCCAGA |
| SCN1A- | 16686 | 16686 | 624 | ACTGGTAATTT | 984 | ACUGGUAAUU |
| IVS23 + 411 | 3311 | 3329 | | TCAGGAC | | UUCAGGAC |
| SCN1A- | 16686 | 16686 | 625 | ATCTAACTGGT | 985 | AUCUAACUGGU |
| IVS23 + 416 | 3306 | 3324 | | AATTTTC | | AAUUUUC |
| SCN1A- | 16686 | 16686 | 626 | ATGGTATCTAA | 986 | AUGGUAUCUA |
| IVS23 + 421 | 3301 | 3319 | | CTGGTAA | | ACUGGUAA |
| SCN1A- | 16686 | 16686 | 627 | AACTGATGGTA | 987 | AACUGAUGGU |
| IVS23 + 426 | 3296 | 3314 | | TCTAACT | | AUCUAACU |
| SCN1A- | 16686 | 16686 | 628 | TAATCAACTGA | 988 | UAAUCAACUGA |
| IVS23 + 431 | 3291 | 3309 | | TGGTATC | | UGGUAUC |
| SCN1A- | 16686 | 16686 | 629 | ATCAATAATCA | 989 | AUCAAUAAUCA |
| IVS23 + 436 | 3286 | 3304 | | ACTGATG | | ACUGAUG |
| SCN1A- | 16686 | 16686 | 630 | TACATATCAATA | 990 | UACAUAUCAAU |
| IVS23 + 441 | 3281 | 3299 | | ATCAAC | | AAUCAAC |
| SCN1A- | 16686 | 16686 | 631 | GCTCATACATAT | 991 | GCUCAUACAUA |
| IVS23 + 446 | 3276 | 3294 | | CAATAA | | UCAAUAA |
| SCN1A- | 16686 | 16686 | 632 | TATCTGCTCATA | 992 | UAUCUGCUCAU |
| IVS23 + 451 | 3271 | 3289 | | CATATC | | ACAUAUC |
| SCN1A- | 16686 | 16686 | 633 | CCTAGTATCTG | 993 | CCUAGUAUCUG |
| IVS23 + 456 | 3266 | 3284 | | CTCATAC | | CUCAUAC |
| SCN1A- | 16686 | 16686 | 634 | TGCACCCTAGT | 994 | UGCACCCUAGU |
| IVS23 + 461 | 3261 | 3279 | | ATCTGCT | | AUCUGCU |
| SCN1A- | 16686 | 16686 | 635 | AATATTGCACC | 995 | AAUAUUGCACC |
| IVS23 + 466 | 3256 | 3274 | | CTAGTAT | | CUAGUAU |
| SCN1A- | 16686 | 16686 | 636 | CCTGAAATATT | 996 | CCUGAAAUAUU |
| IVS23 + 471 | 3251 | 3269 | | GCACCCT | | GCACCCU |
| SCN1A- | 16686 | 16686 | 637 | TGAAACCTGAA | 997 | UGAAACCUGAA |
| IVS23 + 476 | 3246 | 3264 | | ATATTGC | | AUAUUGC |
| SCN1A- | 16686 | 16686 | 638 | TCTTATGAAAC | 998 | UCUUAUGAAAC |
| IVS23 + 481 | 3241 | 3259 | | CTGAAAT | | CUGAAAU |
| SCN1A- | 16686 | 16686 | 639 | ACCAGTCTTAT | 999 | ACCAGUCUUAU |
| IVS23 + 486 | 3236 | 3254 | | GAAACCT | | GAAACCU |
| SCN1A- | 16686 | 16686 | 640 | TCAATACCAGT | 1000 | UCAAUACCAGU |
| IVS23 + 491 | 3231 | 3249 | | CTTATGA | | CUUAUGA |
| SCN1A- | 16686 | 16686 | 641 | CACAATCAATA | 1001 | CACAAUCAAUA |
| IVS23 + 496 | 3226 | 3244 | | CCAGTCT | | CCAGUCU |
| SCN1A- | 16686 | 16686 | 642 | GTGGTCACAAT | 1002 | GUGGUCACAAU |
| IVS23 + 501 | 3221 | 3239 | | CAATACC | | CAAUACC |
| SCN1A- | 16686 | 16686 | 643 | TGAGAGTGGTC | 1003 | UGAGAGUGGU |
| IVS23 + 506 | 3216 | 3234 | | ACAATCA | | CACAAUCA |
| SCN1A- | 16686 | 16686 | 644 | AAAAATGAGA | 1004 | AAAAAUGAGA |
| IVS23 + 511 | 3211 | 3229 | | GTGGTCAC | | GUGGUCAC |
| SCN1A- | 16686 | 16686 | 645 | CAATAAAAAAT | 1005 | CAAUAAAAAA |
| IVS23 + 516 | 3206 | 3224 | | GAGAGTG | | UGAGAGUG |
| SCN1A- | 16686 | 16686 | 646 | TTACACAATAA | 1006 | UUACACAAUAA |
| IVS23 + 521 | 3201 | 3219 | | AAAATGA | | AAAAUGA |
| SCN1A- | 16686 | 16686 | 647 | TGAACTTACAC | 1007 | UGAACUUACAC |
| IVS23 + 526 | 3196 | 3214 | | AATAAAA | | AAUAAAA |
| SCN1A- | 16686 | 16686 | 648 | CCATATGAACT | 1008 | CCAUAUGAACU |
| IVS23 + 531 | 3191 | 3209 | | TACACAA | | UACACAA |
| SCN1A- | 16686 | 16686 | 649 | TAACCCCATAT | 1009 | UAACCCCAUAU |
| IVS23 + 536 | 3186 | 3204 | | GAACTTA | | GAACUUA |
| SCN1A- | 16686 | 16686 | 650 | GAAAATAACCC | 1010 | GAAAAUAACCC |
| IVS23 + 541 | 3181 | 3199 | | CATATGA | | CAUAUGA |
| SCN1A- | 16686 | 16686 | 651 | ATTTTGAAAAT | 1011 | AUUUUGAAAA |
| IVS23 + 546 | 3176 | 3194 | | AACCCCA | | UAACCCCA |
| SCN1A- | 16686 | 16686 | 652 | TTAACATTTTG | 1012 | UUAACAUUUU |
| IVS23 + 551 | 3171 | 3189 | | AAAATAA | | GAAAAUAA |
| SCN1A- | 16686 | 16686 | 653 | CCTTGTTAACA | 1013 | CCUUGUUAACA |
| IVS23 + 556 | 3166 | 3184 | | TTTTGAA | | UUUUGAA |
| SCN1A- | 16686 | 16686 | 654 | TTTTGCCTTGTT | 1014 | UUUUGCCUUGU |
| IVS23 + 561 | 3161 | 3179 | | AACATT | | UAACAUU |
| SCN1A- | 16686 | 16686 | 655 | TATATTTTTGCC | 1015 | UAUAUUUUUG |
| IVS23 + 566 | 3156 | 3174 | | TTGTTA | | CCUUGUUA |
| SCN1A- | 16686 | 16686 | 656 | CTTAATATATTT | 1016 | CUUAAUAUAU |
| IVS23 + 571 | 3151 | 3169 | | TTGCCT | | UUUUGCCU |
| SCN1A- | 16686 | 16686 | 657 | TATTTCTTAATA | 1017 | UAUUUCUUAA |
| IVS23 + 576 | 3146 | 3164 | | TATTTT | | UAUAUUUU |
| SCN1A- | 16686 | 16686 | 658 | TCAACTATTTCT | 1018 | UCAACUAUUUC |
| IVS23 + 581 | 3141 | 3159 | | TAATAT | | UUAAUAU |
| SCN1A- | 16686 | 16686 | 659 | CTTATTCAACTA | 1019 | CUUAUUCAACU |
| IVS23 + 586 | 3136 | 3154 | | TTTCTT | | AUUUCUU |
| SCN1A- | 16686 | 16686 | 660 | ATGTGCTTATTC | 1020 | AUGUGCUUAU |
| IVS23 + 591 | 3131 | 3149 | | AACTAT | | UCAACUAU |
| SCN1A- | 16686 | 16686 | 661 | TTCACATGTGC | 1021 | UUCACAUGUGC |
| IVS23 + 596 | 3126 | 3144 | | TTATTCA | | UUAUUCA |
| SCN1A- | 16686 | 16686 | 662 | CACAATTCACA | 1022 | CACAAUUCACA |
| IVS23 + 601 | 3121 | 3139 | | TGTGCTT | | UGUGCUU |
| SCN1A- | 16686 | 16686 | 663 | TACAACACAAT | 1023 | UACAACACAAU |
| IVS23 + 606 | 3116 | 3134 | | TCACATG | | UCACAUG |
| SCN1A- | 16686 | 16686 | 664 | TTGTTTACAAC | 1024 | UUGUUUACAAC |
| IVS23 + 611 | 3111 | 3129 | | ACAATTC | | ACAAUUC |
| SCN1A- | 16686 | 16686 | 665 | ACTTTTTGTTTA | 1025 | ACUUUUUGUU |
| IVS23 + 616 | 3106 | 3124 | | CAACAC | | UACAACAC |
| SCN1A- | 16686 | 16686 | 666 | TTCTAACTTTTT | 1026 | UUCUAACUUUU |
| IVS23 + 621 | 3101 | 3119 | | GTTTAC | | UGUUUAC |
| SCN1A- | 16686 | 16686 | 667 | TTTTATTCTAAC | 1027 | UUUUAUUCUA |
| IVS23 + 626 | 3096 | 3114 | | TTTTTG | | ACUUUUUG |
| SCN1A- | 16686 | 16686 | 668 | GATTTTTTTATT | 1028 | GAUUUUUUUA |
| IVS23 + 631 | 3091 | 3109 | | CTAACT | | UUCUAACU |
| SCN1A- | 16686 | 16686 | 669 | AAGTGGATTTT | 1029 | AAGUGGAUUU |
| IVS23 + 636 | 3086 | 3104 | | TTTATTC | | UUUUAUUC |
| SCN1A- | 16686 | 16686 | 670 | CAAATAAGTGG | 1030 | CAAAUAAGUG |
| IVS23 + 641 | 3081 | 3099 | | ATTTTTT | | GAUUUUUU |
| SCN1A- | 16686 | 16686 | 671 | TAATTCAAATA | 1031 | UAAUUCAAAU |
| IVS23 + 646 | 3076 | 3094 | | AGTGGAT | | AAGUGGAU |
| SCN1A- | 16686 | 16686 | 672 | CTGCATAATTC | 1032 | CUGCAUAAUUC |
| IVS23 + 651 | 3071 | 3089 | | AAATAAG | | AAAUAAG |
| SCN1A- | 16686 | 16686 | 673 | CTATTCTGCATA | 1033 | CUAUUCUGCAU |
| IVS23 + 656 | 3066 | 3084 | | ATTCAA | | AAUUCAA |
| SCN1A- | 16686 | 16686 | 674 | GTATTCTATTCT | 1034 | GUAUUCUAUUC |
| IVS23 + 661 | 3061 | 3079 | | GCATAA | | UGCAUAA |
| SCN1A- | 16686 | 16686 | 675 | GGTATGTATTCT | 1035 | GGUAUGUAUU |
| IVS23 + 666 | 3056 | 3074 | | ATTCTG | | CUAUUCUG |
| SCN1A- | 16686 | 16686 | 676 | TTCTAGGTATGT | 1036 | UUCUAGGUAU |
| IVS23 + 671 | 3051 | 3069 | | ATTCTA | | GUAUUCUA |
| SCN1A- | 16686 | 16686 | 677 | TTTATTTCTAGG | 1037 | UUUAUUUCUA |
| IVS23 + 676 | 3046 | 3064 | | TATGTA | | GGUAUGUA |
| SCN1A- | 16686 | 16686 | 678 | TTTGTTTTATTT | 1038 | UUUGUUUUAU |
| IVS23 + 681 | 3041 | 3059 | | CTAGGT | | UUCUAGGU |
| SCN1A- | 16686 | 16686 | 679 | ACGTTTTTGTT | 1039 | ACGUUUUUGU |
| IVS23 + 686 | 3036 | 3054 | | TTATTTC | | UUUAUUUC |
| SCN1A- | 16686 | 16686 | 680 | ATAAGACGTTT | 1040 | AUAAGACGUU |
| IVS23 + 691 | 3031 | 3049 | | TTGTTTT | | UUUGUUUU |
| SCN1A- | 16686 | 16686 | 681 | TCATGATAAGA | 1041 | UCAUGAUAAG |
| IVS23 + 696 | 3026 | 3044 | | CGTTTTT | | ACGUUUUU |
| SCN1A- | 16686 | 16686 | 682 | AATACTCATGA | 1042 | AAUACUCAUGA |
| IVS23 + 701 | 3021 | 3039 | | TAAGACG | | UAAGACG |
| SCN1A- | 16686 | 16686 | 683 | ATCTTAATACTC | 1043 | AUCUUAAUACU |
| IVS23 + 706 | 3016 | 3034 | | ATGATA | | CAUGAUA |
| SCN1A- | 16686 | 16686 | 684 | ATTTTATCTTAA | 1044 | AUUUUAUCUU |
| IVS23 + 711 | 3011 | 3029 | | TACTCA | | AAUACUCA |
| SCN1A- | 16686 | 16686 | 685 | CTTAAATTTTAT | 1045 | CUUAAAUUUU |
| IVS23 + 716 | 3006 | 3024 | | CTTAAT | | AUCUUAAU |
| SCN1A- | 16686 | 16686 | 686 | TATGCCTTAAAT | 1046 | UAUGCCUUAAA |
| IVS23 + 721 | 3001 | 3019 | | TTTATC | | UUUUAUC |
| SCN1A- | 16686 | 16686 | 687 | GAGTTTATGCC | 1047 | GAGUUUAUGCC |
| IVS23 + 726 | 2996 | 3014 | | TTAAATT | | UUAAAUU |
| SCN1A- | 16686 | 16686 | 688 | GAAGTGAGTTT | 1048 | GAAGUGAGUU |
| IVS23 + 731 | 2991 | 3009 | | ATGCCTT | | UAUGCCUU |
| SCN1A- | 16686 | 16686 | 689 | TCTAAGAAGTG | 1049 | UCUAAGAAGU |
| IVS23 + 736 | 2986 | 3004 | | AGTTTAT | | GAGUUUAU |
| SCN1A- | 16686 | 16686 | 690 | CTTATTCTAAG | 1050 | CUUAUUCUAAG |
| IVS23 + 741 | 2981 | 2999 | | AAGTGAG | | AAGUGAG |
| SCN1A- | 16686 | 16686 | 691 | AGTTACTTATTC | 1051 | AGUUACUUAU |
| IVS23 + 746 | 2976 | 2994 | | TAAGAA | | UCUAAGAA |
| SCN1A- | 16686 | 16686 | 692 | TTGGGAGTTAC | 1052 | UUGGGAGUUA |
| IVS23 + 751 | 2971 | 2989 | | TTATTCT | | CUUAUUCU |
| SCN1A- | 16686 | 16686 | 693 | GTTAGTTGGGA | 1053 | GUUAGUUGGG |
| IVS23 + 756 | 2966 | 2984 | | GTTACTT | | AGUUACUU |
| SCN1A- | 16686 | 16686 | 694 | AGAAAGTTAGT | 1054 | AGAAAGUUAG |
| IVS23 + 761 | 2961 | 2979 | | TGGGAGT | | UUGGGAGU |
| SCN1A- | 16686 | 16686 | 695 | ATCCTAGAAAG | 1055 | AUCCUAGAAAG |
| IVS23 + 766 | 2956 | 2974 | | TTAGTTG | | UUAGUUG |
| SCN1A- | 16686 | 16686 | 696 | TTAAAATCCTA | 1056 | UUAAAAUCCUA |
| IVS23 + 771 | 2951 | 2969 | | GAAAGTT | | GAAAGUU |
| SCN1A- | 16686 | 16686 | 697 | ATGTTTTAAAA | 1057 | AUGUUUUAAA |
| IVS23 + 776 | 2946 | 2964 | | TCCTAGA | | AUCCUAGA |
| SCN1A- | 16686 | 16686 | 698 | GTGTTATGTTTT | 1058 | GUGUUAUGUU |
| IVS23 + 781 | 2941 | 2959 | | AAAATC | | UUAAAAUC |
| SCN1A- | 16686 | 16686 | 699 | TCACTGTGTTA | 1059 | UCACUGUGUUA |
| IVS23 + 786 | 2936 | 2954 | | TGTTTTA | | UGUUUUA |
| SCN1A- | 16686 | 16686 | 700 | TGTTTTCACTG | 1060 | UGUUUUCACUG |
| IVS23 + 791 | 2931 | 2949 | | TGTTATG | | UGUUAUG |
| SCN1A- | 16686 | 16686 | 701 | ATGTATGTTTTC | 1061 | AUGUAUGUUU |
| IVS23 + 796 | 2926 | 2944 | | ACTGTG | | UCACUGUG |
| SCN1A- | 16686 | 16686 | 702 | TGTTTATGTATG | 1062 | UGUUUAUGUA |
| IVS23 + 801 | 2921 | 2939 | | TTTTCA | | UGUUUUCA |
| SCN1A- | 16686 | 16686 | 703 | AGTTATGTTTAT | 1063 | AGUUAUGUUU |
| IVS23 + 806 | 2916 | 2934 | | GTATGT | | AUGUAUGU |
| SCN1A- | 16686 | 16686 | 704 | TGTAGAGTTAT | 1064 | UGUAGAGUUA |
| IVS23 + 811 | 2911 | 2929 | | GTTTATG | | UGUUUAUG |
| SCN1A- | 16686 | 16686 | 705 | TAAAATGTAGA | 1065 | UAAAAUGUAG |
| IVS23 + 816 | 2906 | 2924 | | GTTATGT | | AGUUAUGU |
| SCN1A- | 16686 | 16686 | 706 | ATAAATAAAAT | 1066 | AUAAAUAAAA |
| IVS23 + 821 | 2901 | 2919 | | GTAGAGT | | UGUAGAGU |
| SCN1A- | 16686 | 16686 | 707 | TAAGAATAAAT | 1067 | UAAGAAUAAA |
| IVS23 + 826 | 2896 | 2914 | | AAAATGT | | UAAAAUGU |
| SCN1A- | 16686 | 16686 | 708 | AACTTTAAGAA | 1068 | AACUUUAAGA |
| IVS23 + 831 | 2891 | 2909 | | TAAATAA | | AUAAAUAA |
| SCN1A- | 16686 | 16686 | 709 | ACTTAAACTTT | 1069 | ACUUAAACUUU |
| IVS23 + 836 | 2886 | 2904 | | AAGAATA | | AAGAAUA |
| SCN1A- | 16686 | 16686 | 710 | AATACACTTAA | 1070 | AAUACACUUAA |
| IVS23 + 841 | 2881 | 2899 | | ACTTTAA | | ACUUUAA |
| SCN1A- | 16686 | 16686 | 711 | TGTATAATACAC | 1071 | UGUAUAAUAC |
| IVS23 + 846 | 2876 | 2894 | | TTAAAC | | ACUUAAAC |
| SCN1A- | 16686 | 16686 | 712 | CTTCTTGTATAA | 1072 | CUUCUUGUAUA |
| IVS23 + 851 | 2871 | 2889 | | TACACT | | AUACACU |
| SCN1A- | 16686 | 16686 | 713 | CTCTTCTTCTTG | 1073 | CUCUUCUUCUU |
| IVS23 + 856 | 2866 | 2884 | | TATAAT | | GUAUAAU |
| SCN1A- | 16686 | 16686 | 714 | ATAAACTCTTC | 1074 | AUAAACUCUUC |
| IVS23 + 861 | 2861 | 2879 | | TTCTTGT | | UUCUUGU |
| SCN1A- | 16686 | 16686 | 715 | CGAATATAAAC | 1075 | CGAAUAUAAAC |
| IVS23 + 866 | 2856 | 2874 | | TCTTCTT | | UCUUCUU |
| SCN1A- | 16686 | 16686 | 716 | TCTCTCGAATAT | 1076 | UCUCUCGAAUA |
| IVS23 + 871 | 2851 | 2869 | | AAACTC | | UAAACUC |
| SCN1A- | 16686 | 16686 | 717 | TTCTGTCTCTC | 1077 | UUCUGUCUCUC |
| IVS23 + 876 | 2846 | 2864 | | GAATATA | | GAAUAUA |
| SCN1A- | 16686 | 16686 | 718 | ACTTTTTCTGT | 1078 | ACUUUUUCUGU |
| IVS23 + 881 | 2841 | 2859 | | CTCTCGA | | CUCUCGA |
| SCN1A- | 16686 | 16686 | 719 | TTCTGACTTTTT | 1079 | UUCUGACUUUU |
| IVS23 + 886 | 2836 | 2854 | | CTGTCT | | UCUGUCU |
| SCN1A- | 16686 | 16686 | 720 | AAAAATTCTGA | 1080 | AAAAAUUCUG |
| IVS23 + 891 | 2831 | 2849 | | CTTTTTC | | ACUUUUUC |
| SCN1A- | 16686 | 16686 | 721 | CAAACAAAAAT | 1081 | CAAACAAAAAU |
| IVS23 + 896 | 2826 | 2844 | | TCTGACT | | UCUGACU |
| SCN1A- | 16686 | 16686 | 722 | TGATCCAAACA | 1082 | UGAUCCAAACA |
| IVS23 + 901 | 2821 | 2839 | | AAAATTC | | AAAAUUC |
| SCN1A- | 16686 | 16686 | 723 | ATTGGTGATCC | 1083 | AUUGGUGAUCC |
| IVS23 + 906 | 2816 | 2834 | | AAACAAA | | AAACAAA |
| SCN1A- | 16686 | 16686 | 724 | GATATATTGGTG | 1084 | GAUAUAUUGG |
| IVS23 + 911 | 2811 | 2829 | | ATCCAA | | UGAUCCAA |
| SCN1A- | 16686 | 16686 | 725 | GCTATGATATAT | 1085 | GCUAUGAUAU |
| IVS23 + 916 | 2806 | 2824 | | TGGTGA | | AUUGGUGA |
| SCN1A- | 16686 | 16686 | 726 | TGTAAGCTATG | 1086 | UGUAAGCUAU |
| IVS23 + 921 | 2801 | 2819 | | ATATATT | | GAUAUAUU |
| SCN1A- | 16686 | 16686 | 727 | TTTTTTGTAAG | 1087 | UUUUUUGUAA |
| IVS23 + 926 | 2796 | 2814 | | CTATGAT | | GCUAUGAU |
| SCN1A- | 16686 | 16686 | 728 | ACAGTTTTTTT | 1088 | ACAGUUUUUU |
| IVS23 + 931 | 2791 | 2809 | | GTAAGCT | | UGUAAGCU |
| SCN1A- | 16686 | 16686 | 729 | TTAAGACAGTT | 1089 | UUAAGACAGU |
| IVS23 + 936 | 2786 | 2804 | | TTTTTGT | | UUUUUUGU |
| SCN1A- | 16686 | 16686 | 730 | TTTAATTAAGA | 1090 | UUUAAUUAAG |
| IVS23 + 941 | 2781 | 2799 | | CAGTTTT | | ACAGUUUU |
| SCN1A- | 16686 | 16686 | 731 | TGGGTTTTAAT | 1091 | UGGGUUUUAA |
| IVS23 + 946 | 2776 | 2794 | | TAAGACA | | UUAAGACA |
| SCN1A- | 16686 | 16686 | 732 | TGTTGTGGGTT | 1092 | UGUUGUGGGU |
| IVS23 + 951 | 2771 | 2789 | | TTAATTA | | UUUAAUUA |
| SCN1A- | 16686 | 16686 | 733 | AATTATGTTGT | 1093 | AAUUAUGUUG |
| IVS23 + 956 | 2766 | 2784 | | GGGTTTT | | UGGGUUUU |
| SCN1A- | 16686 | 16686 | 734 | AAAAAAATTAT | 1094 | AAAAAAAUUA |
| IVS23 + 961 | 2761 | 2779 | | GTTGTGG | | UGUUGUGG |
| SCN1A- | 16686 | 16686 | 735 | AATCTAAAAAA | 1095 | AAUCUAAAAA |
| IVS23 + 966 | 2756 | 2774 | | ATTATGT | | AAUUAUGU |
| SCN1A- | 16686 | 16686 | 736 | TTAAAAATCTA | 1096 | UUAAAAAUCU |
| IVS23 + 971 | 2751 | 2769 | | AAAAAAT | | AAAAAAAU |
| SCN1A- | 16686 | 16686 | 737 | CTTTCTTAAAA | 1097 | CUUUCUUAAAA |
| IVS23 + 976 | 2746 | 2764 | | ATCTAAA | | AUCUAAA |
| SCN1A- | 16686 | 16686 | 738 | AGAATCTTTCT | 1098 | AGAAUCUUUCU |
| IVS23 + 981 | 2741 | 2759 | | TAAAAAT | | UAAAAAU |
| SCN1A- | 16686 | 16686 | 739 | ATAATAGAATCT | 1099 | AUAAUAGAAU |
| IVS23 + 986 | 2736 | 2754 | | TTCTTA | | CUUUCUUA |

TABLE 8A
Exemplary ASOs to correct intron retention.

| SEQ | | | Re- |
|---|---|---|---|
| ID | | | tained |
| NO: | Name | Sequence (5′-3′) | Intron |
| 115 | SCN1A-IVS21 + 6 | CAGAGAAAAUAGUGUUCA | 21 |
| 116 | SCN1A-IVS21 + 11 | AUAUUCAGAGAAAAUAGU | 21 |
| 117 | SCN1A-IVS21 + 16 | UAAAAAUAUUCAGAGAAA | 21 |
| 118 | SCN1A-IVS21 + 21 | AACAAUAAAAAUAUUCAG | 21 |
| 119 | SCN1A-IVS21 + 26 | UUCCAAACAAUAAAAAUA | 21 |
| 120 | SCN1A-IVS21 + 31 | UAUUAUUCCAAACAAUAA | 21 |
| 121 | SCN1A-IVS21 + 36 | UUUGUUAUUAUUCCAAAC | 21 |
| 122 | SCN1A-IVS21 + 41 | AUUAUUUUGUUAUUAUUC | 21 |
| 123 | SCN1A-IVS21 + 46 | AUGUCAUUAUUUUGUUAU | 21 |
| 124 | SCN1A-IVS21 + 51 | GAUGUAUGUCAUUAUUUU | 21 |
| 125 | SCN1A-IVS21 + 56 | UAAUAGAUGUAUGUCAUU | 21 |
| 126 | SCN1A-IVS21 + 61 | CUAAAUAAUAGAUGUAUG | 21 |
| 127 | SCN1A-IVS21 + 66 | AGGAACUAAAUAAUAGAU | 21 |
| 128 | SCN1A-IVS21 + 71 | UUCUUAGGAACUAAAUAA | 21 |
| 129 | SCN1A-IVS21 + 76 | ACUUUUUCUUAGGAACUA | 21 |
| 130 | SCN1A-IVS21 + 81 | UAUAUACUUUUUCUUAGG | 21 |
| 131 | SCN1A-IVS21 − 16 | UGCAUGUUUUACUUUGGA | 21 |
| 132 | SCN1A-IVS21 − 21 | GUUUUACUUUGGAGUAAA | 21 |
| 133 | SCN1A-IVS21 − 26 | ACUUUGGAGUAAAAAUAA | 21 |
| 134 | SCN1A-IVS21 − 31 | GGAGUAAAAAUAAUUUAG | 21 |
| 135 | SCN1A-IVS21 − 36 | AAAAAUAAUUUAGACCUG | 21 |
| 136 | SCN1A-IVS21 − 41 | UAAUUUAGACCUGAUGUU | 21 |
| 137 | SCN1A-IVS21 − 46 | UAGACCUGAUGUUUAAUA | 21 |
| 138 | SCN1A-IVS21 − 51 | CUGAUGUUUAAUAAAUAU | 21 |
| 139 | SCN1A-IVS21 − 56 | GUUUAAUAAAUAUUCUUA | 21 |
| 140 | SCN1A-IVS21 − 61 | AUAAAUAUUCUUACUGAU | 21 |
| 141 | SCN1A-IVS21 − 66 | UAUUCUUACUGAUAUAAU | 21 |
| 142 | SCN1A-IVS21 − 71 | UUACUGAUAUAAUUUUCA | 21 |
| 143 | SCN1A-IVS21 − 76 | GAUAUAAUUUUCAAAAGG | 21 |
| 144 | SCN1A-IVS21 − 81 | AAUUUUCAAAAGGGAAUA | 21 |
| 145 | SCN1A-IVS21 − 27 | CUUUGGAGUAAAAAUAAU | 21 |
| 146 | SCN1A-IVS21 − 28 | UUUGGAGUAAAAAUAAUU | 21 |
| 148 | SCN1A-IVS21 − 29 | UUGGAGUAAAAAUAAUUU | 21 |
| 149 | SCN1A-IVS21 − 30 | UGGAGUAAAAAUAAUUUA | 21 |
| 150 | SCN1A-IVS21 − 32 | GAGUAAAAAUAAUUUAGA | 21 |
| 151 | SCN1A-IVS21 − 33 | AGUAAAAAUAAUUUAGAC | 21 |
| 152 | SCN1A-IVS21 − 34 | GUAAAAAUAAUUUAGACC | 21 |
| 153 | SCN1A-IVS21 − 35 | UAAAAAUAAUUUAGACCU | 21 |
| 154 | SCN1A-IVS21 − 72 | UACUGAUAUAAUUUUCAA | 21 |
| 155 | SCN1A-IVS21 − 73 | ACUGAUAUAAUUUUCAAA | 21 |
| 156 | SCN1A-IVS21 − 74 | CUGAUAUAAUUUUCAAAA | 21 |
| 157 | SCN1A-IVS21 − 75 | UGAUAUAAUUUUCAAAAG | 21 |
| 158 | SCN1A-IVS21 − 77 | AUAUAAUUUUCAAAAGGG | 21 |
| 159 | SCN1A-IVS21 − 78 | UAUAAUUUUCAAAAGGGA | 21 |
| 160 | SCN1A-IVS21 − 79 | AUAAUUUUCAAAAGGGAA | 21 |
| 161 | SCN1A-IVS21 − 80 | UAAUUUUCAAAAGGGAAU | 21 |
| 162 | | CAAGGAUUAAAGGUAGCA | 21 |

TABLE 8B
Exemplary ASOs to correct intron retention.

| SEQ | | | Re- |
|---|---|---|---|
| ID | | | tained |
| NO: | Name | Sequence (5′-3′) | Intron |
| 163 | SCN1A-IVS21 + 6 | CAGAGAAAATAGTGTTCA | 21 |
| 164 | SCN1A-IVS21 + 11 | ATATTCAGAGAAAATAGT | 21 |
| 165 | SCN1A-IVS21 + 16 | TAAAAATATTCAGAGAAA | 21 |
| 166 | SCN1A-IVS21 + 21 | AACAATAAAAATATTCAG | 21 |
| 167 | SCN1A-IVS21 + 26 | TTCCAAACAATAAAAATA | 21 |
| 168 | SCN1A-IVS21 + 31 | TATTATTCCAAACAATAA | 21 |
| 169 | SCN1A-IVS21 + 36 | TTTGTTATTATTCCAAAC | 21 |
| 170 | SCN1A-IVS21 + 41 | ATTATTTTGTTATTATTC | 21 |
| 171 | SCN1A-IVS21 + 46 | ATGTCATTATTTTGTTAT | 21 |
| 172 | SCN1A-IVS21 + 51 | GATGTATGTCATTATTTT | 21 |
| 173 | SCN1A-IVS21 + 56 | TAATAGATGTATGTCATT | 21 |
| 174 | SCN1A-IVS21 + 61 | CTAAATAATAGATGTATG | 21 |
| 175 | SCN1A-IVS21 + 66 | AGGAACTAAATAATAGAT | 21 |
| 176 | SCN1A-IVS21 + 71 | TTCTTAGGAACTAAATAA | 21 |
| 177 | SCN1A-IVS21 + 76 | ACTTTTTCTTAGGAACTA | 21 |
| 178 | SCN1A-IVS21 + 81 | TATATACTTTTTCTTAGG | 21 |
| 179 | SCN1A-IVS21 − 16 | TGCATGTTTTACTTTGGA | 21 |
| 180 | SCN1A-IVS21 − 21 | GTTTTACTTTGGAGTAAA | 21 |
| 181 | SCN1A-IVS21 − 26 | ACTTTGGAGTAAAAATAA | 21 |
| 182 | SCN1A-IVS21 − 31 | GGAGTAAAAATAATTTAG | 21 |
| 183 | SCN1A-IVS21 − 36 | AAAAATAATTTAGACCTG | 21 |
| 184 | SCN1A-IVS21 − 41 | TAATTTAGACCTGATGTT | 21 |
| 185 | SCN1A-IVS21 − 46 | TAGACCTGATGTTTAATA | 21 |
| 186 | SCN1A-IVS21 − 51 | CTGATGTTTAATAAATAT | 21 |
| 187 | SCN1A-IVS21 − 56 | GTTTAATAAATATTCTTA | 21 |
| 188 | SCN1A-IVS21 − 61 | ATAAATATTCTTACTGAT | 21 |
| 189 | SCN1A-IVS21 − 66 | TATTCTTACTGATATAAT | 21 |
| 190 | SCN1A-IVS21 − 71 | TTACTGATATAATTTTCA | 21 |
| 191 | SCN1A-IVS21 − 76 | GATATAATTTTCAAAAGG | 21 |
| 192 | SCN1A-IVS21 − 81 | AATTTTCAAAAGGGAATA | 21 |
| 193 | SCN1A-IVS21 − 27 | CTTTGGAGTAAAAATAAT | 21 |
| 194 | SCN1A-IVS21 − 28 | TTTGGAGTAAAAATAATT | 21 |
| 195 | SCN1A-IVS21 − 29 | TTGGAGTAAAAATAATTT | 21 |
| 196 | SCN1A-IVS21 − 30 | TGGAGTAAAAATAATTTA | 21 |
| 197 | SCN1A-IVS21 − 32 | GAGTAAAAATAATTTAGA | 21 |
| 198 | SCN1A-IVS21 − 33 | AGTAAAAATAATTTAGAC | 21 |
| 199 | SCN1A-IVS21 − 34 | GTAAAAATAATTTAGACC | 21 |
| 200 | SCN1A-IVS21 − 35 | TAAAAATAATTTAGACCT | 21 |
| 201 | SCN1A-IVS21 − 72 | TACTGATATAATTTTCAA | 21 |
| 202 | SCN1A-IVS21 − 73 | ACTGATATAATTTTCAAA | 21 |
| 203 | SCN1A-IVS21 − 74 | CTGATATAATTTTCAAAA | 21 |
| 204 | SCN1A-IVS21 − 75 | TGATATAATTTTCAAAAG | 21 |
| 205 | SCN1A-IVS21 − 77 | ATATAATTTTCAAAAGGG | 21 |
| 206 | SCN1A-IVS21 − 78 | TATAATTTTCAAAAGGGA | 21 |
| 207 | SCN1A-IVS21 − 79 | ATAATTTTCAAAAGGGAA | 21 |
| 208 | SCN1A-IVS21 − 80 | TAATTTTCAAAAGGGAAT | 21 |
| 209 | | CAAGGATTAAAGGTAGCA | 21 |

### Example 15: Alternative Splicing of SCN1A Pre-mRNA that Results in NMD is Conserved in Multiple Species and in a DS Patient

Bioinformatic analysis of human brain samples revealed an exon inclusion event in the SCN1A gene that leads to a frameshift and the introduction of a premature termination codon. This example demonstrates validation of this non-productive splicing event in various species, including in a human DS patient.

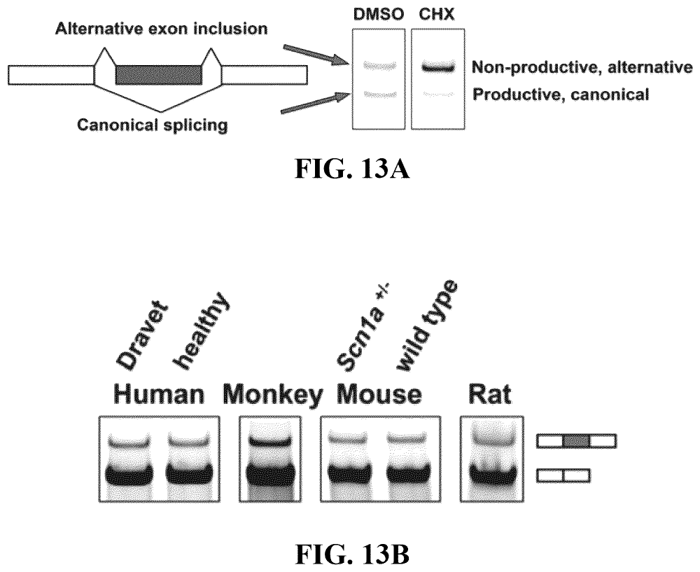

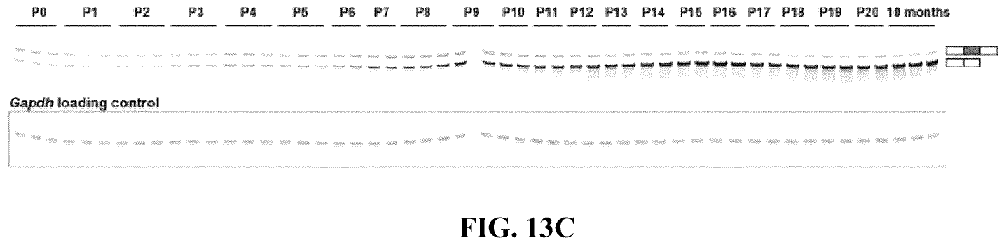

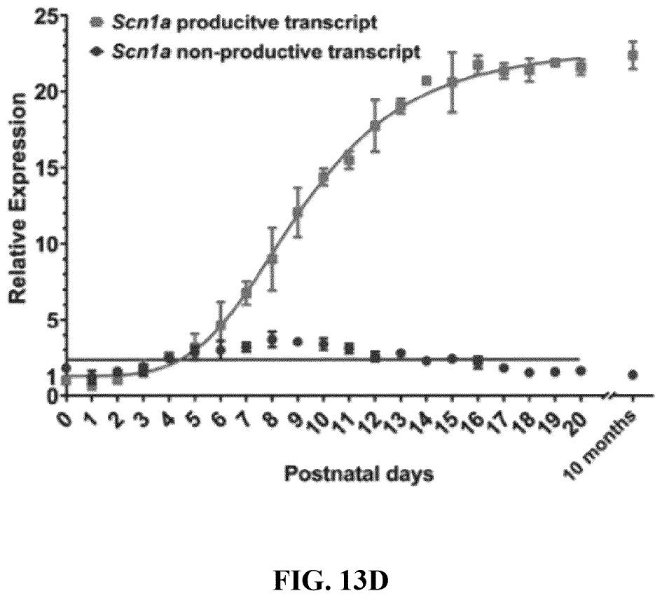

FIG. 13A shows a schematic representation and validation of this non-productive splicing event by RT-PCR of human cells treated with or without the translation inhibitor, cycloheximide (CHX). The productive (canonical) isoform (bottom band) does not contain the alternative exon and translates into functional Na_{V}1.1 protein. The non-productive isoform contains the alternative exon (top band). DMSO: Dimethyl sulfoxide, CHX: Cycloheximide.

BLAST searches of the NCBI database identified highly conserved homologs of this SCN1A NMD-inducing exon in multiple species and the results were verified by RT-PCR (FIG. 13B). Moreover, the presence of the NMD-inducing exon inclusion event is retained in brain tissues from a DS patient (FIG. 13B), suggesting that the level of the NMD-exon inclusion event is unchanged between healthy and DS individuals. FIG. 13B shows TBE-PAGE of the RT-PCR products of SCN1A transcripts in cerebral cortex from a healthy female subject, a DS patient, a cynomolgus monkey, a DS mouse, a WT mouse (C57BL/6J) and a Sprague Dawley rat. Canonical SCN1A transcript is represented by the lower bands and product corresponding to the NMD-exon inclusion is represented by the upper bands. Patient variants located in the NMD-inducing exon region that was identified here have been shown to cause an increase in NMD-exon inclusion levels and a reduction of productive mRNA leading to DS. See Carvill et al., Am J Hum Genet 103, 1022-1029 (2018).

RT-PCR analysis was also performed to measure the inclusion level of the NMD-inducing exon and productive Scn1a mRNA in coronal brain sections from P0-P20 and 10-month-old WT C57BL/6J mice (FIG. 13C). Densitometric measurement of the RT-PCR products showed that the NMD-inducing exon containing transcript (upper band) is largely unchanged during development while the productive mRNA (lower band) increases (FIG. 13D). Productive Scn1a transcript levels (FIG. 13D) begin to increase dramatically around P7-8. As shown in FIG. 13D, expression of Scn1a transcripts was first normalized to endogenous Gapdh and then to the Scn1a productive transcript at P0. Data are presented as mean±SD in the figure (n=2 or 3 samples from individual animals for each data point). Expression of Scn1a productive transcript was fit to a four-parameter non-linear curve. Expression of Scn1a non-productive transcript was best fit to a linear curve. These results suggested that it may be possible to increase total productive transcript levels by converting NMD-exon containing transcripts to productive transcripts and that the impact of this manipulation would be the greatest during early postnatal brain development.

### Example 16: ASO Administration Increases SCN1A Expression in Cultured Human Cells and Mouse Brain

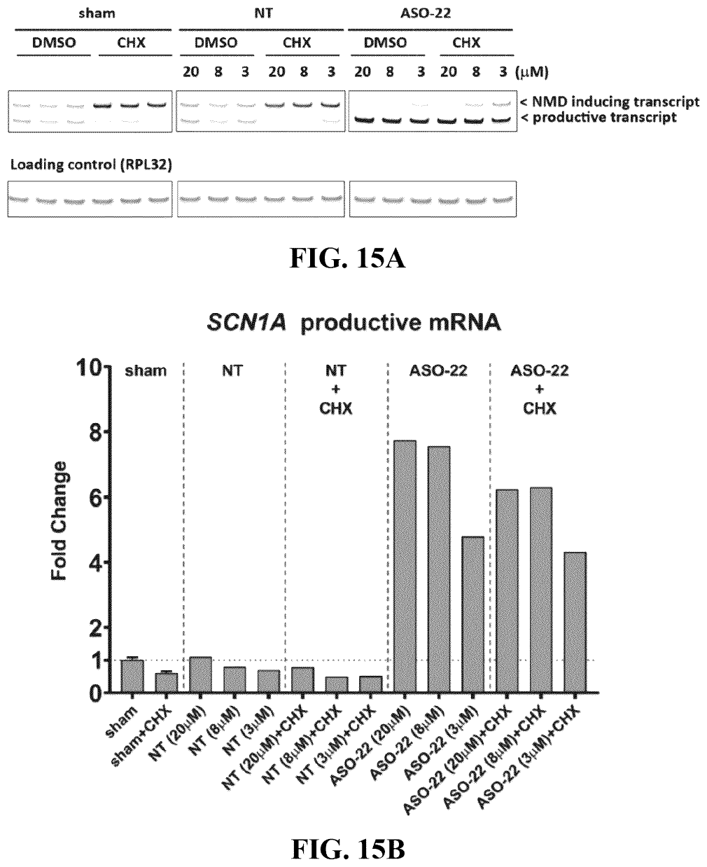

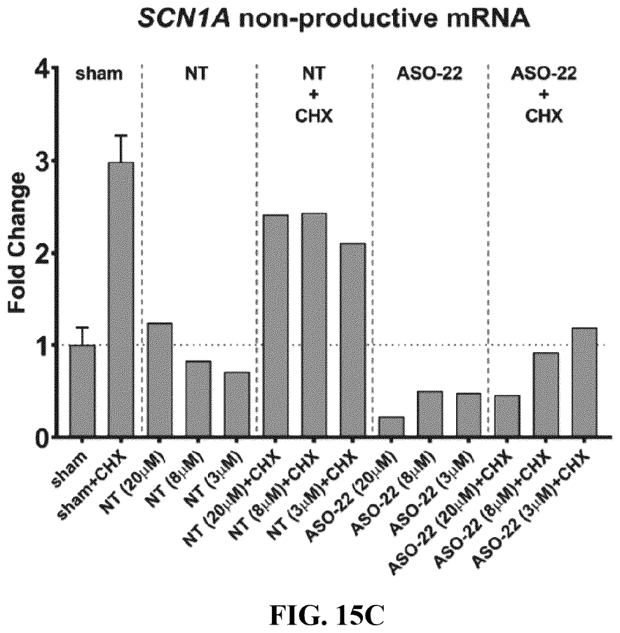

A series of ASOs were designed, which bind to human SCN1A exon 20X and the surrounding intronic sequences. All ASOs used in the initial screening were based on 2′-methoxy-ethyl (MOE) modification of the oligonucleotide with a phosphorothioate backbone. ASOs were screened in human neural progenitor cells by free uptake, followed by analysis of SCN1A productive and exon 20X-containing nonproductive transcripts with RT-PCR and qPCR. As shown in FIG. 14A, a total of 47 ASOs were screened at 20 μM. A non-targeting ASO (NT) and no ASO control (−) were included. RPL32 was used as loading control. A number of ASOs were identified that significantly decreased the NMD-inducing exon inclusion and increased productive transcript expression (FIGS. 14A-14C). The most active ASO (ASO-22, indicated by the arrows in FIGS. 14A-14C) was selected for further evaluation. It was confirmed that the effect of ASO-22 is dose-dependent (FIG. 15). A non-targeting ASO control (NT) had no effect on the level of either transcript isoform (FIG. 15).

Dose-response relationships for ASO-22 were determined in human neural progenitor cells using free uptake or nucleofection delivery methods. The EC50 values were 3 μM, determined by free uptake, and 529 nM, determined by nucleofection (FIG. 14D). The specificity of ASO-22 was also assessed by measuring its effect on the expression of four highly homologous VGSC α subunit genes in treated cells. No change in the expression levels of SCN2A, SCN3A, SCN8A, or SCN9A were observed in ReNcells following 20, 8 or 3 μM ASO treatment via free uptake (FIG. 14E). These results indicate that ASO-22 potently and specifically increases productive SCN1A mRNA in human neural progenitor cells.

FIG. 14A shows ASO screen in ReNcells. TBE PAGE of RT-PCR products corresponding to SCN1A productive (lower bands, 549 bp) and non-productive mRNA containing exon 20X (upper bands, 613 bp) in ReNcells after gymnotic (free) uptake of ASO. FIG. 14B shows percentage of exon 20X inclusion in SCN1A transcript, as quantified from the RT-PCR products shown in FIG. 14A. PCR products were quantified by densitometry and plotted as percentage of the exon 20X-containing mRNA over total (exon 20X-containing and productive mRNA). n=1 for each ASO treatment group; n=2 for NT; n=11 for sham. FIG. 14C shows expression of SCN1A productive mRNA in ASO-treated ReNcells determined by SYBR green qPCR. Expression of SCN1A productive mRNA was first normalized to endogenous RPL32 and then to no ASO control (−). qPCR results were presented as mean±SD for each treatment. n=1 for each ASO treatment; n=2 for NT; n=10 for sham. FIG. 14D demonstrates the dose-response relationship of ASO-22 in ReNcells with free uptake and nucleofection. Expression of SCN1A was normalized to endogenous RPL32 and then to no ASO control. qPCR results were presented as mean±SD (n=2 for both treatments). Expression of SCN1A productive transcript was fit to four-parameter non-linear curves. FIG. 14E demonstrates effect of selected ASOs on expression of homologous VGSC α subunit genes in ReNcells. Expression of SCN2A, 3A, 8A, and 9A in ReNcells was measured by probe-based qPCR, following gymnotic uptake of 20, 8 or 3 μM of ASO-22 or a non-target. ASO control (NT). SON mRNA levels were first normalized to endogenous Gapdh and then to no ASO (−) treated cells. qPCR results were presented as mean±SD for each analysis (n=4 for each ASO treatment; n=8 for no ASO control.

FIGS. 15A-15C shows dose-dependent effects of ASO-22 on splicing and expression of SCN1A mRNA in ReNcells. ReNcells were treated with 20, 8, or 3 μM of ASO-22 for 72 h by gymnotic uptake, followed by DMSO or cycloheximide (CHX) treatment for 3 h before harvesting. A non-targeting ASO control (NT) and no ASO control (sham) were included. FIG. 15A shows TBE PAGE of RT-PCR products corresponding to SCN1A productive mRNA (lower bands, 549 bp) and non-productive mRNA containing exon 20X (upper bands, 613 bp) in ReNcells. RPL32 was used as the loading control. Expression of SCN1A transcript in ReNcells after gymnotic uptake of ASO-22 was quantified by probe-based qPCR. The levels of SCN1A productive (FIG. 15B) and non-productive (FIG. 15C) mRNA were measured separately with two qPCR assays (see description in the Methods). Transcript expression was first normalized to the endogenous RPL32 signal and then to the no ASO (sham) control. Sham qPCR results are presented as mean±SD for each group. (Independent repeats of experiment: n=1 for ASO-22 treatment; n=3 for sham; n=3 for sham+CHX).

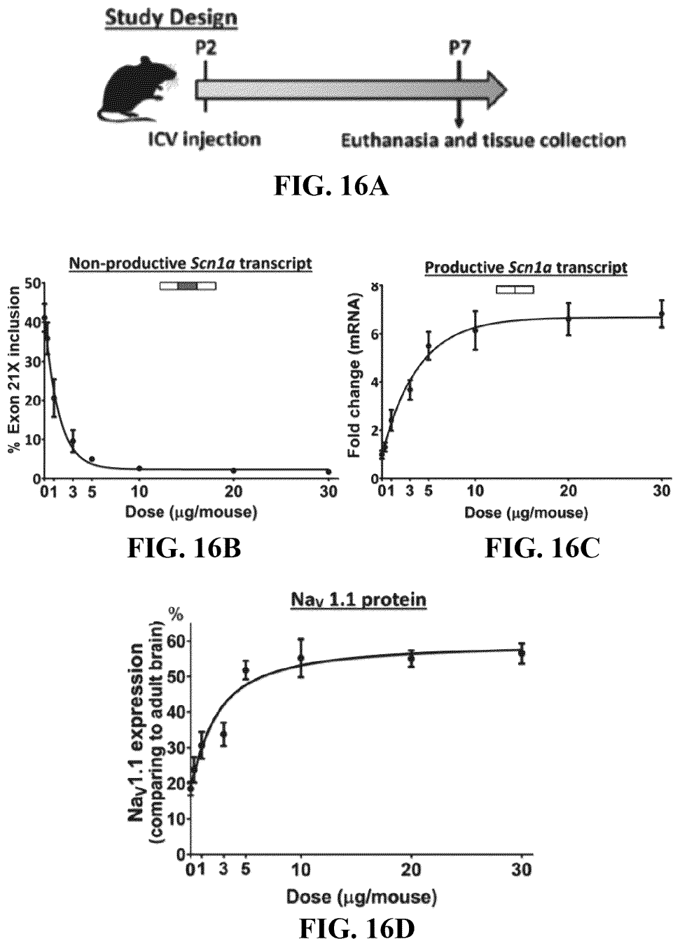

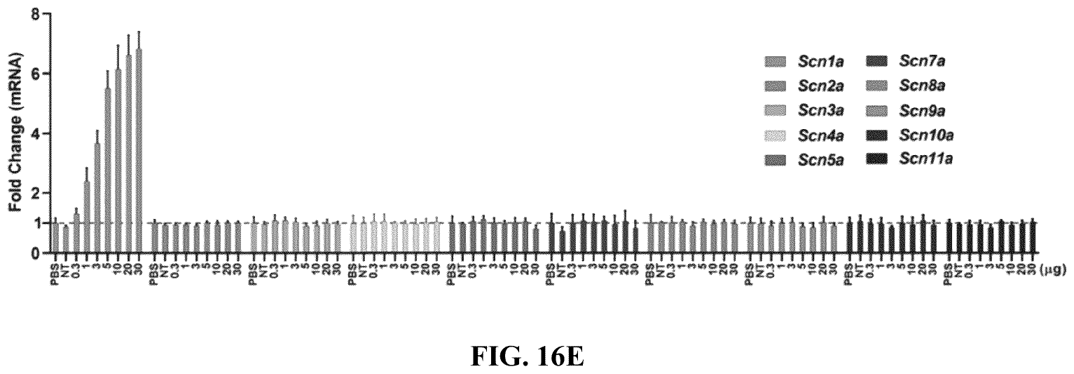

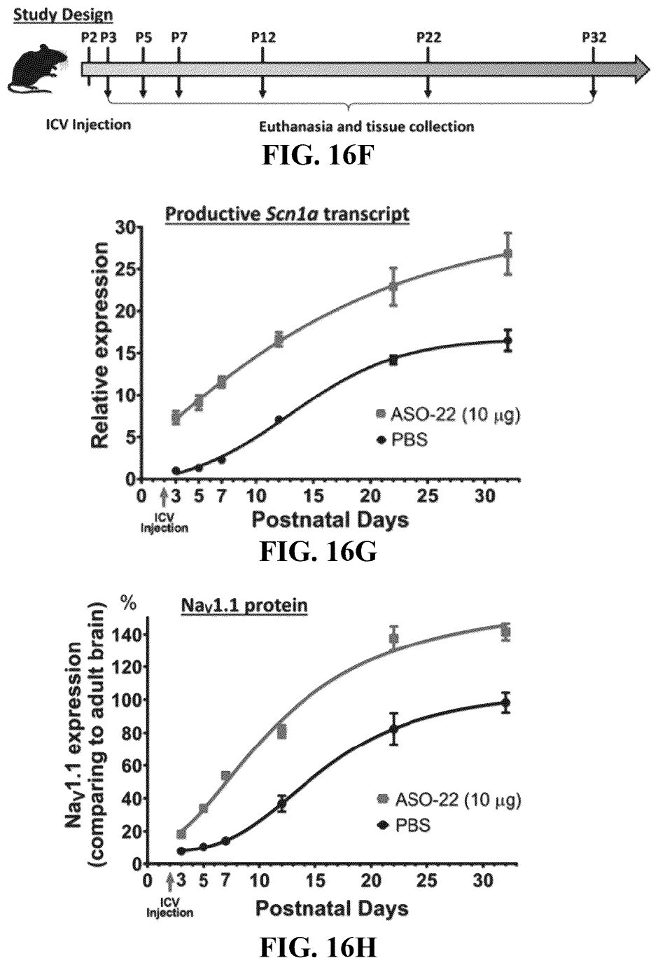

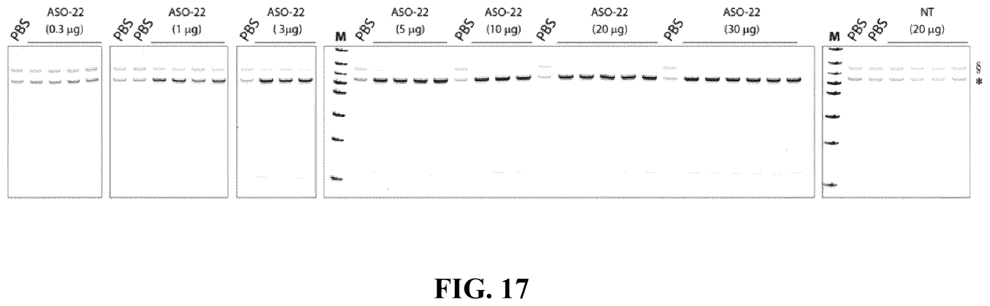

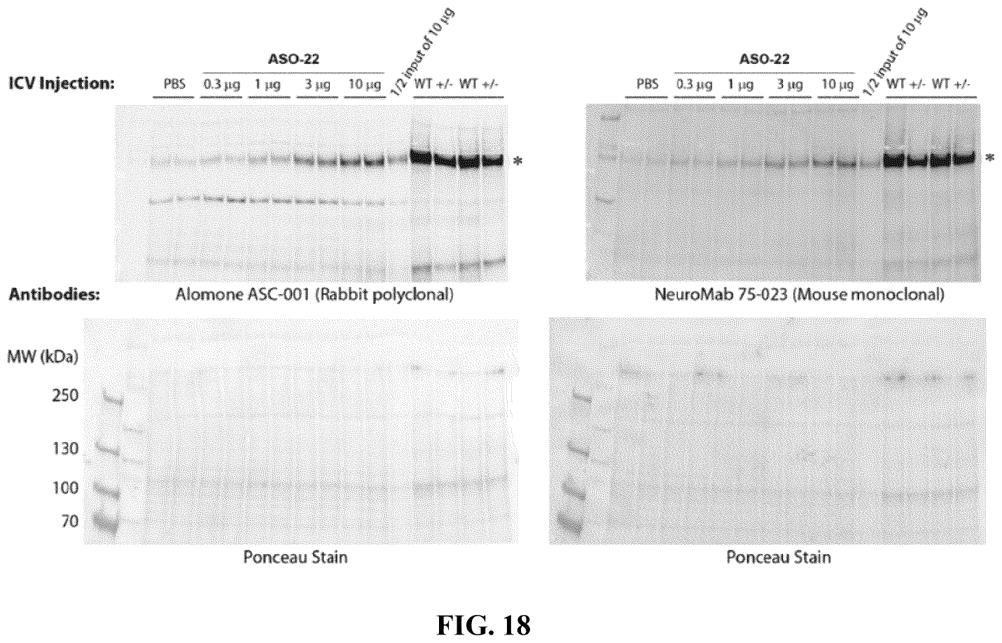

To determine whether ASO-22 administration can upregulate productive mRNA and protein in a dose-dependent manner in vivo, P2 WT C57BL/6J mice were dosed with a single ICV injection of ASO-22 at 0.3, 1, 3, 5, 10, 20 or 30 μg and euthanized 5 days after treatment (FIG. 16A). Brain tissues were collected and analyzed for changes in levels of non-productive and productive Scn1a transcripts, respectively, as well as Na_{V}1.1 protein. Inclusion of the NMD exon in Scn1a transcript decreased with increasing ASO dose (FIG. 16B). A dose-dependent increase in productive Scn1a expression was also observed using probe-based qPCR (FIG. 16C) and RT-PCR (FIG. 17), with a ˜6-fold increase in expression level detected in the 10-μg dose group and no further increase was observed at 20 and 30 μg. Finally, expression levels of Na_{V}1.1 protein increased in a similar dose-dependent manner (FIG. 16D and FIG. 18). A non-targeting ASO control (NT) had no effect on the level of either Scn1a transcript or Na_{V}1.1. The expression of eight closely related VGSC α subunit genes plus Nax (Scn7a) were unaffected in the brains of mice that had received the ASO (FIG. 16E).

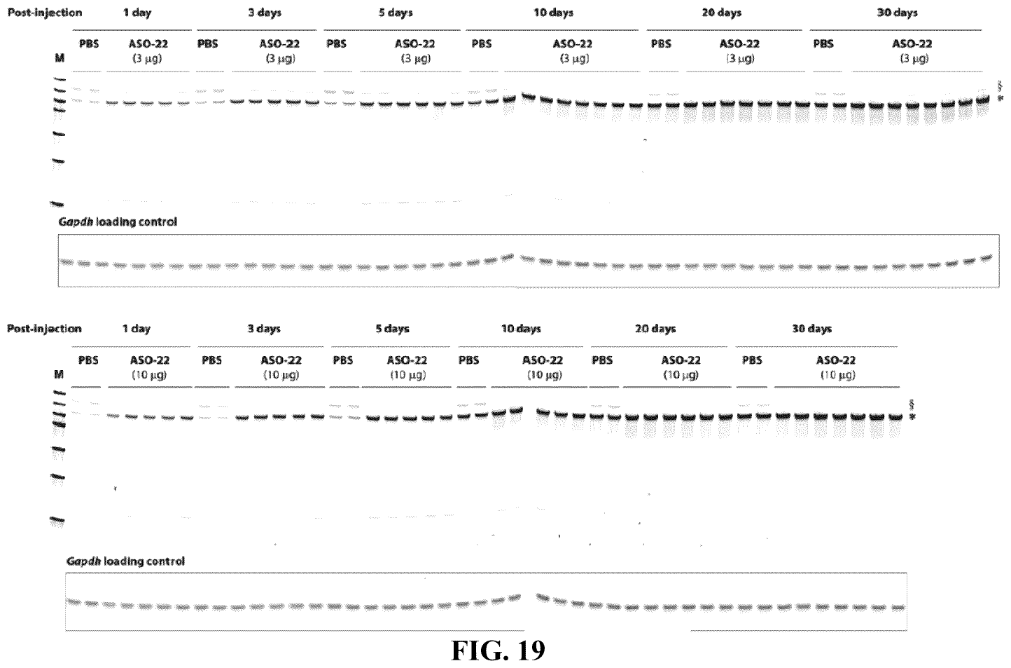

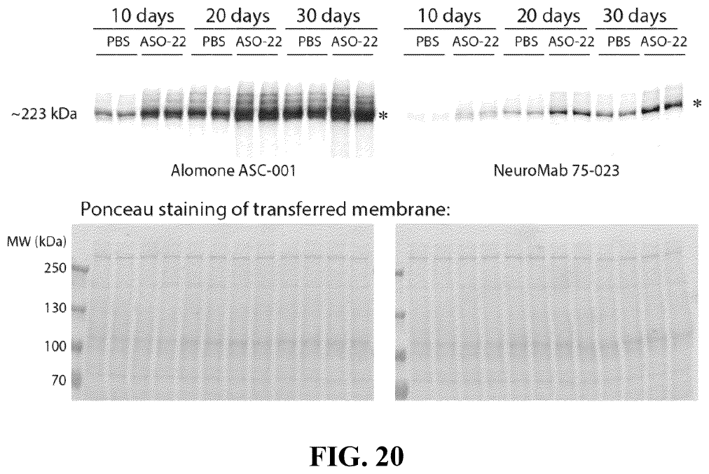

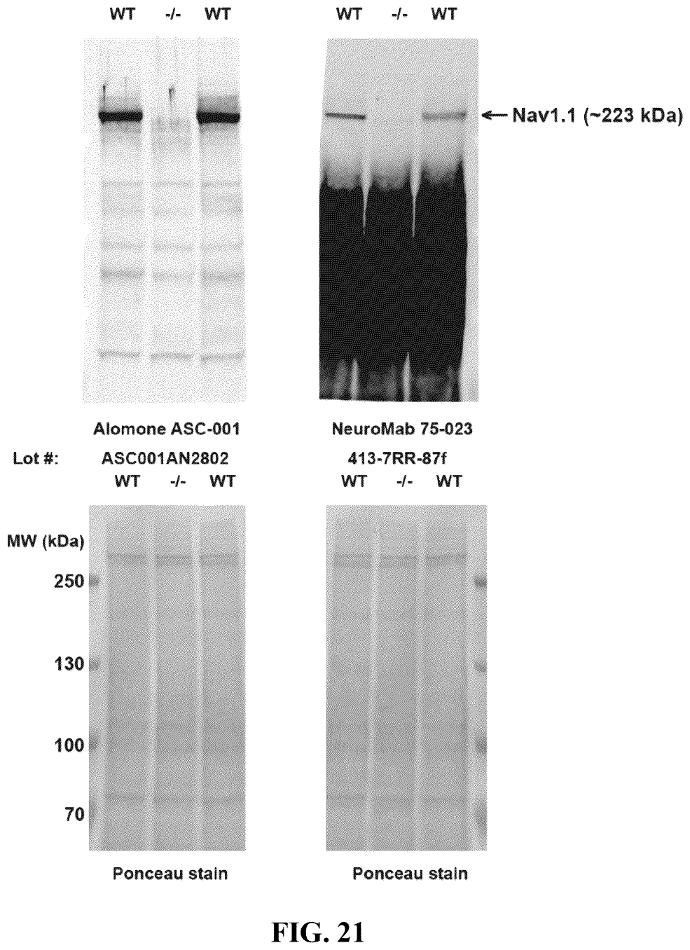

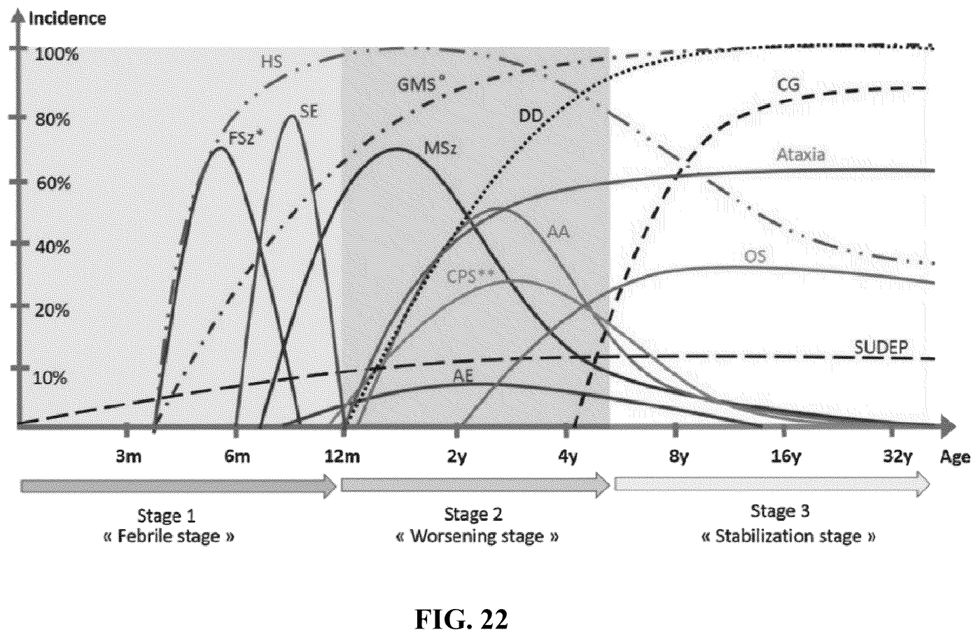

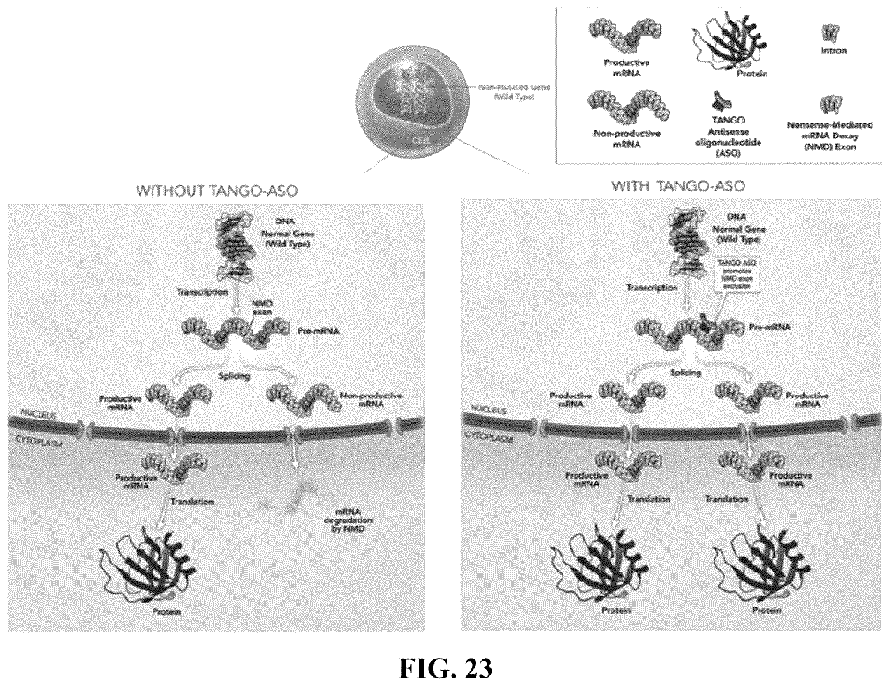

To determine the durability of the ASO-22 effect in brain, P2 WT mice ICV-injected with 10 μg of ASO-22 were examined for brain expression of Scn1a productive transcript levels at varying time points after injection (FIG. 16F). Increased levels of productive Scn1a transcript were observed for up to 30 days post injection (FIG. 16G and FIG. 19). Levels of Na_{V}1.1 protein also increased and were maintained during the 30-day observation period compared to PBS injection controls (FIG. 16H and FIG. 20).

FIG. 16A shows experimental design for ASO-22 dose-response relationship in vivo. FIG. 16B shows percentage of NMD-inducing exon inclusion in Scn1a transcript in mouse brains, as quantified by densitometry of TBE PAGE RT-PCR products (gels shown in FIG. 17). Quantification results are presented as mean±SD (n=3-14 for each treatment group). Plotted data in FIG. 16B were fit to a four-parameter non-linear curve. Similar to PBS treated animals, the percentage of NMD-inducing exon inclusion for NT ASO treatment group was 45.8%±0.7%. FIG. 16C shows fold changes in expression of Scn1a productive transcript in mouse brains, as quantified by probe-based qPCR. qPCR results are presented as mean±SD (n=3-14 for each treatment group). Plotted data were fit to a four-parameter non-linear curve. Similar to PBS treated animals, the fold change in Scn1a mRNA for the NT ASO treatment group was 0.9 ±0.1. FIG. 16D shows fold changes in expression of Na_{V}1.1 protein in mouse, as quantified by the Meso Scale Discovery (MSD) method. Quantification results are presented as mean±SD (n=3-8 for each treatment group). Plotted data were fit to a four-parameter non-linear curve. Similar to PBS treated animals, Na_{V}1.1 expression for the NT ASO treatment group was 16.8%±0.9% compared to adult brain. FIG. 16E shows effect of ASO-22 on expression of the 9 VGSC α subunit genes plus Nax (Scn7a) expressed in mouse brain. Expression of Scn1a productive transcript and the remaining 8 VGSC α subunit genes plus Nax (Scn7a) in mouse brains following ICV injection of PBS, a non-target ASO control (NT, 20 μg) or different doses of ASO-22 was measured by probe-based qPCR. Expression of each transcript was first normalized to endogenous Gapdh then compared to PBS injection controls. qPCR assays used here are listed in the Methods. Data are presented as mean±SD (n=3-14 for each treatment group). FIG. 16F shows experimental design for assessment of duration of effect in mouse brains following ICV injection of ASO-22. FIG. 16G shows quantification of Scn1a productive transcript in mouse brains at selected days post injection of 10 μg of ASO-22 at P2. Scn1a transcript was first normalized to endogenous Gapdh, then compared to mouse brains 1 day after receiving the PBS injection. qPCR results were presented as mean±SD (n=4-9 for each treatment group). Plotted data were fit to a four-parameter non-linear curve. FIG. 16H shows fold changes in expression of Na_{V}1.1 protein in mouse brains at selected days post injection of 10 μg of ASO-22 at P2. Na_{V}1.1 protein expression was quantified by the MSD method. MSD results were presented as mean±SD (n=4-5 randomly selected samples from each treatment group).

FIG. 17 shows dose-dependent effects of ASO-22 on expression of Scn1a in ICV-injected neonatal mouse brains. WT C57BL/6J mice were ICV injected with PBS, a non-target (NT, 20 μg) control, 0.3, 1, 3, 5, 10, 20 or 30 μg of ASO-22 at P2. Brains were harvested 5 days after injection and analyzed for Scn1a mRNA expression. TBE PAGE of RT-PCR products shows Scn1a productive (bottom bands, 498 bp, indicated by *) and NMD-inducing transcript (upper bands, 562 bp, indicated by §) in mouse brains. Quantification of the percentage of exon 21X inclusion is shown in FIG. 16B. Quantification of the Scn1a productive mRNA expression by qPCR is shown in FIG. 16C. M: DNA ladder, NEB, N3231L.

FIG. 18 shows dose-dependent effects of ASO-22 on expression of Na_{V}1.1 in ICV-injected neonatal mouse brains. WT C57BL/6J mice were ICV injected with 0.3, 1, 3 or 10 μg of ASO-22 or PBS at P2. Brains were harvested 5 days after injection and analyzed for Na_{V}1.1 expression. Na_{V}1.1 expression (˜223 kDa, indicated by *) was assessed by immunoblotting of two randomly selected brain samples from each dosing group. 50 μg of protein was loaded per lane. Ponceau S-stained blots are included to show equal loading. Half input of protein from a brain injected with 10 μg of ASO was included for testing signal saturation of the immunoblotting. Brain samples from untreated Scn1a^{31/-} and WT littermate mice were included as controls. Quantification of Na_{V}1.1 expression by Meso Scale Discovery (MSD) method is shown in FIG. 16D.

FIG. 19 shows Expression of Scn1a mRNA in mouse brains at different post-injection days. WT C57BL/6J mice were ICV injected with 3 or 10 μg of ASO-22 or PBS at P2. Brains were harvested 1, 3, 5, 10, 20 or 30 days after injection and analyzed for Scn1a mRNA expression. TBE PAGE of RT-PCR products shows Scn1a productive (bottom bands, 498 bp, indicated by *) and NMD-inducing transcript (upper bands, 562 bp, indicated by §) in mouse brains. Gapdh was used as loading control. Quantification of the Scn1a productive mRNA expression by qPCR is shown in FIG. 16G. M: DNA ladder, NEB, N3231L.

FIG. 20 shows expression of Na_{V}1.1 in mouse brains at different post-injection days. WT C57BL/6J mice were ICV injected with 10 μg of ASO-22 or PBS at P2. Na_{V}1.1 expression (˜223 kDa, indicated by *) was assessed by immunoblotting of two randomly selected brain samples from the 10-, 20-, and 30-day post-injection mice. 50 μg of total protein was loaded per lane. Ponceau S-stained blots are included to show equal loading. Quantification of changes in Na_{V}1.1 expression by Meso Scale Discovery (MSD) method is shown in FIG. 16H.

### Example 17: Treating Dravet Syndrome

Dravet syndrome (DS) is a rare infantile-onset drug-resistant developmental and epileptic encephalopathy with a poor long-term prognosis. Dravet syndrome affects an estimated 1:15,700 individuals in the United States, or a population of approximately 20,000 individuals (Wu et al., 2015, Pediatrics, 136:e1310-5, of which entire content is incorporated herein by reference). Dravet syndrome is most commonly characterized by a pathogenic mutation in the SCN1A gene (Scheffer 2012, Eur. J. Paediatr. Neurol. 16, Suppl 1: S5-8, of which entire content is incorporated herein by reference). Single nucleotide substitutions, small insertions or deletions, and even whole gene deletions have been reported, with at least 1257 different mutations in the SCN1A gene having been described in DS patients to date (Djémié et al., 2016, Mol Genet Genomic Med. 4:457-64, of which entire content is incorporated herein by reference). The SCN1A gene codes for the sodium voltage-gated channel alpha subunit (Na_{V}1.1 protein). SCN1A (Na_{V}1.1 channel) mutations associated with DS are mostly truncating or missense mutations that lead to loss-of-function of the Na_{V}1.1 protein in >95% of cases (Catterall et al., 2010, J. Physiol. 588:1849-59; Meng et al., 2015, Hum Mutat. 2015; 36:573-80, of which entire content is incorporated herein by reference). The mutations are heterozygous in DS patients and result in haploinsufficiency of the Na_{V}1.1 protein.

Several SCN1A mutations (Thr226Met, Val422Leu) have been identified in children with early infantile SCN1A encephalopathy, a profound developmental and epileptic encephalopathy that is phenotypically distinct from DS (Sadleir et al., 2017, Neurology, 89:1035-42, of which entire content is incorporated herein by reference). Upon functional biophysical testing using dynamic action potential clamp assessment, the mutations found to be causative for the early infantile disease have been demonstrated to cause gain-of-function changes in the Na_{V}1.1 protein (Berecki et al., 2019, Ann Neurol. 85:514-25, of which entire content is incorporated herein by reference). Several other mutations have been found using functional testing to cause gain-of-function changes in Na_{V}1.1 (V1611F, D1866Y, W1204R) that are also associated with a seizure phenotype that differs from DS (Meng et al., 2015, Hum Mutat. 2015; 36:573-80, of which entire content is incorporated herein by reference). In addition, Familial Hemiplegic Migraine (FHM), a disease not typically associated with epilepsy, has been shown in some cases to be associated with gain-of-function mutation in SCN1A (L263V, T1174S, Q1489K, F1499L, L1624P, L1649Q, L1670W) (Dhifallah et al., 2018, Front Mol. Neurosci. 11:232; Fan et al., 2016, Cephalalgia. 36:1238-47; Cestele et al., 2013, Proc. Natl. Acad. Sci. USA. 2013; 110:17546-51, of which entire content is incorporated herein by reference). Other potential gain-of-function SCN1A mutations have been associated with non-DS symptoms such as Rasmussen Syndrome (Arg1575Cys) (Depienne et al., 2009, J. Med Genet. 46:183-91, of which entire content is incorporated herein by reference).

Loss of Na_{V}1.1 channels in inhibitory interneurons may cause epilepsy and premature death in patients with DS (Cheah et al., 2012, PNAS. 109:14646-51, of which entire content is incorporated herein by reference). The loss of Na_{V}1.1 channels in other nerve cells likely contributes to the seizures, as well as other aspects of DS (Liu et al., 2013, Ann Neurol. 74:128-39, of which entire content is incorporated herein by reference). Dravet syndrome is characterized by multiple seizure types and frequently progresses to status epilepticus or prolonged seizures lasting more than 5 minutes that require immediate intervention. Almost all patients (>90%) suffer from several comorbidities in addition to seizures including motor and speech impairment, severe intellectual and developmental disabilities, learning difficulties, autism, Attention-Deficit/Hyperactivity Disorder, sleep and gait abnormalities, and behavioral difficulties (Lagae et al., 2018, Dev. Med. Child Neurol. 60:63-72, of which entire content is incorporated herein by reference). As a result, DS patients have a remarkably low quality of life (Lagae et al., 2018, Dev. Med. Child Neurol. 60:63-72, incorporated herein by reference).

Neurologic examination and cognition are usually normal in children with DS up to 2 years of age (Ragona et al., 2011, Epilepsia. 52:386-92, of which entire content is incorporated herein by reference). However, among DS patients >4 years of age, nearly 100% have intellectual impairment (Genton et al., 2011, Epilepsia. 52, Suppl 2:44-9; Ragona et al., 2011, Epilepsia. 52:386-92, of which entire content is incorporated herein by reference). The degree of neurobehavioral impairment ranges from minor learning difficulty to intellectual disability. The time period between 1 year and 8 years of age (the Worsening Stage) is a critical interval for intervention (FIG. 3). After 8 years of age, nearly 100% of DS patients have evidence of substantial intellectual disability (Gataullina and Dulac, 2017, Seizure 44:58-64, of which entire content is incorporated herein by reference). Cognitive impairment in DS is not purely a consequence of seizures. Patients with few seizures may have very severe encephalopathy, and conversely, patients with frequent seizures may have relatively little cognitive decline. In addition, there does not appear to be a correlation between cognitive outcome and SCN1A mutation type, whether a missense or truncating mutation (Ragona et al., 2011, Epilepsia. 52:386-92, incorporated herein by reference). Longer use of contra-indicated medications (e.g., sodium channel blockers) in the first 5 years of disease can have negative effects on cognitive outcome in people with DS (de Lange et al., 2018, Epilepsia, 59:1154-65, of which entire content is incorporated herein by reference).

Dravet syndrome is among the most drug-resistant forms of epilepsy, with more than 90% of patients continuing to have uncontrolled seizures despite treatment with multiple antiepileptic drugs (AEDs), putting them at high risk for injury or death. The primary goal of therapy for DS is to reduce seizure frequency and severity; however, there remains a significant need for additional therapies for these patients that address the other comorbidities of DS. Dravet syndrome is associated with low quality of life (Lagae et al., 2018, Dev. Med. Child. Neurol. 60:63-72, incorporated herein by reference). Reducing seizure frequency and improving cognition and gait will likely have a dramatic improvement in quality of life.

### Therapeutic Agent

An exemplary therapeutic agent for treating Dravet syndrome is an antisense oligonucleotide or antisense oligomer (ASO) medicine that targets ribonucleic acid (RNA) splicing to increase protein levels for the treatment of severe genetic diseases. Specifically, Compound-A is an embodiment of an ASO for the treatment of DS, which is characterized by mutations in the SCN1A gene. Dravet syndrome is most often characterized by mutations in one allele of the SCN1A gene. These patients possess one wild-type allele and one mutant allele. While the Na_{V}1.1 protein is produced from the wild-type allele, the mutant allele is translated into non-functional protein and results in 50% of the normal protein expression in the patient. Compound-A was designed to increase the level of productive SCN1A messenger RNA (mRNA) and consequently increase the expression of the sodium channel Na_{V}1.1 protein. That is, Compound-A binds to the SCN1A pre-mRNA and redirects the splicing machinery to decrease the amount of non-productive mRNA and increases productive mRNA, which is translated into increased Na_{V}1.1 protein from the wild-type allele. Restoring Na_{V}1.1 to physiological levels may reduce both the occurrence of seizures and other non-seizure comorbidities. This RNA-based approach may not be gene therapy, but rather RNA modulation, as it does not manipulate nor insert genetic deoxyribonucleic acid code.

Compound-A was developed to utilize TANGO (Targeted Augmentation of Nuclear Gene Output) technology to leverage a naturally occurring non-productive alternatively spliced exon in human and mouse SCN1A that leads to the incorporation of a premature termination codon and subsequent transcript (mRNA) degradation. These non-productive splicing events are a part of normal gene regulation, and the non-productive splicing events are part of the wild-type or normal sequence of the SCN1A gene. Non-productive splicing events amenable to TANGO are alternative splicing events that lead to nonsense-mediated mRNA decay exons, or NMD exons. NMD exons are found in over 10% of gene transcripts and, like retained introns, are part of the wild-type sequence of the gene. Non-productive mRNA, which includes these NMD exons, is degraded in the cytoplasm of the cell by nonsense-mediated mRNA decay and is not translated into protein. Compound-A binds to the pre-mRNA and redirects the splicing machinery to prevent inclusion of the NMD exon. This splice-switching decreases non-productive mRNA and increases productive mRNA, which is translated into increased full-length functional protein from the wild-type allele. The TANGO mechanism upregulates expression of the wild-type allele, meaning the TANGO mechanism does not rely on targeting a specific mutation. TANGO ASOs decrease the amount of non-productive mRNA and increase the level of productive mRNA, leading to the generation of more protein. TANGO operates in a mutation-independent manner, given it utilizes one wild-type allele, and does not alter protein coding splicing isoforms.

Scn1a mRNA and Na_{V}1.1 protein levels in rodent brains peak at 4 weeks after birth. In mice the rise in Na_{V}1.1 channels begins at 10 days postnatal and continues through 4 weeks of age (Cheah et al., 2013, Channels, 7:468-72, of which entire content is incorporated herein by reference). Human brain immunoblotting with subtype-specific antibodies showed that Na_{V}1.1 protein expression parallels that in rodent brain, with levels of Na_{V}1.1 being low at birth, steadily increasing to peak values by 20 months, and remaining steady through 30 months (Cheah et al., 2013, Channels, 7:468-72, incorporated herein by reference). A separate study in human brain showed Na_{V}1.1 immunoreactive neurons and neurites increased during the late fetal and postnatal periods, reached their peaks 7 to 9 months after birth (Wang et al., 2011, Brain Res. 1389:61-70, of which entire content is incorporated herein by reference). A similar pattern has been observed in non-human primates (NHPs) and humans. Although the absolute amount of NMD substrate is similar in brain tissues obtained from mice of different ages, because of the progressive increase in productive Scn1a mRNA and Na_{V}1.1 protein in the brain with age, the fold change in Scn1a and Na_{V}1.1 in Compound-A-treated mice is higher in younger animals than in older animals. Therefore, Compound-A treatment may be more effective in restoring Na_{V}1.1 protein back to physiologically normal levels in pediatric DS patients. Preclinical testing in a neonatal mouse model of DS using Compound-A showed significant mortality benefit in animals 35 to 90 days postnatally (equivalent to a greater than 2-year-old human) (Arzimanoglou et al., 2018, Pediatr. Drugs. 20:249-64, of which entire content is incorporated herein by reference).

Oligonucleotide-based compounds are developed for the treatment of human brain disorders by direct delivery inside the blood-brain barrier. Oligonucleotides dosed directly in the CNS have several unique pharmacokinetic (PK) and pharmacodynamic (PD) properties, including active uptake mechanisms, low systemic exposure, long half-lives (t_{1/2}), accumulation, and gradual release from subcellular depots (Khorkova and Wahlestedt, 2017, Nat. Biotechnol. 35:249-63, of which entire content is incorporated herein by reference). CNS delivery of ASOs, such as Compound-A, is not gene therapy (i.e., therapies delivered or expressed using viral technology). Compound-A is a synthetically manufactured chemical product which the FDA categorizes as a small molecule. Compound-A is chemically synthesized and may not be regarded as a biologic.

### Example 18: Pharmacological Evaluation of the Therapeutic Agent in Non-Human Models

Initial target engagement, pharmacology, and efficacy studies with Compound-A were performed in mice, including both wild-type and a DS mouse model. The targeted non-productive splicing event in SCN1A is highly conserved across multiple species, including mice, non-human primates (NHPs), and humans. The target sequence for Compound-A is also identical across species.

Characterization of target engagement and pharmacology of Compound-A was done in wild-type mice. Neonate (postnatal day one) mice were administered a single dose of Compound-A by intracerebroventricular injection. On Day 5 of life the brains were isolated and were processed for RNA and protein. Treatment with Compound-A resulted in a dose-dependent reduction of non-productive Scn1a mRNA. Furthermore, the reduction of non-productive mRNA was associated with an increase of productive Scn1a mRNA and an increase in Na_{V}1.1 protein.

Compound-A pharmacology and efficacy were also investigated in transgenic mice with a heterozygous deletion of Scn1a. This model was created by introducing a targeted deletion in the first coding exon of the Scn1a gene; these mice exhibit many aspects of the DS phenotype including seizures and premature lethality and has been previously used to evaluate new AEDs for DS. Neonate (postnatal day two) and wild-type littermate controls were administered a single dose of either placebo (consisting of a phosphate-buffered solution) or Compound-A by intracerebroventricular injection. A single injection of Compound-A restored Na_{V}1.1 protein in DS mice to levels that are near those of the wild-type mice at both 7 and 14 weeks. Compound-A treated samples showed an increase in expression of the SCN1A gene, but not any of the other SCN family members. These results demonstrate that Compound-A is highly specific for SCN1A among the highly homologous family of sodium channel genes, indicating a low likelihood of off-target activities. In addition to an increase in Na_{V}1.1 protein, the administration of a single dose of Compound-A in DS mice resulted in a significant reduction in premature mortality. Treatment with Compound-A resulted in 97% survival of DS mice for the 90-day postnatal observation period compared with 23% survival of placebo-treated mice. The 90-day post-natal mouse is equivalent to a greater than 2-year-old human (Arzimanoglou et al., 2018, Pediatr Drugs. 20:249-64, incorporated herein by reference).

In sum, preclinical data obtained with Compound-A demonstrate proof-of-mechanism and clinical efficacy for Compound-A. Compound-A engages the target and elicits the predicted pharmacology in wild-type mice brain. Administration of a single dose of Compound-A in DS mice resulted in a significant reduction (p<0.0001) in premature mortality.

The pharmacology, distribution, and tolerability of Compound-A were also evaluated in cynomolgus monkeys. Pre-pubescent monkeys were administered a single dose of Compound-A or control solution by intrathecal (IT) injection at a dose range that coincides with the estimated therapeutic dose range and stays below the maximum tolerated dose based on tolerability in monkeys and published data for molecules of similar chemistry. The animals (n=3 for treatment groups and n=2 for control groups) were sacrificed at 2 days or 28 days after dosing. A two-fold increase in the Na_{V}1.1 protein was observed. The increase in Na_{V}1.1 was also correlated with the presence of Compound-A in brain tissue.

### Example 19: Pharmacological Evaluation of Therapeutic Agent in Human Patients

DS is a highly drug-resistant form of genetic epilepsy, associated with multiple severe comorbidities. The Worsening Stage (between 1 and 8 years of age) is a critical window for therapeutic intervention to reduce the occurrence and severity of comorbidities such as intellectual disability. It is therefore crucial to develop novel therapeutics for the treatment of DS that may be administered to the pediatric population.

The safety and PK endpoints are, for example, the safety profile and PK of single or multiple doses of Compound-A. Safety variables for analysis include, but are not limited to, the incidence, type, and severity of AEs, vital signs, ECG/Holter, laboratory and physical examination parameters. The PK parameters may be obtained by non-compartmental analysis from plasma concentrations of Compound-A that may include, but are not limited to: Maximum plasma concentration (Cmax), Time to maximum plasma concentration (Tmax), Area under the plasma concentration-time curve from time 0 to infinity and to the last measurable concentration (AUC_{0-∞}, AUC₀₋ₜ), and Fold change in Cmax and AUC compared with dose levels. Exposure of Compound-A in CSF may be assessed by measuring concentrations of Compound-A.

The efficacy endpoints are, for example, evidence of dose effect and comparison between baseline and the end of treatment in: (i) percentage change from baseline in convulsive seizure frequency (as measured by paper diary) calculated over 4-week time periods; (ii) proportion of patients with >50%, >75%, and 100% reduction (compared with baseline) in convulsive seizure frequency (as measured by paper diary) calculated over 4-week time periods; (iii) change from baseline in overall clinical status as measured by the following scales: Caregiver Global Impression of Change (CaGIC), Clinical Global Impression of Change (CGIC), and Change from baseline in the patient's quality of life as measured by the EuroQol-5D (Youth) (EQ-5D-Y) instrument.

Other efficacy endpoints include, but are not limited to, number and type of all convulsive and non-convulsive seizures, percentage change from baseline in total seizure frequency (as measured by paper diary) calculated over, e.g., 4-week time periods, proportion of patients with ≥50%, ≥75%, and 100% reduction (compared with baseline) in total seizure frequency (as measured by paper diary) calculated over, e.g., 4-week time periods, change in 10-20 electroencephalogram (EEG) parameters, total sleep time measured by actigraphy and as weekly sleep duration (hours) by sleep diary, convulsive seizure frequency as measured by, e.g., the Embrace2 wearable device, assessment of ambulation and gait as measured by the Gillette Functional Assessment Questionnaire (FAQ) 22-item skill set, and analysis of CSF, plasma, or serum samples for exploratory biomarkers (e.g., Na_{V}1.1, neurofilament light chain, etc.).

### Dose Levels

Single or multiple doses of Compound-A, e.g., 0.1, 0.5, 1, 2.5, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mg, is administered by intrathecal (IT) injection to patients with DS. The dose levels may be adjusted based on Safety Monitoring Committee (SMC) determination. Dose escalation between cohorts may be two-fold or lower increase for each dose level.

### Patient Population

Patients meeting the following criteria (Inclusion Criteria) may be eligible: (i) patient and/or authorized representative must be willing and able to give informed consent/assent and any authorizations required by local law for participation in the study; (ii) patient and their caregiver must be willing and able to comply with all protocol requirements; (iii) patient must be up to 18 years (inclusive) of age at Screening; (iv) patient must have DS as defined by: (a) onset prior to 12 months of age with recurrent focal motor or hemiconvulsive or generalized tonic-clonic seizures, which are often prolonged and triggered by hyperthermia, (b) no past history of causal magnetic resonance imaging (MRI) lesion (MRI not required to confirm absence of lesion), (c) no other known etiology, and (d) normal development at seizure onset; (v) patient must have a documented pathogenic, likely pathogenic variant, or variant of uncertain significance in the SCN1A gene associated with DS. Patients who have SCN1A testing results of negative (no variants of clinical significance identified) may not be enrolled; (vi) patient has had at least 2 prior treatments for epilepsy that either had lack of adequate seizure control (requiring an additional AED) or had to be discontinued due to an AE(s); (vii) patient must be experiencing 4 or more convulsive seizures (Hemiclonic, Focal with Motor Signs, Focal to Bilateral Tonic Clonic Convulsion, Generalized Tonic Clonic Convulsion, Tonic, Tonic/Atonic (Drop Attacks), and Clonic) during the initial Observation Period; (viii) patient must currently be taking at least one AED at a dose which has been stable for at least 4 weeks prior to Screening; (ix) all epilepsy medications or interventions for epilepsy (including ketogenic diet or vagal nerve stimulator) must have been stable (including product type, dose, and setting) for at least 4 weeks prior to Screening; (x) any marijuana- or cannabinoid-based product or medication is allowed but treatment must have been stable for at least 4 weeks prior to Screening, including supplier, ratio, and dose; and (xi) patient must meet age-appropriate institutional guidelines for IT drug administration procedures.

Patients meeting the following criteria (Exclusion Criteria) may not be eligible: (i) patient has one of the following mutations in the SCN1A gene: Thr226Met, Leu263Val, Val422Leu, Thr1174Ser, Trp1204Arg, Pro1345Ser, Gln1489Lys, Phe1499Leu, Arg1575Cys, Val1611Phe, Leu1624Pro, Arg1648Cys, Leu1649Gln, Leu1670Trp, Gly1674Arg, or Asp1866Tyr; (ii) patient has a known pathogenic mutation in another gene that causes epilepsy (the pathogenic mutation must be homozygous in cases of known recessive disease); (iii) patient is currently being treated with a sodium channel blocker as maintenance treatment including: phenytoin, carbamazepine, oxcarbazepine, lamotrigine, lacosamide, or rufinamide; (iv) patient has clinically significant unstable medical conditions other than epilepsy; (v) patient has had clinically relevant symptoms or a clinically significant illness in the 4 weeks prior to Screening or prior to dosing on Day 1, other than epilepsy; (vi) patient has a history of brain or spinal cord disease (other than epilepsy or DS) or a history of bacterial meningitis or brain malformation; (vii) patient has a spinal deformity or other condition that may alter the free flow of CSF or has an implanted CSF drainage shunt; (viii) patient has clinically significant (in the judgment of the Investigator) abnormal laboratory values at Screening or prior to dosing on Day 1; (ix) patient has AST or ALT >2.5-fold ULN, serum creatinine >ULN, or platelet count <the lower limit of normal at Screening and upon repeat testing; (x) patient has clinically relevant abnormalities in the 12-lead ECG measured at Screening or prior to dosing on Day 1; (xi) patient has a psychiatric or behavioral disorder which, in the opinion of the Investigator, may interfere with the patient's participation in the study; (xii) patient is currently taking, or in the past 4 weeks has taken, any anticoagulant (except aspirin); (xiii) female patients of childbearing potential and male patients whose partner is of childbearing potential, unless willing to ensure that they or their partner use effective contraception, for example, abstinence, oral contraception, double barrier, or intra-uterine device during the study and for 3 months thereafter; (xiv) patient who is pregnant, lactating, or planning pregnancy during the course of the study and for 3 months thereafter following the last dose of Compound-A; (xv) Patient who is currently enrolled in or has been part of a blinded clinical study involving an investigational product within 2 months prior to Screening; and (xvi) Patient has any other significant disease or disorder which, in the opinion of the Investigator, may either put the patient at risk because of participation in the study, may influence the results of the study, or may affect the patient's ability to participate in the study.

The mechanism of action of Compound-A may cause an increase in the levels of the gain-of-function mutated protein and treatment may lead to a worsening of symptoms. Patients carrying gain-of-function mutations in SCN1A may likely be excluded from the study because they do not meet the criteria for DS diagnosis.

### Administration of Therapeutic Agents

The drug product is a concentrate intended for dilution with artificial cerebral spinal fluid (aCSF) solution followed by intrathecal administration. The diluent, aCSF, for administration is supplied with the drug product. The drug product may be produced with sufficient fill volume to allow withdrawal of, e.g., 5.0 mL. The solution is a clear, colorless liquid that is essentially free of visible particles. Instructions regarding drug storage, preparation and dosing may be included.

Mode of Administration: diluted drug product may be administered as an IT slow bolus.

An exemplary drug product is provided in a kit comprised of 3 vials: 1 vial of drug product at an appropriate concentration; 1 vial of aCSF diluent; and 1 empty, sterile vial for mixing. It is suitably packaged in such a way as to protect the product from deterioration during transport and it is stored frozen at, e.g., −20° C. at the study site. Further information regarding storage and transport conditions may be provided. The study drug may be shipped on dry ice and stored on site at, e.g., −20° C.

In addition to the therapeutic agents as described herein, for example, Compound-A, patients may take at least one AED during the study, and any AEDs the patient takes may be maintained at a stable dose prior to Screening and after dosing. For some examples, patients may take at least one AED during the study, and any AEDs the patient takes may be maintained at a stable dose for at least, e.g., 4 weeks prior to Screening and for the first, e.g., 12 weeks after dosing, in addition to the therapeutic agents as described herein, for example, Compound-A. Exemplary medications or interventions for epilepsy include, but are not limited to, ketogenic diet, vagal nerve stimulator, cannabinoid or marijuana-derived products. Exemplary rescue medications allowed for home use include, but are not limited to, lorazepam, midazolam, and diazepam. Lorazepam may be used at 0.1-0.2 mg/kg as needed (PRN) intrabuccally, sublingually, or intravenously. Midazolam may be used at 0.1-0.2 mg/kg PRN intranasally, intrabuccally, or intravenously. Diazepam may be used at 0.2-0.5 mg/kg PRN rectally or intravenously.

The exemplary medications that may not be concomitantly used with the therapeutic agents as described herein, for example, Compound-A, include, but are not limited to, sodium channel blockers as maintenance treatment (e.g., phenytoin, carbamazepine, oxcarbazepine, lamotrigine, lacosamide, or rufinamide), anticoagulants (except aspirin), and any investigational product or device.

### Study Assessments

### Epilepsy Genetic Panel Testing:

Saliva samples are collected at Screening for determination of the patient's DS mutation and confirmation of eligibility. The samples are analyzed using a genetic epilepsy panel. The panel also analyzes genes associated with both syndromic and nonsyndromic causes of epilepsy (including SCN1A) and provides a comprehensive analysis for inherited epilepsy.

### Baseline Assessments:

Exemplary patient demographic and baseline characteristic data to be collected on all patients include, but are not limited to, age at Screening, sex, race/ethnicity, age of DS disease onset, and diagnostic and treatment history for DS.

### Example 20: Targeted Augmentation of Nuclear Gene Output (TANGO) of SCN1A Reduces Seizures and Rescues Parvalbumin Positive Interneuron Firing Frequency in a Mouse Model of Dravet Syndrome

Dravet syndrome (DS) is a severe and progressive developmental and epileptic encephalopathy characterized by high seizure frequency and severity, intellectual disability, and a high risk of sudden unexpected death in epilepsy (SUDEP). Approximately 85% of DS cases are characterized by spontaneous, heterozygous loss of function mutations in the SCN1A gene which encodes the voltage-gated sodium channel a subunit Na_{V}1.1. A novel therapeutic approach was developed to treat DS using antisense oligomers (ASOs) to increase the endogenous expression of Scn1a mRNA and Na_{V}1.1 protein. This approach was tested using the F2:129S-Scn1a^{+/-}×C57BL/6J DS mouse model that has been shown previously to recapitulate many patient phenotypes.

### Methods

Targeted Augmentation of Nuclear Gene Output (TANGO), which modulates naturally occurring, non-productive splicing events to increase target protein expression, was used to screen and identify an ASO (Compound-A) that can increase productive Scn1a mRNA and Na_{V}1.1 protein in vivo. 20 μg TANGO ASO or PBS control was administered to DS mice and wildtype (WT) littermates by single intracerebroventricular (ICV) injection at postnatal day (P)2. Mice were monitored by electroencephalography (EEG) pre (P13-19) and post (P20-40) weaning. DS mice were also crossed with WT mice hemizygous for a parvalbumin (PV)-tdTomato fluorescent reporter to produce DS mice expressing the tdTomato specifically in PV expressing interneurons. These mice received 20 μg ASO or PBS by ICV administration at P2 and then electrophysiological recordings were taken from tdTomato expressing cells in the somatosensory cortex between P17 and 23.

### Results

No seizures were detected in any of the mice prior to day 16. Between P16-19, ASO-injected DS mice continued seizure free (0/5 animals) whereas 50% (4/8 animals) of PBS-injected control DS mice had seizures. When assessed after weaning (P20-40), 2/11 P2 ASO injected DS mice developed seizures and one animal died while 9/11 P2 PBS-injected control DS mice developed seizures and seven animals died. The firing frequency of PV interneurons in DS mice assessed between P17 to P23 was significantly impaired. DS PV interneurons fired at a significantly (P<0.05) lower frequency than WT PV interneurons over a range of current injection steps. After ASO treatment at P2, the firing frequency of PV interneurons in DS mice was increased and was no longer significantly different from WT levels.

### CONCLUSIONS

These results provide evidence that increased Scn1a and Na_{V}1.1 expression achieved using a TANGO ASO can greatly decrease seizures and death rates in a mouse model of SCN1A-linked DS. Further, the current data support the hypothesis that the improvement in DS phenotype is in part due to restoration of excitability of PV expressing interneurons.

### Example 21: Targeted Augmentation of Nuclear Gene Output (TANGO) of SCN1A Reduces Seizures and Rescues Parvalbumin Positive Interneuron Firing Frequency in a Mouse Model of Dravet Syndrome

Dravet syndrome (DS) is a severe developmental and epileptic encephalopathy. DS is caused primarily by physiologically loss-of-function SCN1A mutations resulting in hypofunction of inhibitory interneurons. Patients suffer refractory seizures, cognitive and motor impairments, and have a substantial risk for SUDEP. There is a demand for therapeutic strategies that directly address genetic cause of disease.

Compound-A reduced seizure frequency and extended survival in DS mice with no significant deleterious effects observed in WT mice. Treatment with Compound-A rescues neuronal excitability of parvalbumin-positive inhibitory interneurons in DS mice, which supports the hypothesis that restoration of excitability to inhibitory interneurons is a viable approach toward rescuing DS mice from seizures and death.

Potential future evaluations: collect electrophysiology recordings of voltage-gated sodium channel activity; explore effects on other inhibitory interneuron populations (SST, VIP, etc.); examine impact on network excitability (synaptic inhibition, etc.).

### Example 22: Safety and Pharmacokinetics of Antisense Oligonucleotide Compound-A in Children and Adolescents with Dravet Syndrome: Single Ascending Dose Design for the Open-Label Phase 1/2a MONARCH and ADMIRAL Clinical Studies

Condition or disease: Dravet Syndrome.
Intervention/treatment: Drug: Compound-A
Phase: Phase 1, Phase 2
Ages Eligible for Study: 2 Years to 18 Years (Child, Adult);
Sexes Eligible for Study: All;
Accepts Healthy Volunteers: No

### Rationale

Dravet syndrome (DS) is a severe and progressive genetic epilepsy characterized by frequent, prolonged, and refractory seizures, beginning within the first year of life. It is characterized by high seizure frequency and severity, intellectual disability, and a high risk of sudden unexpected death in epilepsy. Available therapies do not adequately control seizures in 90% of DS patients, and they do not address other aspects of the disease, including intellectual disability, developmental delays, motor and speech impairment, behavioral problems, sleep abnormalities, and an increased risk of sudden unexpected death in epilepsy. Complications of the disease often contribute to a poor quality of life for patients and their caregivers. In approximately 85% of cases, DS is characterized by spontaneous, heterozygous loss of function mutations in the SCN1A gene, which encodes the voltage-gated sodium channel type 1α subunit (Na_{V}1.1). Upregulating Na_{V}1.1 may restore functioning neurons and prevent seizures and reduce non-seizure related comorbidities in DS.

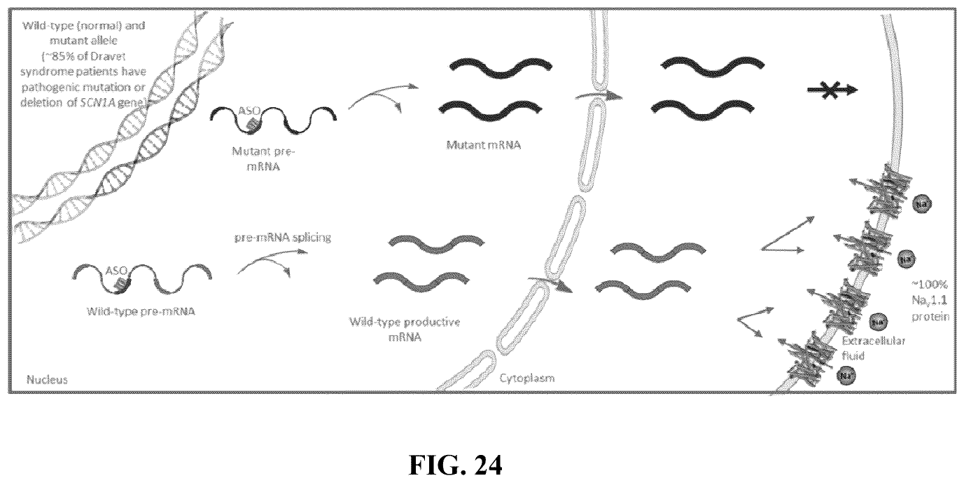

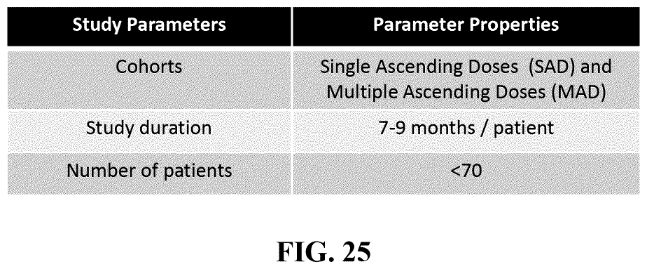

Compound-A is an investigational new medicine for the treatment of Dravet syndrome. Compound-A is an antisense oligonucleotide treatment using a unique platform, Targeted Augmentation of Nuclear Gene Output (TANGO) (FIG. 24) that exploits naturally occurring nonproductive splicing events to increase Na_{V}1.1 protein expression. Compound-A is an antisense oligonucleotide (ASO) that is intended to increase the level of productive SCN1A messenger RNA (mRNA) and consequently increase the expression of the sodium channel Na_{V}1.1 protein. This RNA-based approach is not gene therapy, but rather RNA modulation, as it does not manipulate nor insert genetic deoxyribonucleic acid (DNA). Compound-A is designed to upregulate Na_{V}1.1 protein expression from the nonmutant (wild-type) copy of the SCN1A gene to restore physiological Na_{V}1.1 levels. Na_{V}1.1 levels are reduced in people with Dravet syndrome. Stoke has generated preclinical data demonstrating proof-of-mechanism for Compound-A, which is also used interchangeably with “ASO-1.”

In DS, patients have one functional gene copy (orange) and one mutated copy (red), resulting in half as much protein as needed to maintain health. These genes are transcribed into pre-messenger RNA (pre-mRNA); most pre-mRNA is productive, becoming a template for protein production, but some is non-productive pre-mRNA. Synthesized ASOs (green) bind to specific stretches of pre-mRNA, reducing the synthesis of non-productive mRNA and increasing the synthesis of productive mRNA. The increased levels of productive mRNA from the functional gene copy increase protein production, thereby restoring the target protein to near normal levels.

Current treatments focus on seizure control. Compound-A may be the first precision medicine approach for DS. Compound-A has the potential to be the first disease-modifying therapy to address the genetic cause of DS by restoring physiological Na_{V}1.1 levels and reducing both occurrence of seizures and significant non-seizure comorbidities. This clinical study aims to primarily assess the safety, tolerability and pharmacokinetics of intrathecally administered Compound-A. Secondary objectives aim to evaluate the effect of Compound-A on convulsive seizure frequency, overall clinical status and quality of life in Dravet syndrome patients. The dose implications of this study may better inform future clinical trials on the appropriate and effective dosing for efficacy measures.

### Study Design:

Open-label, Single and Multiple Ascending Doses (SAD and MAD) of Compound-A in 2-18 y.
Study duration: 7-9 months/patient; Number of patients <80;
Sites: US; UK, Scotland, Ireland among others;
Cohorts: This phase 1/2a open-label single ascending dose study includes patients aged 2-18 years with disease onset prior to 12 months of age with recurrent seizures (focal motor, hemiconvulsive or generalized tonic-clonic) and genetically confirmed SCN1A variant.
Inclusion Criteria: Must have DS with onset prior to 12 months of age with recurrent focal motor or hemiconvulsive or generalized tonic-clonic seizures, which are often prolonged and triggered by hyperthermia; No history of causal MRI lesion; No other known etiology; Normal development at seizure onset; Documented pathogenic, likely pathogenic variant, or variant of uncertain significance in the SCN1A gene associated with DS; Had at least 2 treatments for epilepsy that either had lack of adequate seizure control (requiring an additional AED) or had to be discontinued due to an AE(s); Currently be taking at least one AED at a dose which has been stable for at least 4 weeks prior to Screening; All epilepsy medications or interventions for epilepsy (including ketogenic diet or vagal nerve stimulator) must have been stable (including product type, dose, and setting) for at least 4 weeks prior to Screening.
Exclusion Criteria: Known pathogenic mutation in another gene that causes epilepsy; Currently being treated with an antiepileptic drug acting primarily as a sodium channel blocker including: phenytoin, carbamazepine, oxcarbazepine, lamotrigine, lacosamide, or rufinamide; Clinically significant unstable medical conditions other than epilepsy; Has had clinically relevant symptoms or a clinically significant illness in the 4 weeks prior to Screening or prior to dosing on Day 1, other than epilepsy; Any other significant disease or disorder which, in the opinion of the Investigator, may either put the patient at risk because of participation in the study, may influence the results of the study, or may affect the patient's ability to participate in the study.
Dosing: Dose escalation based on safety and tolerability assessed by Safety Committee (with external reviewers), Dosing begins in 13 to 18 y cohorts, with internal safety team approving dosing in 2 to 12 y.

### A. MONARCH and ADMIRAL Studies: Single Ascending Dose Arm

Enrollment of patients in two age groups. A Sentinel group of patients aged 13 to 18 years of age, inclusive, and an expanded group of patients 2 to 12 years of age were to receive single doses. There was an option to dose up to 6 additional patients at each dose level and an option to expand the maximum tolerated dose level with 5 additional patients. In Single Ascending Doses arm (SAD), ASO-1 drug product is an antisense oligonucleotide administered as an intrathecal injection. Four dose levels were evaluated (10 mg, 20 mg, 30 mg, 45 mg and 70 mg). In the MONARCH study, SAD regimes at 10, 20, 30 mg and 45 mg dosages were administered to an expanded group of patients 2 to 12 years of age and 13-18 years of age. In the ADMIRAL study, SAD regimes at 30, 45, and 70 mg were administered to an expanded group of patients 2 to 12 years of age and 13-18 years of age. Dose escalation was based on safety and tolerability assessment by the Safety Monitoring Committee (including external reviewers). Dosing began in each cohort in 13- to 18-year-olds and an internal safety team approved dosing in younger patients (2-12 years).

### B. MONARCH Study: Multiple Ascending Doses Arm

Enrollment of patients in two age groups. A Sentinel group of 2 patients aged 13 to 18 years of age, inclusive, and an expanded group of 2 patients 2 to 12 years of age to receive multiple doses. There will be an option to dose up to 6 additional patients at each dose level and an option to expand the maximum tolerated dose level with 10 additional patients. In the Multiple Ascending Doses (MAD) arm, ASO-1 drug product is an antisense oligonucleotide administered as an intrathecal injection. Three dose levels will be evaluated (20 mg, 30 mg and 45 mg). In the MONARCH study, MAD regimes at 20, 30 mg and 45 mg dosages were administered to an expanded group of patients 2 to 12 years of age and 13-18 years of age.

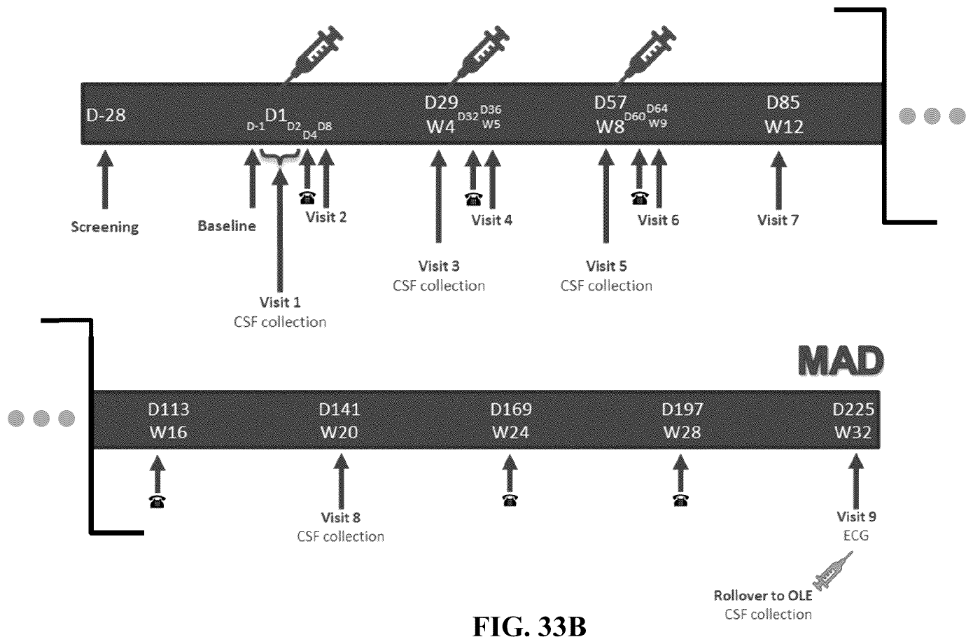

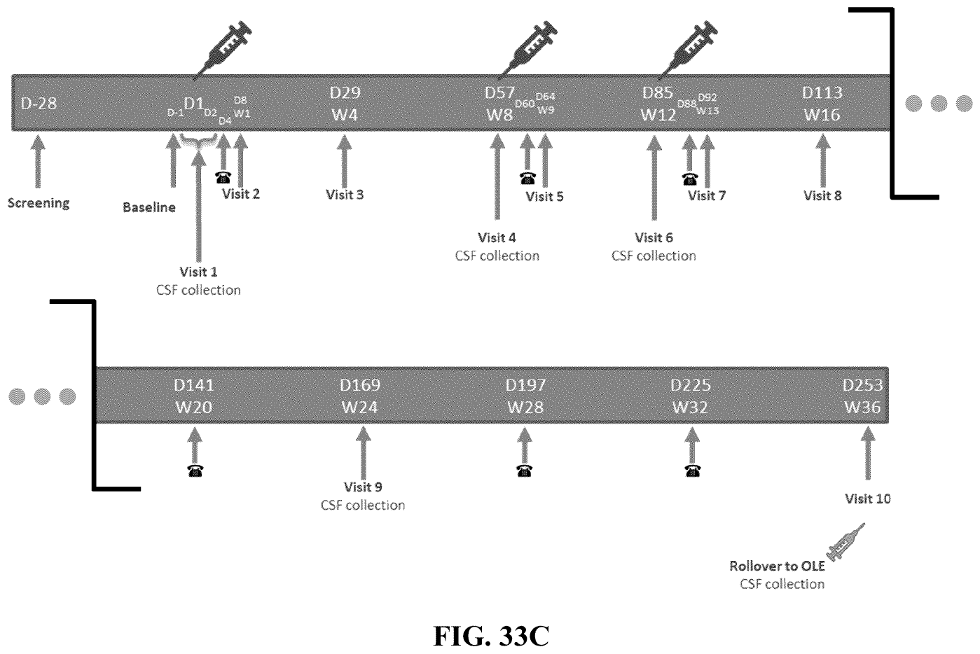

FIGS. 33A-C shows the study design for the Phase1/2a clinical trials for ASO-1. FIG. 33A shows the study design and dosing schedule using the Single Ascending Dose (SAD) regime (MONARCH) before patients can become eligible to enroll in the LONGWING OLE. FIG. 33B shows the study design and dosing schedule using the Multiple Ascending Dose regime (MONARCH) before patients can become eligible to enroll in the LONGWING OLE. FIG. 33C shows the study design and dosing schedule using the Multiple Ascending Dose regime (ADMIRAL) before patients can become eligible to enroll in the LONGWING OLE. Study protocol for ADMIRAL was amended to allow investigators to decide whether to administer two or three doses of ASO-1 (70 mg) in the ADMIRAL study before patients would be eligible to enroll in the LONGWING OLE.

FIGS. 33A-C shows the workflow of the SAD and MAD study assessments in the MONARCH and ADMIRAL studies. Basically, for each patient in the study, there was a baseline period for monitoring seizure from Day −28 and Day −1 (in reference to 1^{st} dose of study drug), and the patient was monitored continuously (adverse effect monitoring, physical examination, clinical laboratories, and plasma PK) for 225 days post administration in the SAD and MAD arms of the MONARCH study and 252 days in the SAD arm of the ADMIRAL study. Study drug, Compound-A was administered on Day 1 in SAD arm of the MONARCH study. Compound A was administered on Day 1, Day 29, and Day 57 in MAD arm of the MONARCH study. Compound A was administered on Day 1, Day 57, and Day 85 in SAD arm of the ADMIRAL study. Baseline seizure diary was collected in the periods of Day −28 to Day −1, Day 1 to Day 28, Day 29 to Day 56, Day 57-Day 84, Day 85-Day 112, Day 113 to Day 140, Day 141 to Day 168, Day 169 to Day 196, Day 197 to Day 224 in the MONARCH Study. Baseline seizure diary was collected in the periods of Day −28 to Day −1, Day 1 to Day 28, Day 29 to Day 56, Day 57 to Day 84, Day 85 to Day 112, Day 113 to Day 140, Day 141 to Day 168, Day 169 to Day 196, Day 197 to Day 224, and Day 225 to Day 252 in the ADMIRAL Study.

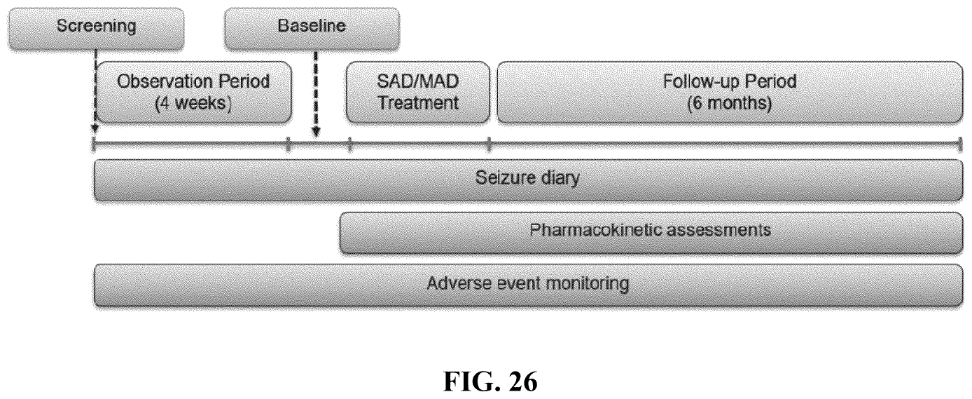

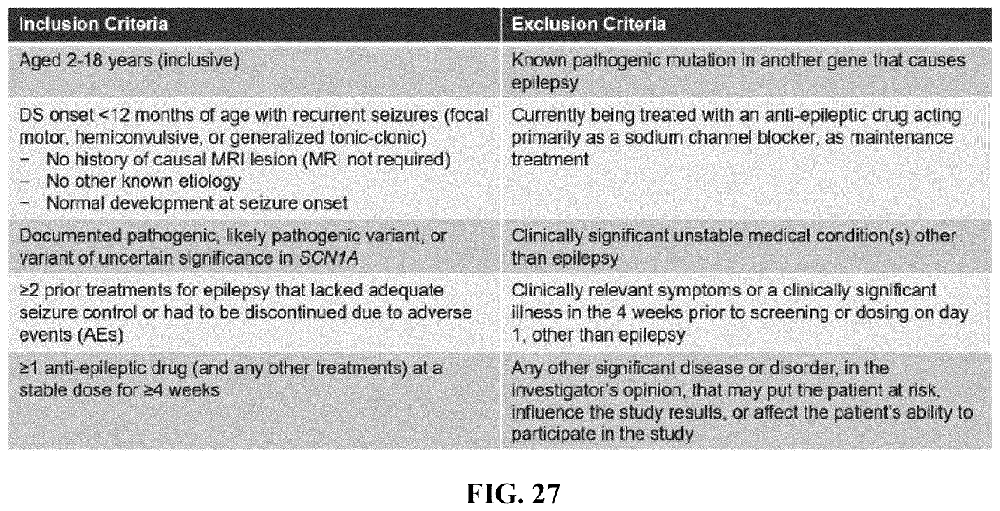

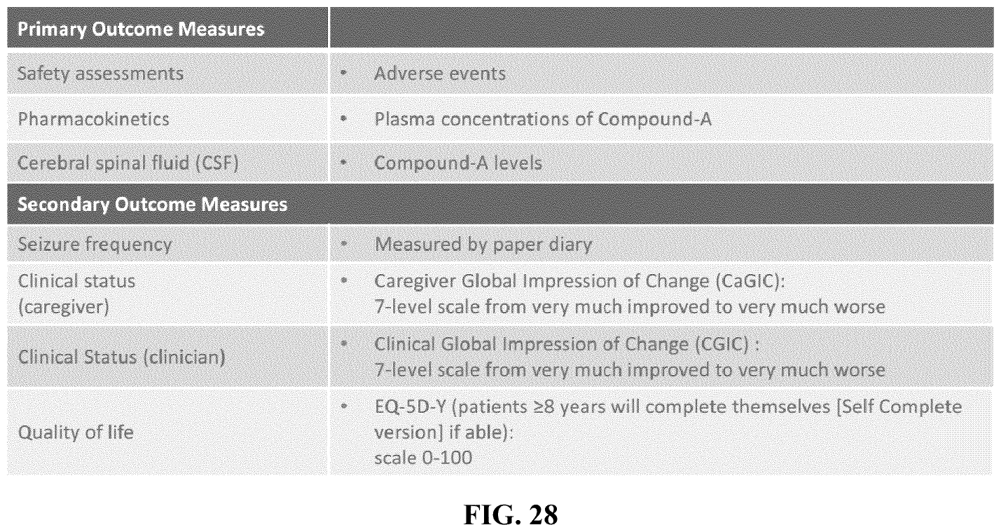

Study visits include the following (FIG. 26): screening visit; a 4-week observation period: no change to current anti-epileptic therapy, ketogenic diet, or vagal nerve stimulator settings, caregivers track child's seizure frequency during this period; Baseline visit: blood and urine analyses, Quality of Life, neurological, and general pediatric assessments; Inpatient treatment period: patients are admitted on the day of dosing and discharged after completing post-dose assessments: all patients will receive intrathecal administration of Compound-A; 3 month and 6-month follow-up period. Patients who complete the study will have the option to receive Compound-A in an open-label extension study if they meet enrollment criteria.

Two cohorts based on age (2-12 and 13-18 years) will be administered a single dose or multiple doses of Compound-A intrathecally (IT). Each cohort will enroll up to 4 patients with an option to dose up to 6 additional patients per cohort at the same level based on clinical assessment for safety evaluation. All patients will have a 28-day observation period evaluating seizure frequency. On Day 1, patients undergo cerebral spinal fluid (CSF) collection followed by a single IT administration of Compound-A and 24-hour post-dose assessment. There will be a 1-year follow-up period after dosing. Adverse events are monitored throughout the study. Plasma and CSF will be collected at multiple timepoints. Patients will also keep a seizure/sleep diary the full duration of the study. The demographics for the 74 patients treated with >=1 dose of ASO-1 in the MONARCH and ADMIRAL studies are shown in Table 11. Enrolled patients have severe disease and are refractory to Standard treatment.

### C. Outcome Measures:

### Primary Outcome Measures:

1. Incidence proportion of adverse events [Time Frame: Screening until 6 months after single drug dosing Safety of Compound-A will be evaluated by the proportion of subjects experiencing Adverse Events, Serious Adverse Events, and Adverse Events leading to drug discontinuation.
2. Pharmacokinetic (PK) Parameters [Time Frame: Screening until 6 months after single drug dosing] Analysis of plasma concentrations of Compound-A
3. Exposure of Compound-A in Cerebrospinal Fluid (CSF) [Time Frame: Screening until 6 months after single drug dosing] Measurement of Compound-A concentrations

### Secondary Outcome Measures:

1. Measurement of seizure frequency [Time Frame: Screening until 6 months after single drug dosing] Measured by paper diary
2. Change in clinical status [Time Frame: Screening until 6 months after single drug dosing] Change from baseline in overall clinical status as measured by the Caregiver Global Impression of Change (CaGIC).
Values of scales: a. Very much improved; b. Much improved; c. Minimally improved; d. No change; e. Minimally worse; f. Much worse; g. Very much worse
3. Change in clinical status [Time Frame: Screening until 7 months after single drug dosing] Change from baseline in overall clinical status as measured by the Clinical Global Impression of Change (CGIC)
Values of scales: a. Very much improved; b. Much improved; c. Minimally improved; d. No change; e. Minimally worse; f. Much worse; g. Very much worse
4. Measurement of Quality of Life [Time Frame: Screening until 6 months after single drug dosing] Change from baseline in the patient health is measured by the EuroQOL quality of life questionnaire, youth version (EQ-5D-Y) instrument. The scale is scored from 0-100. The reference to a high score indicates a better outcome of quality of life.

### Results

This study will provide insight into the safety, tolerability, and pharmacokinetic profile of ascending doses of Compound-A in DS patients. In addition, the impact of Compound-A on frequency of convulsive seizures and quality of life may indicate the initial clinical effect of the individual doses.

### Example 23: Interim Safety, PK, CSF Exposure, and Seizure Frequency Data from a Phase 1/2a Study of Compound-A in Children and Adolescents with Dravet Syndrome (DS)

This example describes some interim data obtained from a Phase 1/2a study of Compound-A in children and adolescents with DS. The data presented here illustrate the safety, pharmacokinetic, and cerebrospinal fluid (CSF) exposure features of Compound-A in the tested subjects and their seizure frequency before and after administration of Compound-A. Patients in this study have severe disease. The interim data presented here show that single doses of Compound-A up to 30 mg and multiple doses of 20 mg were well-tolerated, and there were no safety concerns related to study drug. Moreover, dose-dependent increases in plasma exposure and CSF concentration were observed, and it was found that seizures were reduced in 12 of 17 (70.6%), including all 2- to 12-year-old patients, at days 29-84 as compared to baseline period.

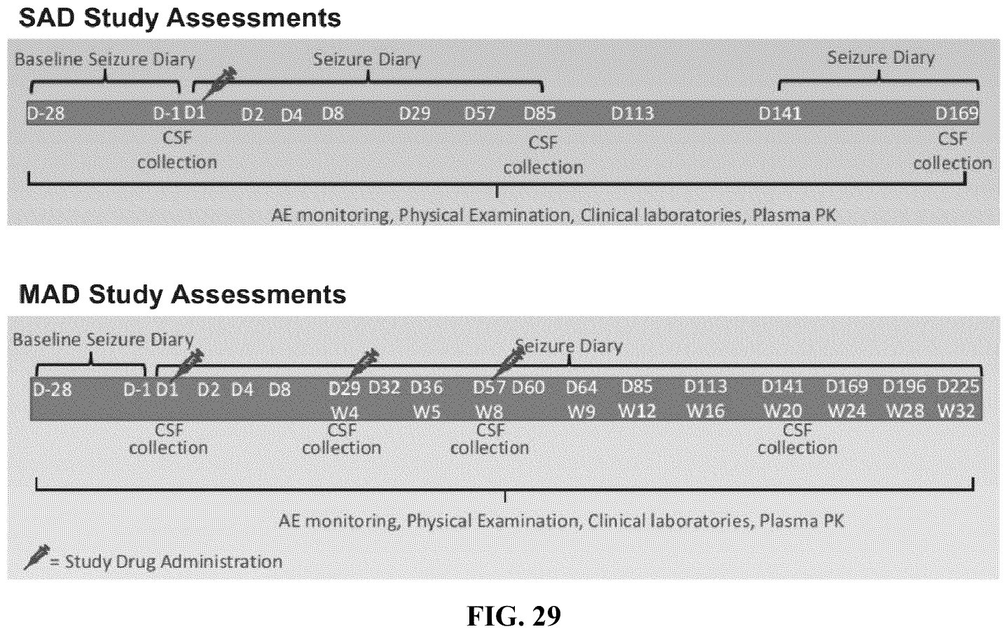

FIG. 29 shows the workflow of the SAD and MAD study assessments. Basically, for each patient in the study, there was a baseline period for monitoring seizure from Day −28 and Day −1 (in reference to 1^{st} dose of study drug), and the patient was monitored continuously (adverse effect monitoring, physical examination, clinical laboratories, and plasma PK) for 169 days post administration in SAD study, and 225 days in MAD study. Study drug, Compound-A was administered on Day 1 in SAD study, was administered on Day 1, Day 29, and Day 57 in MAD study. Baseline seizure diary was collected in the periods of Day −28 to Day −1, Day 1 to Day 85, and Day 141 to Day 169 in SAD study, and in the periods of Day −28 to Day −1, and Day 1 to Day 225 in MAD study. CSF collection was performed on Day 1, Day 85, and Day 169 in SAD study, and on Day 1, Day 29/Week 4, Day 57/Week 8, and Day 141/Week 20 in MAD study.

Cutoff date for data presented in this example was Oct. 19, 2021, after all patients in Cohort A3 (30 mg SAD) completed visit 5 (day 85) and Cohort B1 (20 mg MAD) completed visit 7 (week 12). All received ≥1 dose of Compound-A.

Study Object. Primary Assessments include Safety and Tolerability (Adverse events (AEs), vital signs, physical examination, electrocardiogram, laboratories), Pharmacokinetics (PK) (Compound-A plasma concentrations), and Cerebrospinal Fluid (CSF) Exposure (Compound-A CSF concentrations). Secondary Assessments include Convulsive seizure frequency (Daily paper seizure diary), Overall Clinical Status and Quality of Life (Caregiver and Clinical Global Impression of Change; EQ-5D-Y).

Table 9 below summarizes the demographics of the tested subjects and the safety evaluation after they received administration of Compound-A. The most common treatment-emergent adverse effects (TEAEs) include headache (reported in 7 patients), vomiting (6 patients), seizure (5 patients), irritability (4 patients), back pain (3 patients), fall (3 patients), and pyrexia (3 patients). In addition, overall, there was no new clinically significant weakness reported on physical exam, there was no increase in seizures identified in 1 hour EEG recorded ˜24 hours post-dose, and there were no clinically significant changes in laboratories assessed as related to study drug.

TABLE 9
Summary of Demographics and Safety

| SAD | | | MAD | |
|---|---|---|---|---|
| A1 (10 | A2 (20 | A3 (30 | B1 (20 | |
| mg) | mg) | mg) | mg) | Total |

Age at Screening, y

| | | | | | |
|---|---|---|---|---|---|
| n | 5 | 4 | 7 | 6 | 22 |
| Mean (SD) | 11.2 | 10.8 | 9.1 | 13.7 | 11.1 |
| | (5.89) | (5.19) | (5.90) | (5.13) | (5.46) |
| Median (min, max) | 13.0 | 10.0 | 10.0 | 16.0 | 13.0 |
| | (2, 18) | (6, 17) | (2, 16) | (4, 17) | (2, 18) |

Age Group, n (%)

| | | | | | |
|---|---|---|---|---|---|
| 2-12 y | 2 (40) | 2 (50) | 4 (57.1) | 2 (33.3) | 10 (45.5) |
| 13-18 y | 3 (60) | 2 (50) | 3 (42.9) | 4 (66.7) | 12 (54.5) |

# patients (all ages) with TEAEs (treatment-emergent adverse effects)

| | | | | | |
|---|---|---|---|---|---|
| TEAEs, n(%) | 4 (80.0) | 4 (100.0) | 7 (100.0) | 6 (100.0) | 21 (95.5) |
| TEAEs, related to study drug, n (%) | 2 (40.0) | 1 (25.0) | 0 | 1 (16.7) | 4 (18.2) |
| >=Grade 3 TEAEs, n (%) | 0 | 1 (25.0) | 1 (14.3) | 1 (16.7) | 3 (13.6) |
| >=Grade 3 TEAEs, related to study | 0 | 0 | 0 | 0 | 0 |
| drug, n (%) | | | | | |
| Serious TEAEs, n (%) | 1 (20.0) | 1 (25.0) | 1 (14.3) | 2 (33.3) | 5 (22.7) |
| Serious TEAEs, related to study | 0 | 0 | 0 | 0 | 0 |
| drug, n (%) | | | | | |
| TEAEs leading to study withdrawal | 0 | 0 | 0 | 0 | 0 |
| or death, n (%) | | | | | |

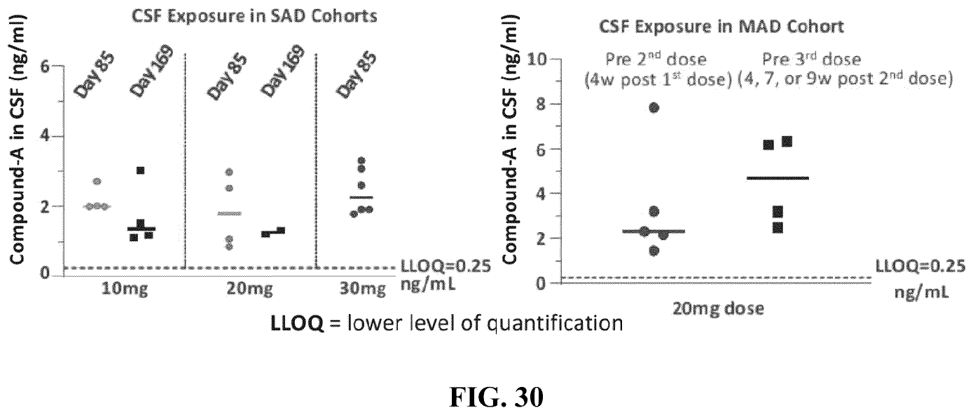

Table 10 below summarizes the pharmacokinetic results in each cohort, and FIG. 30 shows two plots summarizing CSF exposure of Compound-A in SAD cohorts and MAD cohorts, respectively. Dose-dependent increases in plasma exposure and CSF concentration were observed. Plasma AUCₗₐₛₜ was similar for the 20 mg SAD cohort and 1^{st} dose in the 20 mg MAD cohort. Last collection until which Compound-A CSF levels were detected was on day 169 for 10 and 20 mg, and day 85 for 30 mg. Overall, mean CSF concentration at day 85 increased from 10 to 30 mg. Mean CSF levels post 2^{nd} dose were higher compared to levels post 1^{st} dose indicating accumulation of Compound-A in CNS tissues with repeated monthly dosing.

TABLE 10
Plasma PK parameters of Compound A

| | Plasma PK parameters | |
|---|---|---|
| | | AUCₗₐₛₜ (h*ng/ml) |
| Dose (mg) | N | (mean ± SD) |
| SAD 10 | 4 | 2450 ± 1690 |
| SAD 20 | 4 | 6460 ± 2820 |
| SAD 30 | 7 | 15300 ± 12100 |
| MAD 20 (dose 1) | 6 | 4450 ± 3110 |

Observed baseline convulsive seizure frequency over a period of 28 days in the tested subjects was calculated, with a Mean (SD) of 51.41 (131.4), and Median (min, max) of 16 (1.0, 630.0). 90.9% patients used at least three concomitant anti-seizure medications as maintenance therapy, and 72.7% used at least four concomitant anti-seizure medications as maintenance therapy. Most common medications were clobazam (68.2%) and fenfluramine (54.5%).

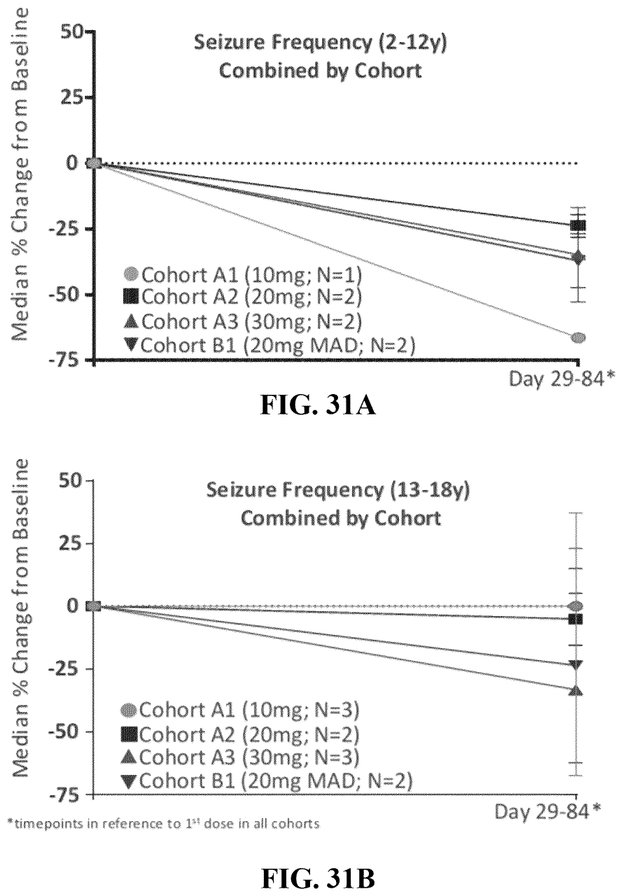

FIGS. 31A-31B show two plots summarizing mean percent change in seizure frequency from baseline level to the period between Day 29 and 84 post administration of Compound-A (in reference to 1^{st} dose, Day 29-84) in 2- to 12-year-old subjects (FIG. 31A) and 13- to 18-year-old (FIG. 31B), all combined by cohort. In 2- to 12-year-old subjects in all cohorts, there was a reduction in seizure frequency as measured: about 20% in Cohort A2 (20 mg, N=2), about 35% in both Cohort A3 (30 mg; N=2) and Cohort B1 (20 mg MAD, N=2), and about 70% in Cohort A1 (10 mg; N=1). In 13-18 y subjects, there was about 25% reduction in seizure frequency in Cohort B1 (20 mg MAD, N=2), and about 35% in Cohort A3 (30 mg; N=3). Note that when analyzing the seizure frequency data, Cohort A1 excludes patient with incorrect dosing, Cohort A3 excludes patients meeting minimum seizure count during observation but not during defined baseline; and Cohort B1 excludes patients who received only 2 doses prior to D84. Overall, seizures were reduced in 12 of 17 (70.6%), including all 2- to 12-year-old patients, at days 29-84 vs baseline period.

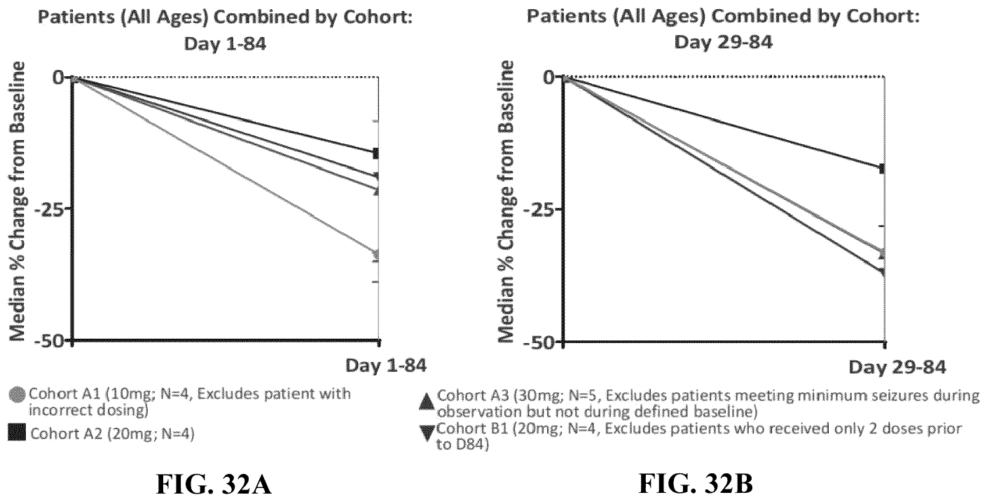

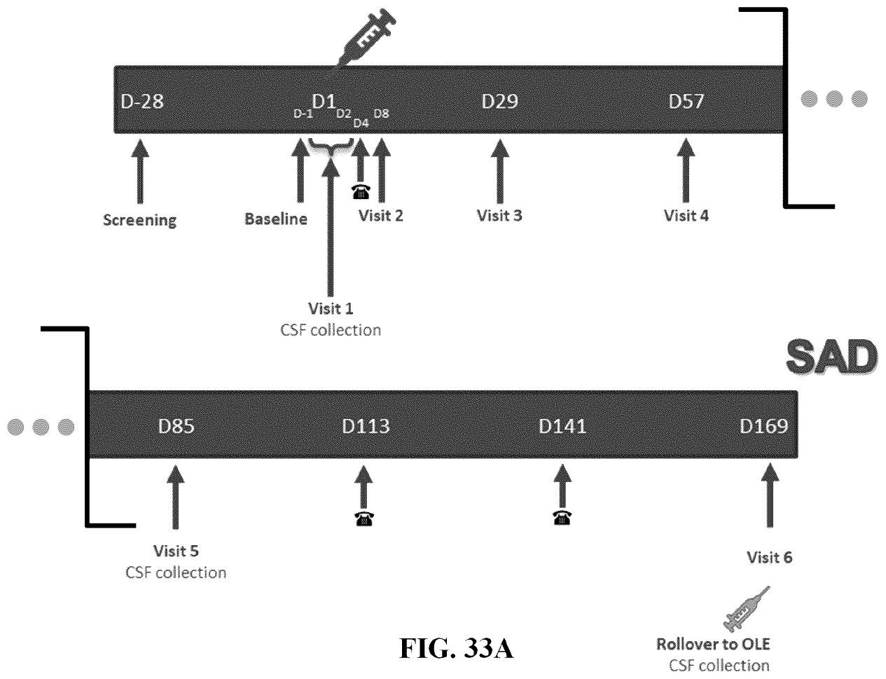

FIGS. 32A-32B show two plots summarizing mean percent change in seizure frequency from baseline in patients of all ages as combined by cohort in the period between Day 1 and Day 84 (Day 1-84) post administration (FIG. 32A), and in the period between Day 29-84 (Day 29-84) post administration (FIG. 32B).

### Example 24: Interim Safety, PK, CSF Exposure, and Seizure Frequency Data from a Phase 1/2a Study of Compound-A in Children and Adolescents with Dravet Syndrome (DS)

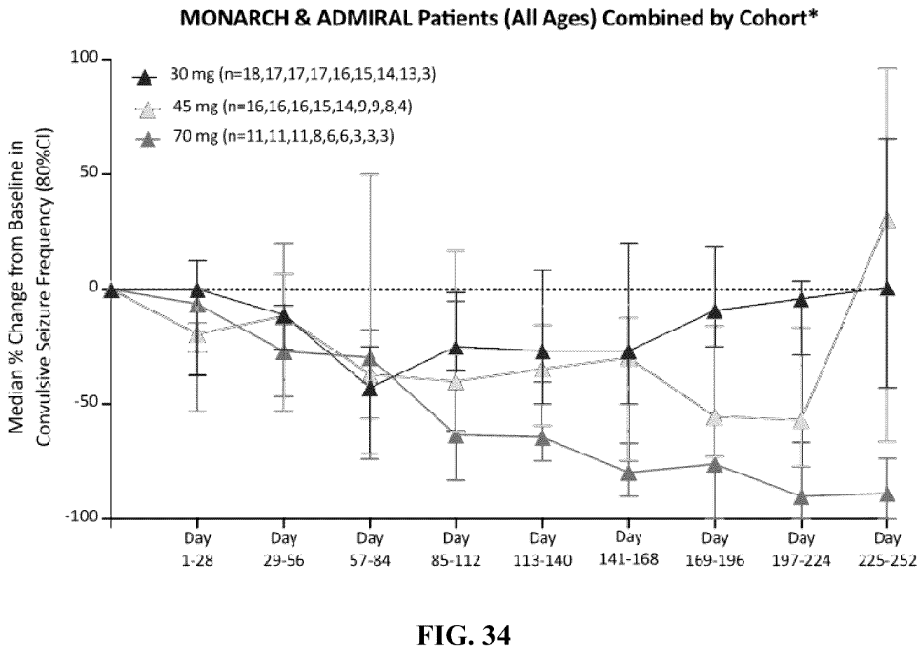

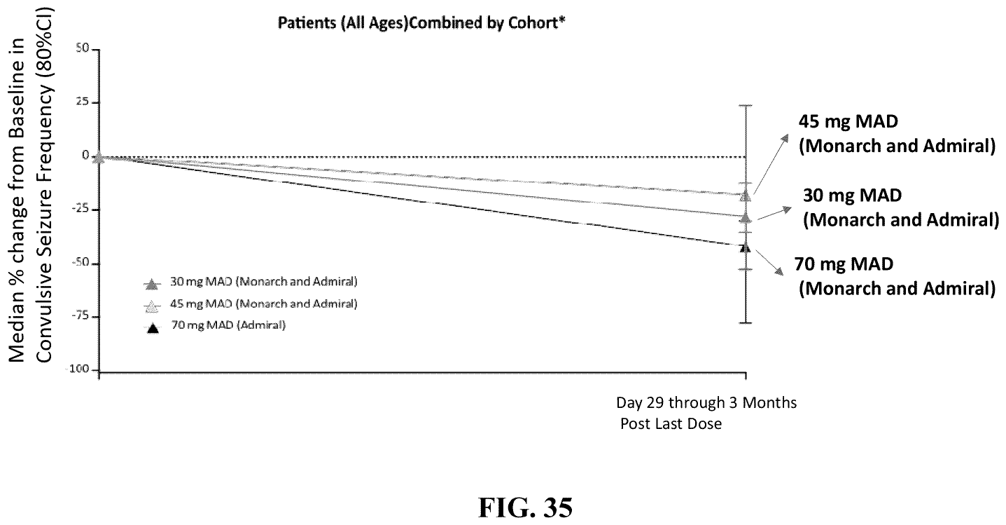

FIGS. 33A-C provide the study design for the Phase1/2a clinical trials for ASO-1. FIG. 34 shows a plot summarizing mean percent change in seizure frequency from baseline in patients of all ages receiving 30 mg, 45 mg of ASO-1, and combined by cohort in the period between Day 1 and Day 225 post administration in the MONARCH study (and in the period between Day 1 and Day 252 post administration in the ADMIRAL study). Overall, there was an observable beneficial effect of ASO-1 on convulsive Seizure Frequency in the 30 mg, 45 mg, and 70 mg Multiple Dose Cohorts (FIG. 34), with an average reduction of about 50%-60% (at the Day 225 period) in patients receiving the 45 mg dose, an average reduction of about 30% in patients receiving the 30 mg dose (at the Day 169 mark), and an average reduction of about 90% in patients receiving the 70 mg dose (at the end of the Day 252 mark). When measured through the period of Day 29 to 3-months post last dose administration, there was an average reduction of about 42% in patients receiving the 70 mg dose, an average reduction of about 30% in patients receiving the 45 mg dose, and an average reduction of about 18% in patients receiving the 30 mg dose (FIG. 35).

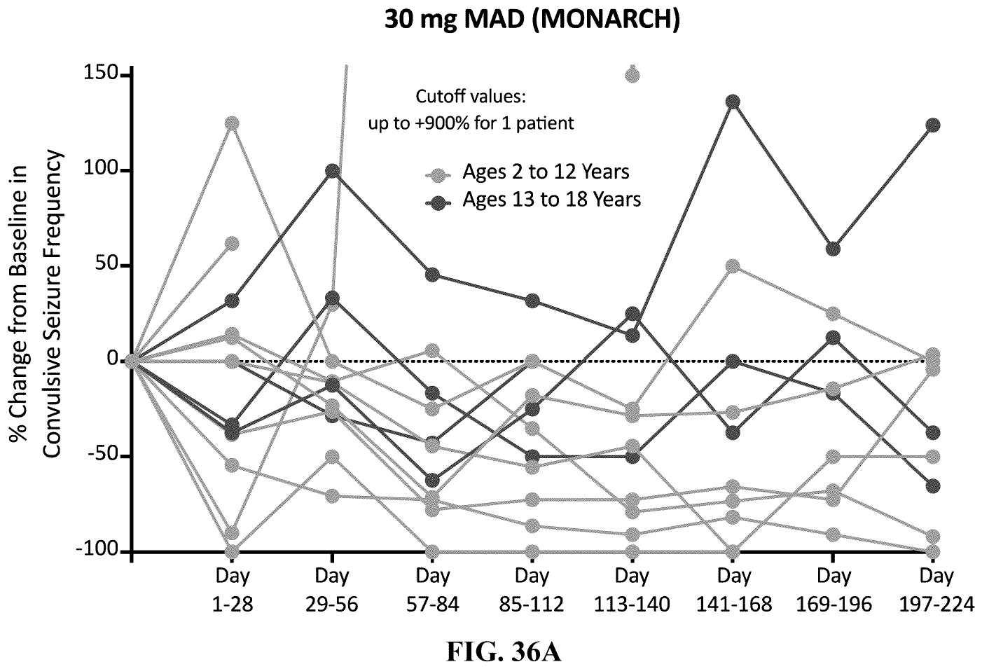

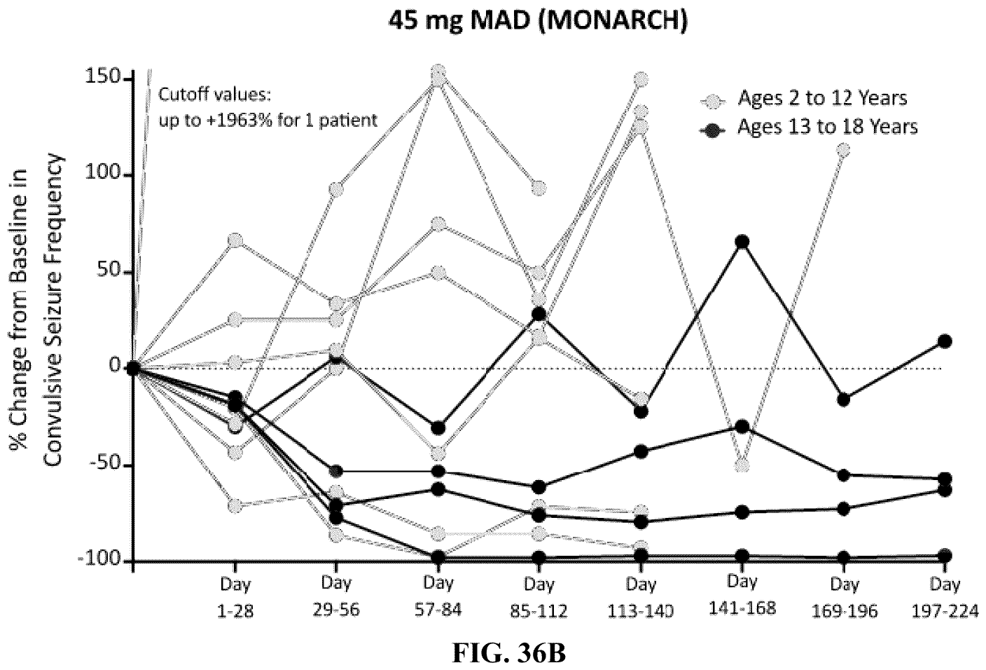

FIGS. 36A-36B show plots summarizing the mean percent change in seizure frequency from baseline in patients between ages 2-12 and between ages 13-18 years receiving 30 mg or 45 mg of ASO-1 as combined by cohort in the period between Day 1 and Day 225 post-administration in the MONARCH study. FIG. 36A shows a plot summarizing the reduction in the mean percent in seizure frequency from baseline in patients between ages 2-12 and between ages 13-18 years receiving 30 mg of ASO-1 as combined by cohort in the period between Day 1 and Day 225 post administration in the MONARCH study. FIG. 36B shows a plot summarizing the reduction in the mean percent in seizure frequency from baseline in patients between ages 2-12 and between ages 13-18 years receiving 45 mg of ASO-1 as combined by cohort in the period between Day 1 and Day 225 post administration in the MONARCH study.

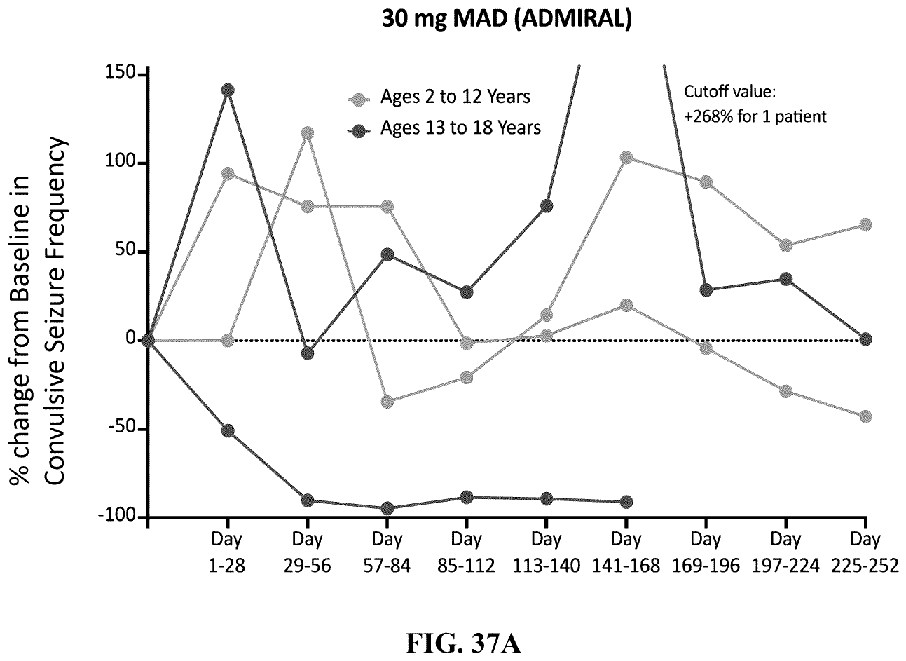

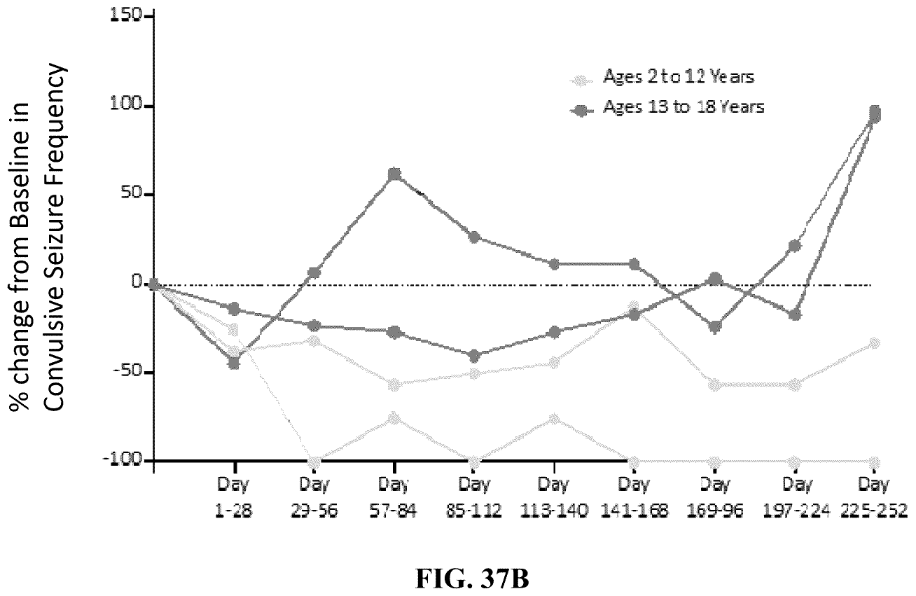

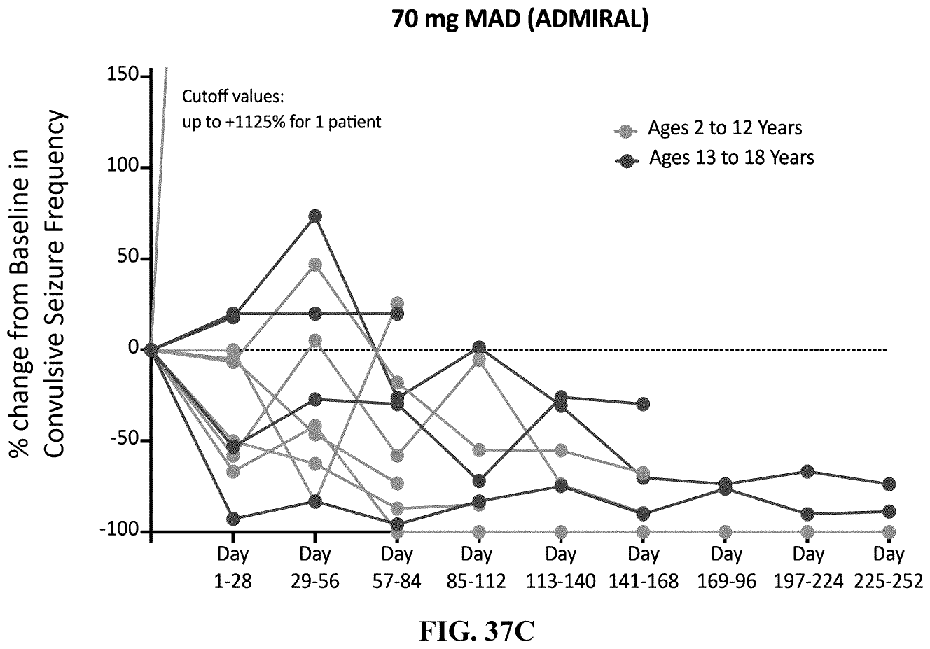

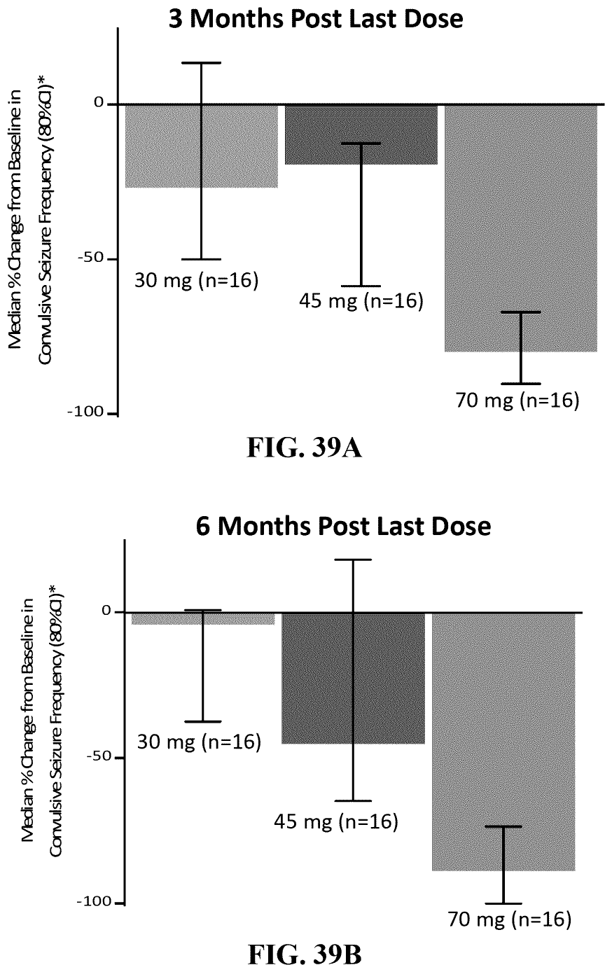

FIGS. 37A-37C show plots summarizing mean percent change in seizure frequency from baseline in patients between ages 2-12 and between ages 13-18 years receiving 30 mg, 45 mg, or 70 mg of ASO-1 as combined by cohort in the period between Day 1 and Day 252 post administration in the ADMIRAL study. FIG. 37A shows a plot summarizing the reduction in the mean percent in seizure frequency from baseline in patients between ages 2-12 and between ages 13-18 years receiving 30 mg of ASO-1 as combined by cohort in the period between Day 1 and Day 252 post administration in the ADMIRAL study. When measured at the end of 3 months post administration of the last dose, patients receiving 30 mg of ASO-1 showed an average reduction in Convulsive Seizure Frequency (CSF) of about 27% (FIG. 39A). When measured at the end of 6 months post administration of the last dose, patients receiving 30 mg of ASO-1 showed an average reduction in Convulsive Seizure Frequency (CSF) of about 5% (FIG. 39B).

FIG. 37B shows a plot summarizing the reduction in the mean percent in seizure frequency from baseline in patients between ages 2-12 and between ages 13-18 years receiving 45 mg of ASO-1 as combined by cohort in the period between Day 1 and Day 252 post administration in the ADMIRAL study. When measured at the end of 3 months post administration of the last dose, patients receiving 45 mg of ASO-1 showed an average reduction in Convulsive Seizure Frequency (CSF) of about 20% (FIG. 39A). When measured at the end of 6 months post administration of the last dose, patients receiving 45 mg of ASO-1 showed an average reduction in Convulsive Seizure Frequency (CSF) of about 45% (FIG. 39B).

FIG. 37C shows a plot summarizing the reduction in the mean percent in seizure frequency from baseline in patients between ages 2-12 and between ages 13-18 years receiving 70 mg of ASO-1 as combined by cohort in the period between Day 1 and Day 252 post administration in the ADMIRAL study. When measured at the end of 3 months post administration of the last dose, patients receiving 70 mg of ASO-1 showed an average reduction in Convulsive Seizure Frequency (CSF) of about 80% (FIG. 39A). When measured at the end of 6 months post administration of the last dose, patients receiving 70 mg of ASO-1 showed an average reduction in Convulsive Seizure Frequency (CSF) of about 90% (FIG. 39B).

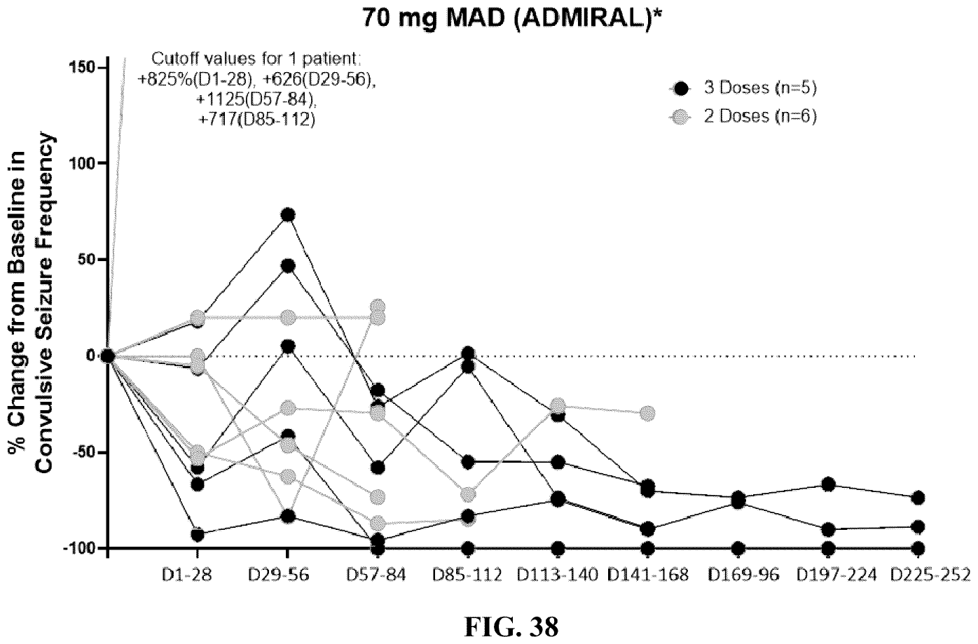

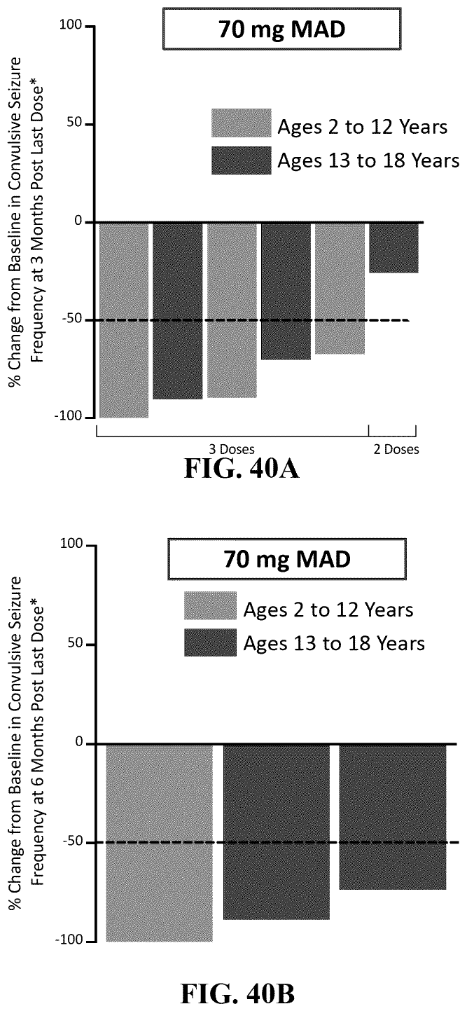

Patients (2-18 years) treated with 3 doses of 70 mg showed a differentiated response compared with patients treated with 2 doses of 70 mg of ASO-1 (FIG. 40A). FIG. 38 shows a plot summarizing mean percent change in seizure frequency from baseline in patients between ages 2-12 and between ages 13-18 years receiving 2 or 3 doses of 70 mg of ASO-1, as combined by cohort in the period between Day 1 and Day 252 post administration in the ADMIRAL study. Patients treated with 3 doses of 70 mg ASO-1 showed about 70% reduction in seizure frequency in patients between the age of 13-18 when measured at the end of the 3-month period following the administration of the last dose (FIG. 40A). In the case of patients between the age of 2-12 years treated with 3 doses of 70 mg ASO-1, a reduction in seizure frequency of greater than 85% was observed (FIG. 40A) when measured at the end of the 3-month period following the administration of the last dose.

Patients treated with 3 doses of 70 mg showed an average reduction in convulsive seizure frequency by about 80% by the D225- or D252 mark (FIG. 39). When measured at the end of 6 months post administration of the last dose, patients between the ages of 13-18 receiving 2 or 3 70 mg doses of ASO-1 showed an average reduction in Convulsive Seizure Frequency (CSF) of about 75% (FIG. 40B). When measured at the end of 6 months post administration of the last dose, patients receiving 70 mg of ASO-1 showed an average reduction in Convulsive Seizure Frequency (CSF) of about 98% (FIG. 40B) in children between the ages of 2-12 (FIG. 40B).

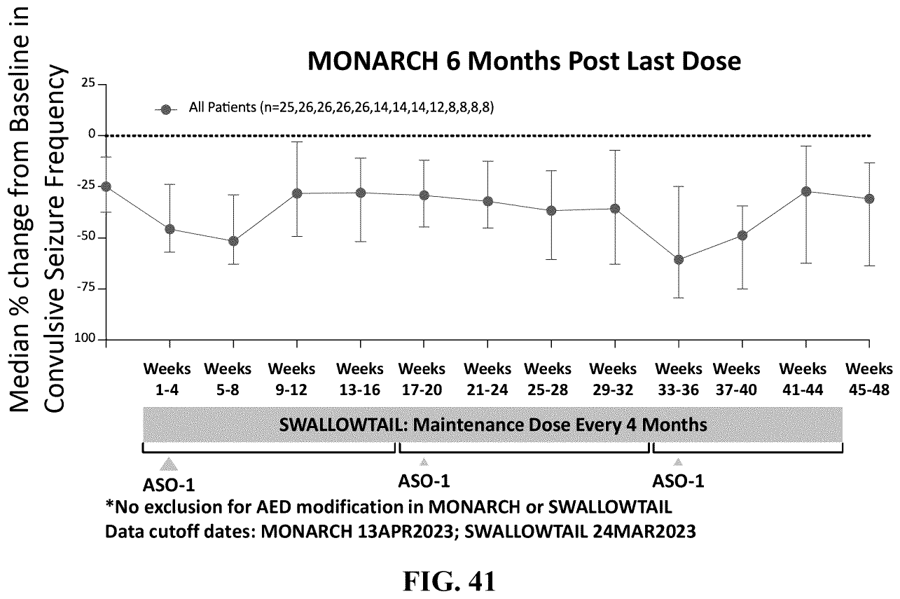

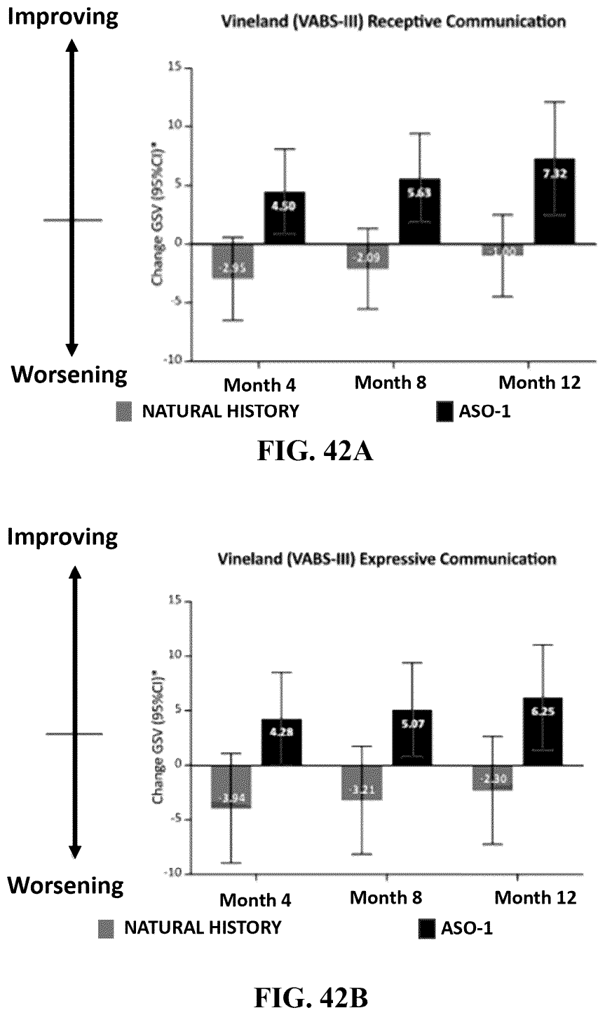

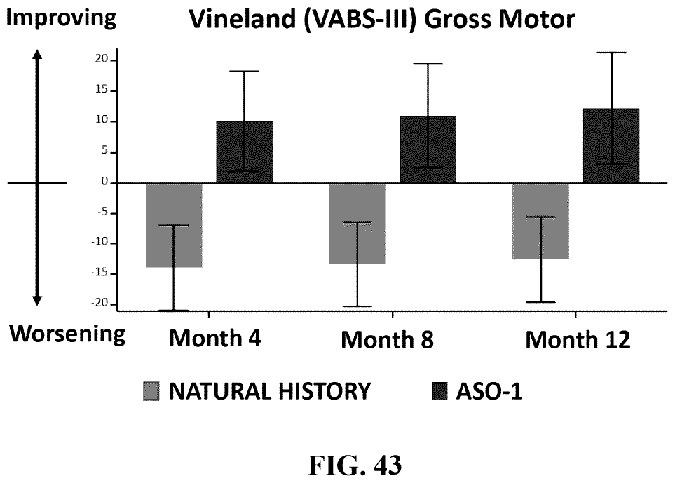

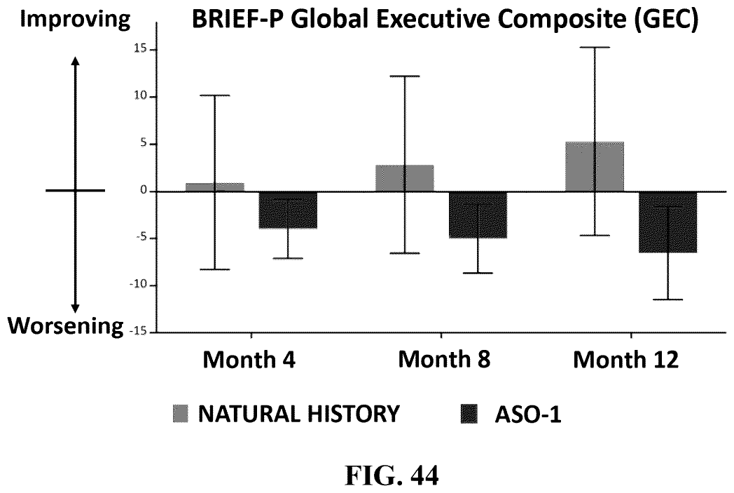

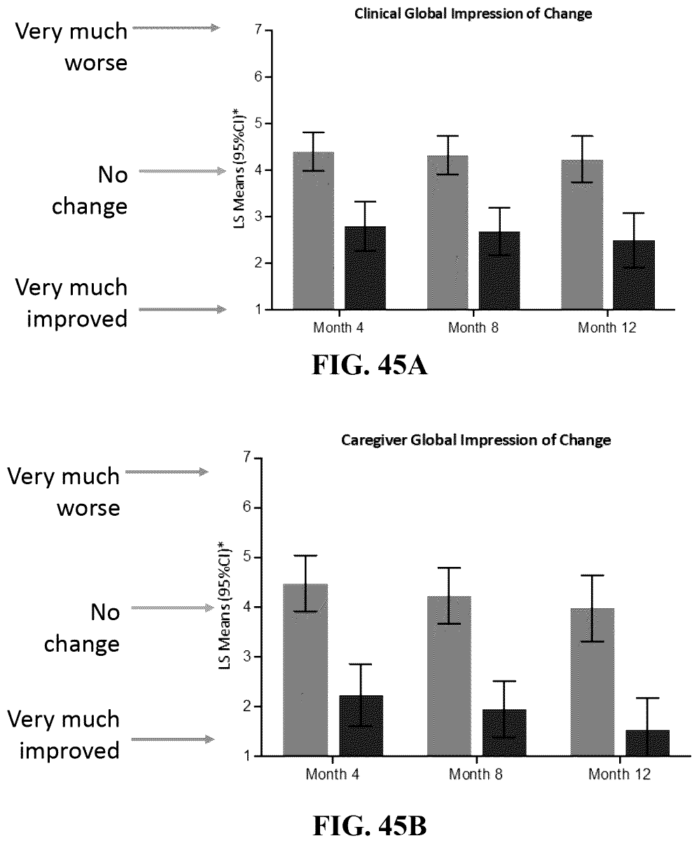

In the Open-label Extension study, sustained reductions in CSF were observed with ASO-1 doses of 30 mg or 45 mg every four months (FIG. 41). Patients receiving ASO-1 for over one year of dosing showed an improvement in receptive communication as compared to observed natural history of the disease measured on the Vineland (VABS-III) scale (FIG. 42A). Patients receiving ASO-1 for over one year of dosing showed an improvement in expressive communication as compared to observed natural history of the disease measured on the Vineland (VABS-III) scale (FIG. 42B). Patients receiving ASO-1 for over one year of dosing showed an improvement in gross motor skills as compared to observed natural history of the disease measured on the Vineland (VABS-III) scale (FIG. 43). Patients receiving ASO-1 for over one year of dosing showed an improvement in executive function as compared to observed natural history of the disease measured on the Vineland (VABS-III) scale (FIG. 44). Patients receiving ASO-1 for over one year of dosing showed a substantial improvement in overall condition. For example, patients receiving ASO-1 for over one year of dosing showed a substantial improvement in overall condition when measured under the Clinician Global Impression of change (FIG. 45A). Further, patients receiving ASO-1 for over one year of dosing showed a substantial improvement in overall condition when measured under the Caregiver Global Impression of change (FIG. 45B).

For the behavioral measurements in FIGS. 42-45, mixed model repeated measures were performed with AR(1) covariance structure. Baseline covariates in BUTTERFLY were matched to SWALLOWTAIL. Analysis included all patients who received 30 or 45 mg for all doses in SWALLOWTAIL. For CGI-C, BUTTERFLY sample size: n=32 at Month 3, n=29 at Month 12; and for CaGI-C, BUTTERFLY sample size: n=27 at Month 3, n=24 at Month 12. For both CGI-C and CaGI-C, SWALLOWTAIL sample size: n=25 at Week 16, n=9 at Week 48 and n=5 at Week 64. CGI and CaGI in BUTTERFLY were adapted for cognition. CGI-C-Clinical Global Impression of Change and CaGI-C-Caregiver Global Impression of Change.

TABLE 11
Demographics for patients treated with >=1 dose of ASO-1

| | Total, n (%) |
|---|---|
| N | 74 |

Age at Screening, y

| | |
|---|---|
| Mean (SD) | 10.2 years (5.05) |
| Median (min, max) | 10.5 years (2, 18) |

Sex

| | |
|---|---|
| Female | 39 (52.7%) |
| Male | 35 (47.3%) |

Number of Concomitant Anti-Seizure Medications

| | |
|---|---|
| ≥3 | 60 (81.1%) |
| >4 | 37 (50.0%) |

Concomitant Fenfluramine

| | |
|---|---|
| % | 36 (48.6%) |

Baseline Convulsive Seizure Frequency per 28 days

| | |
|---|---|
| Median (min, max) | 17.5 (1, 2335) |

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-173. (canceled)

174. A method of treating or reducing the likelihood of developing a disease or condition characterized by a reduced expression or function of Na_{V}1.1 protein in a human subject in need thereof, the method comprising administering to the human subject a pharmaceutical composition comprising a compound according to the following chemical structure: 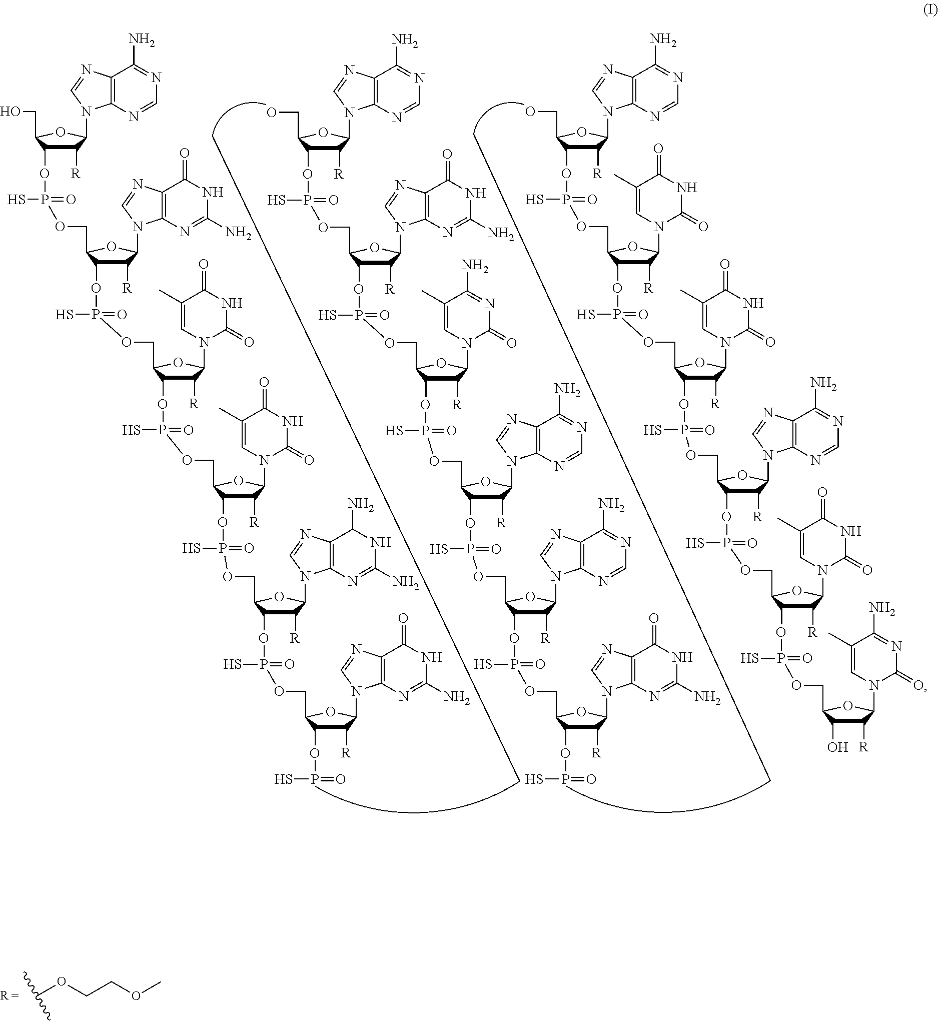
or a salt thereof, at a first dose, one or more loading doses, and one or more maintenance doses, thereby treating or reducing the likelihood of developing the disease or condition in the human subject;
wherein the administration of the first dose is followed by administration of one or more loading doses,
wherein the one or more maintenance doses is administered after the administration of the one or more loading doses,
wherein the first dose contains the compound at a first dose amount of from about 25 to about 80 mg,
wherein:
(a) a subsequent dose of the one or more maintenance doses is higher than a previously administered maintenance dose,
(b) a subsequent dose of the one or more maintenance doses is the same as a previously administered maintenance dose, or
(c) a subsequent dose of the one or more maintenance doses is lower than a previously administered maintenance dose; and
wherein the disease or condition is Dravet Syndrome.

175. The method of claim 174, wherein the compound has the following structure:

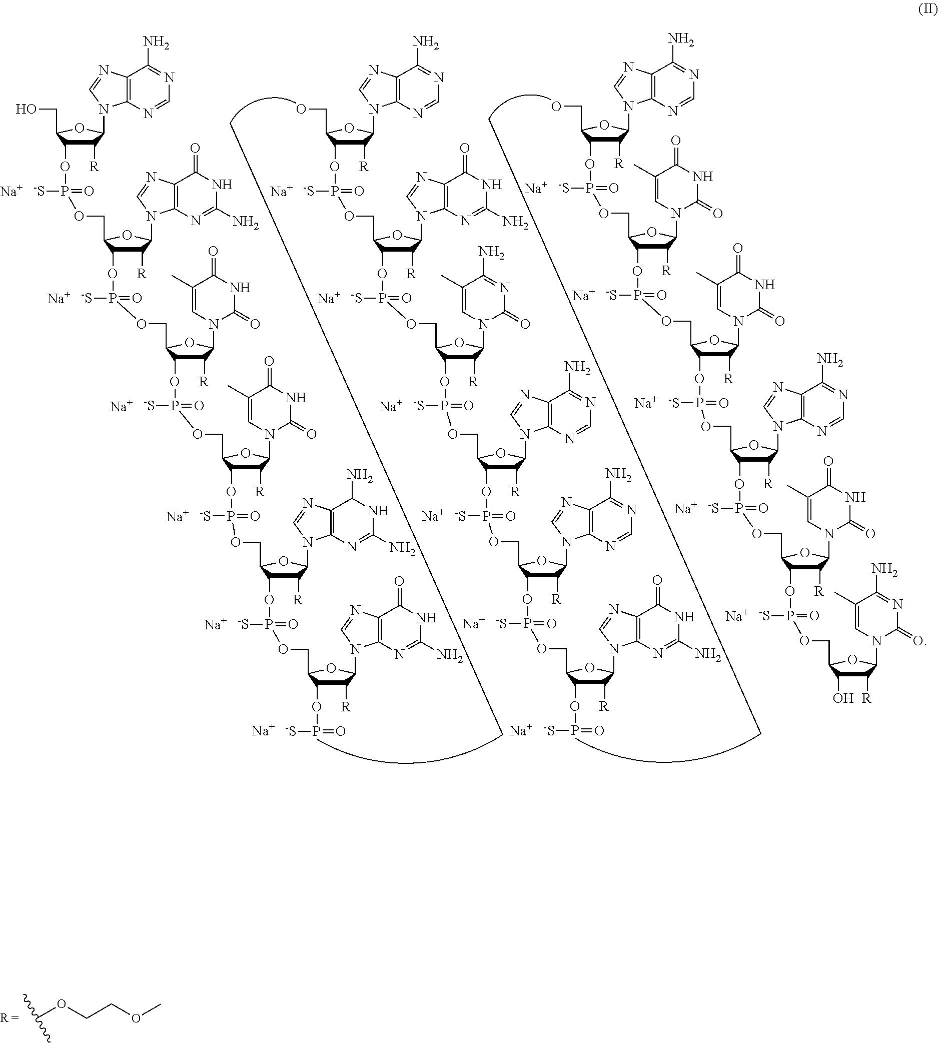

176. The method of claim 174, wherein the method comprises administering to the human subject the pharmaceutical composition comprising the compound at the first dose of about 30, 45, or 70 mg.

177. The method of claim 176, wherein the first dose amount is about 30 mg.

178. The method of claim 176, wherein the first dose amount is about 45 mg.

179. The method of claim 176, wherein the first dose amount is about 70 mg.

180. The method of claim 174, wherein each of the one or more loading doses contains the compound at a dose amount that is the same as the first dose amount.

181. The method of claim 174, wherein the method comprises administering the first dose, followed by a single loading dose and the one or more maintenance doses.

182. The method of claim 174, wherein the dose amount of the one or more maintenance doses is about 45 mg.

183. The method of claim 174, wherein the dose amount of the one or more maintenance doses is about 70 mg.

184. The method of claim 174, wherein the dose amount of the one or more maintenance doses is at least about 30% lower than the first dose amount.

185. The method of claim 174, wherein the subsequent dose of the one or more maintenance doses is the same as the previously administered maintenance dose.

186. The method of claim 174, wherein dose frequency of the administration of the pharmaceutical composition is maintained or reduced following an indication that the previous dose is effective.

187. The method of claim 174, wherein dose frequency of the administration of the pharmaceutical composition is reduced following an indication that the previous dose is not tolerated.

188. The method of claim 174, wherein the first dose amount is effective to reduce seizure frequency and/or improve cognition and/or behavior for at least about one year, compared to a human subject not treated with the compound.

189. The method of claim 174, wherein the dose amount of the one or more maintenance doses is effective to reduce seizure frequency and/or improve cognition and/or behavior for at least about one year, compared to observed natural history of the disease.

190. The method of claim 174, wherein the pharmaceutical composition is a liquid composition.

191. The method of claim 174, wherein the compound is dissolved or suspended in a diluent and the first dose has a volume of 5 ml or higher.

192. The method of claim 191, wherein the diluent comprises a cerebral spinal fluid (CSF) sample from the human subject or an artificial cerebral spinal fluid (aCSF) solution.

193. The method of claim 174, wherein the brain concentration of the compound after administration of the pharmaceutical composition is at least 1, 2, 4, 6, 8, or 10 μg/ml.
